(19) United States
(12) Patent Application Publication
(10) Pub. No.: US 2017/0002060 A1
(43) Pub. Date: Jan. 5, 2017

(54) POLYNUCLEOTIDES FOR THE IN VIVO PRODUCTION OF ANTIBODIES

(71) Applicant: MODERNA THERAPEUTICS, INC., Cambridge, MA (US)

(72) Inventors: Joseph Beene Bolen, Cambridge, MA (US); Axel Bouchon, Cambridge, MA (US); Giuseppe Ciaramella, Sudbury, MA (US); Eric Yi-Chun Huang, Boston, MA (US)

(21) Appl. No.: 15/106,000

(22) PCT Filed: Jan. 8, 2015
(86) PCT No.: PCT/US15/10547

§ 371 Date: Jun. 17, 2016

Related U.S. Application Data
(60) Provisional application No. 61/924,767, filed on Jan. 8, 2014.
(60) Provisional application No. 61/924,770, filed on Jan. 8, 2014.
(60) Provisional application No. 61/924,773, filed on Jan. 8, 2014.
(60) Provisional application No. 61/924,774, filed on Jan. 8, 2014.
(60) Provisional application No. 61/924,776, filed on Jan. 8, 2014.
(60) Provisional application No. 61/924,779, filed on Jan. 8, 2014.
(60) Provisional application No. 61/924,781, filed on Jan. 8, 2014.
(60) Provisional application No. 61/927,783, filed on Jan. 15, 2014.
(60) Provisional application No. 61/924,787, filed on Jan. 8, 2014.
(60) Provisional application No. 61/924,795, filed on Jan. 8, 2014.
(60) Provisional application No. 61/924,799, filed on Jan. 8, 2014.
(60) Provisional application No. 61/924,802, filed on Jan. 8, 2014.
(60) Provisional application No. 61/924,805, filed on Jan. 8, 2014.
(60) Provisional application No. 61/924,807, filed on Jan. 8, 2014.
(60) Provisional application No. 61/924,810, filed on Jan. 8, 2014.
(60) Provisional application No. 61/924,811, filed on Jan. 8, 2014.
(60) Provisional application No. 61/924,817, filed on Jan. 8, 2014.
(60) Provisional application No. 61/924,819, filed on Jan. 8, 2014.
(60) Provisional application No. 61/924,820, filed on Jan. 8, 2014.
(60) Provisional application No. 61/924,754, filed on Jan. 8, 2014.
(60) Provisional application No. 61/924,763, filed on Jan. 8, 2014.
(60) Provisional application No. 61/924,768, filed on Jan. 8, 2014.
(60) Provisional application No. 61/924,775, filed on Jan. 8, 2014.
(60) Provisional application No. 61/924,791, filed on Jan. 8, 2014.
(60) Provisional application No. 61/924,804, filed on Jan. 8, 2014.
(60) Provisional application No. 61/924,812, filed on Jan. 8, 2014.
(60) Provisional application No. 61/924,827, filed on Jan. 8, 2014.
(60) Provisional application No. 61/924,834, filed on Jan. 8, 2014.
(60) Provisional application No. 61/924,841, filed on Jan. 8, 2014.
(60) Provisional application No. 61/924,846, filed on Jan. 8, 2014.
(60) Provisional application No. 61/924,850, filed on Jan. 8, 2014.
(60) Provisional application No. 61/924,715, filed on Jan. 8, 2014.

(51) Int. Cl. C07K 16/00

(52) U.S. Cl. CPC C07K 16/00; C07K 2317/569; C07K 2317/622; C07K 2317/31

(57) ABSTRACT

The invention relates to compositions and methods for the preparation, manufacture and therapeutic use of polynucleotides encoding an antibody, variant or fragment.

### CROSS REFERENCE TO RELATED APPLICATIONS

This application claims priority to U.S. Provisional Patent Application No. 61/924,767 filed Jan. 8, 2014, entitled Polynucleotides For The In Vivo Production of Antibodies, U.S. Provisional Patent Application No. 61/924,770 filed Jan. 8, 2014, entitled Polynucleotides For The In Vivo Production of Antibodies, U.S. Provisional Patent Application No. 61/924,773 filed Jan. 8, 2014, entitled Polynucleotides For The In Vivo Production of Antibodies, U.S. Provisional Patent Application No. 61/924,774 filed Jan. 8, 2014, entitled Polynucleotides For The In Vivo Production of Antibodies, U.S. Provisional Patent Application No. 61/924,776 filed Jan. 8, 2014, entitled Polynucleotides For The In Vivo Production of Antibodies, U.S. Provisional Patent Application No. 61/924,779 filed Jan. 8, 2014, entitled Polynucleotides For The In Vivo Production of Antibodies, U.S. Provisional Patent Application No. 61/924,781 filed Jan. 8, 2014, entitled Polynucleotides For The In Vivo Production of Antibodies, U.S. Provisional Patent Application No. 61/924,783 filed Jan. 8, 2014, entitled Polynucleotides For The In Vivo Production of Antibodies, U.S. Provisional Patent Application No. 61/924,787 filed Jan. 8, 2014, entitled Polynucleotides For The In Vivo Production of Antibodies, U.S. Provisional Patent Application No. 61/924,795 filed Jan. 8, 2014, entitled Polynucleotides For The In Vivo Production of Antibodies, U.S. Provisional Patent Application No. 61/924,799 filed Jan. 8, 2014, entitled Polynucleotides For The In Vivo Production of Antibodies, U.S. Provisional Patent Application No. 61/924,802 filed Jan. 8, 2014, entitled Polynucleotides For The In Vivo Production of Antibodies, U.S. Provisional Patent Application No. 61/924,805 filed Jan. 8, 2014, entitled Polynucleotides For The In Vivo Production of Antibodies, U.S. Provisional Patent Application No. 61/924,807 filed Jan. 8, 2014, entitled Polynucleotides For The In Vivo Production of Antibodies, U.S. Provisional Patent Application No. 61/924,810 filed Jan. 8, 2014, entitled Polynucleotides For The In Vivo Production of Antibodies, U.S. Provisional Patent Application No. 61/924,811 filed Jan. 8, 2014, entitled Polynucleotides For The In Vivo Production of Antibodies, U.S. Provisional Patent Application No. 61/924,817 filed Jan. 8, 2014, entitled Polynucleotides For The In Vivo Production of Antibodies, U.S. Provisional Patent Application No. 61/924,819 filed Jan. 8, 2014, entitled Polynucleotides For The In Vivo Production of Antibodies, U.S. Provisional Patent Application No. 61/924,820 filed Jan. 8, 2014, entitled Polynucleotides For The In Vivo Production of Antibodies, U.S. Provisional Patent Application No. 61/924,754 filed Jan. 8, 2014, entitled Polynucleotides For The In Vivo Production of Antibodies, U.S. Provisional Patent Application No. 61/924,763 filed Jan. 8, 2014, entitled Polynucleotides For The In Vivo Production of Antibodies, U.S. Provisional Patent Application No. 61/924,768 filed Jan. 8, 2014, entitled Polynucleotides For The In Vivo Production of Antibodies, U.S. Provisional Patent Application No. 61/924,775 filed Jan. 8, 2014, entitled Polynucleotides For The In Vivo Production of Antibodies, U.S. Provisional Patent Application No. 61/924,791 filed Jan. 8, 2014, entitled Polynucleotides For The In Vivo Production of Antibodies, U.S. Provisional Patent Application No. 61/924,804 filed Jan. 8, 2014, entitled Polynucleotides For The In Vivo Production of Antibodies, U.S. Provisional Patent Application No. 61/924,812 filed Jan. 8, 2014, entitled Polynucleotides For The In Vivo Production of Antibodies, U.S. Provisional Patent Application No. 61/924,827 filed Jan. 8, 2014, entitled Polynucleotides For The In Vivo Production of Antibodies, U.S. Provisional Patent Application No. 61/924,834 filed Jan. 8, 2014, entitled Polynucleotides For The In Vivo Production of Antibodies, U.S. Provisional Patent Application No. 61/924,841 filed Jan. 8, 2014, entitled Polynucleotides For The In Vivo Production of Antibodies, U.S. Provisional Patent Application No. 61/924,846 filed Jan. 8, 2014, entitled Polynucleotides For The In Vivo Production of Antibodies and U.S. Provisional Patent Application No. 61/924,850 filed Jan. 8, 2014, entitled Polynucleotides For The In Vivo Production of Antibodies, U.S. Provisional Patent Application No. 61/993,715 filed May 15, 2014, entitled Polynucleotides For The In Vivo Production of Antibodies, the contents of each of which are herein incorporated by reference in its entirety.

### REFERENCE TO SEQUENCE LISTING

The present application is being filed along with a Sequence Listing in electronic format. The Sequence Listing is provided as a file entitled 2030.1080PCT-M080.20_SL.txt, created on Jan. 7, 2015 which is 567,081 bytes in size. The information in the electronic format of the sequence listing is incorporated herein by reference in its entirety.

### FIELD OF THE INVENTION

The invention relates to compositions, methods, processes, kits and devices for the design, preparation, manufacture and/or formulation of antibodies in vivo.

### BACKGROUND OF THE INVENTION

Antibodies, also known as immunoglobulins, are glycoproteins produced by B cells. Using a unique and highly evolved system of recognition, antibodies can recognize a target and tag a target epitope, foreign entity or invading microbe for attack by the immune system thereby neutralizing its effect. The production of antibodies is the main function of the humoral immune system. Antibodies are secreted by a plasma cell which is a type of white blood cell.

Antibodies occur in two physical forms, a soluble form that is secreted from the cell, and a membrane-bound form that is attached to the surface of a B cell and is referred to as the B cell receptor (BCR). Soluble antibodies are released into the blood and tissue fluids, as well as many secretions to continue to survey for invading microorganisms.

The majority of antibodies comprise two heavy chains and two light chains. There are several different types of antibody heavy chains, and several different kinds of antibodies, which are grouped into different isotypes based on which heavy chain they possess. Five different antibody isotypes isotypes (IgA, IgD, IgE, IgG and IgM) are known in mammals and trigger a different immune response for each different type of foreign object, epitope or microbe they encounter.

Frequently the binding of an antibody to an antigen has no direct biological effect. Rather, the significant biological effects are a consequence of secondary “effector functions” of antibodies. The immunoglobulins mediate a variety of these effector functions. These functions include fixation of complement, binding of phagocytic cells, lymphocytes, platelets, mast cells, and basophils which have immunoglobulin receptors. This binding can activate the cells to perform some function. Some antibodies or immunoglobulins also bind to receptors on placental trophoblasts, which results in transfer of the immunoglobulin across the placenta. As a result, the transferred maternal antibodies provide immunity to the fetus and newborn.

Development of monoclonal antibodies as therapeutics, while on the rise, still suffers from high costs and predominantly a one-to-one targeting approach. Many antibodies are undergoing clinical testing with the majority directed to the treatment of oncology and immunologic disorders.

Currently, the majority of antibodies are generated using recombinant or cloning strategies and product heterogeneity is common to monoclonal antibody and other recombinant biological production. Such heterogeneity is typically introduced either upstream during expression or downstream during manufacturing. Recombinant antibody engineering involves the use of viruses or yeast to create antibodies, rather than mice which are used in cloning strategies. All of these however, suffer from drawbacks associated with the systems used for generation including degree of purity, speed of development, cross reactivity, low affinity and variable specificity.

Production of antibodies in vivo whether via direct translation of an encoding polynucleotide that elicits antibody production by the body can address most, if not all, of the problems associated with traditional antibody technology.

The present invention provides such methods and compositions for the in vivo production of antibodies.

### SUMMARY OF THE INVENTION

Described herein are compositions, methods, processes, kits and devices for the design, preparation, and/or manufacture of antibodies in vivo.

The details of various embodiments of the invention are set forth in the description below. Other features, objects, and advantages of the invention will be apparent from the description and the drawings, and from the claims.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 is a PRIOR ART schematic of the five classes of known antibodies.

The figure was taken from the URL www2.estrellamountain.edu/faculty/farabee/biobk/biobookimmun.html.

FIG. 2 is a schematic comparing (A) the human IgG antibody structure to (B) a bicistronic antibody of the invention.

FIG. 3 is a schematic of two types of antibodies of the present invention. (A) an scFv antibody and (B) a single domain IgG antibody.

FIG. 4 is a schematic of two types of antibodies of the present invention. (A) a bispecific antibody and (B) a single domain IgA antibody.

FIG. 5 is a schematic of certain polynucleotide constructs of the present invention illustrating (A) the modular design of the encoding polynucleotides and (B) the domains or regions of a standard antibody unit.

### DETAILED DESCRIPTION

It is of great interest in the fields of therapeutics, diagnostics, reagents and for biological assays to be able design, synthesize and deliver a nucleic acid, e.g., a ribonucleic acid (RNA) inside a cell, whether in vitro, in vivo, in situ or ex vivo, such as to effect physiologic outcomes which are beneficial to the cell, tissue or organ and ultimately to an organism. One beneficial outcome is to cause intracellular translation of the nucleic acid and production of at least one encoded peptide or polypeptide of interest.

Described herein are compositions (including pharmaceutical compositions) and methods for the design, preparation, manufacture and/or formulation of antibodies where at least one component of the antibody is encoded by a polynucleotide. As such the present invention is directed, in part, to polynucleotides, specifically IVT polynucleotides, chimeric polynucleotides and/or circular polynucleotides encoding one or more antibodies and/or components thereof.

Also provided are systems, processes, devices and kits for the selection, design and/or utilization of the antibodies described herein.

According to the present invention, the polynucleotides are preferably modified in a manner as to avoid the deficiencies of or provide improvements over other antibody molecules of the art.

Provided herein, therefore, are antibodies or portions thereof encoded by polynucleotide(s) and antibody compositions comprising at least one polynucleotide which have been designed to produce a therapeutic outcome and optionally improve one or more of the stability and/or clearance in tissues, receptor uptake and/or kinetics, cellular access, engagement with translational machinery, mRNA half-life, translation efficiency, protein production capacity, secretion efficiency (when applicable), accessibility to circulation, protein half-life and/or modulation of a cell's status, antibody target affinity and/or specificity, reduction of antibody cross reactivity, increase of antibody purity, increase or alteration of antibody effector function and/or antibody activity.

The methods of the present invention are and can be utilized to engineer novel polynucleotides for the in vivo production of antibodies in such a manner as to provide improvements over standard antibody technology.

In some embodiments, the polynucleotides are designed to produce one or more antibodies, or combinations of antibodies selected from the group consisting of IgA, IgG, IgM, IgE, and IgD.

The resultant antibodies expressed in a cell, tissue or organism from the polynucleotides of the present invention may have the following properties which mirror those of the natural isotype. They may also exhibit improved properties over the native or natural isotype.

TABLE 1
Features of antibody isotypes

| | | | Complement | |
|---|---|---|---|---|
| Isotype | Presence | Function | Fixation | Structure |
| IgG | Appears as | Binding to | Fixation of | Exists as monomer of two |
| | major Ig | macrophages, | complement | heavy and two light chains |
| | in serum; | monocytes; | | |
| | may | may act as | | |
| | appear | opsonin; Cross | | |
| | greater | placental | | |
| | than 75% | transport | | |
| IgA | Second | Binds PMNs | Does not fix | Serum form is monomer; |
| | most | and some | complement | secreted form is dimer with |
| | common | lymphocytes;; | unless aggregated | J chain; secretory piece |
| | Ig in | major class in | | made in epithelial cells |
| | serum | secretions such | | |
| | | as tears, saliva, | | |
| | | colostrum, | | |
| | | mucous | | |
| IgM | Third most | Made by fetus | Fixation of | Exist as pentamer or |
| | common | and virgin B | complement; | monomer; all heavy chains |
| | Ig in | cells; B cell | destruction of | identical; all light chains |
| | serum | surface Ig | microorganisms | identical; valence of 10Extra |
| | | lacks J chain | by agglutination | domain on CH4 chain; |
| | | | and/or clumping | additional protein bound via |
| | | | | disulfide known as J chain |
| | | | | for polymerization; cells via |
| | | | | Fc receptors |
| IgE | Least | Involved in | Does not fix | Exists as monomer with |
| | abundant | allergic | complement | extra domain in constant |
| | in serum | reactions; | | region; binds to Fc receptors |
| | | release of | | on basophils and mast cells |
| | | mediators of | | before antigen interaction |
| | | allergic | | |
| | | symptoms; | | |
| | | role in | | |
| | | parasitic | | |
| | | helminth | | |
| | | disease | | |
| IgD | Low levels | Found on B | Does not bind | Only exists as monomer; |
| | in serum | cell surface as | complement | additional amino acids at C- |
| | | receptor for | | terminus for membrane |
| | | antigens; | | anchoring; associates with |
| | | | | Ig-alpha and Ig-beta |

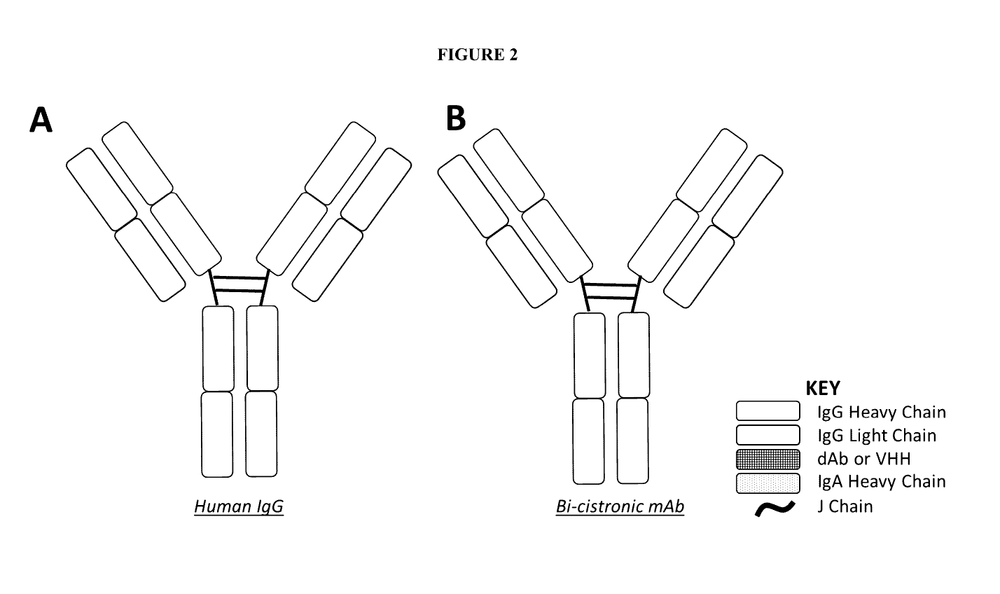

In one embodiment, the polynucleotides described herein may encode a human IgG construct as described in FIG. 2A.

### I. COMPOSITIONS OF THE INVENTION

### Polynucleotides as Components of Antibody Compositions

The compositions of the present invention comprise polynucleotides which encode the antibody, fragments of the antibody or variants of the antibody and are collectively referred to as “polynucleotides” “antibody polynucleotides” “constructs” or “antibody constructs.” Compositions of the invention which comprise at least one polynucleotide are referred to as “compositions” or “antibody compositions.” The polypeptides encoded by the polynucleotides are collectively referred to as polypeptides, whether the polypeptides are variants, fragments or the entire antibody.

The polynucleotides or compositions may be administered as a targeted adaptive vaccine, as disclosed in copending International application number PCT/US2014/69155, filed Dec. 8, 2014 (Attorney Docket Number M073), the contents of which are incorporated herein by reference in their entirety.

In one embodiment, the polynucleotides or compositions be administered as a neutralizing antibody.

Where necessary, a tolerizing polynucleotide may be included, such as those described in copending International Application No. PCT/US2014/061104, filed Oct. 17, 2014 (Attorney Docket No. M059), the contents of which are incorporated herein by reference in their entirety.

### Polynucleotides

The present invention provides nucleic acid molecules, specifically polynucleotides which, in some embodiments, encode one or more peptides or polypeptides of interest. Such peptides or polypeptides, according to the invention are those derived from at least one of the antibodies described herein. The term “nucleic acid,” in its broadest sense, includes any compound and/or substance that comprise a polymer of nucleotides. These polymers are often referred to as polynucleotides.

Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or hybrids or combinations thereof.

In one embodiment, linear polynucleotides encoding one or more antibody constructs of the present invention which are made using only in vitro transcription (IVT) enzymatic synthesis methods are referred to as “IVT polynucleotides.” Methods of making IVT polynucleotides are known in the art and are described in co-pending International Publication No. WO2013151666 filed Mar. 9, 2013 (Attorney Docket Number M300), the contents of which are incorporated herein by reference in their entirety.

In another embodiment, the polynucleotides of the present invention which have portions or regions which differ in size and/or chemical modification pattern, chemical modification position, chemical modification percent or chemical modification population and combinations of the foregoing are known as “chimeric polynucleotides.” A “chimera” according to the present invention is an entity having two or more incongruous or heterogeneous parts or regions. As used herein a “part” or “region” of a polynucleotide is defined as any portion of the polynucleotide which is less than the entire length of the polynucleotide. Such constructs are taught in for example copending International Patent Application No. PCT/US2014/053907, filed Sep. 3, 2014 (Attorney Docket Number M57), the contents of which are incorporated herein by reference in their entirety.

In yet another embodiment, the polynucleotides of the present invention that are circular are known as “circular polynucleotides” or “circP.” As used herein, “circular polynucleotides” or “circP” means a single stranded circular polynucleotide which acts substantially like, and has the properties of, an RNA. The term “circular” is also meant to encompass any secondary or tertiary configuration of the circP. Such constructs are taught in for example copending International Application No. PCT/US2014/053904, filed Sep. 3, 2014 (Attorney Docket Number M051) and International Application No. PCT/US2014/053907, filed Sep. 3, 2014 (Attorney Docket Number M057), the contents of each of which are incorporated herein by reference in their entirety.

In some embodiments, the polynucleotide includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In one embodiment, the polynucleotides of the present invention may encode at least one peptide or polypeptide of interest.

According to the present invention, the polypeptide of interest comprises at least one antibody described herein, or fragments or variants thereof.

In one embodiment, the polynucleotides have a modular design to encode the polypeptides of interest.

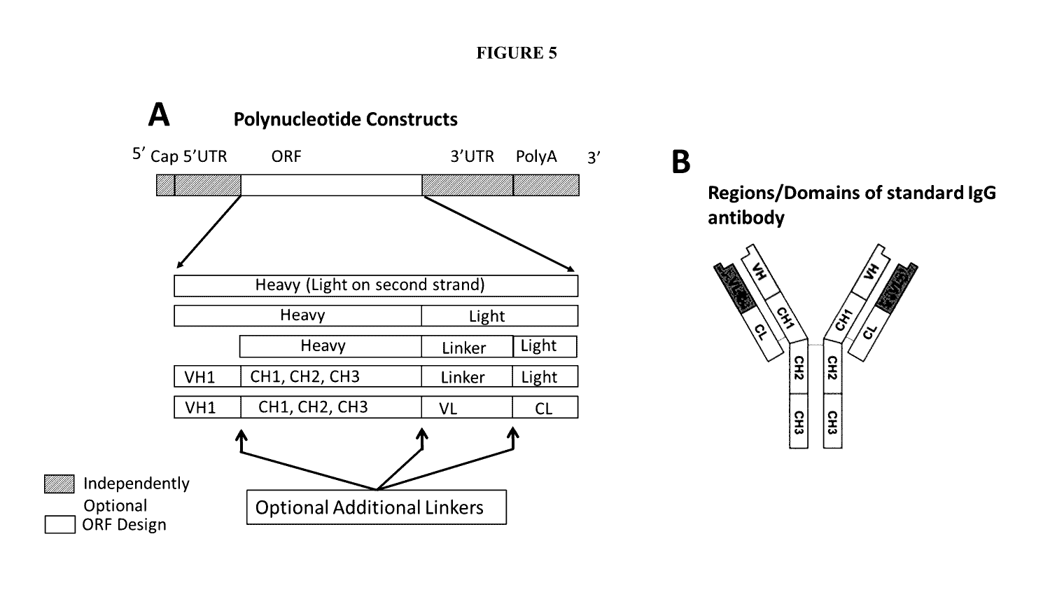

In one embodiment, the polynucleotides have a modular design to encode at least one of the antibodies, fragments or variants thereof described herein. As a non-limiting example, as shown in FIG. 5A, the polynucleotide construct may encode any of the following designs: (1) the heavy chain of an antibody, (2) the light chain of an antibody, (3) the heavy and light chain of the antibody, (4) the heavy chain and light chain separated by a linker, (5) the VH1, CH1, CH2, CH3 domains, a linker and the light chain and (6) the VH1, CH1, CH2, CH3 domains, VL region, and the light chain. Any of these designs may also comprise optional linkers between any domain and/or region.

In one embodiment, the polynucleotides have a modular design and ecode a polypeptide of interest such as, but not limited to, an antibody, fragment or variant thereof described herein. Shown in FIG. 5B are the domains or regions of a standard antibody unit.

### Abciximab Parent Molecule or Antibody

According to the present invention, abciximab polynucleotides or constructs and their associated abciximab compositions are designed to produce the abciximab antibody, a variant or a portion thereof in vivo.

Abciximab, also be referred to herein as REOPRO® was originally derived from 7E3 (original monoclonal), c7E3 Fab (chimeric human-mouse Fab or F(ab′)2 fragment and is provided under the brand name REOPRO®.

Abciximab is an anticoagulant agent. It comprises an Fab fragment derived from a chimeric monoclonal IgG antibody (from human and murine origins.) Abciximab was designed based on the development of a monoclonal antibody by Barry Coller referred to as 7E3 (Coller, B. S. 1985. J Clin Invest. 76(1):101-8.) Further studies by Coller et al demonstrated that F(ab′)2 fragments of 7E3 were able to inhibit platelet aggregation in a dose-dependent manner in dogs (Coller et al, 1985. Blood. 66(6):1456-9) and subsequent studies demonstrated a similar effect in humans (Ellis et al., 1993. Coron Artery Dis. 4(2):167-75.) Further developments of 7E3 led to the development of a chimeric antibody, c7E3 (Centocor, Malvern, Pa.), comprising a human-mouse genetic version. This version was also shown to have therapeutic benefits in humans, including a reduction in the number of patients experiencing myocardial infarction (MCI) after angioplasty (N Engl J Med 1994; 330:956-61.)

The antibody binds the human platelet glycoprotein receptor IIb/IIIa (GPIIb/IIIa.) GPIIb/IIIa is an integrin adhesion receptor family member and represents the primary surface receptor responsible for platelet aggregation. Binding of Abciximab to this receptor inhibits platelet aggregation occurring through adhesive molecule binding, such as fibrinogen and von Willebrand factor, for example. Abciximab has also been shown to bind vitronectin (also known as avβ3 integrin) receptor that is expressed by platelets as well as vascular endothelial and smooth muscle cells.

In some embodiments, polynucleotides of the present invention may encode one or more portions of the antibody produced by hybridoma HB8832 as described by U.S. Pat. Nos. 5,275,812 and 5,770,198, the contents of each of which are herein incorporated by reference in their entirety. Such polynucleotide-derived antibodies may comprise one or more portions of the antigen binding region of antibodies produced by hybridoma HB8832. In some cases, antibodies encoded by polynucleotides of the present invention may comprise fragments of the antibody produced by hybridoma HB8832. Such fragments may include, but are not limited to Fab fragments or F(ab′)2 fragments, such as those described in U.S. Pat. No. 5,440,020, the contents of which are herein incorporated by reference their entirety.

Abciximab polypeptides or antibodies encoded by polynucleotides of the present invention may associate with the GPIIb/IIIa receptor. Such associations may prevent GPIIb/IIIa binding through steric hindrance as opposed to direct association with the amino acids of the receptor thought to form bonds with the GPIIb/IIIa RGD (arginine-glycine-aspartic acid) domain. Polynucleotide-derived antibodies disclosed herein may also bind vitronectin. Such binding may prevent activity associated with this receptor, including, but not limited to cell adhesion. In some cases, antibodies encoded by polynucleotides of the present invention may also inhibit monocyte adhesion through association with the Mac-1 receptor of monocytes and neutrophils.

Abciximab polynucleotides disclosed herein may be used in combination with percutaneous coronary intervention (PCI) in the prevention of complications associated with cardiac ischemic events. Such usage may be applied to subjects undergoing PCI and/or in subjects comprising unstable angina that may not be responding to typical treatment and that may be intended for PCI within about 24 hours. Further, polynucleotides of the present invention may be used with aspirin and/or heparin.

Dose and regiment of the abciximab polynucleotides disclosed herein may be adjusted to achieve optimal antibody levels and/or activity as described in the REOPRO® manufacturer's administration guide and/or the FDA REOPRO® administration guide, the contents of each of which are herein incorporated by reference in their entirety.

While contraindications associated with REOPRO® administration include internal bleeding, gastrointestinal bleeding, genitourinary bleeding, cerebrovascular accidents, bleeding diathesis, oral anticoagulants within 1 week of administration, thrombocytopenia, major surgery or trauma, intracranial neoplasm, arteriovenous malformation, aneurysm, severe uncontrolled hypertension, presumed or documented history of vasculitis, use of intravenous dextran before PCI and/or known hypersensitivity to components of the product or murine proteins, the polynucleotides on the present invention are expected to have fewer side effects and/or contraindications and as such these may be avoided by replacement with administration of polynucleotides disclosed herein.

Abciximab antibodies encoded by polynucleotides of the present invention may remain in subjects until removal, e.g. through opsonization by way of the reticuloendothelial system facilitated by association with platelets. Alternatively, removal may occur due to anti-murine antibody production. In some cases, polynucleotide-derived antibody levels may decrease rapidly, comprising a half-life of about 10 minutes or less during an initial phase, and a half-life of about 30 minutes during a second phase. Differences in half-life between phases may be due to rapid GPIIb/IIIa receptor binding.

Certain sequences encoding abciximab fragments, domains or heavy or light chains are given in Table 2. The table is not an exhaustive list and any fragment or portion of the sequence may be encoded in the abciximab polynucleotides of the invention.

TABLE 2
Table of Abciximab Sequences

| | | SEQ ID |
|---|---|---|
| Description | Sequence | NO |
| ReoPro-like | EVQLQQSGAELVKPGASVKLSCTASGFNIKDTYVH | 1 |
| antibody Heavy | WVKQRPEQGLEWIGRIDPANGYTKYDPKFQGKATI | |
| Chain | TADTSSNTAYLQLSSLTSEDTAVYYCVRPLYDYYA | |
| | MDYWGQGTSVTVSSAKTTAPSVYPLAPVCGDTTGS | |
| | SVTLGCLVKGYFPEPVTLTWNSGSLSSGVHTFPAVL | |
| | QSDLYTLSSSVTVTSSTWPSQSITCNVAHPASSTKV | |
| | DKKIEPRPKSCDKTHTCPPCPAPELLGGPSVFLFPPK | |
| | PKDTLMISRTPEVTCVVVDVSHEDPEVKFNWYVDG | |
| | VEVHNAKTKPREEQYNSTYRVVSVLTVLHQDWLN | |
| | GKEYKCKVSNKALPAPIEKTISKAKGQPREPQVYTL | |
| | PPSRDELTKNQVSLTCLVKGFYPSDIAVEWESNGQPE | |
| | NNYKTTPPVLDSDGSFFLYSKLTVDKSRWQQGNV | |
| | FSCSVMHEALHNHYTQKSLSLSPGK | |
| ReoPro-like | DILMTQSPSSMSVSLGDTVSITCHASQGISSNIGWLQ | 2 |
| antibody Light | QKPGKSFMGLIYYGTNLVDGVPSRFSGSGSGADYS | |
| Chain | LTISSLDSEDFADYYCVQYAQLPYTFGGGTKLEIKR | |
| | ADAAPTVSIFPPSSEQLTSGGASVVCFLNNFYPKDIN | |
| | VKWKIDGSERQNGVLNSWTDQDSKDSTYSMSSTLTLTK | |
| | DEYERHNSYTCEATHKTSTSPIVKSFNRNEC | |

According to the present invention, the abciximab polynucleotides of the present invention are engineered such that they may comprise one or more coding regions.

The coding regions of the abciximab polynucleotides may encode any of the regions or portions of the abciximab antibody. They may also further comprise coding regions not found in the original or parent abciximab antibody.

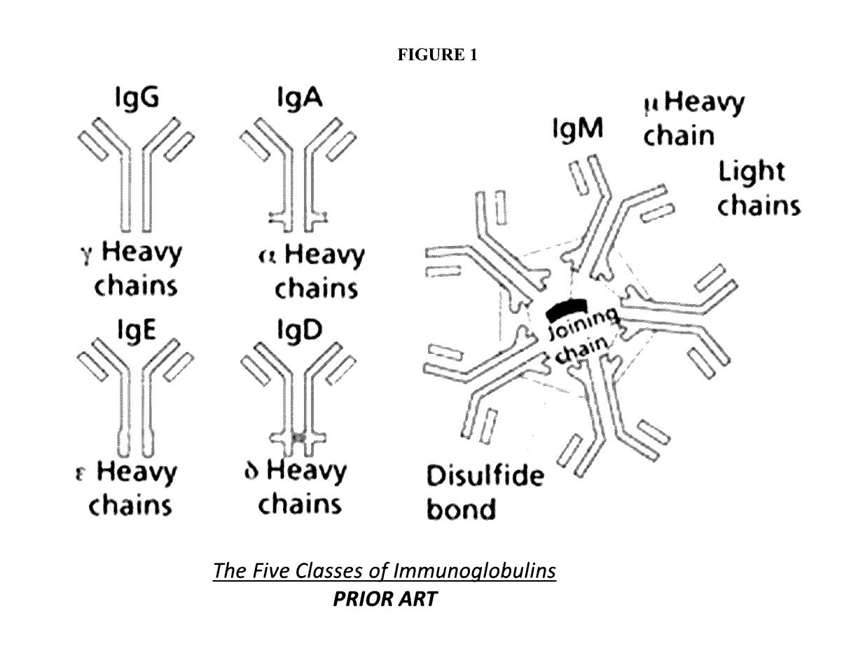

The abciximab polynucleotides of the present invention may be engineered to produce any of the 5 standard classes of immunoglobulins as shown in FIG. 1 using the abciximab antibody or any of its component parts as a starting molecule.

The abciximab polynucleotides may also be engineered according to the present invention to produce a variant abciximab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Adalimumab Parent Molecule or Antibody

According to the present invention, adalimumab polynucleotides or constructs and their associated adalimumab compositions are designed to produce the adalimumab antibody, a variant or a portion thereof in vivo.

Adalimumab, also known as HUMIRA® was derived from clone D2E7 and developed by BASF Bioresearch Corporation and Cambridge Antibody Technology and manufactured by BASF Bioresearch Corporation, Abott Laboratories.

It was first discovered using phage display technology developed by Cambridge Antibody Technology (CAT).

Adalimumab is a fully human antibody developed using phage display technology to target tumor necrosis factor α [Kempeni, J. 1999. Ann Rheum Dis. 58:(Suppl I)I70-I72]. Development began using guided selection with MAK195 mouse monoclonal antibody (Lindner, H. et al., 1997. Blood. 89(6):1931-8) as a starting molecule. Single chain variable domain fragment (scFv) libraries were constructed comprising constructs with the heavy chain variable domain (V_{H}) from MAK195 paired with human light chain variable domains (V_{L}). Once suitable binders were identified, the selected V_{L} domain was used to construct a new library where it was paired with human V_{H} domains. This led to the identification of fully human variable domains that were incorporated into a human IgG and further optimized for desired affinity and activity. Given that the resulting antibody comprises only human components, it has less potential to generate an immune response in patients that is directed to the antibody itself. Adalimumab is currently approved for treatment of a number of indications including rheumatoid arthritis, psoriasis as well as other inflammatory conditions.

In some embodiments adalimumab polynucleotides of the present invention encode polypeptides (antibodies) which comprise 1330 amino acids with a molecular weight of about 148 kDa. Such antibodies may comprise Ig gamma-1 or Ig gamma-4. In some cases, adalimumab polynucleotides may encode IgG antibodies comprising two kappa light chains (approximately 24 kDa each) and two IgG1^{z, a} heavy chains (approximately 49 kDa each) adalimumab polynucleotides may encode any of the variable domain sequences and/or one or more of the complementarity determining regions (CDRs) disclosed in U.S. Pat. No. 6,090,382, the contents of which are herein incorporated by reference in their entirety.

The adalimumab antibodies of the present invention may specifically bind TNF-α. Such binding may prevent TNF-α signaling activity that typically occurs through the interaction between TNF-α and its cell surface receptors. Such receptors may include p55 and/or p75. In some cases, cells comprising surface expressed TNF-α may be subject to lysis upon treatment with mmRNA-derived antibodies disclosed herein.

Adalimumab polynucleotides encoding antibodies of the present invention may be used to treat diseases of the immune system. Such diseases and/or conditions may include, but are not limited to rheumatoid arthritis (RA), psoriatic arthritis, ankylosing spondylitis and Crohn's disease.

Adalimumab polynucleotide encoded antibodies may comprise a half-life within subjects of from about 10 to about 20 days. Clearance rates for such antibodies may comprise about 12 ml/hour wherein subjects have been treated with a dose of about 0.25 mg/kg to about 10 mg/kg.

Adalimumab antibody therapies in the art are known to be affected by interactions with other drugs. Combination with canakinumab, rilonacept and/or tofacitinib therapy may lead to increased immunosuppressive effects and/or elevated risk of infection. When combined with trastuzumab treatment, risk of neutropenia and/or anemia may be increased. During such combined therapy, subjects may be monitored for symptoms typical of adverse effects. The adalimumab polynucleotides of the present invention are not expected to exhibit all of the foregoing side effects or contraindications.

Certain sequences encoding adalimumab fragments, domains or heavy or light chains are given in Table 3. The table is not an exhaustive list and any fragment or portion of the sequence may be encoded in the adalimumab polynucleotides of the invention.

TABLE 3
Table of Adalimumab Sequences

| Description | Sequence | SEQ ID NO |
|---|---|---|
| D2E7 Heavy | EVQLVESGGGLVQPGRSLRLSCAASGFTFDDYA | 3 |
| Chain | MHWVRQAPGKGLEWVSAITWNSGHIDYADSVE | |
| | GRFTISRDNAKNSLYLQMNSLRAEDTAVYYCAK | |
| | VSYLSTASSLDYWGQGTLVTVSSASTKGPSVFPL | |
| | APSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| D2E7 Light Chain | DIQMTQSPSSLSASVGDRVTITCRASQGIRNYLA | 4 |
| | WYQQKPGKAPKLLIYAASTLQSGVPSRFSGSGSG | |
| | TDFTLTISSLQPEDVATYYCQRYNRAPYTFGQGT | |
| | KVEIKRTVAAPSVFIFPPSDEQLKSGTASVVCLLN | |
| | NFYPREAKVQWKVDNALQSGNSQESVTEQDSK | |
| | DSTYSLSSTLTLSKADYEKHKVYACEVTHQGLSS | |
| | PVTKSFNRGEC | |
| D2E7 Heavy | EVQLVESGGGLVQPGRSLRLSCAASGFTFDDYA | 5 |
| Chain, Variable | MHWVRQAPGKGLEWVSAITWNSGHIDYADSVE | |
| domain only | GRFTISRDNAKNSLYLQMNSLRAEDTAVYYCAK | |
| | VSYLSTASSLDYWGQGTLVTVSS | |
| D2E7 Light | DIQMTQSPSSLSASVGDRVTITCRASQGIRNYLA | 6 |
| Chain, Variable | WYQQKPGKAPKLLIYAASTLQSGVPSRFSGSGSG | |
| domain only | TDFTLTISSLQPEDVATYYCQRYNRAPYTFGQGT | |
| | KVEIK | |

According to the present invention, the adalimumab polynucleotides of the present invention are engineered such that they may comprise one or more coding regions.

The coding regions of the adalimumab polynucleotides may encode any of the regions or portions of the adalimumab antibody. They may also further comprise coding regions not found in the original or parent adalimumab antibody.

The adalimumab polynucleotides of the present invention may be engineered to produce any of the 5 standard classes of immunoglobulins as shown in FIG. 1 using the adalimumab antibody or any of its component parts as a starting molecule.

The adalimumab polynucleotides may also be engineered according to the present invention to produce a variant adalimumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Alemtuzumab Parent Molecule or Antibody

According to the present invention, alemtuzumab polynucleotides or constructs and their associated alemtuzumab compositions are designed to produce the alemtuzumab antibody, a variant or a portion thereof in vivo.

Alemtuzumab also known as CAMPATH® and Campath-1H is made by Genzyme.

Graft-versus-host disease is a major issue facing bone marrow transplantation procedures. In the absence of treatment, such disease may prevent transplantation with marrow from unmatched donors and may affect 50%-70% of recipients receiving marrow from siblings that are fully matched.

Alemtuzumab is a monoclonal antibody used to reduce lymphocyte populations. It functions by binding to lymphocyte surface glycoprotein CD52, leading eventually to antibody-dependent cell-mediated cytotoxicity (ADCC.) CD52 is expressed by a number of lymphocytic cells including monocytes, macrophages and granulocytes. It is not; however, expressed by erythrocytes or hematopoetic stem cells, enabling Alemtuzumab to selectively reduce lymphocyte numbers. Therapeutic uses for alemtuzumab include reducing the number of mature T lymphocytes from donor bone marrow prior to transplanting such bone marrow in a recipient (Hale, G. et al. 1983. Blood. 62(4):873-82) as well as in the treatment of B-cell chronic lymphocytic leukemia (CLL.)

Alemtuzumab is a humanized monoclonal antibody developed by grafting complementarity determining regions (CDRs) from the rat monoclonal antibody, CAMPATH-1G, onto a human IgG1, kappa framework and is characterized extensively by Crowe et al (Crowe, J. S. et al., 1992. Clin Exp Immunol. 87, 105-10, the contents of which are herein incorporated by reference in their entirety.)

Polynucleotides of the present invention may encode campath-1H as described in Crowe, J. S. et al., 1992. Clin Exp Immunol. 87, 105-10 and/or U.S. Pat. No. 6,120,766, the contents of each of which are herein incorporated by reference in their entirety. In some cases, polynucleotides may encode one or more of the sequences listed in Table 4.

In some embodiments, polynucleotides of the present invention may be used to treat donor bone marrow prior to transplantation. Such treatment may lead to a reduction in the number of mature T lymphocytes, providing protection from graft-versus-host disease.

Polynucleotide encoding antibodies of the present invention may comprise a volume of distribution of 0.18 L/kg. The half life of such antibodies may comprise from about 100 to about 300 hours. In some cases, the half life may comprise about 288 hours.

Alemtuzumab treatment may lead to adverse effects when combined with other treatments. Combination with trastuzumab treatment may, in some cases, lead to an elevated risk for neutropenia and/or anemia. In some cases, substitute treatment with polynucleotides of the present invention may avoid such adverse effects.

Certain sequences encoding alemtuzumab fragments, domains or heavy or light chains are given in Table 4. The table is not an exhaustive list and any fragment or portion of the sequence may be encoded in the alemtuzumab polynucleotides of the invention.

TABLE 4
Table of Alemtuzumab Sequences

| | | SEQ |
|---|---|---|
| | | ID |
| Description | Sequence | NO |
| CAMPATH- | QVQLQESGPGLVRPSQTLSLTCTVSGFTFTDFYMNWVR | 7 |
| 1H:Heavy Chain 1 | QPPGRGLEWIGFIRDKAKGYTTEYNPSVKGRVTMLVDT | |
| | SKNQFSLRLSSVTAADTAVYYCAREGHTAAPFDYWGQ | |
| | GSLVTVSSASTKGPSVFPLAPSSKSTSGGTAALGCLVKD | |
| | YFPEPVTVSWNSGALTSGVHTFPAVLQSSGLYSLSSVV | |
| | TVPSSSLGTQTYICNVNHKPSNTKVDKKVEPKSCDKTH | |
| | TCPPCPAPELLGGPSVFLFPPKPKDTLMISRTPEVTCVVV | |
| | DVSHEDPEVKFNWYVDGVEVHNAKTKPREEQYNSTY | |
| | RVVSVLTVLHQDWLNGKEYKCKVSNKALPAPIEKTISK | |
| | AKGQPREPQVYTLPPSRDELTKNQVSLTCLVKGFYPSDI | |
| | AVEWESNGQPENNYKTTPPVLDSDGSFFLYSKLTVDKS | |
| | RWQQGNVFSCSVMHEALHNHYTQKSLSLSPGK | |
| CAMPATH- | DIQMTQSPSSLSASVGDRVTITCKASQNIDKYLNWYQQ | 8 |
| 1H:Light Chain 1 | KPGKAPKLLIYNTNNLQTGVPSRFSGSGSGTDFTFTISSL | |
| | QPEDIATYYCLQHISRPRTFGQGTKVEIKRTVAAPSVFIF | |
| | PPSDEQLKSGTASVVCLLNNFYPREAKVQWKVDNALQ | |
| | SGNSQESVTEQDSKDSTYSLSSTLTLSKADYEKHKVYA | |
| | CEVTHQGLSSPVTKSFNR | |
| CAMPATH-1H | QVQLQESGPGLVRPSQTLSLTCTVSGFTFTDFYMNWVR | 9 |
| Humanized | QPPGRGLEWIGFIRDKAKGYTTEYNPSVKGRVTMLVDT | |
| VH-CH1 (VH(1-121) + | SKNQFSLRLSSVTAADTAVYYCAREGHTAAPFDYWGQ | |
| CH1(122-210)) | GSLVTVSSASTKGPSVFPLAPSSKSTSGGTAALGCLVKD | |
| | YFPEPVTVSWNSGALTSGVHTFPAVLQSSGLYSLSSVV | |
| | TVPSSSLGTQTYICNVNHKPSNTKVDKKV | |
| CAMPATH-1H | DIQMTQSPSSLSASVGDRVTITCKASQNIDKYLNWYQQ | 10 |
| Humanized | KPGKAPKLLIYNTNNLQTGVPSRFSGSGSGTDFTFTISSL | |
| L-KAPPA (V- | QPEDIATYYCLQHISRPRTFGQGTKVEIKRTVAAPSVFIF | |
| KAPPA(1-107) + | PPSDEQLKSGTASVVCLLNNFYPREAKVQWKVDNALQ | |
| C- | SGNSQESVTEQDSKDSTYSLSSTLTLSKADYEKHKVYA | |
| KAPPA(108-214)) | CEVTHQGLSSPVTKSFNRGEC | |
| alemtuzumab | QVQLQESGPGLVRPSQTLSLTCTVSGFTFTDFYMNWVR | 11 |
| H-GAMMA-1 | QPPGRGLEWIGFIRDKAKGYTTEYNPSVKGRVTMLVDT | |
| (VH(1-121) + | SKNQFSLRLSSVTAADTAVYYCAREGHTAAPFDYWGQ | |
| CH1(122-219) + | GSLVTVSSASTKGPSVFPLAPSSKSTSGGTAALGCLVKD | |
| HINGE- | YFPEPVTVSWNSGALTSGVHTFPAVLQSSGLYSLSSVV | |
| REGION(220-220) + | TVPSSSLGTQTYICNVNHKPSNTKVDKKVEAPELLGGP | |
| CH2(221-330) + | SVFLFPPKPKDTLMISRTPEVTCVVVDVSHEDPEVKFN | |
| CH3(331-437) | WYVDGVEVHNAKTKPREEQYNSTYRVVSVLTVLHQD | |
| | WLNGKEYKCKVSNKALPAPIEKTISKAKGQPREPQVYT | |
| | LPPSRDELTKNQVSLTCLVKGFYPSDIAVEWESNGQPE | |
| | NNYKTTPPVLDSDGSFFLYSKLTVDKSRWQQGNVFSCS | |
| | VMHEALHNHYTQKSLSLSPGK | |
| alemtuzumab | DIQMTQSPSSLSASVGDRVTITCKASQNIDKYLNWYQQ | 12 |
| L-KAPPA (V- | KPGKAPKLLIYNTNNLQTGVPSRFSGSGSGTDFTFTISSL | |
| KAPPA(1-107) + | QPEDIATYYCLQHISRPRTFGQGTKVEIKRTVAAPSVFIF | |
| C- | PPSDEQLKSGTASVVCLLNNFYPREAKVQWKVDNALQ | |
| KAPPA(108-214)) | SGNSQESVTEQDSKDSTYSLSSTLTLSKADYEKHKVYA | |
| | CEVTHQGLSSPVTKSFNRGEC | |
| CAMPATH-1H | QVQLQESGPGLVRPSQTLSLTCTVSGFTFTDFYMNWVR | 13 |
| Humanized | QPPGRGLEWIGFIRDKAKGYTTEYNPSVKGRVTMLVDT | |
| VH-CH1 (VH(1-121) + | SKNQFSLRLSSVTAADTAVYYCAREGHTAAPFDYWGQ | |
| CH1(122-219)) | GSLVTVSSASTKGPSVFPLAPSSKSTSGGTAALGCLVKD | |
| | YFPEPVTVSWNSGALTSGVHTFPAVLQSSGLYSLSSVV | |
| | TVPSSSLGTQTYICNVNHKPSNTKVDKKVE | |
| CAMPATH-1H | DIQMTQSPSSLSASVGDRVTITCKASQNIDKYLNWYQQ | 14 |
| Humanized | KPGKAPKLLIYNTNNLQTGVPSRFSGSGSGTDFTFTISSL | |
| L-KAPPA (V- | QPEDIATYYCLQHISRPRTFGQGTKVEIKRTVAAPSVFIF | |
| KAPPA(1-107) + | PPSDEQLKSGTASVVCLLNNFYPREAKVQWKVDNALQ | |
| C- | SGNSQESVTEQDSKDSTYSLSSTLTLSKADYEKHKVYA | |
| KAPPA(108-211)) | CEVTHQGLSSPVTKSFNR | |

According to the present invention, the alemtuzumab polynucleotides of the present invention are engineered such that they may comprise one or more coding regions.

The coding regions of the alemtuzumab polynucleotides may encode any of the regions or portions of the alemtuzumab antibody. They may also further comprise coding regions not found in the original or parent alemtuzumab antibody.

The alemtuzumab polynucleotides of the present invention may be engineered to produce any of the 5 standard classes of immunoglobulins as shown in FIG. 1 using the alemtuzumab antibody or any of its component parts as a starting molecule.

The alemtuzumab polynucleotides may also be engineered according to the present invention to produce a variant alemtuzumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Basiliximab Parent Molecule or Antibody

According to the present invention, basiliximab polynucleotides or constructs and their associated basiliximab compositions are designed to produce the basiliximab antibody, a variant or a portion thereof in vivo.

Basiliximab also known as SIMULECT® is a chimeric mouse-human anti-CD25 antibody. It is similar to another antibody, daclizumab.Basiliximab is chimeric version of RFT5, whereas daclizumab is humanized version of RFT5. The antibody is sold by Novartis Pharmaceuticals.

Basiliximab is a chimeric monoclonal antibody (human and mouse components) that targets CD25 on T cells. CD25 is also known as interleukin (IL)-2 receptor alpha chain and it is expressed by activated T cells. Treatment with basiliximab may be carried out to prevent rejection of transplanted organs and/or tissues by reducing T cell populations in the transplanted organs and/or tissues.

Basiliximab was developed using the variable domains of the mouse monoclonal anti-CD25 antibody, RFT5, and expressing them with human heavy and light chains, resulting in a chimeric antibody (mouse-human) as described in U.S. Pat. No. 6,383,487, the contents of which are herein incorporated by reference in their entirety.

In some embodiments, antibodies encoded by polynucleotides of the present invention are about 144 kDa. Such polynucleotides may encode sequences from heavy and light chain constant domains derived from human IgG1 and variable domains derived from mouse anti-CD25 antibody RFT5 as disclosed in U.S. Pat. No. 6,383,487, the contents of which are herein incorporated by reference in their entirety. In some cases, polynucleotides of the present invention may encode one or more of the amino acid sequences presented in Table 5.

Antibodies encoded by polynucleotides of the present invention may inhibit IL-2-mediated lymphocyte activation. Such inhibition may be carried out through direct binding with the CD25 alpha subunit, preventing IL-2 from binding. Such inhibition may prevent immune attack of a foreign object, including organs such as kidneys that have been transplanted.

In some embodiments, polynucleotides of the present invention may be used to treat kidneys prior to transplantation. Such treatment reduce or eliminate T cells from donor organs, thereby slowing or preventing immune rejection of such organs by a recipient.

In some cases, combining basiliximab treatment with other therapeutics may result in additive and/or adverse effects (see basiliximab FDA label, the contents of which is herein incorporated by reference in its entirety.) Combined therapy with canakinumab and/or rilonacept may increase the immunosuppressive effects of basiliximab. In some cases, this may lead to elevated risk of infection. Combination with trastuzumab treatment may, in some cases, may lead to an elevated risk for neutropenia and/or anemia. Treatment using polynucleotides of the present invention may be used in place of basiliximab treatment in order to prevent adverse effects associated with interactions between basiliximab and other treatments.

In some cases, treatment with basiliximab can lead to a variety of side effects (see the basiliximab FDA label, the contents of which is herein incorporated by reference in its entirety.) Treatment using polynucleotides of the present invention may be used in place of basiliximab treatment in order to prevent such side effects.

Antibodies encoded by polynucleotides of the present invention may comprise half lives that range from about 4 days to about 7 days. Clearance of such antibodies may occur at differing rates depending on subject age. For example, clearance may occur in adult and adolescent subjects at a rate of from about 30 ml/h to about 45 ml/h. In pediatric subjects, clearance may be more rapid with rates of from about 15 ml/h to about 20 ml/h.

Certain sequences encoding basiliximab fragments, domains or heavy or light chains are given in Table 5. The table is not an exhaustive list and any fragment or portion of the sequence may be encoded in the basiliximab polynucleotides of the invention.

TABLE 5
Table of Basiliximab Sequences

| Description | Sequence | SEQ ID NO |
|---|---|---|
| Anti-CD25 antibody | QLQQSGTVLARPGASVKMSCKASGYSFTRYWM | 15 |
| heavy CHIMERIC | HWIKQRPGQGLEWIGAIYPGNSDTSYNQKFEGK | |
| chain | AKLTAVTSASTAYMELSSLTHEDSAVYYCSRDY | |
| | GYYFDFWGQGTTLTVSSASTKGPSVFPLAPSSK | |
| | STSGGTAALGCLVKDYFPEPVTVSWNSGALTSG | |
| | VHTFPAVLQSSGLYSLSSVVTVPSSSLGTQTYIC | |
| | NVNHKPSNTKVDKRVEPPKSCDKTHTCPPCPAP | |
| | ELLGGPSVFLFPPKPKDTLMISRTPEVTCVVVDV | |
| | SHEDPEVKFNWYVDGVEVHNAKTKPREEQYNS | |
| | TYRVVSVLTVLHQDWLNGKEYKCKVSNKALP | |
| | APIEKTISKAKGQPREPQVYTLPPSRDELTKNQV | |
| | SLTCLVKGFYPSDIAVEWESNGQPENNYKTTPP | |
| | VLDSDGSFFLYSKLTVDKSRWQQGNVFSCSVM | |
| | HEALHNHYTQKSLSLSPGK | |
| Anti-CD25 antibody | QIVSTQSPAIMSASPGEKVTMTCSASSSRSYMQ | 16 |
| light CHIMERIC | WYQQKPGTSPKRWIYDTSKLASGVPARFSGSGS | |
| chain | GTSYSLTISSMEAEDAATYYCHQRSSYTFGGGT | |
| | KLEIKRTVAAPSVFIFPPSDEQLKSGTASVVCLL | |
| | NNFYPREAKVQWKVDNALQSGNSQESVTEQDS | |
| | KDSTYSLSSTLTLSKADYEKHKVYACEVTHQGL | |
| | SSPVTKSFNRGE | |

According to the present invention, the basiliximab polynucleotides of the present invention are engineered such that they may comprise one or more coding regions.

The coding regions of the basiliximab polynucleotides may encode any of the regions or portions of the basiliximab antibody. They may also further comprise coding regions not found in the original or parent basiliximab antibody.

The basiliximab polynucleotides of the present invention may be engineered to produce any of the 5 standard classes of immunoglobulins as shown in FIG. 1 using the basiliximab antibody or any of its component parts as a starting molecule.

The basiliximab polynucleotides may also be engineered according to the present invention to produce a variant basiliximab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Belimumab Parent Molecule or Antibody

According to the present invention, belimumab polynucleotides or constructs and their associated belimumab compositions are designed to produce the belimumab antibody, a variant or a portion thereof in vivo.

Belimumab also known as BENLYSTA®, LYMPHOSTAT-B® and BmAb was initially developed by Human Genome Sciences and MedImmune and licensed to GlaxoSmithKline.

Belimumab is a fully human antibody that has been developed to treat autoimmune disorders. Belimumab targets B-lymphocyte stimulator (BLyS,) a member of the TNF family of signaling molecules (Drugs R D 2010; 10(1):55-65.) Binding prevents maturation of B-lymphocytes into mature B-cells.

Using phage libraries, human single chain variable fragment (scFv) repertoires were developed and screened for candidates with high affinity for BLyS (Baker, K. P. et al., 2003. Arthritis Rheum. 48(11):3253-65.) High affinity candidates were further optimized to develop scFvs with optimal binding. Lead candidates were expressed with human IgG constant domains to produce fully human antibodies that were subjected to further characterization and selection for the ability to block BLyS signaling activity. A lead candidate, LymphoStat-B was identified and used to develop belimumab.

In some embodiments, polynucleotides of the present invention may encode a fully human antibody capable of binding and blocking the signal transduction of BLyS such as any of the antibodies described by Baker et al (Baker, K. P. et al., 2003. Arthritis Rheum. 48(11):3253-65, the contents of which are herein incorporated by reference in their entirety.) In some cases, polynucleotides encode one or more variable domain portions of scFvs claimed in U.S. Pat. Nos. 7,138,501 and/or 7,605,236, the contents of each of which are herein incorporated by reference in their entirety. In some cases, polynucleotides may encode one or more components of one or more antibodies disclosed in any of U.S. Pat. Nos. 7,138,501, 7,605,236, 7,879,328, 8,062,906, 8,071,092, 8,101,181, 8,173,122, 8,231,873 and/or 8,303,951, the contents of each of which are herein incorporated by reference in their entirety. Further, polynucleotides of the present invention may encode one or more of any of the sequences listed in Table 6.

Polynucleotides of the present invention may encode one or more antibodies that bind BLyS and prevent signaling activity associated with it.

Polynucleotides of the present invention may be used in the treatment of a number of diseases and/or conditions. Such diseases and/or conditions may include, but are not limited to systemic lupus erythematosus and rheumatoid arthritis.

Belimumab treatment may lead to adverse effects when combined with other treatments. In some cases, combined treatment with belatacept, denosumab, fingolimod and/or hydroxyurea may lead to elevation of immunosuppressive effects. Additionally, combination with ado-trastuzumab emtansine and/or golimumab may increase the risk of developing one or more side effects associated with belimumab treatment. In some embodiments, treatment with polynucleotides of the present invention in place of belimumab treatment may prevent adverse interactions with these other treatments.

In some cases, antibodies encoded by polynucleotides of the present invention may comprise a clearance rate in serum of from about 2 ml/day/kg to about 6 ml/day/kg.

Certain sequences encoding belimumab fragments, domains or heavy or light chains are given in Table 6. The table is not an exhaustive list and any fragment or portion of the sequence may be encoded in the belimumab polynucleotides of the invention. In the table, the underlined region indicates an example of a linker between VH and VL domains in an scFv.

TABLE 6
Table of Belimumab Sequences

| | | SEQ ID |
|---|---|---|
| Description | Sequence | NO. |
| scFv (SEQ ID | QVQLQQSGAEVKKPGSSVRVSCKASGGTFNNN | 17 |
| NO: 327) claimed | AINWVRQAPGQGLEWMGGIIPMFGTAKYSQNF | |
| in Patent No. U.S. Pat. No. | QGRVAITADESTGTASMELSSLRSEDTAVYYC | |
| 7,138,501 | ARSRDLLLFPHHALSPWGRGTMVTVSSGGGGS | |
| | GGGGSGGGGSAFSSELTQDPAVSVALGQTVRV | |
| | TCQGDSLRSYYASWYQQKPGQAPVLVIYGKN | |
| | NRPSGIPDRFSGSSSGNTASLTITGAQAEDEADY | |
| | YCSSRDSSGNHWVFGGGTELTVLG | |
| VH of scFv (SEQ | QVQLQQSGAEVKKPGSSVRVSCKASGGTFNNN | 18 |
| ID NO 327) | AINWVRQAPGQGLEWMGGIIPMFGTAKYSQNF | |
| claimed in | QGRVAITADESTGTASMELSSLRSEDTAVYYC | |
| U.S. Pat. No. 7,138,501 | ARSRDLLLFPHHALSPWGRGTMVTVSS | |
| VL of scFv (SEQ | SSELTQDPAVSVALGQTVRVTCQGDSLRSYYA | 19 |
| ID NO 327) | SWYQQKPGQAPVLVIYGKNNRPSGIPDRFSGSS | |
| claimed in | SGNTASLTITGAQAEDEADYYCSSRDSSGNHW | |
| U.S. Pat. No. 7,138,501 | VFGGGTELTVLG | |
| 1^{st} scFv of claim 1 | QVQLQQWGAGLLKPSETLSLTCAVYGGSFSGY | 20 |
| in patent number | YWSWIRQPPGKGLEWIGEINHSGSTNYNPSLKS | |
| U.S. Pat. No. 7,605,236 | RVTISVDTSKNQFSLKLSSVTAADTAVYYCAR | |
| | GPRYYDILTGYRYNWFDPWGRGTLVTVSSGG | |
| | GGSGGGGSGGGGSDIVMTQSPSTLSASVGDRV | |
| | TITCRASQGISSWLAWYQQKPGRAPKVLIYKAS | |
| | TLESGVPSRFSGSGSGTDFTLTISSLQPEDFATY | |
| | YCQQSYSTPWTFGQGTKLEIKR | |
| VH of scFv | QVQLQQWGAGLLKPSETLSLTCAVYGGSFSGY | 21 |
| claimed in patent | YWSWIRQPPGKGLEWIGEINHSGSTNYNPSLKS | |
| number | RVTISVDTSKNQFSLKLSSVTAADTAVYYCAR | |
| U.S. Pat. No. 7,605,236 | GPRYYDILTGYRYNWFDPWGRGTLVTVSS | |
| 2^{nd} scFv of claim 1 | QLQLQESGPGLVKPSETLSLTCTVSGGFISSRTS | 22 |
| in patent number | YWGWIRQPPGKGPEWIGNIYYTGKTYYSPSLK | |
| U.S. Pat. No. 7,605,236 | SRVTISVDTSKNQLSLKLNSVTAADTAVYYCA | |
| | RAGYDLLTGYPFYFDSWGKGTLVTVSSGGGGS | |
| | GGGGSGGGGSALEIVLTQSPATLSLSPGERATL | |
| | SCRASQSVSSYLAWYQQKPGQAPRLLIYDASN | |
| | RATGIPARFSGSGSGTDFTLTISSLEPEDFAVYY | |
| | CQQRSNWPFLTFGGGTKVEIKR | |
| Light chain | EIVLTQSPATLSLSPGERATLSCRASQSVSSYLA | 23 |
| variable domain of | WYQQKPGQAPRLLIYDASNRATGIPARFSGSGS | |
| 2^{nd} scFv claimed | GTDFTLTISSLEPEDFAVYYCQQRSNWPFLTFG | |
| in patent No | GGTKVEIKR | |
| U.S. Pat. No. 7,605,236 | | |

According to the present invention, the belimumab polynucleotides of the present invention are engineered such that they may comprise one or more coding regions.

The coding regions of the belimumab polynucleotides may encode any of the regions or portions of the belimumab antibody. They may also further comprise coding regions not found in the original or parent belimumab antibody.

The belimumab polynucleotides of the present invention may be engineered to produce any of the 5 standard classes of immunoglobulins as shown in FIG. 1 using the belimumab antibody or any of its component parts as a starting molecule.

The belimumab polynucleotides may also be engineered according to the present invention to produce a variant belimumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Bevacizumab Parent Molecule or Antibody

According to the present invention, bevacizumab polynucleotides or constructs and their associated bevacizumab compositions are designed to produce the bevacizumab antibody, a variant or a portion thereof in vivo.

Bevacizumab, also known as AVASTIN® is manufactured by Roche Pharmaceuticals.

Bevacizumab is a 149 kDa humanized monoclonal IgG1 directed against human vascular endothelial growth factor (VEGF.) While the antibody framework comprises human components, the complimentarily-determining regions (CDRs) are derived from a murine anti-VEGF antibody A.4.6.1 (Presta et al. 1997. Cancer Res. 57(20):4593-9 and FDA label.) In preclinical models, bevacizumab has been tested for the treatment of a variety of cancer types and in combination with other cancer treatments (Chen, H. X. 2004. The Oncologist. Vol. 9, Suppl 1:27-35.)

Polynucleotides of the present invention may encode one or more of any of the heavy chain and/or light chain amino acid sequences, one or more of any of the variable and/or constant domain amino acid sequences and/or one or more of any of the CDR amino acid sequences disclosed in U.S. Pat. No. 7,060,269 and/or International Publication Nos. WO1994/010202A1, WO1996/030046A1 and WO1998/045332A2, the contents of each of which are herein incorporated by reference in their entirety.

In some cases, such encoded antibodies may comprise a K_{d} value that does not exceed about 1×10⁻⁸M. In some cases, antibodies comprise human antibody framework residues and murine-derived CDR residues. In further embodiments, some human framework residues are also replaced with murine framework residues. Such antibodies may comprise amino acid sequences that correspond with humanized antibodies and/or variable light and/or heavy domains taught by Presta et al (Presta et al. 1997. Cancer Res. 57(20):4593-9.)

The polynucleotide encoded antibodies disclosed herein may bind VEGF and inhibit VEGF activity by preventing interaction between VEGF and its receptors (including, but not limited to Flt-1 and KDR.) Such inhibition may prevent the growth and/or proliferation of vascular endothelial cells and lead to a reduction in the formation of blood vessels and or a reduction in the number of blood vessels. In the treatment of metastatic disease, the reduction of blood vessel number and/or formation may reduce the nutrient delivery to cancerous cells, thereby reducing the number of such cells and/or killing them.

The polynucleotides of the present invention may be used to treat multiple forms of cancer. Such cancers may include, but are not limited to metastatic kidney cancer, glioblastoma, non-small cell lung cancer, colorectal cancer and HER2-negative breast cancer. In some cases, they may be used to treat one or more tumors demonstrated to overexpress VEGF, such tumors including any of those indicated by Herbst et al. (Herbst et al. 2004. Oncologist. 9 Suppl 1:19-26.)

In some cases, the polynucleotides may be combined with one or more other therapies to improve one or more therapeutic outcome. Combination with chemotherapy may promote longer times to progression and/or greater survival in subjects with cancer than with chemotherapy alone.

Contraindications may comprise combined treatment with sunitinib. Such combined treatment may elevate the occurrence of adverse effects associated with bevacizumab and/or sunitinib treatment including, but not limited to hypertension, anemia and/or microangiopathic hemolytic anemia. It is expected that the polynucleotides of the present invention may overcomes some or all of these contraindications.

Certain sequences encoding bevacizumab fragments, domains or heavy or light chains are given in Table 7. The table is not an exhaustive list and any fragment or portion of the sequence may be encoded in the bevacizumab polynucleotides of the invention.

TABLE 7
Table of Bevacizumab Sequences

| Description | Sequence | SEQ ID NO. |
|---|---|---|
| Bevacizumab | EVQLVESGGGLVQPGGSLRLSCAASGYTFTNYGMN | 24 |
| heavy chain | WVRQAPGKGLEWVGWINTYTGEPTYAADFKRRFTF | |
| | SLDTSKSTAYLQMNSLRAEDTAVYYCAKYPHYYGS | |
| | SHWYFDVWGQGTLVTVSSASTKGPSVFPLAPSSKST | |
| | SGGTAALGCLVKDYFPEPVTVSWNSGALTSGVHTFP | |
| | AVLQSSGLYSLSSVVTVPSSSLGTQTYICNVNHKPSN | |
| | TKVDKKVEPKSCDKTHTCPPCPAPELLGGPSVFLFPP | |
| | KPKDTLMISRTPEVTCVVVDVSHEDPEVKFNWYVD | |
| | GVEVHNAKTKPREEQYNSTYRVVSVLTVLHQDWLN | |
| | GKEYKCKVSNKALPAPIEKTISKAKGQPREPQVYTLP | |
| | PSREEMTKNQVSLTCLVKGFYPSDIAVEWESNGQPE | |
| | NNYKTTPPVLDSDGSFFLYSKLTVDKSRWQQGNVFS | |
| | CSVMHEALHNHYTQKSLSLSPGK | |
| Bevacizumab | DIQMTQSPSSLSASVGDRVTITCSASQDISNYLNWYQ | 25 |
| light chain | QKPGKAPKVLIYFTSSLHSGVPSRFSGSGSGTDFTLTI | |
| | SSLQPEDFATYYCQQYSTVPWTFGQGTKVEIKRTVA | |
| | APSVFIFPPSDEQLKSGTASVVCLLNNFYPREAKVQW | |
| | KVDNALQSGNSQESVTEEDSKDSTYSLSSTLTLSKAD | |
| | YEKHKVYACEVTHQGLSSPVTKSFNRGEC | |

According to the present invention, the bevacizumab polynucleotides of the present invention are engineered such that they may comprise one or more coding regions.

The coding regions of the bevacizumab polynucleotides may encode any of the regions or portions of the bevacizumab antibody. They may also further comprise coding regions not found in the original or parent bevacizumab antibody.

The bevacizumab polynucleotides of the present invention may be engineered to produce any of the 5 standard classes of immunoglobulins as shown in FIG. 1 using the bevacizumab antibody or any of its component parts as a starting molecule.

The bevacizumab polynucleotides may also be engineered according to the present invention to produce a variant bevacizumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

Brentuximab vedontin Parent Molecule or Antibody

According to the present invention, brentuximab vedontin polynucleotides or constructs and their associated brentuximab vedontin compositions are designed to produce the brentuximab vedontin antibody, a variant or a portion thereof in vivo.

Brentuximab vedontin also known as ADCETRIS®, AC10-vcMMAE Or SGN-35 is an antineoplastic agent comprising an anti-CD30 antibody conjugated with monomethyl auristatin (MMAE) by a protease-susceptible linker. It is designed to disrupt cell cycle progression by compromising the microtubule network formation in the cytosol. This prevents cells from entering mitosis at the end of the second gap phase. Brentuximab vedontin is able to bind tumor cells expressing CD30, leading to internalization and cleavage of the linker. Linker cleavage allows for release of MMAE which binds to components of the microtubule network leading to disruption.

The identification and utilization of CD30 as a marker and immunotherapy target was carried out during the 1980s and 1990s leading to the development of anti-CD30 monoclonal antibodies, mostly generated using purified CD30 as an immunogen or Hodgkin's disease (HD) cell lines (see U.S. Pat. No. 7,090,843.) Anti-CD30 antibody AC10, first described by Bowen et al (Bowen et al., 1993, J Immunol. 151:5896-906,) was generated using an immunogen comprising CD30 from a human cell line, YT that more closely resembled natural killer cells. AC10 was shown to be capable of arresting growth in CD30 expressing cells leading to attempts to modify it for clinical use. Wahl et al created a chimeric antibody, referred to as SGN-30 or cAC10, by cloning the variable domains of AC10 into an expression construct encoding human IgG1 heavy and light chain constant domains (Wahl, A. F. et al., 2002. Cancer Res. 62(13):3736-42.) These antibodies were shown to be capable of inducing apoptosis in HD cells. Further optimization by Francisco et al led to the drug conjugated form of the antibody embodied by Bretuximab vedontin (Francisco, J. A. et al., 2003. Blood. 102:1458-65.)

Polynucleotides encoding antibodies of the present invention may be used for the generation of antibody-drug conjugates (ADCs.) Such ADCs may be useful for delivery of payloads, including cytotoxic payloads. They may be used to target payloads to cells expressing CD30, including, but not limited to cancerous cells. Binding of such antibodies to CD30 on cell surfaces may lead to internalization of bound antibodies with or without conjugated payloads.

Polynucleotide encoded antibodies disclosed herein may comprise one or more components of one or more antibodies disclosed in U.S. Pat. No. 7,090,843 or 8,257,706 or International Publication No. WO 2005/001038, the contents of each of which are herein incorporated by reference in their entirety. Such components may include, but are not limited to any heavy chain, light chain, variable domain, constant domain and/or any complementarity-determining regions (CDRs.) Amino acid and/or nucleotide sequences for these components may include any of those listed in Table 1 of U.S. Pat. No. 7,090,843.

Polynucleotides of the present invention may be used to treat multiple forms of cancer. Such forms of cancer may include, but are not limited to Hodgkin's lymphoma and anaplastic large cell lymphoma.

Non-Hodgkin's lymphoma (NW) refers to any of a large group of cancers of lymphocytes (white blood cells). Non-Hodgkin lymphomas can occur at any age and are often marked by lymph nodes that are larger than normal, fever, and weight loss. There are many different types of non-Hodgkin lymphoma. These types can be divided into aggressive (fast-growing) and indolent (slow-growing) types, and they can be formed from either B-cells or T-cells. B-cell non-Hodgkin lymphomas include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. T-cell non-Hodgkin lymphomas include mycosis fungoides, anaplastic large cell lymphoma, and precursor T-lymphoblastic lymphoma. Lymphomas that occur after bone marrow or stem cell transplantation are usually B-cell non-Hodgkin lymphomas. Prognosis and treatment depend on the stage and type of disease.

Polynucleotides of the present invention may avoid one or more of the contraindications, side effects or adverse drug reactions associated with brentuximab vedontin (see the FDA label for contraindications, the contents of which are herein incorporated by reference in their entirety.)

Certain sequences encoding brentuximab vedontin fragments, domains or heavy or light chains are given in Table 8. The table is not an exhaustive list and any fragment or portion of the sequence may be encoded in the brentuximab vedontin polynucleotides of the invention.

TABLE 8
Table of Brentuximab Sequences

| | | SEQ |
|---|---|---|
| | | ID |
| Description | Sequence | NO. |
| L1 AC10 VL | DIVLTQSPATLSLSPGERATLSCRASQSVDFDGDS | 26 |
| domain | YMNWYQQKPGQPPKVLIYAASNLESGIPARFSGS | |
| | GSGTDFTLTISSLQPEDFATYYCQQSNEDPWTFGG | |
| | GTKVEIK | |
| L2 AC10 VL | DIVLTQSPSSLSASVGDRVTITCRASQSVDFDGDS | 27 |
| domain | YMNWYQQKPGQPPKVLIYAASNLESGIPARFSGS | |
| | GSGTDFTLTISSLQPEDFATYYCQQSNEDPWTFGG | |
| | GTKVEIK | |
| L3 AC10 VL | DIVLTQSPDSLAVSLGERATINCKASQSVDFDGDS | 28 |
| domain | YMNWYQQKPGQPPKVLIYAASNLESGIPARFSGS | |
| | GSGTDFTLTINSLEAEDAATYYCQQSNEDPWTFG | |
| | GGTKVEIK | |
| H1 AC10 VH | QIQLQQSGPEVVKPGASVKISCKASGYTFTDYYIT | 29 |
| domain | WVKQKPGQGLEWIGWIYPGSGNTKYNEKFKGKA | |
| | TLTVDTSSSTAFMQLSSLTSEDTAVYFCANYGNY | |
| | WFAYWGQGTQVTVSA | |
| H2 AC10 VH | QIQLVESGGGLVKPGGSLRLSCAASGYTFTDYYIT | 30 |
| domain | WVRQAPGQGLEWMGWIYPGSGNTKYNEKFQGR | |
| | VTMTVDTSTSTAYMELSSLRSEDTAVYFCANYGN | |
| | YWFAYWGQGTLVTVSS | |
| H3 AC10 VH | QIQLVQSGPEVKKPGASVKVSCKASGYTFTDYYI | 31 |
| domain | TWVRQAPGQGLEWMGWIYPGSGNTKYNEKFQG | |
| | RFVFSVDTSASTAYLQISSLKAEDTAVYFCANYG | |
| | NYWFAYWGQGTLVTVSS | |
| Ig gamma- | ASTKGPSVFPLAPSSKSTSGGTAALGCLVKDYFPE | 32 |
| 1 chain | PVTVSWNSGALTSGVHTFPAVLQSSGLYSLSSVV | |
| C region | TVPSSSLGTQTYICNVNHKPSNTKVDKKVEPKSC | |
| | DKTHTCPPCPAPELLGGPSVFLFPPKPKDTLMISRT | |
| | PEVTCVVVDVSHEDPEVKFNWYVDGVEVHNAKT | |
| | KPREEQYNSTYRVVSVLTVLHQDWLNGKEYKCK | |
| | VSNKALPAPIEKTISKAKGQPREPQVYTLPPSRDE | |
| | LTKNQVSLTCLVKGFYPSDIAVEWESNGQPENNY | |
| | KTTPPVLDSDGSFFLYSKLTVDKSRWQQGNVFSC | |
| | SVMHEALHNHYTQKSLSLSPGK | |

According to the present invention, the brentuximab vedontin polynucleotides of the present invention are engineered such that they may comprise one or more coding regions.

The coding regions of the brentuximab vedontin polynucleotides may encode any of the regions or portions of the brentuximab vedontin antibody. They may also further comprise coding regions not found in the original or parent brentuximab vedontin antibody.

The brentuximab vedontin polynucleotides of the present invention may be engineered to produce any of the 5 standard classes of immunoglobulins as shown in FIG. 1 using the brentuximab vedontin antibody or any of its component parts as a starting molecule.

The brentuximab vedontin polynucleotides may also be engineered according to the present invention to produce a variant brentuximab vedontin antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Canakinumab Parent Molecule or Antibody

According to the present invention, canakinumab polynucleotides or constructs and their associated canakinumab compositions are designed to produce the canakinumab antibody, a variant or a portion thereof in vivo.

Canakinumab also known as ILARIS® or ACZ-885 is a fully human monoclonal antibody (IgG1/κ isotype subclass) directed against interleukin (IL)-1β, and capable of blocking IL-1β signal transduction without interfering with other IL-1 family member signaling (Lachmann H. J. et al. 2009. N Engl J Med. 360(23):2416-25.)

Canakinumab was generated by immunizing mice with human IL-1β(Alten H. et al. 2008. Arthritis Research & Therapy. 10:R67.) These mice were genetically engineered to comprise a portion of the human immunoglobulin repertoire. Hybridomas were generated from these immunized mice and clones were selected based on the production of high affinity antibodies for IL-1β. ACZ885 produced by one of these clones was found to have an affinity for IL-1β of 40 pmol/L with no cross-reactivity for human IL-1α or murine IL-1β. In studies using human fibroblasts, ACZ885 treatment was found to block IL-1β signaling activity with IC50 of 44.6 pmol/L (7.1±0.56 ng/ml; n=6.)

Polynucleotide encoded antibodies of the present invention may comprise human heavy and light chains. Such antibodies may comprise any of the heavy and/or light chain sequences disclosed in U.S. Pat. Nos. 7,446,175 and/or 8,105,587, the contents of each or which are herein incorporated by reference in their entirety. Further, polynucleotide encoded antibodies disclosed herein may comprise one or more of the CDR amino acid sequences disclosed in those patents.

Polynucleotide encoded antibodies of the present invention may bind IL-1β and prevent IL-1β signal transduction. Such binding may comprise high affinity binding with a K_{D} of from about 25 to about 35 pM.

Rheumatoid arthritis (RA) is a chronic autoimmune disease that causes pain, stiffness, swelling and limited motion and function of many joints. While RA can affect any joint, the small joints in the hands and feet tend to be involved most often. Inflammation sometimes can affect organs as well, for instance, the eyes or lungs. One possible mechanism of RA is that the immune system of patients is abnormal and attacks the body and creates inflammation. There is no cure for RA. Current treatments can lessen the symptoms and slow the dysfunction of the joints (Alten H. et al. 2008. Arthritis Research & Therapy. 10:R67.) Polynucleotide encoded antibodies of the present invention may reduce or eliminate symptoms in subjects inflicted with RA. Antibody levels achieved by therapeutic treatments of the present invention as well as outcomes of therapy may comprise any of the levels and outcomes as described by Alten et al (Alten H. et al. 2008. Arthritis Research & Therapy. 10:R67; the contents of which are herein incorporated by reference in their entirety.)

Cryopyrin is a protein thought to play a role in regulating inflammatory and apoptotic processes. Cryopyrin mutations and/or deficiencies are associated with a group of inflammatory diseases known as cryopyrin-associated periodic syndromes (CAPS.) These diseases include chronic infantile neurological cutaneous and articular (CINCA) syndrome, familial cold autoinflammatory syndrome (FCAS), neonatal-onset multisystem inflammatory disease (NOMID) and Muckle-Wells syndrome (MWS.) Symptoms are thought to be due, in large part, to over-expression of IL-1β (Lachmann, H. J. et al. 2009. J Exp Med. 206(5):1029-36.) In some embodiments, polynucleotide encoded antibodies of the present invention may neutralize all or a portion of IL-1β overexpressed in one or more forms of CAPS. Some treatments may be carried out on subjects ranging in age from very young to adult age. This includes subjects that may be about 4 years old and up.

Systemic juvenile idiopathic arthritis (SJIA) is an autoinflammatory disorder that may result in arthritis, fever, lymphadenopathy, rash, and serositis (Sikora, K. A. et al., 2011. Curr Opin Pediatr. 23(6):640-6.) Diagnosis may be difficult in many cases due to the transient nature of rashes and fever. In some cases, internal organs may affected. Polynucleotide encoded antibodies of the present invention may reduce or eliminate symptoms associated with SJIA. Some treatments may be carried out on subjects ranging in age from very young to adult age. This includes subjects that may be about two years old and up.

Chronic obstructive pulmonary disease (COPD) is an obstructive lung disease that comprises poor lung air flow. The disease is typically progressive and may result in a variety of symptoms that include cough, shortness of breath and elevated sputum production. Smoking is the most common cause of COPD, however other genetic and environmental factors may contribute to some cases (Wells, J. M. et al., 2013. Int J Chron Obstruct Pulmon Dis. 8:509-21.) Polynucleotide encoded antibodies of the present invention may reduce or eliminate symptoms associated with COPD.

In some cases, side effects associated with canakinumab treatment (including, but not limited to headache, vertigo, diarrhea, nausea, musculoskeletal pain, rhinitis, nasopharyngitis, bronchitis and increased susceptibility to influenza) may be avoided by replacement therapy with the polynucleotides of the invention.

Certain sequences encoding canakinumab fragments, domains or heavy or light chains are given in Table 9. The table is not an exhaustive list and any fragment or portion of the sequence may be encoded in the canakinumab polynucleotides of the invention.

TABLE 9
Table of Canakinumab Sequences

| | | SEQ |
|---|---|---|
| Description | Sequence | ID NO. |
| H-GAMMA-1 | QVQLVESGGGVVQPGRSLRLSCAASGFTFSVYGMN | 33 |
| (VH(1-118) + | WVRQAPGKGLEWVAIIWYDGDNQYYADSVKGRFT | |
| CH1(119-216) + | ISRDNSKNTLYLQMNGLRAEDTAVYYCARDLRTGP | |
| HINGE- | FDYWGQGTLVTVSSASTKGPSVFPLAPSSKSTSGGT | |
| REGION(217-231) + | AALGCLVKDYFPEPVTVSWNSGALTSGVHTFPAVL | |
| CH2(232-341) + | QSSGLYSLSSVVTVPSSSLGTQTYICNVNHKPSNTK | |
| CH3(342-448)) | VDKRVEPKSCDKTHTCPPCPAPELLGGPSVFLFPPKP | |
| | KDTLMISRTPEVTCVVVDVSHEDPEVKFNWYVDGV | |
| | EVHNAKTKPREEQYNSTYRVVSVLTVLHQDWLNG | |
| | KEYKCKVSNKALPAPIEKTISKAKGQPREPQVYTLP | |
| | PSREEMTKNQVSLTCLVKGFYPSDIAVEWESNGQPE | |
| | NNYKTTPPVLDSDGSFFLYSKLTVDKSRWQQGNVF | |
| | SCSVMHEALHNHYTQKSLSLSPGK | |
| L-KAPPA (V- | QVQLVESGGGVVQPGRSLRLSCAASGFTFSVYGMN | 34 |
| KAPPA(1-107) + | WVRQAPGKGLEWVAIIWYDGDNQYYADSVKGRFT | |
| C- | ISRDNSKNTLYLQMNGLRAEDTAVYYCARDLRTGP | |
| KAPPA(108-214)) | FDYWGQGTLVTVSSASTKGPSVFPLAPSSKSTSGGT | |
| | AALGCLVKDYFPEPVTVSWNSGALTSGVHTFPAVL | |
| | QSSGLEIVLTQSPDFQSVTPKEKVTITCRASQSIGSSL | |
| | HWYQQKPDQSPKLLIKYASQSFSGVPSRFSGSGSGT | |
| | DFTLTINSLEAEDAAAYYCHQSSSLPFTFGPGTKVDI | |
| | KRTVAAPSVFIFPPSDEQLKSGTASVVCLLNNFYPRE | |
| | AKVQWKVDNALQSGNSQESVTEQDSKDSTYSLSST | |
| | LTLSKADYEKHKVYACEVTHQGLSSPVTKSFNRGEC | |
| VH domain | QVQLVESGGGVVQPGRSLRLSCAASGFTFSVYGMN | 35 |
| | WVRQAPGKGLEWVAIIWYDGDNQYYADSVKGRFT | |
| | ISRDNSKNTLYLQMNGLRAEDTAVYYCARDLRTGP | |
| | FDYWGQGTLVTVSS | |
| VL domain | EIVLTQSPDFQSVTPKEKVTITCRASQSIGSSLHWYQ | 36 |
| | QKPDQSPKLLIKYASQSFSGVPSRFSGSGSGTDFTLT | |
| | INSLEAEDAAAYYCHQSSSLPFTFGPGTKVDIK | |

According to the present invention, the canakinumab polynucleotides of the present invention are engineered such that they may comprise one or more coding regions.

The coding regions of the canakinumab polynucleotides may encode any of the regions or portions of the canakinumab antibody. They may also further comprise coding regions not found in the original or parent canakinumab antibody.

The canakinumab polynucleotides of the present invention may be engineered to produce any of the 5 standard classes of immunoglobulins as shown in FIG. 1 using the canakinumab antibody or any of its component parts as a starting molecule.

The canakinumab polynucleotides may also be engineered according to the present invention to produce a variant canakinumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Cetuximab Parent Molecule or Antibody

According to the present invention, cetuximab polynucleotides or constructs and their associated cetuximab compositions are designed to produce the cetuximab antibody, a variant or a portion thereof in vivo.

Cetuximab is an antineoplastic agent also known as ERBITUX®.

Cetuximab, originally referred to as C225, is a chimeric antibody comprised of variable domain regions of a mouse anti-epidermal growth factor receptor (EGFr) monoclonal antibody, known as 225, and human IgG1 heavy and light chain constant domains. It interacts specifically with the N-terminal portion of the receptor and blocks binding of receptor ligand.

Chimerization of 225 was carried out to reduce murine portions of the antibody and decrease the likelihood of an anti-murine immune response in subjects receiving treatment. The resulting chimeric antibody, C225, was in fact more effective in reducing tumor growth using an established mouse model with a K_{D} that was about 5-fold lower than the mouse version of the antibody (Goldstein et al., 1995. Clin Canc Res. 1:1311-8, the contents of which are incorporated herein by reference in their entirety)

In some embodiments, polynucleotides of the present invention may encode antibodies comprising one or more components of cetuximab. Such antibodies may comprise variable domain regions from the murine antibody 225 as described in U.S. Pat. No. 6,217,866 and/or in Goldstein et al., 1995. Clin Canc Res. 1:1311-8; the contents of each of which are herein incorporated by reference in their entirety. Further, such antibodies may comprise chimeric combinations of such variable domain regions with human IgG1 components. Such combinations may comprise those found in antibody C225 as described by U.S. Pat. No. 6,217,866 and/or in Goldstein et al., 1995, Clin Canc Res. 1:1311-8, the contents of each of which are incorporated herein by reference in their entirety. Some polynucleotides described herein may encode one or more of the heavy and/or light chain anti-EGFr antibody sequences listed in Table 10. In some cases, polynucleotides of the present invention may encode one or more components of cetuximab (see cetuximab FDA label, incorporated herein by reference in its entirety)

Polynucleotides of the present invention may encode antibodies that specifically bind and block signaling activity of EGF receptors. Such antibodies may reduce and/or halt cell growth and/or proliferation of cells expressing or overexpressing such receptors. In some cases, this may reduce or eliminate cancerous cells and/or tumors.

EGFR is overexpressed in about a third of epithelial-derived cancers and enhanced signaling through this receptor is associated with tumor growth and proliferation (Mendelsohn, J. 2001. Endocr Relat Cancer. 8(1):3-9.) In some embodiments, polynucleotides of the present invention may be used as therapeutics to ultimately block EGFR activity and reduce or eliminate the spread of cancerous cells.

Colorectal cancer (CRC) comprises cancer of the colon, rectum or appendix and can lead to death in more than half of the patients suffering from the disease due to tumor metastasis (Xiang, B. 2013. Discov Med. 15(84):301-8.) A number of factors increase risk for the disease including gender, diet, smoking and level of physical activity. In some cases, polynucleotides of the present invention may be used to treat CRCs.

Administration of ERBITUX® causes severe infusion reactions in about 3% of patients that may be fatal in rare cases (see cetuximab FDA label.) In some cases, polynucleotide administration according to the present invention may offer the benefits of ERBITUX® treatment in such patients without the associated side effects.

Antibodies encoded by polynucleotides of the present invention may comprise a rate of clearance in subject of from about 0.2 L/h/m² to about 0.8 L/h/m². In some cases, such antibodies may comprise a volume of distribution of about 2 to about 3 L/m². The half life of such antibodies may comprise from about 75 hours to about 188 hours.

Certain sequences encoding cetuximab fragments, domains or heavy or light chains are given in Table 10. The table is not an exhaustive list and any fragment or portion of the sequence may be encoded in the cetuximab polynucleotides of the invention.

TABLE 10
Table of Cetuximab sequences

| | | SEQ |
|---|---|---|
| | | ID |
| Description | Sequence | NO |
| Anti-EGFR | QVQLKQSGPGLVQPSQSLSITCTVSGFSLTNYGVH | 37 |
| heavy | WVRQSPGKGLEWLGVIWSGGNTDYNTPFTSRLSI | |
| chain 1 | NKDNSKSQVFFKMNSLQSNDTAIYYCARALTYYD | |
| | YEFAYWGQGTLVTVSAASTKGPSVFPLAPSSKSTS | |
| | GGTAALGCLVKDYFPEPVTVSWNSGALTSGVHTF | |
| | PAVLQSSGLYSLSSVVTVPSSSLGTQTYICNVNHK | |
| | PSNTKVDKRVEPKSPKSCDKTHTCPPCPAPELLGG | |
| | PSVFLFPPKPKDTLMISRTPEVTCVVVDVSHEDPE | |
| | VKFNWYVDGVEVHNAKTKPREEQYNSTYRVVSV | |
| | LTVLHQDWLNGKEYKCKVSNKALPAPIEKTISKA | |
| | KGQPREPQVYTLPPSRDELTKNQVSLTCLVKGFYP | |
| | SDIAVEWESNGQPENNYKTTPPVLDSDGSFFLYSK | |
| | LTVDKSRWQQGNVFSCSVMHEALHNHYTQKSLS | |
| | LSPGK | |
| Anti-EGFr | DILLTQSPVILSVSPGERVSFSCRASQSIGTNIHWY | 38 |
| light | QQRTNGSPRLLIKYASESISGIPSRFSGSGSGTDFT | |
| chain 1 | LSINSVESEDIADYYCQQNNNWPTTFGAGTKLELK | |
| | RTVAAPSVFIFPPSDEQLKSGTASVVCLLNNFYPRE | |
| | AKVQWKVDNALQSGNSQESVTEQDSKDSTYSLSS | |
| | TLTLSKADYEKHKVYACEVTHQGLSSPVTKSFNR | |
| | GA | |

According to the present invention, the cetuximab polynucleotides of the present invention are engineered such that they may comprise one or more coding regions.

The coding regions of the cetuximab polynucleotides may encode any of the regions or portions of the cetuximab antibody. They may also further comprise coding regions not found in the original or parent cetuximab antibody.

The cetuximab polynucleotides of the present invention may be engineered to produce any of the 5 standard classes of immunoglobulins as shown in FIG. 1 using the cetuximab antibody or any of its component parts as a starting molecule.

The cetuximab polynucleotides may also be engineered according to the present invention to produce a variant cetuximab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

Certolizumab pegol Parent Molecule or Antibody

According to the present invention, certolizumab pegol polynucleotides or constructs and their associated certolizumab pegol compositions are designed to produce the certolizumab pegol antibody, a variant or a portion thereof in vivo.

Certolizumab pegol is an antirheumatic TNF blocker also known as CIMZIA® or CPD870.

Certolizumab pegol comprises an Fab antibody fragment that binds tumor necrosis factor (TNF)α. It is conjugated to a polyethylene glycol (PEG) moiety that is about 40 kDa in size. The PEG facilitates a delay in metabolism of the fragment leading to a longer half life in subjects being treated (Chapman A. P. et al., 1999. Nature Biotech. 17:780-3.)

Certolizumab pegol was derived humanizing a mouse anti-human TNFα antibody. The mouse antibody was selected based on high affinity for TNFα and the complementarity determining regions (CDRs) from that antibody were inserted into a human framework comprising an IgG Fab′ fragment. In addition to the CDRs, several other residues were also transferred to ensure structural integrity of the antigen-binding loop structures and maintenance of high affinity (Goel, N. et al., 2010. mAbs. 2(2):137-47.)

Polynucleotides of the present invention may encode an Fab′ fragment, such as any of those disclosed in U.S. Pat. Nos. 7,012,135, 7,186,820 and 7,402,662, the contents of each of which are herein incorporated by reference in their entirety. In some cases, polynucleotides may encode heavy and/or light chains or fragments thereof as disclosed in U.S. Pat. Nos. 7,012,135, 7,186,820 and 7,402,662 or as presented in Table 11.

In some cases, one or more antibodies produced by polynucleotides of the present invention may be conjugated to one or more PEG. Conjugation of such moieties may slow or prevent clearance of such antibodies from the circulation. PEG conjugation may comprise or be carried out according to any of the examples and/or methods described in Chapman A. P. et al., 1999. Nature Biotech. 17:780-3; the contents of which are herein incorporated by reference in their entirety.

Polynucleotides may encode one or more antibodies capable of binding TNFα and disrupting TNFα signaling activity. Such antibodies may be conjugated to one or more PEG moiety, such as any of those described by Chapment et al (Chapman A. P. et al., 1999. Nature Biotech. 17:780-3, the contents of which are herein incorporated by reference in their entirety.) PEG moieties may enable polynucleotide-encoded antibodies of the present invention to reduce, delay or avoid metabolic clearance

Polynucleotides of the present invention may be used according to any therapy where it is desired to reduce and/or eliminate the level of TNFα, whether it be systemic or localized. Such therapies may be desired in diseases and/or conditions that may include, but are not limited to septic shock, endotoxic shock, cardiovascular shock, inflammation, neurodegeneration, cancer, hepatitis, respiratory distress, arthritis, psoriasis, autoimmune diseases, Crohn's disease and transplanted tissue/organ rejection.

Rheumatoid arthritis is a chronic autoimmune disease that causes pain, stiffness, swelling and limited motion and function of many joints. While RA can affect any joint, the small joints in the hands and feet tend to be involved most often. Inflammation sometimes can affect organs as well, for instance, the eyes or lungs. One possible mechanism of RA is that the immune system of patients is abnormal and attacks the body and creates inflammation. There is no cure for RA. Current treatments can lessen the symptoms and slow the dysfunction of the joints (Alten H. et al. 2008. Arthritis Research & Therapy. 10:R67.) In some cases, polynucleotides of the present invention may be used to treat RA and alleviate or prevent one or more symptoms associated with that condition. Polynucleotide treatment may be carried out such that expression levels of translated products are about the same as the CDP870 antibody fragments used in Choy E. H. S. et al., 2002. Rheumatology. 41:1133-7, the contents of which are herein incorporated by reference in their entirety.

Psoriatic arthritis is a condition affecting as much as 30% of subjects suffering from psoriasis and is characterized by persistent inflammatory arthritis. The condition is progressive, leading to long-term tissue erosion and functional impairment in more than half of those afflicted (Mease, P. J. et al., 2013. Ann Rheum Dis. 73:48-55.) In some cases, polynucleotides of the present invention may be used to treat psoriatic arthritis and alleviate or prevent one or more symptoms associated with that condition.

Crohn's Disease (CD) is a debilitating disease that frequently causes diarrhea and abdominal cramps as well as fever, bleeding, and weight loss (Baran, B. et al., 2013. ISRN Gastroenterology. 2013:208073.) It is characterized by random regions of inflammation within any area of the gastrointestinal tract. In some cases, polynucleotides of the present invention may be used to treat Crohn's disease and alleviate or prevent one or more symptoms associated with that condition.

In cases where polynucleotides are used to treat psoriatic arthritis, primary endpoints may comprise American College of Rheumatology 20% (ACR20) response as determined at week 12 and/or modified Total Sharp Score change from baseline at the 24^{th} week of treatment (see Mease, P. J. et al., 2013. Ann Rheum Dis. 73:48-55 for details of these endpoints; the contents of which are herein incorporated by reference in their entirety.) Secondary endpoints may comprise Psoriatic Arthritis Response Criteria (PsARC) score, Psoriasis Area and Severity Index, Health Assessment Questionnaire Disability Index (HAQ-DI), Modified Nail Psoriasis Severity Index, Leeds Enthesitis Index and/or Leeds Dactylitis Index (Mease, P. J. et al., 2013. Ann Rheum Dis. 73:48-55.)

CIMZIA® treatment increases the risk of developing infections, with infections often developing in subjects that are also being treated with immunosuppressants that may include, but are not limited to methotrexate and/or corticosteroids (see CIMZIA® FDA label.) Such infections may include, but are not limited to tuberculosis (TB) infection and infections resulting from virus, fungi and/or bacterial proliferation. In some embodiments, treatment with polynucleotides of the present invention may be used to avoid the elevated infection risk associated with CIMZIA® treatment.

CIMZIA® treatment may increase the risk of developing one or more forms of cancer. Such cancers may include lymphoma. In some embodiments, treatment with polynucleotides of the present invention may be used to avoid the elevated risk of developing cancer associated with CIMZIA®.

Certain sequences encoding certolizumab pegol fragments, domains or heavy or light chains are given in Table 11. The table is not an exhaustive list and any fragment or portion of the sequence may be encoded in the certolizumab pegol polynucleotides of the invention.

TABLE 11
Table of Certolizumab Pegol Sequences

| | | SEQ |
|---|---|---|
| | | ID |
| Description | Sequence | NO. |
| Heavy chain | EVQLVESGGGLVQPGGSLRLSCAASGYVFTDYG | 39 |
| sequence | MNWVRQAPGKGLEWMGWINTYIGEPIYADSVK | |
| | GRFTFSLDTSKSTAYLQMNSLRAEDTAVYYCARG | |
| | YRSYAMDYWGQGTLVTVSSASTKGPSVFPLAPSS | |
| | KSTSGGTAALGCLVKDYFPEPVTVSWNSGALTSG | |
| | VHTFPAVLQSSGLYSLSSVVTVPSSSLGTQTYICN | |
| | VNHKPSNTKVDKKVEPKSCDKTHTCAA | |
| Light chain | DIQMTQSPSSLSASVGDRVTITCKASQNVGTNVA | 40 |
| sequence | WYQQKPGKAPKALIYSASFLYSGVPYRFSGSGSG | |
| | TDFTLTISSLQPEDFATYYCQQYNIYPLTFGQGTK | |
| | VEIKRTVAAPSVFIFPPSDEQLKSGTASVVCLLNN | |
| | FYPREAKVQWKVDNALQSGNSQESVTEQDSKDS | |
| | TYSLSSTLTLSKADYEKHKVYACEVTHQGLSSPV | |
| | TKSFNRGEC | |

According to the present invention, the certolizumab pegol polynucleotides of the present invention are engineered such that they may comprise one or more coding regions.

The coding regions of the certolizumab pegol polynucleotides may encode any of the regions or portions of the certolizumab pegol antibody. They may also further comprise coding regions not found in the original or parent certolizumab pegol antibody.

The certolizumab pegol polynucleotides of the present invention may be engineered to produce any of the 5 standard classes of immunoglobulins as shown in FIG. 1 using the certolizumab pegol antibody or any of its component parts as a starting molecule.

The certolizumab pegol polynucleotides may also be engineered according to the present invention to produce a variant certolizumab pegol antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Daclizumab Parent Molecule or Antibody

According to the present invention, daclizumab polynucleotides or constructs and their associated daclizumab compositions are designed to produce the daclizumab antibody, a variant or a portion thereof in vivo.

Daclizumab, also referred to as ZENAPAX, anti-Tac, anti-CD25, and anti-IL2Ralpha is an antibody developed by Protein Design Labs, Inc (3181 Porter Drive, Palo Alto, Calif. 94304) and subsequently acquired by Genentech and its parent company Roche.

Daclizumab is an immunosuppressant humanized IgG1 antibody used to prevent organ rejection in patients receiving a renal transplant. The monoclonal antibody binds to the IL2 receptor alpha subunit, also known as IL2Ralpha, CD25, or Tac.

Initial studies of immune suppression to counteract allograft rejection used agonist antibodies that suppressed all peripheral T-cells (Ortho Multicenter Transplant Study Group (1985) N. Engl. J. Med 313 (6):337-42). A murine anti-Tac antibody was shown to specifically target activated T-cells, eliminating complications due to a compromised immune response, but resulted in significant cross-species immunogenicity (Jaffers, G. J., Fuller, T. C., Cosimi, A. B., Russell, P. S., Winn, H. J. & Colvin, R. B. (1986) Transplantation 41, 572-578). A chimeric humanized anti-Tac antibody was developed with the murine CDR sequences to reduce immunogenicity complications (C Queen, W P Schneider, H E Selick, P W Payne, N F Landolfi, J F Duncan, N M Avdalovic, M Levitt, R P Junghans, and T A Waldmann Proc Natl Acad Sci USA. 1989 December; 86(24): 10029-10033).

Daclizumab is a composite of human (90%) and murine (10%) antibody sequences. The human sequences were derived from the constant domains of human IgG1 and the variable framework regions of the Eu myeloma antibody. The murine sequences were derived from the CDRs of a murine anti-Tac antibody.

In some embodiments, polynucleotide encoded antibodies according to the present invention may comprise one or more portions of the antibody as described by WO Patent No. 8909622 and U.S. Pat. Nos. 5,693,761 and 7,521,054, the contents of each of which are herein incorporated by reference in their entirety. Such antibodies may comprise one or more portions of the variable regions, complementarity determining regions (CDR), and/or antigen binding region of antibodies as described by WO Patent No. 8909622 and U.S. Pat. Nos. 5,693,761 and 7,521,054, the contents of each of which are herein incorporated by reference in their entirety.

Daclizumab functions as an IL-2 receptor antagonist that inhibits IL-2 binding to the IL-2 receptor complex. Daclizumab binding is highly specific for Tac, which is expressed on activated but not resting lymphocytes. Administration of Daclizumab inhibits IL-2-mediated activation of lymphocytes, a critical pathway in the cellular immune response involved in allograft rejection.

The daclizumab polynucleotides of the present invention may be used as part of an immunosuppressive regimen including cyclosporine, mycophenolate mofetil, and corticosteroids. Prophylaxis of acute rejection has been demonstrated in recipients of kidney allografts when treated with daclizumab. Prophylaxis of acute rejection of other solid organs has not been demonstrated.

Several side effects and contraindications have been identified for daclizumab including but not limited to: gastrointestinal system (constipation, nausea, diarrhea, vomiting, abdominal pain, pyrosis, dyspepsia, abdominal distention, and epigastric pain not food-related); metabolic system (edema extremities, edema); central and peripheral nervous system (tremor, headache, dizziness); urinary system (oliguria, dysuria, renal tubular necrosis); general (posttraumatic pain, chest pain, fever, pain, fatigue); autonomic nervous system (hypertension, hypotension, aggravated hypertension); respiratory system (dyspnea, pulmonary edema, coughing); skin and appendages (impaired wound healing without infection, acne); psychiatric (insomnia); musculoskeletal system: (musculoskeletal pain, back pain); heart rate and rhythm (tachycardia); vascular extracardiac (thrombosis); platelet, bleeding and clotting disorders (bleeding); hemic and lymphatic (lymphocele); gastrointestinal system (flatulence, gastritis, hemorrhoids); metabolic and nutritional (fluid overload, diabetes mellitus, dehydration); urinary system (renal damage, hydronephrosis, urinary tract bleeding, urinary tract disorder, renal insufficiency); general (shivering, generalized weakness); central and peripheral nervous system (urinary retention, leg cramps, prickly sensation); respiratory system (atelectasis, congestion, pharyngitis, rhinitis, hypoxia, rales, abnormal breath sounds, pleural effusion); skin and appendages (pruritus, hirsutism, rash, night sweats, increased sweating); psychiatric (depression, anxiety); musculoskeletal system (arthralgia, myalgia); vision (vision blurred); application site (application site reaction). It is expected that the polynucleotides of the present invention would avoid some or all of such side effects.

Certain sequences encoding daclizumab fragments, domains or heavy or light chains are given in Table 12. The table is not an exhaustive list and any fragment or portion of the sequence may be encoded in the daclizumab polynucleotides of the invention. In the table, the CDRs are underlined in the light and heavy chains.

TABLE 12
Table of Daclizumab Sequences

| | | SEQ ID |
|---|---|---|
| Description | Sequence | NO. |
| Heavy Chain | QVQLQQSGAELAKPGASVKMSCKASGYTF | 41 |
| | TSYRMHWVKQRPGQGLEWIGYINPSTGYT | |
| | EYNQKFKDKATLTADKSSSTAYMQLSSLTF | |
| | EDSAVYYCARGGGVFDYWGQGTTLTVSS | |
| Light Chain | QIVLTQSPAIMSASPGEKVTITCSASSSISYM | 42 |
| | HWFQQKPGTSPKLWIYTTSNLASGVPARFS | |
| | GSGSGTSYSLTISRMEAEDAATYYCHQRST | |
| | YPLTFGSGTKLELK | |
| Heavy Chain | SYRMH | 43 |
| CDR1 | | |
| Heavy Chain | YINPSTGYTEYNQKFKD | 44 |
| CDR2 | | |
| Heavy Chain | GGGVFDY | 45 |
| CDR3 | | |
| Light Chain | SASSSISYMH | 46 |
| CDR1 | | |
| Light Chain | TTSNLAS | 47 |
| CDR2 | | |
| Light Chain | HQRSTYPLT | 48 |
| CDR3 | | |

According to the present invention, the daclizumab polynucleotides of the present invention are engineered such that they may comprise one or more coding regions.

The coding regions of the daclizumab polynucleotides may encode any of the regions or portions of the daclizumab antibody. They may also further comprise coding regions not found in the original or parent daclizumab antibody.

The daclizumab polynucleotides of the present invention may be engineered to produce any of the 5 standard classes of immunoglobulins as shown in FIG. 1 using the daclizumab antibody or any of its component parts as a starting molecule.

The daclizumab polynucleotides may also be engineered according to the present invention to produce a variant daclizumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Denosumab Parent Molecule or Antibody

According to the present invention, denosumab polynucleotides or constructs and their associated denosumab compositions are designed to produce the denosumab antibody, a variant or a portion thereof in vivo.

Denosumab is also known as AMG 162, Prolia, Ranmark, and/or Xgeva.

The Denosumab commercial antibody was developed by Amgen, Inc. and is currently marketed under two trade names; Prolia for the treatment of post-menopausal osteoporosis and Xgena for the treatment of bone metastases from solid tumors.

Denosumab is a fully human IgG2kappa monoclonal antibody that is used to prevent bone loss in osteopenic disorders including osteoporosis and solid tumor bone metastases. Bone remodeling is a homeostatic process that is balanced by the activity of osteoblasts (bone formation) and osteoclasts (bone degradation or resorption). The coupling of this system is achieved by three factors: receptor activator of nuclear factor kappa-B (RANK), receptor activator of nuclear factor kappa-B ligand (RANKL) and osteoprotegrin (OPG). RANKL binding to RANK promotes osteoclast differentiation, leading to increased bone resorption. OPG binding to RANKL, blocks RANKL binding to RANK and therefore decreases bone resorption (Westenfeld R et al., 2006 Nephrol Dial Transplant. 21 (8):2075-7). The inhibition of osteoclast differentiation, without directly altering osteoblast activity, has the net effect of increasing bone formation.

A study examining bone loss in postmenopausal women demonstrated decreased bone remodeling following a single dose of osteoprotegrin (OPG), an inhibitor of receptor activator of nuclear factor kappa-B (RANKL) (Bekker P J et al., 2001. J Bone Miner Res. 16(2):348-60).

Denosumab, originally referred to as AMG 162, was designed as a specific agonist inhibitor of RANKL activity. A Phase I safety trial conducted with postmenopausal women demonstrated that one subcutaneous injection of Denomusab resulted in a dose dependent, sustained decrease in bone loss (Bekker P J et al., 2004. J Bone Miner Res. 19(7):1059-66). Multiple clinical trials are investigating the effectiveness of denosumab in the treatment of multiple myeloma and bone metastases associated with various cancers including, but not limited to, breast cancer (Body J J et al., 2006. Clin Cancer Res. 15; 12(4):1221-8).

In some embodiments, polynucleotide encoded antibodies according to the present invention may comprise one or more portions of the antibody as described by U.S. Pat. Nos. 7,364,736, 8,058,418 and 8,409,578, the contents of each of which are herein incorporated by reference in their entirety. Such nucleotide-derived antibodies may comprise one or more portions of the variable regions, complementarity determining regions (CDR), and/or antigen binding region of antibodies as described by U.S. Pat. Nos. 7,364,736, 8,058,418 and 8,409,578, the contents of each of which are herein incorporated by reference in their entirety.

Polynucleotide encoded antibodies of the present invention may directly bind to RANKL, preventing RANKL binding to RANK. RANKL targeting by nucleotide-derived antibodies of the present invention may inhibit osteoclast formation, function, and survival. Inhibition of osteoclast formation, function, and survival may alter bone remodeling dynamics such that bone resorption is decreased and bone formation is increased.

The polynucleotide encoded antibodies of the present invention may be used to prevent bone resorption that leads to bone brittleness and fractures. Osteopenic disorders and certain cancers discussed herein are known to increase osteoclast activity and induce bone resorption. Breast, prostate, and multiple myeloma cancers are now known to produce factors that result in the over-expression of RANKL in the bone, and lead to increased osteoclast numbers and activity. Postmenopausal osteoporosis in women is a disease state of increased bone resorption resulting in brittle bones that may occur in older populations due to changes in the dynamics of bone remodeling factors. Osteopenic disorders may occur in conjunction with other factors and/or diseases including but not limited to adjuvant aromatase inhibitor therapy during breast cancer treatment, and men receiving androgen deprivation therapy for non-metastatic prostate cancer.

Contraindications associated with denosumab include severe or fatal hypocalcemia; hypersensitivity reactions including hypotension, dyspnea, upper airway edema, lip swelling, rash, pruritis, urticarial, and anaphylaxis; osteonecrosis of the jaw, atypical femoral fractures, and embryo-fetal toxicity.

The polynucleotide encoded antibodies of the present invention may advantageously reduce or eliminate such contraindications.

As used herein, the term “osteopenic disorder” refers to conditions that directly or indirectly lead to bone loss and/or brittleness and/or fracture including, but not limited to: breast, prostate, and multiple myeloma; osteoporosis, osteopenia, Paget's disease, lytic bone metastases, periodontitis, rheumatoid arthritis, and bone loss due to immobilization.

Certain sequences encoding denosumab fragments, domains or heavy or light chains are given in Table 13. The table is not an exhaustive list and any fragment or portion of the sequence may be encoded in the denosumab polynucleotides of the invention.

TABLE 13
Table of Denosumab Sequences

| | | SEQ |
|---|---|---|
| | | ID |
| Description | Sequence | NO. |
| Heavy Chain | MEFGLSWLFLVAILKGVQCEVQLLESGGGLVQPGG | 49 |
| | SLRLSCAASGFTFSSYAMSWVRQAPGKGLEWVSGI | |
| | TGSGGSTYYADSVKGRFTISRDNSKNTLYLQMNSL | |
| | RAEDTAVYYCAKDPGTTVIMSWFDPWGQGTLVTV | |
| | SSASTKGPSVFPLAPCSRSTSESTAALGCLVKDYFP | |
| | EPVTVSWNSGALTSGVHTFPAVLQSSGLYSLSSVV | |
| | TVPSSNFGTQTYTCNVDHKPSNTKVDKTVERKCCV | |
| | ECPPCPAPPVAGPSVFLFPPKPKDTLMISRTPEVTC | |
| | VVVDVSHEDPEVQFNWYVDGVEVHNAKTKPREEQF | |
| | NSTFRVVSVLTVVHQDWLNGKEYKCKVSNKGLPAPI | |
| | EKTISKTKGQPREPQVYTLPPSREEMTKNQVSLTCL | |
| | VKGFYPSDIAVEWESNGQPENNYKTTPPMLDSDGS | |
| | FFLYSKLTVDKSRWQQGNVFSCSVMHEALHNHYT | |
| | QKSLSLSPGK | |
| Light Chain | METPAQLLFLLLLWLPDTTGEIVLTQSPGTLSLSPG | 50 |
| | ERATLSCRASQSVRGRYLAWYQQKPGQAPRLLIYG | |
| | ASSRATGIPDRFSGSGSGTDFTLTISRLEPEDFAVF | |
| | YCQQYGSSPRTFGQGTKVEIKRTVAAPSVFIFPPSD | |
| | EQLKSGTASVVCLLNNFYPREAKVQWKVDNALQSG | |
| | NSQESVTEQDSKDSTYSLSSTLTLSKADYEKHKVY | |
| | ACEVTHQGLSSPVTKSFNRGEC | |
| Heavy Chain | VQLLESGGGLVQPGGSLRLSCAASGFTFSSYAMSW | 51 |
| Variable | VRQAPGKGLEWVSGITGSGGSTYYADSVKGRFTIS | |
| Region | RDNSKNTLYLQMNSLRAEDTAVYYCAKDPGTTVI | |
| | MSWFDPWGQGTLVTVSS | |
| Light Chain | EIVLTQSPGTLSLSPGERATLSCRASQSVRGRYLAW | 52 |
| Variable | YQQKPGQAPRLLIYGASSRATGIPDRFSGSGSGTDF | |
| Region | TLTISRLEPEDFAVFYCQQYGSSPRTFGQGTKVEIK | |

According to the present invention, the denosumab polynucleotides of the present invention are engineered such that they may comprise one or more coding regions.

The coding regions of the denosumab polynucleotides may encode any of the regions or portions of the denosumab antibody. They may also further comprise coding regions not found in the original or parent denosumab antibody.

The denosumab polynucleotides of the present invention may be engineered to produce any of the 5 standard classes of immunoglobulins as shown in FIG. 1 using the denosumab antibody or any of its component parts as a starting molecule.

The denosumab polynucleotides may also be engineered according to the present invention to produce a variant denosumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Eculizumab Parent Molecule or Antibody

According to the present invention, eculizumab polynucleotides or constructs and their associated eculizumab compositions are designed to produce the eculizumab antibody, a variant or a portion thereof in vivo.

Eculizumab also known as 5G1.1 or the trade name Soliris is a commercial antibody packaged by Alexion Parmaceuticals Inc. and Ben Venue Laboratories Inc. under the trade name Soliris for the treatment of paroxysmal nocturnal hemoglobinuria (PNH).

Eculizumab is a recombinant humanized monoclonal IgG2/4;κ antibody produced from Hybridoma 5G1.1 having ATCC designation HB-11625. Eculizumab contains human constant regions from human IgG2 sequences and human IgG4 sequences and murine complementarity-determining regions (CDRs) grafted onto the human framework light- and heavy-chain variable regions.

A genetic mutation in PNH patients leads to the generation of populations of abnormal red blood cells (RBCs) that are deficient in terminal complement inhibitors (CD-59), rendering PNH RBCs sensitive to persistent terminal complement-mediated destruction. The destruction and loss of these PNH cells (intravascular hemolysis) results in low RBC counts (anemia) and also fatigue, difficulty in functioning, pain, dark urine, shortness of breath, and blood clots.

Eculizumab, is a monoclonal antibody that binds to the complement protein C5 specifically and with high affinity, thereby inhibiting its cleavage to C5a and C5b and subsequent generation of the terminal complement complex C5b-9. Eculizumab inhibits terminal complement mediated intravascular hemolysis in PNH patients and therefore the destruction of PNH erythrocytes that lack complement protection with CD-59.

A study examining patients with paroxysmal nocturnal hemoglobulinuria demonstrated decreased intravascular hemolysis and requirement for transfusion (Hillmen P et al. 2004 N Engl J Med. 5; 350(6):552-9).

In some embodiments, polynucleotide encoded antibodies according to the present invention may comprise one or more portions of the antibody as described by U.S. Pat. No. 6,074,642, WO1995029697, and U.S. Pat. No. 6,355,245, the contents of each of which are herein incorporated by reference in their entirety. Such nucleotide-derived antibodies may comprise one or more portions of the variable regions, complementarity determining regions (CDR), and/or antigen binding region of antibodies as described by U.S. Pat. No. 6,074,642, WO1995029697, and U.S. Pat. No. 6,355,245, the contents of each of which are herein incorporated by reference in their entirety.

The polynucleotide encoded antibodies of the present invention may directly bind to complement C5, preventing proteolytic degradation of RBCs that are deficient in terminal complement inhibitor CD-59.

The polynucleotide encoded antibodies of the present invention may be used to reduce hemolysis in individuals with paroxysmal nocturnal hemoglobinuria (PNH). Nucleotide-derived antibodies of the present invention may also be used to inhibit complement-mediated thrombotic microangiopathy in individuals with atypical hemolytic uremic syndrome (aHUS).

Contraindications associated with eculizumab antibodies include unresolved serious Neisseria meningitides infection and patients who are not currently vaccinated against Neisseria meningitides. Other symptoms associated with eculizumab include, but are not limited to headache, nasopharyngitis, back pain, nausea, hypertension, upper respiratory tract infection, diarrhea, anemia, vomiting, urinary tract infection, and leukopenia. It is expected that the polynucleotide encoded antibodies of the present invention may overcome some or all of the contraindications or side effects associated with the eculizumab commercial antibody.

Certain sequences encoding eculizumab fragments, domains or heavy or light chains are given in Table 14. The table is not an exhaustive list and any fragment or portion of the sequence may be encoded in the eculizumab polynucleotides of the invention.

TABLE 14
Table of Eculizumab Sequences

| | | SEQ |
|---|---|---|
| | | ID |
| Description | Sequence | NO. |
| Heavy Chain | QVQLQQSGAELMKPGASVKMSCKATGYIFSN | 53 |
| | YWIQWIKQRPGHGLEWIGEILPGSGSTEYTEN | |
| | FKDKAAFTADTSSNTAYMQLSSLTSEDSAVY | |
| | YCARYFFGSSPNWYFDVWGAGTTVTVSS | |
| Light Chain | DIQMTQSPASLSASVGETVTITCGASENIYGA | 54 |
| | LNWYQRKQGKSPQLLIYGATNLADGMSSRFSG | |
| | SGSGRQYYLKISSLHPDDVATYYCQNVLNTPL | |
| | TFGAGTKLELK | |
| Heavy Chain | NYWIQ | 55 |
| CDR1 | | |
| Heavy Chain | EILPGSGSTEYTENFKD | 56 |
| CDR2 | | |
| Heavy Chain | YFFGSSPNWYFDV | 57 |
| CDR3 | | |
| Light Chain | GASENIYGALN | 58 |
| CDR1 | | |
| Light Chain | GATNLAD | 59 |
| CDR2 | | |
| Light Chain | VLNTPLT | 60 |
| CDR3 | | |
| Heavy Chain | QVQLVQSGAEVKKPGASVKVSCKASGYIFSN | 61 |
| (Genetic | YWIQWVRQAPGQGLEWMGEILPGSGSTEYTE | |
| Recombination) | NFKDRVTMTRDTSTSTVYMELSSLRSEDTAV | |
| | YYCARYFFGSSPNWYFDVWGQGTLVTVSSAST | |
| | KGPSVFPLAPCSRSTSESTAALGCLVKDYFPE | |
| | PVTVSWNSGALTSGVHTFPAVLQSSGLYSLSS | |
| | VVTVPSSNFGTQTYTCNVDHKPSNTKVDKTV | |
| | ERKCCVECPPCPAPPVAGPSVFLFPPKPKDTL | |
| | MISRTPEVTCVVVDVSQEDPEVQFNWYVDGV | |
| | EVHNAKTKPREEQFNSTYRVVSVLTVLHQDW | |
| | LNGKEYKCKVSNKGLPSSIEKTISKAKGQPRE | |
| | PQVYTLPPSQEEMTKNQVSLTCLVKGFYPSDI | |
| | AVEWESNGQPENNYKTTPPVLDSDGSFFLYSR | |
| | LTVDKSRWQEGNVFSCSVMHEALHNHYTQK | |
| | SLSLSLGK | |
| Light Chain | DIQMTQSPSSLSASVGDRVTITCGASENIYGA | 62 |
| (Genetic | LNWYQQKPGKAPKLLIYGATNLADGVPSRFSG | |
| Recombination) | SGSGTDFTLTISSLQPEDFATYYCQNVLNTPL | |
| | TFGQGTKVEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |

According to the present invention, the eculizumab polynucleotides of the present invention are engineered such that they may comprise one or more coding regions.

The coding regions of the eculizumab polynucleotides may encode any of the regions or portions of the eculizumab antibody. They may also further comprise coding regions not found in the original or parent eculizumab antibody.

The eculizumab polynucleotides of the present invention may be engineered to produce any of the 5 standard classes of immunoglobulins as shown in FIG. 1 using the eculizumab antibody or any of its component parts as a starting molecule.

The eculizumab polynucleotides may also be engineered according to the present invention to produce a variant eculizumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Golimumab Parent Molecule or Antibody

According to the present invention, golimumab polynucleotides or constructs and their associated golimumab compositions are designed to produce the golimumab antibody, a variant or a portion thereof in vivo.

Golimumab, also known as SIMPONI®, CNTO148 and TNV148B, is a human IgG1-kappa monoclonal antibody derived from immunizing genetically engineered mice with human TNF-alpha developed by Centocor and Janessen Biotech in collaboration with Schering-Plough and Mitsubishi Tanabe Pharma. Golimumab is similar to the antibody Infliximab, except that is has been engineering to be fully human and it is usually given as a subcutaneous injection. Golimumab binds and inhibits soluble and transmembrane human TNF-alpha which is beneficial in those suffering from chronic inflammation caused by an increase in TNF-alpha. Golimumab was approved in 2009 by the FDA for the treatment of moderately-to-severely active rheumatoid arthritis (RA), active psoriatic arthritis (PsA), and active ankylosing spondylitis (AS) in adults. Golimumab may be used as an adjunct to methotrexate treatment in subjects with RA or PsA. In 2013, Golimumab was approved by the FDA to treat adults with moderate to severe ulcerative colitis.

However, some subjects have suffered from serious infections leading to hospitalization or death as a result of receiving Golimumab treatments. Therefore, there is a need in the art to develop a safer alternative to delivering the Golimumab antibody to a subject in order to treat the subject in need thereof.

In one embodiment, the polynucleotides described herein encode a human IgG1-kappa golimumab monoclonal antibody or a fragment or variant thereof. These polynucleotide encoded antibodies can bind to and inhibit soluble and transmembrane human TNF-alpha. The inhibition of TNF-alpha can prevent the binding of TNF-alpha to its receptors which can prevent both leukocyte infiltration through prevention of cell adhesion proteins such as, but not limited to, E-selectin, ICAM-1 and VCAM-1, and pro-inflammatory cytokine secretion such as, but not limited to, IL-6, IL-8, G-CSF and GM-CSF. As a non-limiting example, the polynucleotide can encode Golimumab polypeptides or a fragment or variant thereof.

In one embodiment, the polynucleotides described herein encode an antibody that does not bind to or neutralize other TNF superfamily ligands such as, but not limited to, lymphotoxin. As a non-limiting example, the polynucleotide can encode Golimumab polypeptides or a fragment or variant thereof.

In one embodiment, the polynucleotides described herein encode a modulator of a target, such as, but not limited to, tumor necrosis factor (TNF). As a non-limiting example, the polynucleotide can encode Golimumab polypeptides or a fragment or variant thereof which is known to modulate TNF-alpha in a subject.

In one embodiment, the polynucleotides described herein encode a tumor necrosis factor (TNF) blocker. The polynucleotides may then be used to reduce the effects of a substance in the body that can cause inflammation in a subject. As a non-limiting example, the polynucleotide can encode Golimumab polypeptides or a fragment or variant thereof.

In one embodiment, prior to treatment with the polynucleotides described herein a subject may be tested for infections such as, but not limited to, tuberculosis (TB).

In one embodiment, prior to and/or during treatment with polynucleotides described herein Hepatitis B infection is monitored with those undergoing treatment.

In one embodiment, the polynucleotides described herein may encode Golimumab or a fragment or variant thereof and may be used to treat a neurological disorder such as, but not limited to, certain forms of dementia.

In one embodiment, the polynucleotides described herein may encode Golimumab or a fragment or variant thereof and may be used to treat asthma.

In one embodiment, the polynucleotides described herein may encode may eliminate some, if not all, of the side effects associated with the commercial golimumab antibody. Such side effects include, but not limited to, body aches or pain, chills, cough, difficulty with breathing, ear congestion, fever, headache, loss of voice, muscle aches, sneezing, sore throat, stuffy or runny nose, unusual tiredness or weakness, blurred vision, feeling such as burning, crawling, itching, numbness, prickling, “pins and needles”, or tingling, congestion, cough with mucus, diarrhea, dizziness, general feeling of discomfort or illness, hoarseness, joint pain, loss of appetite, muscle aches and pains, nausea, nervousness, pain or tenderness around the eyes and cheekbones, painful cold sores or blisters on the lips, pounding in the ears, shivering, shortness of breath or troubled breathing, slow or fast heartbeat, sweating, tender, swollen glands in the neck, tightness of the chest or wheezing, trouble with sleeping, trouble with swallowing, voice changes, vomiting, bone pain, frequent or painful urination, pain and inflammation at the joints, redness, soreness, or itching skin, severe abdominal or stomach pain, sores, welting, or blisters, and/or yellow eyes or skin.

In one embodiment, the polynucleotides described herein encoding golimumab polypeptide or a fragment or variant thereof are formulated for subcutaneous administration. As a non-limiting example, the formulation may be a subcutaneous injection solution with 100 mg of drug in 1 mL of solution or 50 mg of drug in 0.5 mL of solution. The formulation may be stored for administration in a prefilled syringe such as, but not limited to a SMARTJECT® autoinjector.

In one embodiment, the polynucleotides described herein are formulated for perispinal extrathecal injection. (See e.g., the administration methods of Golimumab by perispinal administration as described in U.S. Pat. No. 7,629,311 or US Patent Application No US20130022540, the contents of which each of which are herein incorporated by reference in their entirety).

In one embodiment, the golimumab antibodies encoded by the polynucleotides of the invention have a half-life of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 day or at least 14 days.

In one embodiment, the dose of the polynucleotides may be between 10 and 100 mg, including, but not limited to, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg and 100 mg.

In one embodiment, the polynucleotides described may be formulated for monthly subcutaneous injection for the treatment of ankylosing spondylitis or psoriatic arthritis. As a non-limiting example, the amount of drug administered may be 50 mg per 0.5 mL. The treatment may be administered with or without methotrexate or other non-biologic Disease Modifying Antirheumatic Drugs (DMARDs).

In one embodiment, the polynucleotides described herein may be formulated for subcutaneous injections at Week 0, Week 2, Week 4 and every 4 weeks following Week 4 for the treatment of Ulcerative Colitis. As a non-limiting example, the amount of drug administered may be 200 mg of drug at week 0, 100 mg of drug at week 2, 100 mg at Week 4 and 100 mg every 4 weeks after Week 4.

In one embodiment, the polynucleotides described herein are formulated for infusion administration. As a non-limiting example, the infusion administration may be used in the treatment of moderately to severely active rheumatoid arthritis.

In another embodiment, the polynucleotides described herein may be formulated for intravenous infusion for the treatment of rheumatoid arthritis at Week 0, Week 4 and every 8 weeks after Week 4. As a non-limiting example, the amount of drug administered may be 2 mg per kg administered over 30 minutes. The treatment may be administered with or without methotrexate. The polynucleotides may be administered while a subject is taking, corticosteroids, non-biologic Disease Modifying Antirheumatic Drugs (DMARDs), and/or non-steroidal anti-inflammatory drugs (NSAIDs).

Certain sequences encoding golimumab fragments, domains or heavy or light chains are given in Table 15. The table is not an exhaustive list and any fragment or portion of the sequence may be encoded in the golimumab polynucleotides of the invention.

TABLE 15
Table of Golimumab Sequences

| | | SEQ ID |
|---|---|---|
| Description | Sequence | NO |
| Heavy Chain Variable Region (See | QVQLVESGGGVVQPGRSLRLS | 63 |
| U.S. Pat. No. 7,250,165, SEQ ID | CAASGFIFSSYAMHWVRQAPG | |
| NO: 7 and Int'l Patent Pub | NGLEWVAFMSYDGSNKKYAD | |
| WO2013087912, FIG. 2F and SEQ | SVKGRFTISRDNSKNTLYLQMN | |
| ID NO: 6; the contents of each of | SLRAEDTAVYYCARDRGIAAG | |
| which are herein incorporated by | GNYYYYGMDVWGQGTTVTVSS | |
| reference in their entirety) | | |
| Light Chain Variable Region (See U.S. | EIVLTQSPATLSLSPGERATLSC | 64 |
| Pat. No. 7,250,165, SEQ ID NO: 8 | RASQSVYSYLAWYQQKPGQAP | |
| and Int'l Patent Pub WO2013087912, | RLLIYDASNRATGIPARFSGSGS | |
| FIG. 2G and SEQ ID NO: 7; the | GTDFTLTISSLEPEDFAVYYCQ | |
| contents of each of which are herein | QRSNWPPFTFGPGTKVDIK | |
| incorporated by reference in their | | |
| entirety) | | |
| Heavy Chain CDR1 (See Int'l Patent | GFIFSSYAMH | 65 |
| Pub WO2013087912, FIG. 2F and | | |
| SEQ ID NO: 6; the contents of which | | |
| are herein incorporated by reference | | |
| in its entirety) | | |
| Heavy Chain CDR2 (See Int'l Patent | FMSYDGSNKKYADSVKG | 66 |
| Pub WO2013087912, FIG. 2F and | | |
| SEQ ID NO: 6; the contents of which | | |
| are herein incorporated by reference | | |
| in its entirety) | | |
| Heavy Chain CDR3 (See Int'l Patent | DRGIAAGGNYYYYGMDV | 67 |
| Pub WO2013087912, FIG. 2F and | | |
| SEQ ID NO: 6; the contents of which | | |
| are herein incorporated by reference | | |
| in its entirety) | | |
| Light Chain CDR1 (See Int'l Patent | RASQSVYSYLA | 68 |
| Pub WO2013087912, FIG. 2G and | | |
| SEQ ID NO: 7; the contents of which | | |
| are herein incorporated by reference | | |
| in its entirety) | | |
| Light Chain CDR2 (See Int'l Patent | DASNRAT | 69 |
| Pub WO2013087912, FIG. 2G and | | |
| SEQ ID NO: 7; the contents of which | | |
| are herein incorporated by reference | | |
| in its entirety) | | |
| Light Chain CDR3 (See Int'l Patent | QQRSNWPPFT | 70 |
| Pub WO2013087912, FIG. 2G and | | |
| SEQ ID NO: 7; the contents of which | | |
| are herein incorporated by reference | | |
| in its entirety) | | |

According to the present invention, the golimumab polynucleotides of the present invention are engineered such that they may comprise one or more coding regions.

The coding regions of the golimumab polynucleotides may encode any of the regions or portions of the golimumab antibody. They may also further comprise coding regions not found in the original or parent golimumab antibody.

The golimumab polynucleotides of the present invention may be engineered to produce any of the 5 standard classes of immunoglobulins as shown in FIG. 1 using the golimumab antibody or any of its component parts as a starting molecule.

The golimumab polynucleotides may also be engineered according to the present invention to produce a variant golimumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Ibritumomab Parent Molecule or Antibody

According to the present invention, ibritumomab polynucleotides or constructs and their associated ibritumomab compositions are designed to produce the ibritumomab antibody, a variant or a portion thereof in vivo.

Ibritumomab also known as ZEVALIN® is a commercial monoclonal antibody that binds CD20 found on B cell surfaces. It is typically conjugated with the chelator tiuxetan, which facilitates attachment of a radioisotope such as yttrium-90 or indium-111. It is often administered in coordination with Rituximab which also targets CD20. The two antibodies share similar VH domains. In some cases, administration is used to treat refractory non-Hodgkin's lymphoma (NHL) that develops after primary cancer treatment (Wagner, H. N. et al., 2002. J Nucl Med. 43(2):267-72.)

Ibritumomab comprises heavy and light chains of mouse origin, coming from the monoclonal antibody IDEC-Y2B8 (WHO Drug Information 14(1), 2000. List 43.) When conjugated with a radioisotope, ibritumomab is used to kill both normal and malignant B cells that express CD20, while sparing B cell precursors to allow for repopulation with healthy B cells (Hainsworth, J. D. 2000. Oncologist. 5(5):376-84.)

Polynucleotides of the present invention may encode one or more antibodies capable of binding CD20. In some cases, such antibodies may comprise the amino acid sequence of all or a portion of the commercial antibody, ibritumomab. Polynucleotides may encode heavy and/or light chain amino acid sequences of monoclonal antibody Y2B8 as described in U.S. Pat. Nos. 5,736,137 and 5,776,456, the contents of each of which are herein incorporated by reference in their entirety. Some polynucleotides may encode one or more of the amino acid sequences listed in Table 16. Further polynucleotides of the invention may encode one or more components of any of the antibodies described in U.S. Pat. Nos. 5,736,137, 5,776,456, 6,399,061, 7,682,612, 7,744,877 and/or 8,557,244, the contents of each of which are herein incorporated by reference in their entirety.

Antibodies produced by one or more polynucleotides disclosed herein may be conjugated with one or more chelator. Such chelators may comprise tiuxetan. In some cases chelated antibodies may be combined with one or more radioisotope. Such radioisotopes may include, but are not limited to yttrium-90 or indium-111.

Administration of polynucleotide encoded antibodies of the present invention that have been conjugated with one or more radioisotope may be carried out according to any of the guidance provided on the FDA label for ibritumomab tiuxetan, the contents of which are herein incorporated by reference in their entirety.

In some cases, treatment with such polynucleotides of the invention may be carried out in order to reduce or eliminate mature B lymphocyte populations. In some cases, such treatments may be used to treat one or more forms of cancer. Such cancers may include, but are not limited to non-Hodgkin's lymphoma (NHL.)

Non-Hodgkin's lymphoma (NHL) refers to any of a large group of cancers of lymphocytes (white blood cells). Non-Hodgkin lymphomas can occur at any age and are often marked by lymph nodes that are larger than normal, fever, and weight loss. There are many different types of non-Hodgkin lymphoma. These types can be divided into aggressive (fast-growing) and indolent (slow-growing) types, and they can be formed from either B-cells or T-cells. B-cell non-Hodgkin lymphomas include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. T-cell non-Hodgkin lymphomas include mycosis fungoides, anaplastic large cell lymphoma, and precursor T-lymphoblastic lymphoma. Lymphomas that occur after bone marrow or stem cell transplantation are usually B-cell non-Hodgkin lymphomas. Prognosis and treatment depend on the stage and type of disease. Polynucleotides of the present invention may be used to treat NHL. Such treatment may comprise the expression of anti-CD20 antibodies capable of binding mature B lymphocytes, halting their growth and/or killing them.

Adverse events associated with ibritumomab treatment may include any of those listed in Table 7 of the FDA label (herein incorporated by reference in its entirety.) In some cases, such adverse events may be avoided by replacing ibritumomab treatment with treatment with one or more polynucleotides of the present invention.

Certain sequences encoding ibritumomab fragments, domains or heavy or light chains are given in Table 16. The table is not an exhaustive list and any fragment or portion of the sequence may be encoded in the ibritumomab polynucleotides of the invention.

TABLE 16
Table of Ibritumomab Sequences

| | | SEQ ID |
|---|---|---|
| Description | Sequence | NO. |
| Mouse Anti- | QAYLQQSGAELVRPGASVKMSCKASGYTFTSYNM | 71 |
| CD20 Heavy | HWVKQTPRQGLEWIGAIYPGNGDTSYNQKFKGKA | |
| chain | TLTVDKSSSTAYMQLSSLTSEDSAVYFCARVVYYS | |
| | NSYWYFDVWGTGTTVTVSAPSVYPLAPVCGDTTG | |
| | SSVTLGCLVKGYFPEPVTLTWNSGSLSSGVHTFPA | |
| | VLQSDLYTLSSSVTVTSSTWPSQSITCNVAHPASST | |
| | KVDKKIEPRGPTIKPCPPCKCPAPNLLGGPSVFIFPP | |
| | KIKDVLMISLSPIVTCVVVDVSEDDPDVQISWFVNN | |
| | VEVHTAQTQTHREDYNSTLRVVSALPIQHQDWMS | |
| | GKEFKCKVNNKDLPAPIERTISKPKGSVRAPQVYV | |
| | LPPPEEEMTKKQVTLTCMVTDFMPEDIYVEWTNN | |
| | GKTELNYKNTEPVLDSDGSYFMYSKLRVEKKNWV | |
| | ERNSYSCSVVHEGLHNHHTTKSFSR | |
| Mouse Anti- | QIVLSQSPAILSASPGEKVTMTCRASSSVSYMHWY | 72 |
| CD20 Light | QQKPGSSPKPWIYAPSNLASGVPARFSGSGSGTSYS | |
| chain | LTISRVEAEDAATYYCQQWSFNPPTFGAGTKLELK | |
| | RADAAPTVFIFPPSDEQLKSGTASVVCLLNNFYPRE | |
| | AKVQWKVDNALQSGNSQESVTEQDSKDSTYSLSS | |
| | TLTLSKADYEKHKVYACEVTHQGLSSPVTKSFN | |
| VH domain | MGWSLILLFLVAVATRVLSQVQLQQPGAELVKPG | 73 |
| (as translated) | ASVKMSCKASGYTFTSYNMHWVKQTPGRGLEWI | |
| listed in | GAIYPGNGDTSYNQKFKGKATLTADKSSSTAYMQ | |
| U.S. Pat. No. 5,736,137 | LSSLTSEDSAVYYCARSTYYGGDWYFNVWGAGTT | |
| | VTVSA | |
| VL domain (as | MDFQVQIISFLLISASVIMSRGQIVLSQSPAILSASPG | 74 |
| translated) | EKVTMTCRASSSVSYIHWFQQKPGSSPKPWIYATS | |
| listed in | NLASGVPVRFSGSGSGTSYSLTISRVEAEDAATYYC | |
| U.S. Pat. No. 5,736,137 | QQWTSNPPTFGGGTKLEIK | |
| VH domain | QVQLQQPGAELVKPGASVKMSCKASGYTFTSYNM | 75 |
| listed in | HWVKQTPGRGLEWIGAIYPGNGDTSYNQKFKGKA | |
| U.S. Pat. No. 5,736,137 | TLTADKSSSTAYMQLSSLTSEDSAVYYCARSTYYG | |
| (with signal | GDWYFNVWGAGTTVTVSA | |
| sequence | | |
| removed) | | |
| VL domain | QVQIISFLLISASVIMSRGQIVLSQSPAILSASPGEKV | 76 |
| listed in | TMTCRASSSVSYIHWFQQKPGSSPKPWIYATSNLA | |
| U.S. Pat. No. 5,736,137 | SGVPVRFSGSGSGTSYSLTISRVEAEDAATYYCQQ | |
| (with signal | WTSNPPTFGGGTKLEIK | |
| sequence | | |
| removed) | | |

According to the present invention, the ibritumomab polynucleotides of the present invention are engineered such that they may comprise one or more coding regions.

The coding regions of the ibritumomab polynucleotides may encode any of the regions or portions of the ibritumomab antibody. They may also further comprise coding regions not found in the original or parent ibritumomab antibody.

The ibritumomab polynucleotides of the present invention may be engineered to produce any of the 5 standard classes of immunoglobulins as shown in FIG. 1 using the ibritumomab antibody or any of its component parts as a starting molecule.

The ibritumomab polynucleotides may also be engineered according to the present invention to produce a variant ibritumomab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Infliximab Parent Molecule or Antibody

According to the present invention, infliximab polynucleotides or constructs and their associated infliximab compositions are designed to produce the infliximab antibody, a variant or a portion thereof in vivo.

Infliximab, also known as REMICADE® and Ig gamma-1 chain C region, is a chimeric IgG1-kappa monoclonal antibody (composed of human constant and murine variable regions) specific for human tumor necrosis factor-alpha (TNF-alpha) developed by Centocor Pharmaceuticals. Infliximab is produced by a recombinant cell line cultured by continuous perfusion and is purified by a series of steps that includes measures to inactivate and remove viruses. Infliximab is similar to the commercial antibody Golimumab, except that Golimumab has been engineering to be fully human.

Infliximab is a monoclonal antibody that attaches to, and blocks the action of TNF-alpha by inhibiting binding of TNF-alpha with its receptors. Infliximab can also able to limit the activation of neutrophil and eosinophil functional activity, can reduce the production of tissue degrading enzymes produced by synoviocytes and/or chondrocytes and can decrease synovitis and joint erosions in collagen-induced arthritis to allows eroded joints to heal.

However, some subjects have suffered from serious infections leading to hospitalization or death as a result of receiving Infliximab treatments. Therefore, there is a need in the art to develop a safer alternative the Infliximab antibody to a subject in order to treat the subject in need thereof.

In one embodiment, the polynucleotides described herein encode a chimeric IgG1-kappa monoclonal antibody (composed of human constant and murine variable regions) or fragments or variants thereof specific for human tumor necrosis factor-alpha (TNF-alpha). These polynucleotides can bind to and inhibit soluble and transmembrane human TNF-alpha. The inhibition of TNF-alpha can prevent the binding of TNF-alpha to its receptors which can prevent both leukocyte infiltration through prevention of cell adhesion proteins such as, but not limited to, E-selectin, ICAM-1 and VCAM-1, and pro-inflammatory cytokine secretion such as, but not limited to, IL-6, IL-8, G-CSF and GM-CSF. As a non-limiting example, the polynucleotide can encode Infliximab or a fragment or variant thereof.

In one embodiment, the polynucleotides described herein encode a chimeric IgG1-kappa monoclonal antibody (composed of human constant and murine variable regions) or fragments or variants thereof specific for human tumor necrosis factor-alpha (TNF-alpha) and these polynucleotides encode an antibody that does not bind to or neutralize other TNF superfamily ligands such as, but not limited to, lymphotoxin. As a non-limiting example, the polynucleotide can encode Infliximab or a fragment or variant thereof.

In one embodiment, the polynucleotides described herein encode a modulator of a target, such as, but not limited to, tumor necrosis factor (TNF). As a non-limiting example, the polynucleotide can encode Infliximab or a fragment or variant thereof which is known to modulate TNF-alpha in a subject. As a non-limiting example, the modulation of TNF-alpha may reduce the production of pro-inflammatory cytokines such as interleukin (IL) 1 and 6.

In one embodiment, the polynucleotides described herein may encode Infliximab or fragments or variants thereof and may be used to help reduce the symptoms of pain and inflammation associated with autoimmune diseases.

In one embodiment, the polynucleotides described herein may encode Infliximab or fragments or variants thereof and may be used to help manage the signs and symptoms and/or maintain clinical remission in those suffering from moderate to severe active Crohn's disease or ulcerative colitis. As a non-limiting example, the polynucleotides may be used to treat adults with moderate to severe active Crohn's disease or ulcerative colitis. As another non-limiting example, the polynucleotides may be used to treat children with moderate to severe active Crohn's disease or ulcerative colitis.

In one embodiment, the polynucleotides described herein may encode Infliximab or fragments or variants thereof and may be used to help manage the signs and symptoms in those suffering from rheumatoid arthritis, anklosing spondylitis, psoriatic arthritis and juvenile arthritis. As a non-limiting example, the polynucleotides may be used to inhibit the progression of structural damage caused by psoriatic arthritis.

In one embodiment, the polynucleotides described herein may encode Infliximab or a fragment or variant thereof. These polynucleotides may be used to treat a variety of diseases and/or disorders such as but not limited to inflammatory diseases. As a non-limiting example, the polynucleotides encoding Infliximab or a fragment or variant thereof may be used as a treatment for moderate to severe active rheumatoid arthritis (RA). The use of polynucleotides encoding Infliximab as a treatment for moderate to severe active rheumatoid arthritis (RA) may be an adjunct therapy to methotrexate treatment. As another non-limiting example, the polynucleotides encoding Infliximab or a fragment or variant thereof may be used as a treatment for active ankylosing spondylitis (AS). As yet another non-limiting example, the polynucleotides encoding Infliximab or a fragment or variant thereof may be used as a treatment for moderate to severe ulcerative colitis (UC).

In one embodiment, the polynucleotides described herein may encode Infliximab or a fragment or variant thereof may be used in the treatment of psoriasis, sarcoidosis, Behcet's Disease, Giant Cell Arthritis, Uveitis, SAPHO syndrome, Polychondritis, Sjogren's Syndrome, Celiac Disease, Toxic Epidermal Necrolysis, Subcorneal Pustular Dermatosis, Pyoderma Gangrenosum, Pulmonary Fibrosis, Juvenile Idiopathic Arthritis, Kawasaki Disease, Crohn's Disease and Ulcerative Colitis.

In one embodiment, prior to treatment with the polynucleotides described herein a subject may be tested for infections such as, but not limited to, tuberculosis (TB).

In one embodiment, the polynucleotides described herein may encode Infliximab or a fragment or variant thereof and may not cause moderate to severe heart failure.

In one embodiment, the polynucleotides described herein may encode Infliximab or a fragment or variant thereof and may not cause lymphoma or other types of cancer. As a non-limiting example, the cancer may be hepatosplenic T-cell lymphoma (HSTCL).

In one embodiment, the polynucleotides described herein encoding Infliximab or a fragment or variant thereof are formulated for intravenous administration. As a non-limiting example, the formulation may be an intravenous (IV) solution administered over 2 hours.

In one embodiment, the polynucleotides encoding Infliximab or a fragment or variant thereof has a half-life of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 day or at least 14 days.

In one embodiment, the dose of the polynucleotides encoding Infliximab or a fragment or variant thereof may be between 1 and 50 mg/kg, including, but not limited to, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg and 50 mg/kg.

In one embodiment, the polynucleotides described herein encoding Infliximab or a fragment or variant thereof are administered at Week 0, at Week 2, at Week 6 and either every eight weeks after Week 6 for most therapies or every six weeks after Week 6 for arthritis of the spine.

In one embodiment, the polynucleotides described herein encoding Infliximab or a fragment or variant thereof may be formulated for intravenous delivery for adults with Crohn's Disease. As a non-limiting example, the amount of drug administered may be 5 mg/kg given as an IV regimen at Week 0, Week 2 and Week 6 followed by a maintenance dose of 5 mg/kg every 8 weeks after Week 6. A dose of up to 10 mg/kg may be used as a maintenance dose for those who initially respond to treatment but then lose their response over time. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Crohn's Disease and may be co-administered with azathioprine. Approximately 2 days before the initial administration of Infliximab a dose of oral azathioprine (e.g., 2 to 2.5 mg/kg) may be administered to the subject receiving the intravenous delivery of the drug.

In one embodiment, the polynucleotides described herein encoding Infliximab or a fragment or variant thereof may be formulated for intravenous delivery for adults with Ulcerative Colitis. As a non-limiting example, the amount of drug administered may be 5 mg/kg given as an IV regimen at Week 0, Week 2 and Week 6 followed by a maintenance dose of 5 mg/kg every 8 weeks after Week 6. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Rheumatoid Arthritis. As a non-limiting example, the amount of drug administered may be 3 mg/kg given as an IV regimen at Week 0, Week 2 and Week 6 followed by a maintenance dose of 3 mg/kg every 8 weeks after Week 6. A dose of up to 10 mg/kg may be used as a maintenance dose as often as every 4 weeks for those who have an incomplete response. As a non-limiting example, the drug may be dissolved in saline (e.g., 250 mL of saline) for administration. For the treatment of Rheumatoid Arthritis, combination therapy with methotrexate may also be considered.

In one embodiment, the polynucleotides described herein encoding Infliximab or a fragment or variant thereof may be formulated for intravenous delivery for adults with Ankylosing Spondylitis. As a non-limiting example, the amount of drug administered may be 5 mg/kg given as an IV regimen at Week 0, Week 2 and Week 6 followed by a maintenance dose of 5 mg/kg every 6 weeks after Week 6. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Psoriatic Arthritis. As a non-limiting example, the amount of drug administered may be 5 mg/kg given as an IV regimen at Week 0, Week 2 and Week 6 followed by a maintenance dose of 3-5 mg/kg every 8 weeks after Week 6. As a non-limiting example, the drug may be dissolved in saline (e.g., 250 mL of saline) for administration. For the treatment of Psoriatic Arthritis, combination therapy with methotrexate may also be considered.

In one embodiment, the polynucleotides described herein encoding Infliximab or a fragment or variant thereof may be formulated for intravenous delivery for adults with Psoriasis. As a non-limiting example, the amount of drug administered may be 5 mg/kg given as an IV regimen at Week 0, Week 2 and Week 6 followed by a maintenance dose of 5 mg/kg every 8 weeks after Week 6. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Sarcoidosis. As a non-limiting example, the amount of drug administered may be 5 mg/kg given as an IV regimen at Week 0, Week 2 and every 4 to 8 weeks thereafter. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Behcet's Disease. As a non-limiting example, the amount of drug administered may be 5 mg/kg given over 3 hours as an IV regimen at Week 0, Week 2 and Week 6. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Giant Cell Arteritis. As a non-limiting example, the amount of drug administered may be 3 mg/kg given over 2 hours as an IV regimen at Week 0, Week 2 and Week 6. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Uveitis. As a non-limiting example, the amount of drug administered may be 5 mg/kg given as an IV regimen at Week 0, Week 2 and Week 6 followed by a maintenance dose of 5 mg/kg every 2 months after Week 6. As another non-limiting example, the amount of drug administered may be a single dose of 3 mg/kg given over 3 hours as an IV. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with SAPHO syndrome. As a non-limiting example, the amount of drug administered may be 3 mg/kg given as an IV regimen every 4 weeks. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Polychondritis. As a non-limiting example, the amount of drug administered may be 5 mg/kg given as an IV regimen at Week 0, Week 2, Week 6, Week 14, Week 22, Week 30 and Week 38. As a non-limiting example, the drug may be dissolved in saline (e.g., 250 mL of saline) for administration. For the treatment of Polychondritis, combination therapy with oral prednisone may also be considered.

In one embodiment, the polynucleotides described herein encoding Infliximab or a fragment or variant thereof may be formulated for intravenous delivery for adults with Sjogren's Syndrome. As a non-limiting example, the amount of drug administered may be 3 mg/kg given as an IV regimen at Week 0, Week 2 and Week 6. As another non-limiting example, the amount of drug administered may be 3 mg/kg given as an IV at Week 0, Week 2 and Week 6 and every 12 weeks after Week 6. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Celiac Disease. As a non-limiting example, the amount of drug administered over 2 hours may be 5 mg/kg given as a single dose. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Toxic Epidermal Necrolysis. As a non-limiting example, the amount of drug administered over 2 hours may be 5 mg/kg given as a single dose. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Subcorneal Pustular Dermatosis. As a non-limiting example, the amount of drug administered over 2 hours may be 5 mg/kg given as a single dose. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Pyoderma Gangrenosum. As a non-limiting example, the amount of drug administered may be 5 mg/kg given as an IV regimen at Week 0, Week 2, Week 4, Week 8 and Week 10 and then a maintenance dose of 5 mg/kg every 6 to 8 weeks thereafter. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for adults with Pulmonary Fibrosis. As a non-limiting example, the amount of drug administered may be 3 mg/kg given as an IV regimen at Week 0, Week 2, Week 4 and Week 6 and then a maintenance dose of 3 mg/kg every other 8 weeks thereafter. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for children 6 years or older with Acute Crohn's Disease. As a non-limiting example, the amount of drug administered may be 5 mg/kg given as an IV regimen at Week 0, Week 2, and Week 6 and then a maintenance dose of 5 mg/kg every 8 weeks thereafter. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for children 6 years or older with Crohn's Disease as a maintenance therapy. As a non-limiting example, the amount of drug administered may be 5 mg/kg given as an IV regimen at Week 0, Week 2, and Week 6 and then a maintenance dose of 5 mg/kg every 8 weeks thereafter. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for children 6 years or older with Ulcerative Colitis. As a non-limiting example, the amount of drug administered may be 5 mg/kg given as an IV regimen at Week 0, Week 2, and Week 6 and then a maintenance dose of 5 mg/kg every 8 weeks thereafter. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for children 10 years or older with Juvenile Idiopathic Arthritis. As a non-limiting example, the amount of drug administered may be 3 mg/kg given as an IV regimen at Week 0, Week 2, and Week 6 and then a maintenance dose of 3 mg/kg every 8 weeks thereafter. As a non-limiting example, the drug may be dissolved in saline (e.g., 250 mL of saline) for administration.

In one embodiment, the polynucleotides described herein encoding Infliximab or a fragment or variant thereof may be formulated for intravenous delivery for children 3 years or older with Kawasaki Disease. As a non-limiting example, the amount of drug administered may be 5 mg/kg given as an IV regimen at Day 0, Day 45, Day 59 and Day 89. As a non-limiting example, the drug may be dissolved in saline (e.g., 250 mL of saline) for administration.

In one embodiment, the polynucleotides described herein may encode an antibody that binds to an epitope of at least 5 amino acid residues 87-108 or both of residues 59-80 and 87-108 of hTNFc of SEQ ID NO: 1 of International Patent Application WO1992016553 (the contents of which is herein incorporated by reference in its entirety), but which do not bind known or putative receptor binding portions of TNF, such as amino acid sequences 1-20, 11-13, 37-42, 49-57 or 155-157 of TNF of SEQ ID NO: 1 of International Patent Application WO1992016553.

TABLE 17
Table of Infliximab Sequences

| Description | Sequence | SEQ ID NO |
|---|---|---|
| Heavy Chain Variable Region | EVKLEESGGGLVQPGGSMKLSC | 77 |
| (See Int'l Patent Pub | VASGFIFSNHWMNWVRQSPEKG | |
| WO2013087911 SEQ ID NO: 2; | LEWVAEIRSKSINSATHYAESVK | |
| the contents of which is herein | GRFTISRDDSKSAVYLQMTDLRT | |
| incorporated by reference in its | EDTGVYYCSRNYYGSTYDYWGQ | |
| entirety) | GTTLTVSS | |
| Light Chain Variable Region | DILLTQSPAILSVSPGERVSFSCRA | 78 |
| (See Int'l Patent Pub | SQFVGSSIHWYQQRTNGSPRLLIK | |
| WO2013087911 SEQ ID NO: 3; | YASESMSGIPSRFSGSGSGTDFTL | |
| the contents of which is herein | SINTVESEDIADYYCQQSHSWPFT | |
| incorporated by reference in its | FGSGTNLEVK | |
| entirety) | | |

Certain sequences encoding infliximab fragments, domains or heavy or light chains are given in Table 17. The table is not an exhaustive list and any fragment or portion of the sequence may be encoded in the infliximab polynucleotides of the invention.

According to the present invention, the infliximab polynucleotides of the present invention are engineered such that they may comprise one or more coding regions.

The coding regions of the infliximab polynucleotides may encode any of the regions or portions of the infliximab antibody. They may also further comprise coding regions not found in the original or parent infliximab antibody.

The infliximab polynucleotides of the present invention may be engineered to produce any of the 5 standard classes of immunoglobulins as shown in FIG. 1 using the infliximab antibody or any of its component parts as a starting molecule.

The infliximab polynucleotides may also be engineered according to the present invention to produce a variant infliximab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Ipilimumab Parent Molecule or Antibody

According to the present invention, ipilimumab polynucleotides or constructs and their associated ipilimumab compositions are designed to produce the ipilimumab antibody, a variant or a portion thereof in vivo.

Ipilimumab, also known as YERVOY®, Anti-cytotoxic T-lymphocyte-associated antigen-4 monoclonal antibody, MOAB CTLA-4, monoclonal antibody CTLA-4, BMS-734016, MDX-010 and MDX-CTLA-4, is a monoclonal antibody directed against cytotoxic T-lymphocyte-associated antigen-4 (CTLA4), an antigen that is expressed on activated T-cells and exhibits affinity for B7 co-stimulatory molecules that was developed by Bristol-Myers Squibb. Ipilimumab is an immunoglobulin G1 (IgG1)—kappa immunoglobulin produced in mammalian (Chinese hamster ovary) cell culture.

Ipilimumab is a monoclonal antibody that helps the immune system recognize and kill cancer cells because by binding to CTLA4. Ipilimumab enhances T-cell activation and can block B7-1 and B7-2 T-cell co-stimulatory pathways.

However, some subjects have suffered from severe and fatal immune-mediated adverse reactions as a result of receiving ipilimumab treatments. Therefore, there is a need in the art to develop a safer alternative to delivering the ipilimumab antibody to a subject in order to treat the subject in need thereof.

In one embodiment, the polynucleotides described herein encode a monoclonal antibody directed against cytotoxic T-lymphocyte-associated antigen-4 (CTLA4) or fragments or variants thereof. The monoclonal antibody may be a fully human immunoglobulin (IgG1-kappa) consisting of four polypeptide chains; two identical heavy chains primarily consisting of 447 amino acids each with two identical kappa light chains consisting of 215 amino acids each linked through inter-chain disulfide bonds.

In one embodiment, the polynucleotides described herein encode a monoclonal antibody directed against cytotoxic T-lymphocyte-associated antigen-4 (CTLA4) or fragments or variants thereof such as, but not limited to, ipilimumab. The polynucleotides can activate the immune system by targeting CTLA-4 which is found on the surface of T cells. By blocking the interaction of CTLA-4 with its ligands (e.g., CD80 and CD86) these polynucleotides can lead to the activation and spread of T cells which can infiltrate tumor and kill tumor cells.

Certain sequences encoding ipilimumab fragments, domains or heavy or light chains are given in Table 18. The table is not an exhaustive list and any fragment or portion of the sequence may be encoded in the ipilimumab polynucleotides of the invention.

In one embodiment, the polynucleotides described herein may encode at least one of the amino acid sequences, or a portion thereof, described in Table 18. In another embodiment, the polynucleotides described herein may be similar (percent homologous) to any of the nucleic acid sequences described in Table 18.

TABLE 18
Table of Ipilimumab Sequences

| | | SEQ |
|---|---|---|
| | | ID |
| Description | Sequence | NO |
| Heavy Chain Variable Region | CAGGTGCAGCTGGTGGAGTCTGGGG | 79 |
| (See Int'l Patent Pub | GAGGCGTGGTCCAGCCTGGGAGGTC | |
| WO2001014424 SEQ ID NO: 16, | CCTGAGACTCTCCTGTGCAGCCTCT | |
| FIG. 6; the contents of which is | GGATTCACCTTCAGTAGCTATACTA | |
| herein incorporated by reference | TGCACTGGGTCCGCCAGGCTCCAGG | |
| in its entirety) | CAAGGGGCTGGAGTGGGTGACATTT | |
| | ATATCATATGATGGAAACAATAAAT | |
| | ACTACGCAGACTCCGTGAAGGGCCG | |
| | ATTCACCATCTCCAGAGACAATTCC | |
| | AAGAACACGCTGTATCTGCAAATGA | |
| | ACAGCCTGAGAGCTGAGGACACGG | |
| | CTATATATTACTGTGCGAGGACCGG | |
| | CTGGCTGGGGCCCTTTGACTACTGG | |
| | GGCCAGGGAACCCTGGTCACCGTCT | |
| | CCTCAG | |
| Heavy Chain Variable Region | QVQLVESGGGVVQPGRSLRLSCAASG | 80 |
| (See Int'l Patent Pub | FTFSSYTMHWVRQAPGKGLEWVTFIS | |
| WO2001014424 SEQ ID NO: 17, | YDGNNKYYADSVKGRFTISRDNSKNT | |
| FIG. 8; the contents of which is | LYLQMNSLRAEDTAIYYCARTGWLG | |
| herein incorporated by reference | PFDYWGQGTLVTVSS | |
| in its entirety) | | |
| Light Chain Variable Region (See | GAAATTGTGTTGACGCAGTCTCCAG | 81 |
| Int'l Patent Pub WO2001014424 | GCACCCTGTCTTTGTCTCCAGGGGA | |
| SEQ ID NO: 6, FIG. 5; the | AAGAGCCACCCTCTCCTGCAGGGCC | |
| contents of which is herein | AGTCAGAGTGTTGGCAGCAGCTACT | |
| incorporated by reference in its | TAGCCTGGTACCAGCAGAAACCTGG | |
| entirety) | CCAGGCTCCCAGGCTCCTCATCTAT | |
| | GGTGCATTCAGCAGGGCCACTGGCA | |
| | TCCCAGACAGGTTCAGTGGCAGTGG | |
| | GTCTGGGACAGACTTCACTCTCACC | |
| | ATCAGCAGACTGGAGCCTGAAGATT | |
| | TTGCAGTGTATTACTGTCAGCAGTA | |
| | TGGTAGCTCACCGTGGACGTTCGGC | |
| | CAAGGGACCAAGGTGGAAATCAAA | |
| | C | |
| Light Chain Variable Region (See | EIVLTQSPGTLSLSPGERATLSCRASQS | 82 |
| Int'l Patent Pub WO2001014424 | VGSSYLAWYQQKPGQAPRLLIYGAFS | |
| SEQ ID NO: 7, FIG. 7; the | RATGIPDRFSGSGSGTDFTLTISRLEPE | |
| contents of which is herein | DFAVYYCQQYGSSPWTFGQGTKVEIK | |
| incorporated by reference in its | | |
| entirety) | | |
| Heavy Chain CDR1 (See Int'l | SYTMH | 83 |
| Patent Pub WO2001014424 SEQ | | |
| ID NO: 27; the contents of which | | |
| is herein incorporated by | | |
| reference in its entirety) | | |
| Heavy Chain CDR2 (See Int'l | FISYDGNNKYYADSVKG | 84 |
| Patent Pub WO2001014424 SEQ | | |
| ID NO: 32; the contents of which | | |
| is herein incorporated by | | |
| reference in its entirety) | | |
| Heavy Chain CDR3 (See Int'l | TGWLGPFDY | 85 |
| Patent Pub WO2001014424 SEQ | | |
| ID NO: 37; the contents of which | | |
| is herein incorporated by | | |
| reference in its entirety) | | |
| Light Chain CDR1 (See Int'l | RASQSVGSSYLA | 86 |
| Patent Pub WO2001014424 SEQ | | |
| ID NO: 24; the contents of which | | |
| is herein incorporated by | | |
| reference in its entirety) | | |
| Light Chain CDR2 (See Int'l | GAFSRAT | 87 |
| Patent Pub WO2001014424 SEQ | | |
| ID NO: 29; the contents of which | | |
| is herein incorporated by | | |
| reference in its entirety) | | |
| Light Chain CDR3 (See Int'l | QQYGSSPWT | 88 |
| Patent Pub WO2001014424 SEQ | | |
| ID NO: 35; the contents of which | | |
| is herein incorporated by | | |
| reference in its entirety) | | |

In one embodiment, the polynucleotides described herein encode a modulator of a target, such as, but not limited to, cytotoxic T-lymphocyte-associated antigen-4 (CTLA-4). As a non-limiting example, the polynucleotide can encode ipilimumab or a fragment or variant thereof which is known to modulate CTLA-4 in a subject.

In one embodiment, the polynucleotides described herein may encode ipilimumab or fragments or variants thereof and may be used as a therapy for melanoma.

In one embodiment, the polynucleotides described herein may encode ipilimumab or fragments or variants thereof and may be used as a therapy for cancer such as, but not limited to, non-small cell lung carcinoma (NSCLC), small cell lung cancer (SCLC) and metastatic hormone-refractory prostate cancer.

In one embodiment, prior to and during treatment with the polynucleotides described herein a subject may be tested for to determine liver and thyroid function.

In one embodiment, the polynucleotides described herein encoding ipilimumab or a fragment or variant thereof are formulated for intravenous administration. As a non-limiting example, the formulation may be an intravenous (IV) solution administered over 90 minutes.

In one embodiment, the polynucleotides encoding ipilimumab or a fragment or variant thereof has a half-life of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 day or at least 14 days.

In one embodiment, the dose of the polynucleotides encoding ipilimumab or a fragment or variant thereof may be between 1 and 50 mg/kg, including, but not limited to, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg and 50 mg/kg. As a non-limiting example, the dose of the polynucleotides encoding ipilimumab or a fragment or variant thereof is 3 mg/kg.

In one embodiment, the dose of the polynucleotides encoding ipilimumab or a fragment or variant thereof may be 5 mg/mL. As a non-limiting example, the dose may be 50 mg of drug in 10 mL of solution or 200 mg of drug in 40 mL of solution.

In one embodiment, the polynucleotides described herein encoding ipilimumab or a fragment or variant thereof are administered four times with three weeks in between each dose.

In one embodiment, the polynucleotides described herein encoding ipilimumab or a fragment or variant thereof are co-administered with a corticosteroid such as, but not limited to, prednisone. The co-administration of the corticosteroid may be used to alleviate any unwanted side-effects from administration of the polynucleotides encoding ipilimumab or a fragment or variant thereof.

In one embodiment, the polynucleotides described herein encoding ipilimumab or a fragment or variant thereof may be administered over 90 minutes through an IV line containing a sterile, nonpyogenic, low-protein binding inline filter. Following each administration of ipilimumab, the IV line may be flushed with 0.9% sodium chloride or 5% dextrose.

In one embodiment, the polynucleotides described herein encoding ipilimumab or a fragment or variant thereof may be formulated for intravenous delivery for adults with melanoma.

In one embodiment, the polynucleotides described herein encoding ipilimumab or a fragment or variant thereof may be formulated for intravenous delivery for adults with non-small cell lung carcinoma (NSCLC), small cell lung cancer (SCLC) or metastatic hormone-refractory prostate cancer.

According to the present invention, the ipilimumab polynucleotides of the present invention are engineered such that they may comprise one or more coding regions.

The coding regions of the ipilimumab polynucleotides may encode any of the regions or portions of the ipilimumab antibody. They may also further comprise coding regions not found in the original or parent ipilimumab antibody.

The ipilimumab polynucleotides of the present invention may be engineered to produce any of the 5 standard classes of immunoglobulins as shown in FIG. 1 using the ipilimumab antibody or any of its component parts as a starting molecule.

The ipilimumab polynucleotides may also be engineered according to the present invention to produce a variant ipilimumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Muromonab Parent Molecule or Antibody

According to the present invention, muromonab polynucleotides or constructs and their associated muromonab compositions are designed to produce the muromonab antibody, a variant or a portion thereof in vivo.

Muromonab-CD3, also known as ORTHOCLONE OKT3®, muromonab and AntiCD3, is a murine monoclonal antibody directed against the CD3 (T3) receptor on the surface of human T-cells (T-lymphocytes) cultured using the murine ascites method. Muromonab-CD3 is 93% monomeric immune globulin G type 2a (IgG2a). Muromonab-CD3 may be effective in the treatment of allograft rejection.

While not wishing to be bound by theory, once administered muromonab-CD3 may act to block the function of mature T lymphocytes or it may modulate the T lymphocyte antigen receptor-CD-3 complex of circulating T lymphocytes. (See e.g., Hooks et al. Pharmacotherapy 1991. 11(1), 26-27; the contents of which are herein incorporated by reference in its entirety).

However, some subjects have suffered from acute toxicity and some life threatening reactions. Therefore, there is a need in the art to develop a safer alternative to delivering the muromonab-CD3 antibody to a subject in order to treat the subject in need thereof.

In one embodiment, the polynucleotides described herein encode a monoclonal antibody that can bind to the T-cell surface glycoprotein CD3 epsilon chain. While not wishing to be bound by theory, the binding may kill CD-3 positive cells by inducing Fc mediated apoptosis, antibody mediated cytotoxicity and complement-dependent cytotoxicity. As a non-limiting example, the monoclonal antibody that can bind to the T-cell surface glycoprotein CD3 epsilon chain is muromonab-CD3.

In one embodiment, the polynucleotides described herein encode a modulator of a target, such as, but not limited to T-cell surface glycoprotein CD3 delta chain. As a non-limiting example, the polynucleotide can encode muromonab-CD3 or a fragment or variant thereof which is known to modulate T-cell surface glycoprotein CD3 delta chain in a subject.

In one embodiment, the polynucleotides described herein encode a modulator of a target, such as, but not limited to T-cell surface glycoprotein CD3 epsilon chain. As a non-limiting example, the polynucleotide can encode muromonab-CD3 or a fragment or variant thereof which is known to modulate T-cell surface glycoprotein CD3 epsilon chain in a subject.

In one embodiment, the polynucleotides described herein encode a modulator of a target, such as, but not limited to T-cell surface glycoprotein CD3 gamma chain. As a non-limiting example, the polynucleotide can encode muromonab-CD3 or a fragment or variant thereof which is known to modulate T-cell surface glycoprotein CD3 gamma chain in a subject.

In one embodiment, the polynucleotides described herein encode a modulator of a target, such as, but not limited to T-cell surface glycoprotein CD3 zeta chain. As a non-limiting example, the polynucleotide can encode muromonab-CD3 or a fragment or variant thereof which is known to modulate T-cell surface glycoprotein CD3 zeta chain in a subject.

In one embodiment, the polynucleotides described herein encode a modulator of a target, such as, but not limited to low affinity immunoglobulin gamma Fc region receptor III-B (FCGR3B). As a non-limiting example, the polynucleotide can encode muromonab-CD3 or a fragment or variant thereof which is known to modulate FCGR3B in a subject.

In one embodiment, the polynucleotides described herein encode a modulator of a target, such as, but not limited to Complement C1r subcomponent (C1R). As a non-limiting example, the polynucleotide can encode muromonab-CD3 or a fragment or variant thereof which is known to modulate C1R in a subject.

In one embodiment, the polynucleotides described herein encode a modulator of a target, such as, but not limited to Complement C1q subcomponent subunit A (C1QA). As a non-limiting example, the polynucleotide can encode muromonab-CD3 or a fragment or variant thereof which is known to modulate C1QA in a subject.

In one embodiment, the polynucleotides described herein encode a modulator of a target, such as, but not limited to Complement C1q subcomponent subunit B (C1QB). As a non-limiting example, the polynucleotide can encode muromonab-CD3 or a fragment or variant thereof which is known to modulate C1QB in a subject.

In one embodiment, the polynucleotides described herein encode a modulator of a target, such as, but not limited to Complement C1q subcomponent subunit B (C1QC). As a non-limiting example, the polynucleotide can encode muromonab-CD3 or a fragment or variant thereof which is known to modulate C1QC in a subject.

In one embodiment, the polynucleotides described herein encode a modulator of a target, such as, but not limited to Low affinity immunoglobulin gamma Fc region receptor III-A (FCGR3A). As a non-limiting example, the polynucleotide can encode muromonab-CD3 or a fragment or variant thereof which is known to modulate FCGR3A in a subject.

In one embodiment, the polynucleotides described herein encode a modulator of a target, such as, but not limited to Complement C1s subcomponent (C1s). As a non-limiting example, the polynucleotide can encode muromonab-CD3 or a fragment or variant thereof which is known to modulate C15 in a subject.

In one embodiment, the polynucleotides described herein encode a modulator of a target, such as, but not limited to High affinity immunoglobulin gamma Fc receptor I (FCGR1A). As a non-limiting example, the polynucleotide can encode muromonab-CD3 or a fragment or variant thereof which is known to modulate FCGR1A in a subject.

In one embodiment, the polynucleotides described herein encode a modulator of a target, such as, but not limited to Low affinity immunoglobulin gamma Fc region receptor II-a (FCGR2A). As a non-limiting example, the polynucleotide can encode muromonab-CD3 or a fragment or variant thereof which is known to modulate FCGR2A in a subject.

In one embodiment, the polynucleotides described herein encode a modulator of a target, such as, but not limited to Low affinity immunoglobulin gamma Fc region receptor II-b (FCGR2B). As a non-limiting example, the polynucleotide can encode muromonab-CD3 or a fragment or variant thereof which is known to modulate FCGR2B in a subject.

In one embodiment, the polynucleotides described herein encode a modulator of a target, such as, but not limited to Low affinity immunoglobulin gamma Fc region receptor II-c (FCGR2C). As a non-limiting example, the polynucleotide can encode muromonab-CD3 or a fragment or variant thereof which is known to modulate FCGR2C in a subject.

Certain sequences encoding muromonab fragments, domains or heavy or light chains are given in Table 19. The table is not an exhaustive list and any fragment or portion of the sequence may be encoded in the muromonab polynucleotides of the invention.

TABLE 19
Table of Muromonab Sequences

| | | SEQ |
|---|---|---|
| | | ID |
| Description | Sequence | NO |
| Heavy Chain | QVQLQQSGAELARPGASVKMSCKASGYTFT | 89 |
| | RYTMHWVKQRPGQGLEWIGYINPSRGYTNY | |
| | NQKFKDKATLTTDKSSSTAYMQLSSLTSEDS | |
| | AVYYCARYYDDHYCLDYWGQGTTLTVSSA | |
| | KTTAPSVYPLAPVCGGTTGSSVTLGCLVKGY | |
| | FPEPVTLTWNSGSLSSGVHTFPAVLQSDLYTL | |
| | SSSVTVTSSTWPSQSITCNVAHPASSTKVDKK | |
| | IEPRPKSCDKTHTCPPCPAPELLGGPSVFLFPP | |
| | KPKDTLMISRTPEVTCVVVDVSHEDPEVKFN | |
| | WYVDGVEVHNAKTKPREEQYNSTYRVVSV | |
| | LTVLHQDWLNGKEYKCKVSNKALPAPIEKTI | |
| | SKAKGQPREPQVYTLPPSRDELTKNQVSLTC | |
| | LVKGFYPSDIAVEWESNGQPENNYKTTPPVL | |
| | DSDGSFFLYSKLTVDKSRWQQGNVFSCSVM | |
| | HEALHNHYTQKSLSLSPGK | |
| Light Chain | QIVLTQSPAIMSASPGEKVTMTCSASSSVSYM | 90 |
| | NWYQQKSGTSPKRWIYDTSKLASGVPAHFR | |
| | GSGSGTSYSLTISGMEAEDAATYYCQQWSSN | |
| | PFTFGSGTKLEINRADTAPTVSIFPPSSEQLTS | |
| | GGASVVCFLNNFYPKDINVKWKIDGSERQN | |
| | GVLNSWTDQDSKDSTYSMSSTLTLTKDEYE | |
| | RHNSYTCEATHKTSTSPIVKSFNRNEC | |

In one embodiment, the polynucleotides described herein may encode muromonab-CD3 or fragments or variants thereof and may be used as a therapy for organ transplant recipients in order to treat and/or prevent organ rejection. As a non-limiting example, the organ transplant may be kidney, liver, cardiac, pancreatic, bone marrow transplant (See e.g., Hooks et al. Pharmacotherapy 1991. 11(1), 26-27; the contents of which are herein incorporated by reference in its entirety).

In one embodiment, the polynucleotides described herein may encode muromonab-CD3 or fragments or variants thereof and may be used as an immunosuppressant. As a non-limiting example, the polynucleotides encoding muromonab-CD3 may block the action of certain blood cells (e.g., T lymphocytes) that can cause the body to reject a transplanted organ.

In one embodiment, the polynucleotides described herein encoding muromonab-CD3 or a fragment or variant thereof are formulated for intravenous administration. As a non-limiting example, the formulation may be an intravenous (IV) bolus solution administered over 2 minutes. As another non-limiting example, the dose of Muromoab-CD3 may be 5 mg administered as an IV bolus over 2 minutes daily for 10-14 days.

In one embodiment, the polynucleotides encoding muromonab-CD3 or a fragment or variant thereof has a half-life of at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 2 hours, at least 3 hours, at least 4 hours, at least 5 hours, at least 6 hours, at least 7 hours, at least 8 hours, at least 9 hours, at least 10 hour, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days or at least 7 days.

In one embodiment, the dose of the polynucleotides encoding muromonab-CD3 or a fragment or variant thereof may be between 1 and 50 mg/kg, including, but not limited to, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg and 50 mg/kg.

In one embodiment, if the subject is greater than 30 kg over the ideal body weight (IBW) the daily dose of polynucleotides encoding muromonab-CD3 is 5.0 mg by IV.

In one embodiment, if the subject is within 30 kg of the IBW the daily dose of polynucleotides encoding muromonab-CD3 is 2.5 mg by IV.

In one embodiment, the dose of polynucleotides encoding muromonab-CD3 produces serum levels of approximately 1000 mg/ml. According to Hooks et al. serum levels of approximately 1000 mg/ml should cause modulation of T lymphocytes in the circulation (See e.g., Hooks et al. Pharmacotherapy 1991. 11(1), 31; the contents of which are herein incorporated by reference in its entirety).

In one embodiment, about 1-4 hours prior to administration of the polynucleotides encoding muromonab-CD3 methylprednisilone sodium succinate is administered by IV at a dose of 8 mg/kg. Approximately 30 minutes after the administration of the polynucleotides encoding muromonab-CD3 about 100 mg of hydrocortisone sodium succinate is administered by IV. The administration of methylprednisolone sodium succinate and/or hydrocortisone sodium succinate may help decrease the incidence and severity of a first dose reaction.

According to the present invention, the muromonab polynucleotides of the present invention are engineered such that they may comprise one or more coding regions.

The coding regions of the muromonab polynucleotides may encode any of the regions or portions of the muromonab antibody. They may also further comprise coding regions not found in the original or parent muromonab antibody.

The muromonab polynucleotides of the present invention may be engineered to produce any of the 5 standard classes of immunoglobulins as shown in FIG. 1 using the muromonab antibody or any of its component parts as a starting molecule.

The muromonab polynucleotides may also be engineered according to the present invention to produce a variant muromonab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Natalizumab Parent Molecule or Antibody

According to the present invention, natalizumab polynucleotides or constructs and their associated natalizumab compositions are designed to produce the natalizumab antibody, a variant or a portion thereof in vivo.

Natalizumab is co-marketed, under the trade name Tysabri (formerly Antegren), by Elan Pharmaceuticals and Biogen Idec. Inc. This antibody also has been referred to as anti-alpha4 integrin or anti-VLA4 antibody. Natalizumab was initially approved by the Food and Drug Administration in November, 2004, but was voluntarily withdrawn from U.S. market in February, 2005 because of the risk of progressive multifocal leukoencephalopathy (PML). It was returned to market July, 2006 as monotherapy for the treatment of patients with relapsing forms of multiple sclerosis (MS).

Natalizumab is a humanized monoclonal antibody that binds to alpha4-integrin that mediates adhesion and migration of immune cells through interaction with its ligand, vascular cell adhesion molecule (VCAM)-1.

Natalizumab is used to prevent episodes of symptoms and slow the worsening of disability in patients with relapsing forms of multiple sclerosis (MS) (Niino M, et al., Ann Neurol., 2006, 59(5), 748-754; the content of which is herein incorporated by reference in its entirety). Natalizumab is also used to treat and prevent episodes of symptoms in people who have Crohn's disease (CD) but have not been helped by other medications or who cannot take other medications.

However, natalizumab can increase the risk of developing PML, a rare brain infection that usually causes death and disability in patients, and causes other side effects in patients, particularly in those who have compromised immune system.

Natalizumab is a recombinant humanized IgG4k monoclonal antibody produced in murine myeloma cells. Natalizumab contains human framework regions and the complementarity-determining regions (CDRs) of a murine antibody that binds to alpha4-integrin. The molecular weight of natalizumab is 149 KD.

Natalizumab was first described in U.S. Pat. No. 5,840,299 by Bendig et al (also as described in U.S. Pat. No. 6,033,665; and PCT patent publication NO: WO1997018838; the content of each of which are herein incorporated by reference in their entirety).

Natalizumab is a humanized version of murine monoclonal antibody 21.6. The three complementary determining regions (CDR1, CDR 2 and CDR3) of humanized light chain have amino acid sequences are derived from the corresponding CDRs of the mouse immunoglobulin light chain variable regions and a variable region framework from a human kappa light chain variable framework sequence. Similarly, the three complementary determining regions (CDR1, CDR 2 and CDR3) of humanized heavy chain have amino acid sequences from the corresponding CDRs of the mouse immunoglobulin heavy chain variable regions and a variable region framework from a human heavy chain variable framework sequence. The constant region(s) are substantially from a human immunoglobulin. Table 20 lists the amino acid sequences of heavy and light chain variable regions of natalizumab.

Certain sequences encoding natalizumab fragments, domains or heavy or light chains are given in Table 20. The table is not an exhaustive list and any fragment or portion of the sequence may be encoded in the natalizumab polynucleotides of the invention.

TABLE 20
Table of Natalizumab Sequences

| | | | SEQ |
|---|---|---|---|
| | | | ID |
| Description | Sequence | Source | NO. |
| The mouse 21-6 | MKCSWVMFFLMAVVTGVNSQVQLQ | From U.S. Pat. No. 5,840,299 | 91 |
| I heavy chain | QSGAELVKPGASVKLSCTASGFNIKD | SEQ ID NO: 4 | |
| variable region | TYIHCVKQRPEQGLEWIGRIDPANGY | | |
| for humanized | TKYDPKFQGKATITADTSSNTAYLQL | | |
| heavy chain | SSLTSEDTAVYFCAREGYYGNYGVY | | |
| | AMDYWGQGTSVTV | | |
| The mouse 21-6 | MRPSIQFLGLLLFWLHGAQCDIQMTQ | From U.S. Pat. No. 5,840,299 | 92 |
| I light chain | SPSSLSASLGGKVTITCKTSQDINKYM | SEQ ID NO: 2 | |
| variable region | AWYQHKPGKRPRLLIHYTSALQPGIPS | | |
| for humanized | RFSGSGSGRDYSFNISNLQPQDIATYY | | |
| light chain | CLQYDNLWTFGGGTKLQIK | | |
| Humanized | QVQLVQSGAQVKKPGASVKVSCKAS | From U.S. Pat. No. 5,840,299 | 93 |
| heavy chain | GYTFTSYAMHWVRQAPGQRLQWMG | SEQ ID NO: 10 | |
| variable region | WINAGNGNTKYSQKFQGRVTITRDTS | (21/28′CL | |
| framework | ASTAYMELSSLRSEDTAVYYCARGG | variable region | |
| | YYGSGSNYWGQGTLVTVSS | framework | |
| | | sequence) | |
| Humanized light | DIQMTQSPSSLSASVGDRVTITCQASQ | From U.S. Pat. No. 5,840,299 | 94 |
| chain variable | DIIKYLNWYQQTPGKAPKLLIYEASNL | SEQ ID NO: 6 | |
| region | QAGVPSRFSGSGSGTDYTFTISSLQPE | (REI variable | |
| framework | DIATYCQQYQSLPYTFGQGTKLQIT | region framework | |
| | | sequence) | |
| Mature heavy | QVQLVQSGAEVKKPGASVKVSCKAS | From U.S. Pat. No. 5,840,299 | 95 |
| chain variable | GFNIKDTYIHWVRQAPGQRLEWMGRI | SEQ ID NO: 11 | |
| sequence Ha | DPANGYTKYDPKFQGRVTITADTSAS | | |
| | TAYMELSSLRSEDTAVYYCAREGYY | | |
| | GNYGVYAMDYWGQGTLVTVSS | | |
| Mature heavy | QVQLVQSGAEVKKPGASVKVSCKAS | From U.S. Pat. No. 5,840,299 | 96 |
| chain variable | GFNIKSTAMHWVRQAPGQGLEWMG | SEQ ID NO: 12 | |
| sequence Hb | WINAGNGNTKYSQKFQGRVTITADTS | | |
| | ASTAYMELSSLRSEDTAVYYCARGG | | |
| | YYGSGSNYWGQGTLVTVSS | | |
| Mature heavy | QVQLVQSGAEVKKPGASVKVSCKAS | From U.S. Pat. No. 5,840,299 | 97 |
| chain variable | GFNIKSYAMHWVRQAPGQRLEWMG | SEQ ID NO: 13 | |
| sequence Hc | WINAGNGNTKYSQKFQGRVTITADTS | | |
| | ASTAYMELSSLRSEDTAVYYCARGG | | |
| | YFGSGSNYWGQGTLVTVSS | | |
| Mature light | DIQMTQSPSSLSASVGDRVTITCKTSQ | From U.S. Pat. No. 5,840,299 | 98 |
| chain variable | DINKYMAWYQQTPGKAPRLLIHYTSA | SEQ ID NO: 7 | |
| sequence La | LQPGIPSRFSGSGSGRDYTFTISSLQPQ | | |
| | DIATYYCLQYDNLWTFGQGTKVEIK | | |
| Mature light | DIQMTQSPSSLSASVGDRVTITCQASQ | From U.S. Pat. No. 5,840,299 | 99 |
| chain variable | DIIKYLNWYQQTPGKAPRLLIYEASNL | SEQ ID NO: 8 | |
| sequence Lb | QAGIPSRFSGSGSGRDYTFTISSLQPQD | | |
| | IATYYCQQYQSLPYTFGQGTKLQIT | | |

Natalizumab binds to the alpha4 integrin antigen, a subunit of Very Late Antigen 4 (VLA-4) protein complex (alpha4beta1 integrin) which mediates adhesion and migration of immune cells (e.g. Th-1 cells) through interaction with its ligand, vascular cell adhesion molecule (VCAM)-1 and mucosal addressin cellular adhesion molecule-1 (MAdCAM-1), on the endothelial cell surface. The alpha4beta4 integrins are expressed on the surface of most white leukocytes, including activated lymphocytes. Integrins are believed to play an important role in immune cell adhesion to the endothelial cell layer on blood vessels, facilitating their subsequent migration into inflamed tissues. Several studies implicate alpha4beta1 integrin heterodimers (VLA-4) in CNS inflammation (Yednock et al., Nature, 1992, 356, 63-66; Steffen et al., Am. J. Path. 145:189-201 (1994); Christensen et al., J. Immunol. 1995, 154, 5293-5301; the contents of each of which are herein incorporated by reference in their entirety.)

Natalizumab is an immunomodulator which functions by stopping activated inflammatory cells, including T-lymphocytes, of the immune system from reaching the brain (crossing the blood-brain barrier (BBB)) and spinal cord and causing damage. The binding of natalizumab therefore inhibits the alpha4-mediated adhesion of leukocytes to their counter-receptor(s), an early event in many inflammatory responses, particularly those of the central nervous system, such as multiple sclerosis (MS). The clinical effect of natalizumab in multiple sclerosis may be a secondary result of its blockade of the molecular interaction of alpha4beta1-integrin expressed by inflammatory cells with VCAM-1 on vascular endothelial cells, and with CS-1 and/or osteopontin expressed by parenchymal cells in the brain (Rice G P et al., anti-alpha4 integrin therapy for multiple sclerosis: mechanisms and rationale, Neurology, 2005, 64, 1336-1642; the content of which is herein incorporated by reference in its entirety.)

Natalizumab is being investigated for treating inflammatory and autoimmune diseases, such as multiple Sclerosis, Crohn's Disease and Rheumatoid Arthritis.

### Multiple Sclerosis (MS)

MS is a serious and disabling inflammatory and autoimmune disease of young adults, with a peak age of onset in the third decade of life. Most individuals present with the relapsing-remitting form of the disease and experience recurrent attacks, which, over time, result in accumulating permanent physical disability and cognitive decline. Almost 70% of patients will develop secondary progressive MS. Current treatments are minimally effective for secondary progressive MS.

Crohn's Disease is a debilitating disease that frequently causes diarrhea and abdominal cramps as well as fever, bleeding, and weight loss.

Natalizumab (Tysabri) is in clinical trial for its anti-tumor activity in patients with relapsed or refractory multiple myeloma. It could be also used in combination for the treatment of B-cell malignancies where it overcomes the resistance to rituximab (Mraz M et al., Bone marrow stromal cells protect lymphoma B-cells from rituximab-induced apoptosis and targeting integrin α-4-β-1 (VLA-4) with natalizumab can overcome this resistance, British J of Hematology, 2011, 155, 53-64; the content of which is herein incorporated by reference in its entirety.). Natalizumab may also be used to treat acute myelogenous leukemia (AML) as described in US patent publication NO: 20100266587; the content of which is herein incorporated by reference in its entirety.

Natalizumab is also being investigated for treating other autoimmune diseases, active ulcerative colitis, inflammatory bowel disease, rheumatoid arthritis, intestinal inflammation (Gordon F H et al., Aliment Pharmacol Ther. 2002, 16, 699-705; US patent publication NO: US20100303780; the contents of which are herein incorporated by reference in their entirety.)

It is expected that the polynucleotides of the present invention may be used to treat any of the diseases or disorders outlined above.

Cases have been reported that patients who had received natalizumab (Tysabri) treatment were diagnosed having progressive multifocal leukoencephalopathy (PML). PML is a rare infection of the central nervous system caused by a virus that can affect patients who have a compromised immune system, and usually causes death or severe disability. There is no known treatment, prevention, or cure for PML. Though the incidence of PML with natalizumab (Tysabri) remains unknown, the risk of PML when Tysabri is taken restricts its use in certain population of patients. Natalizumab is also restricted to patients who have compromised immune system (e.g. caused by HIV infection or AIDS, leukemia or lymphoma, or an organ transplant, or medicines that can weaken the immune system).

Other common side effects with natalizumab (Tysabri) include unusual or serious infections, allergic reactions during infusion or after receiving natalizumab (Tysabri), liver damage, and other side effects.

In some embodiments, the polynucleotides encoding natalizumab may be used together with other antibodies specific for alpha4-integrin or anti-VLA-4 antibodies, including, but not limited to, immunoglobulins described in U.S. Pat. No. 8,226,950 assigned to Biogen Idec; U.S. Pat. Nos. 8,246,958; 7,829,092; 6,602,503 and 6,551,593; and US patent publication No. 20020197233 by Relton et al; the contents of each of which are incorporated by reference in their entirety. Several additional VLA-4 binding monoclonal antibodies, such as HP2/1, HP2/4, L25 and P4C2, are described, e.g., in U.S. Pat. No. 6,602,503; Sanchez-Madrid et al., Eur. J. Immunol., 1986, 16, 1343-1349; Hemler et al., J. Biol. Chem. 1987, 2, 11478-11485; Issekutz and Wykretowicz, J. Immunol., 1991, 147: 109 (TA-2 mab); Pulido et al., J. Biol. Chem., 1991, 266(16): 10241-10245; and U.S. Pat. No. 5,888,507; the contents of each of which are incorporated by reference in their entirety.)

In some embodiments, the polynucleotides encoding natalizumab may be used to block the interaction between alph4-intergrin/VLA-4 and its ligand VCAM-1, therefore stopping recruiting active leukocytes in many disease conditions (e.g. MS, CD and multiple myeloma). Alpha 4 β1 integrin is a cell-surface receptor for VCAM-1, fibronectin and possibly other molecules. the polynucleotides encoding natalizumab may be used together with other antagonists capable of binding to any integrin containing an alpha4 subunit such as VLA-4 on the surface of VLA-4 bearing cells and/or alpha4beta7 integrin on the surface of alpha4beta7-bearing cells and/or to their respective α4 ligands such as VCAM-1 and MadCAM, respectively, or on the surface of VCAM-1 and MadCAM bearing cells.

In some embodiments, the polynucleotides encoding natalizumab may be used as an immunomodulatory agent, alone or in combination with other immunomodulatory or immunosuppressive agents. Said agents may be selected from the group consisting of epratuzumab, adalimumab, rituximab, alemtuzumab, basiliximab, efalizumab, infliximab, muromomab veltuzumab, milatuzumab, daclizumab, basiliximab, tacrolimus, sirolimus, mycophenolate (as sodium or mofetil), Cyclosporine A, Glucocorticoids, Anti-CD3 monoclonal antibodies (OKT3), Antithymocyte globulin (ATG), Anti-CD52 monoclonal antibodies (campath 1-H), Azathioprine, Everolimus, Dactinomycin, Cyclophosphamide, Platinum, Nitrosurea, Methotrexate, Azathioprine, Mercaptopurine, Muromonab, IFN gamma, Infliximab, Etanercept BG12, and fingolimod (see, e.g. (Guagnozzi D and Caprilli R, Natalizumab in the treatment of Crohn's disease, Biologics, 2008, 2, 275-284.]

In some embodiments, the polynucleotides encoding natalizumab may be used to treat inflammatory and autoimmune diseases, alone or in combination with other agents for treating inflammatory and autoimmune diseases, see e.g. U.S. Pat. No. 8,518,406 (antibody against MCP-1 N1pE for inflammatory diseases); US patent publication Nos. 20070207141; 20100196318; PCT patent publication Nos. WO2007100770; the contents of each of which are incorporated by reference in their entirety.

In some embodiments, the polynucleotides encoding natalizumab may be used to treat multiple sclerosis, such as relapsing-remitting multiple sclerosis, secondary progressive multiple sclerosis, primary progressive multiple sclerosis, or clinically isolated syndrome. In particular, MS is relapsing forms of MS (see, e.g. US patent publication NOs: US20120276048; 20070207141; US20100021429; the contents of each of which are incorporated by reference in their entirety.)

For example, the polynucleotides encoding natalizumab may be used in combination with other agents such as a steroid (e.g. glucocorticoid), an anti-inflammatory compound, an immunosuppressive compound, and an antioxidant to treat MS (see, e.g. U.S. Pat. No. 8,394,763 (PPIase inhibitors for MS); U.S. Pat. No. 8,344,153 PI3K inhibitors for MS; the contents of each of which are incorporated by reference in their entirety.)

In some embodiments, the polynucleotides encoding natalixumab may be used to treat Crohn's Disease (CD).

According to the present invention, the natalizumab polynucleotides of the present invention are engineered such that they may comprise one or more coding regions.

The coding regions of the natalizumab polynucleotides may encode any of the regions or portions of the natalizumab antibody. They may also further comprise coding regions not found in the original or parent natalizumab antibody.

The natalizumab polynucleotides of the present invention may be engineered to produce any of the 5 standard classes of immunoglobulins as shown in FIG. 1 using the natalizumab antibody or any of its component parts as a starting molecule.

The natalizumab polynucleotides may also be engineered according to the present invention to produce a variant natalizumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Ofatumumab Parent Molecule or Antibody

According to the present invention, ofatumumab polynucleotides or constructs and their associated ofatumumab compositions are designed to produce the ofatumumab antibody, a variant or a portion thereof in vivo.

Ofatumumab (synonym: 2F2; HuMax-CD20) is commercially available from GlaxoSmithKline and Genmab under the trade name ARZERRA.

Ofatumumab is the first human monoclonal antibody approved by the Food and Drug Administration for treating chronic lymphocytic leukemia (CLL) refractory to fludarabine and alemtuzumab. Ofatumumab may be used in combination with chlorambucil for the treatment of chronic lymphocytic leukemia (CLL) patients who have not received prior treatment and are inappropriate for fludarabine-based therapy. Ofatumumab is also being investigated clinically for treating other B-cell malignancies (e.g. follicular lymphoma diffuse large B-cell non-hodgkin's lymphoma), autoimmune diseases (e.g. rheumatoid arthritis) and infections (Jagloski S M et al., blood, 2010, 116, 3705-3714; Cang et al., J Hemat. Onco., 2012, 5, 64; Teeling et al., Blood, 2004, 104, 1793-1800; Zhang, mAbs, 2009, 4, 326-331; Kurrasch R et al., J Rheumatol. 2013, 40, 1089-1096; Rosman et al., BMC Medcine, 2013, 11, 88; the contents of each of which are herein incorporated by reference in their entirety).

In some patients, this therapeutic antibody causes serious and sometimes life-threatening side effects, such as neutropenia, pneumonia and other respiratory infections. In addition, the administration of antibody based therapy raises a potential for immunogenicity to these therapeutic proteins in patients. In other words, the patient may develop serum antibodies against the therapeutic proteins.

Ofatumumab is an anti-CD20 IgG1κ human monoclonal antibody with a molecular weight of approximately 149 kDa, which binds an epitope on the CD20 antigen encompassing parts of the small and large extracellular loop (Teeling et al., J Immunology, 2006, 177, 362-371; Lin T, Pharmacogemonics and personalized medicine, 2013, 3, 51-59; the content of each of which are herein incorporated by reference in their entirety). The antibody was generated via transgenic mouse and hybridoma technology and is produced in a recombinant murine cell line (NS0) using standard mammalian cell cultivation and purification technologies.

Ofatumumab is first described as 2F2 antibody in PCT patent publication WO2004035607, (also in PCT patent publication NO: WO2005103081; US patent publication NO: 20040167319; U.S. Pat. No. 8,529,902, the contents of each of which are herein incorporated by reference in their entirety). Sequences of human IgG heavy chain variable region and human kappa light chain variable region of ofatumumab (2F2) and the nucleic acids that encode such amino acids are listed in Table 21. The sequences of three CDR domains of heavy and light chains of ofatumumab (2F2) are also listed in Table 21.

Ofatumumab is clinically tested for the treatment of non-Hodgkin's lymphoma (NHL), chronic lymphocytic leukemia (CLL), and rheumatoid arthritis (RA). See Teeling et al., Blood, 2004, 104, 1793; and Teeling et al., J. Immunology, 2007, 177, 362-371; Jagloski S M et al., blood, 2010, 116, 3705-3714; Cang et al., J Hemat. Onco., 2012, 5, 64; the contents of each of which are herein incorporated by reference in their entirety. Ofatumumab is also being studied for treating lymphoma, rheumatoid arthritis and multiple sclerosis. In particular, this antibody has been used (considered by the US Food and Drug Administration and the European Medicines Agency for marketing approval) as a treatment for chronic lymphocytic leukemia refractory to fludarabine and alemtuzumab in 2009.

Certain sequences encoding ofatumumab fragments, domains or heavy or light chains are given in Table 21. The table is not an exhaustive list and any fragment or portion of the sequence may be encoded in the ofatumumab polynucleotides of the invention.

TABLE 21
Table of Ofatumumab Sequences

| | | | SEQ |
|---|---|---|---|
| | | | ID |
| Description | Sequence | Source | NO. |
| Human | MELGLSWIFLLAILKGVQCEVQLVESGG | From | 100 |
| Heavy chain | GLVQPGRSLRLSCAASGFTFNDYAMHW | U.S. Pat. No. 8,529,902, SEQ | |
| variable | VRQAPGKGLEWVSTISWNSGSIGYADSV | ID NO: 2 | |
| region | KGRFTISRDNAKKSLYLQMNSLRAEDTA | | |
| | LYYCAKDIQYGNYYYGMDVWGQGTTV | | |
| | TVSS | | |
| Nucleic acids | ATGGAGTTGGGACTGAGCTGGATTTT | From | 101 |
| that encode | CCTTTTGGCTATTTTAAAAGGTGTCCA | U.S. Pat. No. 8,529,902, SEQ | |
| human IgG | GTGTGAGTGCAGCTGGTGGAGTCTGG | ID NO: 1 | |
| heavy chain | GGGAGGCTTGGTACAGCCTGGCAGGT | | |
| variable | CCCTGAGACTCTCCTGTGCAGCCTCT | | |
| region | GGATTCACCTTTAATGATTATGCCAT | | |
| | GCACTGGGTCCGGCAAGCTCCAGGGA | | |
| | AGGGCCTGGAGTGGGTCTCAACTATT | | |
| | AGTTGGAATAGTGGTTCCATAGGCTA | | |
| | TGCGGACTCTGTGAAGGGCCGATTCA | | |
| | CCATCTCCAGAGACAACGCCAAGAAG | | |
| | TCCCTGTATCTGCAAATGAACAGTCT | | |
| | GAGAGCTGAGGACACGGCCTTGTATT | | |
| | ACTGTGCAAAAGATATACAGTACGGC | | |
| | AACTACTACTACGGTATGGACGTCTG | | |
| | GGGCCAAGGGACCACGGTCACCGTCT | | |
| | CCTCAG | | |
| Human kappa | MEAPAQLLFLLLLWLPDTTGEIVLTQSPA | From | 102 |
| Light chain | TLSLSPGERATLSCRASQSVSSYLAWYQQ | U.S. Pat. No. 8,529,902, SEQ | |
| variable | KPGQAPRLLIYDASNRATGIPARFSGSGS | ID NO: 4 | |
| region | GTDFTLTISSLEPEDFAVYYCQQRSNWPIT | | |
| | FGQGTRLEIK | | |
| Nucleic acids | ATGGAAGCCCCAGCTCAGCTTCTCTT | From | 103 |
| that encode | CCTCCTGCTACTCTGGCTCCCAGATA | U.S. Pat. No. 8,529,902, SEQ | |
| human kappa | CCACCGGAGAAATTGTGTTGACACA | ID NO: 3 | |
| light chain | GTCTCCAGCCACCCTGTCTTTGTCTC | | |
| variable | CAGGGGAAAGAGCCACCCTCTCCTG | | |
| region | CAGGGCCAGTCAGAGTGTTAGCAGC | | |
| | TACTTAGCCTGGTACCAACAGAAAC | | |
| | CTGGCCAGGCTCCCAGGCTCCTCATC | | |
| | TATGATGCATCCAACAGGGCCACTG | | |
| | GCATCCCAGCCAGGTTCAGTGGCAG | | |
| | TGGGTCTGGGACAGACTTCACTCTCA | | |
| | CCATCAGCAGCCTAGAGCCTGAAGA | | |
| | TTTTGCAGTTTATTACTGTCAGCAGC | | |
| | GTAGCAACTGGCCGATCACCTTCGGC | | |
| | CAAGGGACACGACTGGAGATTAAAC | | |
| Heavy chain | NDYAMH | From | 104 |
| CDR1 | | U.S. Pat. No. 8,529,902, SEQ | |
| | | ID NO: 13 | |
| Heavy chain | TISWNSGSIGYADSVKG | From | 105 |
| CDR2 | | U.S. Pat. No. 8,529,902, SEQ | |
| | | ID NO: 14 | |
| Heavy chain | DIQYGNYYYGMDV | From | 106 |
| CDR3 | | U.S. Pat. No. 8,529,902, SEQ | |
| | | ID NO: 15 | |
| Light chain | RASQSVSSYLA | From | 107 |
| CDR1 | | U.S. Pat. No. 8,529,902, SEQ | |
| | | ID NO: 16 | |
| Light chain | DASNRAT | From | 108 |
| CDR2 | | U.S. Pat. No. 8,529,902, SEQ | |
| | | ID NO: 17 | |
| Light chain | QQRSNWPIT | From | 109 |
| CDR3 | | U.S. Pat. No. 8,529,902, SEQ | |
| | | ID NO: 18 | |

Ofatumumab binds specifically to both the small and large extracellular loops of the CD20 antigen (see, e.g. Teeling et al., J Immunology, 2006, 177, 362-371; Lin T, Pharmacogemonics and personalized medicine, 2013, 3, 51-59; the content of each of which are herein incorporated by reference in their entirety). The CD20 molecule is expressed on normal B lymphocytes (pre-B- to mature B-lymphocyte) and on B-cell CLL. CD20 regulates an early step(s) in the activation process for cell cycle initiation and differentiation, and possibly functions as a calcium ion channel. The CD20 molecule is not shed from the cell surface and is not internalized following antibody binding. Studies show that the Fab domain of ofatumumab binds to the CD20 molecule and the Fc domain mediates immune effector functions to result in B-cell lysis in vitro. Data suggest that possible mechanisms of cell lysis include complement-dependent cytotoxicity (CDC) and antibody-dependent, cell-mediated cytotoxicity (ADCC).

CLL is the most common form of leukemia in adults. Based on estimates by the American Cancer Society, CLL will account for more than 15,680 new cases and more than 4,580 deaths in the United States of America alone in 2013. At present, no curative chemotherapy is available.

Ofatumumab is the first human monoclonal antibody approved by the US Food and Drug Administration for treating chronic lymphocytic leukemia refractory to fludarabine and alemtuzumab. Ofatumumab may be used in combination with chlorambucil for the treatment of patients with chronic lymphocytic leukemia (CLL) who have not received prior treatment and are inappropriate for fludarabine-based therapy. The ofatumumab polynucleotides of the present invention may be used for the same disorder and/or condition.

Non-Hodgkin's lymphoma (NEIL) refers to any of a large group of cancers of lymphocytes (white blood cells). Non-Hodgkin lymphomas can occur at any age and are often marked by lymph nodes that are larger than normal, fever, and weight loss. Non-Hodgkin's lymphoma (NHL) may include mantle cell lymphoma, diffuse large B-cell lymphoma (DLBCL), chronic lymphocytic leukemia (CLL)/small lymphocytic lymphoma (SLL), or follicular lymphoma (FL). Follicular lymphoma (FL) is a subgroup of Non Hodgkin's Lymphomas (NHL) and is the second most common lymphoma in the US and Europe, accounting for 11 to 35% of all NHL. Diffuse Large B-Cell Lymphoma (DLBCL) is a cancer of the B-lymphocytes and represents 30% of non-Hogdkin's lymphomas in adults and is the most common lymphoid malignancy in the western world. There are an estimated 63,000 new cases of DLBCL diagnosed in the US per year. The median age at diagnosis is about 65 years. Waldenstrom's Macroglobulinemia (WM) is a type of slow-growing non-Hodgkin's lymphoma, characterized by the infiltration of the bone marrow with lymphoplasmacytic cells and the detection of an IgM monoclonal gammopathy in the serum.

Clinical studies are examining the efficacy and safety of ofatumumab in treating FL, DLBCL and WM, specially the relapsed or refractory Non-Hodgkin's lymphoma (NHL) (Hagenbeek et al., Blood, 2008, 111, 5486-5495; Coiffier B et al., Br. J Hematol. 2013, 163, 334-342; Issa et al., Clin Investig, 2011, 1, 815-824; the contents of each of which are herein incorporated by reference in their entirety). The ofatumumab polynucleotides of the present invention may be used for the same disorder and/or condition.

Pemphigus Vulgaris is a rare, chronic skin disorder in which the immune system malfunctions and produces antibodies that attack healthy cells in the skin and mucous membranes. This causes burn-like blisters and sores to appear on the skin or mucous membranes and may lead to secondary skin infections, dehydration, spread of infection through the bloodstream (sepsis) and death. The incidence of pemphigus vulgaris is approximately seven people per million worldwide. The current standard treatment for pemphigus vulgaris is systemic glucocorticoids. Ofatumumab may be used, in combination with other therapeutic agents, such as anti-CD22 antibodies, anti-CD47 antibodies, to treat Pemphigus Vulgaris, see, e.g. US patent publication NOs: US20110020328; 20130142787; PCT patent publication NOs: WO2012024223; WO2013085893; the content of each of which are herein incorporated by reference in their entirety. The ofatumumab polynucleotides of the present invention may be used for the same disorder and/or condition.

Multiple Sclerosis (MS) is an inflammatory disease of the central nervous system. MS is twice as common in females as in males, occurs with a peak incidence at the age of 35 years and incidence varies widely in different populations and ethnic groups. The most common form of MS is relapsing remitting MS (RRMS) characterized by unpredictable recurrent attacks where the symptoms usually evolve over days and are followed by either complete, partial or no neurological recovery. No progression of neurological impairment is experienced between attacks. The recently successful targeting of B cells in patients with multiple sclerosis (MS) using anti-CD20 monoclonal antibodies (mAbs) has established B cells contribution to MS disease activity. The therapeutic efficacy of B cell depletion in MS from clinical trials of different anti-CD20 mAbs in patients with MS demonstrates the therapeutic potentials of targeting the CD20 surface antigen. Ofatumumab is investigated in phase II clinical trials for treating relapsing-remitting MS (RRMS), and in progressive forms of MS (Rommer P et a., Clin. Exp. Immunol, 2013, 10, e12197; Nocholas et al., J Cent. Nerv. Syst. Dis, 2012, 4, 81-103; the contents of each of which are herein incorporated by reference in their entirety). The ofatumumab polynucleotides of the present invention may be used for the same disorder and/or condition.

In some embodiments, the polynucleotides encoding ofatumumab may be used in combination with other anti-CD-20 antibodies. Such anti-CD 20 antibodies include, but are not limited to, TRU-015, obinutuzumab (GA101), SBI-087, hA20, 11B8, 7D8, 2C6 IgG1 (as disclosed in WO2004056312), ocaratuzumab (AME-133), DXL 625, TRU-015, IMMU-106, DXL625, ocrelizumab (2H7.v16, PRO-70769, R-1594), Bexxar® (tositumomab), Rituxan®/MabThera® (Rituximab), veltuzumab, anti-CD20 antibodies as described in U.S. Pat. Nos. 8,465,741; 8,097,713; 8,057,793; 7,879,984; and US patent publication NOs: US20130195846; US20090136516; US20090130089; the contents of each of which are herein incorporated by reference in their entirety.

In some embodiments, the polynucleotides encoding ofatumumab may be used treat any cancer (tumor) expressing CD20 (i.e. B-cell malignancies or CD20 positive cancer), including, precursor B- and T-cell neoplasms, mature B-cell neoplasms, Hodgkin's lymphoma, and immunodeficiency associated lymphoproliferative disorders, CLL and NHL, alone or in combination with other anti-cancer treatments (see, e.g. US patent publication NOs: 20110274697 and 20110189175; Cang et al., J Hematol Oncol. 2012, 5, 64; Jaglowski et al., Blood, 2010, 116, 3705-3714; the contents of each of which are herein incorporated by reference in their entirety). As non-limiting examples, the polynucleotides encoding ofatumumab may be used with bendamustine, bortezomib, CAL-101, chlorambucil, cyclophosphamide, dexamethasone, docetaxel, doxorubicin, endostatineverolimus, etoposide, fludarabine, fostamatinib, hydroxydaunorubicin, ibritumomab, ifosphamide, lenalidomide, mesalazine, paclitaxel, pentostatin, prednisone, temsirolimus, thalidomide, tositumomab, vincristine, bruton's tyrosine kinase (BtK) inhibitors as described in US Patent publication NO: 20130273030, an immunomodulator as described in PCT patent publication NO: WO2013129936; the contents of each of which are herein incorporated by reference in their entirety.

In some embodiments, the polynucleotides encoding ofatumumab may be used as a B-cell depleting agent, alone or in combination with other B-cell depleting agents, to reduce the number of B-cells or treating a disease or disorder associated with aberrant B-cell activity in a subject having or suspected having the disease or disorder. Said other B-cell depleting agents may include, but are not limited to, a B-cell depleting anti-CD20 antibody or CD20-binding antibody fragment thereof (e.g. Rituximab, Ocrelizumab, GA101, BCX-301, Veltuzumab and DXL 625, TRU-015, SBI-087); Methotrexate; CD37 specific binding molecules (e.g. anti-CD37antibodies, SMIP protein; see, e.g. US patent publication NO: 20100135900; the content of which is herein incorporated by reference in its entirety). For example, such B cell depleting agents may be used to treat chronic fatigue syndrome and optionally myalgic encephalomyelitis (see, e.g. U.S. Pat. No. 7,914,785; the content of which is herein incorporated by reference in its entirety).

In some embodiments, the polynucleotides encoding ofatumumab may be used as a general immunotherapy antibody, for treating cancer in combination with other anti-cancer regimens. Such anti-cancer agents may include, but are not limited to, alemtuzumab, trastuzumab, gemtuzumab, gemtuzumab-ozogamicin, cetuximab, bevacizumab, zalutumumab, lintuzumab, lumiliximab, epratuzumab and pertuzumab, rituximab; antibodies against tissue factor, killer Ig-like receptors (KIR), laminin-5, EGF receptor, VEGF receptor, PDGF receptor, HER-2/neu receptor, prostate-specific antigen (PSA), prostate stem cell antigen (PSCA), carcinoembryonic antigen (CEA), cancer antigen 125 (CA125), tumor-associated calcium signal transducer antigen (KSA), CTLA-4, leukocyte immunoglobulin-like receptor 1 (LIR), CD94, and NKG2A, CD38, CD200, VEGF and IGF 1R (see, e.g. US patent publication NOs: 20110262525; 20110129456; 20110014117; US20110097340; US20130209355; US20130189258; the content of each of which are incorporated herein by reference in their entirety).

In one aspect, the polynucleotides encoding ofatumumab as an antibody based therapy may be used in combination with other chemotherapy regimens, such as everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a BcI-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380.

In some embodiments, the polynucleotides encoding ofatumumab may be used to treat Chronic Obstructive Pulmonary Disease (COPD) as described in PCT patent publication NO: WO2008003319; the content of which is herein incorporated by reference in its entirety).

In some embodiments, the polynucleotides encoding ofatumumab may be used to treat progressive multiple sclerosis (e.g. relapsing remitting multiple sclerosis), either alone or in combination with other agents such as a second anti-CD20 antibody or the polynucleotide encoding said second anti-CD20 antibody (e.g. TRU-015 or SBI-087, GA101, hA20, Rituximab) (see, e.g. US patent publication NOs: 20120225070 and 20100158903; the contents of each of which are herein incorporated by reference in their entirety); other FDA approved disease modifying therapies such as IFN-β-1a (Avonex, Rebif), IFN-β-1b (Betaseron, Extavia), glatiramer acetate (Copaxone), mitoxantrone (Novantrone), natalizumab (Tysabri) and fingolimod (Gilenya); other anti-MS therapeutic monoclonal antibodies such as alemtuzumab, daclizumab, Ocrelizumab and ofatumumab; or oral agents (e.g. BG12, laquinimod, and teriflunomide) (Nicholas et al., J Cent. Nerv. Syst. Dis, 2012, 4, 81-103; the content of which is herein incorporated by reference in its entirety).

In some embodiments, the polynucleotides encoding ofatumumab may be used to treat autoimmune diseases, for example, rituximab refractory rheumatoid arthritis in combination with Veltuzumab, another anti-CD20 antibody; systemic lupus erythematosus (SLE) with B-lys antagonist and/or other anti-CD20 antibodies (e.g. RITUXAN®, ocrelizumab, TRU-015, and DXL625); inflammatory bowel disease (IBD) (e.g. ulcerative colitis (UC), Crohn's disease) (see, e.g. US patent publication NOs: US20090169550; US20060233797; and US20090148442; the contents of each of which are herein incorporated by reference in their entirety.

According to the present invention, the ofatumumab polynucleotides of the present invention are engineered such that they may comprise one or more coding regions.

The coding regions of the ofatumumab polynucleotides may encode any of the regions or portions of the ofatumumab antibody. They may also further comprise coding regions not found in the original or parent ofatumumab antibody.

The ofatumumab polynucleotides of the present invention may be engineered to produce any of the 5 standard classes of immunoglobulins as shown in FIG. 1 using the ofatumumab antibody or any of its component parts as a starting molecule.

The ofatumumab polynucleotides may also be engineered according to the present invention to produce a variant ofatumumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Omalizumab Parent Molecule or Antibody

According to the present invention, omalizumab polynucleotides or constructs and their associated omalizumab compositions are designed to produce the omalizumab antibody, a variant or a portion thereof in vivo.

Omalizumab (synonyms: anti-IgE monoclonal antibody E25; E25; humanized anti-IgE mAb; IGE 025; olizumab; RhuMa-E25) is marketed under the trade name Xolair by Roche/Genentech and Novartis.

Omalizumab is a humanized antibody IgG1 that selectively binds to human immunoglobulin E (IgE). The primary use of omalizumab is for treating severe, persistent allergic asthma that does not respond to high dose of corticosteroids. Omalizumab (Xolair) has received approval by the Food and Drug Administration and in many other regions/countries (e.g. the Europe Union) for treating patients 12 years and older with moderate to severe allergic asthma. Clinical studies show that the efficacy of omalizumab is evident for severe asthmatics and the response rates among treated severe “allergic” asthma patients are 60-80% or higher. The primary benefits for the responding patients are reduced numbers of exacerbations, improved lung function, reduced numbers of emergency visits to the doctors, reduced days of hospitalization, and increased quality of life measurements. The other major benefit is that most responding patients can reduce or spare entirely the use of corticosteroids, which cause multiple serious side effects, when used at high doses for extended periods.

However, Omalizumab's cost is high, ranging from $500 to $2,000 a month or $6,000 to $24,000 a year, limiting its availability in developing countries and leading to rationing in countries with a general healthcare system such as the UK. In addition, anaphylaxis, a severe allergic disease happens in some patients who receive omalizumab treatment.

Omalizumab is a recombinant DNA-derived humanized IgG1 kappa monoclonal antibody that selectively binds to human immunoglobulin E (IgE). Omalizumab (Xolair) is produced by a Chinese hamster ovary cell suspension culture in a nutrient medium containing the antibiotic gentamicin.

Omalizumab was originally isolated from human-mouse monoclonal E25 clone pSVIE26 gamma-chain) (see, e.g. U.S. Pat. No. 5,994,511, and PCT patent publication NO: WO1999001556, by Lowman et al, assigned to Genentech, Inc.; the contents of each of which are herein incorporated by reference in their entirety.) The original mouse anti-IgE clone (MaE11) was generated by immunizing mice with purified human IgE molecules. This murine antibody clone directed against human IgE (MaE11) was used to provide the CDR regions which were substituted into an IgG1 immunoglobulin framework (rhuMaE25), through DNA recombinant techniques (U.S. Pat. No. 5,994,511, in FIG. 12, and in the sequences ID-No. 13-14, the contents of which are incorporated herein by reference in their entirety), Residues were further modified by site mutagenesis, therefore, improve the affinity for IgE. The amino acid sequences of both light and heavy chains of Omalizumab are listed in Table 22. As one ordinarily skilled in the art would expect, all variants and derivatives are included in the present invention.

Certain sequences encoding omalizumab fragments, domains or heavy or light chains are given in Table 22. The table is not an exhaustive list and any fragment or portion of the sequence may be encoded in the omalizumab polynucleotides of the invention.

TABLE 22
Table of Omalizumab Sequences

| | | SEQ ID |
|---|---|---|
| Description | Sequence | NO. |
| Anti-IgE | EVQLVESGGGLVQPGGSLRLSCAVSGYSITSGYSWNW | 110 |
| antibody VH | IRQAPGKGLEWVASITYDGSTNYADSVKGRFTISRDDS | |
| domain chain 1 | KNTFYLQMNSLRAEDTAVYYCARGSHYFGHWHFAV | |
| | WGQGTLVTVSSGPSVFPLAPSSKSTSGGTAALGCLVK | |
| | DYFPEPVTVSWNSGALTSGVHTFPAVLQSSGLYSLSSV | |
| | VTVPSSSLGTQTYICNVNHKPSNTKVDKKAEPKSCDK | |
| | THTCPPCPAPELLGGPSVFLFPPKPKDTLMISRTPEVTC | |
| | VVVDVSHEDPEVKFNWYVDGVEVHNAKTKPREEQY | |
| | NSTYRVVSVLTVLHQDWLNGKEYKCKVSNKALPAPI | |
| | EKTISKAKGQPREPQVYTLPPSRDELTKNQVSLTCLVK | |
| | GFYPSDIAVEWESNGQPENNYKTTPPVLDSDGSFFLYS | |
| | KLTVDKSRWQQGNVFSCSVMHEALHNHYTQKSLSLS | |
| | PGK | |
| Anti-IgE | DIQLTQSPSSLSASVGDRVTITCRASQSVDYDGDSYMN | 111 |
| antibody VL | WYQQKPGKAPKLLIYAASYLESGVPSRFSGSGSGTDF | |
| domain chain 1 | TLTISSLQPEDFATYYCQQSHEDPYTFGQGTKVEIKRT | |
| | VAAPSVFIFPPSDEQLKSGTASVVCLLNNFYPREAKVQ | |
| | WKVDNALQSGNSQESVTEQDSKDSTYSLSSTLTLSKA | |
| | DYEKHKVYACEVTHQGLSSPVTKSFNR | |

Omalizumab selectively binds to free human immunoglobulin E (IgE) in the blood and interstitial fluid and to membrane-bound form of IgE (mIgE) on the surface of mIgE-expressing B lymphocytes. Omalizumab specifically binds to the cepsilon 3 domain of human IgE Fc region, which is the site of high-affinity IgE receptor binding. Therefore, omalizumab does not bind to IgE that is already bound by the high affinity IgE receptor (FcεRI) on the surface of mast cells, basophils and antigen-presenting dendritic cells (Chang et al., Anti-IgE antibodies for the treatment of IgE-mediated allergic diseases, Adv Immunol. 2007, 93, 63-119, the content of which is herein incorporated by reference in its entirety.)

Immunoglobulin E is a member of the immunoglobulin family that is secreted by, and expressed on the surface of B-cells or B-lymphocytes. IgE mainly functions as an immune defender against parasites. IgE also plays an essential role in type I hypersensitivity, which manifests various allergic diseases, such as allergic asthma, allergic rhinitis, food allergy, and some types of chronic urticaria and atopic dermatitis. In addition, IgE plays a pivotal role in allergic conditions, such as anaphylactic reactions to certain drugs, bee stings, and antigen preparations used in specific desensitization immunotherapy. The IgE molecules (e.g. allergen-specific and allergen-nonspecific IgE), bind to the high affinity IgE receptor (FcεRI) on the surface of mast cells and basophils. Under certain conditions, including but not limited to when the allergenic substances are taken in by a sensitized individual at substantial amounts, the allergenic proteins bind to the allergen-specific IgE bound by FcεRI on the surface of mast cells and basophils and trigger the activation of those inflammatory cells, which release a host of inflammatory mediators, such as histamine, leukotrienes, tryptase, inflammatory cytokines, and other factors, causing various allergic symptom/diseases. In addition, IgE binds to B-cells (as well as to monocytes, eosinophils and platelets) through its Fe region to a low affinity IgE receptor, known as FcεRII.

As discussed above, omalizumab's binding to IgE can neutralize circulating IgE by preventing IgE from binding to its high-affinity mast-cell receptor, therefore blocking IgE signaling. Possible mechanisms may include 1) inhibiting Erk1/2 MAPK to stimulate airway (smooth muscle) cell proliferation and remodeling; 2) decreasing/inhibiting the release of inflammatory histamine and other mediators (e.g. chemokine CCL15); 3) inducing eosinophil apoptosis (see, e.g. U.S. Pat. No. 6,290,957; Roth et al., Plos One, 2013, 8, e56015; Rup and Kahn, international Archives Allergy and Applied Immunology, 1989, 89, 387-393; the contents of each of which is herein incorporated by reference in their entirety.) The immune complexes formed between IgE and omalizumab in vivo are relatively small (molecular weight<1 million) and are therefore unlikely to cause organ damage.

Omalizumab function as an anti-IgE antagonist, blocking IgE mediated allergic reactions and other clinical conditions.

Allergic asthma (also called extrinsic asthma) is airway obstruction and inflammation that is partially reversible with medication. It has been estimated that as high as 20 to 40% of the populations who live in economically advanced countries are affected by allergy and seek medical help. According to Asthma and Allergy Foundation of America, Allergic asthma is the most common form of asthma in USA, affecting over 50% of the 20 million asthma sufferers. Over 2.5 million children under age 18 suffer from allergic asthma. Many of the symptoms of allergic and non-allergic asthma are the same (coughing, wheezing, shortness of breath or rapid breathing, and chest tightness). However, allergic asthma is triggered by inhaled allergens such as dust mite allergen, pet dander, pollen, mold, etc. resulting in asthma symptoms.

Immunoglobulin E (IgE) is the antibody in the body that plays a major role in allergic diseases. The body produces the IgE antibody when it detects an allergen and causes the “allergic cascade” to begin. While allergy occurs more frequently in individuals with higher serum IgE levels, such a correlation is only statistical and not absolute. Some allergic individuals have very low serum IgE, and some people with very high IgE have no allergic problems.

Anti-IgE antibodies have been an attractive strategy for treating allergic diseases such as asthma. (see, Thomson and Chauhuri, Clin Med Insights Circ Respir Pulm Med., 2012, 6, 27-40; the content of which is incorporated herein by reference in its entirety.) omalizumab treatment in patients with severe, persistent allergic asthma can reduce eosinophil numbers in the airway mucosa and the IgE bearing cells. Treatment of allergic subjects with omalizumab also reduces the release of Th2 cytokines from blood basophils.

The polynucleotides of the present invention may be used to treat the same or similar indications as the commercial antibody, omalizumab.

Omalizumab is also under many clinical studies for potential treatment of allergic asthma, perennial and seasonal allergic rhinitis, food allergy (e.g. peanut), occupational allergy, pollen allergy, latex allergy, atopic dermatitis, chronic idiopathic urticaria (i.e. chronic spontaneous urticaria), mastocytosis (e.g. cutaneous), eosinopholic gastroenteritis, nasal polyposis and other allergic disorders (Maurer and Hsieh, N Engl J Med, 2013, 368, 2530; Washish and Casale, Expert Opin Bio Ther, 2013, 13, 933-945; Khoriaty and Umetsu, Allergy Asthma Immunol Res., 2013, 5, 3-15; Thaiwat and Sangasapaviliya, Asian Pac J Allergy Immunol., 2011, 29, 357-360; the contents of each of which are herein incorporated by reference in their entirety.)

Omalizumab has also been studied in combination with allergen-based specific immunotherapy (allergy shot) for the purpose of reducing anaphylactic reactions when receiving allergen immunizations and of accelerating immunization schedule and dosing, so as to achieve therapeutic effects in shorter treatment periods and in broader patient populations.

Like the other protein and antibody drugs, the main adverse effect of omalizumab is anaphylaxis (a life-threatening systemic allergic reaction). The signs and symptoms of anaphylaxis include wheezing, shortness of breath, cough, low blood pressure, hives, swelling of the throat, etc, which may happen right after Xolair injection or hours later. This allergic reaction is not due to the binding of omalizumab to IgE, but because of the protein nature of the antibody drugs.

It is expected that the polynucleotides of the present invention will present a profile lacking the side effects and/or adverse reactions seen with the commercial antibody.

In some embodiments, the polynucleotides of the present invention may be used for treating a IgE mediated disease and clinical condition, including but not limited to, asthma (e g. allergic asthma and non-allergic asthma), allergic rhinitis, conjunctivitis (hay fever), eczema, urticaria (e.g. chronic spontaneous urticarial), atopic dermatitis, food allergies (e.g. peanut) and other allergic disease (e.g. occupational allergy and pollen allergy).

In one aspect, the polynucleotides encoding omalizumab may be used to treat allergic asthma in combination with other immunotherapeutic agents as described in US Patent application publication NOs: US 20040197326, US20070020256; US20080206237; US20090087426; US20110311520; the contents of each of which are herein incorporated by reference in their entirety.) The polynucleotide of the present invention may be used to treat other allergic disease such allergic rhinitis as described in PCT patent publication NO WO1997033616; the content of which is herein incorporated by reference in its entirety.

In one aspect, the polynucleotides encoding omalizumab may be used in combination with other anti-IgE antibodies for decreasing/inhibiting IgE signaling in a subject. Said other anti-IgE antibodies may include, but are not limited to, E26 and E27 as described in U.S. Pat. Nos. 5,994,511; 6,290,957; 6,682,735; 6,761,889; assigned to Genentech, Inc. and PCT patent publication Nos. WO1993004173; and WO1999001556); the anti-IgE antibody that binds to the extracellular domain of IgE on B cells but not basophils or in the secreted, or soluble form of IgE as described in U.S. Pat. Nos. 5,252,467 and 5,231,026; the anti-IgE antibody that prevents the binding of free IgE to FcεRI but does not bind to FcεRI-bound IgE as described by Presta et al (J Immunol, 1993, 151, 2623-2632); the anti-IgE antibodies as a therapy for allergy that bind to IgE on B cells, but not IgE on basophils disclose as described in U.S. Pat. No. 5,428,133; monoclonal antibodies that react with free IgE and thereby inhibit IgE binding to mast cells, and react with IgE when it is bound to the B-cell membrane, but do not bind with IgE when it is bound to the mast cell Fee receptor, nor block the binding, of IgE to the B-cell receptor as described in U.S. Pat. No. 4,940,782; and other anti-IgE antibodies as described in U.S. Pat. Nos. 5,449,760; 5,958,708; 6,066,718; 6,072,035; the contents of each of which are herein incorporated by reference in their entirety. According to the present invention, these anti-IgE antibodies may also be generated by the polynucleotides as described herein.

In some embodiments, the polynucleotides of the present invention may be used to inhibit IgE signaling, in combination with other IgE signal antagonists that can inhibit the biological activity of IgE, for example, anti-IgE antibodies and variants as described herein; IgE binding factor and fragments (e.g. U.S. Pat. No. 4,946,788; the content of which is herein incorporated by reference in its entirety,) small molecules such as cyclic peptides that target to IgE: FcεR1 interaction (Smith et al., Future Med Chem, 2013, 5, 1423-1435; the content of which is incorporated by reference in its entirety,) and anti-IgE receptor antibodies and derivatives.

For example, the polynucleotides of the present invention may be used for inhibiting histamine release as described in U.S. Pat. No. 6,290,957), and reducing circulating IgE as described U.S. Pat. No. 5,543,144; the contents of each of which are herein incorporated by reference in their entirety.

According to the present invention, the omalizumab polynucleotides of the present invention are engineered such that they may comprise one or more coding regions.

The coding regions of the omalizumab polynucleotides may encode any of the regions or portions of the omalizumab antibody. They may also further comprise coding regions not found in the original or parent omalizumab antibody.

The omalizumab polynucleotides of the present invention may be engineered to produce any of the 5 standard classes of immunoglobulins as shown in FIG. 1 using the omalizumab antibody or any of its component parts as a starting molecule.

The omalizumab polynucleotides may also be engineered according to the present invention to produce a variant omalizumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Panitumumab Parent Molecule or Antibody

According to the present invention, panitumumab polynucleotides or constructs and their associated panitumumab compositions are designed to produce the panitumumab antibody, a variant or a portion thereof in vivo.

Panitumumab (synonyms: ABX-EGF; rHuMAb-EGFR, Vectibix) is a fully human monoclonal antibody that binds with high affinity to epidermal growth factor receptor (EGFR) and interferes with signals that might otherwise stimulate growth and survival of the cancer cells. EGFR is a protein that plays an important role in cancer cell signaling and is over-expressed in many human cancers. The inhibition of EGFR mediated growth signal by antibody binding could inhibit the abnormal growth of EGFR expression tumor cells. Panitumumab received approval from the Food and Drug Administration in 2006 as a single agent for the treatment of metastatic colorectal carcinoma with disease progression on or following fluoropyrimidine, oxaliplatin, and irinotecan chemotherapy regimens. Approval is based on progression-free survival in clinical studies.

However, panitumumab treatment may cause severe infusion reactions and dermatological toxicities in patients. As with other antibody based protein drugs, panitumumab treatment may raise the immunogenicity in patients.

Panitumumab is a recombinant, human IgG2 kappa monoclonal antibody that binds specifically to the human epidermal growth factor receptor (EGFR). Panitumumab has an approximate molecular weight of 147 kDa. Panitumumab is produced in genetically engineered mammalian (Chinese Hamster Ovary) cells.

U.S. Pat. No. 6,235,883 disclosed this fully human anti-EGFR antibody. It contains an amino acid sequence of the heavy chain variable region in which a portion of the sequence is encoded by a human V_{H} 4-61 gene, disulfide with an amino acid sequence of the light chain variable region in which a portion of the sequence is encoded by a human Vκ I family gene. The antibody is selected from the hybridoma E7.6.3 (see, also e.g. PCT patent publication NO WO1998050433; Yang et al., Rev Oncol Hematol 2001; 38:17-23; Yang et al., Cancer Res 1999; 59:1236-1243; the contents of each of which are herein incorporated by reference in their entirety).

Certain sequences encoding panitumumab fragments, domains or heavy or light chains are given in Table 23. The table is not an exhaustive list and any fragment or portion of the sequence may be encoded in the panitumumab polynucleotides of the invention.

TABLE 23
Table of Panitumumab Sequences

| | | | SEQ ID |
|---|---|---|---|
| Description | Sequence | Source | NO. |
| Heavy chain | VSGGSVSSGDYYWTWIRQSPGKGL | From | 112 |
| variable | EWIGHIYYSGNTNYNPSLKSRLTISI | U.S. Pat. No. 6,235,883, | |
| region | DTSKTQFSLKLSSVTAADTAIYYCV | SEQ ID NO 37 | |
| | RDRVTGAFDIWGQGTMVTSS | | |
| Light chain | TITCQASQDISNYLNWYQQKPGKAP | From | 113 |
| variable | KLLIYDASNLETGVPSRFSGSGSGTD | U.S. Pat. No. 6,235,883, | |
| region | FTFTISSLQPEDIATYFCQHFDHLPL | SEQ ID NO 38 | |
| | AFGGGTKVEIKRTVAAPSVFIFPPSD | | |
| | EQ | | |
| the amino | VVSGGSVSSGSYYWSWIRQPPGKG | From | 114 |
| acid | LEWIGYIYYSGSTNYNPSLKSRVTIS | U.S. Pat. No. 6,235,883, | |
| sequence | VDTSKNQFSLRLSSVTAADTAVYY | SEQ ID NO 22 | |
| encoded by | CAR | | |
| the V_{H}4-61 | | | |
| gene | | | |
| the amino | TITCQASQDISNYLNWYQQKPGKAP | From | 115 |
| acid | KLLIYDASNLETGVPSRFSGSGSGTD | U.S. Pat. No. 6,235,883, | |
| sequence | FTFTISSLQPEDIATYYCQQYDNLP | SEQ ID NO 20 | |
| encoded by | | | |
| the V_{K}gene | | | |

The EGFR is a transmembrane glycoprotein that is a member of the HER subfamily of type I receptor tyrosine kinases, including EGFR (ErbB1/HER1), HER2 (ErbB2), HER3 (ErbB3), and HER4 (ErbB4). EGFR is a classic membrane-bound tyrosine kinase receptors, whose activation is typically ligand dependent, with the principal ligands being EGF and TGF-α. EGFR is constitutively expressed in normal epithelial tissues, including the skin and hair follicle. EGFR is over-expressed in certain human cancers, including colon and rectum cancers. Interaction of EGFR with its normal ligands (eg, EGF, transforming growth factor-alpha) leads to homo- or heterodimerization and auto-phosphorylation and activation of a series of intracellular pathways. These pathways include the Ras-Raf-MAPK pathway, the PI3K-AKT pathway, the protein kinase C pathway, the STAT pathway, and the src kinase pathway, all of which play important roles in tumor cell proliferation, invasion, migration, and inhibition of apoptosis. EGFR activation does not initiate linear downstream pathway signaling, but rather can activate multiple pathways that cross-connect intracellularly.

Anti-EGFR therapies include monoclonal antibodies (e.g. panitumumab) that recognize the EGFR and small molecule inhibitors of EGFR tyrosine kinase activity (TKIs). Panitumumab binds specifically to EGFR on both normal and tumor cells, and competitively inhibits the binding of ligands for EGFR, therefore prevents it from sending growth signals. Nonclinical studies show that binding of panitumumab to the EGFR prevents ligand-induced receptor autophosphorylation and activation of receptor-associated kinases, resulting in inhibition of cell growth, induction of apoptosis, decreased pro-inflammatory cytokine and vascular growth factor production, and internalization of the EGFR. In vitro assays and in vivo animal studies demonstrate that panitumumab inhibits the growth and survival of selected human tumor cell lines expressing EGFR.

Panitumumab has been used in clinics to treat patients with EGFR-expressing, metastatic carcinoma of the colon or rectum (mCRC), who have progressed on or following treatment with a regimen(s) containing a fluoropyrimidine, oxaliplatin, and irinotecan. Among the patients treated with panitumumab, the membrane staining intensity of EGFR in tumor cells and the percentage of tumor cells with EGFR membrane staining are decreased (see, National Cancer Institute, clinical trials, www.cancer.gov/clinicaltrials/search/results?protocolsearchid=6289704.).

In clinical studies, panitumumab has demonstrated anti-tumor activity in advanced, refractory colorectal cancer (“CRC”). Patients who received panitumumab every two weeks showed a 46 percent decrease in tumor progression rate versus those who received best supportive care alone.

It is shown that EGFR expression levels show no correlation with response to therapy. K-RAS, a small serine-threonine kinase that is farnesylated and inserted into the cell membrane, is an important predictor of response to anti-EGFR mAb therapy. Lievre et al (J Clin Oncol. 2008, 26, 374-379; the content of which is herein incorporated by reference in its entirety) identified a k-ras mutation in 27% of patients, with a response rate of 0% in tumors with mutated k-ras vs. 40% in tumors with wild type k-ras and a increased median overall survival. K-ras mutation testing may be used to guide anti-EGFR based therapeutic decisions.

Panitumumab has also been investigated in clinical studies for treating various solid cancers, such as head and nech cancer (HNC), non-small cell lung cancer (NSCLC), metastatic gallbladder carcinoma (e.g. Markovic and Chung et al., Expert Rev Anticancer ther, 2012, 12, 1149-1159; Riley and Carloss, Oncologis, 2011, 16, 1-2; Grunwald and Hodalgo, J Natl Can Instit., 2003, 95, 851-867; the contents of each of which are herein incorporated by reference in their entirety).

The major concerns of using panitumumab treatment include infusion reactions and dermatological toxicities. Severe infusion reactions occurred in approximately 1% of patients and dermatologic toxicities were reported in 89% of patients and were severe in 12% of patients receiving monotherapy. Most common toxicities (≧20%) are skin toxicities (ie, erythema, dermatitis acneiform, pruritus, exfoliation, rash, and fissures). Furthermore, panitumumab causes increased toxicities with combination chemotherapy, so it is not indicated for use in combination with chemotherapy. Severe infusion reactions included anaphylactic reactions, bronchospasm, and hypotension. Other adverse reactions may include pulmonary fibrosis, photosensitivity, paronychia, hypomagnesemia, fatigue, abdominal pain, nausea, diarrhea, and constipation. As with other antibody based therapeutic proteins, there is potential for immunogenicity for panitumumab treatment. The polynucleotides of the present invention are expected to overcome some if not all of the side effects associated with the commercial antibody.

In one embodiment, the polynucleotides encoding panitumumab may further include one or more modular recognition domain(s) (MRD), generating a targeting antibody-MRD fusion molecule, as described in U.S. Pat. No. 8,557,243, the content of which is herein incorporated by reference in its entirety. The nucleic acids encoding the MRD domain may be linked to the N-terminal of either heavy chain or light chain of the polynucleotide encoding panitumumab. In another aspect, the nucleic acids encoding the MRD domain may be linked to the C-terminal of either heavy chain or light chain of the polynucleotide encoding panitumumab. The MRD may contain in general a peptide sequence that binds to target sites of interests, for example Ang2-binding MRD and IGF-1R-binding MRD as described in U.S. Pat. Nos. 8,557,243 and 8,454,960; the contents of each of which are herein incorporated by reference in their entirety.

Panitumumab, as anti-EGFR therapeutic antibody, has been disclosed for the use of treating metastatic colorectal cancer (e, g. U.S. Pat. Nos. 8,535,670; 7,858,390; US patent publication NOs: US20060216288; and US 20100074909; Yang et al., Crit. Rev. Onco Hematol, 2001, 38, 17-32); non-small cell lung cancer (see e.g. U.S. Pat. Nos. 8,575,191 and 7,858,389); neurological disorder (e.g. U.S. Pat. No. 8,142,782; PCT patent publication NO: WO2013005108); renal carcinoma (e.g. US patent publication NO: 20040033543; PCT patent publication NO: WO2003099205); head and neck carcinoma; heart disease (e.g. US patent publication NO: 20130230581, the contents of each of references are herein incorporated by reference in their entirety).

In some embodiments, the polynucleotides encoding panitumumab may be used as a targeted cancer therapy agent for treating EGFR expressing cancers, such as colorectal cancer, non-small cell lung cancer, head and neck cancer and renal carcinoma. In one aspect, the colorectal cancer may be a KRAS wild-type, metastatic colorectal cancer.

In some embodiments, the polynucleotides of the present invention may be co-administered to a cancer patient with other anti-cancer antibodies, including, but not limited to, trastuzumab, bevacizumab, rituximab, pertuzumab, cetuximab, IMC-1C11, tositumomab, ibirtumomab tiuxetan, EMD 7200, SGN-30, SGN-15, SGN-40, SGN-35, and SGN-17/19.

In one aspect, the polynucleotides of the present invention may be used in combination with other standard chemotherapeutic agents for treating tumors. Said chemotherapeutic agents may include folinic acid (leucovorin)-5FU-oxaliplatin (FOLFOX)4; FOLFIRI; CNU, lomustine, CCNU, cytosine arabinoside, cyclophosphamide, estramustine, hydroxyurea, procarbazine, mitomycin, busulfan, medroxyprogesterone, estramustine phosphate sodium, ethinyl estradiol, estradiol, megestrol acetate, methyltestosterone, diethylstilbestrol diphosphate, chlorotrianisene, testolactone, mephalen, mechlorethamine, chlorambucil, chlormethine, ifosfamide, bethamethasone sodium phosphate, dicarbazine, asparaginase, mitotane, vincristine, vinblastine, etoposide, teniposide, Topotecan, IFN-gamma, irinotecan, campto, irinotecan analogs, carmustine, fotemustine, lomustine, streptozocin, carboplatin, oxaliplatin, BBR3464, busulfan, dacarbazine, mechlorethamine, procarbazine, thioTEPA, uramustine, vindesine, vinorelbine, alemtuzumab, tositumomab, methyl aminolevulinate, porfimer, verteporfin, lapatinib, nilotinib, vandetanib, ZD6474, alitretinoin, altretamine, amsacrine, anagrelide, denileukin diftitox, estramustine and hydroxycarbamide.

In some embodiments, the polynucleotides encoding panitumumab may be used to inhibit EFGR mediating signals in a subject, alone or in combination with other an epidermal growth factor tyrosine kinase inhibitors, such as cetuximab, TheraCIM, EMD 72000, MDX447, gefitinib, lapatinib, erlotinib, PKI-166, canertinib, matuzumab, GW572016, CL-1033, EKB-569, GW2016, EKB-569 (See, e.g. U.S. Pat. No. 8,575,191); kinase inhibitors as described in U.S. Pat. No. 8,557,857; anti-EGFR 565 as described in U.S. Pat. Nos. 7,939,072; 7,887,805; 6,699,473; the contents of each of which are incorporated herein by reference in their entirety).

According to the present invention, the panitumumab polynucleotides of the present invention are engineered such that they may comprise one or more coding regions.

The coding regions of the panitumumab polynucleotides may encode any of the regions or portions of the panitumumab antibody. They may also further comprise coding regions not found in the original or parent panitumumab antibody.

The panitumumab polynucleotides of the present invention may be engineered to produce any of the 5 standard classes of immunoglobulins as shown in FIG. 1 using the panitumumab antibody or any of its component parts as a starting molecule.

The panitumumab polynucleotides may also be engineered according to the present invention to produce a variant panitumumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Ranibizumab Parent Molecule or Antibody

According to the present invention, ranibizumab polynucleotides or constructs and their associated ranibizumab compositions are designed to produce the ranibizumab antibody, a variant or a portion thereof in vivo.

Ranibizumab (synonyms: rhuFab V2) is marketed under the trade name Lucentis.

Ranibizumab is a recombinant humanized IgG1 kappa monoclonal antibody fragment designed for intraocular use. Ranibizumab binds to human vascular endothelial growth factor A (VEGF-A) and inhibits the biologic activity of active forms of human VEGF-A, including the cleaved form (VEGF₁₁₀).

Ranibizumab has been approved by the Food and Drug Administration in 2006 for treating the “wet” (i.e. neovascular) type of age related macular degeneration (AMD or ARMD), a common form of age related vision loss. It is also indicated for the treatment of patients with Macular Edema Following Retinal Vein Occlusion (RVO) and Diabetic Macular Edema (DME).

The most common toxic effects of ranibizumab treatment to the eye are eye pain, vitreous floaters, increased intraocular pressure, conjunctival hemorrhage. Also arterial thromboembolic events have occurred in patients. Consequently, there is a need for improved ranibizumab molecules.

Ranibizumab is a recombinant humanized IgG1 kappa monoclonal antibody fragment (i.e. Fab fragment) derived from the same parent mouse antibody as bevacizumab (Avastin), much smaller than the parent full antibody but having a higher binding affinity to VEGF-A. Ranibizumab is specifically designed for intraocular use. The small size allows it to better penetrate the retina, and thus treat the ocular neovascularization associated with AMD (Lien and Lowman, In: Chemajovsky, 200 Therapeutic Antibodies. Handbook of Experimental Pharmacology 181, Springer-Ver 0 Berlin Heidelberg 131-150; the content of which is incorporated by reference in its entirety.)

Certain sequences encoding ranibizumab fragments, domains or heavy or light chains are given in Table 24. The table is not an exhaustive list and any fragment or portion of the sequence may be encoded in the ranibizumab polynucleotides of the invention.

TABLE 24
Table of Ranibizumab Sequences

| | | SEQ ID |
|---|---|---|
| Description | | NO. |
| Fab-12 variant Y0317/L- | DIQLTQSPSSLSASVGDRVTITCSASQDISN | 116 |
| KAPPA (V-KAPPA(1-107) + | YLNWYQQKPGKAPKVLIYFTSSLHSGVPS | |
| C-KAPPA(108-213 | RFSGSGSGTDFTLTISSLQPEDFATYYCQQ | |
| | YSTVPWTFGQGTKVEIKRTVAAPSVFIFPP | |
| | SDEQLKSGTASVVCLLNNFYPREAKVQW | |
| | KVDNALQSGNSQESVTEQDSKDSTYSLSS | |
| | TLTLSKADYEKHKVYACEVTHQGLSSPVT | |
| | KSFNRGE | |
| Fab-12 variant Y031/Fab- | EVQLVESGGGLVQPGGSLRLSCAASGYDF | 117 |
| 12 variant Y0317 and VH- | THYGMNWVRQAPGKGLEWVGWINTYTG | |
| CH1 (VH(1-123) + | EPTYAADFKRRFTFSLDTSKSTAYLQMNS | |
| CH1(124-215) | LRAEDTAVYYCAKYPYYYGTSHWYFDV | |
| | WGQGTLVTVSSASTKGPSVFPLAPSGTAA | |
| | LGCLVKDYFPEPVTVSWNSGALTSGVHTF | |
| | PAVLQSSGLYSLSSVVTVPSSSLGTQTYIC | |
| | NVNHKPSNTKVDKKVEPK | |
| Fab-12 variant | EVQLVESGGGLVQPGGSLRLSCAASGYDF | 118 |
| Y0317/VH-CH1 (VH(1-123) + | THYGMNWVRQAPGKGLEWVGWINTYTG | |
| CH1(124-215) | EPTYAADFKRRFTFSLDTSKSTAYLQMNS | |
| | LRAEDTAVYYCAKYPYYYGTSHWYFDV | |
| | WGQGTLVTVSSASTKGPSVFPLAPSGTAA | |
| | LGCLVKDYFPEPVTVSWNSGALTSGVHTF | |
| | PAVLQSSGLYSLSSVVTVPSSSLGTQTYIC | |
| | NVNHKPSNTKVDKKVEPK | |
| Fab-12 lcz8_L\|Fab-12 | DIQLTQSPSSLSASVGDRVTITCSASQDISN | 119 |
| variant Y0317/L-KAPPA | YLNWYQQKPGKAPKVLIYFTSSLHSGVPS | |
| (V-KAPPA(1-107) + C- | RFSGSGSGTDFTLTISSLQPEDFATYYCQQ | |
| KAPPA(108-213) | YSTVPWTFGQGTKVEIKRTVAAPSVFIFPP | |
| | SDEQLKSGTASVVCLLNNFYPREAKVQW | |
| | KVDNALQSGNSQESVTEQDSKDSTYSLSS | |
| | TLTLSKADYEKHKVYACEVTHQGLSSPVT | |
| | KSFNRGE | |

VEGF-A has been shown to be involved in angiogenesis and increasing vascular permeability, which is believed to contribute the progression of the neovascular form of AMD.

Ranibizumab binds to and inhibits the biologic activity of active forms of human VEGF-A, including the biologically active, cleaved form (VEGF₁₁₀). Ranibizumab binds to the receptor-binding site of active forms of VEGF-A, including VEGF₁₁₀, preventing the interaction of VEGF-A with its receptors (VEGFR1 and VEGFR2) on the surface of endothelial cells, therefore inhibiting cell proliferation, vascular leakage and new blood vessel formation.

Ranibizumab can be used for the treatment of patients with macular edema after retinal vein occlusion, age-related macular degeneration (wet, neovascular), and diabetic macular edema (DME).

AMD is a medical condition that usually affects older adults and results in a loss of vision in the center of the visual field (the macula) because of damage to the retina. It occurs in “dry” and “wet” forms. It is a major cause of blindness and visual impairment in older adults (>50 years).

The proliferation of abnormal blood vessels in the retina is stimulated by VEGF. Anti-angiogenics or anti-VEGF agents can cause regression of the abnormal blood vessels and improve vision when injected directly into the vitreous humor of the eye. Ranibizumab in clinical studies have been proved to be effective in treating AMD, particularly for wet AMID.

Macular Edema occurs when fluid and protein deposits collect on or under the macula of the eye (a yellow central area of the retina) and causes it to thicken and swell (edema). The swelling may distort a person's central vision, as the macula is near the center of the retina at the back of the eyeball. Macular edema may be caused by many conditions such as Retinal vein occlusion (RVO), which is a common vascular disorder of the retina, and diabetes. Ranibizumab in clinical trials have been proved to be effective in treating macular edema (Garnock et al, drugs, 2011, 71, 455-463; the content of which is incorporated herein by reference in its entirety.)

Anti-VEGF antibodies have been disclosed to treat the diseases associated with the activity and/or overproduction of VEGF, such as in US patent publication NOs: 20100322931; and PCT patent publication NO: WO2010148223; the contents of each of which are incorporated herein be reference in their entirety.

In some embodiments, the polynucleotides encoding ranibizumab may be used as a vascular endothelial growth factor inhibitor, to neutralize VEGF activity in disease condition. In some aspect, the polynucleotides encoding ranibizumab neutralize \′EGF by at least 30%, by at least 40%, by at least 50%, by at least 60%, by at least 70%, by at least 80%, by at least 90?/k, or by a percentage ranging between any of the values.

In certain embodiments, the polynucleotides encoding ranibizumab are useful in the treatment of tumors in which angiogenesis plays an important role in tumor growth, including cancers and benign tumors. Examples of cancer to be treated herein include, but are not limited to, carcinoma, lymphoma, blastoma, sarcoma, and leukemia. See, e.g. US Patent publication US20100221247; US20070134244; US20120130144; and PCT patent publication NO: WO2011133668; the contents of each of which are incorporated herein be reference in their entirety.

The present disclosure encompasses anti-angiogenic therapy, a cancer treatment strategy aimed at inhibiting the development of tumor blood vessels required for providing nutrients to support tumor growth. Because angiogenesis is involved in both primary tumor growth and metastasis, the antiangiogenic treatment provided by the disclosure is capable of inhibiting the neoplastic growth of tumor at the primary site as well as preventing metastasis of tumors at the secondary sites.

Anti-VEGF antibodies are useful in treating eye diseases in which VEGF signal make a significant contribution to the pathogenesis, such as age related macular degeneration as described US20070134244; vascularized retinal pigment epithelial detachment as described in US20130004486; also in US20130295094; the contents of each of which are herein incorporated by reference in their entirety.

In some embodiments, the polynucleotides encoding ranibizumab may be used to treat an eye disease, in particular an angiogenic eye disorder. Examples of eye diseases may include, but are not limited to, dry and wet age related macular degeneration, macular edema following retinal vein occlusion (RVO); diabetic macular edema; vascularized retinal pigment epithelial detachment (vPED); diabetic retinopathy; central retinal vein occlusion and corneal neovascularization.

In certain embodiments, the polynucleotides encoding ranibizumab may be used with one or more other VEGF antagonists. VEGF antagonists include molecules which interfere with the interaction between VEGF and a natural VEGF receptor, e.g. molecules which bind to VEGF or a VEGF receptor and prevent or otherwise hinder the interaction between VEGF and a VEGF receptor. Specific exemplary VEGF antagonists include anti-VEGF antibodies, anti-VEGF receptor antibodies, and VEGF receptor-based chimeric molecules (also referred to herein as “VEGF-Traps”).

According to the present invention, the ranibizumab polynucleotides of the present invention are engineered such that they may comprise one or more coding regions.

The coding regions of the ranibizumab polynucleotides may encode any of the regions or portions of the ranibizumab antibody. They may also further comprise coding regions not found in the original or parent ranibizumab antibody.

The ranibizumab polynucleotides of the present invention may be engineered to produce any of the 5 standard classes of immunoglobulins as shown in FIG. 1 using the ranibizumab antibody or any of its component parts as a starting molecule.

The ranibizumab polynucleotides may also be engineered according to the present invention to produce a variant ranibizumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Rituximab Parent Molecule or Antibody

According to the present invention, rituximab polynucleotides or constructs and their associated rituximab compositions are designed to produce the rituximab antibody, a variant or a portion thereof in vivo.

Rituximab (synonym: anti-CD20) is a chimeric human-mouse monoclonal antibody recognizing the CD 20 antigen expressed on B-lymphocytes. Rituximab is a type I anti-CD20 antibody, which is potent in inducing complement mediated cytotoxicity (CDC) and antigen dependent cell-mediated cytotoxicity (ADCC). Rituximab is approved by the Food and Drug Administration (FDA) for treating CD-20 positive non-hodgkin's lymphoma (NHL), chronic lymphocytic leukemia (CLL), rheumatoid arthritis (RA) and other diseases.

Rituximab may cause serious and sometimes life-threatening side effects in patients, such as cardiac arrhythmias. The intravenous infusion reactions during and/or after the treatment are the most common adverse reactions. Extremely high level of immunotherapeutic antibody is required to deplete circulating tumor cells, which is beyond the tolerable toxicity caused by administrating antibodies into a patient. Thus, it is necessary to develop a novel method to produce this therapeutic antibody or variants thereof in vivo in patients to increase the treatment efficacy and reduce its toxicity.

Rituximab is a genetically engineered chimeric human-murine monoclonal antibody directly against the CD20 antigen found on the surface of normal and malignant B-lymphocytes. This antibody is an IgG1 kappa immunoglobulin containing murine light- and heavy chain variable region sequences and human gamma I heavy-chain and kappa light-chain constant region sequences. Rituximab is a dimer, composed of two heavy chains of 451 amino acids and two light chains of 213 amino acids and has an approximate molecular weight of 145 kD. The sequences of both heavy and light chain are listed in Table 25.

Rituximab was genetically engineered using the murine 2B8 antibody, as disclosed in U.S. Pat. No. 5,736,137 (Andersen, et. al.) issued on Apr. 17, 1998, assigned to IDEC Pharmaceuticals Corporation, and U.S. Pat. No. 7,422,739, assigned to Biogen Idec, the contents of each of which are herein incorporated by reference in their entirety.

This antibody may be further modified for increased binding affinity and effector function, e.g. as described in US patent publication NOs: US20050123546, US20070071745; PCT publication NOs: WO 2005/044859 and WO2007/031875, the contents of each of which are herein incorporated by reference in their entirety.

Certain sequences of modified polynucleotides encoding rituximab or variants are disclosed in copending US Publication 20120237975, the contents of which are incorporated herein by reference in their entirety.

Certain sequences encoding rituximab fragments, domains or heavy or light chains are given in Table 25. The table is not an exhaustive list and any fragment or portion of the sequence may be encoded in the rituximab polynucleotides of the invention.

TABLE 25
Table of Rituximab Sequences

| | | SEQ |
|---|---|---|
| | | ID |
| Description | Sequence | NO. |
| Rituxmab | QVQLQQPGAELVKPGASVKMSCKASGYTFTSYN | 120 |
| heavy chain | MHWVKQTPGRGLEWIGAIYPGNGDTSYNQKFK | |
| chimeric | GKATLTADKSSSTAYMQLSSLTSEDSAVYYCAR | |
| | STYYGGDWYFNVWGAGTTVTVSAASTKGPSVF | |
| | PLAPSSKSTSGGTAALGCLVKDYFPEPVTVSWNS | |
| | GALTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTCVV | |
| | VDVSHEDPEVKFNWYVDGVEVHNAKTKPREEQ | |
| | YNSTYRVVSVLTVLHQDWLNGKEYKCKVSNKA | |
| | LPAPIEKTISKAKGQPREPQVYTLPPSRDELTKNQ | |
| | VSLTCLVKGFYPSDIAVEWESNGQPENNYKTTPP | |
| | VLDSDGSFFLYSKLTVDKSRWQQGNVFSCSVMH | |
| | EALHNHYTQKSLSLSPGK | |
| Rituximab | QIVLSQSPAILSASPGEKVTMTCRASSSVSYIHWF | 121 |
| light chain | QQKPGSSPKPWIYATSNLASGVPVRFSGSGSGTS | |
| chimeric | YSLTISRVEAEDAATYYCQQWTSNPPTFGGGTKL | |
| | EIKRTVAAPSVFIFPPSDEQLKSGTASVVCLLNNF | |
| | YPREAKVQWKVDNALQSGNSQESVTEQDSKDST | |
| | YSLSSTLTLSKADYEKHKVYACEVTHQGLSSPVT | |
| | KSFNRGEC | |

Rituximab binds to the CD20 antigen, which is predominantly expressed on pre-B and mature B cells, and on >90% of B-cell non-Hodgkin's lympohomas (NHL), but not found on hematopoietic stem cells, pro-B cells, normal plasma cells or other normal tissues. CD20 regulates an early step(s) in the activation process for cell cycle initiation and differentiation, and possibly functions as a calcium ion channel. It is also known that CD20 is not shed from the cell surface and does not internalize upon antibody binding. The antibody Rituximab tends to stick to one side of cancerous B cells, forming a cap and drawing proteins over to that side, leading to selective killing of B-cells by natural killer cells (NK). In particular, the Fab regions of rituximab bind to the CD20 antigen on B lymphocytes, while the Fc domain recruits antibodies and complements to mediate antibody dependent cellular cytotoxicity (ADCC) and complement dependent cytotoxicity (CDC), inducing cell lysis (Reff, M E, et. al, Blood, 1994, 83, 435-445, the content of which is herein incorporated by reference in its entirety). Rituximab binds to amino acid residues 170-173 and 182-185 on CD20, which are physically close to each other as a result of a disulfide bond between residues 167 and 183 (e.g. Binder et al., Blood, 2006, 108, 1975-1978; the content of which is herein incorporated by reference in its entirety).

In addition, Rituximab affects the interferon (IFN) I response genes as described by Verweij C L et al., Discov Med, 2011, 12, 229-236; the content of which is herein incorporated by reference in its entirety.

Rituximab is approved for the treatment of patients with B cell lymphomas, rheumatoid arthritis and other diseases.

Non-Hodgkin's lymphoma (NHL) refers to any of a large group of cancers of lymphocytes (white blood cells). Non-Hodgkin lymphomas can occur at any age and are often marked by lymph nodes that are larger than normal, fever, and weight loss. Non-Hodgkin's lymphoma (NHL) may include mantle cell lymphoma, diffuse large B-cell lymphoma (DLBCL), chronic lymphocytic leukemia (CLL)/small lymphocytic lymphoma (SLL), or follicular lymphoma (FL).

Rituximab is used to treat relapsed or refractory low-grade or follicular, CD20-positive, B-cell NHL as a single agent; or to treat previously untreated follicular, CD20-positive, B-cell NHL in combination with first line chemotherapy, or to treat previously untreated diffuse large B-cell, CD20-positive NHL in combination with CHOP or other anthracycline-based chemotherapy regimens. Rituximab may also be used for patients achieving a complete or partial response to rituximab in combination with chemotherapy, as single-agent maintenance therapy; or for patients with nonprogressing (including stable disease), low-grad, CD20-positive, B-cell NHL as a single agent after first-line CVP chemotherapy.

Chronic lymphocytic leukemia/small lymphocytic lymphoma (CLL/SLL) represents the most prevalent adult leukemia, with an incidence rate of 2 to 6 cases per 100 000 people per year. The median survival is highly variable with some patients exhibiting an indolent natural history, whereas others develop aggressive disease with a survival of less than 2 to 3 years. Therapy for CLL has evolved significantly from 1970 when alkylator-based therapy such as chlorambucil or cyclophosphamide was used. Treatment intensification in CLL from alkylator to fludarabine and cyclophosphamide combinations resulted in increased cellular immune suppression and myelosuppression.

In addition, chemotherapy intensification did not greatly improve treatment outcomes in patients with high-risk genomic features. Attempts to intensify chemotherapy beyond fludarabine/alkylator-based combinations have been pursued with enhanced toxicity but little evidence of clinical benefit. The clinical application of therapeutic monoclonal antibodies more than the past decade has impacted the therapeutic approach to CLL and point to potential opportunities in the future with other targeted therapies currently being explored (Jaglowski et al., Blood, 2010, 116, 3705-3714; the content of which is herein incorporated by reference in its entirety).

Rituximab is indicated for untreated and previously treated CD20-positive CLL; combined therapy with fludarabine and cyclophosphamide (FC).

Rheumatoid arthritis is a chronic autoimmune disease that causes pain, stiffness, swelling and limited motion and function of many joints. While RA can affect any joint, the small joints in the hands and feet tend to be involved most often. Inflammation sometimes can affect organs as well, for instance, the eyes or lungs. One possible mechanism of RA is that the immune system of patients is abnormal and attacks the body and creates inflammation. There is no cure for RA. Current treatments can lessen the symptoms and slow the dysfunction of the joints.

Rituximab is approved by the Food and Drug Administration to treat active rheumatoid arthritis unresponsive to DMARDs (Disease-Modifying Antirheumatic Drugs) and anti-TNF-alpha agents. Rituximab is used to treat rheumatoid arthritis in combination with methotrexate (MTX) (e.g. Mease P J et al., J Rheomatol., 2010, 37, 917-927; also reviewed by Rosman et al., BMC Medcine, 2013, 11, 88; the contents of each of which are herein incorporated by reference in their entirety).

Rituximab is beneficial for other off-label indications in patients with other autoimmune diseases (such as systemic lupus erythematosus (SLE)), Castleman's disease, Granulomatosis with polyangiitis (GP) (previously wegener Granulomatosis), and Microscopic Polyangiitis. Rituximab may also be used to treat Immune Thrombocytopenic Purpura (ITP), refractory autoimmune hemolytic anemia, Castleman's disease, thrombocytopenia, arthritis-related SLE, systemic sclerosis (SyS) and corticosteroid refractory pemphigus vulgaris (e.g. Cianchini G et al., J Am Acad Dermatol, 2012, 67, 617-622; Palau et al., Int J Gen Med, 2010, 3, 305-311; Wallace D J, BMC Medcine, 2010, 37, 558-567; also reviewed by Rosman et al., BMC Medcine, 2013, 11, 88; the contents of each of which are herein incorporated by reference in their entirety).

The polynucleotides of the present invention may be used to treat any of the disorders or diseases taught herein.

Rituximab may be used to treat B-cell lymphomas, including relapsed indolent lymphoma (McLaughlin P et al., J Clin Oncol. 1998, 16, 2825-33, the content of which is incorporated herein by reference in its entirety), non-hodgkin's lymphoma and chronic lymphocytic leukemia (see, e.g. U.S. Pat. Nos. 5,776,456; 5,843,430; 6,846,476; 6,682,734; 6,455,043; 6,399061; 7,381,560; 7,682,612; 7,744,877; 8,206,711; US patent publication NO: US20060029543; the contents of each of which are incorporated herein by reference in their entirety), alone or in combination with other chemotherapy medicines such as chemotherapy medicines fludarabine and cyclophosphamide. Rituximab may also be used to treat rheumatoid arthritis (Edwards J C et al., N Engl J Med. 2004, 350, 2572-81, the content of which is incorporated herein by reference in its entirety), with methotrexate, to reduce the signs and symptoms of moderate to severe active RA in adults, after treatment with at least one other medicine such as a Tumor Necrosis Factor (TNF) antagonist has been used and did not work well enough; or Granulomatosis with Polyangiitis (GPA) (previously Wegener's Granulomatosis) and Microscopic Polyangiitis (MPA), with glucocorticoids.

In some embodiments, the polynucleotides of the present invention may be used to treat hematological cancer (e.g. non-hodgkin lymphoma (NHL) and chronic lymphocytic leukemia (CLL)), autoimmune disease (e.g. Rheumatoid arthritis); anti-rejection after organ transplant and other B cell related diseases and conditions.

In some embodiments, the polynucleotides of the present invention may be used in combination with other anti-CD20 antibodies to treat CD20 positive cancers and/or other clinical conditions. The anti-CD 20 antibodies include, but are not limited to, ocrelizuman (2H7.v16, PRO-70769, R-1594), DXL 625, ocaratuzumab (AME-133), TRU-015, ofatumumab (2F2), tositumomab, 11B8 (as disclosed in WO 2004035607), AT80 (as disclosed in WO2009030368), humanized B-Lyl antibody (a chimeric humanized antibody as disclosed in WO2005044859), obinutuzumab (GA101), HI47 Ig G3 (ECACC, hybridoma), 2C6 IgG1 (as disclosed in WO2004056312), 2F2 IgG1 (HuMax-CD20) (as disclosed in WO2004035607 and WO2005103081), 2H7 (as disclosed in WO2004056312), Veltuzumab, anti-CD20 antibodies as described in U.S. Pat. Nos. 8,465,741; 8,097,713; 8,057,793; 7,879,984; and US patent publication NOs: US20130195846; US20090136516; US20090130089; the contents of each of which are herein incorporated by reference in their entirety. The combination of anti-CD20 antibodies comprising the polynucleotides of the present invention may also be used to treat Other disease such as inflammatory bowel disease (IBD), sjogren's syndrome, polychondritis or mononeuritis multiplex (e.g. US patent publication NOs: US20060233797; US20060062787; US20060002930; the contents of each of which are incorporated by reference in their entirety).

In one aspect, such anti-CD20 antibodies may be encode by the polynucleotides and co-administrated with the polynucleotides encoding rituximab. The nuclei acid sequence may be modified and codon optimized using the known nucleic acid sequences, for example those described in U.S. Pat. No. 8,097,713, the content of which is incorporated herein by reference in its entirety.

In some embodiments, the polynucleotides encoding Rituximab may be used, in combination with other B-cells depleting antibodies, to deplete B cells for treating a clinical conditions such as B cell malignancies (e.g. chronic lymphocytic leukemia), autoimmune diseases, organ transplantation and serious infections. The B cell depleting antibodies may include, but are not limited to, antibodies against CD19, CD22, CD23, CD27, CD37, CD40, CD53, CD72, CD73, CD74, CDw78, CD79a, CD79b, CD80, CD81, CD82, CD83, CDw84, CD85 and CD86 (e.g. U.S. Pat. Nos. 6,896,885; 7,718,425; US patent publication NOs: US20130309224; 20130295005; US20130266561, US20110305631; US20110300066; US20110002934; and US20080213260; the contents of each of which are incorporated herein by reference in their entirety).

In some embodiments, the polynucleotides encoding Rituximab may be used for treating hematological cancer in combination with other anti-tumor monoclonal antibodies. As non-limiting examples, said anti-tumor monoclonal antibodies may be trastuzumab (anti-Her-2), cetuximab (anti-Her-1), bevacizumab, edrecolomab, panitumumab or alemtuzumab, anti-CD22 antibodies as described in U.S. Pat. No. 7,837,995; anti-CD19 antibodies as described in U.S. Pat. No. 7,837,995; the contents of each of which are incorporated by reference in their entirety. In one aspect, said other monoclonal antibodies may be encoded by the polynucleotides similar to the polynucleotides of the present invention.

In some embodiments, the polynucleotides of the present invention may be used to treat hematological cancers/malignancies, in combination with other cytotoxic, chemotherapeutic and/or anti-cancer agents (e.g. US patent publication NO: US20090209606; the content of which is incorporated by reference in its entirety). As non-limiting examples, the anti-cancer agents may be cytokine IL-15 (e.g. PCT patent publication WO2013076183); CHOP (cyclophosphamide, hydroxydaunorubicin/doxorubicin, vincristine, and prednisone/prednisolone) (U.S. Pat. No. 8,557,244) for aggressive non-hodgkin's lymphoma; protein kinase modulators (U.S. Pat. No. 8,541,461); fludarabine and/or cyclosphosphamide for chronic lymphocytic leukemia (CLL) (U.S. Pat. No. 8,206,711); PI3 Kinase inhibitors (US patent publication 20130064812); a proteasome inhibitor (US20120219549); an BCL-2 active agent (US patent publication 20110287006); a CHK1 inhibitor (U520100226917); an Aurora kinase inhibitor (US20100183601); a BLyS antagonist (US20100143352); Apo2L/TRAIL polypeptide as death receptor ligands (U520090317384); ICE, Mitozantrone, Cytarabine, DVP, ATRA, Idarubicin, hoelzer chemotherapy regime, La La chemotherapy regime, ABVD, CEOP, 2-CdA, FLAG and IDA with or without subsequent G-CSF treatment, VAD, M and P, C-weekly, ABCM, MOPP and DHAP (US patent publication 20120251535); the contents of each of which are incorporated herein by reference in their entirety.

In some embodiments, the polynucleotides of the present invention may be used to treat autoimmune diseases alone or in combination with other therapeutic agents (see, e.g. U.S. Pat. No. 7,074,403; US patent publication NOs: US20090214561; US20060240008; US20060134111; and US20050271658, the contents of each of which are incorporated by reference in their entirety). As non-limiting examples, the polynucleotides encoding Rituximab may be used for treating vasculitis with human glucocorticosteroid (e.g. U.S. Pat. No. 8,545,843); rheumatoid arthritis with anti-IL-6R antibodies or methotrexate (e.g. U.S. Pat. Nos. 8,080,248; and 7,820,161); autoimmune diseases (selected from the group consisting of acute idiopathic thrombocytopenic purpura, chronic idiopathic thrombocytopenic purpura, myasthenia gravis, lupus erythematosus, Sjogren's syndrome and rheumatoid arthritis) with sphingomyelin (e.g. U.S. Pat. No. 7,683,044); autoimmune and immune dysfunction diseases with anti-CD-74 antibodies (e.g. US patent publication 20130295005); multiple sclerosis (e.g. US patent publication NOs: 20130084289; 20120225070; and 20110008336); lupus with hormone steroids (e.g. methylprednisolone, prednisone, mycophenolate mofetil, methotrexate, hydroxychloroquine, chloroquine, quinacrine, azathiprine, or 6-mercaptopurine)(e.g. US20100303810); the contents of each of which are incorporated herein by reference in their entirety.

In some embodiments, the polynucleotides of the present invention may be used to treat infections such as viral infections. As non-limiting examples, Rituximab antibodies generated by the polynucleotides of the present invention may be used to treat viral infections in combination with other B-cell depleting antibodies such as anti-CD10, CD19, CD21, CD22, CD23, CD24, CD37, CD53, CD72, CD73, CD74, CDw75, CDw76, CD77, CDw78, CD79a, CD79b, CD80, CD81, CD82, CD83, CDw84, CD85 and CD86 (see, e.g. U.S. Pat. Nos. 7,718,425; 6,306,393; 6,183,744; US patent publication NOs: 20110008250; US20080233128; the contents of each of which are incorporated herein by reference in their entirety).

In some embodiments, Rituximab antibodies generated by the polynucleotides of the present invention may be used to treat other diseases such as inflammatory bowel disease (IBD) (e.g. US patent publication NO: US20060233797), sjogren's syndrome (e.g. US patent publication NO: US20060062787); and polychondritis or mononeuritis multiplex (e.g. US patent publication NO: US20060002930); the contents of each of which are incorporated herein by reference in their entirety.

In some embodiments, the polynucleotides of the present invention may be used to reduce the risk of relapse of a B-cell-related disease in a patient receiving a bone marrow or peripheral blood stem cell transplant (e.g. US patent publication NO: 20110165159, the content of which is incorporated herein by reference in its entirety).

According to the present invention, the rituximab polynucleotides of the present invention are engineered such that they may comprise one or more coding regions.

The coding regions of the rituximab polynucleotides may encode any of the regions or portions of the rituximab antibody. They may also further comprise coding regions not found in the original or parent rituximab antibody.

The rituximab polynucleotides of the present invention may be engineered to produce any of the 5 standard classes of immunoglobulins as shown in FIG. 1 using the rituximab antibody or any of its component parts as a starting molecule.

The rituximab polynucleotides may also be engineered according to the present invention to produce a variant rituximab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Tocilizumab Parent Molecule or Antibody

According to the present invention, tocilizumab polynucleotides or constructs and their associated tocilizumab compositions are designed to produce the tocilizumab antibody, a variant or a portion thereof in vivo.

Tocilizumab (synonyms: atlizumab) is a recombinant, humanized monoclonal antibody against human interleukine 6 receptor (IL-6R). Tocilizumab is an immunosuppressive drug, mainly for the treatment of rheumatoid arthritis (RA) and systemic juvenile idiopathic arthritis, a severe form of RA in children.

Tocilizumab is a recombinant, humanized, anti-human interleukin 6 (IL-6) receptor monoclonal antibody. The light chain is made up of 214 amino acids. The heavy chain is made up of 448 amino acids. The four polypeptide chains are linked intra- and inter-molecularly by disulfide bonds.

As disclosed in U.S. Pat. No. 7,479,543, this chimeric antibody to human interleukin 6 receptor has light chains each having a human light chain constant Kc and an light chain variable region (V region) of a mouse monoclonal antibody to human IL-6R; and heavy chains (H chains) each having a human H chain constant r γ-1C region, and H chain V region of a mouse monoclonal antibody to human IL-6R (also in U.S. Pat. Nos. 5,795,965; and 5,817,790; the contents of each of which are herein incorporated by reference in their entirety.) The partial amino acid sequences of heavy and light chain are listed in Table 26.

Other variants and/or derivatives from the same parent antibodies are also encompassed in the present invention such as the subtypes of humanized anti-IL-6R antibodies described in U.S. Pat. No. 8,398,980 (also in US20130209456), and mutated tocilizumab with a high affinity as described in U.S. Pat. No. 8,562,991; the contents of each of which are herein incorporated by reference in its entirety.

Certain sequences encoding tocilizumab fragments, domains or heavy or light chains are given in Table 26. The table is not an exhaustive list and any fragment or portion of the sequence may be encoded in the tocilizumab polynucleotides of the invention.

TABLE 26
Table of Tocilizumab Sequences

| | | | SEQ ID |
|---|---|---|---|
| Description | Sequence | Source | NO. |
| Heavy chain | MRVLILLWLFTAFPGILSDVQLQESGPVL | From | 122 |
| varial region | VKPSQSLSLTCTVTGYSITSDHAWSWIRQ | U.S. Pat. No. 7,479,543 | |
| | FPGNKLEWMGYISYSGITTYNPSLKSRISI | SEQ ID NO | |
| | TRDTRDTSNQFFLQLNSVTTGDTSTYYC | 31 | |
| | ARSLARTAMDYWGQGTTSVTVSS | | |
| Light chain | MVSSAQFLGLLLLCFQGTRCDIQMTQTTS | From | 123 |
| varial region | SLSASLGDRVTISCRASQDISSYLNWYQQ | U.S. Pat. No. 7,479,543 | |
| | KPDGTIKLLIYYTSRLHSGVPSRFSGSGTD | SEQ ID NO | |
| | YSLTINNLEQEDIATYFCQQGNTLPYTFG | 29 | |
| | GTKLEIN | | |

Interleukin (IL)-6, a cytokine, plays essential roles not only in the immune response, but also in hematopoiesis and the central nervous system. Deregulated production of IL-6 has been implicated in the pathogenesis of many disorders such as chronic inflammatory autoimmune diseases (e.g. rheumatoid arthritis (RA), systemic onset juvenile idiopathic arthritis (soJIA), Crohn's disease (CD) and systemic lupus erythematosus (SLE)), multiple myeloma and prostate cancer. Furthermore, IL-6 activities can explain many symptoms of these diseases. More importantly, serum levels of IL-6 are correlated with disease activity.

IL-6 signal is mainly mediated by binding the IL-6 receptor. IL-6 binds to either membrane-bound or soluble IL-6R, and this complex in turn binds to the 130 gp signal transducer. This process enhances the inflammatory cascade, inducing angiogenesis and amplifying the activity of adhesion molecules and the activation of osteoclasts.

Tocilizumab binds specifically to soluble as well as membrane-bound IL-6 receptors (sIL-6R and mIL-6R), and has been shown to inhibit IL-6-mediated signaling through these receptors.

Tocilizumab is indicated for the treatment of adult patients with moderately to severely active rheumatoid arthritis (RA) who have had an inadequate response to one or more Disease-Modifying Anti-Rheumatic Drugs (DMARDs). It is also indicated for the treatment of active polyarticular juvenile idiopathic arthritis (PJIA) and active systemic juvenile idiopathic arthritis (SJIA) in patients 2 years of age and older.

In Japan, tocilizumab is also approved for the treatment of castleman's disease, a rare benign B cell tumor.

The most common side effects of tocilizumab include upper respiratory tract infections, nasopharyngitis, headache, hypertension and increased ALT.

In some embodiments, the polynucleotides of the present invention may be used as IL-6 antagonist, inhibiting IL-6 mediated biological activities. In one aspect, it may be used together with other IL-6 antagonist such as Remicade, Zenapx, sirukumab, Elsilimomab, an anti-IL-6 monoclonal antibody.

In some embodiments, the polynucleotides of the present invention may be used to treat an IL-6R associated diseases.

IL-6 associated diseases may include, but are not limited to, acute chronic inflammatory diseases and autoimmune diseases: nephritis, mesangial proliferative nephritis, Crohn's disease, ulcerative colitis, pancreatitis, juvenile idiopathic arthritis or systemic juvenile idiopathic arthritis, vasculitis, Kawasaki disease, rheumatoid arthritis, systemic erythematosus, psoriasis, Sjogren syndrome, adult Still's disease; neoplasmic diseases: multiple myeloma, Castleman's disease, malignant lymphoma, renal cancer; infectious diseases: infection with HIV, infection with EBV; and cachexia: cachexia.

In some embodiments, the polynucleotides of the present invention may be used as monotherapy or in combination with conventional DMARDs in adult patients with moderate to severe rheumatoid arthritis. In some aspects, it may be used to treat juvenile idiopathic arthritis and systemic idiopathic arthritis.

According to the present invention, the tocilizumab polynucleotides of the present invention are engineered such that they may comprise one or more coding regions.

The coding regions of the tocilizumab polynucleotides may encode any of the regions or portions of the tocilizumab antibody. They may also further comprise coding regions not found in the original or parent tocilizumab antibody.

The tocilizumab polynucleotides of the present invention may be engineered to produce any of the 5 standard classes of immunoglobulins as shown in FIG. 1 using the tocilizumab antibody or any of its component parts as a starting molecule.

The tocilizumab polynucleotides may also be engineered according to the present invention to produce a variant tocilizumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Tositumomab Parent Molecule or Antibody

According to the present invention, tositumomab polynucleotides or constructs and their associated tositumomab compositions are designed to produce the tositumomab antibody, a variant or a portion thereof in vivo.

Tositumomab (synonyms: Ig gamma-1 chain C region; anti-B1 antibody) is a cell specific anti-CD20 antibody, mainly used to treat non-Hodgkin's lymphoma and lymphocytic leukemia, in particular, for the treatment of CD20 antigen expressing relapsed or refractory, low grade, follicular, or transformed non-Hodgkin's lymphoma, including patients with rituximab-refractory non-Hodgkin s lymphoma. Tositumomab is usually applied with a sequential infusion followed by iodine tositumomab, which is the same antibody covalently bound to the radionuclide iodine-131. The tositumomab/iodine-131 tositumomab regimen (also called the Bexxar therapeutic regimen), as an antineoplastic radioimmunotherapeutic monoclonal antibody-based regimen, has established the efficacy in treating relapsed or chemotherapy/rituxan refractory follicular lymphoma in patients.

Tositumomab is a human-murine chimeric IgG2a lambda (λ) antibody against human antigen CD20, which is a transmembrane phosphoprotein expressed on pre-B-lymphocytes and mature B lymphocytes. Tositumomab is a dimer, containing 2 heavy chains of 451 residues and 2 mouse monoclonal B1R1gammer2a-chain, disulfide with mouse monoclonal B1R1λ chain with 220 residues.

Tositumomab was humanized from the mouse antibody anti-B1 (obtained from the Hall 299-15 cell line) by Kaminski and Wahl et al (see, e.g. U.S. Pat. Nos. 5,595,721; 5,843,398; 6,015,542; 6,090,365; 6,287,537; 6,565,827; the contents of each which are incorporated herein by reference in their entirety.) This chimeric antibody comprises the B1 antigen-binding domain (CD20 binding) and a human Fc and hinge region. The amino acid sequences of chimeric heavy chain and light chain are listed in 26.

Certain sequences encoding tositumomab fragments, domains or heavy or light chains are given in Table 27. The table is not an exhaustive list and any fragment or portion of the sequence may be encoded in the tositumomab polynucleotides of the invention.

TABLE 27
Table of Tositumomab Sequences

| | | SEQ |
|---|---|---|
| Description | Sequence | ID NO. |
| Human-mouse | QAYLQQSGAELVRPGASVKMSCKASGYTFTSYNMHWV | 124 |
| chimeric anti- | KQTPRQGLEWIGAIYPGNGDTSYNQKFKGKATLTVDKS | |
| CD20 Heavy | SSTAYMQLSSLTSEDSAVYFCARVVYYSNSYWYFDVW | |
| chain 1 | GTGTTVTVSGPSVFPLAPSSKSTSGGTAALGCLVKDYFP | |
| | EPVTVSWNSGALTSGVHTFPAVLQSSGLYSLSSVVTVPS | |
| | SSLGTQTYICNVNHKPSNTKVDKKAEPKSCDKTHTCPPC | |
| | PAPELLGGPSVFLFPPKPKDTLMISRTPEVTCVVVDVSHE | |
| | DPEVKFNWYVDGVEVHNAKTKPREEQYNSTYRVVSVL | |
| | TVLHQDWLNGKEYKCKVSNKALPAPIEKTISKAKGQPR | |
| | EPQVYTLPPSRDELTKNQVSLTCLVKGFYPSDIAVEWES | |
| | NGQPENNYKTTPPVLDSDGSFFLYSKLTVDKSRWQQGN | |
| | VFSCSVMHEALHNHYTQKSLSLSPGK | |
| Human-mouse | QIVLSQSPAILSASPGEKVTMTCRASSSVSYMHWYQQKP | 125 |
| chimeric anti- | GSSPKPWIYAPSNLASGVPARFSGSGSGTSYSLTISRVEA | |
| CD20 Light | EDAATYYCQQWSFNPPTFGAGTKLELKRTVAAPSVFIFP | |
| chain 1 | PSDEQLKSGTASVVCLLNNFYPREAKVQWKVDNALQS | |
| | GNSQESVTEQDSKDSTYSLSSTLTLSKADYEKHKVYACE | |
| | VTHQGLSSPVTKSFNR | |

Tositumomab binds to the CD20 antigen, which is predominantly expressed on mature B cells and on >90% of B-cell non-Hodgkin's lympohomas. The antibody leads to selective killing of B-cells. Particularly, the Bexxar regimen composing of tositumomab/iodine-131 tositumomab, can delivers radiation, which enhances the killing effect of the antibody. Normal B cells then will recover in 6-9 months because the parent B-cells do not express the CD20 antigen. Multiple mechanisms of action have been proposed for tumor killing by the Bexxar regimen, including the following 1) apoptosis, 2) complement-dependent cytotoxicity, 3) antibody-dependent cellular cytotoxicity, and 4) ionizing radiation from the radioisotope. In addition, a potential vaccine-like effect leading to adaptive immunity against cells that survive initial treatment is also suggested.

Non-Hodgkin's lymphoma (NHL) refers to any of a large group of cancers of lymphocytes (white blood cells). Non-Hodgkin lymphomas can occur at any age and are often marked by lymph nodes that are larger than normal, fever, and weight loss. Non-Hodgkin's lymphoma (NHL) may include mantle cell lymphoma, diffuse large B-cell lymphoma (DLBCL), chronic lymphocytic leukemia (CLL)/small lymphocytic lymphoma (SLL), or follicular lymphoma (FL).

The Bexxar therapeutic regimen is intended as a single course of treatment and is indicated for the treatment of patients with CD20 antigen-expressing relapsed or refractory, low-grade, follicular, or transformed non-Hodgkin lymphoma, including patients with rituximab-refractory non-Hodgkin lymphoma. However, it is not indicated for the initial treatment of patients with CD20-positive non-Hodgkin lymphoma (Kaminski M et al., Cancer Oncol, 2007, 3, 255-262: Kaminski M et al., J Clin Oncol, 2005, 23, 7985-7993; the contents of each of which are incorporated herein by reference in their entirety.)

The most serious adverse reactions observed have been severe and prolonged cytopenias and the sequelae of cytopenias, which included infections (sepsis) and hemorrhage in patients with thrombocytopenia, allergic reactions (bronchospasm and angioedema), secondary leukemia, infections (including pneumonia, bacteremia, septicemia, bronchitis, and skin infections) and myelodysplasia. Less common but severe adverse reactions are pneumonia, pleural effusion, and dehydration.

This observed clinical reversed effect is partially due to the significant depletion of infection-fighting white blood cells, oxygen-carry red blood cells, and clot-forming platelet cells and some immune reaction associated with antibodies infusion. According to the present invention, the polynucleotides encoding the antibody may be linked to signal peptides, targeting peptides, or fused with the nuclei acid for targeted delivery to B lymphocytes, thus reducing the reverse effects in other blood cells. Furthermore, the pharmaceutical compositions of the present invention will reduce antibody-fusion caused immune reaction in a subject.

The polynucleotides encoding the amino acid sequences of tositumomab may be formulated and dosed in a fashion consistent with good medical practice, taking into account the clinical condition of the individual patient (especially the side effects of treatment, the side of delivery, the method of administration, the scheduling of administration, and other factors known to practitioners. In some embodiments, tositumomab may be formulated with other monoclonal antibodies to cell surface antigen(s) including but not limited to CD52 antibodies (e.g. alemtuzumab) and other anti-CD antibodies (for example, CD20, CD22 and CD33), such as rituximab/rituximab, or other therapeutic anti-CD20 antibodies e.g. those described in US patent application publication US20090130089, the content of which is incorporated herein by reference.

In one embodiment, the polynucleotides encoding the antibodies or the functional variants thereof may be formulated through a linking moiety such as those described in US Patent publication US20060222588, the content of which is incorporated by reference in its entirety.

In some embodiments the polynucleotides of the present invention may be used alone or in combination with other anti-CD-20 antibodies to treat CD20 positive cancers and/or other clinical conditions. Such anti-CD 20 antibodies include, but are not limited to, ocrelizuman (2H7.v16, PRO-70769, R-1594), IMMU-106, DXL 625, ocaratuzumab (AME-133), TRU-015, ofatumumab (2F2), Rituximab, 11B8 (as disclosed in WO 2004035607), AT80 (as disclosed in WO2009030368), humanized B-Lyl antibody (a chimeric humanized antibody as disclosed in WO2005044859), obinutuzumab (GA101), HI47 Ig G3 (ECACC, hybridoma), 2C6 IgG1 (as disclosed in WO2004056312), 2F2 IgG1 (HuMax-CD20) (as disclosed in WO2004035607 and WO2005103081), 2H7 (as disclosed in WO2004056312), Veltuzumab, anti-CD20 antibodies as described in U.S. Pat. Nos. 8,465,741; 8,097,713; 8,057,793; 7,879,984; and US patent publication NOs: US20130195846; US20090136516; US20090130089; the contents of each of which are herein incorporated by reference in their entirety.

In one aspect, said anti-CD20 antibodies may be encoded by the polynucleotides of the present invention and co-administered with the polynucleotides encoding tositumomab.

In some embodiments the polynucleotides of the present invention may be used with other immunotherapeutic agents such as alemtuzumab, rituximab, gemtuzumab ozogamicin, and ibritumomab tiuxetan for immunotherapy.

In some embodiments the polynucleotides encoding tositumomab may be used in combination with other chemotherapeutics such as CXCR4 antagonists (e.g. US20110280827); CDK inhibitors, as described in US patent publication NO. US20090036435; human TNF delta and TNF epsilon polypeptides, polynucleotides encoding the TNF polypeptides, as described in U.S. Pat. No. 7,217,788; Neutrokine-alpha and/or Neutrokine-alphaSV polypeptides or the polynucleotides encoding the polypeptides (U.S. Pat. Nos. 8,212,004; 8,211,649); protein kinase PKA and/or PKB modulators (e.g. pyrazole derivatives) (e.g. U.S. Pat. No. 8,541,461); Phosphatidylinositol 3-kinase delta inhibitors (e.g. US patent publication NO: US20130064812); anti-angiogenic agents (e.g. U.S. Pat. No. 8,288,349); protein tyrosine kinase inhibitors (e.g. U.S. Pat. No. 8,293,897); the content of each of which are incorporated by reference in their entirety.

According to the present invention, the tositumomab polynucleotides of the present invention are engineered such that they may comprise one or more coding regions.

The coding regions of the tositumomab polynucleotides may encode any of the regions or portions of the tositumomab antibody. They may also further comprise coding regions not found in the original or parent tositumomab antibody.

The tositumomab polynucleotides of the present invention may be engineered to produce any of the 5 standard classes of immunoglobulins as shown in FIG. 1 using the tositumomab antibody or any of its component parts as a starting molecule.

The tositumomab polynucleotides may also be engineered according to the present invention to produce a variant tositumomab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Trastuzumab Parent Molecule or Antibody

According to the present invention, trastuzumab polynucleotides or constructs and their associated trastuzumab compositions are designed to produce the trastuzumab antibody, a variant or a portion thereof in vivo.

Trastuzumab is marketed under the trade name Herceptin (synonym: Herclon) and is a monoclonal antibody that binds to the human epidermal growth factor receptor 2(HER-2)/neu. HER-2, a receptor with tyrosine kinase activity, is expressed at high levels in some breast cancers and also some other types of cancer. Trastuzumab (Herceptin®) is approved to treat certain types of breast cancer as well as some types of gastric or gastroesophageal junction adenocarcinoma. The mechanism by which trastuzumab acts is not completely understood, but one likely possibility is that it prevents HER-2 from sending growth-promoting signals. Trastuzumab may have other effects as well, such as inducing the immune system to attack cells that express high levels of HER-2/neu.

However, trastuzumab administration can result in sub-clinical and clinical cardiac failure in some patients, particularly in patients who receive Herceptin with anthracycline chemotherapy regimens. Trastuzumab can also cause serious and sometimes fatal infusion reaction and pulmonary toxicity. As with other antibody based protein therapies, trastuzumab has the potential to raise the immunogenicity in patients.

Trastuzumab is a recombinant, humanized monoclonal antibody IgG1 kappa that selectively binds with high affinity in a cell-based assay (Kd=5 nM) to the extracellular domain of the human epidermal growth factor receptor protein Her2 (also called c-erbB2).

Trastuzumab was first developed by Dr. Axel Ullrich and Dr. H. Michael Shepard at UCLA as an monoclonal antibody that specifically binds to the extracellular domain of Her2 receptor and its binding inhibits the growth of tumor cells that overexpress Her2 receptor (see. e.g. U.S. Pat. Nos. 5,720,954; 5,770,195; 5,772,997; 6,165,464; 6,387,371; 6,399,063; the contents of each of which are incorporated herein by reference in their entirety).

Certain sequences encoding trastuzumab fragments, domains or heavy or light chains are given in Table 28. The table is not an exhaustive list and any fragment or portion of the sequence may be encoded in the trastuzumab polynucleotides of the invention.

TABLE 28
Table of Trastuzumab Sequences

| | | SEQ ID |
|---|---|---|
| Description | Sequence | NO. |
| Anti-HER2 | EVQLVESGGGLVQPGGSLRLSCAASGFNIKDTYIH | 126 |
| heavy chain 1 | WVRQAPGKGLEWVARIYPTNGYTRYADSVKGRFT | |
| | ISADTSKNTAYLQMNSLRAEDTAVYYCSRWGGDG | |
| | FYAMDYWGQGTLVTVSSASTKGPSVFPLAPSSKST | |
| | SGGTAALGCLVKDYFPEPVTVSWNSGALTSGVHTF | |
| | PAVLQSSGLYSLSSVVTVPSSSLGTQTYICNVNHKP | |
| | SNTKVDKKVEPPKSCDKTHTCPPCPAPELLGGPSVF | |
| | LFPPKPKDTLMISRTPEVTCVVVDVSHEDPEVKFN | |
| | WYVDGVEVHNAKTKPREEQYNSTYRVVSVLTVLH | |
| | QDWLNGKEYKCKVSNKALPAPIEKTISKAKGQPRE | |
| | PQVYTLPPSRDELTKNQVSLTCLVKGFYPSDIAVE | |
| | WESNGQPENNYKTTPPVLDSDGSFFLYSKLTVDKS | |
| | RWQQGNVFSCSVMHEALHNHYTQKSLSLSPGK | |
| Anti-HER2 light | DIQMTQSPSSLSASVGDRVTITCRASQDVNTAVAW | 127 |
| chain 1 | YQQKPGKAPKLLIYSASFLYSGVPSRFSGSRSGTDF | |
| | TLTISSLQPEDFATYYCQQHYTTPPTFGQGTKVEIK | |
| | RTVAAPSVFIFPPSDEQLKSGTASVVCLLNNFYPRE | |
| | AKVQWKVDNALQSGNSQESVTEQDSKDSTYSLSS | |
| | TLTLSKADYEKHKVYACEVTHQGLSSPVTKSFNRG | |
| | EC | |
| Herceptin (L- | DIQMTQSPSSLSASVGDRVTITCRASQDVNTAVAW | 128 |
| KAPPA (V- | YQQKPGKAPKLLIYSASFLYSGVPSRFSGSRSGTDF | |
| KAPPA(1-107) + | TLTISSLQPEDFATYYCQQHYTTPPTFGQGTKVEIK | |
| C- | RTVAAPSVFIFPPSDEQLKSGTASVVCLLNNFYPRE | |
| KAPPA(108-214)) | AKVQWKVDNALQSGNSQESVTEQDSKDSTYSLSS | |
| | TLTLSKADYEKHKVYACEVTHQGLSSPVTKSFNRG | |
| | EC | |
| Herceptin (VH- | EVQLVESGGGLVQPGGSLRLSCAASGFNIKDTYIH | 129 |
| CH1 (VH(1-120) + | WVRQAPGKGLEWVARIYPTNGYTRYADSVKGRFT | |
| CH1(121-218)) | ISADTSKNTAYLQMNSLRAEDTAVYYCSRWGGDG | |
| | FYAMDYWGQGTLVTVSSASTKGPSVFPLAPSSKST | |
| | SGGTAALGCLVKDYFPEPVTVSWNSGALTSGVHTF | |
| | PAVLQSSGLYSLSSVVTVPSSSLGTQTYICNVNHKP | |
| | SNTKVDKKVEP | |
| Trastuzumab | EVQLVESGGGLVQPGGSLRLSCAASGFNIKDTYIH | 130 |
| (H-GAMMA-1 | WVRQAPGKGLEWVARIYPTNGYTRYADSVKGRFT | |
| (VH(1-120) + | ISADTSKNTAYLQMNSLRAEDTAVYYCSRWGGDG | |
| CH1(121-218) + | FYAMDYWGQGTLVTVSSASTKGPSVFPLAPSSKST | |
| HINGE- | SGGTAALGCLVKDYFPEPVTVSWNSGALTSGVHTF | |
| REGION(219-233) + | PAVLQSSGLYSLSSVVTVPSSSLGTQTYICNVNHKP | |
| CH2(234-343) + | SNTKVDKKVEPKSCDTPPPCPRCPAPELLGGPSVFL | |
| CH3(344-450) | FPPKPKDTLMISRTPEVTCVVVDVSHEDPEVKFNW | |
| | YVDGVEVHNAKTKPREEQYNSTYRVVSVLTVLHQ | |
| | DWLNGKEYKCKVSNKALPAPIEKTISKAKGQPREP | |
| | QVYTLPPSRDELTKNQVSLTCLVKGFYPSDIAVEW | |
| | ESNGQPENNYKTTPPVLDSDGSFFLYSKLTVDKSR | |
| | WQQGNVFSCSVMHEALHNHYTQKSLSLSPGK | |
| Trastuzumab (L- | DIQMTQSPSSLSASVGDRVTITCRASQDVNTAVAW | 131 |
| KAPPA (V- | YQQKPGKAPKLLIYSASFLYSGVPSRFSGSRSGTDF | |
| KAPPA(1-107) + | TLTISSLQPEDFATYYCQQHYTTPPTFGQGTKVEIK | |
| C- | RTVAAPSVFIFPPSDEQLKSGTASVVCLLNNFYPRE | |
| KAPPA(108-214)) | AKVQWKVDNALQSGNSQESVTEQDSKDSTYSLSS | |
| | TLTLSKADYEKHKVYACEVTHQGLSSPVTKSFNRG | |
| | EC | |
| Trastuzumab | DIQMTQSPSSLSASVGDRVTITCRASQDVNTAVAW | 132 |
| light chain | YQQKPG | |
| variable region | KAPKLLIYSASFLYSGVPSRFSGSRSGTDFTLTISSL | |
| (VL) | QPEDFATYYCQQHYTTPPTFGQGTKVEIKRT | |
| From | | |
| U.S. Pat. No. 8,557,243 | | |
| VL-CDR1 | RASQDVNTAVAW | 133 |
| From | | |
| U.S. Pat. No. 8,557,243 | | |
| VL-CDR2 | SASFLYS | 134 |
| From | | |
| U.S. Pat. No. 8,557,243 | | |
| VL-CDR3 | QQHYTTPPT | 135 |
| From | | |
| U.S. Pat. No. 8,557,243 | | |
| Trastuzuman | VQLVESGGGLVQPGGSLRLSCAASGFNIKDTYIHW | 136 |
| heavy chain | VRQAPGKGLEWVARIYPTNGYTRYADSVKGRFTIS | |
| variable regions | ADTSKNTAYLQMNSLRAEDTAVYYCSRWGGDGF | |
| (VH) | YAMDYWGQGTLVTVSS | |
| From | | |
| U.S. Pat. No. 8,557,243 | | |
| VH-CDR1 | GRNIKDTYIH | 137 |
| From | | |
| U.S. Pat. No. 8,557,243 | | |
| VH-CDR2 | RIYPTNGYTRYADSVKG | 138 |
| From | | |
| U.S. Pat. No. 8,557,243 | | |
| VH-CDR3 | WGGDGFYAMDY | 139 |
| From | | |
| U.S. Pat. No. 8,557,243 | | |

Trastuzumab specifically binds to Her-2/neu, a member o the HER family receptor tyrosine kinases. The Her receptors are cell surface molecules and the binding of their ligands (e.g. EGF) can activate Her receptors and tansport signals from outside the cell in inside the cell. These signals stimulate cell proliferation. The overexpression Her receptors can cause cancers, for example, her2 overexpressed in certain types of breast cancer.

Trastuzumab (Herceptin) has been shown, in both in vitro assays and in animals, to inhibit the proliferation of human tumor cells that overexpree Her2. Trastuzumab is a mediator of antibody dependent cellular cytotoxicity (ADCC). In vitro, trastuzumab mediated ADCC has been shown to be preferentially exerted on Her2 overexpressed cancer cells as compared with cancer cells that do not overexpress Her2.

Trastuzumab is indicated for the treatment of early stage Her2 positive breast cancer, or metastatic breast cancer as well as other cancers. Trastuzumab (Herceptin) is indicated for adjuvant treatment of HER2 overexpressing node positive or node negative (ER/PR negative or with one high risk feature breast cancer, either as part of a treatment regimen consisting of doxorubicin, cyclophosphamide, and either paclitaxel or docetaxel, or with docetaxel and carboplatin, or as a single agent following multi-modality anthracycline based therapy. Trastuzumab (Herceptin) is indicated, in combination with paclitaxel for first-line treatment of ER2-overexpressing metastatic breast cancer. It may also be used as a single agent for treatment of HER2-overexpressing breast cancer in patients who have received one or more chemotherapy regimens for metastatic disease.

Furthermore, Trastuzumab (Herceptin) is indicated, in combination with cisplatin and capecitabine or 5-fluorouracil, for the treatment of patients with HER2 overexpressing metastatic gastric or gastroesophageal junction adenocarcinoma, who have not received prior treatment for metastatic disease.

In some embodiments, the polynucleotides encoding trastuzumab may be used as targeted therapy agent in cancers, particularly in those with overexpressed Her-2 receptor.

The polynucleotides encoding trastuzumab may be used to treat adjuvant treatment of HER2 overexpressing node positive or node negative (ER/PR negative or with one high risk feature breast cancer, either as part of a treatment regimen consisting of doxorubicin, cyclophosphamide, and either paclitaxel or docetaxel, or with docetaxel and carboplatin, or as a single agent following multi-modality anthracycline based therapy.

The polynucleotides encoding trastuzumab may be used, in combination with paclitaxel for first-line treatment of ER2-overexpressing metastatic breast cancer. It may also be used as a single agent for treatment of HER2-overexpressing breast cancer in patients who have received one or more chemotherapy regimens for metastatic disease.

The polynucleotides encoding trastuzumab may be used, in combination with cisplatin and capecitabine or 5-fluorouracil, for the treatment of patients with HER2 overexpressing metastatic gastric or gastroesophageal junction adenocarcinoma, who have not received prior treatment for metastatic disease

When used for in vivo therapy, the polynucleotides encoding trastuzumab may be administered to the patient to generate the therapeutically effective amounts of antibodies (i.e. amounts that eliminate or reduce the patient's tumor burden).

According to the present invention, the trastuzumab polynucleotides of the present invention are engineered such that they may comprise one or more coding regions.

The coding regions of the trastuzumab polynucleotides may encode any of the regions or portions of the trastuzumab antibody. They may also further comprise coding regions not found in the original or parent trastuzumab antibody.

The trastuzumab polynucleotides of the present invention may be engineered to produce any of the 5 standard classes of immunoglobulins as shown in FIG. 1 using the trastuzumab antibody or any of its component parts as a starting molecule.

The trastuzumab polynucleotides may also be engineered according to the present invention to produce a variant trastuzumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Catumaxomab Parent Molecule or Antibody

According to the present invention, catumaxomab polynucleotides or constructs and their associated catumaxomab compositions are designed to produce the catumaxomab antibody, a variant or a portion thereof in vivo.

Catumaxomab, also known as REMOVAB® or anti-EpCAM antibody, is a monoclonal bispecific (anti-EpCAM×anti-CD3) trifunctional antibody. Catumaxomab was developed by Fresenius Biotech together with Trion Pharma to contain three different binding sites, (1) a mouse Fab fragment that binds to human EpCAM, (2) a rat Fab region that binds to human CD3 and (3) a hybrid Fc region that permits the binding of Fc-gamma receptor. Catumaxomab was approved by the European Union in April 2009 to treat patients with malignant ascites due to epithelial carcinomas.

In one embodiment, the polynucleotides described herein encode a monoclonal bispecific trifunctional antibody that can bind to three different binding sites, (1) a mouse Fab fragment that binds to human EpCAM, (2) a rat Fab region that binds to human CD3 and (3) a hybrid Fc region that permits the binding of Fc-gamma receptor. These polynucleotides may bind to and activate Fc-gamma receptor I-, IIa- or III-positive accessory cells but may not bind to inhibitory Fc-gamma receptor type IIb accessory cells. As a non-limiting example, the polynucleotides can encode catumaxomab or a fragment or variant thereof.

In one embodiment, the polynucleotides described herein encode a modulator of a target, such as, but not limited to, epithelial cell adhesion molecule (EpCAM). As a non-limiting example, the polynucleotide can encode catumaxomab or a fragment or variant thereof.

In one embodiment, the polynucleotides described herein encode a modulator of a target, such as, but not limited to, CD3. As a non-limiting example, the polynucleotide can encode catumaxomab or a fragment or variant thereof.

In one embodiment, the polynucleotides described herein encode a modulator of a target, such as, but not limited to, Fc-gamma receptor. As a non-limiting example, the polynucleotide can encode catumaxomab or a fragment or variant thereof.

In one embodiment, the polynucleotides described herein encode an antibody which can destroy EpCAM positive tumor cells in the peritoneal cavity. As a non-limiting example, the polynucleotide can encode caumaxomab or a fragment or variant thereof.

In one embodiment, the polynucleotides described herein may encode catumaxomab or a fragment or variant thereof. These polynucleotides may be used to treat a variety of diseases and/or disorders such as but not limited to, malignant ascites, peritoneal carcinomatosis, ovarian cancer and gastric cancer (see Linke et al. mAbs 2010. 2(2), 129-136; the contents of which are herein incorporated by reference in its entirety).

In one embodiment, the polynucleotides described herein may encode catumaxomab or a fragment or variant thereof and may be used as a treatment in those with EpCAM-positive carcinomas. EpCAM-positive carcinomas are cancers where the tumor cells have large quantities of the molecule EpCAM on their surface.

In one embodiment, the polynucleotides described herein may encode catumaxomab or a fragment or variant thereof and may be used to treat malignant ascites. As a non-limiting example, the treatment of a subject with catumaxomab may be after the standard treatment known in the art is not available or is no longer feasible.

In one embodiment, the polynucleotides described herein may encode catumaxomab or a fragment or variant thereof and may be used to treat epithelial carcinomas.

Certain sequences encoding catumaxomab fragments, domains or heavy or light chains are described in Table 29. The table is not an exhaustive list and any fragment or portion of the sequence may be encoded in the catumaxomab polynucleotides of the invention.

TABLE 29
Table of Catumaxomab Related Sequences

| | | SEQ |
|---|---|---|
| Descrip- | | ID |
| tion | Sequence | NO. |
| Chain A, | GGPSVFLFPPKPKDTLMISRTPEVTCVVVDVSHEDPEVK | 140 |
| Human | FNWYVDGVEVHNAKTKPREEQYNSTYRVVSVLTVLHQ | |
| Igg1 | DWLNGKEYKCKVSNKALPAPIEKTISKAKGQPREPQVY | |
| Fc | | |
| Fragment | TLPPSRDELTKNQVSLTCLEVKGFYPSDIAVEWESNGQP | |
| | ENNYKTTPPVLDSDGSFFLYSKRLTVDKSRWQQGNVFS | |
| | CSVMHEALHNHYTQKSLSL | |
| Chain B, | GGPSVFLFPPKPKDTLMISRTPEVTCVVVDVSHEDPEVK | 141 |
| Human | FNWYVDGVEVHNAKTKPREEQYNSTYRVVSVLTVLHQ | |
| Igg1 | DWLNGKEYKCKVSNKALPAPIEKTISKAKGQPREPQVY | |
| Fc | | |
| Fragment | TLPPSRDELTKNQVSLTCLEVKGFYPSDIAVEWESNGQP | |
| | ENNYKTTPPVLDSDGSFFLYSKRLTVDKSRWQQGNVFS | |
| | CSVMHEALHNHYTQKSLSLS | |

The polynucleotides of the invention may encode an amino acid sequence of at least one of the antibodies described in Zeidler et al., British Journal of Cancer, vol 83(2), 261-266 (2000), Lindhofer et al., British Journal of Cancer, vol 97, 315-321 (2007), or Lindhofer et al., mAbs, vol. 2(3), 309-319 (2010), the contents of each of which are incorporated herein by reference in their entirety, wherein the antibody is bispecific and comprises a rat IgG2b that binds to CD3, a mouse IgG2a that binds to EpCAM, and an Fc region that binds and activates Fcγreceptor positive accessory cells.

In one embodiment, the rat IgG2b that binds to CD3 is 26116 and the mouse IgG2a that binds to EpCAM is C215 described in Zeidler et al., British Journal of Cancer, vol 83(2), 261-266 (2000), the contents of which are incorporated herein by reference in their entirety.

In one embodiment, the polynucleotides of the invention may encode an amino acid sequence of at least one of the antibodies described in U.S. Pat. No. 8,277,806 to Lindhofer or US 2010/0322933 to Lindhofer et al., the contents of each of which are incorporated herein by reference in their entirety, wherein the antibody is bispecific and comprises a rat IgG2b that binds to CD3, a mouse IgG2a that binds to EpCAM, and an Fc portion that binds to an Fc receptor-positive cell in the subject that comprises an Fcγreceptor 1, an Fcγreceptor III, or a combination thereof.

In another embodiment, the mouse IgG2a that binds to EpCAM is HO-3 described in Lindhofer et al., British Journal of Cancer, vol 97, 315-321 (2007), the contents of which are incorporated herein by reference in their entirety.

In one embodiment, the polynucleotides described herein may encode catumaxomab or a fragment or variant thereof and may not cause a side-effect, such as, but not limited to, fever, nausea, vomiting and abdominal pain.

In one embodiment, the polynucleotides described herein may encode Catumaxomab or a fragment or variant thereof and may not cause a side-effect such as, but not limited to, abdominal pain, feeling sick (nausea), vomiting and diarrhea, fever and chills, tiredness, loss of appetite, dehydration, a very fast heart beat, high or low blood pressure, abdominal pain accompanied by difficulty passing stools, constipation, shortness of breath, accumulation of fluid around the lungs which cause chest pain and breathlessness, low blood oxygen levels, inflammation of the bile ducts, skin redness, rash, severe allergic skin reaction (dermatitis), very fast heart beat, fever, shortness of breath, feeling faint or light-headed, complex of reactions due to the release of mediators of inflammation, lumps under the skin on the back of the legs that may become sores and leave scars, inflammation and pain or burning and stinging in the area around the catheter, reduction in number of blood platelets, blockage in the gut or bowel, bleeding in the stomach or gut, shown by the vomiting of blood or the passage of red or black stools, skin reaction, fits, lung problems including blood clot in the lungs, severe kidney problems and worsening of general state of health.

In one embodiment, the polynucleotides described herein encoding catumaxomab or a fragment or variant thereof are formulated for intraperitoneal administration. As a non-limiting example, the polynucleotides may be administered as 3 hour infusions.

In one embodiment, the polynucleotides described herein encoding catumaxomab or a fragment or variant thereof are formulated for intravenous administration.

In one embodiment, the polynucleotides encoding Catumaxomab or a fragment or variant thereof may be formulated at a strength of 10 ug/0.1 mL or 50 ug/0.5 mL.

In one embodiment, the polynucleotides encoding catumaxomab or a fragment or variant thereof has a half-life of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 day or at least 14 days.

In one embodiment, the dose of the polynucleotides encoding catumaxomab or a fragment or variant thereof may be between 0.001 and 100 mg, including, but not limited to, 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg and 100 mg. As a non-limiting example, the dose of the polynucleotides encoding catumaxomab or a fragment or variant thereof may be 0.01 mg, 0.02 mg, 0.05 mg, 0.15 mg or 0.2 mg.

In one embodiment, the polynucleotides described herein encoding catumaxomab or a fragment or variant thereof are administered after a subject has undergone at least one round of chemotherapy.

In one embodiment, the polynucleotides described herein encoding catumaxomab or a fragment or variant thereof are administered after a premedication of paracetamol. As a non-limiting example, the premedication of paracetamol may be 1,000 mg.

In one embodiment, the polynucleotides described herein encoding catumaxomab or a fragment or variant thereof are administered as four 6 hour intraperitoneal administrations on days 0, 3, 7 and 10 at escalating doses of 0.01 mg, 0.02 mg, 0.05 mg and 0.15 mg.

In one embodiment, the polynucleotides described herein encoding catumaxomab or a fragment or variant thereof are administered four times in an escalating dose scheme of 0.01 mg, 0.02 mg, 0.05 mg and 0.1 mg.

In one embodiment, the polynucleotides described herein encoding catumaxomab or a fragment or variant thereof may be used to treat malignant ascites due to ovarian cancer. As a non-limiting example, after a premedication of 1,000 mg of paracetamol an escalating dosing scheme of 0.01 mg, 0.02 mg, 0.05 mg and 0.2 mg is administered by 6 hour intraperitoneal administration by escalating doses on days 0, 3, 7 and 10.

In one embodiment, the polynucleotides described herein encoding catumaxomab or a fragment or variant thereof may be used to platinum-refractory epithelial ovarian cancer. As a non-limiting example, an escalating dosing scheme of 0.01 mg, 0.02 mg, 0.05 mg and 0.1 mg is administered by intraperitoneal administration.

In one embodiment, the polynucleotides described herein encoding catumaxomab or a fragment or variant thereof may be delivered by intrapleural administration. The polynucleotides may be used to treat malignant pleural effusion. As a non-limiting example, the polynucleotides encoding Catumaxomab or a fragment or variant thereof may be administered in three escalating doses of 0.01 mg, 0.02 mg and 0.05 mg.

In one embodiment, the polynucleotides described herein encoding catumaxomab or a fragment or variant thereof may be delivered by intravenous administration for the treatment of non-small cell lung cancer. As a non-limiting example, the polynucleotides encoding catumaxomab or a fragment or variant thereof may be administered in escalating doses of 0.002 mg, 0.005 mg and 0.0075 mg as a single intravenous infusion. The polynucleotides encoding catumaxomab may be administered with dexamethasone as a premedication.

According to the present invention, the catumaxomab polynucleotides of the present invention are engineered such that they may comprise one or more coding regions.

The coding regions of the catumaxomab polynucleotides may encode any of the regions or portions of the catumaxomab antibody. They may also further comprise coding regions not found in the original or parent catumaxomab antibody.

The catumaxomab polynucleotides of the present invention may be engineered to produce any of the 5 standard classes of immunoglobulins as shown in FIG. 1 using the catumaxomab antibody or any of its component parts as a starting molecule.

The catumaxomab polynucleotides may also be engineered according to the present invention to produce a variant catumaxomab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Ustekinumab Parent Molecule or Antibody

According to the present invention, ustekinumab polynucleotides or constructs and their associated ustekinumab compositions are designed to produce the ustekinumab antibody, a variant or a portion thereof in vivo.

Ustekinumab, also known as STELARA®, is a fully human IgG1 kappa monoclonal antibody developed in transgenic mice in which the mouse immunoglobulin genes have been inactivated and replaced with human immunoglobulin chains. Ustekinumab is marketed for the treatment of psoriasis and psoriatic arthritis as ustekinumab is an immunosuppressant that reduces the effects of a chemical substance in the body that can cause inflammation.

In one embodiment, the polynucleotides described herein encode a fully human IgG1 kappa monoclonal antibody which has been designed to attach to interleukin 12 and interleukin 23. As a non-limiting example, the polynucleotides can encode ustekinumab or a fragment or variant thereof.

In one embodiment, the polynucleotides described herein encode a fully human IgG1 kappa monoclonal antibody which has been designed to block p40 binding to Interleukin 12 and interleukin 23 receptors. As a non-limiting example, the polynucleotides can encode ustekinumab or a fragment or variant thereof.

In one embodiment, the polynucleotides described herein encode a fully human IgG1 kappa monoclonal antibody which has been designed to inhibit interleukin 12 and interleukin 23 signaling. As a non-limiting example, the polynucleotides can encode ustekinumab or a fragment or variant thereof.

In one embodiment, the polynucleotides described herein encode a modulator of a target, such as, but not limited to, p40 binding to interleukin 12 and interleukin 23. As a non-limiting example, the polynucleotide can encode ustekinumab or a fragment or variant thereof.

In one embodiment, the polynucleotides described herein encode a modulator of a target, such as, but not limited to, inhibiting interleukin 12 signaling. As a non-limiting example, the polynucleotide can encode ustekinumab or a fragment or variant thereof.

In one embodiment, the polynucleotides described herein encode a modulator of a target, such as, but not limited to, inhibiting interleukin 23 signaling. As a non-limiting example, the polynucleotide can encode ustekinumab or a fragment or variant thereof.

In one embodiment, polynucleotides may be used to treat a variety of diseases and/or disorders such as but not limited to, plaque psoriasis and active psoriatic arthritis. As a non-limiting example, the disease and/or disorder may be severe plaque psoriasis.

In one embodiment, the polynucleotides described herein may be used to treat adults (18 years and older) who may suffer from moderate to severe plaque psoriasis.

As a non-limiting example, the polynucleotides described herein may be used as a treatment option for those who failed to respond or cannot use systemic treatments for psoriasis. The polynucleotides may be used as a treatment either alone or in combination with methotrexate therapy.

In one embodiment, the polynucleotides described herein may encode ustekinumab or a fragment or variant thereof and may be used to treat adults (18 years and older) who suffer from active psoriatic arthritis. The polynucleotides may be used as a treatment in those who have not responded to other treatments using disease-modifying antirheumatic drugs (DMARDs). In addition, the polynucleotides may be used as a treatment either alone or in combination with methotrexate therapy.

Certain sequences encoding ustekinumab fragments, domains or heavy or light chains are given in Table 30. The table is not an exhaustive list and any fragment or portion of the sequence may be encoded in the ustekinumab polynucleotides of the invention.

TABLE 30
Table of Ustekinumab Sequences

| | | SEQ ID |
|---|---|---|
| Description | Sequence | NO |
| CDR1 Heavy Chain (See Int'l Patent Pub | TYWLG | 142 |
| WO2014004436 SEQ ID NO: 16 and U.S. Pat. | | |
| No. 6.902,734 SEQ ID NO: 1; the contents of | | |
| each of which are herein incorporated by | | |
| reference in their entirety) | | |
| CDR2 Heavy Chain (See Int'l Patent Pub | IMSPVDSDIRYSPSFQ | 143 |
| WO2014004436 SEQ ID NO: 17 and U.S. Pat. | | |
| No. 6.902,734 SEQ ID NO: 2; the contents of | | |
| each of which are herein incorporated by | | |
| reference in their entirety) | | |
| CDR3 Heavy Chain (See Int'l Patent Pub | RRPGQGYFDF | 144 |
| WO2014004436 SEQ ID NO: 18 and U.S. Pat. | | |
| No. 6.902,734 SEQ ID NO: 3; the contents of | | |
| each of which are herein incorporated by | | |
| reference in their entirety) | | |
| CDR1 Light Chain (See Int'l Patent Pub | RASQGISSWLA | 145 |
| WO2014004436 SEQ ID NO: 19 and U.S. Pat. | | |
| No. 6.902,734 SEQ ID NO: 4; the contents of | | |
| each of which are herein incorporated by | | |
| reference in their entirety) | | |
| CDR2 Light Chain (See Int'l Patent Pub | AASSLQS | 146 |
| WO2014004436 SEQ ID NO: 20 and U.S. Pat. | | |
| No. 6.902,734 SEQ ID NO: 5; the contents of | | |
| each of which are herein incorporated by | | |
| reference in their entirety) | | |
| CDR3 Light Chain (See Int'l Patent Pub | QQYNIYPYT | 147 |
| WO2014004436 SEQ ID NO: 21 and U.S. Pat. | | |
| No. 6.902,734 SEQ ID NO: 6; the contents of | | |
| each of which are herein incorporated by | | |
| reference in their entirety) | | |
| Heavy Chain Variable Domain (See Int'l Patent | EVQLVQSGAEVKKPGE | 148 |
| Pub WO2014004436 SEQ ID NO: 22 and U.S. | SLKISCKGSGYSFTTYW | |
| Pat. No. 6.902,734 SEQ ID NO: 7; the contents | LGWVRQMPGKGLDWI | |
| of each of which are herein incorporated by | GIMSPVDSDIRYSPSFQG | |
| reference in their entirety) | QVTMSVDKSITTAYLQ | |
| | WNSLKASDTAMYYCAR | |
| | RRPGQGYFDFWGQGTL | |
| | VTVSS | |
| Light Chain Variable Domain (See Int'l Patent | DIQMTQSPSSLSASVGD | 149 |
| Pub WO2014004436 SEQ ID NO: 23 and U.S. | RVTITCRASQGISSWLA | |
| Pat. No. 6.902,734 SEQ ID NO: 8; the contents | WYQQKPEKAPKSLIYA | |
| of each of which are herein incorporated by | ASSLQSGVPSRFSGSGS | |
| reference in their entirety) | GTDFTLTISSLQPEDFAT | |
| | YYCQQYNIYPYTFGQGT | |
| | KLEIKR | |
| Heavy Chain (See Int'l Patent Pub | EVQLVQSGAEVKKPGE | 150 |
| WO2014004436 and SEQ ID NO: 24; the | SLKISCKGSGYSFTTYW | |
| contents of which are herein incorporated by | LGWVRQMPGKGLDWI | |
| reference in their entirety) | GIMSPVDSDIRYSPSFQG | |
| | QVTMSVDKSITTAYLQ | |
| | WNSLKASDTAMYYCAR | |
| | RRPGQGYFDFWGQGTL | |
| | VTVSSSSTKGPSVFPLAP | |
| | SSKSTSGGTAALGCLVK | |
| | DYFPEPVTVSWNSGALT | |
| | SGVHTFPAVLQSSGLYS | |
| | LSSVVTVPSSSLGTQTYI | |
| | CNVNHKPSNTKVDKRV | |
| | EPKSCDKTHTCPPCPAP | |
| | ELLGGPSVFLFPPKPKDT | |
| | LMISRTPEVTCVVVDVS | |
| | HEDPEVKFNWYVDGVE | |
| | VHNAKTKPREEQYNST | |
| | YRVVSVLTVLHQDWLN | |
| | GKEYKCKVSNKALPAPI | |
| | EKTISKAKGQPREPQVY | |
| | TLPPSRDELTKNQVSLT | |
| | CLVKGFYPSDIAVEWES | |
| | NGQPENNYKTTPPVLDS | |
| | DGSFFLYSKLTVDKSRW | |
| | QQGNVFSCSVMHEALH | |
| | NHYTQKSLSLSPGK | |
| Light Chain (See Int'l Patent Pub | DIQMTQSPSSLSASVGD | 151 |
| WO2014004436 and SEQ ID NO: 25; the | RVTITCRASQGISSWLA | |
| contents of which are herein incorporated by | WYQQKPEKAPKSLIYA | |
| reference in their entirety) | ASSLQSGVPSRFSGSGS | |
| | GTDFTLTISSLQPEDFAT | |
| | YYCQQYNIYPYTFGQGT | |
| | KLEIKRTVAAPSVFIFPP | |
| | SDEQLKSGTASVVCLLN | |
| | NFYPREAKVQWKVDNA | |
| | LQSGNSQESVTEQDSKD | |
| | STYSLSSTLTLSKADYE | |
| | KHKVYACEVTHQGLSS | |
| | PVTKSFNRGEC | |

In one embodiment, the polynucleotides described herein may encode ustekinumab or a fragment or variant thereof and may not cause a side-effect, such as, but not limited to, serious infections, cancers and reversible posterior leukoencephalopathy syndrome (RPLS).

In one embodiment, the polynucleotides described herein may encode ustekinumab or a fragment or variant thereof and may not cause a serious infection side-effect such as, but not limited to, those which may require hospitalization such as tuberculosis.

In one embodiment, the polynucleotides described herein may encode ustekinumab or a fragment or variant thereof and may not cause a side-effect such as an infectious disease most commonly of the lungs resulting in coughing, fever, chest pain and weight loss.

In one embodiment, the polynucleotides described herein may encode ustekinumab or a fragment or variant thereof and may not cause a side-effect such as an infection caused by bacteria, fungi or viruses.

In one embodiment, the polynucleotides described herein may encode ustekinumab or a fragment or variant thereof and may not increase your risk for certain types of cancer. As a non-limiting example, the polynucleotides described herein will not cause skin cancer.

In one embodiment, the polynucleotides described herein may encode ustekinumab or a fragment or variant thereof and may not cause reversible posterior leukoencephalopathy syndrome (RPLS). As a non-limiting example, the polynucleotides do not cause RPLS or any of the symptoms of RPLS including headache, seizures, confusion, and vision problems.

In one embodiment, the polynucleotides described herein may encode ustekinumab or a fragment or variant thereof and may not cause any of the side effects associated with STELARA® such as, but not limited to, cold symptoms, headache, fever, chills, muscle pain, shortness of breath, weight loss, diarrhea or stomach pain, burning when you urinate, mild tiredness, feeling very tired, skin warmth or redness, painful skin sores, or coughing up blood.

In one embodiment, the polynucleotides described herein encoding ustekinumab or a fragment or variant thereof may be administered with a blood thinner such as, but not limited to, warfarin or Coumadin without causing adverse effects.

In one embodiment, the polynucleotides described herein encoding ustekinumab or a fragment or variant thereof may be administered with drugs that are known to weaken the immune system (e.g., cancer medicine or steroids) without causing adverse effects.

In one embodiment, the polynucleotides described herein encoding Ustekinumab or a fragment or variant thereof may be administered with drugs that are known to prevent organ transplant rejection (e.g., cyclosporine, sirolimus or tacrolimus) without causing adverse effects.

In one embodiment, the polynucleotides encoding ustekinumab or a fragment or variant thereof has a half-life of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 day or at least 14 days.

In one embodiment, the dose of the polynucleotides encoding ustekinumab or a fragment or variant thereof may be between 0.001 and 100 mg, including, but not limited to, 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg and 100 mg. As a non-limiting example, the dose may be 45 mg or 90 mg. As another non-limiting example, a subject weighing less than 100 kg may receive a dose of 45 mg and a subject weighing more than 100 kg may receive a dose of 90 mg.

In one embodiment, the polynucleotides encoding ustekinumab or a fragment or variant thereof may be formulated with inactive ingredients such as, but not limited to, L-histidine, L-histidine monohydrochloride monohydrate, polysorbate 80, and sucrose.

In one embodiment, the polynucleotides encoding ustekinumab or a fragment or variant thereof may be administered at week 0, week 4 and every 12 weeks after week 4. The subject may receive an injection of 45 mg or 90 mg of the ustekinumab polynucleotides at each of the administrations.

According to the present invention, the ustekinumab polynucleotides of the present invention are engineered such that they may comprise one or more coding regions.

The coding regions of the ustekinumab polynucleotides may encode any of the regions or portions of the ustekinumab antibody. They may also further comprise coding regions not found in the original or parent ustekinumab antibody.

The ustekinumab polynucleotides of the present invention may be engineered to produce any of the 5 standard classes of immunoglobulins as shown in FIG. 1 using the ustekinumab antibody or any of its component parts as a starting molecule.

The ustekinumab polynucleotides may also be engineered according to the present invention to produce a variant ustekinumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Pertuzumab Parent Molecule or Antibody

According to the present invention, pertuzumab polynucleotides or constructs and their associated pertuzumab compositions are designed to produce the pertuzumab antibody, a variant or a portion thereof in vivo.

Pertuzumab, also known as PERJECTA®, 2C4 Antibody, MOAB 2C4, Monoclonal Antibody 2C4, Omnitarg, R1273 and rhuMAb-2C4 is a recombinant humanized monoclonal antibody. Pertuzumab is approved by the FDA as a treatment for HER2-positive metastatic breast cancer. Pertuzumab may be used as a targeted therapy in those with HER2-positive metastatic breast cancer in combination with Herceptin (trastuzumab) and/or Docetaxel (chemotherapy).

In one embodiment, the polynucleotides described herein encode a recombinant humanized monoclonal antibody that targets the extracellular dimerization domain (Subdomain II) of the human epidermal growth factor receptor 2 protein (HER2). As a non-limiting example, the polynucleotides can encode pertuzumab or a fragment or variant thereof.

In one embodiment, the polynucleotides described herein encode a modulator of a target, such as, but not limited to, HER2. As a non-limiting example, the polynucleotide can encode pertuzumab or a fragment or variant thereof.

In one embodiment, the polynucleotides described herein encode a humanized monoclonal antibody that binds to the HER2 receptor and can inhibit the ability of HER2 to interact with other HER family members including, but not limited to, HER1, HER3 and HER4 on the surface of cancer cells. As a non-limiting example, the polynucleotide can encode pertuzumab or a fragment or variant thereof.

In one embodiment, the polynucleotides described herein encode a monoclonal antibody that targets the extracellular dimerization domain (Subdomain II) of the human epidermal growth factor receptor 2 protein (HER2). As a non-limiting example, the polynucleotide can encode pertuzumab or a fragment or variant thereof.

In one embodiment, the polynucleotides described herein encode a monoclonal antibody that targets receptor tyrosine-protein kinase erbB-2. As a non-limiting example, the polynucleotide can encode pertuzumab or a fragment or variant thereof.

In one embodiment, the polynucleotides described herein may encode pertuzumab or a fragment or variant thereof. These polynucleotides may be used to treat a variety of diseases and/or disorders such as but not limited to, breast cancer. As a non-limiting example, the breast cancer may be HER2-positive metastatic breast cancer.

In one embodiment, the polynucleotides described herein may encode pertuzumab or a fragment or variant thereof and may be used to treat early stage breast cancer. The polynucleotides may be used alone or as part of a complete treatment regimen for those with early stage breast cancer. As a non-limiting example, the polynucleotides described herein may be used for treatment prior to the subject undergoing surgery related to breast cancer.

In one embodiment, the polynucleotides described herein may encode pertuzumab or a fragment or variant thereof and may be used to treat advanced or late-stage (metastatic) HER2-positive breast cancer.

Certain sequences encoding pertuzumab fragments, domains or heavy or light chains are given in Table 31. The table is not an exhaustive list and any fragment or portion of the sequence may be encoded in the pertuzumab polynucleotides of the invention.

TABLE 31
Table of Pertuzumab Sequences

| | | SEQ |
|---|---|---|
| | | ID |
| Description | Sequence | NO |
| Heavy Chain | EVQLVESGGGLVQPGGSLRLSCAASGFTFTDYTMD | 152 |
| | WVRQAPGKGLEWVADVNPNSGGSIYNQRFKGRFT | |
| | LSVDRSKNTLYLQMNSLRAEDTAVYYCARNLGPSF | |
| | YFDYWGQGTLVTVSSASTKGPSVFPLAPSSKSTSG | |
| | GTAALGCLVKDYFPEPVTVSWNSGALTSGVHTFPA | |
| | VLQSSGLYSLSSVVTVPSSSLGTQTYICNVNHKPSN | |
| | TKVDKKVEPKSCDKTHTCPPCPAPELLGGPSVFLFP | |
| | PKPKDTLMISRTPEVTCVVVDVSHEDPEVKFNWYV | |
| | DGVEVHNAKTKPREEQYNSTYRVVSVLTVLHQDW | |
| | LNGKEYKCKVSNKALPAPIEKTISKAKGQPREPQV | |
| | YTLPPSREEMTKNQVSLTCLVKGFYPSDIAVEWES | |
| | NGQPENNYKTTPPVLDSDGSFFLYSKLTVDKSRWQ | |
| | QGNVFSCSVMHEALHNHYTQKSLSLSPG | |
| Light Chain | DIQMTQSPSSLSASVGDRVTITCKASQDVSIGVAWY | 153 |
| | QQKPGKAPKLLIYSASYRYTGVPSRFSGSGSGTDFT | |
| | LTISSLQPEDFATYYCQQYYIYPYTFGQGTKVEIKR | |
| | TVAAPSVFIFPPSDEQLKSGTASVVCLLNNFYPREA | |
| | KVQWKVDNALQSGNSQESVTEQDSKDSTYSLSSTL | |
| | TLSKADYEKHKVYACEVTHQGLSSPVTKSFNRGEC | |

In one embodiment, the polynucleotides described herein may encode the variable light and/or variable heavy domains of pertuzumab as described in SEQ ID NO: 7 and 8, respectively, in International Patent Publication No. WO2013096812, the contents of which are herein incorporated by reference in its entirety.

In one embodiment, the polynucleotides described herein may encode the light and/or heavy chain of pertuzumab as described in SEQ ID NO: 11 and 12, respectively, in International Patent Publication No. WO2013096812, the contents of which are herein incorporated by reference in its entirety.

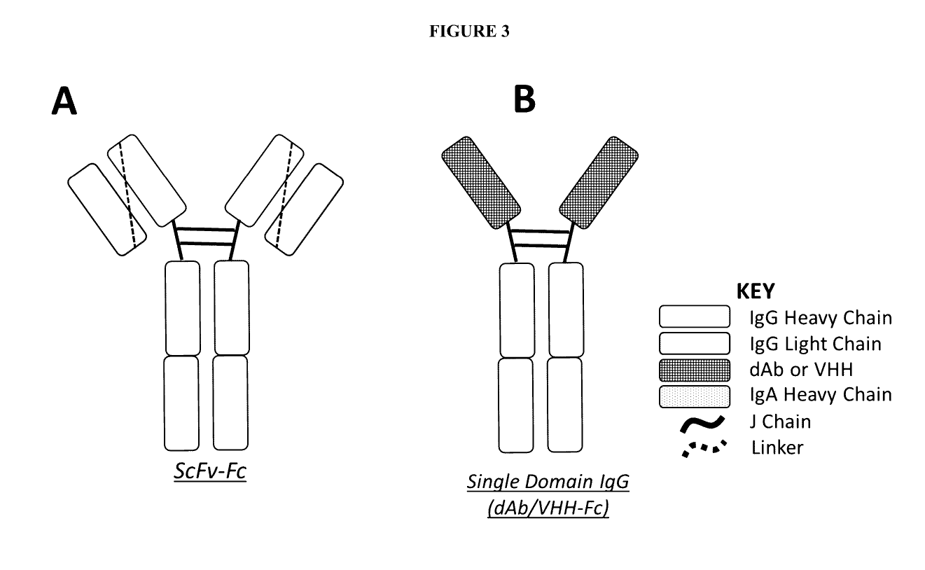

In one embodiment, the polynucleotides described herein may encode the light and/or heavy chain of Pertuzumab as described in FIGS. 3A and 3B, respectively, in International Patent Publication No. WO2013096812, the contents of which are herein incorporated by reference in its entirety.

In one embodiment, the polynucleotides described herein may encode the variant light and/or variant heavy chain of Pertuzumab as described in SEQ ID NO: 15 and 16, respectively, in International Patent Publication No. WO2013096812, the contents of which are herein incorporated by reference in its entirety.

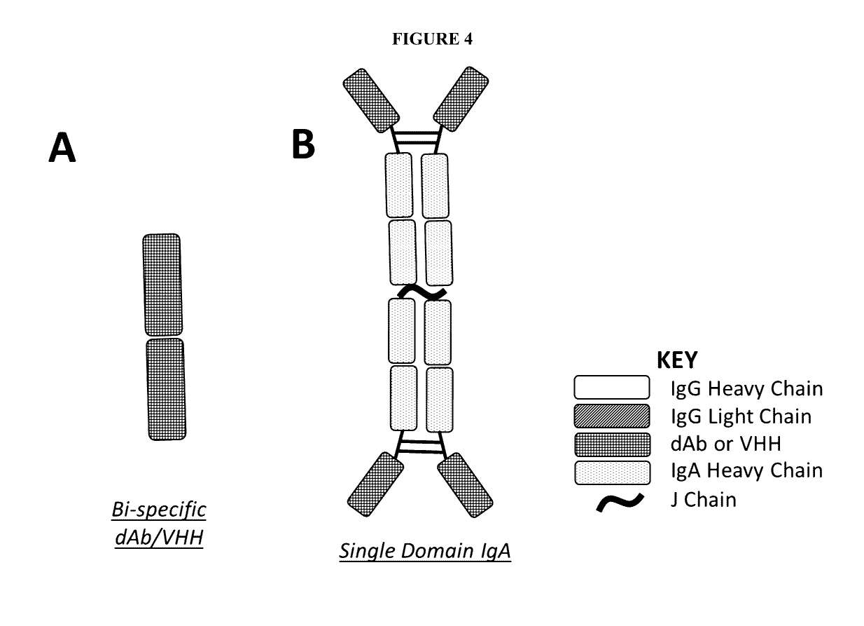

In one embodiment, the polynucleotides described herein may encode the variant light and/or variant heavy chain of pertuzumab as described in FIGS. 4A and 4B, respectively, in International Patent Publication No. WO2013096812, the contents of which are herein incorporated by reference in its entirety.

In one embodiment, the polynucleotides described herein may encode pertuzumab or a fragment or variant thereof and may not cause a side-effect, such as, but not limited to, infusion-related reactions, severe allergic reactions, diarrhea, hair loss, low levels of white blood cells with or without a fever, nausea, feeling tired, rash, damage to the nerves (numbness, tingling, pain in hands/feet), alopecia, neutropenia, and peripheral neuropathy.

In one embodiment, the polynucleotides described herein may encode pertuzumab or a fragment or variant thereof and may not cause a side-effect commonly associated with PERJETA® such as, but not limited to, infusion-related reactions, severe allergic reactions, diarrhea, hair loss, low levels of white blood cells with or without a fever, nausea, feeling tired, rash, damage to the nerves (numbness, tingling, pain in hands/feet), alopecia, neutropenia, and peripheral neuropathy.

In one embodiment, the polynucleotides described herein encoding Pertuzumab or a fragment or variant thereof are formulated for intravenous administration. As a non-limiting example, the first administration of the polynucleotides encoding pertuzumab are given over 60 minutes and subsequent treatments are given over 30-60 minutes.

In one embodiment, the polynucleotides encoding pertuzumab or a fragment or variant thereof has a half-life of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days or at least 20 days.

In one embodiment, the dose of the polynucleotides encoding pertuzumab or a fragment or variant thereof may be between 10 and 1000 mg, including, but not limited to, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg and 1000 mg. As a non-limiting example, the initial dose may be 840 mg and subsequent doses may be 420 mg.

In one embodiment, the polynucleotides encoding pertuzumab or a fragment or variant thereof are administered in combination with Herceptin (trastuxumab) and/or docetaxel (chemotherapy). As a non-limiting example, the polynucleotides encoding pertuzumab may be administered in combination with Herceptin.

In one embodiment, the polynucleotides encoding pertuzumab or a fragment or variant thereof are administered in a dosing regimen such as an initial dose followed by a subsequent dose every three weeks. As a non-limiting example, the initial dose may be 840 mg and each subsequent dose may be 420 mg.

According to the present invention, the pertuzumab polynucleotides of the present invention are engineered such that they may comprise one or more coding regions.

The coding regions of the pertuzumab polynucleotides may encode any of the regions or portions of the pertuzumab antibody. They may also further comprise coding regions not found in the original or parent pertuzumab antibody.

The pertuzumab polynucleotides of the present invention may be engineered to produce any of the 5 standard classes of immunoglobulins as shown in FIG. 1 using the pertuzumab antibody or any of its component parts as a starting molecule.

The pertuzumab polynucleotides may also be engineered according to the present invention to produce a variant pertuzumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Vedolizumab Parent Molecule or Antibody

According to the present invention, vedolizumab polynucleotides or constructs and their associated vedolizumab compositions are designed to produce the Vedolizumab antibody, a variant or a portion thereof in vivo.

Vedolizumab, also known as VDZ, MLN0002, MLN03 and LDP02, is a humanized immunoglobulin G1 monoclonal antibody that binds to the alpha4beta7 (α4β7) integrin which is a primary mediator of gastrointestinal inflammation.

Vedolizumab inhibits the binding of α4β7 to intestinal mucosal addressin cell adhesion molecule 1 (MAdCAM-1).

In one embodiment, the polynucleotides described herein encode a monoclonal antibody that binds to alpha4beta7 (α4β7) integrin to inhibit the binding of α4β7 to intestinal mucosal addressin cell adhesion molecule 1 (MAdCAM-1). As a non-limiting example, these polynucleotides may encode vedolizumab or a fragment or variant thereof. While not wishing to be bound by theory, vedolizumab may limit the ability of certain lymphocytes to infiltrate gut tissues.

In one embodiment, the polynucleotides described herein encode a monoclonal antibody that binds to alpha4beta7 (α4β7) integrin. As a non-limiting example, the polynucleotides can encode vedolizumab or a fragment or variant thereof.

In one embodiment, the polynucleotides described herein encode a modulator of the binding of α4β7 to intestinal mucosal addressin cell adhesion molecule 1 (MAdCAM-1). As a non-limiting example, the polynucleotides can encode vedolizumab or a fragment or variant thereof.

In one embodiment, the polynucleotides described herein encode a mediator of gastrointestinal inflammation. As a non-limiting example, the polynucleotides can encode vedolizumab or a fragment or variant thereof.

In one embodiment, the polynucleotides described herein encode the CAMs blocked by natalizumab. (See e.g., McLean et al. Immunotherapy. 2012 September; 4(9): 883-898. The contents of which are herein incorporated by reference in its entirety).

In one embodiment, the polynucleotides described herein may encode vedolizumab or a fragment or variant thereof. These polynucleotides may be used to treat a variety of diseases and/or disorders such as but not limited to, Crohn's disease (CD) and ulcerative colitis (UC).

In one embodiment, the polynucleotides described herein may encode vedolizumab or a fragment or variant thereof and may be used as a treatment for adults with moderately to severely active Crohn's Disease (CD).

In one embodiment, the polynucleotides described herein may encode vedolizumab or a fragment or variant thereof and may be used as a treatment of gastrointestinal inflammation.

In one embodiment, the polynucleotides described herein may encode vedolizumab or a fragment or variant thereof and may be used to limit the ability of certain lymphocytes to infiltrate gut tissues.

Certain sequences encoding vedolizumab fragments, domains or heavy or light chains are given in Table 32. The table is not an exhaustive list and any fragment or portion of the sequence may be encoded in the vedolizumab polynucleotides of the invention.

TABLE 32
Table of Vedolizumab Sequences

| | | SEQ ID |
|---|---|---|
| Description | Sequence | NO |
| Heavy Chain (See US Patent Pub | MGWSCIILFLVATATGVHSQVQLVQSG | 154 |
| US20120282249 SEQ ID NO: 2 | AEVKKPGASVKVSCKGSGYTFTSYWM | |
| and WO2008115504 and SEQ ID | HWVRQAPGQRLEWIGEIDPSESNTNYN | |
| NO: 12, the contents of each of | QKFKGRVTLTVDISASTAYMELSSLRSE | |
| which are herein incorporated by | DTAVYYCARGGYDGWDYAIDYWGQG | |
| reference in their entirety) | TLVTVSSASTKGPSVFPLAPSSKSTSGGT | |
| | AALGCLVKDYFPEPVTVSWNSGALTSG | |
| | VHTFPAVLQSSGLYSLSSVVTVPSSSLG | |
| | TQTYICNVNHKPSNTKVDKKVEPKSCD | |
| | KTHTCPPCPAPELAGAPSVFLFPPKPKD | |
| | TLMISRTPEVTCVVVDVSHEDPEVKFN | |
| | WYVDGVEVHNAKTKPREEQYNSTYRV | |
| | VSVLTVLHQDWLNGKEYKCKVSNKAL | |
| | PAPIEKTISKAKGQPREPQVYTLPPSRDE | |
| | LTKNQVSLTCLVKGFYPSDIAVEWESN | |
| | GQPENNYKTTPPVLDSDGSFFLYSKLTV | |
| | DKSRWQQGNVFSCSVMHEALHNHYTQ | |
| | KSLSLSPGK | |
| Light Chain (See US Patent Pub | MGWSCIILFLVATATGVHSDVVMTQSP | 155 |
| US20120282249 SEQ ID NO: 4 | LSLPVTPGEPASISCRSSQSLAKSYGNTY | |
| and WO2008115504 SEQ ID NO: | LSWYLQKPGQSPQLLIYGISNRFSGVPD | |
| 11, the contents of each of which | RFSGSGSGTDFTLKISRVEAEDVGVYYC | |
| are herein incorporated by | LQGTHQPYTFGQGTKVEIKRTVAAPSV | |
| reference in their entirety) | FIFPPSDEQLKSGTASVVCLLNNFYPRE | |
| | AKVQWKVDNALQSGNSQESVTEQDSK | |
| | DSTYSLSSTLTLSKADYEKHKVYACEV | |
| | THQGLSSPVTKSFNRGEC | |

In one embodiment, the signal peptide of the heavy chain is amino acids 1-19 of SEQ ID NO: 12 in International Patent Publication No WO2008115504, the contents of which are herein incorporated by reference in its entirety.

In one embodiment, the signal peptide of the light chain is amino acids 1-19 of SEQ ID NO: 11 in International Patent Publication No WO2008115504, the contents of which are herein incorporated by reference in its entirety.

In one embodiment, the polynucleotides encode one of the sequences for vedolizumab described in International Patent Publication No WO2008115504, the contents of which are herein incorporated by reference in its entirety.

In one embodiment, the polynucleotides encode one of the sequences for vedolizumab described in US Patent Publication No US20120282249, the contents of which are herein incorporated by reference in its entirety.

In one embodiment, the polynucleotides described herein may encode vedolizumab or a fragment or variant thereof and may not cause a side-effect, such as, but not limited to, upper respiratory tract infections, herpes viral infections such as oral herpes, headaches, nausea, exacerbation of ulcerative colitis, abdominal pain, fatigue and nasopharyngitis.

In one embodiment, the polynucleotides described herein may encode vedolizumab or a fragment or variant thereof and may not cause a side-effect associated with natalizumab. As a non-limiting example, the side-effect may be progressive multifocal leukoencephalopathy (PML).

In one embodiment, the polynucleotides described herein encoding vedolizumab or a fragment or variant thereof are formulated for intravenous administration.

In one embodiment, the polynucleotides encoding vedolizumab or a fragment or variant thereof has a half-life of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 day or at least 14 days.

In one embodiment, the dose of the polynucleotides encoding vedolizumab or a fragment or variant thereof may be between 0.001 and 100 mg, including, but not limited to, 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg and 100 mg.

In one embodiment, the dose of the polynucleotides encoding vedolizumab or a fragment or variant thereof may be 0.5 mg/kg, 2.0 mg/kg, 6.0 mg/kg or 10 mg/kg.

In one embodiment, the polynucleotides encoding vedolizumab or a fragment or variant thereof are administered a dose on day 1 and day 29. As a non-limiting example, the dose may be 0.5 mg/kg or 2.0 mg/kg.

In one embodiment, the polynucleotides encoding vedolizumab or a fragment or variant thereof are administered on day 1, day 15, day 43 and maintenance doses every 8 weeks thereafter. As a non-limiting example, the dose may be 2 mg/kg, 6 mg/kg or 10 mg/kg.

In one embodiment, the polynucleotides encoding vedolizumab or a fragment or variant thereof may be used as a treatment for active ulcerative colitis and patients can be administered a dose on day 1 and day 29. As a non-limiting example, the dose may be 0.5 mg/kg or 2.0 mg/kg.

In one embodiment, the polynucleotides encoding vedolizumab or a fragment or variant thereof may be used as a treatment for Crohn's Disease and patients can be administered a dose on day 1 and day 29. As a non-limiting example, the dose may be 0.5 mg/kg or 2.0 mg/kg.

According to the present invention, the vedolizumab polynucleotides of the present invention are engineered such that they may comprise one or more coding regions.

The coding regions of the vedolizumab polynucleotides may encode any of the regions or portions of the vedolizumab antibody. They may also further comprise coding regions not found in the original or parent vedolizumab antibody.

The vedolizumab polynucleotides of the present invention may be engineered to produce any of the 5 standard classes of immunoglobulins as shown in FIG. 1 using the vedolizumab antibody or any of its component parts as a starting molecule.

The vedolizumab polynucleotides may also be engineered according to the present invention to produce a variant vedolizumab antibody which is selected from one of (a) an intrabody, (b) a bicistronic or pseudobicistronic antibody, (c) a single domain antibody, either VHH or dAb, (d) a single chain variable fragment (scFv) antibody, and (e) a bispecific antibody which may comprise any two regions from two different antibodies, e.g., dAb or VHH.

### Bimagrumab Parent Molecule or Antibody

In one embodiment, the polynucleotides of the present invention may encode Bimagrumab, fragments or variants thereof.

Bimagrumab, also known as BYM338, is a novel, fully human monoclonal antibody developed as a myostatin inhibitor to treat pathological muscle loss and weakness. BYM338 was developed by the Novartis Institutes for Biomedical Research (NIBR), in collaboration with Morphosys (Martinsried, Germany). Bimagrumab binds with high affinity to type II activin receptors, preventing natural ligands such as myostatin and activin from binding. BYM338 stimulates muscle growth by blocking signaling from these inhibitory molecules.

Bimagrumab was found to bind with high affinity to type II activin receptors, preventing natural ligands (including myostatin, GDF11 and activin) from binding; thus, bimagrumab stimulates muscle growth by blocking signaling from these inhibitory molecules and inhibiting the receptor's action on muscle atrophy, thereby increasing the size and strength of a muscle.

In one embodiment, the polynucleotides described herein encode a Bimagrumab sequence or fragment thereof described in U.S. Pat. No. 8,388,968 assigned to Novartis AG (Basel, CH), the contents of which are herein incorporated by reference in its entirety.

Certain sequences encoding the polynucleotide fragments, domains or heavy or light chains for Bimagrumab are given in Table 33. The table is not an exhaustive list and any fragment or portion of the sequence which may be encoded in the polynucleotides of the invention.

TABLE 33
Table of Bimagrumab Sequences

| | | | SEQ |
|---|---|---|---|
| | | | ID |
| Description | Sequence | Source | NO. |
| Heavy chain | QVQLVQSGAEVKKPGASVKVSCKASGYTFTS | From | 156 |
| | SYINWVRQAPGQGLEWMGTINPVSGSTSYAQ | U.S. Pat. No. 8,388,968 | |
| | KFQGRVTMTRDTSISTAYMELSRLRSDDTAV | SEQ ID | |
| | YYCARGGWFDYWGQGTLVTVSSASTKGPSV | NO: 146 | |
| | FPLAPSSKSTSGGTAALGCLVKDYFPEPVTVS | | |
| | WNSGALTSGVHTFPAVLQSSGLYSLSSVVTVP | | |
| | SSSLGTQTYICNVNHKPSNTKVDKRVEPKSCD | | |
| | KTHTCPPCPAPEAAGGPSVFLFPPKPKDTLMIS | | |
| | RTPEVTCVVVDVSHEDPEVKFNWYVDGVEV | | |
| | HNAKTKPREEQYNSTYRVVSVLTVLHQDWL | | |
| | NGKEYKCKVSNKALPAPIEKTISKAKGQPREP | | |
| | QVYTLPPSREEMTKNQVSLTCLVKGFYPSDIA | | |
| | VEWESNGQPENNYKTTPPVLDSDGSFFLYSKL | | |
| | TVDKSRWQQGNVFSCSVMHEALHNHYTQKS | | |
| | LSLSPGK | | |
| Light chain | QSALTQPASVSGSPGQSITISCTGTSSDVGSYN | From | 157 |
| | YVNWYQQHPGKAPKLMIYGVSKRPSGVSNRF | U.S. Pat. No. 8,388,968 | |
| | SGSKSGNTASLTISGLQAEDEADYYCGTFAGG | SEQ ID | |
| | SYYGVFGGGTKLTVLGQPKAAPSVTLFPPSSE | NO: 141 | |
| | ELQANKATLVCLISDFYPGAVTVAWKADSSP | | |
| | VKAGVETTTPSKQSNNKYAASSYLSLTPEQW | | |
| | KSHRSYSCQVTHEGSTVEKTVAPTECS | | |

Bimagrumab is being evaluated as a potential therapeutic for Sporadic inclusion body myositis (sIBM). In one embodiment, the polynucleotides described herein may encode Bimagrumab, a fragment or variant thereof and the polynucleotides may be used in the treatment of sIBM.

Bimagrumab is being evaluated as a treatment of cachexia in stage IV metastatic non-small cell lung cancer or state III/IV adenocarcinoma of the pancreas. In one embodiment, the polynucleotides described herein may encode Bimagrumab, a fragment or variant thereof and the polynucleotides may be used in the treatment of cachexia in a subject with cancer.

Bimagrumab is being evaluated as a treatment for multiple pathological muscle loss and weakness and muscle wasting conditions such as, but not limited to, recovery from hip fracture. In one embodiment, the polynucleotides described herein may encode Bimagrumab, a fragment or variant thereof and the polynucleotides may be used in the treatment of a muscle-wasting disease of aging sarcopenia. In one embodiment, the polynucleotides described herein may encode Bimagrumab, a fragment or variant thereof and the polynucleotides may be used in the treatment of cachexia in subjects with chronic obstructive pulmonary disease (COPD). In one embodiment, the polynucleotides described herein may encode Bimagrumab, a fragment or variant thereof and the polynucleotides may be used in the treatment of muscle wasting in mechanically ventilated patients.

In some embodiments, the polynucleotides encoding bimagrumab may be used together with other antibodies specific for ActRIIB, including, but not limited to, the immunoglobulins described in U.S. Pat. Nos. 8,071,099 and 8,551,482 assigned to Novartis; as well as U.S. Pat. No. 8,268,311 assigned to Amgen; and European patent publications EP2607379, EP2468290, EP2559705, EP0771873 and the contents of each of which are incorporated by reference in their entirety.

In some embodiments, the polynucleotides encoding Bimagrumab may be used to treat ActRIIB-associated conditions, such as, but not limited to, abnormal tissue growth, developmental defects, disorders of cell growth and differentiation such as inflammation, allergy, autoimmune diseases, infectious diseases, tumors, neuromuscular disorders (e.g., muscular dystrophy and muscle atrophy), congestive obstructive pulmonary disease (and muscle wasting associated with COPD), muscle wasting syndrome, sarcopenia, cachexia, adipose tissue disorders (e.g., obesity), type 2 diabetes, bone degenerative disease (e.g., osteoporosis), musculodegenerative and neuromuscular disorders, tissue repair (e.g., wound healing), neurodegenerative diseases (e.g., amyotrophic lateral sclerosis), immunologic disorders (e.g., disorders related to abnormal proliferation or function of lymphocytes), and obesity or disorders related to abnormal proliferation of adipocytes.

In some embodiments, the compositions of the invention are used as part of a treatment for a muscular dystrophy. The term “muscular dystrophy” refers to a group of degenerative muscle diseases characterized by gradual weakening and deterioration of skeletal muscles and sometimes the heart and respiratory muscles. Muscular dystrophies are genetic disorders characterized by progressive muscle wasting and weakness that begin with microscopic changes in the muscle. As muscles degenerate over time, the person's muscle strength declines. Non-limiting examples of muscular dystrophies include: Duchenne Muscular Dystrophy (DMD), Becker Muscular Dystrophy (BMD), Emery-Dreifuss Muscular Dystrophy (EDMD), Limb-Girdle Muscular Dystrophy (LGMD), Facioscapulohumeral Muscular Dystrophy (FSH or FSHD) (also known as Landouzy-Dejerine), Myotonic Dystrophy (MMD) (also known as Steinert's Disease), Oculopharyngeal Muscular Dystrophy (OPMD), Distal Muscular Dystrophy (DD), Congenital Muscular Dystrophy (CMD).

### Evolocumab Parent Molecule or Antibody

In one embodiment, the polynucleotides of the present invention may encode Evolocumab, fragments or variants thereof.

Evolocumab, also known as AMG 145, is an investigational, fully human monoclonal antibody that inhibits proprotein convertase subtilisin/kexin type 9 (PCSK9). The PCSK9 protein targets low-density lipoprotein (LDL) receptors for degradation and thereby reduces the liver's ability to remove LDL-C, or “bad” cholesterol, from the blood.

Proprotein convertase subtilisin/kexin type 9 (PCSK9) is a serine protease produced predominantly in the liver; it is secreted into the plasma and plays a major role in regulating levels of low-density lipoprotein (LDL) cholesterol by binding to hepatic LDL receptors and promoting their degradation (Lambert, et al., J. Lipid Res., 2012, 53(12):2515-2524; Stein, et al., Curr. Atheroscler. Rep. 2013, 15:310). The PCSK9 protein binds to the liver LDL receptor and prevents the receptor from normal recycling by targeting it for degradation. By binding PCSK9, Evolocumab inhibits PCSK9 from binding to and degrading LDL receptors, and thus, LDL receptors in the liver are better able to remove LDL-C from the blood.

In one embodiment, the polynucleotides described herein encode an Evolocumab sequence or fragment thereof described in U.S. Pat. No. 8,030,457 assigned to Amgen, Inc., the contents of which are herein incorporated by reference in its entirety. As a non-limiting example, the polynucleotides described herein encoding an Evolocumab sequence may be used to treat hypercholesterolemia as described in U.S. Pat. No. 8,030,457 assigned to Amgen, Inc., the contents of which are herein incorporated by reference in its entirety.

Certain sequences encoding the polynucleotide fragments, domains or heavy or light chains for Evolocumab are given in Table 34. The table is not an exhaustive list and any fragment or portion of the sequence which may be encoded in the polynucleotides of the invention.

TABLE 34
Table of Evolocumab Sequences

| | | | SEQ |
|---|---|---|---|
| | | | ID |
| Description | Sequence | Source | NO. |
| Heavy chain | EVQLVQSGAEVKKPGASVKVSCKASGYTLTS | | 158 |
| | YGISWVRQAPGQGLEWMGWVSFYNGNTNYA | | |
| | QKLQGRGTMTTDPSTSTAYMELRSLRSDDTA | | |
| | VYYCARGYGMDVWGQGTTVTVSSASTKGPS | | |
| | VFPLAPCSRSTSESTAALGCLVKDYFPEPVTVS | | |
| | WNSGALTSGVHTFPAVLQSSGLYSLSSVVTVP | | |
| | SSNFGTQTYTCNVDHKPSNTKVDKTVERKCC | | |
| | VECPPCPAPPVAGPSVFLFPPKPKDTLMISRTP | | |
| | EVTCVVVDVSHEDPEVQFNWYVDGVEVHNA | | |
| | KTKPREEQFNSTFRVVSVLTVVHQDWLNGKE | | |
| | YKCKVSNKGLPAPIEKTISKTKGQPREPQVYT | | |
| | LPPSREEMTKNQVSLTCLVKGFYPSDIAVEWE | | |
| | SNGQPENNYKTTPPMLDSDGSFFLYSKLTVDK | | |
| | SRWQQGNVFSCSVMHEALHNHYTQKSLSLSP | | |
| | GK | | |
| Light chain | ESALTQPASVSGSPGQSITISCTGTSSDVGGYN | SEQ ID | 159 |
| | SVSWYQQHPGKAPKLMIYEVSNRPSGVSNRF | NO: 297 | |
| | SGSKSGNTASLTISGLQAEDEADYYCNSYTST | from U.S. Pat. No. | |
| | SMVFGGGTKLTVLGQPKAAPSVTLFPPSSEEL | 8,030,457 | |
| | QANKATLVCLISDFYPGAVTVAWKADSSPVK | | |
| | AGVETTTPSKQSNNKYAASSYLSLTPEQWKS | | |
| | HRSYSCQVTHEGSTVEKTVAPTECS | | |

Evolocumab is being evaluated as a potential therapeutic for hyperlipidemia. In one embodiment, the polynucleotides described herein may encode Evolocumab, a fragment or variant thereof and the polynucleotides may be used in the treatment of hyperlipidemia. In one embodiment, the polynucleotides described herein may encode Evolocumab, a fragment or variant thereof and the polynucleotides may be used in the treatment of hyperlipidemia in subjects who cannot tolerate statins.

Evolocumab is being evaluated as a potential therapeutic for subjects whose elevated cholesterol is caused by genetic disorders such as, but not limited to, heterozygous familial hypercholesterolemia and homozygous familial hypercholesterolemia. In one embodiment, the polynucleotides described herein may encode Evolocumab, a fragment or variant thereof and the polynucleotides may be used in the treatment of hyperlipidemia.

Evolocumab is being evaluated as a treatment for reducing recurrent cardiovascular events. In one embodiment, the polynucleotides described herein may encode Evolocumab, a fragment or variant thereof and the polynucleotides may be used in the treatment for reducing recurrent cardiovascular events. In one embodiment, the polynucleotides described herein may encode Evolocumab, a fragment or variant thereof and the polynucleotides may be used in the treatment for reducing recurrent cardiovascular events in subjects with hyperlipidemia at risk for cardiovascular disease.

Evolocumab is being evaluated as a treatment for coronary atherosclerosis. In one embodiment, the polynucleotides described herein may encode Evolocumab, a fragment or variant thereof and the polynucleotides may be used in the treatment of coronary atherosclerosis.

In one embodiment, the polynucleotides encoding Evolocumab may be used as an antagonist of PCSK9 polypeptide activities. In some embodiments, the polynucleotides encoding Evolocumab may be useful in treating consequences, symptoms, and/or the pathology associated with PCSK9 activity. As a non-limiting example, Evolocumab is useful in a variety of therapeutic applications, such as for treating conditions associated with PCSK9, such as cholesterol related disorders (or “serum cholesterol related disorders”) such as hypercholesterolemia.

In some embodiments, the polynucleotides encoding Evolocumab may be used to treat hypercholesterolemia, coronary heart disease, metabolic syndrome, acute coronary syndrome or related conditions.

In some embodiments, the polynucleotides encoding Evolocumab may be used together with other agents such as antibodies specific for PCSK9, or with other pharmaceutical agents, such as, but not limited to, statins.

### Alirocumab Parent Molecule or Antibody

In one embodiment, the polynucleotides of the present invention may encode Alirocumab, fragments or variants thereof.

Alirocumab, also known as REGN-727 and SAR236553, is an investigational, fully human monoclonal antibody developed as a PCSK9 inhibitor currently being developed by Sanofi and Regeneron for the treatment of hypercholesterolemia. Alirocumab is an inhibitor of Proprotein convertase subtilisin/kexin type 9 (PCSK9), a protein that targets low-density lipoprotein (LDL) receptors for degradation and thereby reduces the liver's ability to remove LDL-C, or “bad” cholesterol, from the blood. Alirocumab is in Phase III clinical trials in the United Kingdom, Europe and the United States.

Certain sequences encoding the polynucleotide fragments, domains or heavy or light chains for Alirocumab are given in Table 35. The table is not an exhaustive list and any fragment or portion of the sequence which may be encoded in the polynucleotides of the invention.

TABLE 35
Table of Alirocumab Sequences

| | | | SEQ |
|---|---|---|---|
| | | | ID |
| Description | Sequence | Source | NO. |
| Heavy chain | EVQLVESGGGLVQPGGSLRLSCAASGFTFNN | WHO Drug | 160 |
| | YAMNWVRQAPGKGLDWVSTISGSGGTTNYA | Information | |
| | DSVKGRFIISRDSSKHTLYLQMNSLRAEDTAV | publication | |
| | YYCAKDSNWGNFDLWGRGTLVTVSSASTKG | (Vol. 26, | |
| | PSVFPLAPSSKSTSGGTAALGCLVKDYFPEPVT | No. 2, 2012, | |
| | VSWNSGALTSGVHTFPAVLQSSGLYSLSSVVT | Proposed | |
| | VPSSSLGTQTYICNVNHKPSNTKVDKKVEPKS | INN: List | |
| | CDKTHTCPPCPAPELLGGPSVFLFPPKPKDTL | 107) | |
| | MISRTPEVTCVVVDVSHEDPEVKFNWYVDGV | | |
| | EVHNAKTKPREEQYNSTYRVVSVLTVLHQD | | |
| | WLNGKEYKCKVSNKALPAPIEKTISKAKGQPR | | |
| | EPQVYTLPPSRDELTKNQVSLTCLVKGFYPSDI | | |
| | AVEWESNGQPENNYKTTPPVLDSDGSFFLYSK | | |
| | LTVDKSRWQQGNVFSCSVMHEALHNHYTQK | | |
| | SLSLSPG | | |
| Light chain | DIVMTQSPDSLAVSLGERATINCKSSQSVLYR | WHO Drug | 161 |
| | SNNRNFLGWYQQKPGQPPNLLIYWASTRESG | Information | |
| | VPDRFSGSGSGTDFTLTISSLQAEDVAVYYCQ | publication | |
| | QYYTTPYTFGQGTKLEIKRTVAAPSVFIFPPSD | (Vol. 26, | |
| | EQLKSGTASVVCLLNNFYPREAKVQWKVDN | No. 2, 2012, | |
| | ALQSGNSQESVTEQDSKDSTYSLSSTLTLSKA | Proposed | |
| | DYEKHKVYACEVTHQGLSSPVTKSFNRGEC | INN: List | |
| | | 107) | |

Alirocumab is being evaluated as a potential therapeutic for reducing LDL-C levels. In one embodiment, the polynucleotides described herein may encode Alirocumab, a fragment or variant thereof and the polynucleotides may be used in the reduction of LDL-C.

Alirocumab is being evaluated as a potential therapeutic for treating high cholesterol levels in a subject. In one embodiment, the polynucleotides described herein may encode Alirocumab, a fragment or variant thereof and the polynucleotides may be used for treating high cholesterol levels in a subject.

Alirocumab is being evaluated as a potential therapeutic for treating heterozygous familial hypercholesterolemia. In one embodiment, the polynucleotides described herein may encode Alirocumab, a fragment or variant thereof and the polynucleotides may be used for treating heterozygous familial hypercholesterolemia.

Alirocumab is being evaluated as a potential therapeutic for treating acute coronary syndrome. In one embodiment, the polynucleotides described herein may encode Alirocumab, a fragment or variant thereof and the polynucleotides may be used for treating acute coronary syndrome.

In some embodiments, the polynucleotides encoding Alirocumab may be used in assays requiring specific binding to human PCSK9 or its ligands, as well as in screens to identify other antagonists of PCSK9 activity. For example, the polynucleotides encoding Alirocumab may be used in the diagnosis of PCSK9-associated diseases or conditions and screening assays to determine the presence or absence of PCSK9.

In some embodiments, the polynucleotides encoding Alirocumab may be used to treat a subject indicated for LDL apheresis, subjects with PCSK9-activating (GOF, or gain-of-function) mutations, subjects with heterozygous Familial Hypercholesterolemia (heFH), subjects with primary hypercholesterolemia who are statin intolerant or statin uncontrolled and subjects at risk for developing hypercholesterolemia.

In some embodiments, the polynucleotides encoding Alirocumab may be used to treat existing or incipient hypercholesterolemia, coronary heart disease, metabolic syndrome, acute coronary syndrome or related conditions.

In some embodiments, the polynucleotides encoding Alirlocumab may be used together with other agents such as antibodies specific for PCSK9, or with other pharmaceutical agents, such as, but not limited to, statins.

### Bococizumab Parent Molecule or Antibody

In one embodiment, the polynucleotides of the present invention may encode Bococizumab, fragments or variants thereof.

Bococizumab, also known as RN-316 and PF-04950615, is a humanized IgG2Δa monoclonal antibody developed by Pfizer Inc. Bococizumab works by blocking the function of a protein called Proprotein convertase subtilisin/kexin type 9 (PCSK9), which interferes with the clearance of low-density lipoprotein (LDL) cholesterol (LDL-C), a leading known risk factor for heart disease.

In one embodiment, the polynucleotides described herein encode at least one Bococizumab sequence described in U.S. Pat. Nos. 8,080,243; 8,399,646 and 8,426,363 by Liang et al., assigned to Rinat Neuroscience Corp. and Pfizer Inc. (the contents of each of which is herein incorporated by reference in its entirety).

Certain sequences encoding the polynucleotide fragments, domains or heavy or light chains for Bococizumab are given in Table 36. The table is not an exhaustive list and any fragment or portion of the sequence which may be encoded in the polynucleotides of the invention.

TABLE 36
Table of Bococizumab Sequences

| | | | SEQ |
|---|---|---|---|
| | | | ID |
| Description | Sequence | Source | NO. |
| Heavy chain | QVQLVQSGAEVKKPGASVKVSCKASGYTFTS | SEQ ID | 162 |
| | YYMHWVRQAPGQGLEWMGEISPFGGRTNYN | NO: 15 from | |
| | EKFKSRVTMTRDTSTSTVYMELSSLRSEDTAV | U.S. Pat. No. 8,399,646 | |
| | YYCARERPLYASDLWGQGTTVTVSSASTKGP | | |
| | SVFPLAPCSRSTSESTAALGCLVKDYFPEPVTV | | |
| | SWNSGALTSGVHTFPAVLQSSGLYSLSSVVTV | | |
| | PSSNFGTQTYTCNVDHKPSNTKVDKTVERKC | | |
| | CVECPPCPAPPVAGPSVFLFPPKPKDTLMISRT | | |
| | PEVTCVVVDVSHEDPEVQFNWYVDGVEVHN | | |
| | AKTKPREEQFNSTFRVVSVLTVVHQDWLNGK | | |
| | EYKCKVSNKGLPSSIEKTISKTKGQPREPQVYT | | |
| | LPPSREEMTKNQVSLTCLVKGFYPSDIAVEWE | | |
| | SNGQPENNYKTTPPMLDSDGSFFLYSKLTVDK | | |
| | SRWQQGNVFSCSVMHEALHNHYTQKSLSLSP | | |
| | GK | | |
| Light chain | DIQMTQSPSSLSASVGDRVTITCRASQGISSAL | SEQ ID | 163 |
| | AWYQQKPGKAPKLLIYSASYRYTGVPSRFSGS | NO: 14 from | |
| | GSGTDFTFTISSLQPEDIATYYCQQRYSLWRTF | U.S. Pat. No. 8,399,646 | |
| | GQGTKLEIKRTVAAPSVFIFPPSDEQLKSGTAS | | |
| | VVCLLNNFYPREAKVQWKVDNALQSGNSQE | | |
| | SVTEQDSKDSTYSLSSTLTLSKADYEKHKVYA | | |
| | CEVTHQGLSSPVTKSFNRGEC | | |

Bococizumab is being investigated as a lipid lowering agent for treating dyslipidemia/hyperlipidemia, hypercholesterolemia, cardiovascular disease, Heterozygous Familial Hypercholesterolemia (HeFH) and atherosclerosis. In one embodiment, the polynucleotides described herein may encode Bococizumab, a fragment or variant thereof and the polynucleotides may be used in the treatment of dyslipidemia/hyperlipidemia, hypercholesterolemia, cardiovascular disease, Heterozygous Familial Hypercholesterolemia (HeFH) or atherosclerosis.

Bococizumab is being evaluated as a potential therapeutic for treating hypercholesterolemia. In one embodiment, the polynucleotides described herein may encode Bococizumab, a fragment or variant thereof and the polynucleotides may be used for treating hypercholesterolemia.

Bococizumab is being evaluated as a potential therapeutic for lowering LDL-C. In one embodiment, the polynucleotides described herein may encode Bococizumab, a fragment or variant thereof and the polynucleotides may be used for lowering LDL-C.

Bococizumab is being evaluated as a potential therapeutic for treating heterozygous familial hypercholesterolemia. In one embodiment, the polynucleotides described herein may encode Bococizumab, a fragment or variant thereof and the polynucleotides may be used for treating heterozygous familial hypercholesterolemia.

Bococizumab is being evaluated as a potential therapeutic for treating cardiovascular disease. In one embodiment, the polynucleotides described herein may encode Bococizumab, a fragment or variant thereof and the polynucleotides may be used for treating cardiovascular disease.

Bococizumab is being evaluated as a potential therapeutic for treating atherosclerosis. In one embodiment, the polynucleotides described herein may encode Bococizumab, a fragment or variant thereof and the polynucleotides may be used for treating atherosclerosis.

Bococizumab may be used as an antagonist of PCSK9 polypeptide activities. In some embodiments, the polynucleotides encoding bococizumab may be used in treating consequences, symptoms, and/or the pathology associated with PCSK9 activity.

In some embodiment, the polynucleotides encoding bococizumab may be used to treat patients indicated for LDL apheresis, subjects with PCSK9-activating (GOF, or gain-of-function) mutations, heterozygous Familial Hypercholesterolemia (heFH), subjects with primary hypercholesterolemia who are statin intolerant or statin uncontrolled and subjects at risk for developing hypercholesterolemia.

In some embodiments, the polynucleotides encoding bococizumab may be used to treat existing or incipient hypercholesterolemia, coronary heart disease, metabolic syndrome, acute coronary syndrome or related conditions. In some embodiments, bococizumab is provided in a pharmaceutical composition or other composition comprising a pharmaceutically acceptable carrier, excipient, diluent, stabilizer, buffer, or alternative designed to facilitate administration of the antagonist in the desired amount to the treated individual.

In some embodiments, the polynucleotides encoding bococizumab may be used together with other agents such as, but not limited to, antibodies specific for PCSK9, or with other pharmaceutical agents, such as, but not limited to, statins.

### Romosozumab Parent Molecule or Antibody

In one embodiment, the polynucleotides of the present invention may encode Romosozumab, fragments or variants thereof.

Romosozumab, also known as AMG 785, AMG-785, 785A070802, CDP-7851 and CDP7851, is a humanized monoclonal antibody being developed by Amgen, Inc. and UCB Pharma S.A. (Brussels, BE) that inhibits sclerostin (the protein product of the SOST gene), increasing bone mineral density (BMD) and bone formation. Sclerostin is a bone morphogenic protein (BMP) antagonist that modulates mitogenic activity through sequestering BMPs. In sclerosteosis, a skeletal disease characterized by bone overgrowth and strong, dense bones, sclerostin is absent (Winkler et al., 2004, J. Biol. Chem. 2004, 279:36293-36298).

Sclerostin is an osteocyte-secreted glycoprotein which negatively regulates and impedes osteoblast proliferation and activity, thereby inhibiting bone formation.

In one embodiment, the polynucleotides described herein encode Sclerostin-binding antibodies described in U.S. Pat. Nos. 7,592,429; 7,872,106; 8,003,108 and 8,017,120 assigned to Amgen Inc. and UCB Pharma S.A. (Brussels, BE), the contents of each of which are herein incorporated by reference in their entirety. In one embodiment the polynucleotides described herein may be used for inhibiting bone resorption and treating osteoporosis such as by the methods of using the Sclerostin-binding antibodies described in U.S. Pat. Nos. 7,592,429; 7,872,106; 8,003,108 and 8,017,120 assigned to Amgen Inc. and UCB Pharma S.A. (Brussels, BE), the contents of each of which are herein incorporated by reference in their entirety.

Certain sequences encoding the polynucleotide fragments, domains or heavy or light chains for Romosozumab are given in Table 37. The table is not an exhaustive list and any fragment or portion of the sequence which may be encoded in the polynucleotides of the invention.

TABLE 37
Table of Romosozumab Sequences

| | | | SEQ |
|---|---|---|---|
| | | | ID |
| Description | Sequence | Source | NO. |
| Heavy chain | EVQLVQSGAEVKKPGASVKVSCKASGYTFTD | SEQ ID | 164 |
| | YNMHWVRQAPGQGLEWMGEINPNSGGAGY | NOs: 147 | |
| | NQKFKGRVTMTTDTSTSTAYMELRSLRSDDT | and 145 | |
| | AVYYCARLGYDDIYDDWYFDVWGQGTTVTV | from U.S. Pat. No. | |
| | SSASTKGPSVFPLAPCSRSTSESTAALGCLVKD | 7,592,429, | |
| | YFPEPVTVSWNSGALTSGVHTFPAVLQSSGLY | 7,872,106, | |
| | SLSSVVTVPSSNFGTQTYTCNVDHKPSNTKVD | 8,003,108 and | |
| | KTVERKCCVECPPCPAPPVAGPSVFLFPPKPK | 8,017,120 | |
| | DTLMISRTPEVTCVVVDVSHEDPEVQFNWYV | | |
| | DGVEVHNAKTKPREEQFNSTFRVVSVLTVVH | | |
| | QDWLNGKEYKCKVSNKGLPAPIEKTISKTKG | | |
| | QPREPQVYTLPPSREEMTKNQVSLTCLVKGFY | | |
| | PSDIAVEWESNGQPENNYKTTPPMLDSDGSFF | | |
| | LYSKLTVDKSRWQQGNVFSCSVMHEALHNH | | |
| | YTQKSLSLSPGK | | |
| Light chain | DIQMTQSPSSLSASVGDRVTITCRASQDISNYL | SEQ ID | 165 |
| | NWYQQKPGKAPKLLIYYTSRLLSGVPSRFSGS | NOs: 141 | |
| | GSGTDFTLTISSLQPEDFATYYCQQGDTLPYTF | and 143 | |
| | GGGTKVEIKRTVAAPSVFIFPPSDEQLKSGTAS | from U.S. Pat. No. | |
| | VVCLLNNFYPREAKVQWKVDNALQSGNSQE | 7,592,429, | |
| | SVTEQDSKDSTYSLSSTLTLSKADYEKHKVYA | 7,872,106, | |
| | CEVTHQGLSSPVTKSFNRGEC | 8,003,108 and | |
| | | 8,017,120 | |

Romosozumab is being investigated as a potential therapeutic for treating osteopenia. In one embodiment, the polynucleotides described herein may encode Romosozumab, a fragment or variant thereof and the polynucleotides may be used in the treatment of osteopenia.

Romosozumab is being investigated as a potential therapeutic for treating low bone mineral density. In one embodiment, the polynucleotides described herein may encode Romosozumab, a fragment or variant thereof and the polynucleotides may be used in the treatment of low bone mineral density.

Romosozumab is being investigated as a potential therapeutic for treating osteoporosis. In one embodiment, the polynucleotides described herein may encode Romosozumab, a fragment or variant thereof and the polynucleotides may be used in the treatment of osteoporosis.

Romosozumab is being investigated as a potential therapeutic for treating postmenopausal osteoporosis. In one embodiment, the polynucleotides described herein may encode Romosozumab, a fragment or variant thereof and the polynucleotides may be used in the treatment of postmenopausal osteoporosis.

Romosozumab is being investigated as a potential therapeutic for treating fracture healing. In one embodiment, the polynucleotides described herein may encode Romosozumab, a fragment or variant thereof and the polynucleotides may be used in the treatment of fracture healing.

In general, the polynucleotides encoding Romosozumab may be useful for treating conditions that could benefit from increased bone formation such as osteoporosis, osteopenia, genetic bone and skeletal diseases and disorders and in the treatment of bone fractures.

In some embodiments, the romosozumab antibody specifically reactive with sclerostin is useful for assays requiring specific binding to human sclerostin, as well as in screens to identify other antagonists of sclerostin activity.

Romosozumab may be used as an antagonist of PCSK9 polypeptide activities. In some embodiments, romosozumab is useful in treating consequences, symptoms, and/or the pathology associated with sclerostin activity. For example, romosozumab is useful in a variety of therapeutic applications, such as for treating conditions associated with bone loss or osteoporosis.

In some embodiments, the polynucleotides encoding Romosozumab may be used to treat conditions such as dysplasias, wherein growth or development of bone is abnormal as well as a wide variety of diseases and disorders involving osteopenia, osteoporosis and/or bone loss. Non-limiting examples of such conditions include achondroplasia, cleidocranial dysostosis, enchondromatosis, fibrous dysplasia, Gaucher's Disease, hypophosphatemic rickets, Marfan's syndrome, multiple hereditary exotoses, neurofibromatosis, osteogenesis imperfecta, osteopetrosis, osteopoikilosis, sclerotic lesions, pseudoarthrosis, and pyogenic osteomyelitis, periodontal disease, anti-epileptic drug induced bone loss, primary and secondary hyperparathyroidism, familial hyperparathyroidism syndromes, weightlessness induced bone loss, osteoporosis in men, postmenopausal bone loss, osteoarthritis, renal osteodystrophy, infiltrative disorders of bone, oral bone loss, osteonecrosis of the jaw, juvenile Paget's disease, melorheostosis, metabolic bone diseases, mastocytosis, sickle cell anemia/disease, organ transplant related bone loss, kidney transplant related bone loss, systemic lupus erythematosus, ankylosing spondylitis, epilepsy, juvenile arthritides, thalassemia, mucopolysaccharidoses, fabry disease, turner syndrome, Down Syndrome, Klinefelter Syndrome, leprosy, Perthes' Disease, adolescent idiopathic scoliosis, infantile onset multi-system inflammatory disease, Winchester Syndrome, Menkes Disease, Wilson's Disease, ischemic bone disease (such as Legg-Calve-Perthes disease, regional migratory osteoporosis), anemic states, conditions caused by steroids, glucocorticoid-induced bone loss, heparin-induced bone loss, bone marrow disorders, scurvy, malnutrition, calcium deficiency, idiopathic osteopenia or osteoporosis, congenital osteopenia or osteoporosis, alcoholism, chronic liver disease, postmenopausal state, chronic inflammatory conditions, rheumatoid arthritis, inflammatory bowel disease, ulcerative colitis, inflammatory colitis, Crohn's disease, oligomenorrhea, amenorrhea, pregnancy, diabetes mellitus, hyperthyroidism, thyroid disorders, parathyroid disorders, Cushing's disease, acromegaly, hypogonadism, immobilization or disuse, reflex sympathetic dystrophy syndrome, regional osteoporosis, osteomalacia, bone loss associated with joint replacement, HIV associated bone loss, bone loss associated with loss of growth hormone, bone loss associated with cystic fibrosis, fibrous dysplasia, chemotherapy associated bone loss, tumor induced bone loss, cancer-related bone loss, hormone ablative bone loss, multiple myeloma, drug-induced bone loss, anorexia nervosa, disease associated facial bone loss, disease associated cranial bone loss, disease associated bone loss of the jaw, disease associated bone loss of the skull, and bone loss associated with space travel. Further conditions relate to bone loss associated with aging, including facial bone loss associated with aging, cranial bone loss associated with aging, jaw bone loss associated with aging, and skull bone loss associated with aging, or related conditions.

### MABp1 Parent Molecule or Antibody

In one embodiment, the polynucleotides of the present invention may encode MABp1, fragments or variants thereof.

MABp1, also known as CV-18C3, T2-18C3 and XILONIX™, is a monoclonal antibody being developed being developed by XBiotech, Inc. that inhibits the inflammatory cytokine Interleukin 1 alpha (IL-1α), which plays a key role in interleukin-mediated tumor cell activity such as metastasis and tumor cell invasion. MABp1 may induce antibody-dependent cellular cytotoxicity (ADCC) or complement-mediated killing (CMK).

In one embodiment, the polynucleotides described herein encode Anti-IL-1α antibodies described in U.S. Pat. Nos. 8,187,817; 8,034,337; 8,242,074; 8,388,956; 8,388,969 and 8,546,331; and US Patent Application Nos. 20130177982, 20130171728, 20130078258, 20130039921, 20120276110, 20120251548, 20120231012, 20120164665, 20120114598, 20120045444, 20120021512, 20120014971, 20120015384, 20120015433, 20110311547, 20110008282, 20100068212, 20100040574, 20090298096, 20090191149 and 20090123415 assigned to XBiotech, Inc; the contents of each of which is incorporated herein by reference in its entirety. In one embodiment the polynucleotides described herein may be used for anti-IL-1 α treatment such as by the methods of using the anti-IL-1 α antibodies described in U.S. Pat. Nos. 8,187,817; 8,034,337; 8,242,074; 8,388,956; 8,388,969 and 8,546,331; and US Patent Application Nos. 20130177982, 20130171728, 20130078258, 20130039921, 20120276110, 20120251548, 20120231012, 20120164665, 20120114598, 20120045444, 20120021512, 20120014971, 20120015384, 20120015433, 20110311547, 20110008282, 20100068212, 20100040574, 20090298096, 20090191149 and 20090123415 assigned to XBiotech, Inc; the contents of each of which is incorporated herein by reference in its entirety.

Certain sequences encoding the polynucleotide fragments, domains or heavy or light chains for MABp1 are given in Table 38. The table is not an exhaustive list and any fragment or portion of the sequence which may be encoded in the polynucleotides of the invention.

TABLE 38
Table of MABp1 Sequences

| | | | SEQ |
|---|---|---|---|
| | | | ID |
| Description | Sequence | Source | NO. |
| Heavy chain | MEFGLSWVFLVALLRGVQCQVQLVESGGGV | SEQ ID NO: | 166 |
| | VQPGRSLRLSCTASGFTFSMFGVHWVRQAPG | 9 from U.S. Pat. No. | |
| | KGLEWVAAVSYDGSNKYYAESVKGRFTISRD | 8,034,337, | |
| | NSKNILFLQMDSLRLEDTAVYYCARGRPKVVI | 8,388,956 and | |
| | PAPLAHWGQGTLVTFSSASTKGPSVFPLAPSS | 8,388,969 | |
| | KSTSGGTAALGCLVKDYFPEPVTVSWNSGAL | and | |
| | TSGVHTFPAVLQSSGLYSLSSVVTVPSSSLGTQ | SEQ ID NO: | |
| | TYICNVNHKPSNTKVDKRVEPKSCDKTHTCPP | 3 from | |
| | CPAPELLGGPSVFLFPPKPKDTLMISRTPEVTC | 8,242,074 and | |
| | VVVDVSHEDPEVKFNWYVDGVEVHNAKTKP | 8,546,331 | |
| | REEQYNSTYRVVSVLTVLHQDWLNGKEYKC | | |
| | KVSNKALPAPIEKTISKAKGQPREPQVYTLPPS | | |
| | REEMTKNQVSLTCLVKGFYPSDIAVEWESNG | | |
| | QPENNYKTTPPVLDSDGSFFLYSKLTVDKSRW | | |
| | QQGNVFSCSVMHEALHNHYTQKSLSLSPGK | | |
| Light chain | MDMRVPAQLLGLLLLWFPGSRCDIQMTQSPS | SEQ ID NO: | 167 |
| | SVSASVGDRVTITCRASQGISSWLAWYQQKP | 11 from U.S. Pat. No. | |
| | GKAPKLLIYEASNLETGVPSRFSGSGSGSDFTL | 8,034,337 | |
| | TISSLQPEDFATYYCQQTSSFLLSFGGGTKVEH | | |
| | KRTVAAPSVFIFPPSDEQLKSGTASVVCLLNNF | | |
| | YPREAKVQWKVDNALQSGNSQESVTEQDSK | | |
| | DSTYSLSSTLTLSKADYEKHKVYACEVTHQG | | |
| | LSSPVTKSFNRGEC | | |

MABp1 is being investigated as a potential therapeutic for blocking interleukin-1 alpha activity and/or interrupting the inflammatory response that supports tumor growth/metastasis in subject with late stage cancer. In one embodiment, the polynucleotides described herein may encode MABp1, a fragment or variant thereof and the polynucleotides may be used for blocking interleukin-1 alpha activity and/or interrupting the inflammatory response that supports tumor growth/metastasis in subject with late stage cancer.

MABp1 is being investigated as a potential therapeutic for treating diabetes. In one embodiment, the polynucleotides described herein may encode MABp1, a fragment or variant thereof and the polynucleotides may be used in the treatment of diabetes.

MABp1 is being investigated as a potential therapeutic for treating skin conditions such as, but not limited to, acne, psoriasis and Pyoderma Gangrenosum (PG). In one embodiment, the polynucleotides described herein may encode MABp1, a fragment or variant thereof and the polynucleotides may be used in the treatment of skin conditions such as, but not limited to, acne, psoriasis and Pyoderma Gangrenosum (PG).

MABp1 is being investigated as a potential therapeutic for treating Pyoderma Gangrenosum (PG). In one embodiment, the polynucleotides described herein may encode MABp1, a fragment or variant thereof and the polynucleotides may be used in the treatment of PG. PG is an inflammatory skin disorder that causes tissue necrosis and results in severe, painful ulcers, most commonly on the legs. PG is considered a rare condition, affecting approximately 1 in 100,000 persons, but can be devastating to those afflicted. In approximately 50% of cases, PG occurs secondary to an underlying disease such as inflammatory bowel disease, systemic arthritis, hematological diseases and malignancies.

In some embodiments, the polynucleotides encoding MABp1 may be used in the treatment of consequences, symptoms, and/or the pathology associated with IL-1α activity. As a non-limiting example, MABp1 may be used for treating or ameliorating diseases and disorders involving inflammation or infection, or conditions associated with cancer, such as cachexia, including (but not limited to): cutaneous inflammation, inflammatory skin diseases with epidermal injury, psoriasis, palmoplantar pustulosis, pustular psoriasis, acne vulgaris, pemphigus and lichen planus; contact and delayed-type hypersensitivity reactions, and autoimmune diseases, such as experimental autoimmune encephalomyelitis, multiple sclerosis, diabetes, vascular disorders, such as vasculitis; coronary arteriosclerosis, juvenile idiopathic arthritis, rheumatoid arthritis, cancer and tumor metastasis, fever, periodontitis, Alzheimer Disease and Pyoderma gangrenosum (PG).

In some embodiments, the polynucleotides encoding MABp1 may be used to treat conditions such as dysplasias, wherein growth or development of tissues is abnormal as well as a wide variety of diseases and disorders involving pseudoarthrosis, and pyogenic osteomyelitis, periodontal disease, mastocytosis, sickle cell anemia/disease, organ transplant and rejection, systemic lupus erythematosus, ankylosing spondylitis, epilepsy, juvenile arthritides, thalassemia, mucopolysaccharidoses, fabry disease, turner syndrome, Down Syndrome, Klinefelter Syndrome, leprosy, Perthes' Disease, adolescent idiopathic scoliosis, infantile onset multi-system inflammatory disease, Winchester Syndrome, Menkes Disease, Wilson's Disease, ischemic disease, anemic states, conditions caused by steroids, alcoholism, chronic liver disease, postmenopausal state, chronic inflammatory conditions, rheumatoid arthritis, inflammatory bowel disease, ulcerative colitis, inflammatory colitis, Crohn's disease, oligomenorrhea, amenorrhea, pregnancy, diabetes mellitus, hyperthyroidism, thyroid disorders, parathyroid disorders, Cushing's disease, acromegaly, hypogonadism, immobilization or disuse, reflex sympathetic dystrophy syndrome, fibrous dysplasia, chemotherapy associated inflammation, multiple myeloma, cachexia and anorexia nervosa, or related conditions.

### Gevokizumab Parent Molecule or Antibody

In one embodiment, the polynucleotides of the present invention may encode Gevokizumab, fragments or variants thereof.

Gevokizumab, also known as D09911, XOMA 052/S 78989, XOMA-052, XMA 005.2 and UNII-QX3JU54GYQ, is a humanized monoclonal antibody that targets interleukin 1β designed by XOMA Corporation, and being developed and commercialized under a joint agreement between XOMA Corporation and Servier (an independent pharmaceutical research company in Suresnes, France) to treat inflammation and autoinflammatory disorders. Gevokizumab is an anti-inflammatory drug candidate that targets the pro-inflammatory cytokine interleukin-1 beta, a primary trigger of pathologic inflammation in multiple diseases.

IL-1 signaling may play a role in diabetes and obesity. Inflammation plays a causal role in insulin resistance, and in rodent models targeting inflammatory cytokine production through genetic and pharmacological approaches results in improvements in insulin signaling. After insulin binds to the insulin receptor, insulin initiates signaling cascades that activate downstream pathways, notably PI3K-AKT and the mitogenic MAP kinase-ERK pathways. The reduction of IL-1 signaling was found to improve adipose tissue insulin sensitivity, implicating inflammasome activation and IL-1 signaling in obesity (Grant, et al., 2013, Front. Immunol. 4(50): 1-10; the contents of which are herein incorporated by reference in its entirety).

In one embodiment, the polynucleotides described herein encode Interleukin 1β-binding antibodies described in WO2007002261 and U.S. Pat. Nos. 7,531,166; 7,582,742; 7,695,717; 7,744,865; 7,744,866; 7,829,093; 7,829,094; 7,943,121; 7,988,968 and 8,377,442 assigned to XOMA Technology, Ltd; the contents of each of which is incorporated herein by reference in its entirety. In one embodiment the polynucleotides described herein may be used for treatment of Interleukin 1β related disease or condition such as by the methods of using the Interleukin 1β-binding antibodies described in WO2007002261 and U.S. Pat. Nos. 7,531,166; 7,582,742; 7,695,717; 7,744,865; 7,744,866; 7,829,093; 7,829,094; 7,943,121; 7,988,968 and 8,377,442 assigned to XOMA Technology, Ltd; the contents of each of which is incorporated herein by reference in its entirety.

Certain sequences encoding the polynucleotide fragments, domains or heavy or light chains for Gevokizumab are given in Table 39. The table is not an exhaustive list and any fragment or portion of the sequence which may be encoded in the polynucleotides of the invention.

TABLE 39
Table of Gevokizumab Sequences

| | | | SEQ |
|---|---|---|---|
| | | | ID |
| Description | Sequence | Source | NO. |
| Heavy chain | QVQLQESGPGLVKPSQTLSLTCSFSGFSLSTSG | SEQ ID NO: | 168 |
| | MGVGWIRQPSGKGLEWLAHIWWDGDESYNP | 15 from PCT | |
| | SLKSRLTISKDTSKNQVSLKITSVTAADTAVYF | Publication | |
| | CARNRYDPPWFVDWGQGTLVTVSSASTKGPS | WO | |
| | VFPLAPCSRSTSESTAALGCLVKDYFPEPVTVS | 2007/002261 | |
| | WNSGALTSGVHTFPAVLQSSGLYSLSSVVTVT | | |
| | SSNFGTQTYTCNVDHKPSNTKVDKTVERKCC | | |
| | VECPPCPAPPVAGPSVFLFPPKPKDTLMISRTP | | |
| | EVTCVVVDVSHEDPEVQFNWYVDGMEVHNA | | |
| | KTKPREEQFNSTFRVVSVLTVVHQDWLNGKE | | |
| | YKCKVSNKGLPAPIEKTISKTKGQPREPQVYT | | |
| | LPPSREEMTKNQVSLTCLVKGFYPSDIAVEWE | | |
| | SNGQPENNYKTTPPMLDSDGSFFLYSKLTVD | | |
| | KSRWQQGNVFSCSVMHEALHNHYTQKSLSLS | | |
| | PG | | |
| Light chain | DIQMTQSTSSLSASVGDRVTITCRASQDISNYL | SEQ ID NO: | 169 |
| | SWYQQKPGKAVKLLIYYTSKLHSGVPSRFSGS | 11 from PCT | |
| | GSGTDYTLTISSLQQEDFATYFCLQGKMLPWT | Publication | |
| | FGQGTKLEIKRTVAAPSVFIFPPSDEQLKSGTA | WO | |
| | SVVCLLNNFYPREAKVQWKVDNALQSGNSQ | 2007/002261 | |
| | ESVTEQDSKDSTYSLSSTLTLSKADYEKHKVY | | |
| | ACEVTHQGLSSPVTKSFNRGEC | | |

In one embodiment, the polynucleotides described herein may encode Gevokizumab, a fragment or variant thereof and the polynucleotides may be used for the treatment of moderate to severe inflammatory acne vulgaris, erosive inflammatory osteoarthritis of the hand, and/or non-anterior scleritis.

Erosive osteoarthritis of the hand (EOA) is caused by the breakdown of the body's natural balance between cartilage formation and degradation, which leads to the narrowing of the space between the first and second joints in the fingers. Patients with EOA experience high degrees of pain, including throbbing, swelling, and prolonged periods of morning stiffness. Over time, the joints become deformed, impacting hand function and ultimately reducing EOA patients' quality of life. Approximately two million people in the U.S. have been diagnosed with EOA, and the disease affects women twelve times more often than men for reasons that are not understood by the scientific or medical community. Two Phase 2 proof-of-concept studies were conducted to determine whether gevokizumab was effective in the treatment of inflammatory EOA of the hand (ClinicalTrials.gov Identifiers: NCT01882491 and NCT01683396).

In one embodiment, the polynucleotides described herein may encode Gevokizumab, a fragment or variant thereof and the polynucleotides may be used for the treatment of pyoderma gangrenosum, pustular psoriasis, moderate to severe inflammatory acne, erosive osteoarthritis of the hand, active non-infectious scleritis or autoimmune inner ear disease.

Pyoderma gangrenosum (PG) is a rare neutrophilic dermatosis of painful expanding necrotic skin ulcers, which has four classifications based upon the type of skin ulcers manifested. The U.S. Department of Health and Human Services' National Institutes of Health's Office of Rare Disease Research lists PG occurring in about 1 per 100,000 people. Approximately 50 to 70 percent of the PG patient population has an underlying systemic condition, while the remainder is idiopathic (unknown cause). The most prevalent underlying condition is inflammatory bowel disease (IBD), most commonly ulcerative colitis and Crohn's disease. The prognosis for PG is directly linked to the patient's response to therapy for the underlying disease. Patients receive a combination of topical and systemic therapy to treat the ulcers, which may take up to two years to heal. Despite the ongoing use of systemic therapy, up to 46 percent of patients experience a relapse.

In one embodiment, the polynucleotides described herein may encode Gevokizumab, a fragment or variant thereof and the polynucleotides may be used to reduce arterial wall inflammation in a subject with atherosclerotic plaque inflammation.

In one embodiment, the polynucleotides described herein may encode Gevokizumab, a fragment or variant thereof and the polynucleotides may be used for the treatment of polymyositis/dermatomyositis.

In one embodiment, the polynucleotides described herein may encode Gevokizumab, a fragment or variant thereof and the polynucleotides may be used for the treatment of Behcet's disease. Behcet's disease causes chronic inflammation of the blood vessels, or vasculitis, among other complications. Uveitis is a vasculitis of the blood vessels in the eye which can be vision-threatening. Behcet's uveitis is one of the most severe forms of uveitis which can lead to blindness and affects approximately 60% of Behcet's disease patients. There are at least 250,000 patients diagnosed with Behcet's disease worldwide. Onset of the disease occurs most commonly in adults in their twenties, thirties and forties, and is typically more severe in men. Without immediate treatment, major exacerbations of Behcet's uveitis may lead to retinal detachment, macular edema, vitreous hemorrhage, glaucoma and eventual blindness. The effects of these exacerbations on vision are cumulative. Patients often experience multiple exacerbations per year, requiring treatment to control the frequency and severity of attacks of this chronic disease. As a non-limiting example, the polynucleotides described herein encoding Gevokizumab may be used to prevent disease flares in subjects with Behcet's disease.

In one embodiment, the polynucleotides described herein may encode Gevokizumab, a fragment or variant thereof and the polynucleotides may be used for the treatment of diabetes.

In one embodiment, the polynucleotides described herein may encode Gevokizumab, a fragment or variant thereof and the polynucleotides may be used for the treatment of Familial Cold Autoinflammatory Syndrome (FCAS)/Muckle-Wells Syndrome (MWS).

In one embodiment, the polynucleotides described herein may encode Gevokizumab, a fragment or variant thereof and the polynucleotides may be used for the treatment of scleritis. Scleritis is a chronic, painful and potentially blinding inflammatory disease characterized by edema of the episcleral and sclera tissues (the white outer coating of the eye) and is commonly associated with systemic autoimmune disorders. In severe cases, it can cause blindness. It is commonly associated with autoimmune disorders such as rheumatoid arthritis. Mild scleritis can be treated with drugs such as ibuprofen. More severe scleritis may need oral steroids or immunosuppressive treatments; however, these treatments can cause side effects in the whole body

In one embodiment, the polynucleotides described herein encoding Gevokizumab may be used to treat or prevent any IL-I related disease or condition. As a non-liming example, the disease or condition may be inflammatory conditions, allergies and allergic conditions, cancers, hypersensitivity reactions, autoimmune diseases, severe infections, and organ or tissue transplant rejection. IL-I related conditions include rheumatoid arthritis (RA), osteoarthritis, Crohn's disease, ulcerative colitis (UC), septic shock, chronic obstructive pulmonary disease (COPD), asthma, graft versus host disease, atherosclerosis, adult T cell leukemia, multiple myeloma, multiple sclerosis, stroke, Alzheimer's disease, neonatal Onset Multisystem Inflammatory Disorder (NOMID/CINCA), systemic onset juvenile idiopathic arthritis, Stills disease, CAPS, Muckle-Wells syndrome, prevent rheumatoid arthritis, osteoarthritis, Crohn's disease, ulcerative colitis, septic shock, chronic obstructive pulmonary disease, asthma, graft versus host disease, atherosclerosis, adult T cell leukemia, multiple myeloma, multiple sclerosis, stroke, Alzheimer's disease, systemic onset juvenile idiopathic arthritis, rheumatoid arthritis, osteoarthritis, atherosclerosis, myasthenia gravis, acute pancreatitis; ALS; cachexia/anorexia, including AIDS-induced cachexia; asthma and other pulmonary diseases; autoimmune vasculitis; CIAS1 Associated Periodic Syndromes (CAPS); chronic fatigue syndrome; Clostridium associated illnesses, including Clostridium-associated diarrhea; coronary conditions and indications, including congestive heart failure, coronary restenosis, myocardial infarction, myocardial dysfunction (e.g., related to sepsis), and coronary artery bypass graft; cancers, such as multiple myeloma and myelogenous (e.g., AML and CIVIL) and other leukemias, as well as tumor metastasis; diabetes (e.g., insulin diabetes); endometriosis; familial Cold Autoinflammatory Syndrome (FCAS); familial mediterranean fever (FMF); fever; fibromyalgia; glomerulonephritis; graft versus host disease/transplant rejection; hemohorragic shock; hyperalgesia; inflammatory bowel disease; inflammatory conditions of a joint, including psoriatic arthritis (as well as osteoarthritis and rheumatoid arthritis); inflammatory eye disease, as may be associated with, for example, corneal transplant; ischemia, including cerebral ischemia (e.g., brain injury as a result of trauma, epilepsy, hemorrhage or stroke, each of which may lead to neurodegeneration); Kawasaki's disease; learning impairment; lung diseases (e.g., ARDS); myopathies (e.g., muscle protein metabolism, especially in sepsis); neurotoxicity (e.g., as induced by HIV); osteoporosis; pain, including cancer-related pain; Parkinson's disease; periodontal disease; pre-term labor; psoriasis; reperfusion injury; side effects from radiation therapy; sleep disturbance; temporal mandibular joint disease; tumor necrosis factor receptor-associated periodic fever syndrome (TRAPS); uveitis; or an inflammatory condition resulting from strain, sprain, cartilage damage, trauma, orthopedic surgery, infection or other disease processes.

### Mepolizumab Parent Molecule or Antibody

In one embodiment, the polynucleotides of the present invention may encode Mepolizumab, fragments or variants thereof.

Mepolizumab, also known as D09 Bosatria, SB-240563, L04AC06, is a fully humanized IgG1 kappa which targets IL-5 under development by GlaxoSmithKline. Mepolizumab blocks IL-5 binding the IL-5 receptor (IL-5R), thereby preventing the stimulation of growth of eosinophils present in the lung in eosinophilic asthma. Mepolizumab is currently in under investigation in clinical trials for asthma, and chronic obstructive pulmonary disease (COPD) with eosinophilic bronchitis, and eosinophilic granulomatosis with polyangiitis (EGPA).

IL-5 is hailed as a promising target to prevent/decrease eosinophil-mediated inflammation in patients with asthma and other eosinophil-related conditions (see e.g., International Patent Publications WO2008134724 and WO2009120927, the contents of each of which are incorporated herein by reference in its entirety).

Mepolizumab blocks binding of human IL-5 to the alpha subunit of the IL-5 receptor complex expressed on the cell surface of eosinophils. In preclinical studies of Mepolizumab in monkeys, in vivo pharmacologic activity leading to decreases in eosinophil counts was observed (Hart et al, 1990). While not wishing to be bound by theory, Mepolizumab may function by reducing eosinophil accumulation, and reducing/reversing airway remodeling in eosinophilic asthma.

In one embodiment, the polynucleotides described herein encode any of the IL-5 antibody sequences, fragments or variants thereof described in International Patent Publication No. WO2009120927 and WO2009068649, the contents of which are herein incorporated by reference in its entirety. As a non-limiting example, the polynucleotides described herein may encode the heavy chain sequence described as SEQ ID NO: 19 in International Patent Publication No. WO2009120927, the contents of which are herein incorporated by reference in its entirety. As a non-limiting example, the polynucleotides described herein may encode the light chain sequence described as SEQ ID NO: 21 in International Patent Publication No. WO2009120927, the contents of which are herein incorporated by reference in its entirety. As a non-limiting example, the polynucleotides described herein may encode the heavy chain sequence described as SEQ ID NO: 65 or 191 in International Patent Publication No. WO2009068649, the contents of which are herein incorporated by reference in its entirety. As a non-limiting example, the polynucleotides described herein may encode the light chain sequence described as SEQ ID NO: 66 in International Patent Publication No. WO2009068649, the contents of which are herein incorporated by reference in its entirety.

Certain sequences encoding the polynucleotide fragments, domains or heavy or light chains for Mepolizumab are given in Table 40. The table is not an exhaustive list and any fragment or portion of the sequence which may be encoded in the polynucleotides of the invention.

TABLE 40
Table of Mepolizumab Sequences

| | | | SEQ |
|---|---|---|---|
| | | | ID |
| Description | Sequence | Source | NO. |
| Heavy chain | QVTLRESGPALVKPTQTLTLTCTVSGFSL | From | 170 |
| sequence | TSYSVHWVRQPPGKGLEWLGVIWASGG | WO2009120927 | |
| | TDYNSALMSRLSISKDTSRNQVVLTMTN | SEQ ID NO: 19 | |
| | MDPVDTATYYCARDPPSSLLRLDYWGR | | |
| | GTPVTVSS | | |
| Light chain | DIVMTQSPDSLAVSLGERATINCKSSQSL | From | 171 |
| sequence | LNSGNQKNYLAWYQQKPGQPPKLLIYGA | WO2009120927 | |
| | STRESGVPDRFSGSGSGTDFTLTISSLQAE | SEQ ID NO: 21 | |
| | DVAVYYCQNVHSFPFTFGGGTKLEIK | | |
| Heavy chain | QVTLRESGPALVKPTQTLTLTCTVSGFSL | From 172 | |
| sequence | TSYSVHWVRQPPGKGLEWLGVSRIWASG | WO2009068649 | |
| | GTDYNSALMLSISKDTSRNQVVLTMTNM | SEQ ID NO: 65 | |
| | DPVDTATYYCARDPPSSLLRLDYWGRGT | | |
| | LVTVSSASTKGPSVFPLAPSSKSTSGGTA | | |
| | ALGCLVKDNSGALTYFPEPVTVSWSGVH | | |
| | TFPAVLQSSGLYSLSSVVTVPSSSLGTQT | | |
| | YICNVNHKPSNTKVDKKVEPKSCDKTHT | | |
| | CPPCPAPELLGGPSVFLFPPKPKDTLMISR | | |
| | TPEVTCVVVDVSHEDPEVKFNWYVDGV | | |
| | EVHNAKTKPREEQYNSTYRVVSVLTVLH | | |
| | QDWLNGKEYKCKVSNKALPAPIEKTISK | | |
| | AKGQLPPSRDELPREPQVYTTKNQVSLTC | | |
| | LVKGFYPSDIAVEWESNGQPENNYKTTPP | | |
| | VLDSDGSFFLYSKLTVDKSRWQQGNVFS | | |
| | CSVMHEALHNHYTQKSLSLSPGK | | |
| Heavy chain | QVTLRESGPALVKPTQTLTLTCTVSGFSL | From | 173 |
| sequence | TSYSVHWVRQPPGKGLEWLGVIWASGG | WO2009068649 | |
| | TDYNSALMSRLSISKDTSRNQVVLTMTN | SEQ ID NO: 191 | |
| | MDPVDTATYYCARDPPSSLLRLDYWGR | | |
| | GTPVTVSSASTKGPSVFPLAPSSKSTSGGT | | |
| | AALGCLVKDNSGALTYFPEPVTVSWSGV | | |
| | HTFPAVLQSSGLYSLSSVVTVPSSSLGTQ | | |
| | TYICNVNHKPSNTKVDKRVEPKSCDKTH | | |
| | TCPPCPAPELLGGPSVFLFPPKPKDTLMIS | | |
| | RTPEVTCVVVDVSHEDPEVKFNWYVDG | | |
| | VEVHNAKTKPREEQYNSTYRVVSVLTVL | | |
| | HQDWLNGKEYKCKVSNKALPAPIEKTIS | | |
| | KAKGQPREPQVYTLPPSREEMTKNQVSL | | |
| | TCLVKGFYPSDIAVEWESNGQPDSDGSE | | |
| | NNYKTTPPVLFFLYSKLTVDKSRWQQGN | | |
| | VFSCSVMHEALHNHYTQKSLSLSPGK | | |
| Light chain | DIVMTQSPDSLAVSLGERATINCKSSQSL | From | 174 |
| sequences | LNSGNQKNYLAWYQQKPGQPPKLLIYGA | WO2009068649 | |
| | STRESGVPDRFSGSGSGTDFTLTISSLQAE | SEQ ID NO: 66 | |
| | DVAVYYCQNVHSFPFTFGGGTKLEIKRT | | |
| | VAAPSVFIFPPSDEQLKSGTASVVCLLNN | | |
| | FYPREAKVQWKVDNALQSGNSQESVTE | | |
| | QDSKDSTYSLSSTLTLSKEKHKVYACEVT | | |
| | HQSPVTKSFNRGEC | | |
| Heavy chain | SYSVH | From | 175 |
| CDR1 | | WO2009120927 | |
| | | SEQ ID NO: 7 | |
| Heavy chain | VIWASGGTDYNSALMS | From | 176 |
| CDR2 | | WO2009120927 | |
| | | SEQ ID NO: 8 | |
| Heavy chain | DPPSSLLRLDY | From | 177 |
| CDR3 | | WO2009120927 | |
| | | SEQ ID NO: 9 | |
| Light chain | KSSQSLLNSGNQKNYLA | From | 178 |
| CDR1 | | WO2009120927 | |
| | | SEQ ID NO: 10 | |
| Light chain | GASTRES | From | 179 |
| CDR2 | | WO2009120927 | |
| | | SEQ ID NO: 11 | |
| Light chain | QNVHSFPFT | From | 180 |
| CDR3 | | WO2009120927 | |
| | | SEQ ID NO: 12 | |

In one embodiment, the polynucleotides described herein may encode Mepolizumab, a fragment or variant thereof and the polynucleotides may be used for the treatment of asthma.

In one embodiment, the polynucleotides described herein may encode Mepolizumab, a fragment or variant thereof and the polynucleotides may be used to mediate eosinophil proliferation, differentiation, maturation, migration to tissues sites and/or prevention of apoptosis. As a non-limiting example, the polynucleotides described herein may encode Mepolizumab may be used to treat, prevent and/or decrease eosinophil-mediated inflammation in patients with asthma and other eosinophil-related conditions such as by the methods described in International Patent Publication Nos. WO2008134724, WO2009120927, the contents of each of which are incorporated herein by reference in its entirety.

In one embodiment, polynucleotides encoding Mepolizumab, fragments or variants thereof may be used to prevent or treat a disease with excess eosinophil production selected from the group consisting of atopic asthma, atopic dermatitis, allergic rhinitis, non-allergic rhinitis, asthma, severe asthma, chronic eosinophilic pneumonia, allergic bronchopulmonary aspergillosis, coeliac disease, Churg-Strauss syndrome, eosinophilic myalgia syndrome, hypereosinophilic syndrome, oedematous reactions including episodic angiodema, helminth infections, onchocercal dermatitis eosinophilic oesophagitis, eosinophilic gastritis, eosinophilic gastroenteritis, eosinophilic enteritis, eosinophilic colitis, nasal micropolyposis, nasal polyposis, aspirin intolerance asthma, obstructive sleep apnoe, chronic asthma, Crohn's disease, scleroderma and endomyocardial fibrosis.

In one embodiment, the polynucleotides described herein may encode Mepolizumab, a fragment or variant thereof and the polynucleotides may be used to reduce blood and sputum eosinophil numbers.

In one embodiment, the polynucleotides described herein may encode Mepolizumab, a fragment or variant thereof and the polynucleotides may be used for the treatment of eosinophilic bronchitis. As a non-limiting example, the polynucleotides described herein may encode Mepolizumab, a fragment or variant thereof and the polynucleotides may be used for the treatment of eosinophilic bronchitis in a subject with chronic obstructive pulmonary diseases (COPD).

In one embodiment, the polynucleotides described herein may encode Mepolizumab, a fragment or variant thereof and the polynucleotides may be used for the treatment of FIP1L1/PDGFRA-negative hypereosinophilic syndromes, eosinophilic esophagitis, nasal polyposis and Churg-Strauss syndrome (Wechsler et al, 2012, the content of which is herein incorporated in its entirety).

In one embodiment, the polynucleotides described herein may encode Mepolizumab, a fragment or variant thereof and the polynucleotides may be used for the treatment of nasal polyposis. As a non-limiting example, nasal polyposis may be severe nasal polyposis.

In one embodiment, the polynucleotides described herein may encode Mepolizumab, a fragment or variant thereof and the polynucleotides may be used for the treatment of atopic dermatitis.

In one embodiment, the polynucleotides described herein may encode Mepolizumab, a fragment or variant thereof and the polynucleotides may be used for the treatment of Hypereosinophilic syndromes (HES). Hypereosinophilic syndromes (HES) are a rare group of disorders characterized by persistent blood eosinophilia (>1500/mm³) and evidence of eosinophil-related end-organ pathology. HES include, but are not limited to, idiopathic HESs, Churg-Strauss syndrome (CSS)-related vasculitis, and eosinophil-associated gastrointestinal disorders (EGID). Standard treatment includes corticosteroids and is effective, at least initially, in most patients.

In one embodiment, polynucleotides encoding Mepolizumab, fragments or variants thereof may be used to prevent or treat diseases with airway eosinophilia, eosinophilic asthma, eosinophilic COPD, eosinophilic bronchitis, and airway eosinophilia associated with viral infections, such as, but not limited to, rhino virus.

In one embodiment, polynucleotides encoding Mepolizumab, fragments or variants thereof may be used in combination with a second an anti-asthma medication (corticosteroids, non-steroidal agents, beta agonists, leukotriene antagonists, xanthines, fluticasone, salmeterol, albuterol) which may be delivered by inhalation or other appropriate means.

In one embodiment, polynucleotides encoding Mepolizumab, fragments or variants thereof may be administered in combination with a further IL-5 antagonist, including but not limited to mepolizumab and reslizumab or benralizumab or antibodies described in International Patent Publication Nos. WO2008134724 and WO2009120927, the contents of each of which are incorporated herein by reference in its entirety, alone or in combination with a corticosteroid.

### Reslizumab Parent Molecule or Antibody

In one embodiment, the polynucleotides of the present invention may encode Reslizumab, fragments or variants thereof.

Reslizumab, also known as CINQUIL™, SCH 55700, CDP-835 is a humanized monoclonal anti-interleukin 5 (IL-5) antibody currently under development by Teva for pediatric and adult eosinophilic asthma. IL-5 is an important mediator of eosinophil maturation, proliferation, differentiation, and survival. Reslizumab blocks IL-5 binding the IL-5 receptor (IL-5R), thereby preventing the stimulation of growth of eosinophils present in the lung in eosinophilic asthma.

Reslizumab is a humanized form of the rat antibody 39D10, a rat mAb with a K_{d} of 53 pM against human IL-5. To develop Reslizumab, 39D10 was humanized using complementarity determining region grafting technology into a human framework with a κ light chain and a γ4 constant region. The humanized antibody, designated Sch 55700, was shown to retain the potency of the parent antibody by blocking IL-5 receptor (IL-5R) binding and by inhibiting IL-5-induced cell proliferation (Zhang et al, 1999 and International Patent Publication WO1995035375, the contents of each of which are herein incorporated by reference in their entirety).

Reslizumab blocks binding of human IL-5 to the IL-5 receptor complex expressed on the cell surface of eosinophils, inhibiting eosinophilic inflammation. This was also demonstrated in preclinical models, where eosinophilic inflammation was inhibited by a single dose of reslizumab (1 mg/kg intraperitoneally) with long duration of action in allergic mice and Ascaris-responsive monkeys (as described in International Patent Publication WO1995035375, the contents of which are herein incorporated by reference in its entirety). While not wishing to be bound by theory, Reslizumab may function by reducing eosinophil accumulation, and reducing/reversing airway remodeling in eosinophilic asthma.

In one embodiment, the polynucleotides of the present invention may encode Reslizumab fragments or variants thereof described in International Patent Publication WO1995035375, the content of which is herein incorporated by reference in its entirety.

Certain sequences encoding the polynucleotide fragments, domains or heavy or light chains for Reslizumab are given in Table 41. The table is not an exhaustive list and any fragment or portion of the sequence which may be encoded in the polynucleotides of the invention.

TABLE 41
Table of Reslizumab Sequences

| | | | SEQ |
|---|---|---|---|
| | | | ID |
| Description | Sequence | Source | NO. |
| CDR grafted | MSVPTQVLGLLLLWLTDARCDIQMTQSPSSL | From | 181 |
| anti-IL-5 | SASVGDRVTITCLASEGISSYLAWYQQKPGK | WO9535375, | |
| light chain | APKLLIYGANSLQTGVPSRFSGSGSATDYTLT | FIG. 5 | |
| | ISSLQPEDFATYYCQQSYKFPNTFGQGTKVE | | |
| | VKR | | |
| CDR grafted | MGWSCIILFLVATATGVHSEVQLVESGGGLV | From | 182 |
| anti-IL-5 | QPGGSLRLSCAVSGLSLTSNSVNWIRQAPGK | WO9535375, | |
| heavy chain | GLEWVGLIWSNGDTDYNSAIKSRFTISRDTS | FIG. 6 | |
| | KSTVYLQMNSLRAEDTAVYYCAREYYGYFD | | |
| | YWGQGTLVTVSS | | |

In one embodiment, the polynucleotides described herein may encode Reslizumab, a fragment or variant thereof and the polynucleotides may be used for the treatment of severe asthma. As a non-limiting example, a subject whose symptoms were not controlled by inhaled corticosteroids may use the polynucleotides described herein encoding Reslizumab, a fragment or variant thereof as treatment for severe asthma.

In one embodiment, the polynucleotides described herein may encode Reslizumab, a fragment or variant thereof and the polynucleotides may be used for the treatment of nasal oplyposis.

In one embodiment, polynucleotides encoding Reslizumab, fragments or variants thereof may be used to prevent, treat or manage an allergic and/or atopic response associated with elevated levels of eosinophils.

In one embodiment, the polynucleotides described herein may encode Reslizumab, a fragment or variant thereof and the polynucleotides may be used for the treatment of eosinophilic asthma.

In one embodiment, the polynucleotides described herein may encode Reslizumab, a fragment or variant thereof and the polynucleotides may be used for the treatment of inflammatory diseases with a marked eosinophilic component, such as, but not limited to, types of Hypereosinophilic Syndrome (HES).

In one embodiment, polynucleotides encoding Reslizumab, fragments or variants thereof may be used to prevent, treat or manage diseases with airway eosinophilia, eosinophilic asthma, eosinophilic COPD, eosinophilic bronchitis, and airway eosinophilia associated with viral infections, including, but not limited to, rhino virus.

In one embodiment, polynucleotides encoding Reslizumab, fragments or variants thereof may be used to prevent, treat or manage a disease with excess eosinophil production selected from the group consisting of atopic asthma, atopic dermatitis, allergic rhinitis, non-allergic rhinitis, asthma, severe asthma, chronic eosinophilic pneumonia, allergic bronchopulmonary aspergillosis, coeliac disease, Churg-Strauss syndrome, eosinophilic myalgia syndrome, hypereosinophilic syndrome, oedematous reactions including episodic angiodema, helminth infections, onchocercal dermatitis eosinophilic oesophagitis, eosinophilic gastritis, eosinophilic gastroenteritis, eosinophilic enteritis, eosinophilic colitis, nasal micropolyposis, nasal polyposis, aspirin intolerance asthma, obstructive sleep apnoe, chronic asthma, Crohn's disease, scleroderma and endomyocardial fibrosis.

In one embodiment polynucleotides encoding Reslizumab, fragments or variants thereof may be used to treat to prevent, treat or manage a disease with airway eosinophilia as determined by the measurement of indicators or symptoms of eosinophilia within certain parameters known in the art, including Asthma Symptom Score, Eosinophilic Cationic Protein Levels, Fractional Exhaled Nitric Oxide (FENO), Methacholine Challenge Test (MCT), circulating eosinophil count eosinophil count in induced sputum, or circulating basophil count as described in International Patent Publication WO2008143878, the content of which is herein incorporated by reference its entirety.

In one embodiment, polynucleotides encoding Reslizumab, fragments or variants thereof may be used in combination with a second an anti-asthma medication (corticosteroids, non-steroidal agents, beta agonists, leukotriene antagonists, xanthines, fluticasone, salmeterol, albuterol) which may be delivered by inhalation or other appropriate means.

In one embodiment, polynucleotides encoding Reslizumab, fragments or variants thereof may be administered in combination with an a further IL-5 antagonist, including but not limited to mepolizumab and reslizumab or benralizumab or antibodies described in International Patent Publication Nos. WO2008134724 and WO2009120927, the contents of each of which are incorporated herein by reference in its entirety, alone or in combination with a corticosteroid and/or a second anti-asthma medication.

### Benralizumab Parent Molecule or Antibody

In one embodiment, the polynucleotides of the present invention may encode Benralizumab, fragments or variants thereof.

Benralizumab, also known as MEDI-563, is a fully humanized IgG1 anti-human IL5-Rα antibody that binds to an epitope on IL-5Rα which is in close proximity to the IL-5 binding site and thus inhibits IL-5 receptor signaling originally developed by MedImmune. Benralizumab induces apoptosis through antibody-dependent cell-mediated cytotoxicity (ADCC) depleting eosinophils, a key target cell in inflammatory respiratory disease.

Benralizumab is also afucosylated. i.e., a fucose sugar residue in the CH2 region of the oligosaccharide core of human IgG1 is removed. This removal results in a 5- to 50-fold higher affinity to the main activating Fcγreceptor (human FcγRIIIa) expressed on natural killer (NK) cells, macrophages and neutrophils. Therefore, afucosylated benralizumab has an improved receptor-mediated effector function, resulting in an amplified eosinophil apoptosis in vitro via antibody-dependent cell-mediated cytotoxicity (ADCC) by more than 1000-fold over the parental antibody (Kolbeck et al., 2009 and Ferrara et al, 2011, the contents of each of which are incorporated herein by reference in their entirety).

Benralizumab is a monoclonal antibody that binds to a distinct epitope within the extracellular domain of recombinant human IL-5Rα, preventing receptor dimerization and subsequent inflammatory signaling (Ghazi et al, 2012). Benralizumab is afucosylated, and afucosylation is associated with enhanced ADCC. Benralizumab was found to induce apoptosis in eosinophils and basophils through ADCC (Kolbeck et al., 2009). While anti-IL-5 mAbs act by neutralizing the effects of IL-5, in contrast Benralizumab targets the effector cells, mainly eosinophils and basophils, and consequently actively depletes these cells. Indeed, in animal models, Benralizumab depleted peripheral blood eosinophils to less than the limit of detection (Ghazi et al, 2012, Busse et al, 2010, Kolbeck et al, 2009).

In one embodiment, the polynucleotides of the present invention may encode any of the Benralizumab sequences or fragments or variants thereof described in International Patent Publication WO2008143878, the content of which is herein incorporated by reference in its entirety.

Certain sequences encoding the polynucleotide fragments, domains or heavy or light chains for Benralizumab are given in Table 42. The table is not an exhaustive list and any fragment or portion of the sequence which may be encoded in the polynucleotides of the invention.

TABLE 42
Table of Benralizumab Sequences

| | | | SEQ |
|---|---|---|---|
| | | | ID |
| Description | Sequence | Source | NO. |
| Heavy chain | EVQLVQSGAEVKKPGASVKVSCKASGYTFTS | From | 183 |
| | YVIHWVRQRPGQGLAWMGYINPYNDGTKYN | WO2008143878 | |
| | ERFKGKVTITSDRSTSTVYMELSSLRSEDTAV | SEQ ID NO 4 | |
| | YLCGREGIRYYGLLGDYWGQGTLVTVSSAST | | |
| | KGPSVFPLAPSSKSTSGGTAALGCLVKDYFPE | | |
| | PVTVSWNSGALTSGVHTFPAVLQSSGLYSLSS | | |
| | VVTVPSSSLGTQTYICNVNHKPSNTKVDKKVE | | |
| | PKSCDKTHTCPPCPAPELLGGPSVFLFPPKPKD | | |
| | TLMISRTPEVTCVVVDVSHEDPEVKFNWYVD | | |
| | GVEVHNAKTKPREEQYNSTYRVVSVLTVLHQ | | |
| | DWLNGKEYKCKVSNKALPAPIEKTISKAKGQ | | |
| | PREPQVYTLPPSRDELTKNQVSLTCLVKGFYP | | |
| | SDIAVEWESNGQPENNYKTTPPVLDSDGSFFL | | |
| | YSKLTVDKSRWQQGNVFSCSVMHEALHNHY | | |
| | TQKSLSLSPGK | | |
| Heavy chain | EVQLVQSGAEVKKPGASVKVSCKASGYTFTS | From | 184 |
| | YVIHWVRQRPGQGLAWMGYINPYNDGTKYN | WO2008143878 | |
| | ERFKGKVTITSDRSTSTVYMELSSLRSEDTAV | SEQ ID | |
| | YLCGREGIRYYGLLGDYWGQGTLVTVSS | NO: 3 | |
| Light chain | DIQMTQSPSSLSASVGDRVTITCGTSEDIINYL | From | 185 |
| | NWYQQKPGKAPKLLIYHTSRLQSGVPSRFSGS | WO2008143878 | |
| | GSGTDFTLTISSLQPEDFATYYCQQGYTLPYTF | SEQ ID | |
| | GQGTKVEIKRTVAAPSVFIFPPSDEQLKSGTAS | NO: 2 | |
| | VVCLLNNFYPREAKVQWKVDNALQSGNSQE | | |
| | SVTEQDSKDSTYSLSSTLTLSKADYEKHKVYA | | |
| | CEVTHQGLSSPVTKSFNRGEC | | |
| Light chain | DIQMTQSPSSLSASVGDRVTITCGTSEDIINYL | From | 186 |
| | NWYQQKPGKAPKLLIYHTSRLQSGVPSRFSGS | WO2008143878 | |
| | GSGTDFTLTISSLQPEDFATYYCQQGYTLPYTF | SEQ ID | |
| | GQGTKVEIK | NO: 1 | |

In one embodiment, the polynucleotides described herein may encode Benralizumab, a fragment or variant thereof and the polynucleotides may be used to reduce the accumulation of eosinophils. Eosinophils are not normally present in healthy lung tissue, but accumulate in the lung as a result of inflammatory processes in eosinophilic asthma, a subtype of asthma characterized by increased blood or sputum eosinophils. Increased levels of eosinophils are correlated with the severity and frequency of asthma exacerbations.

In one embodiment, polynucleotides encoding Benralizumab, fragments or variants thereof may be reduce the number of eosinophils or eosinophil precursors by at least about 10%, at least about 20%, at least about 30%, at least about 40%, at least about 50%, at least about 60%, at least about 70%, at least about 80%; at least about 90%, at least about 95% or at least about 99%. In a specific embodiment, the polynucleotides of the invention reduce the number of eosinophils below the limit of detection.

In one embodiment, the polynucleotides described herein may encode Benralizumab, a fragment or variant thereof and the polynucleotides may be used to reduce circulating eosinophil levels (Busse et al, 2010).

In one embodiment, the polynucleotides described herein may encode Benralizumab, a fragment or variant thereof and the polynucleotides may be used to treat chronic obstructive pulmonary disease and uncontrolled asthma.

In one embodiment, the polynucleotides described herein may encode Benralizumab, a fragment or variant thereof and the polynucleotides may be used to treat hypereosinophilic syndrome (HES), eosinophilic esophagitis, nasal polyposis or Churg-Strauss syndrome.

In one embodiment, polynucleotides encoding Benralizumab, fragments or variants thereof may be used to prevent or treat or manage diseases with airway eosinophilia, eosinophilic asthma, eosinophilic, eosinophilic bronchitis, and airway eosinophilia associated with viral infections, including, but not limited to, rhino virus.

### Sirukumab Parent Molecule or Antibody

In one embodiment, the polynucleotides of the present invention may encode Sirukumab, fragments or variants thereof.

Sirukumab, also known as CNTO 136 and BA003, is a human IgG1kappa monoclonal antibody that targets the cytokine interleukin 6 (IL-6) developed by Janssen Biologics (Ireland) and co-developed by GlaxoSmithKline (GSK) and Janssen Biologics. IL-6 has many effects and plays an important role in the host defense against pathogens. However, when IL-6 production is dysregulated, IL-6 promotes the development of chronic autoimmune and inflammatory diseases.

Sirukumab is a human engineered antibody with fully human frameworks and constant regions (CL, CH domains (e.g., CH1, CH2, CH3), and hinge), and CDRs derived from the frameworks of a mouse antibody specific to IL-6. Sequences and development of Sirukumab are disclosed in U.S. Pat. No. 7,833,755, the content of which is herein incorporated by reference in its entirety. In one embodiment, the polynucleotides described herein may encode any of the Sirukumab or IL-6 antibody sequences, fragments or variants thereof described in U.S. Pat. No. 7,833,755, the content of which is herein incorporated by reference in its entirety.

Certain sequences encoding the polynucleotide fragments, domains or heavy or light chains for Sirukumab are given in Table 43. The table is not an exhaustive list and any fragment or portion of the sequence which may be encoded in the polynucleotides of the invention.

TABLE 43
Table of Sirukumab Sequences

| | | | SEQ |
|---|---|---|---|
| | | | ID |
| Description | Sequence | Source | NO. |
| Heavy chain | EVQLVESGGGLVQPGGSLRLSCAASGFTF | | 187 |
| | SPFAMSWVRQAPGKGLEWVAKISPGGSW | | |
| | TYYSDTVTGRFTISRDNAKNSLYLQMNSL | | |
| | RAEDTAVYYCARQLWGYYALDIWGQGT | | |
| | TVTVSSASTKGPSVFPLAPSSKSTSGGTAA | | |
| | LGCLVKDYFPEPVTVSWNSGALTSGVHTF | | |
| | PAVLQSSGLYSLSSVVTVPSSSLGTQTYIC | | |
| | NVNHKPSNTKVDKKVEPKSCDKTHTCPPC | | |
| | PAPELLGGPSVFLFPPKPKDTLMISRTPEVT | | |
| | CVVVDVSHEDPEVKFNWYVDGVEVHNA | | |
| | KTKPREEQYNSTYRVVSVLTVLHQDWLN | | |
| | GKEYKCKVSNKALPAPIEKTISKAKGQPR | | |
| | EPQVYTLPPSRDELTKNQVSLTCLVKGFY | | |
| | PSDIAVEWESNGQPENNYKTTPPVLDSDG | | |
| | SFFLYSKLTVDKSRWQQGNVFSCSVMHE | | |
| | ALHNHYTQKSLSLSPGK | | |
| Light chain | EIVLTQSPATLSLSPGERATLSCSASISVSY | | 188 |
| | MYWYQQKPGQAPRLLIYDMSNLASGIPA | | |
| | RFSGSGSGTDFTLTISSLEPEDFAVYYCMQ | | |
| | WSGYPYTFGGGTKVEIKRTVAAPSVFIFPP | | |
| | SDEQLKSGTASVVCLLNNFYPREAKVQW | | |
| | KVDNALQSGNSQESVTEQDSKDSTYSLSS | | |
| | TLTLSKADYEKHKVYACEVTHQGLSSPVT | | |
| | KSFNRGEC | | |
| Light chain | EIVLTQSPATLSLSPGERATLSCSASISVSY | U.S. Pat. No. | 189 |
| variable | MYWYQQKPGQAPRLLIYDMSNLASGIPA | 7,833,755 | |
| region | RFSGSGSGTDFTLTISSLEPEDFAVYYCMQ | SEQ ID NO: 97 | |
| | WSGYPYTFGGGTKVEIK | | |
| Heavy chain | EVQLVESGGGLVQPGGSLRLSCAASGFTF | U.S. Pat. No. | 190 |
| variable | SPFAMSWVRQAPGKGLEWVAKISPGGSW | 7,833,755 | |
| region | TYYSDTVTGRFTISRDNAKNSLYLQMNSL | SEQ ID NO: 99 | |
| | RAEDTAVYYCARQLWGYYALDIWGQGT | | |
| | TVTVSS | | |
| Light chain | SASISVSYMY | U.S. Pat. No. | 191 |
| CDR1 | | 7,833,755 | |
| | | SEQ ID NO: 3 | |
| Light chain | DMSNLAS | U.S. Pat. No. | 192 |
| CDR2 | | 7,833,755 | |
| | | SEQ ID NO: 21 | |
| Light chain | MQWSGYPYT | U.S. Pat. No. | 193 |
| CDR3 | | 7,833,755 | |
| | | SEQ ID NO: 29 | |
| Heavy chain | GFTFSPFAMS | U.S. Pat. No. | 194 |
| CDR1 | | 7,833,755 | |
| | | SEQ ID NO: 39 | |
| Heavy chain | KISPGGSWTYYSDTVTG | U.S. Pat. No. | 195 |
| CDR2 | | 7,833,755 | |
| | | SEQ ID NO: 59 | |
| Heavy chain | QLWGYYALDI | U.S. Pat. No. | 196 |
| CDR3 | | 7,833,755 | |
| | | SEQ ID NO: 89 | |

In one embodiment, the polynucleotides described herein may encode Sirukumab, a fragment or variant thereof and the polynucleotides may be used to treat and/or prevent Rheumatoid arthritis. As a non-limiting example, the polynucleotides described herein may encode Sirukumab, a fragment or variant thereof and the polynucleotides may be used to improve Rheumatoid arthritis signs and symptoms in a subject. The subject may have active Rheumatoid arthritis and may be refractory or intolerant to anti-TNF-alpha agents.

In one embodiment, the polynucleotides described herein may encode Sirukumab, a fragment or variant thereof and the polynucleotides may be used to prevent, treat or manage an autoimmune disease, which is associated with elevated levels of the cytokine IL-6.

In one embodiment, the polynucleotides described herein may encode Sirukumab, a fragment or variant thereof and the polynucleotides may be used to prevent, treat or manage an autoimmune disease, such as rheumatoid arthritis in combination with DMARDs, such as methotrexate.

In one embodiment, polynucleotides encoding Sirukumab, fragments or variants thereof may be used in combination with one or more additional treatment regimen to prevent, treat or manage an autoimmune disease, including but not limited to nonsteroidal anti-inflammatory drugs (NSAIDs), e.g. ibuprofen, corticosteroid medications, including but not limited to prednisone, disease-modifying antirheumatic drugs (DMARDs), including but not limited to methotrexate (TREXALL®), leflunomide (ARAVA®), hydroxychloroquine (PLAQUENIL®) and sulfasalazine (AZULFIDINE®), immunosuppressants, including but not limited to azathioprine (IMURAN®) and cyclosporine, and TNF-alpha inhibitors, including etanercept (ENBREL®), infliximab (REMICADE®), adalimumab (HUMIRA®), golimumab (SIMPONI®) and certolizumab (CIMZIA®) and other drugs targeting a variety of processes involved with inflammation. These other drugs include but are not limited to anakinra (KINETERET®), abatacept (ORENCIA®), rituximab (RITUXAN®), tocilizumab (ACTEMRA®) and tofacitinib (XELJANZ®).

In one embodiment, the polynucleotides described herein may encode Sirukumab, a fragment or variant thereof and the polynucleotides may be used alone in combination with one or more additional treatment regime as part of a maintenance regimen and/or at start of relapse for an autoimmune disease, such as, but not limited to, Rheumatoid Arthritis.

### Siltuximab Parent Molecule or Antibody

In one embodiment, the polynucleotides of the present invention may encode Siltuximab, fragments or variants thereof.

Siltuximab, also known as cCLB-8, is a chimeric monoclonal antibody with high affinity to an inhibiting and/or neutralizing epitope of human interleukin 6 (IL-6) developed by Janssen Research & Development LLC, a pharmaceutical company of Johnson & Johnson. IL-6 has many effects and plays an important role in the host defense against pathogens. However, when IL-6 production is dysregulated, IL-6 promotes the development of chronic autoimmune and inflammatory diseases.

Siltuximab light chains and heavy chains comprise at least part of a human constant region and at least part of a variable region, e.g. the CDR regions are derived from the murine CLB8 monoclonal antibody, as described in U.S. Pat. No. 7,955,597 the content of which is herein incorporated by reference in its entirety. Siltuximab sequences are disclosed in U.S. Pat. No. 7,955,597 the content of which is herein incorporated by reference in its entirety. In one embodiment, the polynucleotides described herein may encode any of the Siltuximab or IL-6 antibody sequences, fragments or variants thereof described in U.S. Pat. No. 7,955,597, the content of which is herein incorporated by reference in its entirety.

Certain sequences encoding the polynucleotide fragments, domains or heavy or light chains for Siltuximab are given in Table 44. The table is not an exhaustive list and any fragment or portion of the sequence which may be encoded in the polynucleotides of the invention.

TABLE 44
Table of Siltuximab Sequences

| | | | SEQ |
|---|---|---|---|
| | | | ID |
| Description | Sequence | Source | NO. |
| Heavy chain | EVQLVESGGKLLKPGGSLKLSCAASGFTF | | 197 |
| | SSFAMSWFRQSPEKRLEWVAEISSGGSYT | | |
| | YYPDTVTGRFTISRDNAKNTLYLEMSSLR | | |
| | SEDTAMYYCARGLWGYYALDYWGQGTS | | |
| | VTVSSASTKGPSVFPLAPSSKSTSGGTAAL | | |
| | GCLVKDYFPEPVTVSWNSGALTSGVHTFP | | |
| | AVLQSSGLYSLSSVVTVPSSSLGTQTYICN | | |
| | VNHKPSNTKVDKKVEPKSCDKTHTCPPCP | | |
| | APELLGGPSVFLFPPKPKDTLMISRTPEVT | | |
| | CVVVDVSHEDPEVKFNWYVDGVEVHNA | | |
| | KTKPREEQYNSTYRVVSVLTVLHQDWLN | | |
| | GKEYKCKVSNKALPAPIEKTISKAKGQPR | | |
| | EPQVYTLPPSRDELTKNQVSLTCLVKGFY | | |
| | PSDIAVEWESNGQPENNYKTTPPVLDSDG | | |
| | SFFLYSKLTVDKSRWQQGNVFSCSVMHE | | |
| | ALHNHYTQKSLSLSPGK | | |
| Light chain | QIVLIQSPAIMSASPGEKVTMTCSASSSVS | | 198 |
| | YMYWYQQKPGSSPRLLIYDTSNLASGVPV | | |
| | RFSGSGSGTSYSLTISRMEAEDAATYYCQ | | |
| | QWSGYPYTFGGGTKLEIKRTVAAPSVFIFP | | |
| | PSDEQLKSGTASVVCLLNNFYPREAKVQ | | |
| | WKVDNALQSGNSQESVTEQDSKDSTYSLS | | |
| | STLTLSKADYEKHKVYACEVTHQGLSSPV | | |
| | TKSFNRGEC | | |
| Heavy chain | EVQLVESGGKLLKPGGSLKLSCAASGFTF | From U.S. Pat. | 199 |
| variable | SSFAMSWFRQSPEKRLEWVAEISSGGSYT | No. 7,955,597 | |
| region | YYPDTVTGRFTISRDNAKNTLYLEMSSLR | SEQ ID NO: 7 | |
| | SEDTAMYYCARGLWGYYALDYWGQGTS | | |
| | VTVSS | | |
| Light chain | QIVLIQSPAIMSASPGEKVTMTCSASSSVS | From U.S. Pat. | 200 |
| variable | YMYWYQQKPGSSPRLLIYDTSNLASGVPV | No. 7,955,597 | |
| region | RFSGSGSGTSYSLTISRMEAEDAATYYCQ | SEQ ID NO: 8 | |
| | QWSGYPYTFGGGTKLEIK | | |
| Heavy chain | SFAMS | From U.S. Pat. | 201 |
| CDR1 | | No. 7,955,597 | |
| | | SEQ ID NO: 1 | |
| Heavy chain | EISSGGSYTYYPDTVTG | From U.S. Pat. | 202 |
| CDR2 | | No. 7,955,597 | |
| | | SEQ ID NO: 2 | |
| Heavy chain | GLWGYYALDY | From U.S. Pat. | 203 |
| CDR3 | | No. 7,955,597 | |
| | | SEQ ID NO: 3 | |
| Light chain | SASSSVSYMY | From U.S. Pat. | 204 |
| CDR1 | | No. 7,955,597 | |
| | | SEQ ID NO: 4 | |
| Light chain | DTSNLAS | From U.S. Pat. | 205 |
| CDR2 | | No. 7,955,597 | |
| | | SEQ ID NO: 5 | |
| Light chain | QQWSGYPYT | From U.S. Pat. | 206 |
| CDR3 | | No. 7,955,597 | |
| | | SEQ ID NO: 6 | |

In one embodiment, the polynucleotides described herein may encode Siltuximab, a fragment or variant thereof and the polynucleotides may be used to treat and/or prevent Castleman's disease. Castleman's disease is a disorder that affects lymph nodes and related tissues, the cause of which is unknown, although an individual's immune function may play a role. Infection with HIV is a risk factor, and Castleman's disease is much more common in people who are HIV positive. Two main forms exist: localized (localized to a single group of lymph nodes) and multicentric. Multicentric Castleman's disease (MCD) involves multiple groups of lymph nodes and can also affect other organs with lymphoid tissue. MCD is often associated serious infections, fevers, weight loss, loss of appetite, vomiting, fatigue, night sweats, and nerve damage, anemia and high levels of antibodies in the blood, and enlarged liver and spleen. MCD weakens the immune system, making infections serious and often fatal. 20% of people with this disease eventually develop lymphoma.

In one embodiment, the polynucleotides described herein may encode Siltuximab, a fragment or variant thereof and the polynucleotides may be used to treat and/or prevent localized Castleman's disease.

In one embodiment, the polynucleotides described herein may encode Siltuximab, a fragment or variant thereof and the polynucleotides may be used to treat and/or prevent multicentric Castleman's disease.

In one embodiment, the polynucleotides described herein may encode Siltuximab, a fragment or variant thereof and the polynucleotides may be used to suppress the C-reactive protein (CRP).

In one embodiment, the polynucleotides described herein may encode Siltuximab, a fragment or variant thereof and the polynucleotides may be used to may be used to prevent, treat or manage an autoimmune disease.

### Sarilumab Parent Molecule or Antibody

In one embodiment, the polynucleotides of the present invention may encode Sarilumab, fragments or variants thereof.

Sarilumab, also known as REGN88 or SAR153191, is a fully-human IgG1kappa monoclonal antibody directed against the IL-6 receptor (IL-6R) created using Regeneron's VELOCIMMUNE® antibody technology and in development by Sanofi and Regeneron. Sarilumab is an inhibitor of IL-6 signaling, which binds with high affinity to the IL-6 receptor. It blocks the binding of IL-6 to its receptor and interrupts the resultant cytokine-mediated inflammatory signaling.

Sarilumab was developed using by Regeneron's using a mouse capable of producing making antibodies with fully human variable regions, which are then combined with the desired human constant region. Sequences of Sarilumab are disclosed in US Patent Publication US20130149310, the content of which is herein incorporated by reference in its entirety. In one embodiment, the polynucleotides described herein may encode any of the Sarilumab or IL-6 antibody sequences, fragments or variants thereof described in US Patent Publication US20130149310, the content of which is herein incorporated by reference in its entirety.

Certain sequences encoding the polynucleotide fragments, domains or heavy or light chains for Sarilumab are given in Table 45. The table is not an exhaustive list and any fragment or portion of the sequence which may be encoded in the polynucleotides of the invention.

TABLE 45
Table of Sarilumab Sequences

| | | | SEQ |
|---|---|---|---|
| | | | ID |
| Description | Sequence | Source | NO. |
| Heavy chain | EVQLVESGGGLVQPGRSLRLSCAASRFTF | | 207 |
| | DDYAMHWVRQAPGKGLEWVSGISWNSG | | |
| | RIGYADSVKGRFTISRDNAENSLFLQMNG | | |
| | LRAEDTALYYCAKGRDSFDIWGQGTMVT | | |
| | VSSASTKGPSVFPLAPSSKSTSGGTAALGC | | |
| | LVKDYFPEPVTVSWNSGALTSGVHTFPAV | | |
| | LQSSGLYSLSSVVTVPSSSLGTQTYICNVN | | |
| | HKPSNTKVDKKVEPKSCDKTHTCPPCPAP | | |
| | ELLGGPSVFLFPPKPKDTLMISRTPEVTCV | | |
| | VVDVSHEDPEVKFNWYVDGVEVHNAKT | | |
| | KPREEQYNSTYRVVSVLTVLHQDWLNGK | | |
| | EYKCKVSNKALPAPIEKTISKAKGQPREPQ | | |
| | VYTLPPSRDELTKNQVSLTCLVKGFYPSDI | | |
| | AVEWESNGQPENNYKTTPPVLDSDGSFFL | | |
| | YSKLTVDKSRWQQGNVFSCSVMHEALHN | | |
| | HYTQKSLSLSPGK | | |
| Light chain | DIQMTQSPSSVSASVGDRVTITCRASQGIS | | 208 |
| | SWLAWYQQKPGKAPKLLIYGASSLESGVP | | |
| | SRFSGSGSGTDFTLTISSLQPEDFASYYCQ | | |
| | QANSFPYTFGQGTKLEIKRTVAAPSVFIFPP | | |
| | SDEQLKSGTASVVCLLNNFYPREAKVQW | | |
| | KVDNALQSGNSQESVTEQDSKDSTYSLSS | | |
| | TLTLSKADYEKHKVYACEVTHQGLSSPVT | | |
| | KSFNRGEC | | |
| Heavy chain | EVQLVESGGGLVQPGRSLRLSCAASRFTF | From U.S. Pat. | 209 |
| variable | DDYAMHWVRQAPGKGLEWVSGISWNSG | Publication | |
| region | RIGYADSVKGRFTISRDNAENSLFLQMNG | 20130149310 | |
| | LRAEDTALYYCAKGRDSFDIWGQGTMVT | SEQ ID NO: 2 | |
| | VSS | | |
| Light chain | DIQMTQSPSSVSASVGDRVTITCRASQGIS | From U.S. Pat. | 210 |
| variable | SWLAWYQQKPGKAPKLLIYGASSLESGVP | Publication | |
| region | SRFSGSGSGTDFTLTISSLQPEDFASYYCQ | 20130149310 | |
| | QANSFPYTFGQGTKLEIK | SEQ ID NO: 3 | |

In one embodiment, the polynucleotides described herein may encode Sarilumab, a fragment or variant thereof and the polynucleotides may be used to treat and/or prevent Rheumatoid arthritis. Rheumatoid arthritis is an autoimmune disorder that results in chronic inflammation in the small joints in the hands and feet, causing a painful swelling that can eventually result in bone erosion and joint deformity.

In one embodiment, polynucleotides encoding Sarilumab, fragments or variants thereof may be used to prevent, treat or manage an autoimmune disease associated with elevated levels of the cytokine IL-6.

In one embodiment, polynucleotides encoding Sarilumab, fragments or variants thereof may be used to prevent, treat or manage an autoimmune disease, such as rheumatoid arthritis (RA), including cases where the patient has been previously treated with a TNF antagonist and/or was intolerant to an TNF antagonist. In another embodiment the subject was previously ineffectively treated for RA by administering DMARDS, such as methotrexate, leflunomide, sulfasalazine and/or hydroxychloroquine.

In one embodiment, polynucleotides encoding Sarilumab, fragments or variants thereof may be used to prevent, treat or manage an autoimmune disease, such as rheumatoid arthritis (RA) in combination with DMARDs, such as methotrexate.

In one embodiment, polynucleotides encoding Sarilumab, fragments or variants thereof may be used in a method of treatment wherein the subject achieves a 20%, 50% or 70% improvement in the American College of Rheumatology core set disease index (ACR20, 50 or 70) after 12 weeks of treatment.

In one embodiment, polynucleotides encoding Sarilumab, fragments or variants thereof may be used alone in combination with one or more additional treatment regime as part of a maintenance regimen and/or at start of relapse for an autoimmune disease, such as, but not limited to, RA.

### Lebrikizumab Parent Molecule or Antibody

In one embodiment, the polynucleotides of the present invention may encode Lebrikizumab, fragments or variants thereof.

Lebrikizumab, also known as RG3637, MILR1444A and TNX-650, is a humanized IgG4k monoclonal antibody directed against IL-13 developed by Roche (Genentech). Lebrikizumab specifically blocks the action of interleukin-13 (IL-13), a cytokine that contributes to airway inflammation and asthma in some patients. Lebrikizumab has been altered by a single point mutation in the hinge region to increase the stability of the molecule (Aalberse and Schuurman, Immunology 2002, 105:9-19, the contents of which are herein incorporated by reference in its entirety).

Lebrikizumab sequences are described in International Patent Publication WO2013066866, the contents of which is incorporated herein by reference in its entirety. Additional anti-IL13 antibodies are further described in International Patent Publication WO2005062967, the contents of which is herein incorporated by reference in its entirety.

In one embodiment, the polynucleotides described herein may encode any of the Lebrikizumab or anti-IL13 antibody sequences, fragments or variants thereof described in International Patent Publication WO2013066866, the contents of which is incorporated herein by reference in its entirety.

In one embodiment, the polynucleotides described herein may encode any of the Lebrikizumab or anti-IL13 antibody sequences, fragments or variants thereof described in International Patent Publication WO2005062967, the contents of which is incorporated herein by reference in its entirety.

Certain sequences encoding the polynucleotide fragments, domains or heavy or light chains for Lebrikizumab are given in Table 46. The table is not an exhaustive list and any fragment or portion of the sequence which may be encoded in the polynucleotides of the invention.

TABLE 46
Table of Lebrikizumab Sequences

| | | | SEQ |
|---|---|---|---|
| | | | ID |
| Description | Sequence | Source | NO. |
| Heavy chain | AYSVN | From | 211 |
| CDR1 | | WO2013066866 | |
| | | SEQ ID NO: 1 | |
| Heavy chain | MIWGDGKIVYNSALKS | From | 212 |
| CDR2 | | WO2013066866 | |
| | | SEQ ID NO: 2 | |
| Heavy chain | DGYYPYAMDN | From | 213 |
| CDR3 | | WO2013066866 | |
| | | SEQ ID NO: 3 | |
| Light chain | RASKSVDSYGNSFMH | From | 214 |
| CDR1 | | WO2013066866 | |
| | | SEQ ID NO: 4 | |
| Light chain | LASNLES | From | 215 |
| CDR2 | | WO2013066866 | |
| | | SEQ ID NO: 5 | |
| Light chain | QQNNEDPRT | From | 216 |
| CDR3 | | WO2013066866 | |
| | | SEQ ID NO: 6 | |
| Heavy chain | VTLRESGPALVKPTQTLTLTCTVSGFSLSA | From | 217 |
| variable | YSVNWIRQPPGKALEWLAMIWGDGKIVY | WO2013066866 | |
| region | NSALKSRLTISKDTSKNQVVLTMTNMDPV | SEQ ID NO: 7 | |
| | DTATYYCAGDGYYPYAMDNWGQGSLVT | | |
| | VSS | | |
| Heavy chain | QVTLRESGPALVKPTQTLTLTCTVSGFSLS | From | 218 |
| variable | AYSVNWIRQPPGKALEWLAMIWGDGKIV | WO2013066866 | |
| region | YNSALKSRLTISKDTSKNQVVLTMTNMDP | SEQ ID NO: 8 | |
| | VDTATYYCAGDGYYPYAMDNWGQGSLV | | |
| | TVSS | | |
| Light chain | DIVMTQSPDSLSVSLGERATINCRASKSVD | From | 219 |
| variable | SYGNSFMHWYQQKPGQPPKLLIYLASNLE | WO2013066866 | |
| region | SGVPDRFSGSGSGTDFTLTISSLQAEDVAV | SEQ ID NO: 9 | |
| | YYCQQNNEDPRTFGGGTKVEIKR | | |
| Heavy chain | VTLRESGPALVKPTQTLTLTCTVSGFSLSA | From | 220 |
| | YSVNWIRQPPGKALEWLAMIWGDGKIVY | WO2013066866 | |
| | NSALKSRLTISKDTSKNQVVLTMTNMDPV | SEQ ID NO: 10 | |
| | DTATYYCAGDGYYPYAMDNWGQGSLVT | | |
| | VSSASTKGPSVFPLAPCSRSTSESTAALGC | | |
| | LVKDYFPEPVTVSWNSGALTSGVHTFPAV | | |
| | LQSSGLYSLSSVVTVPSSSLGTKTYTCNVD | | |
| | HKPSNTKVDKRVESKYGPPCPPCPAPEFL | | |
| | GGPSVFLFPPKPKDTLMISRTPEVTCVVVD | | |
| | VSQEDPEVQFNWYVDGVEVHNAKTKPRE | | |
| | EQFNSTYRVVSVLTVLHQDWLNGKEYKC | | |
| | KVSNKGLPSSIEKTISKAKGQPREPQVYTL | | |
| | PPSQEEMTKNQVSLTCLVKGFYPSDIAVE | | |
| | WESNGQPENNYKTTPPVLDSDGSFFLYSR | | |
| | LTVDKSRWQEGNVFSCSVMHEALHNHYT | | |
| | QKSLSLSLG | | |
| Heavy chain | QVTLRESGPALVKPTQTLTLTCTVSGFSLS | From | 221 |
| | AYSVNWIRQPPGKALEWLAMIWGDGKIV | WO2013066866 | |
| | YNSALKSRLTISKDTSKNQVVLTMTNMDP | SEQ ID NO: 11 | |
| | VDTATYYCAGDGYYPYAMDNWGQGSLV | | |
| | TVSSASTKGPSVFPLAPCSRSTSESTAALG | | |
| | CLVKDYFPEPVTVSWNSGALTSGVHTFPA | | |
| | VLQSSGLYSLSSVVTVPSSSLGTKTYTCNV | | |
| | DHKPSNTKVDKRVESKYGPPCPPCPAPEF | | |
| | LGGPSVFLFPPKPKDTLMISRTPEVTCVVV | | |
| | DVSQEDPEVQFNWYVDGVEVHNAKTKPR | | |
| | EEQFNSTYRVVSVLTVLHQDWLNGKEYK | | |
| | CKVSNKGLPSSIEKTISKAKGQPREPQVYT | | |
| | LPPSQEEMTKNQVSLTCLVKGFYPSDIAVE | | |
| | WESNGQPENNYKTTPPVLDSDGSFFLYSR | | |
| | LTVDKSRWQEGNVFSCSVMHEALHNHYT | | |
| | QKSLSLSLG | | |
| Heavy chain | VTLRESGPALVKPTQTLTLTCTVSGFSLSA | From | 222 |
| | YSVNWIRQPPGKALEWLAMIWGDGKIVY | WO2013066866 | |
| | NSALKSRLTISKDTSKNQVVLTMTNMDPV | SEQ ID NO: 12 | |
| | DTATYYCAGDGYYPYAMDNWGQGSLVT | | |
| | VSSASTKGPSVFPLAPCSRSTSESTAALGC | | |
| | LVKDYFPEPVTVSWNSGALTSGVHTFPAV | | |
| | LQSSGLYSLSSVVTVPSSSLGTKTYTCNVD | | |
| | HKPSNTKVDKRVESKYGPPCPPCPAPEFL | | |
| | GGPSVFLFPPKPKDTLMISRTPEVTCVVVD | | |
| | VSQEDPEVQFNWYVDGVEVHNAKTKPRE | | |
| | EQFNSTYRVVSVLTVLHQDWLNGKEYKC | | |
| | KVSNKGLPSSIEKTISKAKGQPREPQVYTL | | |
| | PPSQEEMTKNQVSLTCLVKGFYPSDIAVE | | |
| | WESNGQPENNYKTTPPVLDSDGSFFLYSR | | |
| | LTVDKSRWQEGNVFSCSVMHEALHNHYT | | |
| | QKSLSLSLGK | | |
| Heavy chain | QVTLRESGPALVKPTQTLTLTCTVSGFSLS | From | 223 |
| | AYSVNWIRQPPGKALEWLAMIWGDGKIV | WO2013066866 | |
| | YNSALKSRLTISKDTSKNQVVLTMTNMDP | SEQ ID NO: 13 | |
| | VDTATYYCAGDGYYPYAMDNWGQGSLV | | |
| | TVSSASTKGPSVFPLAPCSRSTSESTAALG | | |
| | CLVKDYFPEPVTVSWNSGALTSGVHTFPA | | |
| | VLQSSGLYSLSSVVTVPSSSLGTKTYTCNV | | |
| | DHKPSNTKVDKRVESKYGPPCPPCPAPEF | | |
| | LGGPSVFLFPPKPKDTLMISRTPEVTCVVV | | |
| | DVSQEDPEVQFNWYVDGVEVHNAKTKPR | | |
| | EEQFNSTYRVVSVLTVLHQDWLNGKEYK | | |
| | CKVSNKGLPSSIEKTISKAKGQPREPQVYT | | |
| | LPPSQEEMTKNQVSLTCLVKGFYPSDIAVE | | |
| | WESNGQPENNYKTTPPVLDSDGSFFLYSR | | |
| | LTVDKSRWQEGNVFSCSVMHEALHNHYT | | |
| | QKSLSLSLGK | | |
| Light chain | DIVMTQSPDSLSVSLGERATINCRASKSVD | From | 224 |
| | SYGNSFMHWYQQKPGQPPKLLIYLASNLE | WO2013066866 | |
| | SGVPDRFSGSGSGTDFTLTISSLQAEDVAV | SEQ ID NO: 14 | |
| | YYCQQNNEDPRTFGGGTKVEIKRTVAAPS | | |
| | VFIFPPSDEQLKSGTASVVCLLNNFYPREA | | |
| | KVQWKVDNALQSGNSQESVTEQDSKDST | | |
| | YSLSSTLTLSKADYEKHKVYACEVTHQGL | | |
| | SSPVTKSFNRGEC | | |

In one embodiment, the polynucleotides described herein may encode Lebrikizumab, a fragment or variant thereof and the polynucleotides may be used to treat and/or prevent asthma. As a non-limiting example, the polynucleotides described herein may encode Lebrikizumab, a fragment or variant thereof and the polynucleotides may be used to reduce asthma attack rates. As another non-limiting example, the polynucleotides described herein may encode Lebrikizumab, a fragment or variant thereof and the polynucleotides may be used to improve lung function in subjects with sever uncontrolled asthma.

In one embodiment, the polynucleotides described herein may encode Lebrikizumab, a fragment or variant thereof and the polynucleotides may be used to treat and/or prevent idiopathic pulmonary fibrosis (IPF). IPF is a life-threatening scarring or thickening of the lungs without a known cause.

In one embodiment, polynucleotides encoding Lebrikizumab, fragments or variants thereof may be used to prevent or treat conditions, characterized by abnormal or excess expression of IL-13, such as, but not limited to, asthma.

### Secukinumab Parent Molecule or Antibody

In one embodiment, the polynucleotides of the present invention may encode Secukinumab, fragments or variants thereof.

Secukinumab, also known as AIN457, is a fully human IgG1 monoclonal antibody that binds with high affinity and selectivity to human IL-17A developed by Novartis Pharmaceuticals. IL-17A is a key pro-inflammatory cytokine produced by a subset of T helper cells called Th17 cells and forms homo or heterodimers with IL-17F. IL-17 cytokines play key regulatory roles in host defense and inflammatory diseases. IL-17A exerts its biological effect by promoting the release of pro-inflammatory cytokines and chemokines, thereby attracting neutrophils and macrophages to the inflammation site.

Development and sequences of Secukinumab are described in International Patent Publication WO2006013107, the contents of which is herein incorporated by reference in its entirety. In one embodiment, the polynucleotides described herein may encode any of the IL-17A antibodies or fragments disclosed in International Patent Publication WO2006013107, the contents of which is herein incorporated by reference in its entirety.

Certain sequences encoding the polynucleotide fragments, domains or heavy or light chains for Secukinumab are given in Table 47. The table is not an exhaustive list and any fragment or portion of the sequence which may be encoded in the polynucleotides of the invention.

TABLE 47
Table of Secukinumab Sequences

| | | | SEQ |
|---|---|---|---|
| | | | ID |
| Description | Sequence | Source | NO. |
| Heavy chain | EVQLVESGGGLVQPGGSLRLSCAASGFTF | | 225 |
| | SNYWMNWVRQAPGKGLEWVAAINQDGS | | |
| | EKYYVGSVKGRFTISRDNAKNSLYLQMNS | | |
| | LRVEDTAVYYCVRDYYDILTDYYIHYWY | | |
| | FDLWGRGTLVTVSSASTKGPSVFPLAPSSK | | |
| | STSGGTAALGCLVKDYFPEPVTVSWNSGA | | |
| | LTSGVHTFPAVLQSSGLYSLSSVVTVPSSS | | |
| | LGTQTYICNVNHKPSNTKVDKRVEPKSCD | | |
| | KTHTCPPCPAPELLGGPSVFLFPPKPKDTL | | |
| | MISRTPEVTCVVVDVSHEDPEVKFNWYV | | |
| | DGVEVHNAKTKPREEQYNSTYRVVSVLT | | |
| | VLHQDWLNGKEYKCKVSNKALPAPIEKTI | | |
| | SKAKGQPREPQVYTLPPSREEMTKNQVSL | | |
| | TCLVKGFYPSDIAVEWESNGQPENNYKTT | | |
| | PPVLDSDGSFFLYSKLTVDKSRWQQGNVF | | |
| | SCSVMHEALHNHYTQKSLSLSPGK | | |
| Light chain | EIVLTQSPGTLSLSPGERATLSCRASQSVSS | | 226 |
| | SYLAWYQQKPGQAPRLLIYGASSRATGIP | | |
| | DRFSGSGSGTDFTLTISRLEPEDFAVYYCQ | | |
| | QYGSSPCTFGQGTRLEIKRTVAAPSVFIFPP | | |
| | SDEQLKSGTASVVCLLNNFYPREAKVQW | | |
| | KVDNALQSGNSQESVTEQDSKDSTYSLSS | | |
| | TLTLSKADYEKHKVYACEVTHQGLSSPVT | | |
| | KSFNRGEC | | |
| Heavy chain | EVQLVESGGGLVQPGGSLRLSCAASGFTF | From | 227 |
| variable | SNYWMNWVRQAPGKGLEWVAAINQDGS | WO2006013107 | |
| region | EKYYVGSVKGRFTISRDNAKNSLYLQMNS | SEQ ID NO: 8 | |
| | LRVEDTAVYYCVRDYYDILTDYYIHYWY | | |
| | FDLWGRGTLVTVSS | | |
| Light chain | EIVLTQSPGTLSLSPGERATLSCRASQSVSS | From | 228 |
| variable | SYLAWYQQKPGQAPRLLIYGASSRATGIP | WO2006013107 | |
| region | DRFSGSGSGTDFTLTISRLEPEDFAVYYCQ | SEQ ID NO: 10 | |
| | QYGSSPCTFGQGTRLEIKR | | |
| Heavy chain | NYWMN | From | 229 |
| CDR1 | | WO2006013107 | |
| | | SEQ ID NO: 1 | |
| Heavy chain | AINQDGSEKYYVGSVK | From | 230 |
| CDR2 | | WO2006013107 | |
| | | SEQ ID NO: 2 | |
| Heavy chain | DYYDILTDYYIHYWYFDL | From | 231 |
| CDR3 | | WO2006013107 | |
| | | SEQ ID NO: 3 | |
| Light chain | RASQSVSSSYLA | From | 232 |
| CDR1 | | WO2006013107 | |
| | | SEQ ID NO: 4 | |
| Light chain | GASSRAT | From | 233 |
| CDR2 | | WO2006013107 | |
| | | SEQ ID NO: 5 | |
| Light chain | QQYGSSPCT | From | 234 |
| CDR3 | | WO2006013107 | |
| | | SEQ ID NO: 6 | |

In one embodiment, polynucleotides encoding Secukinumab, fragments or variants thereof may be used to prevent, treat or manage an autoimmune disease, such as, but not limited to, psoriasis, including plaque psoriasis, guttate psoriasis, inverse psoriasis, pustular psoriasis, erythrodermic psoriasis, and psoriatic arthritis.

In one embodiment, the polynucleotides described herein may encode Secukinumab, a fragment or variant thereof and the polynucleotides may be used to treat and/or prevent psoriasis. In one embodiment, polynucleotides encoding Secukinumab, fragments or variants thereof may be used alone in combination with one or more additional treatment regimen as part of a maintenance regimen and/or at start of relapse for an autoimmune disease, such as, but not limited to, psoriasis.

In one embodiment, the polynucleotides described herein may encode Secukinumab, a fragment or variant thereof and the polynucleotides may be used to treat and/or prevent Rheumatoid arthritis. In one embodiment, polynucleotides encoding Secukinumab, fragments or variants thereof may be used to prevent, treat or manage an autoimmune disease, such as rheumatoid arthritis (RA),

In one embodiment, the polynucleotides described herein may encode Secukinumab, a fragment or variant thereof and the polynucleotides may be used to treat and/or prevent Psioratic Arthritis.

In one embodiment, the polynucleotides described herein may encode Secukinumab, a fragment or variant thereof and the polynucleotides may be used to treat and/or prevent Axial spondyloarthritis.

In one embodiment, polynucleotides encoding Secukinumab, fragments or variants thereof may be used to prevent, treat or manage an autoimmune disorder associated with elevated IL-17 levels.

### Ixekizumab Parent Molecule or Antibody

In one embodiment, the polynucleotides of the present invention may encode Ixekizumab, fragments or variants thereof.

Ixekizumab, also known as LY2439821, is a humanized IgG4 monoclonal antibody that binds to and neutralizes IL-17A developed by Eli Lilly and Co. IL-17A is a key pro-inflammatory cytokine produced by a subset of T helper cells called Th17 cells. Th17 cells are considered the main effectors of autoimmunity. Ixekizumab binds to IL-17A, thereby preventing it from interacting with its receptor, resulting in neutralization of the cytokine's activity.

Certain sequences encoding the polynucleotide fragments, domains or heavy or light chains for Ixekizumab are given in Table 48. The table is not an exhaustive list and any fragment or portion of the sequence which may be encoded in the polynucleotides of the invention.

TABLE 48
Table of Ixekizumab Sequences

| | | | |
|---|---|---|---|
| | | | SEQ |
| Descrip- | | | ID |
| tion | Sequence | Source | NO. |

| | | |
|---|---|---|
| Heavy | QVQLVQSGAEVKKPGSSVKVSCKASGYSF | 235 |
| chain | TDYHIHWVRQAPGQGLEWMGVINPMYG | |
| | TTDYNQRFKGRVTITADESTSTAYMELSS | |
| | LRSEDTAVYYCARYDYFTGTGVYWGQGT | |
| | LVTVSSASTKGPSVFPLAPCSRSTSESTAA | |
| | LGCLVKDYFPEPVTVSWNSGALTSGVHTF | |
| | PAVLQSSGLYSLSSVVTVPSSSLGTKTYTC | |
| | NVDHKPSNTKVDKRVESKYGPPCPPCPAP | |
| | EFLGGPSVFLFPPKPKDTLMISRTPEVTCV | |
| | VVDVSQEDPEVQFNWYVDGVEVHNAKT | |
| | KPREEQFNSTYRVVSVLTVLHQDWLNGK | |
| | EYKCKVSNKGLPSSIEKTISKAKGQPREPQ | |
| | VYTLPPSQEEMTKNQVSLTCLVKGFYPSDI | |
| | AVEWESNGQPENNYKTTPPVLDSDGSFFL | |
| | YSRLTVDKSRWQEGNVFSCSVMHEALHN | |
| | HYTQKSLSLSLG | |
| Light | DIVMTQTPLSLSVTPGQPASISCRSSRSLVH | 236 |
| chain | SRGNTYLHWYLQKPGQSPQLLIYKVSNRF | |
| | IGVPDRFSGSGSGTDFTLKISRVEAEDVGV | |
| | YYCSQSTHLPFTFGQGTKLEIKRTVAAPSV | |
| | FIFPPSDEQLKSGTASVVCLLNNFYPREAK | |
| | VQWKVDNALQSGNSQESVTEQDSKDSTY | |
| | SLSSTLTLSKADYEKHKVYACEVTHQGLS | |
| | SPVTKSFNRGEC | |

In one embodiment, polynucleotides encoding Ixekizumab, fragments or variants thereof may be used to prevent, treat or manage an autoimmune disorder associated with elevated IL-17 levels. As a non-limiting example, the polynucleotides encoding Ixekizumab may be used to prevent, treat or manage an autoimmune disorder as described for example in International Patent Publication WO2006013107, the contents of which is herein incorporated by reference in its entirety.

In one embodiment, polynucleotides encoding Ixekizumab, fragments or variants thereof may be used to prevent, treat or manage (for example as part of a maintenance regime) an autoimmune disease, such as psoriasis, including plaque psoriasis, pustular psoriasis, erythrodermic psoriasis, and psoriatic arthritis.

In one embodiment, polynucleotides encoding Ixekizumab, fragments or variants thereof may be used alone in combination with one or more additional treatment regimens as part of a maintenance regimen and/or at start of relapse for psoriasis.

In one embodiment, polynucleotides encoding Ixekizumab, fragments or variants thereof may be used in a method of treatment wherein the subject achieves a 20%, 50% or 70% improvement in the American College of Rheumatology core set disease index (ACR20, 50 or 70), for example after 12 weeks of treatment.

### Brodalumab Parent Molecule or Antibody

In one embodiment, the polynucleotides of the present invention may encode Brodalumab, fragments or variants thereof.

Brodalumab, also known as AMG827 and KHK4827, is a human monoclonal IgG2 antibody that selectively binds to and blocks signaling via the interleukin-17 (IL-17) cell surface receptor A (IL-17RA) currently in development by Amgen in collaboration with AstraZeneca through its MedImmune biologics unit. The IL-17 pathway plays an important role in inducing and promoting inflammatory disease processes. Brodalumab is a highly selective is a human monoclonal IgG2 antibody that binds to and blocks signaling via the IL-17 receptor.

Sequences of Brodalumab are described in International Patent Publication WO2006013107, the contents of which is incorporated herein by reference in its entirety. In one embodiment, the polynucleotides described herein may encode any of the Brodalumab or anti-IL-17 antibody sequences, variants or fragments thereof described in International Patent Publication WO2006013107, the contents of which is incorporated herein by reference in its entirety.

Certain sequences encoding the polynucleotide fragments, domains or heavy or light chains for Brodalumab are given in Table 49. The table is not an exhaustive list and any fragment or portion of the sequence which may be encoded in the polynucleotides of the invention.

TABLE 49
Table of Brodalumab Sequences

| | | | SEQ |
|---|---|---|---|
| | | | ID |
| Description | Sequence | Source | NO. |
| Heavy chain | MEWTWRVLFLVAAATGAHSQVQLVQSG | From | 237 |
| | AEVKKPGASVKVSCKASGYTFTRYGISWV | WO2006013107 | |
| | RQAPGQGLEWMGWISTYSGNTNYAQKLQ | SEQ ID NO: 427 | |
| | GRVTMTTDTSTSTAYMELRSLRSDDTAVY | | |
| | YCARRQLYFDYWGQGTLVTVSSASTKGP | | |
| | SVFPLAPCSRSTSESTAALGCLVKDYFPEP | | |
| | VTVSWNSGALTSGVHTFPAVLQSSGLYSL | | |
| | SSVVTVPSSNFGTQTYTCNVDHKPSNTKV | | |
| | DKTVERKCCVECPPCPAPPVAGPSVFLFPP | | |
| | KPKDTLMISRTPEVTCVVVDVSHEDPEVQ | | |
| | FNWYVDGVEVHNAKTKPREEQFNSTFRV | | |
| | VSVLTVVHQDWLNGKEYKCKVSNKGLPA | | |
| | PIEKTISKTKGQPREPQVYTLPPSREEMTK | | |
| | NQVSLTCLVKGFYPSDIAVEWESNGQPEN | | |
| | NYKTTPPMLDSDGSFFLYSKLTVDKSRWQ | | |
| | QGNVFSCSVMHEALHNHYTQKSLSLSPGK | | |
| Light chain | MEAPAQLLFLLLLWLPDTTGEIVMTQSPA | From | 238 |
| | TLSVSPGERATLSCRASQSVSSNLAWFQQ | WO2006013107 | |
| | KPGQAPRPLIYDASTRATGVPARFSGSGSG | SEQ ID NO: 429 | |
| | TDFTLTISSLQSEDFAVYYCQQYDNWPLT | | |
| | FGGGTKVEIKRTVAAPSVFIFPPSDEQLKS | | |
| | GTASVVCLLNNFYPREAKVQWKVDNALQ | | |
| | SGNSQESVTEQDSKDSTYSLSSTLTLSKAD | | |
| | YEKHKVYACEVTHQGLSSPVTKSFNRGEC | | |
| Heavy chain | RYGIS | From | 239 |
| CDR1 | | WO2006013107 | |
| | | SEQ ID NO: 146 | |
| Heavy chain | WISTYSGNTNYAQKLQG | From | 240 |
| CDR2 | | WO2006013107 | |
| | | SEQ ID NO: 147 | |
| Heavy chain | RQLYFDY | From | 241 |
| CDR3 | | WO2006013107 | |
| | | SEQ ID NO: 148 | |
| Light chain | RASQSVSSNLA | From | 242 |
| CDR1 | | WO2006013107 | |
| | | SEQ ID NO: 224 | |
| Light chain | DASTRAT | From | 243 |
| CDR2 | | WO2006013107 | |
| | | SEQ ID NO: 225 | |
| Light chain | QQYDNWPLT | From | 244 |
| CDR3 | | WO2006013107 | |
| | | SEQ ID NO: 226 | |

In one embodiment, polynucleotides encoding Brodalumab, fragments or variants thereof may be used to prevent, treat or manage an autoimmune disorder associated with elevated IL-17 levels. Non-limiting examples of methods for preventing, treating or managing an autoimmune disorder associated with elevated IL-17 levels is described in International Patent Publication WO2006013107, the content of which is herein incorporated by reference in its entirety, which may be used with the polynucleotides described herein encoding Brodalumab.

In one embodiment, polynucleotides encoding Brodalumab, fragments or variants thereof may be used to prevent, treat or manage (for example as part of a maintenance regime) an autoimmune disease, such as psoriasis, including plaque psoriasis, guttate psoriasis, inverse psoriasis, pustular psoriasis, erythrodermic psoriasis, and psoriatic arthritis.

In one embodiment, polynucleotides encoding Brodalumab, fragments or variants thereof may be used alone in combination with one or more additional treatment regimens as part of a maintenance regimen and/or at start of relapse for psoriasis.

In one embodiment, polynucleotides encoding Brodalumab, fragments or variants thereof may be used to prevent, treat or manage an autoimmune disease, such as such as psoriatic arthritis (PsA).

In one embodiment, polynucleotides encoding Brodalumab, fragments or variants thereof may be used alone in combination with one or more additional treatment regimens as part of a maintenance regimen and/or at start of relapse for an autoimmune disease, such as, but not limited to, PsA.

### Tildrakizumab Parent Molecule or Antibody

In one embodiment, the polynucleotides of the present invention may encode Tildrakizumab, fragments or variants thereof.

Tildrakizumab, also known as MK-3222 and SCH-900222, is a humanized IgG1 monoclonal antibody that binds Interleukin-23 (IL-23) p19 subunit and neutralizes IL-23 which was originally developed by Schering Plough and is currently in development by Merck. Interleukin-23 (IL-23) is a key pro-inflammatory cytokine produced by activated myeloid cells, as well as epithelial and endothelial cells and is composed of two subunits, p19 and p40. P40 is shared with IL-12, while p19 mediates the distinctive biological action of IL-23.

Sequences of Tildrakizumab are described in U.S. Pat. No. 8,263,748, the contents of which is incorporated herein by reference in its entirety. In one embodiment, the polynucleotides described herein may encode any of the Tildrakizumab or anti-IL-23 antibody sequences, variants or fragments thereof described in U.S. Pat. No. 8,263,748, the contents of which is incorporated herein by reference in its entirety.

Certain sequences encoding the polynucleotide fragments, domains or heavy or light chains for Tildrakizumab are given in Table 50. The table is not an exhaustive list and any fragment or portion of the sequence which may be encoded in the polynucleotides of the invention.

TABLE 50
Table of Tildrakizumab Sequences

| | | | SEQ |
|---|---|---|---|
| | | | ID |
| Description | Sequence | Source | NO. |
| Heavy chain | QVQLVQSGAEVKKPGASVKVSCKASGYI | From U.S. Pat. | 245 |
| | FITYWMTWVRQAPGQGLEWMGQIFPASG | No. 8,263,748 | |
| | SADYNEKFEGRVTMTTDTSTSTAYMELRS | SEQ ID NO: 7 | |
| | LRSDDTAVYYCARGGGGFAYWGQGTLV | | |
| | TVSSASTKGPSVFPLAPSSKSTSGGTAALG | | |
| | CLVKDYFPEPVTVSWNSGALTSGVHTFPA | | |
| | VLQSSGLYSLSSVVTVPSSSLGTQTYICNV | | |
| | NHKPSNTKVDKKVEPKSCDKTHTCPPCPA | | |
| | PELLGGPSVFLFPPKPKDTLMISRTPEVTC | | |
| | VVVDVSHEDPEVKFNWYVDGVEVHNAK | | |
| | TKPREEQYNSTYRVVSVLTVLHQDWLNG | | |
| | KEYKCKVSNKALPAPIEKTISKAKGQPREP | | |
| | QVYTLPPSRDELTKNQVSLTCLVKGFYPS | | |
| | DIAVEWESNGQPENNYKTTPPVLDSDGSF | | |
| | FLYSKLTVDKSRWQQGNVFSCSVMHEAL | | |
| | HNHYTQKSLSLSPGK | | |
| Light chain | DIQMTQSPSSLSASVGDRVTITCRTSENIY | From U.S. Pat. | 246 |
| | SYLAWYQQKPGKAPKLLIYNAKTLAEGV | No. 8,263,748 | |
| | PSRFSGSGSGTDFTLTISSLQPEDFATYYCQ | SEQ ID NO: 17 | |
| | HHYGIPFTFGQGTKVEIKRTVAAPSVFIFPP | | |
| | SDEQLKSGTASVVCLLNNFYPREAKVQW | | |
| | KVDNALQSGNSQESVTEQDSKDSTYSLSS | | |
| | TLTLSKADYEKHKVYACEVTHQGLSSPVT | | |
| | KSFNRGEC | | |
| Light chain | RTSENIYSYLA | From U.S. Pat. | 247 |
| CDR1 | | No. 8,263,748 | |
| | | SEQ ID NO: 36 | |
| Light chain | NAKTLAE | From U.S. Pat. | 248 |
| CDR2 | | No. 8,263,748 | |
| | | SEQ ID NO: 41 | |
| Light chain | QHHYGIPFT | From U.S. Pat. | 249 |
| CDR3 | | No. 8,263,748 | |
| | | SEQ ID NO: 46 | |
| Heavy chain | GYIFITYWMT | From U.S. Pat. | 250 |
| CDR1 | | No. 8,263,748 | |
| | | SEQ ID NO: 19 | |
| Heavy chain | QIFPASGSADYNEMFE | From U.S. Pat. | 251 |
| CDR2 | | No. 8,263,748 | |
| | | SEQ ID NO: 24 | |
| Heavy chain | QIFPASGSADYNEKFE | From U.S. Pat. | 252 |
| CDR2 | | No. 8,263,748 | |
| | | SEQ ID NO: 25 | |
| Heavy chain | QIFPASGSADYAQKLQ | From U.S. Pat. | 253 |
| CDR2 | | No. 8,263,748 | |
| | | SEQ ID NO: 26 | |
| Heavy chain | GGGGFAY | From U.S. Pat. | 254 |
| CDR3 | | No. 8,263,748 | |
| | | SEQ ID NO: 31 | |

In one embodiment, polynucleotides encoding Tildrakizumab, fragments or variants thereof may be used to prevent, treat or manage an autoimmune disease, such as, but not limited to, psoriasis, including plaque psoriasis, guttate psoriasis, inverse psoriasis, pustular psoriasis, erythrodermic psoriasis, and psoriatic arthritis.

In one embodiment, polynucleotides encoding Tildrakizumab, fragments or variants thereof may be used to treat and/or prevent psoriasis.

In one embodiment, polynucleotides encoding Tildrakizumab, fragments or variants thereof may be used to treat and/or prevent plaque psoriasis.

In one embodiment, polynucleotides encoding Tildrakizumab, fragments or variants thereof may be used to prevent, treat or manage an autoimmune disorder associated with elevated IL-23 levels by eliciting a IL-23 blockade, such as, but not limited to, inflammatory bowel disease, Crohn's disease, ulcerative Colitis, rheumatoid arthritis, psoriatic arthritis, psoriasis, ankylosing spondylitis, and atopic dermatitis.

In one embodiment, polynucleotides encoding Tildrakizumab, fragments or variants thereof may be used alone in combination with one or more additional treatment regimen as part of a maintenance regimen and/or at start of relapse for psoriasis.

In one embodiment, polynucleotides encoding Tildrakizumab, fragments or variants thereof may be used in a method of treatment for psoriasis, wherein the subject achieves a 20%, 50% or 77% improvement in the Psoriasis Area and Severity Index (PASI20, PASI50 or PASI75), for example after 12 weeks of treatment.

### Tabalumab Parent Molecule or Antibody

In one embodiment, the polynucleotides of the present invention may encode Tabalumab, fragments or variants thereof.

Tabalumab, also known as LY2127399, is a human monoclonal antibody which binds to and inhibits the activity of soluble and cell surface-bound B cell activating factor BAFF (also called TNFSF13b, BLyS, TALL-1, THANK, neutrokine-α, and zTNF-BLyS), being developed by Eli Lilly and Co. BAFF belongs to the tumor necrosis factor (TNF) family (BAFF), plays an important role in B cell development, and is implicated in autoimmune diseases, such as SLE, and B cell malignancies, including MM (Sun et al, 2008).

Sequences of Tabalumab are described in U.S. Pat. No. 7,317,089 and U.S. Pat. No. 8,173,124, the contents of each of which is incorporated herein by reference in its entirety. In one embodiment, the polynucleotides described herein may encode any of the Tabalumab or anti-BAFF antibody sequences, variants or fragments thereof described in U.S. Pat. No. 7,317,089 and U.S. Pat. No. 8,173,124, the contents of which is incorporated herein by reference in its entirety.

Certain sequences encoding the polynucleotide fragments, domains or heavy or light chains for Tabalumab are given in Table 51. The table is not an exhaustive list and any fragment or portion of the sequence which may be encoded in the polynucleotides of the invention.

TABLE 51
Table of Tabalumab Sequences

| | | | SEQ |
|---|---|---|---|
| | | | ID |
| Description | Sequence | Source | NO. |
| Heavy chain | QVQLQQWGAGLLKPSETLSLTCAVYGGS | From U.S. Pat. | 255 |
| | FSGYYWSWIRQPPGKGLEWIGEINHSGST | No. 7,317,089 | |
| | NYNPSLKSRVTISVDTSKNQFSLKLSSVTA | and | |
| | ADTAVYYCARGYYDILTGYYYYFDYWG | U.S. 8,173,124 | |
| | QGTLVTVSSASTKGPSVFPLAPCSRSTSES | SEQ ID NO: 17 | |
| | TAALGCLVKDYFPEPVTVSWNSGALTSGV | | |
| | HTFPAVLQSSGLYSLSSVVTVPSSSLGTKT | | |
| | YTCNVDHKPSNTKVDKRVESKYGPPCPPC | | |
| | PAPEFLGGPSVFLFPPKPKDTLMISRTPEVT | | |
| | CVVVDVSQEDPEVQFNWYVDGVEVHNA | | |
| | KTKPREEQFNSTYRVVSVLTVLHQDWLN | | |
| | GKEYKCKVSNKGLPSSIEKTISKAKGQPRE | | |
| | PQVYTLPPSQEEMTKNQVSLTCLVKGFYP | | |
| | SDIAVEWESNGQPENNYKTTPPVLDSDGS | | |
| | FFLYSRLTVDKSRWQEGNVFSCSVMHEAL | | |
| | HNHYTQKSLSLSLGK | | |
| Light chain | EIVLTQSPATLSLSPGERATLSCRASQSVS | From U.S. Pat. | 256 |
| | RYLAWYQQKPGQAPRLLIYDASNRATGIP | No. 7,317,089 | |
| | ARFSGSGSGTDSTLTISSLEPEDFAVYYCQ | and 8,173,124 | |
| | QRSNWPRTFGQGTKVEIKRTVAAPSVFIFP | SEQ ID NO: 19 | |
| | PSDEQLKSGTASVVCLLNNFYPREAKVQ | | |
| | WKVDNALQSGNSQESVTEQDSKDSTYSLS | | |
| | NTLTLSKADYEKHKVYACEVTHQGLSSPV | | |
| | TKSFNRGEC | | |

In one embodiment, polynucleotides encoding Tabalumab, fragments or variants thereof may be used to prevent, treat or manage an autoimmune disease by preventing B cell activation, proliferation, and survival.

In one embodiment, polynucleotides encoding Tabalumab, fragments or variants thereof may be used to prevent, treat or manage an autoimmune disease, such as, but not limited to, rheumatoid arthritis (RA). In one embodiment, a subject may have been previously treated with a TNF antagonist and/or was intolerant to an TNF antagonist. In another embodiment the subject was previously ineffectively treated for RA by administering DMARDS, such as methotrexate, leflunomide, sulfasalazine and/or hydroxychloroquine. In one embodiment, polynucleotides encoding Tabalumab, fragments or variants thereof may be used to prevent, treat or manage rheumatoid arthritis (RA) in combination with DMARDs, such as, but not limited to, methotrexate.

In one embodiment, polynucleotides encoding Tabalumab, fragments or variants thereof may be used alone in combination with one or more additional treatment regime as part of a maintenance regimen and/or at start of relapse for an autoimmune disease, such as RA.

In one embodiment, polynucleotides encoding Tabalumab, fragments or variants thereof may be used to prevent, treat or manage systemic lupus erythromatosis (SLE). In one embodiment, polynucleotides encoding Tabalumab, fragments or variants thereof may be used to prevent, treat or manage an autoimmune disease, such as SLE in combination at least one other antibody, such as, but not limited to belilumab, rituximab, epratuzumab, aontalizumab, sifalimumab, ASG-009, and/or the recombinant fusion protein atacicept. The combination antibody or recombinant fusion protein may be encoded by the polynucleotides described herein.

In one embodiment, polynucleotides encoding tabalumab, fragments or variants thereof may be used to prevent, treat or manage multiple myeloma (MM).

### Itolizumab Parent Molecule or Antibody

In one embodiment, the polynucleotides of the present invention may encode Itolizumab, fragments or variants thereof.

Itolizumab, also known as clone T1h, is a humanized IgG1 monoclonal antibody directed against CD6, a surface glycoprotein expressed on the majority of T cells and a subset of B cells developed by Biocon Limited. Itolizumab was originally derived from the mouse monoclonal sequence of IOR-T1. CD6 is involved in co-stimulation, adhesion and maturation of T cells. The CD6 protein is found on the outer membrane of T-lymphocytes as well as some other immune cells and is involved in co-stimulation, adhesion and maturation of T cells. The CD6 co-stimulatory pathway contributes to the Th1 activation and differentiation of human T cells, promoting a pro-inflammatory response (Nair et al, 2010). A dysregulated CD6 signaling process may lead to uncontrolled tissue inflammation and an autoimmune pathology.

Sequences of Itolizumab are described in International Patent Publication No. WO2009113083 and U.S. Pat. No. 6,572,857, the contents of each of which is incorporated herein by reference in its entirety. In one embodiment, the polynucleotides described herein may encode any of the Itolizumab antibody sequences, variants or fragments thereof described in International Patent Publication No. WO2009113083 and U.S. Pat. No. 6,572,857, the contents of which is incorporated herein by reference in its entirety.

Certain sequences encoding the polynucleotide fragments, domains or heavy or light chains for Itolizumab are given in Table 52. The table is not an exhaustive list and any fragment or portion of the sequence which may be encoded in the polynucleotides of the invention.

TABLE 52
Table of Itolizumab Sequences

| | | | SEQ |
|---|---|---|---|
| | | | ID |
| Description | Sequence | Source | NO. |
| Heavy chain | EVQLVESGGGLVKPGGSLKLSCAASGFKF | | 257 |
| | SRYAMSWVRQAPGKRLEWVATISSGGSYI | | |
| | YYPDSVKGRFTISRDNVKNTLYLQMSSLR | | |
| | SEDTAMYYCARRDYDLDYFDSWGQGTL | | |
| | VTVSSASTKGPSVFPLAPSSKSTSGGTAAL | | |
| | GCLVKDYFPEPVTVSWNSGALTSGVHTFP | | |
| | AVLQSSGLYSLSSVVTVPSSSLGTQTYICN | | |
| | VNHKPSNTKVDKKVEPKSCDKTHTCPPCP | | |
| | APELLGGPSVFLFPPKPKDTLMISRTPEVT | | |
| | CVVVDVSHEDPEVKFNWYVDGVEVHNA | | |
| | KTKPREEQYNSTYRVVSVLTVLHQDWLN | | |
| | GKEYKCKVSNKALPAPIEKTISKAKGQPR | | |
| | EPQVYTLPPSRDELTKNQVSLTCLVKGFY | | |
| | PSDIAVEWESNGQPENNYKTTPPVLDSDG | | |
| | SFFLYSKLTVDKSRWQQGNVFSCSVMHE | | |
| | ALHNHYTQKSLSLSPGK | | |
| Light chain | DIQMTQSPSSLSASVGDRVTITCKASRDIR | | 258 |
| | SYLTWYQQKPGKAPKTLIYYATSLADGVP | | |
| | SRFSGSGSGQDYSLTISSLESDDTATYYCL | | |
| | QHGESPFTLGSGTKLEIKRTVAAPSVFIFPP | | |
| | SDEQLKSGTASVVCLLNNFYPREAKVQW | | |
| | KVDNALQSGNSQESVTEQDSKDSTYSLSS | | |
| | TLTLSKADYEKHKVYACEVTHQGLSSPVT | | |
| | KSFNRGEC | | |
| Heavy chain | EVQLVESGGGLVKPGGSLKLSCAASGFKF | From | 259 |
| sequence | SRYAMSWVRQAPGKRLEWVATISSGGSYI | WO2009113083 | |
| | YYPDSVKGRFTISRDNVKNTLYLQMSSLR | SEQ ID NO: 1 | |
| | SEDTAMYYCARRDYDLDYFDSWGQGTL | | |
| | VTVSS | | |
| Light chain | DIQMTQSPSSLSASVGDRVTITCKASRDIR | From | 260 |
| sequence | SYLTWYQQKPGKAPKTLIYYATSLADGVP | WO2009113083 | |
| | SRFSGSGSGQDYSLTISSLESDDTATYYCL | SEQ ID NO: 2 | |
| | QHGESPFTLGSGTKLEIK | | |
| Heavy chain | EVQLVESGGGLVKPGGSLKLSCAASGFKF | From U.S. Pat. | 261 |
| sequence | SRYAMSWVRQAPGKRLEWVATISSGGSYI | No. 6,572,857 | |
| | YYPDSVKGRFTISRDNVKNTLYLQMSSLR | SEQ ID NO: 3 | |
| | SEDTAMYYCARRDYDLDYFDSWGQGTL | | |
| | VTVS | | |
| Light chain | DIQMTQSPSSLSASVGDRVTITCKASRDIR | From U.S. Pat. | 262 |
| sequence | SYLTWYQQKPGKAPKTLIYYATSLADGVP | No. 6,572,857 | |
| | SRFSGSGSGQDYSLTISSLESDDTATYYCL | SEQ ID NO: 4 | |
| | QHGESPFTFGSGTKLEIKRA | | |

In one embodiment, polynucleotides encoding Itolizumab, fragments or variants thereof may be used to prevent, treat or manage psoriasis.

In one embodiment, the polynucleotides described herein encoding Itolizumab, fragments or variants thereof may be used to prevent, treat and/or manage plaque psoriasis, guttate psoriasis, inverse psoriasis, pustular psoriasis, erythrodermic psoriasis, and psoriatic arthritis.

In one embodiment, polynucleotides encoding Itolizumab, fragments or variants thereof may be used alone in combination with one or more additional treatment regimen, as part of a maintenance regimen and/or at start of relapse for an autoimmune disease, such as, but not limited to, psoriasis.

### Ocrelizumab Parent Molecule or Antibody

In one embodiment, the polynucleotides of the present invention may encode Ocrelizumab, fragments or variants thereof.

Ocrelizumab, also known as hu2H7 v16, UNII-A10SJL62JY, rhuMAb 2H7 and OCRE, is a humanized recombinant type 1 monoclonal antibody that selectively targets CD20-positive B-cells in development by Genetech. Ocrelizumab immunosuppressive agent then interacts with the body's immune system to eliminate CD20-positive B-cells. Ocrelizumab contains human framework regions and the complementarity-determining regions (CDRs) of a murine antibody that binds to CD20 (Liu, A. Y. et al., J. Immunol., 1987, 10, 3521-3526; the contents of which is incorporated herein by reference in entirety).

Ocrelizumab is a humanized version of murine monoclonal antibody 2H7. The three complementary determining regions (CDR1, CDR 2 and CDR3) of humanized light chain have amino acid sequences are derived from the corresponding CDRs of the mouse immunoglobulin light chain variable regions. Similarly, the three complementary determining regions (CDR1, CDR 2 and CDR3) of humanized heavy chain have amino acid sequences from the corresponding CDRs of the mouse immunoglobulin heavy chain variable regions and a variable region framework from a human heavy chain variable framework sequence. The constant regions are substantially from a human immunoglobulin.

The Fc portion of Ocrelizumab was modified to reduce complement-dependent cytotoxicity, because complement activation may lead to some of the side effects associated with Rituximab, a currently marketed anti-CD20 monoclonal antibody. Ocrelizumab binds to a different, but overlapping, epitope of the extracellular domain of CD20 compared with Rituximab (Genovese M. C. et al. Arthritis & Rheumatism, 2008, 58(9), 2652-2661; incorporated herein by reference in entirety).

Ocrelizumab is described in US Patent Publication Nos. 20040202658 and 20120225070 and U.S. Pat. No. 7,708,994, the contents of each of which is herein incorporated by reference in its entirety. In one embodiment, the polynucleotides described herein encode an Ocrelizumab sequence described in The complementarity determining regions (CDRs) of Ocrelizumab are identified in US Patent Publication Nos. 20040202658 and 20120225070 and U.S. Pat. No. 7,708,994, the contents of each of which is herein incorporated by reference in its entirety. In one embodiment, the polynucleotides described herein encode at least one CDR for Ocrelizumab described in US Patent Publication No. 20120225070, the contents of which is incorporated herein by reference in entirety.

Certain sequences encoding the polynucleotide fragments, domains or heavy or light chains for Ocrelizumab are given in Table 53. The table is not an exhaustive list and any fragment or portion of the sequence which may be encoded in the polynucleotides of the invention.

TABLE 53
Table of Ocrelizumab Sequences

| | | | SEQ |
|---|---|---|---|
| | | | ID |
| Description | Sequence | Source | NO. |
| Variable | DIQMTQSPSSLSASVGDRVTITCRASSSVS | From U.S. | 263 |
| light-chain | YMHWYQQKPGKAPKPLIYAPSNLASGV | 20040202658 | |
| domain of | PSRFSGSGSGTDFTLTISSLQPEDFATYYC | SEQ ID NO: 1 | |
| hu2H7 v16 | QQWSFNPPTFGQGTKVEIKR | (CDRs in bold, as | |
| | | identified in | |
| | | US20120225070) | |
| Variable | EVQLVESGGGLVQPGGSLRLSCAASGYT | From U.S. | 264 |
| heavy-chain | FTSYNMHWVRQAPGKGLEWVGAIYPG | 20040202658 | |
| domain of | NGDTSYNQKFKGRFTISVDKSKNTLYLQ | SEQ ID NO: 2 | |
| hu2H7 v16 | MNSLRAEDTAVYYCARVVYYSNSYWYF | (CDRs in bold as | |
| | DVWGQGTLVTVSS | identified in | |
| | | US20120225070) | |
| Humanized | DIQMTQSPSSLSASVGDRVTITCRASSSVS | From U.S. | 265 |
| hu2H7 v16 | YMHWYQQKPGKAPKPLIYAPSNLASGVP | 20040202658 | |
| light chain | SRFSGSGSGTDFTLTISSLQPEDFATYYCQ | SEQ ID NO: 3 | |
| amino acid | QWSFNPPTFGQGTKVEIKRTVAAPSVFIF | | |
| sequence | PPSDEQLKSGTASVVCLLNNFYPREAKV | | |
| | QWKVDNALQSGNSQESVTEQDSKDSTYS | | |
| | LSSTLTLSKADYEKHKVYACEVTHQGLS | | |
| | SPVTKSFNRGEC | | |
| Humanized | EVQLVESGGGLVQPGGSLRLSCAASGYT | From U.S. | 266 |
| 2H7 v16 | FTSYNMHWVRQAPGKGLEWVGAIYPGN | 20040202658 | |
| heavy chain | GDTSYNQKFKGRFTISVDKSKNTLYLQM | SEQ ID NO: 4 | |
| amino acid | NSLRAEDTAVYYCARVVYYSNSYWYFD | | |
| sequence | VWGQGTLVTVSSASTKGPSVFPLAPSSKS | | |
| | TSGGTAALGCLVKDYFPEPVTVSWNSGA | | |
| | LTSGVHTFPAVLQSSGLYSLSSVVTVPSSS | | |
| | LGTQTYICNVNHKPSNTKVDKKVEPKSC | | |
| | DKTHTCPPCPAPELLGGPSVFLFPPKPKD | | |
| | TLMISRTPEVTCVVVDVSHEDPEVKFNW | | |
| | YVDGVEVHNAKTKPREEQYNSTYRVVS | | |
| | VLTVLHQDWLNGKEYKCKVSNKALPAPI | | |
| | EKTISKAKGQPREPQVYTLPPSREEMTKN | | |
| | QVSLTCLVKGFYPSDIAVEWESNGQPEN | | |
| | NYKTTPPVLDSDGSFFLYSKLTVDKSRW | | |
| | QQGNVFSCSVMHEALHNHYTQKSLSLSP | | |
| | GK | | |

In one embodiment, polynucleotides encoding Ocrelizumab, fragments or variants thereof may be used to prevent, treat or manage multiple sclerosis (MS). As a non-limiting example, the polynucleotides may be used in combination with interferon beta 1a (also known as REBIFI®). In some embodiments, the polynucleotides encoding Ocrelizumab may be used to treat multiple sclerosis, such as relapsing-remitting multiple sclerosis, secondary progressive multiple sclerosis, primary progressive multiple sclerosis, or clinically isolated syndrome. MS is a serious and disabling inflammatory and autoimmune disease of young adults, with a peak age of onset in the third decade of life. Most individuals present with the relapsing-remitting form of the disease and experience recurrent attacks, which, over time, result in accumulating permanent physical disability and cognitive decline. While the cause is not clear, the underlying mechanism is thought to be either destruction by the immune system or failure of the myelin-producing cells. Proposed causes for this include genetics and environmental factors such as infections. MS is usually diagnosed based on the presenting signs and symptoms and the results of supporting medical tests. Almost 70% of patients will develop secondary progressive MS. Current treatments are minimally effective for secondary progressive MS.

In one embodiment, the polynucleotides encoding Ocrelizumab may be used to reduce the number of flare ups in a disease or disorder such as, but not limited to, MS.

In some embodiments, the polynucleotides encoding Ocrelizumab may be used to block the triggering of complement-dependent cell lysis (CDCL) and antibody-dependent cell-mediated cytotoxicity (ADCC) of B-cells overexpressing CD20.

In some embodiments, the polynucleotides encoding ocrelizumab may be used to treat CD20-positive malignancies and autoimmune diseases such as arthritis (rheumatoid arthritis, juvenile rheumatoid arthritis, osteoarthritis, psoriatic arthritis), psoriasis, dermatitis including atopic dermatitis; chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), respiratory distress syndrome, adult respiratory distress syndrome (ARDS), meningitis, allergic rhinitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), lupus (including nephritis, non-renal, discoid, alopecia), juvenile onset diabetes, multiple sclerosis, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues), graft versus host disease (GVHD), pemphigoid bullous, pemphigus (all including vulgaris, foliaceus), autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant) vs NSIP, Guillain-Barre' syndrome, large vessel vasculitis (including polymyalgia rheumatica and giant cell (Takayasu's) arteritis), medium vessel vasculitis (including Kawasaki's disease and polyarteritis nodosa), ankylosing spondylitis, Berger's Disease (IgA nephropathy), Rapidly Progressive Glomerulonephritis, Primary biliary cirrhosis, celiac sprue (gluten enteropathy), cryoglobulinemia, ALS, and coronary artery disease (see e.g. U.S. Pat. Nos. 8,562,992; 8,545,843 and 7,799,900; the contents of each of which are incorporated by reference in their entirety).

### Epratuzumab Parent Molecule or Antibody

In one embodiment, the polynucleotides of the present invention may encode Epratuzumab, fragments or variants thereof.

Epratuzumab, also known as LL2, EPB2 and LYMPHOCIDE™, is a humanized IgG1 monoclonal antibody targeting CD22, a cell surface glycoprotein present on mature B-cells and on many different types of malignant B-cells developed by Immunomedics and licensed to UCB S.A. Epratuzumab is classified as an immunomodulatory agent.

Epratuzumab binds with high specificity to normal B-cells and B-cell tumors at the third Ig-like domain of CD22. After binding to CD22, epratuzumab's predominant antitumor activity appears to be mediated through antibody-dependent cellular cytotoxicity (ADCC). Mechanisms of action appear to differ from those of rituximab, specifically by the ability of epratuzumab to induce CD22 phosphorylation, modulate the B cell receptor, as well as to mediate a moderate degree of ADCC, without induction of apoptosis or complement-mediated cell lysis (Leonard, J. P. et al. Oncogene, 2007, 26, 3704-3713; incorporated herein by reference in entirety).

Epratuzumab is a humanized monoclonal antibody derived from the murine IgG2a monoclonal antibody, LL2 (EPB-2). It has a molecular weight of 150 KD. The three complementarity determining regions (CDR1, CDR 2 and CDR3) of the humanized light chain have amino acid sequences are derived from the corresponding CDRs of the mouse immunoglobulin light chain variable regions. Similarly, the three complementary determining regions (CDR1, CDR 2 and CDR3) of humanized heavy chain have amino acid sequences from the corresponding CDRs of the mouse immunoglobulin heavy chain variable regions. The constant regions are substantially from a human immunoglobulin. U.S. Pat. No. 5,789,554 (the contents of which is incorporated herein by reference in entirety), assigned to Immunomedics is the first US patent describing the CDRs in heavy and light chains of a humanized anti-CD22 antibody based on the complementarity determining regions of murine anti-CD22 antibody LL2. In one embodiment, the polynucleotides described herein encode Epratuzumab sequence, a fragment or variant thereof described in U.S. Pat. No. 5,789,554 and International Patent Publication No. WO2011032633, the contents of which is herein incorporated by reference in its entirety.

Certain sequences encoding the polynucleotide fragments, domains or heavy or light chains for Epratuzumab are given in Table 54. The table is not an exhaustive list and any fragment or portion of the sequence which may be encoded in the polynucleotides of the invention.

TABLE 54
Table of Epratuzumab Sequences

| | | | SEQ |
|---|---|---|---|
| | | | ID |
| Description | Sequence | Source | NO. |
| Light chain | DIQLTQSPSSLSASVGDRVTMSCKSSQSV | From PCT | 267 |
| of | LYSANHKNYLAWYQQKPGKAPKLLIYW | Publication No. | |
| epratuzumab | ASTRESGVPSRFSGSGSGTDFTFTISSLQP | WO/2011/032633 | |
| | EDIATYYCHQYLSSWTFGGGTKVQIKRT | -SEQ ID NO: 1 | |
| | VAAPSVFIFPPSDEQLKSGTASVVCLLNN | (CDRs in bold as | |
| | FYPREAKVQWKVDNALQSGNSQESVTE | identified in U.S. | |
| | QDSKDSTYSLSSTLTLSKADYEKHKVYA | Pat. 5,789,554 | |
| | CEVTHQGLSSPVTKSFNRGEC (219) | | |
| Heavy chain | QVQLVQSGAEVKKPGSSVKVSCKASGYT | From PCT | 268 |
| of | FTSYWLHWVRQAPGQGLEWIGYINPRN | Publication No. | |
| epratuzumab | DYTEYNQNFKDKATITADESTNTAYMEL | WO/2011/032633 | |
| | SSLRSEDTAFYFCARRDITTFYWGQGTT | -SEQ ID NO: 2 | |
| | VTVSSASTKGPSVFPLAPSSKSTSGGTAA | (CDRs in bold as | |
| | LGCLVKDYFPEPVTVSWNSGALTSGVHT | identified in U.S. | |
| | FPAVLQSSGLYSLSSVVTVPSSSLGTQTYI | Pat. 5,789,554 | |
| | CNVNHKPSNTKVDKRVEPKSCDKTHTCP | | |
| | PCPAPELLGGPSVFLFPPKPKDTLMISRTP | | |
| | EVTCVVVDVSHEDPEVKFNWYVDGVEV | | |
| | HNAKTKPREEQYNSTYRVVSVLTVLHQD | | |
| | WLNGKEYKCKVSNKALPAPIEKTISKAK | | |
| | GQPREPQVYTLPPSREEMTKNQVSLTCL | | |
| | VKGFYPSDIAVEWESNGQPENNYKTTPP | | |
| | VLDSDGSFFLYSKLTVDKSRWQQGNVFS | | |
| | CSVMHEALHNHYTQKSLSLSPGK (446) | | |

In one embodiment, polynucleotides encoding Epratuzumab, fragments or variants thereof may be used to prevent, treat or manage systemic lupus erythematosus (SLE), leukemia (lymphoid), and lymphoma (non-Hodgkin's Lymphoma).

In one embodiment, polynucleotides encoding Epratuzumab, fragments or variants thereof may be used to prevent, treat or manage systemic lupus erythematosus (SLE).

In one embodiment, polynucleotides encoding Epratuzumab, fragments or variants thereof may be used to prevent, treat or manage autoimmune diseases related to aberrant B cell function, such as, but not limited to, primary Sjorgen's syndrome (Steinfeld, S. D. et al. Expert Opin. Biol. Ther. 2006, 6(9), 943-949; incorporated herein by reference in entirety) and other B cell malignancies (Leonard, J. P. et al. Oncogene, 2007, 26, 3704-3713; incorporated herein by reference in entirety).

In some embodiments, the polynucleotides encoding Epratuzumab may be used to treat autoimmune diseases such as acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis ubiterans, Sjogren's syndrome, primary biliary cinhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis (see for example; U.S. Pat. No. 7,641,901, the contents of which is incorporated herein by reference in entirety).

In some embodiments, the polynucleotides encoding Epratuzumab may be used to treat cancers or malignancies, such as B-cell lymphomas and leukemia (see, e.g. U.S. Pat. Nos. 5,789,554; 6,187,287 and 8,105,596; the contents of each of which are incorporated by reference in their entirety.) For example, the polynucleotides encoding epratuzumab may be used in combination with other agents such as a steroid (e.g. glucocorticoid), an anti-inflammatory compound, an immunosuppressive compound, and an antioxidant, or a chemotherapeutic agent.

### Gantenerumab Parent Molecule or Antibody

In one embodiment, the polynucleotides of the present invention may encode Gantenerumab, fragments or variants thereof.

Gantenerumab, also known as R04909832 and RG1450, is a monoclonal immunoglobulin IgG1 antibody designed to bind with subnanomolar affinity to a conformational epitope on amyloid-β fibrils in development by Chugai Pharmaceuticals. Gantenerumab selected from a synthetic human combinatorial antibody library (HuCAL®; MorphoSys, Martinsried/Planegg, Germany) based on phage display technology and was optimized by in vitro affinity maturation.

Gantenerumab is unique amongst the anti-amyloid-β therapeutic antibodies in development in that it binds to both the N-terminus and mid-section of the 42 amino acid amyloid-β peptide. It has been shown to break down amyloid plaque both in vitro and in vivo.

Gantenerumab passes the blood-brain barrier and has a high binding affinity to cerebral amyloid plaques. The proposed binding mode of gantenerumab to fibrillar amyloid-β involves both N-terminal and spatially adjacent central amyloid-β sequences. According to this model, the flexible N-terminals of amyloid-β are the initial contact points of gantenerumab binding, followed by interaction with adjacent central amyloid-β, part of which confers increased binding stability. The specificity of gantenerumab predicts strong binding to native amyloid-β plaques (Novakovic D., et al., Drug Design Dev. Ther. 2013, 7, 1359-1364, incorporated herein by reference in entirety).

Certain sequences encoding the polynucleotide fragments, domains or heavy or light chains for Gantenerumab are given in Table 55. The table is not an exhaustive list and any fragment or portion of the sequence which may be encoded in the polynucleotides of the invention.

TABLE 55
Table of Gantenerumab Sequences

| | | | SEQ |
|---|---|---|---|
| | | | ID |
| Description | Sequence | Source | NO. |
| Heavy chain | QVELVESGGGLVQPGGSLRLSCAASGF | Immunogenetics | 269 |
| of | TFSSYAMSWVRQAPGKGLEWVSAINA | Information | |
| gantenerumab | SGTRTYYADSVKGRFTISRDNSKNTLY | System; CHAIN | |
| | LQMNSLRAEDTAVYYCARGKGNTHK | ID 8894_H. | |
| | PYGYVRYFDVWGQGTLVTVSSASTKG | CDRs are shown | |
| | PSVFPLAPSSKSTSGGTAALGCLVKDYF | in bold. | |
| | PEPVTVSWNSGALTSGVHTFPAVLQSS | (www.imgt.org/ | |
| | GLYSLSSVVTVPSSSLGTQTYICNVNHK | mAb-DB/query | |
| | PSNTKVDKKVEPKSCDKTHTCPPCPAPE | Query: | |
| | LLGGPSVFLFPPKPKDTLMISRTPEVTCV | gantanerumab) | |
| | VVDVSHEDPEVKFNWYVDGVEVHNAK | | |
| | TKPREEQYNSTYRVVSVLTVLHQDWLN | | |
| | GKEYKCKVSNKALPAPIEKTISKAKGQP | | |
| | REPQVYTLPPSRDELTKNQVSLTCLVKG | | |
| | FYPSDIAVEWESNGQPENNYKTTPPVLD | | |
| | SDGSFFLYSKLTVDKSRWQQGNVFSCS | | |
| | VMHEALHNHYTQKSLSLSPGK (456) | | |
| Light chain of | DIVLTQSPATLSLSPGERATLSCRASQSV | Immunogenetics | 270 |
| gantenerumab | SSSYLAWYQQKPGQAPRLLIYGASSRA | Information | |
| | TGVPARFSGSGSGTDFTLTISSLEPEDFA | System; CHAIN | |
| | TYYCLQIYNMPITFGQGTKVEIKRTVA | ID 8894_L. | |
| | APSVFIFPPSDEQLKSGTASVVCLLNNF | CDRs are shown | |
| | YPREAKVQWKVDNALQSGNSQESVTE | in bold. | |
| | QDSKDSTYSLSSTLTLSKADYEKHKVY | (www.imgt.org/ | |
| | ACEVTHQGLSSPVTKSFNRGEC (215) | mAb-DB/query | |
| | | Query: | |
| | | gantanerumab) | |

In one embodiment, polynucleotides encoding Gantenerumab, fragments or variants thereof may be used to prevent, treat or manage Alzheimer's Disease.

In one embodiment, polynucleotides encoding Gantenerumab, fragments or variants thereof may be used to prevent, treat or manage a disease or disorder associated with Amyloid-β. In some embodiments, the polynucleotides encoding Gantenerumab may be used to block formation of amyloid-β aggregation or to lead to increased production of microglia which contribute to the clearance of amyloid-β.

In one embodiment, polynucleotides encoding Gantenerumab, fragments or variants thereof may be used to prevent, treat or manage Alzheimer's Disease.

In one embodiment, polynucleotides encoding Gantenerumab, fragments or variants thereof may be used to prevent, treat or manage Lewy body dementia. Lewy body dementia is a type of dementia closely associated with Parkinson's disease. It is characterized anatomically by the presence of Lewy bodies, clumps of alpha-synuclein and ubiquitin protein in neurons, detectable in post mortem brain histology.

In one embodiment, polynucleotides encoding Gantenerumab, fragments or variants thereof may be used to prevent, treat or manage inclusion body myositis. Inclusion body myositis is an inflammatory muscle disease, characterized by slowly progressive weakness and wasting of both distal and proximal muscles, most apparent in the muscles of the arms and legs. There are two types: sporadic inclusion body myositis (sIBM) and hereditary inclusion body myopathy

In some embodiments, the polynucleotides encoding gantenerumab may be used to treat Alzheimer's disease and/or related diseases such as, but not limited to, Lewy body dementia, inclusion body myositis, cerebral amyloid angiopathy, and prion-based diseases such as classic Creutzfeldt-Jakob disease, new variant Creutzfeldt-Jakob disease Gerstmann-Sträussler-Scheinker syndrome, fatal familial insomnia and kuru.

### Solanezumab Parent Molecule or Antibody

In one embodiment, the polynucleotides of the present invention may encode Solanezumab, fragments or variants thereof.

Solanezumab, also known as LY2062430, is a humanized monoclonal IgG1 antibody directed against the mid-domain of the amyloid-β peptide. Solanezumab recognizes soluble monomeric (soluble), not fibrillar, amyloid-β and it may exert benefit by sequestering amyloid-β, shifting equilibria between different species of amyloid-β, and removing small soluble species of amyloid-β that are directly toxic to synaptic function.

Solanezumab is a humanized monoclonal antibody derived from the murine monoclonal antibody, m266. Solanezumab binds to the central, more hydrophobic region of the human amyloid-β peptide (against residues 16-24 of amyloid-β).

The three complementarity determining regions (CDR1, CDR 2 and CDR3) of the humanized light chain have amino acid sequences are derived from the corresponding CDRs of the mouse immunoglobulin light chain variable regions. Similarly, the three complementary determining regions (CDR1, CDR 2 and CDR3) of humanized heavy chain have amino acid sequences from the corresponding CDRs of the mouse immunoglobulin heavy chain variable regions. The constant regions are substantially from a human immunoglobulin. U.S. Pat. No. 7,195,761 (the contents of which are herein incorporated by reference in its entirety), assigned to Eli Lilly describes humanized heavy and light chains of a humanized anti-amyloid-β antibody based on the complementarity determining regions of murine anti-amyloid-β antibody m266.

In one embodiment, the polynucleotides described herein encode the Solanezumab antibody sequences, fragments or variants thereof described in U.S. Pat. No. 7,195,761, the contents of which are herein incorporated by reference in its entirety.

Certain sequences encoding the polynucleotide fragments, domains or heavy or light chains for Solanezumab are given in Table 56. The table is not an exhaustive list and any fragment or portion of the sequence which may be encoded in the polynucleotides of the invention.

TABLE 56
Table of Solanezumab Sequences

| | | | SEQ |
|---|---|---|---|
| | | | ID |
| Description | Sequence | Source | NO. |
| Heavy chain of | EVQLVESGGGLVQPGGSLRLSCAASGF | Immunogenetics | 271 |
| solanezumab | TFSRYSMSWVRQAPGKGLELVAQINSV | Information | |
| | GNSTYYPDTVKGRFTISRDNAKNTLYL | System; CHAIN | |
| | QMNSLRAEDTAVYYCASGDYWGQGTL | ID 9097_H. | |
| | VTVSSASTKGPSVFPLAPSSKSTSGGTA | CDRs are shown | |
| | ALGCLVKDYFPEPVTVSWNSGALTSGV | in bold. | |
| | HTFPAVLQSSGLYSLSSVVTVPSSSLGT | (www.imgt.org/ | |
| | QTYICNVNHKPSNTKVDKKVEPKSCDK | mAb-DB/query | |
| | THTCPPCPAPELLGGPSVFLFPPKPKDTL | Query: | |
| | MISRTPEVTCVVVDVSHEDPEVKFNWY | solanezumab) | |
| | VDGVEVHNAKTKPREEQYNSTYRVVS | | |
| | VLTVLHQDWLNGKEYKCKVSNKALPA | | |
| | PIEKTISKAKGQPREPQVYTLPPSRDELT | | |
| | KNQVSLTCLVKGFYPSDIAVEWESNGQ | | |
| | PENNYKTTPPVLDSDGSFFLYSKLTVDK | | |
| | SRWQQGNVFSCSVMHEALHNHYTQKS | | |
| | LSLSPGK (442) | | |
| Light chain of | DVVMTQSPLSLPVTLGQPASISCRSSQS | Immunogenetics | 272 |
| solanezumab | LIYSDGNAYLHWFLQKPGQSPRLLIYK | Information | |
| | VSNRFSGVPDRFSGSGSGTDFTLKISRV | System; CHAIN | |
| | EAEDVGVYYCSQSTHVPWTFGQGTKV | ID 9097_L. | |
| | EIKRTVAAPSVFIFPPSDEQLKSGTASVV | CDRs are shown | |
| | CLLNNFYPREAKVQWKVDNALQSGNS | in bold. | |
| | QESVTEQDSKDSTYSLSSTLTLSKADYE | (www.imgt.org/ | |
| | KHKVYACEVTHQGLSSPVTKSFNRGEC | mAb-DB/query | |
| | (219) | Query: | |
| | | solanezumab) | |

In one embodiment, polynucleotides encoding Solanezumab, fragments or variants thereof may be used to prevent, treat or manage Alzheimer's Disease.

In some embodiments, the polynucleotides encoding Solanezumab may be used to block formation of amyloid-β aggregation or to lead to increased production of microglia which contributes to the clearance of amyloid-β.

In some embodiments, the polynucleotides encoding Solanezumab may be used to treat Alzheimer's disease and/or related diseases such as of Lewy body dementia, inclusion body myositis, cerebral amyloid angiopathy, and prion-based diseases such as classic Creutzfeldt-Jakob disease, new variant Creutzfeldt-Jakob disease Gerstmann-Sträussler-Scheinker syndrome, fatal familial insomnia and kuru, either alone or in combination with other pharmaceutical agents. Said agents may be selected from the group consisting of donepezil, galantamine, rivastigmine, tacrine, and memantine (www.globalrph.com/alzheimers.htm; incorporated herein by reference in entirety), Huperzine A (Bai, D. L. Curr Med Chem. 2000, 7(3):355-74; incorporated herein by reference in entirety); and microtubule-stabilizing agents including taxanes, epothilones, discodermolide, laulimalide, peloruside A, cyclostreptin, taccalonolides, zampanolide, dactylolide, and ceratamines (Ballatore, C., J. Med. Chem. 2012, 55(21), 8979-96; the contents of which is incorporated herein by reference in its entirety).

In one embodiment, polynucleotides encoding Solanezumab, fragments or variants thereof may be used to prevent, treat or manage Lewy Body Dementia. Lewy body dementia is a type of dementia closely associated with Parkinson's disease. It is characterized anatomically by the presence of Lewy bodies, clumps of alpha-synuclein and ubiquitin protein in neurons, detectable in post mortem brain histology.

In one embodiment, polynucleotides encoding Solanezumab, fragments or variants thereof may be used to prevent, treat or manage Inclusion Body Myositis. Inclusion body myositis is an inflammatory muscle disease, characterized by slowly progressive weakness and wasting of both distal and proximal muscles, most apparent in the muscles of the arms and legs. There are two types: sporadic inclusion body myositis (sIBM) and hereditary inclusion body myopathy.

In one embodiment, polynucleotides encoding Solanezumab, fragments or variants thereof may be used to prevent, treat or manage Cerebral Amyloid Angiopathy. Cerebral amyloid angiopathy, also known as congophilic angiopathy, is a form of angiopathy in which amyloid deposits form in the walls of the blood vessels of the central nervous system.

In one embodiment, the polynucleotides encoding Solanezumab, fragments or variants thereof may be used to prevent, treat or manage Prion Diseases and disorder related to Prion Diseases. Prion diseases (also known as transmissible spongiform encephalopathies) are a group of progressive conditions (encephalopathies) that affect the brain and nervous system of many animals, including humans. According to the most widespread hypothesis they are transmitted by prions, though some other data suggest an involvement of a Spiroplasma infection. Mental and physical abilities deteriorate and a myriad of tiny holes appear in the cortex causing it to appear like a sponge (hence spongiform) when brain tissue obtained at autopsy is examined under a microscope. The disorders cause impairment of brain function, including memory changes, personality changes and problems with movement that worsen over time. Prion diseases of humans include classic Creutzfeldt-Jakob disease, new variant Creutzfeldt-Jakob disease (nvCJD, a human disorder related to bovine spongiform encephalopathy), Gerstmann-Sträussler-Scheinker syndrome, fatal familial insomnia, and kuru. These conditions form a spectrum of diseases with overlapping signs and symptoms.

### Factor IX-Fc Parent Molecule or Antibody

In one embodiment, the polynucleotides of the present invention may encode Factor IX-Fc, fragments or variants thereof.

Factor IX-Fc, also known as ALPROLIX™ and rFIXFc (recombinant factor IX fused to IgG1 Fc domain), is a bioengineered version of the blood coagulation factor IX, a protein needed for normal blood clotting. Factor IX-Fc is being developed by Biogen Idec in partnership with Swedish Orphan Biovitrum AB. Factor IX-Fc is a recombinant monomeric fusion protein composed of a single molecule of recombinant factor IX covalently fused to the human IgG1 Fc domain. Treatment with this agent produces increased circulating half-life and bleeding control in several species (see Shapiro, A. D. et al, Blood, 2012, 119(3), 666-671; and references cited therein, the contents of each of which is incorporated herein by reference in entirety).

Factor IX-Fc was prepared by fusing the protein known as coagulation factor IX to the Fc portion of immunoglobulin IgG1. While not wishing to be bound by theory, it is believed that this enables factor IX-Fc to use a naturally occurring pathway to prolong the time therapy remains in the body.

Factor IX Fc was originally developed by Syntonix Inc., which was later acquired by Biogen Idec. The design of Factor IX-Fc, methods of production thereof, amino acid and nucleic acid sequences thereof and methods for treatment of subjects in need of treatment of bleeding disorders are disclosed in U.S. Pat. Nos. 8,449,884, 8,329,182, 7,862,820, 7,404,956, 7,348,004 and in US Patent Publication Nos. 20130273047, 20130202595, 20130171175, 20130171138, 20110182919; the contents of each of which is incorporated herein by reference in entirety.

In one embodiment, the polynucleotides described herein encode Factor IX-Fc sequences, variant or fragments thereof described in U.S. Pat. Nos. 8,449,884, 8,329,182, 7,862,820, 7,404,956, 7,348,004 and in US Patent Publication Nos. 20130273047, 20130202595, 20130171175, 20130171138, 20110182919; the contents of each of which is incorporated herein by reference in entirety.

The factor IX portion of Factor IX-Fc has a primary amino acid sequence that is identical to the Thr148 allelic form of plasma-derived factor IX and has structural and functional properties similar to endogenous Factor IX. The Fc domain of factor IX-Fc contains the hinge, CH2, and CH3 regions of IgG1 (www.alprolix.com: prescribing information).

Certain sequences encoding the polynucleotide fragments, domains or heavy or light chains for Factor IX-Fc are given in Table 57. The table is not an exhaustive list and any fragment or portion of the sequence which may be encoded in the polynucleotides of the invention.

TABLE 57
Table of Factor IX-Fc Sequences

| | | | SEQ |
|---|---|---|---|
| | | | ID |
| Description | Sequence | Source | NO. |
| Factor-IX-Fc | MQRVNMIMAESPGLITICLLGYLLSAEC | U.S. Pat. | 273 |
| The signal | TVFLDHENANKILNRPKRYNSGKLEEF | Publication No. | |
| peptide is | VQGNLERECMEEKCSFEEAREVFENTE | 20050147618 | |
| underlined. | RTTEFWKQYVDGDQCESNPCLNGGSC | (SEQ ID NO: 23) | |
| The | KDDINSYECWCPFGFEGKNCELDVTCNI | | |
| propeptide is | KNGRCEQFCKNSADNKVVCSCTEGYRL | | |
| in bold. | AENQKSCEPAVPFPCGRVSVSQTSKLTR | | |
| The Fc domain | AETVFPDVDYVNSTEAETILDNITQSTQ | | |
| moiety is | SFNDFTRVVGGEDAKPGQFPWQVVLN | | |
| shown in bold | GKVDAFCGGSIVNEKWIVTAAHCVETG | | |
| and italics | VKITVVAGEHNIEETEHTEQKRNVIRIIP | | |
| | HHNYNAAINKYNHDIALLELDEPLVLN | | |
| | SYVTPICIADKEYTNIFLKFGSGYVSGW | | |
| | GRVFHKGRSALVLQYLRVPLVDRATCL | | |
| | RSTKFTIYNNMFCAGFHEGGRDSCQGD | | |
| | SGGPHVTEVEGTSFLTGIISWGEECAMK | | |
| | GKYGIYTKVSRYVNWIKEKTKLT [symbol] | | |
| | [symbol] | | |
| | [symbol] | | |
| | [symbol] | | |
| | [symbol] | | |
| | [symbol] | | |
| | [symbol] | | |
| | [symbol] | | |
| | [symbol] | | |
| | [symbol] | | |

In one embodiment, polynucleotides encoding Factor IX-Fc, fragments or variants thereof may be used to prevent, treat or manage hemophilia B. In one embodiment, the polynucleotides encoding Factor IX-FC, fragments or variants thereof may be used to prevent bleeding episodes, perioperative management, and routine prophylaxis to prevent or reduce the frequency of bleeding episodes.

In some embodiments, the polynucleotides encoding Factor IX-Fc may be used to replace deficient factor IX in individuals with hemophilia B (congenital Factor IX deficiency) for control and prevention of bleeding episodes, perioperative management, and routine prophylaxis to prevent or reduce the frequency of bleeding episodes.

In some embodiments, the polynucleotides of the present invention encoding Factor IX-Fc may be used to treat arthropathy, minor hemorrhage, hemarthroses, superficial muscle hemorrhage, soft tissue hemorrhage, moderate hemorrhage, intramuscle or soft tissue hemorrhage with dissection, mucous membrane hemorrhage, hematuria, major hemorrhage, hemorrhage of the pharynx, hemorrhage of the retropharynx, hemorrhage of the retroperitonium, hemorrhage of the central nervous system, bruises, cuts, scrapes, joint hemorrhage, nose bleed, mouth bleed, gum bleed, intracranial bleeding, intraperitoneal bleeding, minor spontaneous hemorrhage, bleeding after major trauma, moderate skin bruising, or spontaneous hemorrhage into joints, muscles, internal organs or the brain.

### Factor VIII-Fc Parent Molecule or Antibody

In one embodiment, the polynucleotides of the present invention may encode Factor VIII-Fc, fragments or variants thereof.

Factor VIII-Fc, also known as ELOCTATE™, BBB 031 and rFVIIIFc (recombinant factor VIII fused to IgG1 Fc), is a bioengineered version of the blood coagulation factor VIII, a protein needed for normal blood clotting.

Factor VIII-Fc is being developed by Biogen Idec in partnership with Swedish Orphan Biovitrum AB. Factor VIII-Fc is a recombinant monomeric fusion protein composed of a single molecule of recombinant factor VIII covalently fused to the human IgG1 Fc domain. The Fc domain enables the fusion protein to bind to the neonatal Fc receptor and protects it against intracellular degradation. Treatment with this agent produces increased circulating half-life and bleeding control in several species (Peters, R. T. et al., J. Thromb. Haemostasis, 2013, 11, 132-141; Dumont J. A. et al., Blood 2012; 119: 3024-30; and Powell J. S., et al. Blood 2012, 119: 3031-3037; and references cited therein, each of which is incorporated herein by reference in entirety).

Factor VIII-Fc is prepared by fusing an engineered version of the protein known as coagulation factor VIII to the Fc portion of immunoglobulin IgG1. It is believed that this enables factor VIII-Fc to use a naturally occurring pathway to prolong the time therapy remains in the body.

FVIII is synthesized as an approximately 300-kDa (2332 amino acids) single-chain (SC) protein that consists of the structural domains A1-A2-B-A3-C1-C2, including a large B domain with no known function or homology to other proteins. The B domain is normally processed intracellularly at various positions to generate a heavy chain (HC) (A1-A2-B) varying from 90 to 200 kDa in size and an 80-kDa light chain (LC) (A3-C1-C2) that remain associated via metal ion-mediated, non-covalent interactions. Deletion of a large portion of the B domain from Ser743 to Gln1638 has no effect on the functional activity of FVIII, but decreases the size of the protein significantly (38% reduction) to a 90-kDa HC and an 80-kDa LC, and increases FVIII expression levels in eukaryotic cells. This deletion is referred to as the B-domain deletion (BDD) and has been incorporated into Factor VIII-Fc (see Peters, R. T. et al., J. Thromb. Haemostasis, 2013, 11, 132-141 and references cited therein, each of which is incorporated herein by reference in entirety).

The design of Factor VIII-Fc, methods of production thereof, amino acid and nucleic acid sequences thereof and methods for treatment of subjects in need of treatment of bleeding disorders are disclosed in US Patent Publication Nos. 20130281671, 20130274194, 20130171138 and 20130108629; the contents of each of which is incorporated herein by reference in entirety.

In one embodiment, the polynucleotides described herein encode Factor VIII-Fc sequences, variant or fragments thereof described in US Patent Publication Nos. 20130281671, 20130274194, 20130171138 and 20130108629; the contents of each of which is incorporated herein by reference in entirety.

Certain sequences encoding the polynucleotide fragments, domains or heavy or light chains for Factor VIII-Fc are given in Table 58. The table is not an exhaustive list and any fragment or portion of the sequence which may be encoded in the polynucleotides of the invention.

TABLE 58
Table of Factor VIII-Fc Sequences

| | | | SEQ |
|---|---|---|---|
| | | | ID |
| Description | Sequence | Source | NO. |
| Factor-VIII-Fc | MQIELSTCFFLCLLRFCFSATRRYYLGA | PCT Publication | 274 |
| The signal | VELSWDYMQSDLGELPVDARFPPRVP | WO2011069164 | |
| peptide is | KSFPFNTSVVYKKTLFVEFTDHLFNIA | (SEQ ID NO: 2) | |
| underlined | KPRPPWMGLLGPTIQAEVYDTVVITL | | |
| (residues 1- | KNMASHPVSLHAVGVSYWKASEGAE | | |
| 20). | YDDQTSQREKEDDKVFPGGSHTYVVV | | |
| The heavy | QVLKENGPMASDPLCLTYSYLSHVDL | | |
| chain portion | VKDLNSGLIGALLVCREGSLAKEKTQ | | |
| of factor VIII | TLHKFILLFAVFDEGKSWHSETKNSL | | |
| is shown in | MQDRDAASARAWPKMHTVNGYVNR | | |
| bold (resides | SLPGLIGCHRKSVYWHVIGMGTTPEV | | |
| 20-753) | HSIFLEGHTFLVRNHRQASLEISPITFL | | |
| The. B domain | TAQTLLMDLGQFLLFCHISSHQHDG | | |
| portion is | MEAYVKVDSCPEEPQLRMKNNEEAE | | |
| underlined and | DYDDDLTDSEMDVVRFDDDNSPSFIQI | | |
| shown in | RSVAKKHPKTWVHYIAAEEEDWDYA | | |
| italics | PLVLAPDDRSYKSQYLNNGPQRIGRK | | |
| (residues 760- | YKKVRFMAYTDETFKTREAIQHESGI | | |
| 773. | LGPLLYGEVGDTLLIIFKNQASRPYNI | | |
| The Fc domain | YPHGITDVRPLYSRRLPKGVKHLKDF | | |
| moiety is | PILPGEIFKYKWTVTVEDGPTKSDPR | | |
| shown in bold | CLTRYYSSFVNMERDLASGLIGPLLIC | | |
| and italics | YKESVDQRGNQIMSDKRNVILFSVFD | | |
| (residues 760- | ENRSWYLTENIQRFLPNPAGVQLEDP | | |
| 773) | EFQASNIMHSINGYVFDSLQLSVCLHE | | |
| | VAYWYILSIGAQTDFLSVFFSGYTFKH | | |
| | KMVYEDTLTLFPFSGETVFMSMENP | | |
| | GLWILGCHNSDFRNRGMTALLKVSS | | |
| | CDKNTGDYYEDSYEDISAYLLSKNNAI | | |
| | EPRSFSQNPPVLKRHQREITRTTLQSDQE | | |
| | EIDYDDTISVEMKKEDFDIYDEDENQSP | | |
| | RSFQKKTRHYFIAAVERLWDYGMSSSP | | |
| | HVLRNRAQSGSVPQFKKVVFQEFTDGS | | |
| | FTQPLYRGELNEHLGLLGPYIRAEVEDN | | |
| | IMVTFRNQASRPYSFYSSLISYEEDQRQ | | |
| | GAEPRKNFVKPNETKTYFWKVQHHMA | | |
| | PTKDEFDCKAWAYFSDVDLEKDVHSG | | |
| | LIGPLLVCHTNTLNPAHGRQVTVQEFAL | | |
| | FFTIFDETKSWYFTENMERNCRAPCNIQ | | |
| | MEDPTFKENYRFHAINGYIMDTLPGLV | | |
| | MAQDQRIRWYLLSMGSNENIHSIHFSG | | |
| | HVFTVRKKEEYKMALYNLYPGVFETVE | | |
| | MLPSKAGIWRVECLIGEHLHAGMSTLF | | |
| | LVYSNKCQTPLGMASGHIRDFQITASGQ | | |
| | YGQWAPKLARLHYSGSINAWSTKEPFS | | |
| | WIKVDLLAPMIIHGIKTQGARQKFSSLYI | | |
| | SQFIIMYSLDGKKWQTYRGNSTGTLMV | | |
| | FFGNVDSSGIKHNIFNPPIIARYIRLHPTH | | |
| | YSIRSTLRMELMGCDLNSCSMPLGMES | | |
| | KAISDAQITASSYFTNMFATWSPSKARL | | |
| | HLQGRSNAWRPQVNNPKEWLQVDFQK | | |
| | TMKVTGVTTQGVKSLLTSMYVKEFLIS | | |
| | SSQDGHQWTLFFQNGKVKVFQGNQDS | | |
| | FTPVVNSLDPPLLTRYLRIHPQSWVHQI | | |
| | ALRMEVLGCEAQDLY [symbol] | | |
| | [symbol] | | |
| | [symbol] | | |
| | [symbol] | | |
| | [symbol] | | |
| | [symbol] | | |
| | [symbol] | | |
| | [symbol] | | |
| | [symbol] | | |

In one embodiment, polynucleotides encoding Factor VII-Fc, fragments or variants thereof may be used to prevent, treat or manage hemophilia A.

In some embodiments, the polynucleotides encoding factor-VIII-Fc may be used together with other drugs which may contain factor VIII (either recombinant or plasma-derived) as well as other clotting factors which may include but are not limited to von Willebrand factor. Pharmaceutical products falling within this category include, but are not limited to Alphanate® produced by Grifols, Humate-P® produced by CSL Behring, and Wilate®. In one aspect, these proteins can be prepared using the polynucleotides of the present invention.

In some embodiments, the polynucleotides encoding Factor VIII-Fc may be used to replace deficient factor VIII in individuals with hemophilia A (congenital Factor VIII deficiency) for control and prevention of bleeding episodes, perioperative management, and routine prophylaxis to prevent or reduce the frequency of bleeding episodes.

In some embodiments, the polynucleotides of the present invention encoding Factor VIII-Fc may be used as an antihemorrhagic agent in combination with other antihemorrhagic or hemostatic agents including, but not limited to, antifibrinolytics, fibrinogen, and vitamin K.

In some embodiments, the polynucleotides of the present invention encoding Factor VIII-Fc may be used to treat arthropathy, minor hemorrhage, hemarthroses, superficial muscle hemorrhage, soft tissue hemorrhage, moderate hemorrhage, intramuscle or soft tissue hemorrhage with dissection, mucous membrane hemorrhage, hematuria, major hemorrhage, hemorrhage of the pharynx, hemorrhage of the retropharynx, hemorrhage of the retroperitonium, hemorrhage of the central nervous system, bruises, cuts, scrapes, joint hemorrhage, nose bleed, mouth bleed, gum bleed, intracranial bleeding, intraperitoneal bleeding, minor spontaneous hemorrhage, bleeding after major trauma, moderate skin bruising, or spontaneous hemorrhage into joints, muscles, internal organs or the brain.

Naptumomab estafenatox Parent Molecule or Antibody

In one embodiment, the polynucleotides of the present invention may encode Naptumomab estafenatox, fragments or variants thereof.

Naptumomab estafenatox, also known as ANYARA™, ABR-217620 and TTS CD3, developed by Active Biotech AB is a fusion protein consisting of the antigen-binding fragment (Fab) of a monoclonal antibody with a genetically engineered version of superantigen staphylococcal enterotoxin A (SEA/E-120, “estafenatox”). The Fab binds to 5T4, an antigen expressed by various tumor cells. The superantigen induces an immune response by activating T lymphocytes.

Naptumomab estafenatox it is a fusion protein consisting of the antigen-binding fragment (Fab) of a murine monoclonal antibody known as 5T4 (WO1989007947; the contents of which is incorporated herein by reference in entirety), fused to the superantigen staphylococcal enterotoxin A (SEA/E-120, “estafenatox”). The Fab binds to 5T4, an antigen expressed by various tumor cells, and the superantigen induces an immune response by activating T lymphocytes (Forsberg, G. et al., J. Immunother. 2010, 33(5):492-9; Borghaei, H. et al., J. Clin. Oncology, 209, 27(25), 4116-4123); the contents of each of which is incorporated herein by reference in entirety).

Naptumomab estafenatox was developed from an earlier version of a similar agent ABR-214936 (Forsberg, G. Br. J. Cancer, 2001, 85(1), 129-135; the contents of which is incorporated herein by reference in entirety), and consists of a mutated variant of the superantigen SEA/E-120 (Erlandsson E, et al., J. Mol. Biol., 2003, 333, 893-905; the contents of which is incorporated herein by reference in entirety) linked to a Fab moiety of a murine monoclonal antibody recognizing 5T4 (Hole, N. et al., Br. J. Cancer, 1988, 57, 239-246; the contents of which is incorporated herein by reference in entirety). Naptumomab estafenatox is described in US Patent Publication No. 2006005711; the contents of which is incorporated herein by reference in entirety. The Fab includes the complementarity-determining regions (CDRs) of the murine antibody 5T4.

In one embodiment, the polynucleotides described herein encode Naptumomab estafenatox sequences, fragments or variants thereof described in US Patent Publication No. 2006005711; the contents of which is incorporated herein by reference in entirety.

Certain sequences encoding the polynucleotide fragments, domains or heavy or light chains for Naptumomab estafenatox are given in Table 59. The table is not an exhaustive list and any fragment or portion of the sequence which may be encoded in the polynucleotides of the invention.

TABLE 59
Table of Naptumomab estafenatox Sequences

| | | | SEQ |
|---|---|---|---|
| | | | ID |
| Description | Sequence | Source | NO. |
| 5T4Fab- | EVQLQQSGPDLVKPGASVKISCKASGY | SEQ ID NO: 7 of | 275 |
| SEA/E-120 | SFTGYYMHWVKQSPGKGLEWIGRINP | U.S. 20060057111. | |
| (ABR- | NNGVTLYNQKFKDKATLTVDKSSTTA | The SEA/E-120 | |
| 1217620) | YMELRSLTSEDSAVYYCARSTMITNYV | moiety is | |
| The heavy | MDYWGQGTSVTVSSAKTTPPSVYPLAP | underlined. | |
| chain sequence | GSAAQTNSMVTLGCLVKGYFPEPVTVT | The CDRs are | |
| precedes the | WNSGSLSSGVHTFPAVLQSDLYTLSSSV | shown in bold (as | |
| SEA/E-120 | TVPSSTWPSETVTCNVAHPASSTKVDK | identified in | |
| moiety. | KIVPRDSGGPSEKSEEINEKDLRKKSEL | Immunogenetics | |
| The light chain | QGTALGNLKQIYYYNSKAITSSEKSADQ | Information | |
| sequence is | FLTNTLLFKGFFTGHPWYNDLLVDLGS | System Chain | |
| appended to | TAATSEYEGSSVDLYGAYYGYQCAGG | Accession Nos. | |
| the end of the | TPNKTACMYGGVTLHDNNRLTEEKKV | 8588_H and | |
| underlined | PINLWIDGKQTTVPIDKVKTSKKEVTVQ | 8588_L | |
| SEA/E-120 | ELDLQARHYLHGKFGLYNSDSFGGKVQ | (www.imgt.org/ | |
| moiety. | RGLIVFHSSEGSTVSYDLFDAQGQYPDT | mAb-DB/query | |
| It is to be | LLRIYRDNTTISSTSLSISLYLYTTSIVMT | Naptumomab | |
| understood that | QTPTSLLVSAGDRVTITCKASQSVSNDV | estafenatox | |
| the light chain | AWYQQKPGQSPKLLISYTSSRYAGVPD | | |
| sequence is | RFSGSGYGTDFTLTISSVQAEDAAVYFC | | |
| associated with | QQDYNSPPTFGGGTKLEIKRADAAPTV | | |
| the heavy | SIFPPSSEQLTSGGASVVCFLNNFYPKDI | | |
| chain in a | NVKWKIDGSERQNGVLNSWTDQDSKD | | |
| normal Fab | STYSMSSTLTLTKDEYERHNSYTCEATH | | |
| arrangement. | KTSTSPIVKSFNRNES | | |
| SEA/E-120 | SEKSEEINEKDLRKKSELQGTALGNLK | SEQ ID NO: 3 of | 276 |
| | QIYYYNSKAITSSEKSADQFLTNTLLFK | U.S. 20060057111 | |
| | GFFTGHPWYNDLLVDLGSTAATSEYEG | | |
| | SSVDLYGAYYGYQCAGGTPNKTACMY | | |
| | GGVTLHDNNRLTEEKKVPINLWIDGKQ | | |
| | TTVPIDKVKTSKKEVTVQELDLQARHY | | |
| | LHGKFGLYNSDSFGGKVQRGLIVFHSSE | | |
| | GSTVSYDLFDAQGQYPDTLLRIYRDNT | | |
| | TISSTSLSISLYLYTT | | |

In one embodiment, polynucleotides encoding Naptumomab estafenatox, fragments or variants thereof may be used to prevent, treat or manage renal cell carcinoma. Renal cell carcinoma (RCC, formerly known as hypernephroma) is a kidney cancer that originates in the lining of the proximal convoluted tubule, one type of very small tubes in the kidney that transports waste molecules from the blood to the urine. RCC is the most common type of kidney cancer in adults, responsible for approximately 90-95% of cases. It has been described as one of the deadliest of cancers affecting the genitourinary tract.

In one embodiment, polynucleotides encoding Naptumomab estafenatox, fragments or variants thereof may be used to prevent, treat or manage non-small cell lung cancer. Non-small-cell lung carcinoma (NSCLC) is any type of epithelial lung cancer other than small cell lung carcinoma (SCLC). The most common types of NSCLC are squamous cell carcinoma, large cell carcinoma, and adenocarcinoma, but there are several other types that occur less frequently, and all types can occur in unusual histologic variants and as mixed cell-type combinations.

In one embodiment, polynucleotides encoding Naptumomab estafenatox, fragments or variants thereof may be used to prevent, treat or manage pancreatic cancer. Pancreatic cancer is a malignant neoplasm originating from transformed cells arising in tissues forming the pancreas. The most common type of pancreatic cancer, accounting for 95% of these tumors, is adenocarcinoma (tumors exhibiting glandular architecture on light microscopy) arising within the exocrine component of the pancreas. A minority of cases arise from islet cells, and are classified as neuroendocrine tumors. The signs and symptoms that eventually lead to the diagnosis depend on the location, the size, and the tissue type of the tumor, and may include abdominal pain, lower back pain, and jaundice (if the tumor compresses the bile duct), unexplained weight loss, and digestive problems.

In one embodiment, the polynucleotides encoding Naptumomab estafenatox may be used to activate T lymphocytes.

In one embodiment, the polynucleotides encoding Naptumomab estafenatox may be used to reduce harmful downstream events relating to alterations of cell adhesion, shape and motility which are influenced with aberrant expression of 5T4.

### Ramucirumab Parent Molecule or Antibody

In one embodiment, the polynucleotides of the present invention may encode Ramucirumab, fragments or variants thereof.

Ramucirumab, also known as IMC-1121B, is a human IgG1 antibody directed against vascular endothelial growth factor receptor 2 (VEGF2) developed by ImClone Systems Inc. Ramucirumab was designed to bind to a VEGFR-2 epitope involved in ligand binding, thereby blocking VEGF ligands from binding this site and preventing activation of the receptor (Lu et al., J. Biol. Chem., 2003, 278(44), 43496-43507, U.S. Pat. No. 7,498,414; the contents of each of which is incorporated herein by reference in their entirety). While not wishing to be bound by theory, inhibition of VEGF-stimulated VEGFR-2 activation by Ramucirumab confers significant antitumor activity in a range of malignancies in animal models as single agents and in combination with other therapeutics (Spratlin J. L., et al. J. Clin. Oncol. 2010, 28(5), 780-787; the contents of which is incorporated herein by reference in entirety).

Certain sequences encoding the polynucleotide fragments, domains or heavy or light chains for Ramucirumab are given in Table 60. The table is not an exhaustive list and any fragment or portion of the sequence which may be encoded in the polynucleotides of the invention.

TABLE 60
Table of Ramucirumab Sequences

| | | | SEQ |
|---|---|---|---|
| | | | ID |
| Description | Sequence | Source | NO. |
| Ramucirumab | EVQLVQSGGGLVKPGGSLRLSCAASGF | Immunogenetics | 277 |
| heavy chain | TFSSYSMNWVRQAPGKGLEWVSSISSS | Information | |
| | SSYIYYADSVKGRFTISRDNAKNSLYLQ | System; CHAIN | |
| | MNSLRAEDTAVYYCARVTDAFDIWGQ | ID 9098_H. | |
| | GTMVTVSSASTKGPSVFPLAPSSKSTSG | CDRs are shown | |
| | GTAALGCLVKDYFPEPVTVSWNSGALT | in bold. | |
| | SGVHTFPAVLQSSGLYSLSSVVTVPSSS | (www.imgt.org/ | |
| | LGTQTYICNVNHKPSNTKVDKKVEPKS | mAb-DBiquery | |
| | CDKTHTCPPCPAPELLGGPSVFLFPPKP | Query: | |
| | KDTLMISRTPEVTCVVVDVSHEDPEVK | ramucirumab) | |
| | FNWYVDGVEVHNAKTKPREEQYNSTY | | |
| | RVVSVLTVLHQDWLNGKEYKCKVSNK | | |
| | ALPAPIEKTISKAKGQPREPQVYTLPPSR | | |
| | EEMTKNQVSLTCLVKGFYPSDIAVEWE | | |
| | SNGQPENNYKTTPPVLDSDGSFFLYSKL | | |
| | TVDKSRWQQGNVFSCSVMHEALHNHY | | |
| | TQKSLSLSPGK (446) | | |
| Ramucirumab | DIQMTQSPSSVSASIGDRVTITCRASQGI | Immunogenetics | 278 |
| light chain | DNWLGWYQQKPGKAPKLLIYDASNLD | Information | |
| | TGVPSRFSGSGSGTYFTLTISSLQAEDFA | System; CHAIN | |
| | VYFCQQAKAFPPTFGGGTKVDIKGTV | ID 9098_L | |
| | AAPSVFIFPPSDEQLKSGTASVVCLLNN | (www.imgt.org/ | |
| | FYPREAKVQWKVDNALQSGNSQESVT | mAb-DB/query | |
| | EQDSKDSTYSLSSTLTLSKADYEKHKV | Query: | |
| | YACEVTHQGLSSPVTKSFNRGEC (214) | ramucirumab) | |
| Ramucirumab | EVQLVQSGGGLVKPGGSLRLSCAASGF | Immunogenetics | 279 |
| heavy chain | TFSSYSMNWVRQAPGKGLEWVSSISSS | Information | |
| variable | SSYIYYADSVKGRFTISRDNAKNSLYLQ | System; CHAIN | |
| domain | MNSLRAEDTAVYYCARVTDAFDIWGQ | ID 9098_H | |
| | GTMVTVSS (116) | CDRs are shown | |
| | | in bold. | |
| | | (www.imgt.org/ | |
| | | mAb-DB/query | |
| | | Query: | |
| | | ramucirumab) | |
| | | See also SEQ ID | |
| | | NO: 24 of U.S. | |
| | | Pat. 7,498,414 | |
| Ramucirumab | DIQMTQSPSSVSASIGDRVTITCRASQGI | Immunogenetics | 280 |
| light chain | DNWLGWYQQKPGKAPKLLIYDASNLD | Information | |
| variable | TGVPSRFSGSGSGTYFTLTISSLQAEDFA | System; CHAIN | |
| domain | VYFCQQAKAFPPTFGGGTKVDIK (107) | ID 9098_L | |
| | | CDRs are shown | |
| | | in bold. | |
| | | (www.imgt.org/ | |
| | | mAb-DB/query | |
| | | Query: | |
| | | ramucirumab) | |
| | | See also SEQ ID | |
| | | NO: 53 of U.S. | |
| | | Pat. 7,498,414 | |

In one embodiment, polynucleotides encoding Ramucirumab, fragments or variants thereof may be used to treat, prevent and/or reduce gastric adenocarcinoma. Gastric adenocarcinoma is a cancer of the stomach. It is often either asymptomatic (producing no noticeable symptoms) or it may cause only nonspecific symptoms (symptoms which are not specific to just gastric adenocarcinoma, but also to other related or unrelated disorders) in its early stages. By the time symptoms occur, the cancer has often reached an advanced stage (see below) and may have also metastasized (spread to other, perhaps distant, parts of the body), which is one of the main reasons for its relatively poor prognosis. Helicobacter pylori infection is the main risk factor in 65-80% of gastric cancers, but in only 2% of such infections. The mechanism by which H. pylori induces stomach cancer potentially involves chronic inflammation, or the action of H. pylori virulence factors such as CagA.

In one embodiment, polynucleotides encoding Ramucirumab, fragments or variants thereof may be used to treat, prevent and/or reduce non-small cell lung cancer. Non-small-cell lung carcinoma (NSCLC) is any type of epithelial lung cancer other than small cell lung carcinoma (SCLC). The most common types of NSCLC are squamous cell carcinoma, large cell carcinoma, and adenocarcinoma, but there are several other types that occur less frequently, and all types can occur in unusual histologic variants and as mixed cell-type combinations.

In one embodiment, polynucleotides encoding Ramucirumab, fragments or variants thereof may be used to treat, prevent and/or reduce hepatocellular carcinoma. Hepatocellular carcinoma (HCC, also called malignant hepatoma) is the most common type of liver cancer. Most cases of HCC are secondary to either a viral hepatitis infection (hepatitis B or C) or cirrhosis (alcoholism being the most common cause of hepatic cirrhosis). Treatment options of HCC and prognosis are dependent on many factors but especially on tumor size and staging. Tumor grade is also important. High-grade tumors will have a poor prognosis, while low-grade tumors may go unnoticed for many years, as is the case in many other organs.

In one embodiment, polynucleotides encoding Ramucirumab, fragments or variants thereof may be used to treat, prevent and/or reduce colorectal cancer. Colorectal cancer (also known as colon cancer, rectal cancer, bowel cancer or colorectal adenocarcinoma) is a cancer from uncontrolled cell growth in the colon or rectum (parts of the large intestine), or in the appendix. Genetic analysis shows that essentially colon and rectal tumors are genetically the same cancer. Symptoms of colorectal cancer typically include rectal bleeding and anemia which are sometimes associated with weight loss and changes in bowel habits. Most colorectal cancer occurs due to lifestyle and increasing age with only a minority of cases associated with underlying genetic disorders. It typically starts in the lining of the bowel and if left untreated, can grow into the muscle layers underneath, and then through the bowel wall. Screening is effective at decreasing the chance of dying from colorectal cancer and is recommended starting at the age of 50 and continuing until a person is 75 years old. Localized bowel cancer is usually diagnosed through sigmoidoscopy or colonoscopy.

In some embodiments, the polynucleotides encoding Ramucirumab may be used together with other antibodies specific for VEGFR2, including, but not limited to antibodies described in Zhu, Z. et al., Cancer Res. 1998, 58, 3209-3214; Zhu, Z. et al., Leukemia, 2003, 17, 604-611; and Prewett, M., et al., Cancer Res. 1999, 59, 5209-5218); each of which is incorporated herein by reference in entirety). In one aspect, these antibodies can be prepared using the polynucleotides of the present invention.

In some embodiments, the polynucleotides encoding Ramucirumab may be used to treat cancers or other hyperproliferative disorders arising from aberrations in angiogenesis migration, differentiation, tube formation, increase of vascular permeability, or maintenance of vascular integrity which arise as a result of dysregulated signaling mediated by VEGFR.

### Farletuzumab Parent Molecule or Antibody

In one embodiment, the polynucleotides of the present invention may encode Farletuzumab, fragments or variants thereof.

Farletuzumab, also known as MORAb-003, is a humanized IgG1 antibody directed against folate receptor a (FRA) developed by Morphotek Inc. While not wishing to be bound by theory, by binding to FRA, Farletuzumab triggers a host immune response against FRA-expressing cells, resulting in cell lysis. FRA is over-expressed on a number of epithelial-derived cancers such as ovarian, endometrial, breast, renal, lung, colorectal and pituitary.

Farletuzumab is a humanized IgG1 antibody with a molecular weight of 145 kDa. It contains human framework regions and the complementarity-determining regions (CDRs) of LK26, a murine antibody that binds to FRA. Farletuzumab is an optimized antibody which was generated by applying morphogenics technology to the original cell line producing the original suboptimal humanized LK26 antibody. After the optimization process, Farletuzumab was found to exhibit an affinity for FRA similar to that of the original murine LK26 antibody (approx. 2 nM) and a tissue binding profile consistent with the distribution of the folate receptor (see Ebel et al., Cancer Immunity, 2007, 7, 1-8 and references cited therein, the contents of each of which is incorporated herein by reference in entirety).

The light and heavy chains of Farletuzumab are described in US Patent Publication No. 20050232919 assigned to Morphotek, Inc.; the contents of each of which is incorporated by reference in its entirety. The three complementary determining regions (CDR1, CDR 2 and CDR3) of humanized light chain have amino acid sequences are derived from the corresponding CDRs of the mouse immunoglobulin light chain variable regions. Similarly, the three complementary determining regions (CDR1, CDR 2 and CDR3) of humanized heavy chain have amino acid sequences from the corresponding CDRs of the mouse immunoglobulin heavy chain variable regions and a variable region framework from a human heavy chain variable framework sequence. The constant regions are substantially from a human immunoglobulin.

In one embodiment, the polynucleotides described herein encode a Farletuzumab sequence, fragment or variant thereof described in US Patent Publication No. 20050232919, the contents of which are herein incorporated by reference in its entirety.

Certain sequences encoding the polynucleotide fragments, domains or heavy or light chains for Farletuzumab are given in Table 61. The table is not an exhaustive list and any fragment or portion of the sequence which may be encoded in the polynucleotides of the invention.

TABLE 61
Table of Farletuzumab Sequences

| | | | SEQ |
|---|---|---|---|
| | | | ID |
| Description | Sequence | Source | NO. |
| Farletuzumab | EVQLVESGGGVVQPGRSLRLSCSASGF | Immunogenetics | 281 |
| heavy chain | TFSGYGLSWVRQAPGKGLEWVAMISS | Information | |
| | GGSYTYYADSVKGRFAISRDNAKNTLF | System; CHAIN | |
| | LQMDSLRPEDTGVYFCARHGDDPAWF | ID 9067_H. | |
| | AYWGQGTPVTVSSASTKGPSVFPLAPSS | CDRs are shown | |
| | KSTSGGTAALGCLVKDYFPEPVTVSWN | in bold. | |
| | SGALTSGVHTFPAVLQSSGLYSLSSVVT | (www.imgt.org/ | |
| | VPSSSLGTQTYICNVNHKPSNTKVDKK | mAb-DB/query | |
| | VEPKSCDKTHTCPPCPAPELLGGPSVFL | Query: | |
| | FPPKPKDTLMISRTPEVTCVVVDVSHED | farletuzumab) | |
| | PEVKFNWYVDGVEVHNAKTKPREEQY | See also SEQ ID | |
| | NSTYRVVSVLTVLHQDWLNGKEYKCK | NO: 5 of U.S. | |
| | VSNKALPAPIEKTISKAKGQPREPQVYT | Pat. Publication | |
| | LPPSRDELTKNQVSLTCLVKGFYPSDIA | No. 20050232919 | |
| | VEWESNGQPENNYKTTPPVLDSDGSFF | | |
| | LYSKLTVDKSRWQQGNVFSCSVMHEA | | |
| | LHNHYTQKSLSLSPGK (449) | | |
| Farletuzumab | DIQLTQSPSSLSASVGDRVTITCSVSSSIS | Immunogenetics | 282 |
| light chain | SNNLHWYQQKPGKAPKPWIYGTSNLA | Information | |
| | SGVPSRFSGSGSGTDYTFTISSLQPEDIA | System; CHAIN | |
| | TYYCQQWSSYPYMYTFGQGTKVEIKR | ID 9067_L | |
| | TVAAPSVFIFPPSDEQLKSGTASVVCLL | CDRs are shown | |
| | NNFYPREAKVQWKVDNALQSGNSQES | in bold. | |
| | VTEQDSKDSTYSLSSTLTLSKADYEKHK | (www.imgt.org/ | |
| | VYACEVTHQGLSSPVTKSFNRGEC (217) | mAb-DB/query | |
| | | Query: | |
| | | farletuzumab) | |
| | | See also SEQ ID | |
| | | NO: 2 of U.S. | |
| | | Pat. Publication | |
| | | No. 20050232919 | |
| Farletuzumab | EVQLVESGGGVVQPGRSLRLSCSASGF | Immunogenetics | 283 |
| heavy chain | TFSGYGLSWVRQAPGKGLEWVAMISS | Information | |
| variable | GGSYTYYADSVKGRFAISRDNAKNTLF | System; CHAIN | |
| domain | LQMDSLRPEDTGVYFCARHGDDPAWF | ID 9067_H | |
| | AYWGQGTPVTVSS | CDRs are shown | |
| | | in bold. | |
| | | (www.imgt.org/ | |
| | | mAb-DB/query | |
| | | Query: | |
| | | farletuzumab) | |
| Farletuzumab | DIQLTQSPSSLSASVGDRVTITCSVSSSIS | Immunogenetics | 284 |
| light chain | SNNLHWYQQKPGKAPKPWIYGTSNLA | Information | |
| variable | SGVPSRFSGSGSGTDYTFTISSLQPEDIA | System; CHAIN | |
| domain | TYYCQQWSSYPYMYTFGQGTKVEIK | ID 9067_L | |
| | | CDRs are shown | |
| | | in bold. | |
| | | (www.imgt.org/ | |
| | | mAb-DB/query | |
| | | Query: | |
| | | farletuzumab) | |

In one embodiment, polynucleotides encoding Farletuzumab, fragments or variants thereof may be used to treat, prevent and/or reduce ovarian cancer. Ovarian cancer is a cancerous growth arising from the ovary. Symptoms are frequently very subtle early on and may include: bloating, pelvic pain, difficulty eating and frequent urination, and are easily confused with other illnesses. More than 90% of ovarian cancers are classified as epithelial cancers and are believed to arise from the surface (epithelium) of the ovary. However, some evidence suggests that the fallopian tube could also be the source of some ovarian cancers. Since the ovaries and tubes are closely related to each other, it is thought that these fallopian cancer cells can mimic ovarian cancer. Other types may arise from the egg cells (germ cell tumor) or supporting cells. Ovarian cancers are included in the category gynecologic cancer.

In some embodiments, the polynucleotides encoding Farletuzumab may be used together with other antibodies specific for FRA, including, but not limited to antibodies described in U.S. Pat. No. 8,557,966; incorporated herein by reference in entirety. In one aspect, these antibodies can be prepared using the polynucleotides of the present invention.

In one embodiment, the polynucleotides encoding Farletuzumab may be used to treat cancers or other hyperproliferative disorders arising from aberrations in signaling by FRA or aberrations in phosphorylation of proteins by Lyn kinase.

In some embodiments, the polynucleotides encoding Farletuzumab may be used as an anticancer agent, alone or in combination with other anticancer agents. Said agents may be selected from the group consisting of adriamycin PFS (doxorubicin hydrochloride) adriamycin RDF (doxorubicin hydrochloride), carboplatin, clafen (cyclophosphamide), cisplatin, cyclophosphamide, cytoxan (cyclophosphamide), doxorubicin hydrochloride, dox-SL (doxorubicin hydrochloride liposome), doxil (doxorubicin hydrochloride liposome), doxorubicin hydrochloride liposome, evacet (doxorubicin hydrochloride liposome), gemcitabine hydrochloride, gemzar (gemcitabine hydrochloride), hycamtin (topotecan hydrochloride), lipodox (doxorubicin hydrochloride liposome), neosar (cyclophosphamide), paclitaxel, paraplat (carboplatin), paraplatin (carboplatin), platinol (cisplatin), platinol-aq (cisplatin), taxanes, taxol (paclitaxel) and topotecan hydrochloride.

In one embodiment, polynucleotides encoding Farletuzumab, fragments or variants thereof may be used to treat, prevent and/or reduce pituitary adenoma, peritoneal neoplasms and adenocarcinoma of the lung.

In one embodiment, polynucleotides encoding Farletuzumab, fragments or variants thereof may be used to treat, prevent and/or reduce pituitary adenoma, peritoneal neoplasms and adenocarcinoma of the lung.

In one embodiment, polynucleotides encoding Farletuzumab, fragments or variants thereof may be used to treat, prevent and/or reduce pituitary adenoma, peritoneal neoplasms and adenocarcinoma of the lung.

### Obinutuzumab Parent Molecule or Antibody

In one embodiment, the polynucleotides of the present invention may encode Obinutuzumab, fragments or variants thereof.

Obinutuzumab, also known as GAZYVA™, is a type II recombinant humanized IgG1 anti-CD20 monoclonal antibody which selectivity binds to the extracellular domain of the human CD20 antigen on malignant human B cells (Robak, T., Curr. Opin. Invest. Drugs, 2009, 10(6), 588-596; the contents of which is herein incorporated by reference in entirety) developed by Genentech, a subsidiary of Hoffman-LaRoche. Obinutuzumab is glycoengineered and the Fc region of this monoclonal antibody is nonfucosylated, which gives it increased antibody-dependent cytotoxicity (ADCC) activity (Mössner, E. et al., Blood, 2010, 115(22), 4349-4402; the contents of which is herein incorporated by reference in entirety). The modifications in the GA101 structure were designed with the intention of providing a monoclonal antibody with increased B cell-killing activity compared with rituximab and other type I anti-CD20 mAbs (Salles, G. et al., Blood, 2012, 119(22), 5126-5132; the contents of which is herein incorporated by reference in entirety).

Obinutuzumab binds to CD20, an activated-glycosylated phosphoprotein expressed on the surface of all B-cells. This gene encodes a B-lymphocyte surface molecule that plays a role in the development and differentiation of B-cells into plasma cells. Its function is to enable optimal B-cell immune response, specifically against T-independent antigens (Kuijpers T. W. et al. J. Clin. Invest. 120 (1): 214-22, incorporated herein by reference in entirety).

The variable domains of Obinutuzumab are described in International Publication No. WO2005044859 assigned to Glycart Biotechnology AG; incorporated herein by reference in entirety. The three complementary determining regions (CDR1, CDR 2 and CDR3) of humanized light chain have amino acid sequences are derived from the corresponding CDRs of the mouse immunoglobulin light chain variable regions. Similarly, the three complementary determining regions (CDR1, CDR 2 and CDR3) of humanized heavy chain have amino acid sequences from the corresponding CDRs of the mouse immunoglobulin heavy chain variable regions and a variable region framework from a human heavy chain variable framework sequence. The constant regions are substantially from a human immunoglobulin. Obinutuzumab is the first Fc-engineered type II IgG1 antibody against CD20.

In one embodiment, the polynucleotides described herein encode an Obinutuzumab sequence, fragment and/or variant thereof described in International Publication No. WO2005044859, the contents of which are herein incorporated by reference in its entirety.

Certain sequences encoding the polynucleotide fragments, domains or heavy or light chains for Obinutuzumab are given in Table 62. The table is not an exhaustive list and any fragment or portion of the sequence which may be encoded in the polynucleotides of the invention.

TABLE 62
Table of Obinutuzumab Sequences

| | | | SEQ |
|---|---|---|---|
| | | | ID |
| Description | Sequence | Source | NO. |
| Obinutuzumab | QVQLVQSGAEVKKPGSSVKVSCKASG | Immunogenetics | 285 |
| heavy chain | YAFSYSWINWVRQAPGQGLEWMGRIF | Information | |
| | PGDGDTDYNGKFKGRVTITADKSTSTA | System; CHAIN | |
| | YMELSSLRSEDTAVYYCARNVFDGYW | ID 9043_H | |
| | LVYWGQGTLVTVSSASTKGPSVFPLAP | (www.imgt.org/ | |
| | SSKSTSGGTAALGCLVKDYFPEPVTVS | mAb-DB/query | |
| | WNSGALTSGVHTFPAVLQSSGLYSLSS | Query: | |
| | VVTVPSSSLGTQTYICNVNHKPSNTKVD | obinutuzumab) | |
| | KKVEPKSCDKTHTCPPCPAPELLGGPSV | | |
| | FLFPPKPKDTLMISRTPEVTCVVVDVSH | | |
| | EDPEVKFNWYVDGVEVHNAKTKPREE | | |
| | QYNSTYRVVSVLTVLHQDWLNGKEYK | | |
| | CKVSNKALPAPIEKTISKAKGQPREPQV | | |
| | YTLPPSRDELTKNQVSLTCLVKGFYPSD | | |
| | IAVEWESNGQPENNYKTTPPVLDSDGSF | | |
| | FLYSKLTVDKSRWQQGNVFSCSVMHE | | |
| | ALHNHYTQKSLSLSPGK | | |
| Obinutuzumab | DIVMTQTPLSLPVTPGEPASISCRSSKSL | Immunogenetics | 286 |
| light chain | LHSNGITYLYWYLQKPGQSPQLLIYQM | Information | |
| | SNLVSGVPDRFSGSGSGTDFTLKISRVE | System; CHAIN | |
| | AEDVGVYYCAQNLELPYTFGGGTKVE | ID 9043_L | |
| | IKRTVAAPSVFIFPPSDEQLKSGTASVVC | (www.imgt.org/ | |
| | LLNNFYPREAKVQWKVDNALQSGNSQ | mAb-DB/query | |
| | ESVTEQDSKDSTYSLSSTLTLSKADYEK | Query: | |
| | HKVYACEVTHQGLSSPVTKSFNRGEC | obinutuzumab) | |
| Obinutuzumab | QVQLVQSGAEVKKPGSSVKVSCKASG | Immunogenetics | 287 |
| heavy chain | YAFSYSWINWVRQAPGQGLEWMGRIF | Information | |
| variable | PGDGDTDYNGKFKGRVTITADKSTSTA | System; CHAIN | |
| domain | YMELSSLRSEDTAVYYCARNVFDGYW | ID 9043_H | |
| | LVYWGQGTLVTVSS | (www.imgt.org/m | |
| | | Ab-DB/query | |
| | | Query: | |
| | | obinutuzumab) | |
| | | See also SEQ ID | |
| | | NO: 40 of | |
| | | WO2005044859 | |
| Obinutuzumab | DIVMTQTPLSLPVTPGEPASISCRSSKSL | Immunogenetics | 288 |
| light chain | LHSNGITYLYWYLQKPGQSPQLLIYQM | Information | |
| variable | SNLVSGVPDRFSGSGSGTDFTLKISRVE | System; CHAIN | |
| domain | AEDVGVYYCAQNLELPYTFGGGTKVE | ID 9043_L | |
| | IK | (www.imgt.org/ | |
| | | mAb-DB/query | |
| | | Query: | |
| | | obinutuzumab) | |
| | | See also SEQ ID | |
| | | NO: 76 of | |
| | | WO2005044859 | |

In one embodiment, polynucleotides encoding Obinutuzumab, fragments or variants thereof may be used to treat, prevent and/or reduce Chronic Lymphocytic Leukemia (CLL). Chronic lymphoid leukemia is the most common type of leukemia in adults. Leukemias are cancers of the white blood cells (leukocytes). CLL affects B cell lymphocytes. B cells originate in the bone marrow, develop in the lymph nodes, and normally fight infection by producing antibodies. In CLL, B cells grow out of control and accumulate in the bone marrow and blood, where they crowd out healthy blood cells. CLL is a stage of small lymphocytic lymphoma, a type of B-cell lymphoma, which presents primarily in the lymph nodes. CLL and small lymphocytic lymphoma are considered manifestations of the same underlying disease with different appearances.

In some embodiments, the polynucleotides encoding Obinutuzumab may be used to block the triggering of complement-dependent cell lysis (CDCL) and antibody-dependent cell-mediated cytotoxicity (ADCC) of B-cells overexpressing CD20. Obinutuzumab may also be useful for reducing harmful downstream events relating to aberrant B-cell function.

In one embodiment, polynucleotides encoding Obinutuzumab, fragments or variants thereof may be used to treat, prevent and/or reduce non-Hodgkin's lymphoma, B cell lymphoma, and follicular lymphoma.

### Elotuzumab Parent Molecule or Antibody

In one embodiment, the polynucleotides of the present invention may encode Elotuzumab, fragments or variants thereof.

Elotuzumab, also known as HuLuc63, PDL-063, and BMS-901608, is a monoclonal antibody being developed by Bristol-Myers Squibb in collaboration with AbbVie. Humanization of MuLuc63 was carried out according to the procedure of Queen, C. et al. (PNAS (1989) 86: 10029-10033), the contents of which are herein incorporated by reference in their entirety. Human VH and VL segments with high homology to the MuLuc63 VH and VL amino acid sequences, respectively, were identified and the CDR sequences together with framework amino acids important for maintaining the structures of the CDRs were grafted into selected human framework sequences. The resulting humanized monoclonal antibody (HuLuc63) was expressed in the mouse myeloma cell line NSO.

Elotuzumab is produced by recombinant DNA technology, that targets a cell-surface protein called CS1, also known as CD2 subset 1, CRACC, SLAMF7, CD319, and 19A24, that is highly expressed on multiple myeloma cells.

While not wishing to be bound by theory, Elotuzumab can bind to the cell surface protein CS1 on the surface of both myeloma cells and natural killer (NK) cells. Elotuzumab enhances antibody directed cellular cytotoxicity (ADCC) of NK cells against multiple myeloma cells as described in Lonial et al., Journal of Clinical Oncology 2012, 1953-1959, the contents of which are herein incorporated by reference in their entirety.

In one embodiment, the polynucleotides described herein encode Elotuzumab sequences, fragments or variants thereof described in U.S. Pat. No. 7,709,610, the contents of which is herein incorporated by reference in its entirety.

Certain sequences encoding fragments, domains or heavy or light chains for Elotuzumab are given in Table 63. The table is not an exhaustive list and any fragment or portion of the sequence which may be encoded in the polynucleotides of the invention.

TABLE 63
Table of Elotuzumab Sequences

| | | | SEQ |
|---|---|---|---|
| | | | ID |
| Description | Sequence | Source | NO. |
| Elotuzumab | EVQLVESGGGLVQPGGSLRLSCAASGFDFS | CHEMBL1743010 | 289 |
| heavy chain | RYWMSWVRQAPGKGLEWIGEINPDSSTIN | | |
| | YAPSLKDKFIISRDNAKNSLYLQMNSLRAE | | |
| | DTAVYYCARPDGNYWYFDVWGQGTLVTV | | |
| | SSASTKGPSVFPLAPSSKSTSGGTAALGCLV | | |
| | KDYFPEPVTVSWNSGALTSGVHTFPAVLQS | | |
| | SGLYSLSSVVTVPSSSLGTQTYICNVNHKPS | | |
| | NTKVDKKVEPKSCDKTHTCPPCPAPELLGG | | |
| | PSVFLFPPKPKDTLMISRTPEVTCVVVDVSH | | |
| | EDPEVKFNWYVDGVEVHNAKTKPREEQY | | |
| | NSTYRVVSVLTVLHQDWLNGKEYKCKVS | | |
| | NKALPAPIEKTISKAKGQPREPQVYTLPPSR | | |
| | DELTKNQVSLTCLVKGFYPSDIAVEWESNG | | |
| | QPENNYKTTPPVLDSDGSFFLYSKLTVDKS | | |
| | RWQQGNVFSCSVMHEALHNHYTQKSLSLS | | |
| | PGK | | |
| Elotuzumab | DIQMTQSPSSLSASVGDRVTITCKASQDVGI | CHEMBL1743010 | 290 |
| light chain | AVAWYQQKPGKVPKLLIYWASTRHTGVPD | | |
| | RFSGSGSGTDFTLTISSLQPEDVATYYCQQY | | |
| | SSYPYTFGQGTKVEIKRTVAAPSVFIFPPSD | | |
| | EQLKSGTASVVCLLNNFYPREAKVQWKVD | | |
| | NALQSGNSQESVTEQDSKDSTYSLSSTLTLS | | |
| | KADYEKHKVYACEVTHQGLSSPVTKSFNR | | |
| | GEC | | |
| HuLuc-63 | EVQLVESGGGLVQPGGSLRLSCAASGFDFS | From U.S. Pat. | 291 |
| Variable | RYWMSWVRQAPGKGLEWIGEINPDSSTIN | No. 7,709,610, | |
| Heavy Chain | YAPSLKDKFIISRDNAKNSLYLQMNSLRAE | SEQ ID NO: 47 | |
| | DTAVYYCARPDGNYWYFDVWGQGTLVTV | | |
| | SS | | |
| HuLuc-63 | DIQMTQSPSSLSASVGDRVTITCKASQDVGI | From U.S. Pat. | 292 |
| Variable | AVAWYQQKPGKVPKLLIYWASTRHTGVPD | No. 7,709,610 | |
| Light Chain | RFSGSGSGTDFTLTISSLQPEDVATYYCQQY | SEQ ID NO: 50 | |
| | SSYPYTFGQGTKVEIK | | |
| HuLuc-63 | RYWMS | From U.S. Pat. | 293 |
| HL CDR1 | | No. 7,709,610 | |
| | | SEQ ID NO: 30 | |
| HuLuc-63 | EINPDSSTINYAPSLKD | From U.S. Pat. | 294 |
| HL CDR2 | | No. 7,709,610 | |
| | | SEQ ID NO: 31, | |
| | | 33 | |
| HuLuc-63 | PDGNYWYFDV | From U.S. Pat. | 295 |
| HL CDR3 | | No. 7,709,610 | |
| | | SEQ ID NO: 32 | |
| HuLuc-63 | KASQDVGIAVA | From U.S. Pat. | 296 |
| VL CDR1 | | No. 7,709,610 | |
| | | SEQ ID NO: 35 | |
| HuLuc-63 | WASTRHT | From U.S. Pat. | 297 |
| VL CDR2 | | No. 7,709,610 | |
| | | SEQ ID NO: 36 | |
| HuLuc-63 | QQYSSYPYT | From U.S. Pat. | 298 |
| VL CDR3 | | No. 7,709,610 | |
| | | SEQ ID NO: 37 | |

In one embodiments, the polynucleotides encoding Elotuzumab may be used for treating both relapsed/refractory and multiple myeloma. In one embodiment, the polynucleotides may encode Elotuzumab to kill myeloma cells.

In one embodiment, polynucleotides encoding Elotuzumab, fragments or variants thereof may be used to treat or prolong the refractory period following treatment for multiple myeloma. As a non-limiting example, the polynucleotides may be used to treat patients who are no longer responding to other forms of treatment.

Inotuzumab ozogamicin Parent Molecule or Antibody

In one embodiment, the polynucleotides of the present invention may encode Inotuzumab ozogamicin, fragments or variants thereof.

Inotuzumab ozogamicin, also known as CMC-544, developed and manufactured by Wyeth is a humanized IgG4 anti-CD22 monoclonal antibody (mAb), G5/44, covalently linked to CalichDMH via an acid-labile 4-(4′-acetylphenoxy) butanoic acid (AcBut) linker (DiJoseph et al., Blood. 2004 103: 1807-1814) the contents of each of which are herein incorporated by reference in their entirety. Inotuzumab ozogamicin is an antibody drug conjugate (ADC) comprising the humanized monoclonal antibody Inotuzumab, conjugated to N-acetyl-γ-calicheamicin dimethyl hydrazide (CalichDMH), a cytotoxic agent from the class of calicheamicins.

Inotuzumab ozogamicin was derived from the murine anti-CD22 mAb m5/44, in a process which involved the grafting of the murine complementary determining regions (CDRs) into the human antibody framework (See e.g., Beeler et al., J Virol. 1989, 63, 2941-2950, U.S. Pat. Nos. 5,824,307 and 5,824,307 and also EP 0783525; the contents of each of which are herein incorporated by reference in their entirety).

Inotuzumab ozogamicin is produced by recombinant DNA technology wherein it is expressed and purified from Chinese hamster ovary (CHO) cells. In an intermediate step, Inotuzumab is covalently linked to CalichDMH. The ADC Inotuzumab ozogamicin targets and binds CD22 antigen on B-cells, it is absorbed into the cell, and the cytotoxic CalichDMH is released to destroy the cell.

While not wishing to be bound by theory, Inotuzumab ozogamicin can bind to the CD22 expressed on malignant B-cells, CD22 being known in the art as a cellular receptor that actively binds ligands and promotes uptake by the host. Antibody drug conjugates against cellular receptors with specific expression on malignant cells may be useful for treatment of CD22+ B-cell malignancies. In one embodiment, polynucleotides encoding Inotuzumab ozogamicin, fragments or variants thereof may be used for active killing of CD22 expressing cancer cells.

Certain sequences encoding fragments, domains or heavy or light chains for Inotuzumab ozogamicin are given in Table 64. The table is not an exhaustive list and any fragment or portion of the sequence which may be encoded in the polynucleotides of the invention.

TABLE 64
Table of Inotuzumab Ozogamicin Sequences

| | | | SEQ |
|---|---|---|---|
| | | | ID |
| Description | Sequence | Source | NO. |
| Heavy Chain | MDFGFSLVFLALILKGVQCEVQLVQSGAEVK | From U.S. | 299 |
| | KPGASVKVSCKASGYRFTNYWIHWVRQAPG | Pat. No. | |
| | QGLEWIGGINPGNNYATYRRKFQGRVTMTA | 8,153,768 | |
| | DTSTSTVYMELSSLRSEDTAVYYCTREGYGN | SEQ ID NO. | |
| | YGAWFAYWGQGTLVTVSSASTKGPSVFPLA | 30 | |
| | PCSRSTSESTAALGCLVKDYFPEPVTVSWNSG | | |
| | ALTSGVHTFPAVLQSSGLYSLSSVVTVPSSSL | | |
| | GTKTYTCNVDHKPSNTKVDKRVESKYGPPCP | | |
| | PCPAPEFLGGPSVFLFPPKPKDTLMISRTPEVT | | |
| | CVVVDVSQEDPEVQFNWYVDGVEVHNAKT | | |
| | KPREEQFNSTYRVVSVLTVLHQDWLNGKEY | | |
| | KCKVSNKGLPSSIEKTISKAKGQPREPQVYTL | | |
| | PPSQEEMTKNQVSLTCLVKGFYPSDIAVEWE | | |
| | SNGQPENNYKTTPPVLDSDGSFFLYSRLTVD | | |
| | KSRWQEGNVFSCSVMHEALHNHYTQKSLSL | | |
| | SLGK | | |
| Heavy chain | EVQLVQSGAEVKKPGASVKVSCKASGYRFT | From U.S. | 300 |
| Variable | NYWIHWVRQAPGQGLEWIGGINPGNNYATY | Pat. No. | |
| Region | RRKFQGRVTMTADTSTSTVYMELSSLRSEDT | 8,153,768 | |
| | AVYYCTREGYGNYGAWFAYWGQGTLVTVSS | SEQ ID NO. | |
| | | 27 | |
| Heavy-chain- | NYWIH | From U.S. | 301 |
| CDR1 | | Pat. No. | |
| | | 8,153,768 | |
| | | SEQ ID NO. 1 | |
| Heavy-chain- | GINPGNNYATYRRKFQG | From U.S. | 302 |
| CDR2 | | Pat. No. | |
| | | 8,153,768 | |
| | | SEQ ID NO. | |
| | | 27 (a.a. 50-66) | |
| Heavy-chain- | EGYGNYGAWFAY | From U.S. | 303 |
| CDR3 | | Pat. No. | |
| | | 8,153,768 | |
| | | SEQ ID NO. 3 | |
| Light Chain | MKLPVRLLVLLLFWIPASRGDVQVTQSPSSLS | From U.S. | 304 |
| | ASVGDRVTITCRSSQSLANSYGNTFLSWYLH | Pat. No. | |
| | KPGKAPQLLIYGISNRFSGVPDRFSGSGSGTD | 8,153,768 | |
| | FTLTISSLQPEDFATYYCLQGTHQPYTFGQGT | SEQ ID NO. | |
| | KVEIKRTVAAPSVFIFPPSDEQLKSGTASVVC | 28 | |
| | LLNNFYPREAKVQWKVDNALQSGNSQESVT | | |
| | EQDSKDSTYSLSSTLTLSKADYEKHKVYACE | | |
| | VTHQGLSSPVTKSFNRGEC | | |
| Light chain | DVQVTQSPSSLSASVGDRVTITCRSSQSLANS | From U.S. | 305 |
| Variable | YGNTFLSWYLHKPGKAPQLLIYGISNRFSGVP | Pat. No. | |
| Region | DRFSGSGSGTDFTLTISSLQPEDFATYYCLQG | 8,153,768 | |
| | THQPYTFGQGTKVEIKR | SEQ ID NO. | |
| | | 19 | |
| Light chain | RSSQSLANSYGNTFLS | From U.S. | 306 |
| CDR1 | | Pat. No. | |
| | | 8,153,768 | |
| | | SEQ ID NO. 4 | |
| Light chain | GISNRFS | From U.S. | 307 |
| CDR2 | | Pat. No. | |
| | | 8,153,768 | |
| | | SEQ ID NO. 5 | |
| Light chain | LQGTHQPYT | From U.S. | 308 |
| CDR3 | | Pat. No. | |
| | | 8,153,768 | |
| | | SEQ ID NO. 6 | |

In one embodiment, the polynucleotides described herein encode Inotuzumab ozogamicin may be used to treat B-cell malignancies, such as, but not limited to, non-Hodgkin Lymphoma and relapsed or acute lymphoblastic leukemia. As used herein the term “B-cell malignancies” refers to a population of diseases characterized by CD22+ B-cells, including a sub-population of non-Hodgkin's lymphomas and acute lymphoblastic leukemias. Non-Hodgkin's lymphoma refers to any of a large group of cancers of lymphocytes (white blood cells). Non-Hodgkin lymphomas can occur at any age and are often marked by lymph nodes that are larger than normal, fever, and weight loss. There are many different types of non-Hodgkin lymphoma. These types can be divided into aggressive (fast-growing) and indolent (slow-growing) types, and they can be formed from either B-cells or T-cells. B-cell non-Hodgkin lymphomas include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. T-cell non-Hodgkin lymphomas include mycosis fungoides, anaplastic large cell lymphoma, and precursor T-lymphoblastic lymphoma. Lymphomas that occur after bone marrow or stem cell transplantation are usually B-cell non-Hodgkin lymphomas.

In one embodiment, polynucleotides encoding Inotuzumab ozogamicin, fragments or variants thereof may be used to target a sub-population CD22+ B-cells of cancer cells in a multi-malignant cell type cancer in conjunction with other cancer treatments. As a non-limiting example, the polynucleotides encoding Inotuzumab ozogamicin may be used to kill CD22+ B-cells in a leukemia characterized by both malignant B-cells and T-cells.

### Moxetumomab Pasudotox Parent Molecule or Antibody

In one embodiment, the polynucleotides of the present invention may encode Moxetumomab Pasudotox, fragments or variants thereof.

Moxetumomab pasudotox, also known as GCR-8015, was developed by Genecor which was acquired by Cambridge Antibody Technology and renamed CAT-8015. Moxetumomab pasudotox is a recombinant immunotoxin consisting of the Fv portion of the anti-CD22 antibody CAT-8015 covalently fused to a 38 KDa fragment of Pseudomonas exotoxin-A (PE38). The light chain and heavy chain of the humanized murine CD22 variable regions are joined by a disulfide bond between cysteines engineered into the framework region, to form a disulfide-stabilized antibody fragment. (U.S. Pat. No. 7,982,011, the contents of which are herein incorporated by reference in their entirety).

Moxetumomab pasudotox is produced by recombinant DNA technology wherein the sequence for the CD22 binding antibody fragment is cloned in frame with a fragment of Pseudomonas exotoxin-A. Moxetumomab pasudotox is expressed and purified from a suitable expression system as is commonly known in the art, including but not limited to COS, CHO, HeLa and myeloma cell lines. Moxetumomab pasudotox targets and binds CD22 antigen on B-cells, it is absorbed into the cell, and the cytotoxic Pseudomonas exotoxin-A is released to destroy the cell. Pseudomonas exotoxin-A induces caspase-mediated apoptosis via a mechanism involving mitochondrial damage and blocks translational elongation by binding to elongation factor 2.

While not wishing to be bound by theory, Moxetumomab pasudotox can bind to the CD22 expressed on malignant B-cells, CD22 being known in the art as a cellular receptor that actively binds ligands and promotes uptake by the host. Antibody drug conjugates against cellular receptors with specific expression on malignant cells may be useful for treatment of CD22+ B-cell malignancies. In one embodiment, polynucleotides encoding Moxetumomab pasudotox, fragments or variants thereof may be used for active killing of CD22 expressing cancer cells.

In one embodiment, the polynucleotides described herein encode the Moxetumomab pasudotox sequences, fragments or variants thereof described in U.S. Pat. No. 7,982,011, the contents of which are herein incorporated by reference in their entirety.

Certain sequences encoding fragments, domains or heavy or light chains for Moxetumomab pasudotox are given in Table 65. The table is not an exhaustive list and any fragment or portion of the sequence which may be encoded in the polynucleotides of the invention.

TABLE 65
Table of Moxetumomab Pasudotox Sequences

| | | | SEQ |
|---|---|---|---|
| | | | ID |
| Description | Sequence | Source | NO. |
| VH region | EVQLVESGGGLVKPGGSLKLSCAASGFAFS | From U.S. Pat. | 309 |
| | IYDMSWVRQTPEKRLEWVAYISSGGGTTY | No. 7,982,011 | |
| | YPDTVKGRFTISRDNAKNTLYLQMSSLKSE | SEQ ID NO. 21 | |
| | DTAMYYCARHSGYGTHWGVLFAYWGQG | | |
| | TLVTVSA | | |
| Heavy chain | MEVQLVESGGGLVKPGGSLKLSCAASGFA | CHEMBL1743043 | 310 |
| | FSIYDMSWVRQTPEKCLEWVAYISSGGGTT | | |
| | YYPDTVKGRFTISRDNAKNTLYLQMSSLKS | | |
| | EDTAMYYCARHSGYGTHWGVLFAYWGQ | | |
| | GTLVTVSAKASGGPEGGSLAALTAHQACH | | |
| | LPLETFTRHRQPRGWEQLEQCGYPVQRLV | | |
| | ALYLAARLSWNQVDQVIRNALASPGSGGD | | |
| | LGEAIREQPEQARLALTLAAAESERFVRQG | | |
| | TGNDEAGAANGPADSGDALLERNYPTGAE | | |
| | FLGDGGDVSFSTRGTQNWTVERLLQAHRQ | | |
| | LEERGYVFVGYHGTFLEAAQSIVFGGVRA | | |
| | RSQDLDAIWRGFYIAGDPALAYGYAQDQE | | |
| | PDARGRIRNGALLRVYVPRSSLPGFYRTSL | | |
| | TLAAPEAAGEVERLIGHPLPLRLDAITGPEE | | |
| | EGGRLETILGWPLAERTVVIPSAIPTDPRNV | | |
| | GGDLDPSSIPDKEQAISALPDYASQPGKPPR | | |
| | EDLK | | |
| Heavy-chain- | GFAFSIYD | From U.S. Pat. | 311 |
| CDR1 | | No. 7,982,011 | |
| | | SEQ ID NO. 13 | |
| Heavy-chain- | ISSGGGTT | From U.S. Pat. | 312 |
| CDR2 | | No. 7,982,011 | |
| | | SEQ ID NO. 14 | |
| Heavy-chain- | ARHSGYGTHWGVLFAY | From U.S. Pat. | 313 |
| CDR3 | | No. 7,982,011 | |
| | | SEQ ID NO. 16 | |
| VL Region | DIQMTQTTSSLSASLGDRVTISCRASQDIHG | From U.S. Pat. | 314 |
| | YLNWYQQKPDGTVKLLIYYTSILHSGVPSR | No. 7,982,011 | |
| | FSGSGSGTDYSLTISNLEQEDFATYFCQQG | SEQ ID NO. 20 | |
| | NTLPWTFGGGTKLEIK | | |
| VL Region | DIQMTQTTSSLSASLGDRVTISCRASQDISN | From U.S. Pat. | 315 |
| | YLNWYQQKPDGTVKLLIYYTSILHSGVPSR | No. 7,982,011 | |
| | FSGSGSGTDYSLTISNLEQEDFATYFCQQG | SEQ ID NO. 2 | |
| | NTLPWTFGGGTKLEIK | | |
| Light chain | MDIQMTQTTSSLSASLGDRVTISCRASQDIS | CHEMBL1743043 | 316 |
| | NYLNWYQQKPDGTVKLLIYYTSILHSGVPS | | |
| | RFSGSGSGTDYSLTISNLEQEDFATYFCQQ | | |
| | GNTLPWTFGCGTKLEIK | | |
| Light chain | QDIHGY | From U.S. Pat. | 317 |
| CDR1 | | No. 7,982,011 | |
| | | SEQ ID NO. 7 | |
| Light chain | YTS | From U.S. Pat. | — |
| CDR2 | | No. 7,982,011 | |
| | | SEQ ID NO. 11 | |
| Light chain | QQGNTLPWT | From U.S. Pat. | 318 |
| CDR3 | | No. 7,982,011 | |
| | | SEQ ID NO. 12 | |

In one embodiment, the polynucleotides described herein encode Moxetumomab pasudotox and may be used in the treatment of B-cell malignancies, such as, but not limited to, Non-Hodgkin Lymphoma and relapsed or acute lymphoblastic leukemia.

In one embodiment, polynucleotides encoding Moxetumomab pasudotox, fragments or variants thereof may be used to target a sub-population CD22+ B-cells of cancer cells in a multi-malignant cell type cancer in conjunction with other cancer treatments. As a non-limiting example, the polynucleotides may be used to kill CD22+ B-cells in a leukemia characterized by both malignant B-cells and T-cells.

### Necitumumab Parent Molecule or Antibody

In one embodiment, the polynucleotides of the present invention may encode Necitumumab, fragments or variants thereof.

Necitumumab, also known as IMC-11F8, is developed and manufactured by Eli Lilly. Necitumumab is a fully human monoclonal antibody composed of fragments isolated from a human naïve Fab bacteriophage library cloned using common techniques known in the art onto a fully human IgG1 framework. (See e.g., Beeler et al., J Virol. 1989, 63, 2941-2950, U.S. Pat. Nos. 5,824,307 and 5,824,307 and also EP 0783525; the contents of each of which are herein incorporated by reference in their entirety).

Necitumumab is produced by recombinant DNA technology, directed to an epitope in the ligand binding site of epidermal growth factor receptor (EGFR). Necitumumab targets the ligand binding site of EGFR, thereby preventing receptor activation and signaling. While not wishing to be bound by theory, necitumumab can bind to the ligand binding site of EGFR which can prevent activation and signaling of the receptor.

In one embodiment, the polynucleotides described herein encode Necitumumab sequences, fragments or variants thereof described in U.S. Pat. No. 7,598,350, the contents of which are herein incorporated by reference in its entirety.

Certain sequences encoding fragments, domains or heavy or light chains for Necitumumab are given in Table 66. The table is not an exhaustive list and any fragment or portion of the sequence which may be encoded in the polynucleotides of the invention.

TABLE 66
Table of Necitumumab Sequences

| | | | SEQ ID |
|---|---|---|---|
| Description | Sequence | Source | NO. |
| VH region | QVQLQESGPGLVKPSQTLSLTCTVSGGSISS | From U.S. | 319 |
| | GDYYWSWIRQPPGKGLEWIGYIYYSGSTDY | Pat. No. | |
| | NPSLKSRVTMSVDTSKNQFSLKVNSVTAAD | 7,598,350 SEQ | |
| | TAVYYCARVSIFGVGTFDYWGQGTLVTVSS | ID NO. 8 | |
| Heavy chain | QVQLQESGPGLVKPSQTLSLTCTVSGGSISS | CHEMBL1743047 | 320 |
| | GDYYWSWIRQPPGKGLEWIGYIYYSGSTDY | | |
| | NPSLKSRVTMSVDTSKNQFSLKVNSVTAAD | | |
| | TAVYYCARVSIFGVGTFDYWGQGTLVTVSS | | |
| | ASTKGPSVLPLAPSSKSTSGGTAALGCLVKD | | |
| | YFPEPVTVSWNSGALTSGVHTFPAVLQSSGL | | |
| | YSLSSVVTVPSSSLGTQTYICNVNHKPSNTK | | |
| | VDKRVEPKSCDKTHTCPPCPAPELLGGPSVF | | |
| | LFPPKPKDTLMISRTPEVTCVVVDVSHEDPE | | |
| | VKFNWYVDGVEVHNAKTKPREEQYNSTYR | | |
| | VVSVLTVLHQDWLNGKEYKCKVSNKALPA | | |
| | PIEKTISKAKGQPREPQVYTLPPSREEMTKN | | |
| | QVSLTCLVKGFYPSDIAVEWESNGQPENNY | | |
| | KTTPPVLDSDGSFFLYSKLTVDKSRWQQGN | | |
| | VFSCSVMHEALHNHYTQKSLSLSPGK | | |
| Heavy-chain- | SGDYYWS | From U.S. | 321 |
| CDR1 | | Pat. No. | |
| | | 7,598,350 SEQ | |
| | | ID NO. 2 | |
| Heavy-chain- | YIYYSGSTDYNPSLKS | From U.S. | 322 |
| CDR2 | | Pat. No. | |
| | | 7,598,350 SEQ | |
| | | ID NO. 4 | |
| Heavy-chain- | VSIFGVGTFDY | From U.S. | 323 |
| CDR3 | | Pat. No. | |
| | | 7,598,350 SEQ | |
| | | ID NO. 6 | |
| Light chain | EIVMTQSPATLSLSPGERATLSCRASQSVSSY | CHEMBL1743047 | 324 |
| | LAWYQQKPGQAPRLLIYDASNRATGIPARF | | |
| | SGSGSGTDFTLTISSLEPEDFAVYYCHQYGS | | |
| | TPLTFGGGTKAEIKRTVAAPSVFIFPPSDEQL | | |
| | KSGTASVVCLLNNFYPREAKVQWKVDNAL | | |
| | QSGNSQESVTEQDSKDSTYSLSSTLTLSKAD | | |
| | YEKHKVYACEVTHQGLSSPVTKSFNRGEC | | |
| VL Region | EIVMTQSPATLSLSPGERATLSCRASQSVSSY | From U.S. | 325 |
| | LAWYQQKPGQAPRLLIYDASNRATGIPARF | Pat. No. | |
| | SGSGSGTDFTLTISSLEPEDFAVYYCHQYGS | 7,598,350 SEQ | |
| | TPLTFGGGTKAEIK | ID NO. 16 | |
| Light chain | RASQSVSSYLA | From U.S. | 326 |
| CDR1 | | Pat. No. | |
| | | 7,598,350 SEQ | |
| | | ID NO. 10 | |
| Light chain | DASNRAT | From U.S. | 327 |
| CDR2 | | Pat. No. | |
| | | 7,598,350 SEQ | |
| | | ID NO. 12 | |
| Light chain | HQYGSTPLT | From U.S. | 328 |
| CDR3 | | Pat. No. | |
| | | 7,598,350 SEQ | |
| | | ID NO. 14 | |

In one embodiment, the polynucleotides described herein encode Necitumumab and may be used in the treatment of nonsquamous and squamous non-small-cell lung cancer (NSCLC). The most common types of NSCLC are squamous cell carcinoma, large cell carcinoma, and adenocarcinoma, but there are several other types that occur less frequently, and all types can occur in unusual histologic variants and as mixed cell-type combinations.

In one embodiment, polynucleotides encoding necitumumab, fragments or variants thereof may be used to treat non-small cell lung cancer which may be caused by aberrant EGFR mediated signaling pathways. As a non-limiting example, the polynucleotides may be used to treat patients who may be resistant to conventional chemotherapy and/or cancer therapeutics.

In some embodiments, the polynucleotides encoding necitumumab, fragments or variants thereof may be used for the prophylaxis, diagnosis and/or treatment of EGFR linked cancers in a subject. In some aspects, the subject is a patient at high risk of morbidity and mortality, including, but not limited to, patients who have a cancer resistant to other modes of treatment.

### Rilotumumab Parent Molecule or Antibody

In one embodiment, the polynucleotides of the present invention may encode Rilotumumab, fragments or variants thereof.

Rilotumumab, also known as AMG-102, is a human IgG2 monoclonal antibody developed and manufactured by Amgen which derived Rilotumumab from XENOMOUSE® mice (Abgenix, Fremont, Calif.). Hybridomas are generated from the mice and screened for binding to the human hepatocyte growth factor (HGF). The selected antibody heavy and light chain variable regions are cloned by molecular techniques common in the art and grafted to a human IgG2 antibody framework (See e.g., Beeler et al., J Virol. 1989, 63, 2941-2950, U.S. Pat. Nos. 5,824,307 and 5,824,307 and also EP 0783525; the contents of each of which are herein incorporated by reference in their entirety).

Rilotumumab is produced by recombinant DNA technology, directed to an epitope of human hepatocyte growth factor (HGF). Rilotumumab targets the c-Met binding site of HGF, inhibiting its ligation to and activation of c-Met signal transduction of downstream signaling pathways.

While not wishing to be bound by theory, Rilotumumab can bind HGF which prevents binding and activation of c-Met receptors. Rilotumumab exhibits neutralizing and clearance activity against HGF with a binding affinity of approximately 19 pmol/L. Linear and time-invariant kinetics over a dose range of 0.5-20 mg/kg showed a typical systemic clearance and central volume of distribution of 0.184 L/day and 3.56 L, respectively. (see e.g. Burgess et al. Mol Cancer Ther 2010; 9:400-409; and Zhu et al., Journal of Pharmaceutical Sciences 2014 103:328-336, the contents of each of which are herein incorporated by reference in their entirety).

In one embodiment, the polynucleotides described herein encode Rilotumumab sequences, fragments or variants thereof described in U.S. Pat. No. 8,609,090, the contents of which are herein incorporated by reference in its entirety.

Certain sequences encoding fragments, domains or heavy or light chains for Rilotumumab are given in Table 67. The table is not an exhaustive list and any fragment or portion of the sequence which may be encoded in the polynucleotides of the invention.

TABLE 67
Table of Rilotumumab Sequences

| | | | SEQ |
|---|---|---|---|
| | | | ID |
| Description | Sequence | Source | NO. |
| Heavy chain | QVQLQESGPGLVKPSETLSLTCTVSGGSISIY | From U.S. | 329 |
| | YWSWIRQPPGKGLEWIGYVYYSGSTNYNPS | Pat. No. | |
| | LKSRVTISVDTSKNQFSLKLNSVTAADTAVY | 8,609,090 SEQ | |
| | YCARGGYDFWSGYFDYWGQGTLVTVSSAS | ID NO. 39 | |
| | TKGPSVFPLAPCSRSTSESTAALGCLVKDYF | | |
| | PEPVTVSWNSGALTSGVHTFPAVLQSSGLYS | | |
| | LSSVVTVPSSNFGTQTYTCNVDHKPSNTKV | | |
| | DKTVERKCCVECPPCPAPPVAGPSVFLFPPK | | |
| | PKDTLMISRTPEVTCVVVDVSHEDPEVQFN | | |
| | WYVDGVEVHNAKTKPREEQFNSTFRVVSV | | |
| | LTVVHQDWLNGKEYKCKVSNKGLPAPIEKT | | |
| | ISKTKGQPREPQVYTLPPSREEMTKNQVSLT | | |
| | CLVKGFYPSDIAVEWESNGQPENNYKTTPP | | |
| | MLDSDGSFFLYSKLTVDKSRWQQGNVFSCS | | |
| | VMHEALHNHYTQKSLSLSPGK | | |
| Heavy-chain- | IYYWS | From U.S. | 330 |
| CDR1 | | Pat. No. | |
| | | 8,609,090 SEQ | |
| | | ID NO. 97 | |
| Heavy-chain- | YVYYSGSTNYNPSLKS | From U.S. | 331 |
| CDR2 | | Pat. No. | |
| | | 8,609,090 SEQ | |
| | | ID NO. 107 | |
| Heavy-chain- | GGYDFWSGYFDY | From U.S. | 332 |
| CDR3 | | Pat. No. | |
| | | 8,609,090 SEQ | |
| | | ID NO. 117 | |
| Light chain | EIVMTQSPATLSVSPGERATLSCRASQSVDS | From U.S. | 333 |
| | NLAWYRQKPGQAPRLLIYGASTRATGIPAR | Pat. No. | |
| | FSGSGSGTEFTLTISSLQSEDFAVYYCQQYIN | 8,609,090 SEQ | |
| | WPPITFGQGTRLEIKRTVAAPSVFIFPPSDEQ | ID NO. 38 | |
| | LKSGTASVVCLLNNFYPREAKVQWKVDNA | | |
| | LQSGNSQESVTEQDSKDSTYSLSSTLTLSKA | | |
| | DYEKHKVYACEVTHQGLSSPVTKSFNRGEC | | |
| Light chain | RASQSVDSNLA | From U.S. | 334 |
| CDR1 | | Pat. No. | |
| | | 8,609,090 SEQ | |
| | | ID NO. 67 | |
| Light chain | GASTRAT | From U.S. | 335 |
| CDR2 | | Pat. No. | |
| | | 8,609,090 SEQ | |
| | | ID NO. 77 | |
| Light chain | QQYINWPPIT | From U.S. | 336 |
| CDR3 | | Pat. No. | |
| | | 8,609,090 SEQ | |
| | | ID NO. 87 | |

In one embodiment, polynucleotides Rilotumumab may used to neutralize c-Met signaling and may be useful for treatment of cancers characterized by mutated or aberrant expression of c-Met.

In one embodiment, polynucleotides encoding Rilotumumab, fragments or variants thereof may be used for treatment of cancers including, but not limited to, breast cancer, colorectal cancer, gastric carcinoma, glioma, head and neck squamous cell carcinoma, hereditary and sporadic papillary renal carcinoma, leukemia, lymphoma, Li-Fraumeni syndrome, malignant pleural mesothelioma, melanoma, multiple myeloma, non-small cell lung carcinoma, osteosarcoma, ovarian cancer, pancreatic cancer, prostate cancer, small cell lung cancer, synovial sarcoma, thyroid carcinoma, and transitional cell carcinoma of urinary bladder.

In some embodiments, the polynucleotides encoding Rilotumumab may be used to treat epithelial cancer.

### Onartuzumab Parent Molecule or Antibody

In one embodiment, polynucleotides of the present invention may encode Onartuzumab, fragments or variants thereof.

Onartuzumab, also known as METMAB®, is a humanized monovalent monoclonal antibody targeting hepatocyte growth factor receptor c-Met. Onartuzumab is developed and manufactured by Genentech, a subsidiary of Roche under the brand name METMAB®. The c-Met receptor is a receptor tyrosine kinase and is found in abundance on a variety of cancer cell surfaces where it is thought to contribute to aberrant proliferation. Onartuzumab binding to this receptor is intended to block hepatocyte growth factor (HGF) ligand binding and subsequent signal transduction as a means of counteracting neoplastic activity and resulting in c-Met-expressing tumor cell death.

Onartuzumab comprises an Fab antibody fragment developed through humanization and affinity maturation of a monoclonal antibody, 5D5. Onartuzumab further comprises an Fc region that has been specifically engineered for large-scale assembly in E. coli by the inclusion of “knob” and “hole” domains to promote self-assembly (See e.g., Merchant, M. et al., 2013. PNAS. E2987-96; the contents of each of which are herein incorporated by reference in their entirety).

In one embodiment, the polynucleotides described herein encode Onartuzumab sequences, fragments and variants thereof described in U.S. Pat. No. 7,476,724 and US Patent Publication No. US20130004484, the contents of each of which are herein incorporated by reference in its entirety.

Certain sequences encoding fragments, domains or heavy or light chains for Onartuzumab are given in Table 68. The table is not an exhaustive list and any fragment or portion of the sequences listed may be encoded in the polynucleotides of the invention.

TABLE 68
Table of Onartuzumab Sequences

| | | | SEQ |
|---|---|---|---|
| | | | ID |
| Description | Sequence | Source | NO. |
| VH region | EVQLVESGGGLVQPGGSLRLSCAASGYTFT | From | 337 |
| | SYWLHWVRQAPGKGLEWVGMIDPSNSDT | US20130004484 | |
| | RFNPNFKDRFTISADTSKNTAYLQMNSLRA | SEQ ID NO. | |
| | EDTAVYYCATYRSYVTPLDYWGQGTLVTV | 19 | |
| | SS | | |
| Heavy chain | EVQLVESGGGLVQPGGSLRLSCAASGYTFT | CHEMBL1743051 | 338 |
| | SYWLHWVRQAPGKGLEWVGMIDPSNSDT | | |
| | RFNPNFKDRFTISADTSKNTAYLQMNSLRA | | |
| | EDTAVYYCATYRSYVTPLDYWGQGTLVTV | | |
| | SSASTKGPSVFPLAPSSKSTSGGTAALGCLV | | |
| | KDYFPEPVTVSWNSGALTSGVHTFPAVLQS | | |
| | SGLYSLSSVVTVPSSSLGTQTYICNVNHKPS | | |
| | NTKVDKKVEPKSCDKTHTCPPCPAPELLGG | | |
| | PSVFLFPPKPKDTLMISRTPEVTCVVVDVSH | | |
| | EDPEVKFNWYVDGVEVHNAKTKPREEQY | | |
| | NSTYRVVSVLTVLHQDWLNGKEYKCKVSN | | |
| | KALPAPIEKTISKAKGQPREPQVYTLPPSRE | | |
| | EMTKNQVSLSCAVKGFYPSDIAVEWESNG | | |
| | QPENNYKTTPPVLDSDGSFFLVSKLTVDKS | | |
| | RWQQGNVFSCSVMHEALHNHYTQKSLSLS | | |
| | PGK | | |
| Heavy-chain- | GYTFTSYWLH | From | 339 |
| CDR1 | | US20130004484 | |
| | | SEQ ID NO. | |
| | | 4, U.S. Pat. No. | |
| | | 7,476,724 SEQ | |
| | | ID NO. 191 | |
| Heavy-chain- | GMIDPSNSDTRFNPNFKD | From | 340 |
| CDR2 | | US20130004484 | |
| | | SEQ ID NO. | |
| | | 5, U.S. Pat. No. | |
| | | 7,476,724 SEQ | |
| | | ID NO. 192 | |
| Heavy-chain- | ATYRSYVTPLDY | From | 341 |
| CDR3 | | US20130004484 | |
| | | SEQ ID NO. | |
| | | 6, U.S. Pat. No. | |
| | | 7,476,724 SEQ | |
| | | ID NO. 193 | |
| VL Region | DIQMTQSPSSLSASVGDRVTITCKSSQSLLY | From | 342 |
| | TSSQKNYLAWYQQKPGKAPKLLIYWASTR | US20130004484 | |
| | ESGVPSRFSGSGSGTDFTLTISSLQPEDFATY | SEQ ID NO. | |
| | YCQQYYAYPWTFGQGTKVEIKR | 20 | |
| Light chain | DIQMTQSPSSLSASVGDRVTITCKSSQSLLY | CHEMBL1743051; | 343 |
| | TSSQKNYLAWYQQKPGKAPKLLIYWASTR | PDB: | |
| | ESGVPSRFSGSGSGTDFTLTISSLQPEDFATY | 4K3J_L | |
| | YCQQYYAYPWTFGQGTKVEIKRTVAAPSV | | |
| | FIFPPSDEQLKSGTASVVCLLNNFYPREAKV | | |
| | QWKVDNALQSGNSQESVTEQDSKDSTYSL | | |
| | SSTLTLSKADYEKHKVYACEVTHQGLSSPV | | |
| | TKSFNRGEC | | |
| Light chain | KSSQSLLYTSSQKNYLA | From | 344 |
| CDR1 | | US20130004484 | |
| | | SEQ ID NO. | |
| | | 1, U.S. Pat. No. | |
| | | 7,476,724 SEQ | |
| | | ID NO. 183 | |
| Light chain | WASTRES | From | 345 |
| CDR2 | | US20130004484 | |
| | | SEQ ID NO. | |
| | | 2, U.S. Pat. No. | |
| | | 7,476,724 SEQ | |
| | | ID NO. 184 | |
| Light chain | QQYYAYPWT | From | 346 |
| CDR3 | | US20130004484 | |
| | | SEQ ID NO. | |
| | | 3, U.S. Pat. No. | |
| | | 7,476,724 SEQ | |
| | | ID NO. 185 | |

In one embodiment, the polynucleotides of the invention encoding Onartuzumab, fragments and variants thereof may be used to treat multiple forms of cancer. In some cases, polynucleotides may be used to reduce or eliminate the presence of cancer cells in subjects, where such cancer cells express the c-Met receptor. Such cancers may include, but are not limited to non-small cell lung cancer (NSCLC), glioblastoma, pancreatic cancer and gastric cancer. In some cases, polynucleotides of the invention may be administered in combination with erlotinib (TARCEVA®) for the treatment of NSCLC.

In one embodiment, the polynucleotides described herein encodes Onartuzumab, fragments and variants thereof may be used to treat and/or prevent gastric adenocarcinoma. Gastric adenocarcinoma is a cancer of the stomach. It is often either asymptomatic (producing no noticeable symptoms) or it may cause only nonspecific symptoms (symptoms which are not specific to just gastric adenocarcinoma, but also to other related or unrelated disorders) in its early stages. By the time symptoms occur, the cancer has often reached an advanced stage and may have also metastasized (spread to other, perhaps distant, parts of the body), which is one of the main reasons for its relatively poor prognosis. Helicobacter pylori infection is the main risk factor in 65-80% of gastric cancers, but in only 2% of such infections. The mechanism by which H. pylori induces stomach cancer potentially involves chronic inflammation, or the action of H. pylori virulence factors such as CagA.

In one embodiment, the polynucleotides described herein encodes Onartuzumab, fragments and variants thereof may be used to treat and/or prevent non-small-cell lung carcinoma (NSCLC).

### Lambrolizumab Parent Molecule or Antibody

In one embodiment, polynucleotides of the present invention may encode Lambrolizumab, fragments or variants thereof.

Lambrolizumab, also known as MK-3475, is a humanized monoclonal antibody (IgG4) composed of human and murine antibody amino acid sequences and is developed and manufactured by Merck & Co.

Lambrolizumab targets human programmed cell death 1 (PD-1) on the surface of activated T-cells. PD-1 acts as a negative regulator of T-cell activity upon binding of ligands hPD-L1 or hPD-L2. Cancer cells have been shown to express either or both of the ligands for PD-1, thereby inhibiting activation of T-cells and subsequent cytotoxic activity. Binding of Lambrolizumab to the PD-1 receptor prevents hPD-L1 and hPD-L2 binding, thereby preventing inhibition of T-cell cytotoxic activity and allowing for targeted destruction of cancer cells.

While not wishing to be bound by theory, Lambrolizumab can bind to PD-1 receptor on the surface of activated T-cells. In both animal model and clinical studies, PD-1 promotes tolerance of ‘self’ in tissues, a mechanism ‘adaptive resistance’ exploited by tumors that express ligands to PD-1 to mask their cancerous nature (see e.g. Merelli et al. Crit Rev Oncol Hematol. 2014 January; 89(1):140-65; the contents of which are herein incorporated by reference in their entirety).

Lambrolizumab comprises an immunoglobulin G4, anti-(human programmed cell death 1); humanized mouse monoclonal [228-L-proline (H10-S>P)]γ4 heavy chain (134-218′)-disulfide with humanized mouse monoclonal κ light chain dimer (226-226″:229-229″) bisdisulfide (U.S. Pat. No. 8,354,509; the contents of each of which are herein incorporated by reference in their entirety).

In one embodiment, the polynucleotides described herein encodes Lambrolizumab, fragments and variants thereof described in U.S. Pat. No. 8,354,509, the contents of each of which are herein incorporated by reference in their entirety.

Certain sequences encoding fragments, domains or heavy or light chains for Lambrolizumab are given in Table 69. The table is not an exhaustive list and any fragment or portion of the sequence which may be encoded in the polynucleotides of the invention.

TABLE 69
Table of Lambrolizumab Sequences

| | | | SEQ |
|---|---|---|---|
| | | | ID |
| Description | Sequence | Source | NO. |
| Heavy chain | QVQLVQSGVEVKKPGASVKVSCKASGY | From U.S. Pat. | 347 |
| | TFTNYYMYWVRQAPGQGLEWMGGINP | No. 8,354,509 | |
| | SNGGTNFNEKFKNRVTLTTDSSTTTAYM | SEQ ID NO. 31 | |
| | ELKSLQFDDTAVYYCARRDYRFDMGFD | | |
| | YWGQGTTVTVSSASTKGPSVFPLAPCSR | | |
| | STSESTAALGCLVKDYFPEPVTVSWNSG | | |
| | ALTSGVHTFPAVLQSSGLYSLSSVVTVPS | | |
| | SSLGTKTYTCNVDHKPSNTKVDKRVESK | | |
| | YGPPCPPCPAPEFLGGPSVFLFPPKPKDTL | | |
| | MISRTPEVTCVVVDVSQEDPEVQFNWY | | |
| | VDGVEVHNAKTKPREEQFNSTYRVVSV | | |
| | LTVLHQDWLNGKEYKCKVSNKGLPSSIE | | |
| | KTISKAKGQPREPQVYTLPPSQEEMTKN | | |
| | QVSLTCLVKGFYPSDIAVEWESNGQPEN | | |
| | NYKTTPPVLDSDGSFFLYSRLTVDKSRW | | |
| | QEGNVFSCSVMHEALHNHYTQKSLSLSL | | |
| | GK | | |
| Light Chain | EIVLTQSPATLSLSPGERATLSCRASKGV | From U.S. Pat. | 348 |
| | STSGYSYLHWYQQKPGQAPRLLIYLASY | No. 8,354,509 | |
| | LESGVPARFSGSGSGTDFTLTISSLEPEDF | SEQ ID NO. 36 | |
| | AVYYCQHSRDLPLTFGGGTKVEIKRTVA | | |
| | APSVFIFPPSDEQLKSGTASVVCLLNNFY | | |
| | PREAKVQWKVDNALQSGNSQESVTEQD | | |
| | SKDSTYSLSSTLTLSKADYEKHKVYACE | | |
| | VTHQGLSSPVTKSFNRGEC | | |
| Heavy- | NYYMY | From U.S. Pat. | 349 |
| chain-CDR1 | | No. 8,354,509 | |
| | | SEQ ID NO. 18 | |
| Heavy- | GINPSNGGTNFNEKFKN | From U.S. Pat. | 350 |
| chain-CDR2 | | No. 8,354,509 | |
| | | SEQ ID NO. 19 | |
| Heavy- | RDYRFDMGFDY | From U.S. Pat. | 351 |
| chain-CDR3 | | No. 8,354,509 | |
| | | SEQ ID NO. 20 | |
| Light chain | RASKGVSTSGYSYLH | From U.S. Pat. | 352 |
| CDR1 | | No. 8,354,509 | |
| | | SEQ ID NO. 15 | |
| Light chain | LASYLES | From U.S. Pat. | 353 |
| CDR2 | | No. 8,354,509 | |
| | | SEQ ID NO. 16 | |
| Light chain | QHSRDLPLT | From U.S. Pat. | 354 |
| CDR3 | | No. 8,354,509 | |
| | | SEQ ID NO. 17 | |

In one embodiment, the polynucleotides described herein encode Lambrolizumab and may be used in the treatment of advanced or metastatic renal carcinoma, melanoma, multiple myeloma, non-small cell lung carcinoma, leukemia, and solid tumors.

In one embodiment, the polynucleotides described herein encode Lambrolizumab and may be used in the treatment of non-small-cell lung carcinoma (NSCLC) in patients with tumors expressing PD-1.

### Chimeric Monoclonal Antibody 14.18 Parent Molecule or Antibody

In one embodiment, the polynucleotides of the present invention may encode CH14.18, fragments or variants thereof.

Chimeric Monoclonal Antibody 14.18 (CH14.18) is a chimeric monoclonal antibody that binds to the ganglioside GD2 and induces antibody-dependent cell-mediated cytotoxicity and complement-dependent cytotoxicity against GD2-expressing tumor cells (as described in Mueller, et al., 1990, the content of which is herein incorporated by reference in its entirety). GD2 is overexpressed in malignant melanoma, neuroblastoma, osteosarcoma, and small cell carcinoma of the lung.

In one embodiment, the polynucleotides described herein encode a CH14.18 sequence, fragment and/or variant thereof described in U.S. Pat. No. 8,470,991, the contents of which are herein incorporated by reference in its entirety. In one embodiment, the polynucleotides described herein encode a light chain variable region CH14.18 sequence as SEQ ID NO: 1 in U.S. Pat. No. 8,470,991, the contents of which are herein incorporated by reference in its entirety. In one embodiment, the polynucleotides described herein encode a heavy chain variable region CH14.18 sequence as SEQ ID NO: 2 in U.S. Pat. No. 8,470,991, the contents of which are herein incorporated by reference in its entirety.

Certain sequences encoding the polynucleotide fragments, domains or heavy or light chains for CH14.18 are given in Table 70. The table is not an exhaustive list and any fragment or portion of the sequence which may be encoded in the polynucleotides of the invention.

TABLE 70
Table of CH14.18 Sequences

| | | | SEQ |
|---|---|---|---|
| | | | ID |
| Description | Sequence | Source | NO. |
| Light chain | EVQLVQSGAEVEKPGASVKISCKASGSS | From U.S. Pat. | 355 |
| variable region | FTGYNMNWVRQNIGKSLEWIGAIDPYY | No. U.S. Pat. No. | |
| | GGTSYNQKFKGRATLTVDKSTSTAYM | 8,470,991 | |
| | HLKSLRSEDTAVYYCVSGMEYWGQGT | SEQ ID NO: 1 | |
| | SVTVSS | | |
| Heavy chain | DVVMTQTPLSLPVTPGEPASISCRSSQSL | From U.S. Pat. | 356 |
| variable region | VHRNGNTYLHWYLQKPGQSPKLLIHKV | No. U.S. Pat. No. | |
| | SNRFSGVPDRFSGSGSGTDFTLKISRVE | 8,470,991 | |
| | AEDLGVYFCSQSTHVPPLTFGAGTKLELK | SEQ ID NO: 2 | |

In one embodiment, polynucleotides encoding CH14.18, fragments or variants thereof may be used to treat and/or prevent Neuroblastoma. Neuroblastoma is a cancer arising in immature nerve cells, is the most common cancer in infants, the most common extracranial solid tumor of childhood, and the third most common cancer in children.

In one embodiment, polynucleotides encoding CH14.18, fragments or variants thereof may be used to treat and/or prevent Melanoma.

In one embodiment, polynucleotides encoding Ch14.18 fragments or variants thereof may be used in the treatment of cancers, including but not limited to, neuroblastoma, melanoma, soft tissue sarcomas, osteosarcomas, and small cell lung cancers.

In one embodiment polynucleotides encoding Ch14.18 fragments or variants thereof may be used in the treatment of neuroblastoma. In one embodiment, polynucleotides encoding Ch14.18 fragments or variants thereof may be used in the treatment of neuroblastoma alone or in combination with other standard treatments, including but not limited to combination chemotherapy, surgery, stem cell rescue, radiation therapy, including but not limited to targetd MIGB therapy, isotretinoin (cis-retinoic acid), granulocyte-macrophage colony-stimulating factor (GM-CSF), and interleukin-2(IL-2), lenalidomide, REVLIMID®.

In one embodiment, polynucleotides encoding Ch14.18 fragments or variants thereof may encode ch14.18 further comprising an IL2 sequence, an immunocytokine (IC) formed by linking IL-2 to the carboxyl end of the constant region of ch14.18 mAb (as described in Gillies et al, 1992, the content of which is herein incorporated by reference in its entirety) or a bispecific antibody that detects 11-2, administered in combination with 11-2.

### Racotumomab Parent Molecule or Antibody

In one embodiment, the polynucleotides of the present invention may encode Racotumomab, fragments or variants thereof.

Racotumomab, also known as VAXIRA®, 1E10, and ECACC Deposit No. 97112901, is an anti-idiotypic mouse IgG1kappa monoclonal antibody that mimics the three dimensional structure of NGc gangliosides. P3 MoAb, the murine anti-NGc monoclonal antibody that was used as an immunogen to create racotumomab, is produced by the hybridoma deposited under the accession number ECACC 94113026. The development of racotumomab, deposited under ECACC Deposit No. 97112901, is described in U.S. Pat. No. 6,491,914, and in Alfonso et al., 2002, the contents of each of which are herein incorporated by reference in their entirety. Racotumomab is under development by Recombio, an international public-private consortium with the participation of the Center of Molecular Immunology at Havana, Cuba (CIM) and researchers from Buenos Aires University and National University of Quilmes in Argentina.

Racotumomab triggers an immune response against the tumor antigen N-glycolyl (NGc) ganglioside, a type of glycolipid present in the cell membrane on the cell surface of in tumors, including lung, breast, melanoma, and neuroectodermal pediatric tumors, such as neuroblastoma.

Certain sequences encoding the polynucleotide fragments, domains or heavy or light chains for Racotumomab are given in Table 71. The table is not an exhaustive list and any fragment or portion of the sequence which may be encoded in the polynucleotides of the invention.

TABLE 71
Table of Racotumomab Sequences

| Description | Sequence | SEQ ID NO. |
|---|---|---|
| Light chain | DIQMTQTTSSLSASLGDRVTISCRASQDISNYLNWYQQ | 357 |
| | KPDGTVKLLIYYTSRLHSGVPSRFSGSGSGTDYSLTISN | |
| | LEQEDIATYFCQQGNTLPWTFGGGTKLEIKRADAAPTV | |
| | SIFPPSSEQLTSGGASVVCFLNNFYPKDINVKWKIDGSE | |
| | RQNGVLNSWTDQDSKDSTYSMSSTLTLTKDEYERHNS | |
| | YTCEATHKTSTSPIVKSFNRNEC | |
| Heavy Chain | QVQLQQSGAELVKPGASVKLSCKASGYTFTSYDINWV | 358 |
| | RQRPEQGLEWIGWIFPGDGSTKYNEKFKGKATLTTDK | |
| | SSSTAYMQLSRLTSEDSAVYFCAREDYYDNSYYFDYW | |
| | GQGTTLTVSSAKTTPPSVYPLAPGSAAQTNSMVTLGCL | |
| | VKGYFPEPVTVTWNSGSLSSGVHTFPAVLQSDLYTLSS | |
| | SVTVPSSPRPSETVTCNVAHPASSTKVDKKIVPRDCGC | |
| | KPCICTVPEVSSVFIFPPKPKDVLTITLTPKVTCVVVDIS | |
| | KDDPEVQFSWFVDDVEVHTAQTQPREEQFNSTFRSVS | |
| | ELPIMHQDWLNGKEFKCRVNSAAFPAPIEKTISKTKGR | |
| | PKAPQVYTIPPPKEQMAKDKVSLTCMITDFFPEDITVE | |
| | WQWNGQPAENYKNTQPIMNTNGSYFVYSKLNVQKSN | |
| | WEAGNTFTCSVLHEGLHNHHTEKSLSHSPGK | |

In one embodiment, polynucleotides encoding Racotumomab, fragments or variants thereof may be used to treat and/or prevent small cell lung cancer (SCLC).

In one embodiment, polynucleotides encoding Racotumomab, fragments or variants thereof may be used to treat and/or prevent non-small cell lung cancer (NSCLC).

In one embodiment, polynucleotides encoding Racotumomab, fragments or variants thereof may be used in the treatment of cancers expressing NGc-GM3 gangliosides, including, but not limited to NSCLC, SCLC, melanoma, breast cancer, and tumors of neuroectodermal origin. Non-limiting examples of tumors of neuroectodermal origin include neuroblastoma, Ewing's sarcoma, Wilm's tumor and retinoblastoma.

In one embodiment, polynucleotides encoding Racotumomab, fragments or variants thereof may be used in the treatment of cancers expressing NGc-GM3 gangliosides in combination with another standard care regimen, such as chemotherapeutic agents, a radiation therapy, targeted therapy, immunotherapy, or surgery.

### Dupilumab Parent Molecule or Antibody

In one embodiment, the polynucleotides of the present invention may encode Dupilumab, fragments or variants thereof.

Dupilumab, also known as REGN668 and SAR231893, is a fully-human IgG4k monoclonal antibody that is being developed by Regeneron and Sanofi. Dupilumab is a fully human investigational monoclonal antibody delivered by subcutaneous injection that targets the alpha subunit of the interleukin 4 receptor (IL-4R alpha). By blocking IL-4R alpha, Dupilumab modulates signaling of both IL-4 and IL-13, drivers of Th2 (Type 2 helper T cell) immune response.

Dupilumab was generated using VelocImmune™ technology. The technique employs a transgenic mouse, in which the endogenous immunoglobulin heavy and light chain variable regions are replaced with the corresponding human variable regions. After the mouse is challenged with the antigen of interest, DNA encoding the variable regions of the heavy and light chains of the antibody are isolated and operably linked to DNA encoding the human heavy and light chain constant regions. The DNA is then expressed in a cell capable of expressing the fully human antibody.

Sequences of Dupilumab are disclosed in U.S. Pat. No. 8,075,887 and International Patent publication WO2010053751, the contents of each of which are herein incorporated by reference in their entirety. Polynucleotides of the invention may encode the antibodies or fragments thereof which bind to IL-4R alpha such as, but not limited to, SEQ ID NO 1 in U.S. Pat. No. 8,338,135 or SEQ ID NO:274 of U.S. Pat. No. 8,075,887, the contents of which is herein incorporated by reference in its entirety.

In one embodiment, the polynucleotides described herein may encode at least one Dupilumab sequence, fragment or variant thereof disclosed in U.S. Pat. Nos. 8,338,135 and 8,075,887, the contents of each of which are herein incorporated by reference in their entirety.

Certain sequences encoding the polynucleotide fragments, domains or heavy or light chains for Dupilumab are given in Table 72. The table is not an exhaustive list and any fragment or portion of the sequence which may be encoded in the polynucleotides of the invention.

TABLE 72
Table of Dupilumab Sequences

| | | | SEQ |
|---|---|---|---|
| | | | ID |
| Description | Sequence | Source | NO. |
| Heavy chain | EVQLVESGGGLEQPGGSLRLSCAGSGF | | 359 |
| | TFRDYAMTWVRQAPGKGLEWVSSISGS | | |
| | GGNTYYADSVKGRFTISRDNSKNTLYL | | |
| | QMNSLRAEDTAVYYCAKDRLSITIRPR | | |
| | YYGLDVWGQGTTVTVSSASTKGPSVFP | | |
| | LAPCSRSTSESTAALGCLVKDYFPEPVT | | |
| | VSWNSGALTSGVHTFPAVLQSSGLYSL | | |
| | SSVVTVPSSSLGTKTYTCNVDHKPSNTK | | |
| | VDKRVESKYGPPCPPCPAPEFLGGPSVF | | |
| | LFPPKPKDTLMISRTPEVTCVVVDVSQE | | |
| | DPEVQFNWYVDGVEVHNAKTKPREEQ | | |
| | FNSTYRVVSVLTVLHQDWLNGKEYKC | | |
| | KVSNKGLPSSIEKTISKAKGQPREPQVY | | |
| | TLPPSQEEMTKNQVSLTCLVKGFYPSDI | | |
| | AVEWESNGQPENNYKTTPPVLDSDGSF | | |
| | FLYSRLTVDKSRWQEGNVFSCSVMHEA | | |
| | LHNHYTQKSLSLSLG | | |
| Heavy chain | EVQLVESGGGLEQPGGSLRLSCAGSGF | From U.S. Pat. | 360 |
| variable region | TFRDYAMTWVRQAPGKGLEWVSSISGS | No. 8,075,887 | |
| | GGNTYYADSVKGRFTISRDNSKNTLYL | SEQ ID NO: 162 | |
| | QMNSLRAEDTAVYYCAKDRLSITIRPR | | |
| | YYGLDVWGQGTTVTVS | | |
| Heavy chain | GFTFRDYA | From U.S. Pat. | 361 |
| CDR1 | | No. 8,075,887 | |
| | | SEQ ID NO: 148 | |
| Heavy chain | ISGSGGNT | From U.S. Pat. | 362 |
| CDR2 | | No. 8,075,887 | |
| | | From U.S. Pat. | |
| | | No. 8,075,887 | |
| | | SEQ ID NO: 150 | |
| Heavy chain | AKDRLSITIRPRYYGLDV | From U.S. Pat. | 363 |
| CDR3 | | No. 8,075,887 | |
| | | SEQ ID NO: 152 | |
| Light chain | DIVMTQSPLSLPVTPGEPASISCRSSQSL | 364 | |
| | LYSIGYNYLDWYLQKSGQSPQLLIYLGS | | |
| | NRASGVPDRFSGSGSGTDFTLKISRVEA | | |
| | EDVGFYYCMQALQTPYTFGQGTKLEIK | | |
| | RTVAAPSVFIFPPSDEQLKSGTASVVCL | | |
| | LNNFYPREAKVQWKVDNALQSGNSQE | | |
| | SVTEQDSKDSTYSLSSTLTLSKADYEKH | | |
| | KVYACEVTHQGLSSPVTKSFNRGEC | | |
| Light chain | DIVMTQSPLSLPVTPGEPASISCRSSQSL | From U.S. Pat. | 365 |
| variable region | LYSIGYNYLDWYLQKSGQSPQLLIYLGS | No. 8,075,887 | |
| | NRASGVPDRFSGSGSGTDFTLKISRVEA | SEQ ID NO: 164 | |
| | EDVGFYYCMQALQTPYTFGQGTKLEIK | | |
| Light chain | QSLLYSIGYNY | From U.S. Pat. | 366 |
| CDR1 | | No. 8,075,887 | |
| | | SEQ ID NO: 156 | |
| Light chain | LGS | From U.S. Pat. | — |
| CDR2 | | No. 8,075,887 | |
| | | SEQ ID NO: 158 | |
| Light chain | MQALQTPYT | From U.S. Pat. | 367 |
| CDR3 | | No. 8,075,887 | |
| | | SEQ ID NO: 160 | |

In one embodiment, polynucleotides encoding Dupilumab, fragments or variants thereof may be used to treat and/or prevent moderate-to-severe asthma.

In one embodiment, polynucleotides encoding Dupilumab, fragments or variants thereof may be used to treat and/or prevent diseases or disorders associated with overactive Th2-pathway activity and/or in elevated eosinophil levels.

In one embodiment, polynucleotides encoding Dupilumab, fragments or variants thereof may be used to treat diseases or disorders which can be treated by binding to the interleukin-4 receptor (IL-4R). In one embodiment, polynucleotides encoding Dupilumab, fragments or variants thereof may be used to prevent or treat conditions, characterized by abnormal or excess expression of IL-4, or by an abnormal host response to IL-4 production, including asthma, atopic dermatitis, rhinosinusitis w/eosinophilic polyps, atopic dermatitis, food allergy, eosinophilic digestive disorders and hypereosinophilic syndromes. Further non-limiting examples are arthritis (including septic arthritis), herpetiformis, chronic idiopathic urticaria, scleroderma, hypertrophic scarring, Whipple's Disease, benign prostate hyperplasia, pulmonary disorders such as asthma (mild, moderate or severe), inflammatory disorders such as inflammatory bowel disease, allergic reactions, Kawasaki disease, sickle cell disease, Churg-Strauss syndrome, Grave's disease, pre-eclampsia, Sjogren's syndrome, autoimmune lympho proliferative syndrome, autoimmune hemolytic anemia, Barrett's esophagus, autoimmune uveitis, tuberculosis, atopic dermatitis, ulcerative colitis, fibrosis, and nephrosis (as described in U.S. Pat. No. 7,186,809, the contents of which are herein incorporated by reference in their entirety).

In one embodiment, polynucleotides encoding Dupilumab, fragments or variants thereof may be used to treat and/or prevent atopic dermatitis (AD). Atopic dermatitis (AD) is a chronically relapsing inflammatory disease, more common in infants and children than in adults and very rare after midlife, and is characterized by itching eczematous lesions. Atopy is tendency to produce immunoglobulin E (IgE) antibodies in response to low doses of allergens and to develop typical symptoms such as asthma, rhinoconjunctivitis, and eczema or dermatitis.

In one embodiment, polynucleotides encoding Dupilumab, fragments or variants thereof may be used to treat and/or prevent rhinosinusitis w/eosinophilic polyps, food allergy, eosinophilic digestive disorders and hypereosinophilic syndromes.

### Clivatiuzumab Tetraxetan Parent Molecule or Antibody

In one embodiment, the polynucleotides of the present invention may encode Clivatuzumab tetraxetan, fragments or variants thereof.

Clivatuzumab tetraxetan, also known as 90Y-hPAM4, is a humanized monoclonal antibody that targets a mucin antigen found on pancreatic cancer cells, but not pancreatitis, normal pancreas or most other normal tissues (Gold et al., 2013), developed by Immunomedics. Clivatuzumab tetraxetan is conjugated to a linker that facilitates complexing the antibody with radiometals and is radiolabeled with yttrium-90, which is delivered directly to the tumor.

Clivatuzumab tetraxetan and the parent antibody clivatuzumab are humanized IgG1k monoclonal antibodies. The molecular weight of clivatuzumab is 145.7 kDa. Clivatuzumab is the humanized form of PAM4 murine monoclonal antibody (Gold et al., 1994 and U.S. Pat. No. 7,282,567, also described in U.S. Pat. Nos. 8,435,529 and 8,586,050, the contents of each of which are herein incorporated by reference in their entirety). The three complementary determining regions (CDR1, CDR 2 and CDR3) of humanized light chain have amino acid sequences are derived from the corresponding CDRs of the mouse immunoglobulin light chain variable regions and a variable region framework from a human kappa light chain variable framework sequence. Similarly, the three complementary determining regions (CDR1, CDR 2 and CDR3) of humanized heavy chain have amino acid sequences from the corresponding CDRs of the mouse immunoglobulin heavy chain variable regions and a variable region framework from a human heavy chain variable framework sequence. In clivatuzumab tetraxetan the parent antibody clivatuzumab is conjugated to a linker that facilitates complexing with radiometals. The linker, tetraxetan (also known as DOTA), functions as a chelator for yttrium-90.

Clivatuzumab tetraxetan contains a humanized, highly specific antibody that targets an antigen PAM4 on mucin MUC1 found on pancreatic cancer cells. MUC1 has been found by tissue staining to be present on about 85% of pancreatic cancers but is not found on normal pancreas or tissue from patients with pancreatitis (Gold et al., 2013). When the antibody-linker complex is radiolabeled with yttrium-90, this enables delivery of high intensity, deep penetrating radiation directly to the pancreatic tumor cells. In preclinical models, pancreatic cancer has been responsive to radioimmunotherapy with radiolabeled clivatuzumab tetraxetan (Cardillo et al, 2001). Preclinical studies have also found further improvements when ⁹⁰Y-clivatuzumab tetraxetan was combined with gemcitabine, a nucleoside analog that inhibits nucleic acid synthesis and an approved therapy for pancreatic cancer and a known radiosensitizer (Morgan et al., 2008).

Polynucleotides of the present invention may encode Clivatuzumab tetraxetan fragments or variants thereof and other anti-PAM4 antibodies described in U.S. Pat. Nos. 7,282,567, 8,435,529 and 8,586,050, the contents of each of which are herein incorporated by reference in their entirety.

In one embodiment, the polynucleotides described herein may encode at least one Clivatuzumab tetraxetan sequence, fragment or variant thereof disclosed in U.S. Pat. No. 8, U.S. Pat. Nos. 7,282,567, 8,435,529 and 8,586,050, the contents of each of which are herein incorporated by reference in their entirety.

Certain sequences encoding the polynucleotide fragments, domains or heavy or light chains for Clivatuzumab tetraxetan are given in Table 73. The table is not an exhaustive list and any fragment or portion of the sequence which may be encoded in the polynucleotides of the invention.

TABLE 73
Table of Clivatuzumab tetraxetan Sequences

| | | | SEQ ID |
|---|---|---|---|
| Description | Sequence | Source | NO. |
| Light chain | DIQLTQSPSSLSASVGDRVTMTCSASSS | | 368 |
| | VSSSYLYWYQQKPGKAPKLWIYSTSNL | | |
| | ASGVPARFSGSGSGTDFTLTISSLQPEDS | | |
| | ASYFCHQWNRYPYTFGGGTRLEIKRTV | | |
| | AAPSVFIFPPSDEQLKSGTASVVCLLNN | | |
| | FYPREAKVQWKVDNALQSGNSQESVT | | |
| | EQDSKDSTYSLSSTLTLSKADYEKHKV | | |
| | YACEVTHQGLSSPVTKSFNRGEC | | |
| Heavy Chain | QVQLQQSGAEVKKPGASVKVSCEASG | | 369 |
| | YTFPSYVLHWVKQAPGQGLEWIGYINP | | |
| | YNDGTQYNEKFKGKATLTRDTSINTAY | | |
| | MELSRLRSDDTAVYYCARGFGGSYGFA | | |
| | YWGQGTLVTVSSASTKGPSVFPLAPSSK | | |
| | STSGGTAALGCLVKDYFPEPVTVSWNS | | |
| | GALTSGVHTFPAVLQSSGLYSLSSVVTV | | |
| | PSSSLGTQTYICNVNHKPSNTKVDKRVE | | |
| | PKSCDKTHTCPPCPAPELLGGPSVFLFPP | | |
| | KPKDTLMISRTPEVTCVVVDVSHEDPE | | |
| | VKFNWYVDGVEVHNAKTKPREEQYNS | | |
| | TYRVVSVLTVLHQDWLNGKEYKCKVS | | |
| | NKALPAPIEKTISKAKGQPREPQVYTLP | | |
| | PSREEMTKNQVSLTCLVKGFYPSDIAVE | | |
| | WESNGQPENNYKTTPPVLDSDGSFFLY | | |
| | SKLTVDKSRWQQGNVFSCSVMHEALH | | |
| | NHYTQKSLSLSPGK | | |
| Light chain | SASSSVSSSYLY | From U.S. Pat. | 370 |
| CDR1 | | No. 7,282,567, | |
| | | 8,435,529 and | |
| | | 8,586,050 | |
| | | SEQ ID NO: 1 | |
| Light chain | STSNLAS | From U.S. Pat. | 371 |
| CDR2 | | No. 7,282,567, | |
| | | 8,435,529 and | |
| | | 8,586,050 | |
| | | SEQ ID NO: 2 | |
| Light chain | HQWNRYPYT | From U.S. Pat. | 372 |
| CDR3 | | No. 7,282,567, | |
| | | 8,435,529 and | |
| | | 8,586,050 | |
| | | SEQ ID NO: 3 | |
| Heavy chain | SYVLH | From U.S. Pat. | 373 |
| CDR1 | | No. 7,282,567, | |
| | | 8,435,529 and | |
| | | 8,586,050 | |
| | | SEQ ID NO: 4 | |
| Heavy chain | YINPYNDGTQYNEKFKG | From U.S. Pat. | 374 |
| CDR2 | | No. 7,282,567, | |
| | | 8,435,529 and | |
| | | 8,586,050 | |
| | | SEQ ID NO: 5 | |
| Heavy chain | GFGGSYGFAY | From U.S. Pat. | 375 |
| CDR3 | | No. 7,282,567, | |
| | | 8,435,529 and | |
| | | 8,586,050 | |
| | | SEQ ID NO: 6 | |

In one embodiment, polynucleotides encoding Clivatuzumab tetraxetan, fragments or variants thereof may be used to treat and/or prevent pancreatic cancer.

In one embodiment, polynucleotides encoding Clivatuzumab tetraxetan, fragments or variants thereof may be used for imaging of pancreatic cancer. In one embodiment, the polynucleotides encoding Clivatuzumab tetraxetan, fragments or variants thereof may be used in combination with additional chemotherapeutic agents and/or radionuclides.

According to the present invention, an intrabody construct is a polynucleotide which has been modified for expression inside a target cell and where the expression product binds an intracellular protein. Such constructs may have sub picomolar binding affinities and may be formulated for targeting to particular sites or tissues. For example, intrabody constructs may be formulated in any of the lipid nanoparticle formulations disclosed herein.

Bicistronic and/or Pseudo-Bicistronic Constructs

According to the present invention, a bicistronic construct is a polynucleotide encoding a two-protein chain antibody on a single polynucleotide strand. (FIG. 2B) A pseudo-bicistronic construct is a polynucleotide encoding a single chain antibody discontinuously on a single polynucleotide strand. For bicistronic constructs, the encoded two strands or two portions/regions and/or domains (as is the case with pseudo-bicistronic) are separated by at least one nucleotide not encoding the strands or domains. More often the separation comprises a cleavage signal or site or a non-coding region of nucleotides. Such cleavage sites include, for example, furin cleavage sites encoded as an “RKR” site in the resultant polypeptide.

### Single Domain Constructs

According to the present invention, a single domain construct comprises one or two polynucleotides encoding a single monomeric variable antibody domain. See FIGS. 3B and 4B for examples. Typically single domain antibodies comprise one variable domain (VH) of a heavy-chain antibody.

### Single Chain Fv Constructs

According to the present invention, a single chain Fv constructs is a polynucleotide encoding at least two coding regions and a linker region. The scFv construct may encode a fusion protein of the variable regions of the heavy (VH) and light chains (VL) of immunoglobulins, connected with a short linker peptide of ten to about 25 amino acids. See FIG. 3A for an example. The linker is usually rich in glycine for flexibility, as well as serine or threonine for solubility, and can either connect the N-terminus of the VH with the C-terminus of the VL, or vice versa. Other linkers include those known in the art and disclosed herein.

### Bispecific Constructs

According to the present invention, a bispecific construct is a polynucleotide encoding portions or regions of two different antibodies. Bispecific constructs encode polypeptides which may bind two different antigens. See FIG. 4A for an example. Polynucleotides of the present invention may also encode trispecific antibodies having an affinity for three antigens.

### Linkers

Examples of linkers which may be used in the polynucleotides of the present invention include those in Table 74.

TABLE 74
Linkers

| Name | Sequence in polynucleotide | SEQ ID NO |
|---|---|---|
| PLrigid | GAAGCTGCTGCAAGAGAAGCTGCAGCTAGG | 376 |
| PLrigid is a 20 a.a. peptide that is based | GAGGCTGCAGCTAGGGAGGCTGCTGCAAGA | |
| on an alpha-helix motif (EAAAR) (SEQ | | |
| ID NO: 388) (Merutka et al., 1991; | | |
| Sommese et al., 2010) | | |
| 2aa GS linker | GGCAGC | — |
| Highly flexibly glycine linker | | |
| 6aa [GS]x linker | GGTAGCGGCAGCGGTAGC | 377 |
| Highly flexible 6 amino acid linker. | | |
| Translates to gsgsgs (SEQ ID NO: 389). | | |
| Codon-optimized for E. coli, yeast, | | |
| mammalian | | |
| 10 aa flexible protein domain linker | GGTGAAAATTTGTATTTTCAATCTGGTGGT | 378 |
| 8 aa protein domain linker | TCCGCTTGTTACTGTGAGCTTTCC | 379 |
| 15 aa flexible glycine-serine protein | GGTGGAGGAGGTTCTGGAGGCGGTGGAAGT | 380 |
| domain linker; Freiburg standard | GGTGGCGGAGGTAGC | |
| Short Linker (Gly-Gly-Ser-Gly) (SEQ ID | GGTGGTTCTGGT | 381 |
| NO: 390) | | |
| Middle Linker (Gly-Gly-Ser-Gly)x2 (SEQ | GGTGGTTCTGGTGGTGGTTCTGGT | 382 |
| ID NO: 391) | | |
| Long Linker (Gly-Gly-Ser-Gly)x3 (SEQ ID | GGTGGTTCTGGTGGTGGTTCTGGTGGTGGTT | 383 |
| NO: 392) | CTGGT | |
| GSAT Linker | GGTGGTTCTGCCGGTGGCTCCGGTTCTGGCT | 384 |
| | CCAGCGGTGGCAGCTCTGGTGCGTCCGGCAC | |
| | GGGTACTGCGGGTGGCACTGGCAGCGGTTCC | |
| | GGTACTGGCTCTGGC | |
| SEG-Linker | GGTGGTTCTGGCGGCGGTTCTGAAGGTGGCG | 385 |
| | GCTCCGAAGGCGGCGGCAGCGAGGGCGGTG | |
| | GTAGCGAAGGTGGTGGCTCCGAGGGTGGCG | |
| | GTTCCGGCGGCGGTAGC | |

Table references: Merutka G, Shalongo W, Stellwagen E. (1991) A model peptide with enhanced helicity. Biochem. 30: 4245-4248 and Sommese R F, Sivaramakrishnan S, Baldwin R L, Spudich J A. (2010) Helicity of short E-R/K peptides. Protein Sci. 19: 2001-2005.

In one embodiment, the length of a region encoding at least one peptide polypeptide of interest of the polynucleotides present invention is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides). As used herein, such a region may be referred to as a “coding region” or “region encoding.”

In one embodiment, the polynucleotides of the present invention is or functions as a messenger RNA (mRNA). As used herein, the term “messenger RNA” (mRNA) refers to any polynucleotide which encodes at least one peptide or polypeptide of interest and which is capable of being translated to produce the encoded peptide polypeptide of interest in vitro, in vivo, in situ or ex vivo.

In one embodiment, the polynucleotides of the present invention may be structurally modified or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In one embodiment, the polynucleotides of the present invention, such as IVT polynucleotides or circular polynucleotides, may have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine. In another embodiment, the polynucleotides may have a uniform chemical modification of two, three, or four of the same nucleoside type throughout the entire polynucleotide (such as all uridines and all cytosines, etc. are modified in the same way).

When the polynucleotides of the present invention are chemically and/or structurally modified the polynucleotides may be referred to as “modified polynucleotides.”

In one embodiment, the polynucleotides of the present invention may include a sequence encoding a self-cleaving peptide. The self-cleaving peptide may be, but is not limited to, a 2A peptide. As a non-limiting example, the 2A peptide may have the protein sequence: GSGATNFSLLKQAGDVEENPGP (SEQ ID NO: 386), fragments or variants thereof. In one embodiment, the 2A peptide cleaves between the last glycine and last proline. As another non-limiting example, the polynucleotides of the present invention may include a polynucleotide sequence encoding the 2A peptide having the protein sequence GSGATNFSLLKQAGDVEENPGP (SEQ ID NO: 386) fragments or variants thereof.

One such polynucleotide sequence encoding the 2A peptide is GGAAGCGGAGCTACTAACTTCAGCCTGCTGAAGCAGGCTGGAGACGTGGAG GAGAACCCTGGACCT (SEQ ID NO: 387). The polynucleotide sequence of the 2A peptide may be modified or codon optimized by the methods described herein and/or are known in the art.

In one embodiment, this sequence may be used to separate the coding region of two or more polypeptides of interest. As a non-limiting example, the sequence encoding the 2A peptide may be between a first coding region A and a second coding region B (A-2Apep-B). The presence of the 2A peptide would result in the cleavage of one long protein into protein A, protein B and the 2A peptide. Protein A and protein B may be the same or different peptides or polypeptides of interest.

In another embodiment, the 2A peptide may be used in the polynucleotides of the present invention to produce two, three, four, five, six, seven, eight, nine, ten or more proteins.

### IVT Polynucleotide Architecture

Traditionally, the basic components of an mRNA molecule include at least a coding region, a 5′UTR, a 3′UTR, a 5′ cap and a poly-A tail. The IVT polynucleotides of the present invention may function as mRNA but are distinguished from wild-type mRNA in their functional and/or structural design features which serve to overcome existing problems of effective polypeptide production using nucleic-acid based therapeutics.

IVT constructs and their features may be those of the primary constructs as described in copending International Publication No. WO2013151666, filed Mar. 9, 2013 (Attorney Docket No. M300) and International Application No. PCT/US2014/069155 (Attorney Docket No. M073), the contents of which are incorporated by reference in their entirety.

### Chimeric Polynucleotide Architecture

Chimeric polynucleotides or RNA constructs of the present invention maintain a modular organization similar to IVT polynucleotides, but the chimeric polynucleotides comprise one or more structural and/or chemical modifications or alterations which impart useful properties to the polynucleotide. As such, the chimeric polynucleotides which are modified mRNA molecules of the present invention are termed “chimeric modified mRNA” or “chimeric mRNA.”

It is to be understood that the polynucleotides of the present invention may be encoded by a chimeric polynucleotide, RNA construct, chimeric modified mRNA or chimeric mRNA. Chimeric polynucleotides, formulations and compositions comprising chimeric polynucleotides, and methods of making, using and administering chimeric polynucleotides are also described in co-pending International Application No. PCT/US2014/053907, filed Sep. 3, 2014 (Attorney Docket Number M057) the contents of which is incorporated by reference in its entirety.

### Circular Polynucleotide Architecture

The present invention contemplates chimeric polynucleotides which are circular or cyclic. As the name implies circular polynucleotides are circular in nature meaning that the termini are joined in some fashion, whether by ligation, covalent bond, common association with the same protein or other molecule or complex or by hybridization. Any of the cicular polynucleotides as taught in, for example, International Application No. PCT/2014/053904, filed Sep. 3, 2014 (Attorney docket number M51), the contents of each of which are incorporated herein by reference in their entirety, may be made chimeric according to the present invention.

### Multimers of Polynucleotides

According to the present invention, multiple distinct chimeric polynucleotides and/or IVT polynucleotides may be linked together through the 3′-end using nucleotides which are modified at the 3′-terminus. Chemical conjugation may be used to control the stoichiometry of delivery into cells. For example, the glyoxylate cycle enzymes, isocitrate lyase and malate synthase, may be supplied into cells at a 1:1 ratio to alter cellular fatty acid metabolism. This ratio may be controlled by chemically linking chimeric polynucleotides and/or IVT polynucleotides using a 3′-azido terminated nucleotide on one polynucleotides species and a C5-ethynyl or alkynyl-containing nucleotide on the opposite polynucleotide species. The modified nucleotide is added post-transcriptionally using terminal transferase (New England Biolabs, Ipswich, Mass.) according to the manufacturer's protocol. After the addition of the 3′-modified nucleotide, the two polynucleotides species may be combined in an aqueous solution, in the presence or absence of copper, to form a new covalent linkage via a click chemistry mechanism as described in the literature.

In another example, more than two chimeric polynucleotides and/or IVT polynucleotides may be linked together using a functionalized linker molecule. For example, a functionalized saccharide molecule may be chemically modified to contain multiple chemical reactive groups (SH—, NH₂—, N₃, etc. . . . ) to react with the cognate moiety on a 3′-functionalized mRNA molecule (i.e., a 3′-maleimide ester, 3′-NHS-ester, alkynyl). The number of reactive groups on the modified saccharide can be controlled in a stoichiometric fashion to directly control the stoichiometric ratio of conjugated chimeric polynucleotides and/or IVT polynucleotides.

In one embodiment, the chimeric polynucleotides and/or IVT polynucleotides may be linked together in a pattern. The pattern may be a simple alternating pattern such as CD[CD]ₓ where each “C” and each “D” represent a chimeric polynucleotide, IVT polynucleotide, different chimeric polynucleotides or different IVT polynucleotides. The pattern may repeat x number of times, where x=1-300. Paterns may also be alternating multiples such as CCDD[CCDD]ₓ (an alternating double multiple) or CCCDDD[CCCDDD]ₓ (an alternating triple multiple) pattern. The alternating double multiple or alternating triple multiple may repeat x number of times, where x=1-300.

### Conjugates and Combinations of Polynucleotides

In order to further enhance protein production, polynucleotides of the present invention can be designed to be conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug.

Conjugation may result in increased stability and/or half life and may be particularly useful in targeting the polynucleotides to specific sites in the cell, tissue or organism.

According to the present invention, the polynucleotides may be administered with, conjugated to or further encode one or more of RNAi agents, siRNAs, shRNAs, miRNAs, miRNA binding sites, antisense RNAs, ribozymes, catalytic DNA, tRNA, RNAs that induce triple helix formation, aptamers or vectors, and the like.

### Bifunctional Polynucleotides

In one embodiment of the invention, antibody compositions may comprise bifunctional polynucleotides (e.g., bifunctional IVT polynucleotides, bifunctional chimeric polynucleotides or bifunctional circular polynucleotides). As the name implies, bifunctional polynucleotides are those having or capable of at least two functions. These molecules may also by convention be referred to as multi-functional.

The multiple functionalities of bifunctional polynucleotides may be encoded by the RNA (the function may not manifest until the encoded product is translated) or may be a property of the polynucleotide itself. It may be structural or chemical. Bifunctional modified polynucleotides may comprise a function that is covalently or electrostatically associated with the polynucleotides. Further, the two functions may be provided in the context of a complex of a chimeric polynucleotide and another molecule.

### Noncoding Polynucleotides

As described herein, provided are polynucleotides having sequences that are partially or substantially not translatable, e.g., having a noncoding region. As one non-limiting example, the noncoding region may be the first region of the IVT polynucleotide or the circular polynucleotide. Alternatively, the noncoding region may be a region other than the first region. As another non-limiting example, the noncoding region may be the A, B and/or C region of the chimeric polynucleotide.

Such molecules are generally not translated, but can exert an effect on the immune response or protein production by one or more of binding to and sequestering one or more translational machinery components such as a ribosomal protein or a transfer RNA (tRNA), thereby effectively reducing protein expression in the cell or modulating one or more pathways or cascades in a cell which in turn alters protein levels. The polynucleotide may contain or encode one or more long noncoding RNA (lncRNA, or lincRNA) or portion thereof, a small nucleolar RNA (sno-RNA), micro RNA (miRNA), small interfering RNA (siRNA) or Piwi-interacting RNA (piRNA). Examples of such lncRNA molecules and RNAi constructs designed to target such lncRNA any of which may be encoded in the polynucleotides are taught in International Publication, WO2012/018881 A2, the contents of which are incorporated herein by reference in their entirety.

### Polypeptides of Interest

According to the present invention, the polynucleotide may be designed to encode one or more polypeptides of interest or fragments thereof. Such polypeptide of interest may include, but is not limited to, whole polypeptides, a plurality of polypeptides or fragments of polypeptides, which independently may be encoded by one or more regions or parts or the whole of a polynucleotide. As used herein, the term “polypeptides of interest” refer to any polypeptide which is selected to be encoded within, or whose function is affected by, the polynucleotides of the present invention.

As used herein, “polypeptide” means a polymer of amino acid residues (natural or unnatural) linked together most often by peptide bonds. The term, as used herein, refers to proteins, polypeptides, and peptides of any size, structure, or function. In one embodiment, the polypeptides of interest are antibodies encoded by the polynucleotides as described herein.

In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides such as antibodies or insulin and may be associated or linked. Most commonly disulfide linkages are found in multichain polypeptides. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to polypeptide sequences means the corresponding sequence of other species having substantial identity to a second sequence of a second species.

“Analogs” is meant to include polypeptide variants which differ by one or more amino acid alterations, e.g., substitutions, additions or deletions of amino acid residues that still maintain one or more of the properties of the parent or starting polypeptide.

The present invention contemplates several types of compositions which are polypeptide based including variants and derivatives. These include substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this invention. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to polypeptides are those with one or more amino acids in the native or starting amino acid sequence removed. Ordinarily, deletional variants will have one or more amino acids deleted in a particular region of the molecule.

“Covalent derivatives” when referring to polypeptides include modifications of a native or starting protein with an organic proteinaceous or non-proteinaceous derivatizing agent, and/or post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the polypeptides produced in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to polypeptides are defined as distinct amino acid sequence-based components of a molecule. Features of the polypeptides encoded by the polynucleotides of the present invention include surface manifestations, local conformational shape, folds, loops, half-loops, domains, half-domains, sites, termini or any combination thereof.

As used herein when referring to polypeptides the term “surface manifestation” refers to a polypeptide based component of a protein appearing on an outermost surface.

As used herein when referring to polypeptides the term “local conformational shape” means a polypeptide based structural manifestation of a protein which is located within a definable space of the protein.

As used herein when referring to polypeptides the term “fold” refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to polypeptides the term “loop” refers to a structural feature of a polypeptide which may serve to reverse the direction of the backbone of a peptide or polypeptide. Where the loop is found in a polypeptide and only alters the direction of the backbone, it may comprise four or more amino acid residues. Oliva et al. have identified at least 5 classes of protein loops (J. Mol Biol 266 (4): 814-830; 1997). Loops may be open or closed. Closed loops or “cyclic” loops may comprise 2, 3, 4, 5, 6, 7, 8, 9, 10 or more amino acids between the bridging moieties. Such bridging moieties may comprise a cysteine-cysteine bridge (Cys-Cys) typical in polypeptides having disulfide bridges or alternatively bridging moieties may be non-protein based such as the dibromozylyl agents used herein.

As used herein when referring to polypeptides the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the term “half-domain” means a portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that subdomains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to polypeptides the terms “site” as it pertains to amino acid based embodiments is used synonymously with “amino acid residue” and “amino acid side chain.” A site represents a position within a peptide or polypeptide that may be modified, manipulated, altered, derivatized or varied within the polypeptide based molecules of the present invention.

As used herein the terms “termini” or “terminus” when referring to polypeptides refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a desired component of a polypeptide to be encoded by the polynucleotide of the invention, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as, but not limited to, site directed mutagenesis or a priori incorporation during chemical synthesis. The resulting modified molecules may then be tested for activity using in vitro or in vivo assays such as those described herein or any other suitable screening assay known in the art.

According to the present invention, the polypeptides may comprise a consensus sequence which is discovered through rounds of experimentation. As used herein a “consensus” sequence is a single sequence which represents a collective population of sequences allowing for variability at one or more sites.

As recognized by those skilled in the art, protein fragments, functional protein domains, and homologous proteins are also considered to be within the scope of polypeptides of interest of this invention. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the invention. In certain embodiments, a polypeptide to be utilized in accordance with the invention includes 2, 3, 4, 5, 6, 7, 8, 9, 10, or more mutations as shown in any of the sequences provided or referenced herein.

In one embodiment, polynucleotides may encode variant polypeptides which have a certain identity with a reference polypeptide sequence. As used herein, a “reference polypeptide sequence” refers to a starting polypeptide sequence. Reference sequences may be wild type sequences or any sequence to which reference is made in the design of another sequence. A “reference polypeptide sequence” may, e.g., be any one of the polypeptides disclosed herein.

Reference molecules (polypeptides or polynucleotides) may share a certain identity with the designed molecules (polypeptides or polynucleotides). The term “identity” as known in the art, refers to a relationship between the sequences of two or more peptides, polypeptides or polynucleotides, as determined by comparing the sequences. In the art, identity also means the degree of sequence relatedness between them as determined by the number of matches between strings of two or more amino acid residues or nucleosides. Identity measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

In some embodiments, the encoded polypeptide variant may have the same or a similar activity as the reference polypeptide. Alternatively, the variant may have an altered activity (e.g., increased or decreased) relative to a reference polypeptide. Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, Thomas L. Madden, Alejandro A. Schïffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.) Other tools are described herein, specifically in the definition of “Identity.”

Default parameters in the BLAST algorithm include, for example, an expect threshold of 10, Word size of 28, Match/Mismatch Scores 1, -2, Gap costs Linear. Any filter can be applied as well as a selection for species specific repeats, e.g., Homo sapiens.

### Cell-Penetrating Polypeptides

The polynucleotides disclosed herein, may also encode one or more cell-penetrating polypeptides. As used herein, “cell-penetrating polypeptide” or CPP refers to a polypeptide which may facilitate the cellular uptake of molecules. A cell-penetrating polypeptide of the present invention may contain one or more detectable labels. The polypeptides may be partially labeled or completely labeled throughout. The polynucleotides may encode the detectable label completely, partially or not at all. The cell-penetrating peptide may also include a signal sequence. As used herein, a “signal sequence” refers to a sequence of amino acid residues bound at the amino terminus of a nascent protein during protein translation. The signal sequence may be used to signal the secretion of the cell-penetrating polypeptide.

In one embodiment, the polynucleotides may also encode a fusion protein. The fusion protein may be created by operably linking a charged protein to a therapeutic protein. As used herein, “operably linked” refers to the therapeutic protein and the charged protein being connected in such a way to permit the expression of the complex when introduced into the cell. As used herein, “charged protein” refers to a protein that carries a positive, negative or overall neutral electrical charge. Preferably, the therapeutic protein may be covalently linked to the charged protein in the formation of the fusion protein. The ratio of surface charge to total or surface amino acids may be approximately 0.1, 0.2, 0.3, 0.4, 0.5, 0.6, 0.7, 0.8 or 0.9.

### Polypeptide Libraries

In one embodiment, the polynucleotides may be used to produce polypeptide libraries. These libraries may arise from the production of a population of polynucleotides, each containing various structural or chemical modification designs. In this embodiment, a population of polynucleotides may comprise a plurality of encoded polypeptides, including but not limited to, an antibody or antibody fragment, protein binding partner, scaffold protein, and other polypeptides taught herein or known in the art. In one embodiment, the polynucleotides may be suitable for direct introduction into a target cell or culture which in turn may synthesize the encoded polypeptides.

In certain embodiments, multiple variants of a protein, each with different amino acid modification(s), may be produced and tested to determine the best variant in terms of pharmacokinetics, stability, biocompatibility, and/or biological activity, or a biophysical property such as expression level. Such a library may contain 10, 10², 10³, 10⁴, 10⁵, 10⁶, 10⁷, 10⁸, 10⁹, or over 10⁹ possible variants (including, but not limited to, substitutions, deletions of one or more residues, and insertion of one or more residues).

### Cytotoxic Nucleosides

In one embodiment, the polynucleotides of the present invention may incorporate one or more cytotoxic nucleosides. For example, cytotoxic nucleosides may be incorporated into polynucleotides such as bifunctional modified RNAs or mRNAs. Cytotoxic nucleoside anti-cancer agents include, but are not limited to, adenosine arabinoside, cytarabine, cytosine arabinoside, 5-fluorouracil, fludarabine, floxuridine, FTORAFUR® (a combination of tegafur and uracil), tegafur ((RS)-5-fluoro-1-(tetrahydrofuran-2-yl)pyrimidine-2,4(1H,3H)-dione), and 6-mercaptopurine.

A number of cytotoxic nucleoside analogues are in clinical use, or have been the subject of clinical trials, as anticancer agents. Examples of these and other cytotoxic nucleosides may be found in copending International application number PCT/US2014/069155 (Attorney Docket No. M073), the contents of which are incorporated herein by reference in their entirety and may be used in the present invention.

### Polynucleotides Having Untranslated Regions (UTRs)

The polynucleotides of the present invention may comprise one or more regions or parts which act or function as an untranslated region. Where polynucleotides are designed to encode at least one polypeptide of interest, the polynucleotides may comprise one or more of these untranslated regions.

By definition, wild type untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present invention to, among other things, enhance the stability of the molecule. The specific features can also be incorporated to ensure controlled down-regulation of the transcript in case they are misdirected to undesired organs sites.

Tables 2 and 3 of copending International publication number WO2013151666, filed Mar. 9, 2013 (Attorney Docket No. M300) the contents of which are incorporated by reference in their entirety, provide a listing of exemplary UTRs which may be utilized in the polynucleotides of the present invention.

### 5′ UTR and Translation Initiation

Natural 5′UTRs bear features which play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

By engineering the features typically found in abundantly expressed genes of specific target organs, one can enhance the stability and protein production of the polynucleotides of the invention.

Untranslated regions useful in the design and manufacture of polynucleotides include, but are not limited, to those disclosed in co-pending, co-owned International Publication No. WO2014164253, the contents of which are incorporated herein by reference in their entirety.

Other non-UTR sequences may also be used as regions or subregions within the polynucleotides. For example, introns or portions of introns sequences may be incorporated into regions of the polynucleotides of the invention. Incorporation of intronic sequences may increase protein production as well as polynucleotide levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′UTR may comprise a first polynucleotide fragment and a second polynucleotide fragment from the same and/or different genes such as the 5′UTRs described in US Patent Application Publication No. 20100293625, herein incorporated by reference in its entirety.

Co-pending, co-owned International Publication Number WO2014164253 (Attorney Docket No. M042), the contents of which is herein incorporated by reference in its entirety, provides a listing of exemplary UTRs which may be utilized in the polynucleotide of the present invention as flanking regions. Variants of 5′ or 3′ UTRs may be utilized wherein one or more nucleotides are added or removed to the termini, including A, T, C or G.

It should be understood that any UTR from any gene may be incorporated into the regions of the polynucleotide. Furthermore, multiple wild-type UTRs of any known gene may be utilized. It is also within the scope of the present invention to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

The untranslated region may also include translation enhancer elements (TEE). As a non-limiting example, the TEE may include those described in US Application No. 20090226470, herein incorporated by reference in its entirety, and those known in the art.

### 3′ UTR and the AU Rich Elements

Natural or wild type 3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides of the invention. When engineering specific polynucleotides, one or more copies of an ARE can be introduced to make polynucleotides of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using polynucleotides of the invention and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

microRNA Binding Sites

microRNAs (or miRNA) are 19-25 nucleotide long noncoding RNAs that bind to the 3′UTR of nucleic acid molecules and down-regulate gene expression either by reducing nucleic acid molecule stability or by inhibiting translation. The polynucleotides of the invention may comprise one or more microRNA target sequences, microRNA sequences, or microRNA seeds. Such sequences may correspond to any known microRNA such as those taught in US Publication US2005/0261218 and US Publication US2005/0059005, the contents of which are incorporated herein by reference in their entirety.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked byan adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. The bases of the microRNA seed have complete complementarity with the target sequence. By engineering microRNA target sequences into the polynucleotides (e.g., in a 3′UTR like region or other region) of the invention one can target the molecule for degradation or reduced translation, provided the microRNA in question is available. This process will reduce the hazard of off target effects upon nucleic acid molecule delivery. Identification of microRNA, microRNA target regions, and their expression patterns and role in biology have been reported (Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu. 2011.356); Bartel Cell 2009 136:215-233; Landgraf et al, Cell, 2007 129:1401-1414; each of which is herein incorporated by reference in its entirety).

As used herein, the term “microRNA site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). MicroRNA can also regulate complex biological processes such as angiogenesis (miR-132) (Anand and Cheresh Curr Opin Hematol 2011 18:171-176; herein incorporated by reference in its entirety).

Expression profiles, microRNA and cell lines useful in the present invention include those taught in, for example, International Publication No. WO2014081507 (Attorney Docket No. M039) and International Publication No. WO2014113089 (Attorney Docket Number M37), the contents of each of which are incorporated by reference in their entirety.

In the polynucleotides of the present invention, binding sites for microRNAs that are involved in such processes may be removed or introduced, in order to tailor the expression of the polynucleotides expression to biologically relevant cell types or to the context of relevant biological processes. A listing of microRNA, miR sequences and miR binding sites is listed in Table 9 of U.S. Provisional Application No. 61/753,661 filed Jan. 17, 2013, in Table 9 of U.S. Provisional Application No. 61/754,159 filed Jan. 18, 2013, and in Table 7 of U.S. Provisional Application No. 61/758,921 filed Jan. 31, 2013, each of which are herein incorporated by reference in their entireties.

Examples of use of microRNA to drive tissue or disease-specific gene expression are listed (Getner and Naldini, Tissue Antigens. 2012, 80:393-403; herein incorporated by reference in its entirety). In addition, microRNA seed sites can be incorporated into mRNA to decrease expression in certain cells which results in a biological improvement.

Lastly, through an understanding of the expression patterns of microRNA in different cell types, polynucleotides can be engineered for more targeted expression in specific cell types or only under specific biological conditions. Through introduction of tissue-specific microRNA binding sites, polynucleotides could be designed that would be optimal for protein expression in a tissue or in the context of a biological condition.

Transfection experiments can be conducted in relevant cell lines, using engineered polynucleotides and protein production can be assayed at various time points post-transfection. For example, cells can be transfected with different microRNA binding site-engineering polynucleotides and by using an ELISA kit to the relevant protein and assaying protein produced at 6 hour, 12 hour, 24 hour, 48 hour, 72 hour and 7 days post-transfection. In vivo experiments can also be conducted using microRNA-binding site-engineered molecules to examine changes in tissue-specific expression of formulated polynucleotides.

### Regions Having a 5′ Cap

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

In some embodiments, polynucleotides may be designed to incorporate a cap moiety. Modifications to the polynucleotides of the present invention may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′O-Me-m7G(5)ppp(5′)G). The 3′-O atom of the other, unmodified, guanine becomes linked to the 5′-terminal nucleotide of the capped polynucleotide. The N7- and 3′-O-methylated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In one embodiment, the cap is a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dicucleotide form of a cap analog known in the art and/or described herein. Non-limiting examples of a N7-(4-chlorophenoxyethyl) substituted dicucleotide form of a cap analog include a N7-(4-chlorophenoxyethyl)-G(5′)ppp(5′)G and a N7-(4-chlorophenoxyethyl)-m^{3′-O}G(5′)ppp(5′)G cap analog (See e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

Polynucleotides of the invention may also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those which, among other things, have enhanced binding of cap binding proteins, increased half life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N, pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture may be more efficient as nearly 100% of the chimeric polynucleotides may be capped. This is in contrast to ˜80% when a cap analog is linked to a chimeric polynucleotide in the course of an in vitro transcription reaction.

According to the present invention, 5′ terminal caps may include endogenous caps or cap analogs. According to the present invention, a 5′ terminal cap may comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Viral Sequences

Additional viral sequences such as, but not limited to, the translation enhancer sequence of the barley yellow dwarf virus (BYDV-PAV), the Jaagsiekte sheep retrovirus (JSRV) and/or the Enzootic nasal tumor virus (See e.g., International Pub. No. WO2012129648; herein incorporated by reference in its entirety) can be engineered and inserted in the polynucleotides of the invention and can stimulate the translation of the construct in vitro and in vivo. Transfection experiments can be conducted in relevant cell lines at and protein production can be assayed by ELISA at 12 hr, 24 hr, 48 hr, 72 hr and day 7 post-transfection.

### IRES Sequences

Further, provided are polynucleotides which may contain an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA. Polynucleotides containing more than one functional ribosome binding site may encode several peptides or polypeptides that are translated independently by the ribosomes (“multicistronic nucleic acid molecules”). When polynucleotides are provided with an IRES, further optionally provided is a second translatable region. Examples of IRES sequences that can be used according to the invention include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Poly-A Tails

During RNA processing, a long chain of adenine nucleotides (poly-A tail) may be added to a polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long (SEQ ID NO: 393), including approximately 80, 90, 100, 110, 120, 130, 140, 150, 160, 170, 180, 190, 200, 210, 220, 230, 240 or 250 residues long.

According to the present invention, terminal groups on the poly A tail may be incorporated for stabilization. Polynucleotides of the present invention may include des-3′ hydroxyl tails. They may also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention may be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention.

Generally, the length of a poly-A tail, when present, is greater than 30 nucleotides in length (SEQ ID NO: 394). In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In one embodiment, the poly-A tail is designed relative to the length of the overall polynucleotide or the length of a particular region of the polynucleotide. This design may be based on the length of a coding region, the length of a particular feature or region or based on the length of the ultimate product expressed from the polynucleotides.

In this context the poly-A tail may be 10, 20, 30, 40, 50, 60, 70, 80, 90, or 100% greater in length than the polynucleotide or feature thereof. The poly-A tail may also be designed as a fraction of the polynucleotides to which it belongs. In this context, the poly-A tail may be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein may enhance expression.

Additionally, multiple distinct polynucleotides may be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In one embodiment, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides (SEQ ID NO: 395) alone.

### Start Codon Region

In some embodiments, the polynucleotides of the present invention may have regions that are analogous to or function like a start codon region.

In one embodiment, the translation of a polynucleotide may initiate on a codon which is not the start codon AUG. Translation of the polynucleotide may initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety). As a non-limiting example, the translation of a polynucleotide begins on the alternative start codon ACG. As another non-limiting example, polynucleotide translation begins on the alternative start codon CTG or CUG. As yet another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See e.g., Matsuda and Mauro PLoS ONE, 2010 5:11; the contents of which are herein incorporated by reference in its entirety). Masking any of the nucleotides flanking a codon that initiates translation may be used to alter the position of translation initiation, translation efficiency, length and/or structure of a polynucleotide.

In one embodiment, a masking agent may be used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See e.g., Matsuda and Mauro describing masking agents LNA polynucleotides and EJCs (PLoS ONE, 2010 5:11); the contents of which are herein incorporated by reference in its entirety).

In another embodiment, a masking agent may be used to mask a start codon of a polynucleotide in order to increase the likelihood that translation will initiate on an alternative start codon.

In one embodiment, a masking agent may be used to mask a first start codon or alternative start codon in order to increase the chance that translation will initiate on a start codon or alternative start codon downstream to the masked start codon or alternative start codon.

In one embodiment, a start codon or alternative start codon may be located within a perfect complement for a miR binding site. The perfect complement of a miR binding site may help control the translation, length and/or structure of the polynucleotide similar to a masking agent. As a non-limiting example, the start codon or alternative start codon may be located in the middle of a perfect complement for a miR-122 binding site. The start codon or alternative start codon may be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide may be removed from the polynucleotide sequence in order to have the translation of the polynucleotide begin on a codon which is not the start codon. Translation of the polynucleotide may begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed may further comprise at least one masking agent for the downstream start codon and/or alternative start codons in order to control or attempt to control the initiation of translation, the length of the polynucleotide and/or the structure of the polynucleotide.

### Stop Codon Region

In one embodiment, the polynucleotides of the present invention may include at least two stop codons before the 3′ untranslated region (UTR). The stop codon may be selected from TGA, TAA and TAG. In one embodiment, the polynucleotides of the present invention include the stop codon TGA and one additional stop codon. In a further embodiment the addition stop codon may be TAA. In another embodiment, the polynucleotides of the present invention include three stop codons.

### Signal Sequences

The polynucleotides may also encode additional features which facilitate trafficking of the polypeptides to therapeutically relevant sites. One such feature which aids in protein trafficking is the signal sequence. As used herein, a “signal sequence” or “signal peptide” is a polynucleotide or polypeptide, respectively, which is from about 9 to 200 nucleotides (3-60 amino acids) in length which is incorporated at the 5′ (or N-terminus) of the coding region or polypeptide encoded, respectively. Addition of these sequences result in trafficking of the encoded polypeptide to the endoplasmic reticulum through one or more secretory pathways. Some signal peptides are cleaved from the protein by signal peptidase after the proteins are transported.

Additional signal sequences which may be utilized in the present invention include those taught in, for example, databases such as those found at www.signalpeptide.de/ or proline.bic.nus.edu.sg/spdb/. Those described in U.S. Pat. Nos. 8,124,379; 7,413,875 and 7,385,034 are also within the scope of the invention and the contents of each are incorporated herein by reference in their entirety.

### Protein Cleavage Signals and Sites

In one embodiment, the polynucleotides may encode or the polypeptides of the present invention may include at least one protein cleavage signal containing at least one protein cleavage site. The protein cleavage site may be located at the N-terminus, the C-terminus, at any space between the N- and the C-termini such as, but not limited to, half-way between the N- and C-termini, between the N-terminus and the half way point, between the half way point and the C-terminus, and combinations thereof.

In one embodiment, the polynucleotides of the present invention may be engineered such that the polynucleotide contains at least one encoded protein cleavage signal. The encoded protein cleavage signal may be located in any region including but not limited to before the start codon, after the start codon, before the coding region, within a coding region such as, but not limited to, half way in the coding region, between the start codon and the half way point, between the half way point and the stop codon, after the coding region, before the stop codon, between two stop codons, after the stop codon and combinations thereof.

In one embodiment, the polynucleotides of the present invention may include at least one encoded protein cleavage signal containing at least one protein cleavage site. The encoded protein cleavage signal may include, but is not limited to, a proprotein convertase (or prohormone convertase), thrombin and/or Factor Xa protein cleavage signal.

As a non-limiting example, U.S. Pat. No. 7,374,930 and U.S. Pub. No. 20090227660, herein incorporated by reference in their entireties, use a furin cleavage site and such sites are useful in the polynucleotides of the present invention.

### Insertions and Substitutions

In one embodiment, the 5′UTR of the polynucleotide may be replaced by the insertion of at least one region and/or string of nucleosides of the same base. The region and/or string of nucleotides may include, but is not limited to, at least 3, at least 4, at least 5, at least 6, at least 7 or at least 8 nucleotides and the nucleotides may be natural and/or unnatural. As a non-limiting example, the group of nucleotides may include 5-8 adenine, cytosine, thymine, a string of any of the other nucleotides disclosed herein and/or combinations thereof.

In one embodiment, the 5′UTR of the polynucleotide may be replaced by the insertion of at least two regions and/or strings of nucleotides of two different bases such as, but not limited to, adenine, cytosine, thymine, any of the other nucleotides disclosed herein and/or combinations thereof. For example, the 5′UTR may be replaced by inserting 5-8 adenine bases followed by the insertion of 5-8 cytosine bases. In another example, the 5′UTR may be replaced by inserting 5-8 cytosine bases followed by the insertion of 5-8 adenine bases.

In one embodiment, the polynucleotide may include at least one substitution and/or insertion downstream of the transcription start site which may be recognized by an RNA polymerase. As a non-limiting example, at least one substitution and/or insertion may occur downstream the transcription start site by substituting at least one nucleic acid in the region just downstream of the transcription start site (such as, but not limited to, +1 to +6). Changes to region of nucleotides just downstream of the transcription start site may affect initiation rates, increase apparent nucleotide triphosphate (NTP) reaction constant values, and increase the dissociation of short transcripts from the transcription complex curing initial transcription (Brieba et al, Biochemistry (2002) 41: 5144-5149; herein incorporated by reference in its entirety). The modification, substitution and/or insertion of at least one nucleoside may cause a silent mutation of the sequence or may cause a mutation in the amino acid sequence.

In one embodiment, the polynucleotide may include the substitution of at least 1, at least 2, at least 3, at least 4, at least 5, at least 6, at least 7, at least 8, at least 9, at least 10, at least 11, at least 12 or at least 13 guanine bases downstream of the transcription start site.

In one embodiment, the polynucleotide may include the substitution of at least 1, at least 2, at least 3, at least 4, at least 5 or at least 6 guanine bases in the region just downstream of the transcription start site. As a non-limiting example, if the nucleotides in the region are GGGAGA the guanine bases may be substituted by at least 1, at least 2, at least 3 or at least 4 adenine nucleotides. In another non-limiting example, if the nucleotides in the region are GGGAGA the guanine bases may be substituted by at least 1, at least 2, at least 3 or at least 4 cytosine bases. In another non-limiting example, if the nucleotides in the region are GGGAGA the guanine bases may be substituted by at least 1, at least 2, at least 3 or at least 4 thymine, and/or any of the nucleotides described herein.

In one embodiment, the polynucleotide may include at least one substitution and/or insertion upstream of the start codon. For the purpose of clarity, one of skill in the art would appreciate that the start codon is the first codon of the protein coding region whereas the transcription start site is the site where transcription begins. The polynucleotide may include, but is not limited to, at least 1, at least 2, at least 3, at least 4, at least 5, at least 6, at least 7 or at least 8 substitutions and/or insertions of nucleotide bases. The nucleotide bases may be inserted or substituted at 1, at least 1, at least 2, at least 3, at least 4 or at least 5 locations upstream of the start codon. The nucleotides inserted and/or substituted may be the same base (e.g., all A or all C or all T or all G), two different bases (e.g., A and C, A and T, or C and T), three different bases (e.g., A, C and T or A, C and T) or at least four different bases. As a non-limiting example, the guanine base upstream of the coding region in the polynucleotide may be substituted with adenine, cytosine, thymine, or any of the nucleotides described herein. In another non-limiting example the substitution of guanine bases in the polynucleotide may be designed so as to leave one guanine base in the region downstream of the transcription start site and before the start codon (see Esvelt et al. Nature (2011) 472(7344):499-503; the contents of which is herein incorporated by reference in its entirety). As a non-limiting example, at least 5 nucleotides may be inserted at 1 location downstream of the transcription start site but upstream of the start codon and the at least 5 nucleotides may be the same base type.

### Incorporating Post Transcriptional Control Modulators

In one embodiment, the polynucleotides of the present invention may include at least one post transcriptional control modulator. These post transcriptional control modulators may be, but are not limited to, small molecules, compounds and regulatory sequences. As a non-limiting example, post transcriptional control may be achieved using small molecules identified by PTC Therapeutics Inc. (South Plainfield, N.J.) using their GEMS' (Gene Expression Modulation by Small-Molecules) screening technology.

The post transcriptional control modulator may be a gene expression modulator which is screened by the method detailed in or a gene expression modulator described in International Publication No. WO2006022712, herein incorporated by reference in its entirety. Methods identifying RNA regulatory sequences involved in translational control are described in International Publication No. WO2004067728, herein incorporated by reference in its entirety; methods identifying compounds that modulate untranslated region dependent expression of a gene are described in International Publication No. WO2004065561, herein incorporated by reference in its entirety.

In one embodiment, the polynucleotides of the present invention may include at least one post transcriptional control modulator is located in the 5′ and/or the 3′ untranslated region of the polynucleotides of the present invention.

In another embodiment, the polynucleotides of the present invention may include at least one post transcription control modulator to modulate premature translation termination. The post transcription control modulators may be compounds described in or a compound found by methods outlined in International Publication Nso. WO2004010106, WO2006044456, WO2006044682, WO2006044503 and WO2006044505, each of which is herein incorporated by reference in its entirety. As a non-limiting example, the compound may bind to a region of the 28S ribosomal RNA in order to modulate premature translation termination (See e.g., WO2004010106, herein incorporated by reference in its entirety).

In one embodiment, polynucleotides of the present invention may include at least one post transcription control modulator to alter protein expression. As a non-limiting example, the expression of VEGF may be regulated using the compounds described in or a compound found by the methods described in International Publication Nos. WO2005118857, WO2006065480, WO2006065479 and WO2006058088, each of which is herein incorporated by reference in its entirety.

The polynucleotides of the present invention may include at least one post transcription control modulator to control translation. In one embodiment, the post transcription control modulator may be a RNA regulatory sequence. As a non-limiting example, the RNA regulatory sequence may be identified by the methods described in International Publication No. WO2006071903, herein incorporated by reference in its entirety.

### II. DESIGN, SYNTHESIS AND QUANTITATION OF POLYNUCLEOTIDES

### Codon Optimization

The polynucleotides of the invention, their regions or parts or subregions may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide. Codon optimization tools, algorithms and services are known in the art, non-limiting examples include services from GeneArt (Life Technologies), DNA2.0 (Menlo Park Calif.) and/or proprietary methods. In one embodiment, the ORF sequence is optimized using optimization algorithms. Codon options for each amino acid are given in Table 75.

TABLE 75
Codon Options

| | Single | |
|---|---|---|
| | Letter | |
| Amino Acid | Code | Codon Options |
| Isoleucine | I | ATT, ATC, ATA |
| Leucine | L | CTT, CTC, CTA, CTG, TTA, TTG |
| Valine | V | GTT, GTC, GTA, GTG |
| Phenylalanine | F | TTT, TTC |
| Methionine | M | ATG |
| Cysteine | C | TGT, TGC |
| Alanine | A | GCT, GCC, GCA, GCG |
| Glycine | G | GGT, GGC, GGA, GGG |
| Proline | P | CCT, CCC, CCA, CCG |
| Threonine | T | ACT, ACC, ACA, ACG |
| Serine | S | TCT, TCC, TCA, TCG, AGT, AGC |
| Tyrosine | Y | TAT, TAC |
| Tryptophan | W | TGG |
| Glutamine | Q | CAA, CAG |
| Asparagine | N | AAT, AAC |
| Histidine | H | CAT, CAC |
| Glutamic acid | E | GAA, GAG |
| Aspartic acid | D | GAT, GAC |
| Lysine | K | AAA, AAG |
| Arginine | R | CGT, CGC, CGA, CGG, AGA, AGG |
| Selenocysteine | Sec | UGA in mRNA in presence of Selenocystein |
| | | insertion element (SECIS) |
| Stop codons | Stop | TAA, TAG, TGA |

Features, which may be considered beneficial in some embodiments of the present invention, may be encoded by regions of the polynucleotide and such regions may be upstream (5′) or downstream (3′) to a region which encodes a polypeptide. These regions may be incorporated into the polynucleotide before and/or after codon optimization of the protein encoding region or open reading frame (ORF). It is not required that a polynucleotide contain both a 5′ and 3′ flanking region. Examples of such features include, but are not limited to, untranslated regions (UTRs), Kozak sequences, an oligo(dT) sequence, and detectable tags and may include multiple cloning sites which may have XbaI recognition.

### Synthesis

Enzymatic (IVT), solid-phase, liquid-phase, combined synthetic methods, small region synthesis, and ligation methods are taught in for example copending International application number PCT/US2014/069155 (Attorney Docket Number M073), the contents of which are incorporated herein by reference in their entirety, and may be utilized to manufacture the polynucleotides of the present invention.

### Modified and Conjugated Polynucleotides

Non-natural modified nucleotides may be introduced to polynucleotides or nucleic acids during synthesis or post-synthesis of the chains to achieve desired functions or properties. The modifications may be on internucleotide lineage, the purine or pyrimidine bases, or sugar. The modification may be introduced at the terminal of a chain or anywhere else in the chain; with chemical synthesis or with a polymerase enzyme. For example, hexitol nucleic acids (HNAs) are nuclease resistant and provide strong hybridization to RNA. Short messenger RNAs (mRNAs) with hexitol residues in two codons have been constructed (Lavrik et al., Biochemistry, 40, 11777-11784 (2001), the contents of which are incorporated herein by reference in their entirety). The antisense effects of a chimeric HNA gapmer oligonucleotide comprising a phosphorothioate central sequence flanked by 5′ and 3′ HNA sequences have also been studied (See e.g., Kang et al., Nucleic Acids Research, vol. 32(4), 4411-4419 (2004), the contents of which are incorporated herein by reference in their entirety). The preparation and uses of modified nucleotides comprising 6-member rings in RNA interference, antisense therapy or other applications are disclosed in US Pat. Application No. 2008/0261905, US Pat. Application No. 2010/0009865, and PCT Application No. WO97/30064 to Herdewijn et al.; the contents of each of which are herein incorporated by reference in their entireties). Modified nucleic acids and their synthesis are disclosed in copending PCT applications No. PCT/US2012/058519 (Attorney Docket Number M09), the contents of which are incorporated herein by reference for their entirety. The synthesis and strategy of modified polynucleotides is reviewed by Verma and Eckstein in Annual Review of Biochemistry, vol. 76, 99-134 (1998), the contents of which are incorporated herein by reference in their entirety.

Either enzymatic or chemical ligation methods can be used to conjugate polynucleotides or their regions with different functional blocks, such as fluorescent labels, liquids, nanoparticles, delivery agents, etc. The conjugates of polynucleotides and modified polynucleotides are reviewed by Goodchild in Bioconjugate Chemistry, vol. 1(3), 165-187 (1990), the contents of which are incorporated herein by reference in their entirety. U.S. Pat. No. 6,835,827 and U.S. Pat. No. 6,525,183 to Vinayak et al. (the contents of each of which are herein incorporated by reference in their entireties) teach synthesis of labeled oligonucleotides using a labeled solid support.

### Quantification

In one embodiment, the polynucleotides of the present invention may be quantified in exosomes or when derived from one or more bodily fluid. As used herein “bodily fluids” include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Quantification may be by size exclusion chromatography, density gradient centrifugation, differential centrifugation, nanomembrane ultrafiltration, immunoabsorbent capture, affinity purification, microfluidic separation, or combinations thereof. In the analysis, the level or concentration of a polynucleotide may be an expression level, presence, absence, truncation or alteration of the administered construct.

It is often advantageous to correlate the level with one or more clinical phenotypes or with an assay for a human disease biomarker. The assay may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of polynucleotides remaining or delivered. This is possible because the polynucleotides of the present invention differ from the endogenous forms due to the structural or chemical modifications.

In one embodiment, the polynucleotide may be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified polynucleotide may be analyzed in order to determine if the polynucleotide may be of proper size, check that no degradation of the polynucleotide has occurred. Degradation of the polynucleotide may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Purification

Purification of the polynucleotides described herein may include, but is not limited to, polynucleotide clean-up, quality assurance and quality control. Clean-up may be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In another embodiment, the polynucleotides may be sequenced by methods including, but not limited to reverse-transcriptase-PCR.

### III. MODIFICATIONS

As used herein in a polynucleotide (such as a chimeric polynucleotide, IVT polynucleotide or a circular polynucleotide), the terms “chemical modification” or, as appropriate, “chemically modified” refer to modification with respect to adenosine (A), guanosine (G), uridine (U), thymidine (T) or cytidine (C) ribo- or deoxyribnucleosides in one or more of their position, pattern, percent or population. Generally, herein, these terms are not intended to refer to the ribonucleotide modifications in naturally occurring 5′-terminal mRNA cap moieties.

In a polypeptide, the term “modification” refers to a modification as compared to the canonical set of 20 amino acids.

The modifications may be various distinct modifications. In some embodiments, the regions may contain one, two, or more (optionally different) nucleoside or nucleotide modifications. In some embodiments, a modified polynucleotide, introduced to a cell may exhibit reduced degradation in the cell, as compared to an unmodified polynucleotide.

Modifications of the polynucleotides of the antibody compositions which are useful in the present invention include, but are not limited to those in Tables 5, 6 or the linkers of Table 7 of copending application U.S. 61/912,635 filed Dec. 6, 2013 (Attorney Docket Number M073.60) or any of the modifications, both naturally occurring and non-naturally occurring) described in copending International Application Number PCT/2012/058519 filed Oct. 3, 2012 (Attorney Docket Number M9) and U.S. Provisional Application No. 61/837,297 filed Jun. 20, 2013 (Attorney Docket Number M36), U.S. Provisional Application No. 61/886,006 filed Oct. 2, 2013 (Attorney Docket Number M71), U.S. Provisional Application No. 61/896,478 filed Oct. 28, 2013 (Attorney Docket Number M72), and U.S. Provisional Application No. 61/916,052 filed Dec. 13, 2013 (Attorney Docket Number M79), the contents of each of which are incorporated herein by reference in their entireties.

The polynucleotides can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

### Modified Polynucleotide Molecules

The present invention also includes building blocks, e.g., modified ribonucleosides, and modified ribonucleotides, of polynucleotide molecules. For example, these building blocks can be useful for preparing the polynucleotides of the invention. Such building blocks are taught in International Application Number PCT/2012/058519 filed Oct. 3, 2012 (Attorney Docket Number M9) and U.S. Provisional Application No. 61/837,297 filed Jun. 20, 2013 (Attorney Docket Number M36), U.S. Provisional Application No. 61/886,006 filed Oct. 2, 2013 (Attorney Docket Number M71), U.S. Provisional Application No. 61/896,478 filed Oct. 28, 2013 (Attorney Docket Number M72), and U.S. Provisional Application No. 61/916,052 filed Dec. 13, 2013 (Attorney Docket Number M79), the contents of each of which are incorporated herein by reference in its entirety.

### Combinations of Modified Sugars, Nucleobases, and Internucleoside Linkages

The polynucleotides of the invention can include a combination of modifications to the sugar, the nucleobase, and/or the internucleoside linkage. These combinations can include any one or more modifications described herein.

Examples of modified nucleotides and modified nucleotide combinations are provided, for example in Table 8 of copending application U.S. 61/912,635 filed Dec. 6, 2013 (Attorney Docket Number M073.60) or any of those disclosed in copending International Application Number PCT/2012/058519 filed Oct. 3, 2012 (Attorney Docket Number M9), U.S. Provisional Application No. 61/837,297 filed Jun. 20, 2013 (Attorney Docket Number M36), U.S. Provisional Application No. 61/886,006 filed Oct. 2, 2013 (Attorney Docket Number M71), U.S. Provisional Application No. 61/896,478 filed Oct. 28, 2013 (Attorney Docket Number M72), and U.S. Provisional Application No. 61/916,052 filed Dec. 13, 2013 (Attorney Docket Number M79), the contents of each of which are incorporated herein by reference in its entirety.

### IV. PHARMACEUTICAL COMPOSITIONS

### Formulation, Administration, Delivery and Dosing

The present invention provides polynucleotides, antibody compositions and complexes optionally in combination with one or more pharmaceutically acceptable excipients. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference in its entirety).

In some embodiments, compositions are administered to humans, human patients or subjects. For the purposes of the present disclosure, the phrase “active ingredient” generally refers to the antibody composition or the polynucleotides contained therein to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

### Formulations

The antibody compositions of the invention can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antibody) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with antibody compositions (e.g., for transplantation into a subject), hyaluronidase, nanoparticle mimics and combinations thereof. Each of the foregoing methods is described in copending International application No PCT/US2014/069155 filed Dec. 8, 2014 (Attorney Docket Number M073), International Application PCT/US2012/69610, filed Dec. 14, 2012 (Attorney Docket number M11) and International Application No. PCT/US2014/027077 (Attorney Docket Number M030), the contents of each of which are incorporated herein by reference in their entireties.

Accordingly, the formulations of the invention can include one or more excipients, each in an amount that may increases the stability of the antibody composition, increases cell transfection by the antibody composition, increases the expression of polynucleotides encoded protein, and/or alters the release profile of polynucleotide encoded proteins. Further, the polynucleotides of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the formulations described herein may contain at least one polynucleotide. As a non-limiting example, the formulations may contain 1, 2, 3, 4 or 5 polynucleotides.

In one embodiment, the formulations described herein may comprise more than one type of polynucleotide. In one embodiment, the formulation may comprise a chimeric polynucleotide in linear and circular form. In another embodiment, the formulation may comprise a circular polynucleotide and an IVT polynucleotide. In yet another embodiment, the formulation may comprise an IVT polynucleotide, a chimeric polynucleotide and a circular polynucleotide.

In one embodiment, the formulation contains at least three polynucleotides encoding proteins. In one embodiment, the formulation contains at least five polynucleotide encoding proteins.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

### Lipidoids

The synthesis of lipidoids has been extensively described and formulations containing these compounds are particularly suited for delivery of polynucleotides (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

Complexes, micelles, liposomes or particles can be prepared containing these lipidoids and therefore, can result in an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intramuscular, or subcutaneous routes.

In vivo delivery of nucleic acids may be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009 17:872-879; herein incorporated by reference in its entirety). As an example, small changes in the anchor chain length of poly(ethylene glycol) (PEG) lipids may result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010); herein incorporated by reference in its entirety), C12-200 (including derivatives and variants), and MD1, can be tested for in vivo activity.

The lipidoid referred to herein as “98N12-5” is disclosed by Akinc et al., Mol Ther. 2009 17:872-879 and is incorporated by reference in its entirety.

The lipidoid referred to herein as “C12-200” is disclosed by Love et al., Proc Natl Acad Sci USA. 2010 107:1864-1869 and Liu and Huang, Molecular Therapy. 2010 669-670; both of which are herein incorporated by reference in their entirety. The lipidoid formulations can include particles comprising either 3 or 4 or more components in addition to polynucleotides. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In one embodiment, a polynucleotide formulated with a lipidoid for systemic intravenous administration can target the liver. For example, a final optimized intravenous formulation using polynucleotides, and comprising a lipid molar composition of 42% 98N12-5, 48% cholesterol, and 10% PEG-lipid with a final weight ratio of about 7.5 to 1 total lipid to polynucleotides, and a C14 alkyl chain length on the PEG lipid, with a mean particle size of roughly 50-60 nm, can result in the distribution of the formulation to be greater than 90% to the liver. (see, Akinc et al., Mol Ther. 2009 17:872-879; herein incorporated by reference in its entirety). In another example, an intravenous formulation using a C12-200 (see U.S. provisional application 61/175,770 and published international application WO2010129709, each of which is herein incorporated by reference in their entirety) lipidoid may have a molar ratio of 50/10/38.5/1.5 of C12-200/disteroylphosphatidyl choline/cholesterol/PEG-DMG, with a weight ratio of 7 to 1 total lipid to polynucleotides, and a mean particle size of 80 nm may be effective to deliver polynucleotides to hepatocytes (see, Love et al., Proc Natl Acad Sci USA. 2010 107:1864-1869 herein incorporated by reference in its entirety). In another embodiment, an MD1 lipidoid-containing formulation may be used to effectively deliver polynucleotides to hepatocytes in vivo.

The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae (see, Akinc et al., Mol Ther. 2009 17:872-879 herein incorporated by reference in its entirety), use of a lipidoid-formulated antibody compositions to deliver the formulation to other cells types including, but not limited to, endothelial cells, myeloid cells, and muscle cells may not be similarly size-limited.

Use of lipidoid formulations to deliver siRNA in vivo to other non-hepatocyte cells such as myeloid cells and endothelium has been reported (see Akinc et al., Nat Biotechnol. 2008 26:561-569; Leuschner et al., Nat Biotechnol. 2011 29:1005-1010; Cho et al. Adv. Funct. Mater. 2009 19:3112-3118; 8^{th} International Judah Folkman Conference, Cambridge, Mass. Oct. 8-9, 2010; each of which is herein incorporated by reference in its entirety). Effective delivery to myeloid cells, such as monocytes, lipidoid formulations may have a similar component molar ratio. Different ratios of lipidoids and other components including, but not limited to, disteroylphosphatidyl choline, cholesterol and PEG-DMG, may be used to optimize the formulation of the antibody compositions for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and %1.5 PEG-DMG (see Leuschner et al., Nat Biotechnol 2011 29:1005-1010; herein incorporated by reference in its entirety). The use of lipidoid formulations for the localized delivery of nucleic acids to cells (such as, but not limited to, adipose cells and muscle cells) via either subcutaneous or intramuscular delivery, may not require all of the formulation components desired for systemic delivery, and as such may comprise only the lipidoid and the antibody composition.

Combinations of different lipidoids may be used to improve the efficacy of polynucleotides directed protein production as the lipidoids may be able to increase cell transfection by the antibody composition; and/or increase the translation of encoded protein (see Whitehead et al., Mol. Ther. 2011, 19:1688-1694, herein incorporated by reference in its entirety).

### Liposomes, Lipoplexes, and Lipid Nanoparticles

The antibody compositions of the invention can be formulated using one or more liposomes, lipoplexes, or lipid nanoparticles. In one embodiment, pharmaceutical compositions of antibody compositions include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, the contents of each of which are herein incorporated by reference in its entirety.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, liposome formulations may comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In a preferred embodiment, formulations may comprise a percentage of cholesterol selected from the group consisting of 28.5%, 31.5%, 33.5%, 36.5%, 37.0%, 38.5%, 39.0% and 43.5%. In some embodiments, formulations may comprise from about 5.0% to about 10.0% DSPC and/or from about 7.0% to about 15.0% DSPC.

In one embodiment, pharmaceutical compositions may include liposomes which may be formed to deliver polynucleotides which may encode at least one antibody or any other polypeptide of interest. The polynucleotides or compositions may be encapsulated by the liposome and/or it may be contained in an aqueous core which may then be encapsulated by the liposome (see International Pub. Nos. WO2012031046, WO2012031043, WO2012030901 and WO2012006378 and US Patent Publication No. US20130189351, US20130195969 and US20130202684; the contents of each of which are herein incorporated by reference in their entirety).

In another embodiment, liposomes may be formulated for targeted delivery. As a non-limiting example, the liposome may be formulated for targeted delivery to the liver. The liposome used for targeted delivery may include, but is not limited to, the liposomes described in and methods of making liposomes described in US Patent Publication No. US20130195967, the contents of which are herein incorporated by reference in its entirety.

In another embodiment, the polynucleotide which may encode protein, such as an antibody, fragment or variant thereof, may be formulated in a cationic oil-in-water emulsion where the emulsion particle comprises an oil core and a cationic lipid which can interact with the polynucleotide anchoring the molecule to the emulsion particle (see International Pub. No. WO2012006380; the contents of which are herein incorporated by reference in their entirety).

In one embodiment, the antibody compositions may be formulated in a water-in-oil emulsion comprising a continuous hydrophobic phase in which the hydrophilic phase is dispersed. As a non-limiting example, the emulsion may be made by the methods described in International Publication No. WO201087791, the contents of which are herein incorporated by reference in their entirety.

In another embodiment, the lipid formulation may include at least cationic lipid, a lipid which may enhance transfection and a least one lipid which contains a hydrophilic head group linked to a lipid moiety (International Pub. No. WO2011076807 and U.S. Pub. No. 20110200582; the contents of each of which is herein incorporated by reference in their entirety). In another embodiment, the polynucleotides encoding an immunogen may be formulated in a lipid vesicle which may have crosslinks between functionalized lipid bilayers (see U.S. Pub. No. 20120177724, the contents of which is herein incorporated by reference in its entirety).

In one embodiment, the polynucleotides may be formulated in a liposome as described in International Patent Publication No. WO2013086526, herein incorporated by reference in its entirety. The antibody compositions may be encapsulated in a liposome using reverse pH gradients and/or optimized internal buffer compositions as described in International Patent Publication No. WO2013086526, herein incorporated by reference in its entirety.

In one embodiment, the antibody compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In one embodiment, the cationic lipid may be a low molecular weight cationic lipid such as those described in US Patent Application No. 20130090372, the contents of which are herein incorporated by reference in its entirety.

In one embodiment, the antibody compositions may be formulated in a lipid vesicle which may have crosslinks between functionalized lipid bilayers.

In one embodiment, the antibody compositions may be formulated in a liposome comprising a cationic lipid. The liposome may have a molar ratio of nitrogen atoms in the cationic lipid to the phosphates in the RNA (N:P ratio) of between 1:1 and 20:1 as described in International Publication No. WO2013006825, herein incorporated by reference in its entirety. In another embodiment, the liposome may have a N:P ratio of greater than 20:1 or less than 1:1.

In one embodiment, the antibody compositions may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyornithine and/or polyarginine and the cationic peptides described in International Pub. No. WO2012013326 or US Patent Pub. No. US20130142818; each of which is herein incorporated by reference in its entirety. In another embodiment, the antibody compositions may be formulated in a lipid-polycation complex which may further include a neutral lipid such as, but not limited to, cholesterol or dioleoyl phosphatidylethanolamine (DOPE).

In one embodiment, the antibody compositions may be formulated in an aminoalcohol lipidoid. Aminoalcohol lipidoids which may be used in the present invention may be prepared by the methods described in U.S. Pat. No. 8,450,298, herein incorporated by reference in its entirety.

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety). In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to mRNA in liposomes may be from about 5:1 to about 20:1, from about 10:1 to about 25:1, from about 15:1 to about 30:1 and/or at least 30:1.

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain from about 0.5% to about 3.0%, from about 1.0% to about 3.5%, from about 1.5% to about 4.0%, from about 2.0% to about 4.5%, from about 2.5% to about 5.0% and/or from about 3.0% to about 6.0% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the antibody compositions may be formulated in a lipid nanoparticle such as those described in International Publication No. WO2012170930, herein incorporated by reference in its entirety.

In one embodiment, the antibody compositions formulation comprising the polynucleotide is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In another aspect, the lipid may be a cationic lipid such as, but not limited to, DLin-DMA, DLin-D-DMA, DLin-MC3-DMA, DLin-KC2-DMA, DODMA and amino alcohol lipids. The amino alcohol cationic lipid may be the lipids described in and/or made by the methods described in US Patent Publication No. US20130150625, herein incorporated by reference in its entirety. As a non-limiting example, the cationic lipid may be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and US20130225836; the contents of each of which are herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365, WO2012044638 and WO2013116126 or US Patent Publication No. US20130178541 and US20130225836; the contents of each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula 151-192 of U.S. Pat. No. 7,404,969 and formula I-VI of US Patent Publication No. US20100036115, formula I of US Patent Publication No US20130123338; each of which is herein incorporated by reference in their entirety.

As a non-limiting example, the cationic lipid may be selected from (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16, 19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21 Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21 Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1 S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2 S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoylo ctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)—N,N-dimethylnonacosa-11,20,2-trien-10-amine or a pharmaceutically acceptable salt or stereoisomer thereof.

In one embodiment, the lipid may be a cleavable lipid such as those described in International Publication No. WO2012170889, herein incorporated by reference in its entirety.

In another embodiment, the lipid may be a cationic lipid such as, but not limited to, Formula (I) of U.S. Patent Application No. US20130064894, the contents of which are herein incorporated by reference in its entirety.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724, WO201021865, WO2013086373 and WO2013086354; the contents of each of which are herein incorporated by reference in their entirety.

In another embodiment, the cationic lipid may be a trialkyl cationic lipid. Non-limiting examples of trialkyl cationic lipids and methods of making and using the trialkyl cationic lipids are described in International Patent Publication No. WO2013126803, the contents of which are herein incorporated by reference in its entirety.

In one embodiment, the LNP formulations of the antibody compositions may contain PEG-c-DOMG at 3% lipid molar ratio. In another embodiment, the LNP formulations antibody compositions may contain PEG-c-DOMG at 1.5% lipid molar ratio.

In one embodiment, the pharmaceutical compositions of the antibody compositions may include at least one of the PEGylated lipids described in International Publication No. WO2012099755, herein incorporated by reference.

In one embodiment, the LNP formulation may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294; herein incorporated by reference in its entirety).

In one embodiment, the LNP formulation may be formulated by the methods described in International Publication Nos. WO2011127255 or WO2008103276, the contents of each of which is herein incorporated by reference in their entirety. As a non-limiting example, the antibody compositions described herein may be encapsulated in LNP formulations as described in WO2011127255 and/or WO2008103276; each of which is herein incorporated by reference in their entirety.

In one embodiment, the antibody compositions described herein may be formulated in a nanoparticle to be delivered by a parenteral route as described in U.S. Pub. No. US20120207845; the contents of which are herein incorporated by reference in its entirety.

In one embodiment, the antibody compositions may be formulated in a lipid nanoparticle made by the methods described in US Patent Publication No US20130156845 or International Publication No WO2013093648 or WO2012024526, each of which is herein incorporated by reference in its entirety.

The lipid nanoparticles described herein may be made in a sterile environment by the system and/or methods described in US Patent Publication No. US20130164400, herein incorporated by reference in its entirety.

In one embodiment, the LNP formulation may be formulated in a nanoparticle such as a nucleic acid-lipid particle described in U.S. Pat. No. 8,492,359, the contents of which are herein incorporated by reference in its entirety. As a non-limiting example, the lipid particle may comprise one or more active agents or therapeutic agents; one or more cationic lipids comprising from about 50 mol % to about 85 mol % of the total lipid present in the particle; one or more non-cationic lipids comprising from about 13 mol % to about 49.5 mol % of the total lipid present in the particle; and one or more conjugated lipids that inhibit aggregation of particles comprising from about 0.5 mol % to about 2 mol % of the total lipid present in the particle. The nucleic acid in the nanoparticle may be the polynucleotides described herein and/or are known in the art.

In one embodiment, the LNP formulation may be formulated by the methods described in International Publication Nos. WO2011127255 or WO2008103276, the contents of each of which are herein incorporated by reference in their entirety. As a non-limiting example, modified RNA described herein may be encapsulated in LNP formulations as described in WO2011127255 and/or WO2008103276; the contents of each of which are herein incorporated by reference in their entirety.

In one embodiment, LNP formulations described herein may comprise a polycationic composition. As a non-limiting example, the polycationic composition may be selected from formula 1-60 of US Patent Publication No. US20050222064; the content of which is herein incorporated by reference in its entirety. In another embodiment, the LNP formulations comprising a polycationic composition may be used for the delivery of the modified RNA described herein in vivo and/or in vitro.

In one embodiment, the LNP formulations described herein may additionally comprise a permeability enhancer molecule. Non-limiting permeability enhancer molecules are described in US Patent Publication No. US20050222064; the content of which is herein incorporated by reference in its entirety.

In one embodiment, the antibody compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel).

In one embodiment, the antibody compositions may be formulated in a lyophilized gel-phase liposomal composition as described in US Publication No. US2012060293, herein incorporated by reference in its entirety.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Application No. WO2013033438 or US Patent Publication No. US20130196948, the contents of each of which are herein incorporated by reference in its entirety. As a non-limiting example, the phosphate conjugates may include a compound of any one of the formulas described in International Application No. WO2013033438, herein incorporated by reference in its entirety.

The nanoparticle formulation may comprise a polymer conjugate. The polymer conjugate may be a water soluble conjugate. The polymer conjugate may have a structure as described in U.S. Patent Application No. 20130059360, the contents of which are herein incorporated by reference in its entirety. In one aspect, polymer conjugates with the polynucleotides of the present invention may be made using the methods and/or segmented polymeric reagents described in U.S. Patent Application No. 20130072709, herein incorporated by reference in its entirety. In another aspect, the polymer conjugate may have pendant side groups comprising ring moieties such as, but not limited to, the polymer conjugates described in US Patent Publication No. US20130196948, the contents of which are herein incorporated by reference in its entirety.

The nanoparticle formulations may comprise a conjugate to enhance the delivery of nanoparticles of the present invention in a subject. Further, the conjugate may inhibit phagocytic clearance of the nanoparticles in a subject. In one aspect, the conjugate may be a “self” peptide designed from the human membrane protein CD47 (e.g., the “self” particles described by Rodriguez et al (Science 2013 339, 971-975), herein incorporated by reference in its entirety). As shown by Rodriguez et al. the self peptides delayed macrophage-mediated clearance of nanoparticles which enhanced delivery of the nanoparticles. In another aspect, the conjugate may be the membrane protein CD47 (e.g., see Rodriguez et al. Science 2013 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In one embodiment, the antibody compositions of the present invention are formulated in nanoparticles which comprise a conjugate to enhance the delivery of the nanoparticles of the present invention in a subject. The conjugate may be the CD47 membrane or the conjugate may be derived from the CD47 membrane protein, such as the “self” peptide described previously. In another aspect the nanoparticle may comprise PEG and a conjugate of CD47 or a derivative thereof. In yet another aspect, the nanoparticle may comprise both the “self” peptide described above and the membrane protein CD47.

In another aspect, a “self” peptide and/or CD47 protein may be conjugated to a virus-like particle or pseudovirion, as described herein for delivery of the antibody compositions of the present invention.

In another embodiment, pharmaceutical compositions comprising the polynucleotides of the present invention and a conjugate which may have a degradable linkage. Non-limiting examples of conjugates include an aromatic moiety comprising an ionizable hydrogen atom, a spacer moiety, and a water-soluble polymer. As a non-limiting example, pharmaceutical compositions comprising a conjugate with a degradable linkage and methods for delivering such pharmaceutical compositions are described in US Patent Publication No. US20130184443, the contents of which are herein incorporated by reference in its entirety.

The nanoparticle formulations may be a carbohydrate nanoparticle comprising a carbohydrate carrier and an antibody composition. As a non-limiting example, the carbohydrate carrier may include, but is not limited to, an anhydride-modified phytoglycogen or glycogen-type material, phtoglycogen octenyl succinate, phytoglycogen beta-dextrin, anhydride-modified phytoglycogen beta-dextrin. (See e.g., International Publication No. WO2012109121; the contents of which are herein incorporated by reference in its entirety).

Nanoparticle formulations of the present invention may be coated with a surfactant or polymer in order to improve the delivery of the particle. In one embodiment, the nanoparticle may be coated with a hydrophilic coating such as, but not limited to, PEG coatings and/or coatings that have a neutral surface charge. The hydrophilic coatings may help to deliver nanoparticles with larger payloads such as, but not limited to, antibody compositions within the central nervous system. As a non-limiting example nanoparticles comprising a hydrophilic coating and methods of making such nanoparticles are described in US Patent Publication No. US20130183244, the contents of which are herein incorporated by reference in its entirety.

In one embodiment, the lipid nanoparticles of the present invention may be hydrophilic polymer particles. Non-limiting examples of hydrophilic polymer particles and methods of making hydrophilic polymer particles are described in US Patent Publication No. US20130210991, the contents of which are herein incorporated by reference in its entirety.

In another embodiment, the lipid nanoparticles of the present invention may be hydrophobic polymer particles.

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment, the internal ester linkage may be located on either side of the saturated carbon.

In one embodiment, an immune response may be elicited by delivering a lipid nanoparticle which may include a nanospecies, a polymer and an immunogen. (U.S. Publication No. 20120189700 and International Publication No. WO2012099805; each of which is herein incorporated by reference in their entirety). The polymer may encapsulate the nanospecies or partially encapsulate the nanospecies. The immunogen may be a recombinant protein, a modified RNA and/or a polynucleotide described herein. In one embodiment, the lipid nanoparticle may be formulated for use in a vaccine such as, but not limited to, against a pathogen.

Lipid nanoparticles may be engineered to alter the surface properties of particles so the lipid nanoparticles may penetrate the mucosal barrier. Mucus is located on mucosal tissue such as, but not limited to, oral (e.g., the buccal and esophageal membranes and tonsil tissue), ophthalmic, gastrointestinal (e.g., stomach, small intestine, large intestine, colon, rectum), nasal, respiratory (e.g., nasal, pharyngeal, tracheal and bronchial membranes), genital (e.g., vaginal, cervical and urethral membranes). Nanoparticles larger than 10-200 nm which are preferred for higher drug encapsulation efficiency and the ability to provide the sustained delivery of a wide array of drugs have been thought to be too large to rapidly diffuse through mucosal barriers. Mucus is continuously secreted, shed, discarded or digested and recycled so most of the trapped particles may be removed from the mucosla tissue within seconds or within a few hours. Large polymeric nanoparticles (200 nm-500 nm in diameter) which have been coated densely with a low molecular weight polyethylene glycol (PEG) diffused through mucus only 4 to 6-fold lower than the same particles diffusing in water (Lai et al. PNAS 2007 104(5):1482-487; Lai et al. Adv Drug Deliv Rev. 2009 61(2): 158-171; each of which is herein incorporated by reference in their entirety). The transport of nanoparticles may be determined using rates of permeation and/or fluorescent microscopy techniques including, but not limited to, fluorescence recovery after photobleaching (FRAP) and high resolution multiple particle tracking (MPT). As a non-limiting example, compositions which can penetrate a mucosal barrier may be made as described in U.S. Pat. No. 8,241,670 or International Patent Publication No. WO2013110028, the contents of each of which are herein incorporated by reference in its entirety.

The lipid nanoparticle engineered to penetrate mucus may comprise a polymeric material (i.e. a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyleneimines, polyisocyanates, polyacrylates, polymethacrylates, polyacrylonitriles, and polyarylates. The polymeric material may be biodegradable and/or biocompatible. Non-limiting examples of biocompatible polymers are described in International Patent Publication No. WO2013116804, the contents of which are herein incorporated by reference in its entirety. The polymeric material may additionally be irradiated. As a non-limiting example, the polymeric material may be gamma irradiated (See e.g., International App. No. WO201282165, herein incorporated by reference in its entirety). Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone. The lipid nanoparticle may be coated or associated with a co-polymer such as, but not limited to, a block co-polymer (such as a branched polyether-polyamide block copolymer described in International Publication No. WO2013012476, herein incorporated by reference in its entirety), and (poly(ethylene glycol))-(poly(propylene oxide))-(poly(ethylene glycol)) triblock copolymer (see e.g., US Publication 20120121718 and US Publication 20100003337 and U.S. Pat. No. 8,263,665; each of which is herein incorporated by reference in their entirety). The co-polymer may be a polymer that is generally regarded as safe (GRAS) and the formation of the lipid nanoparticle may be in such a way that no new chemical entities are created. For example, the lipid nanoparticle may comprise poloxamers coating PLGA nanoparticles without forming new chemical entities which are still able to rapidly penetrate human mucus (Yang et al. Angew. Chem. Int. Ed. 2011 50:2597-2600; the contents of which are herein incorporated by reference in its entirety). A non-limiting scalable method to produce nanoparticles which can penetrate human mucus is described by Xu et al. (See e.g., J Control Release 2013, 170(2):279-86; the contents of which are herein incorporated by reference in its entirety).

The vitamin of the polymer-vitamin conjugate may be vitamin E. The vitamin portion of the conjugate may be substituted with other suitable components such as, but not limited to, vitamin A, vitamin E, other vitamins, cholesterol, a hydrophobic moiety, or a hydrophobic component of other surfactants (e.g., sterol chains, fatty acids, hydrocarbon chains and alkylene oxide chains).

The lipid nanoparticle engineered to penetrate mucus may include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 dornase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle. (see e.g., US Publication 20100215580 and US Publication 20080166414 and US20130164343; each of which is herein incorporated by reference in their entirety).

In one embodiment, the mucus penetrating lipid nanoparticles may comprise at least one polynucleotide described herein. The polynucleotide may be encapsulated in the lipid nanoparticle and/or disposed on the surface of the particle. The polynucleotide may be covalently coupled to the lipid nanoparticle. Formulations of mucus penetrating lipid nanoparticles may comprise a plurality of nanoparticles. Further, the formulations may contain particles which may interact with the mucus and alter the structural and/or adhesive properties of the surrounding mucus to decrease mucoadhesion which may increase the delivery of the mucus penetrating lipid nanoparticles to the mucosal tissue.

In another embodiment, the mucus penetrating lipid nanoparticles may be a hypotonic formulation comprising a mucosal penetration enhancing coating. The formulation may be hypotonice for the epithelium to which it is being delivered. Non-limiting examples of hypotonic formulations may be found in International Patent Publication No. WO2013110028, the contents of which are herein incorporated by reference in its entirety.

In one embodiment, in order to enhance the delivery through the mucosal barrier the antibody compositions may comprise or be a hypotonic solution. Hypotonic solutions were found to increase the rate at which mucoinert particles such as, but not limited to, mucus-penetrating particles, were able to reach the vaginal epithelial surface (See e.g., Ensign et al. Biomaterials 2013 34(28):6922-9; the contents of which is herein incorporated by reference in its entirety).

In one embodiment, the antibody composition is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; all of which are incorporated herein by reference in its entirety).

In one embodiment, the antibody composition is formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. In a further embodiment, the lipid nanoparticle may be a self-assembly lipid-polymer nanoparticle (see Zhang et al., ACS Nano, 2008, 2 (8), pp 1696-1702; the contents of which are herein incorporated by reference in its entirety). As a non-limiting example, the SLN may be the SLN described in International Patent Publication No. WO2013105101, the contents of which are herein incorporated by reference in its entirety. As another non-limiting example, the SLN may be made by the methods or processes described in International Patent Publication No. WO2013105101, the contents of which are herein incorporated by reference in its entirety.

Liposomes, lipoplexes, or lipid nanoparticles may be used to improve the efficacy of polynucleotides directed protein production as these formulations may be able to increase cell transfection by the antibody composition; and/or increase the translation of encoded protein. One such example involves the use of lipid encapsulation to enable the effective systemic delivery of polyplex plasmid DNA (Heyes et al., Mol Ther. 2007 15:713-720; herein incorporated by reference in its entirety). The liposomes, lipoplexes, or lipid nanoparticles may also be used to increase the stability of the polynucleotide.

In one embodiment, the antibody compositions of the present invention can be formulated for controlled release and/or targeted delivery. As used herein, “controlled release” refers to a pharmaceutical composition or compound release profile that conforms to a particular pattern of release to elicit a therapeutic outcome. In one embodiment, the antibody compositions may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulation” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In one embodiment, the controlled release formulation may include, but is not limited to, tri-block co-polymers. As a non-limiting example, the formulation may include two different types of tri-block co-polymers (International Pub. No. WO2012131104 and WO2012131106; each of which is herein incorporated by reference in its entirety).

In another embodiment, the antibody compositions may be encapsulated into a lipid nanoparticle or a rapidly eliminated lipid nanoparticle and the lipid nanoparticles or a rapidly eliminated lipid nanoparticle may then be encapsulated into a polymer, hydrogel and/or surgical sealant described herein and/or known in the art. As a non-limiting example, the polymer, hydrogel or surgical sealant may be PLGA, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

In another embodiment, the lipid nanoparticle may be encapsulated into any polymer known in the art which may form a gel when injected into a subject. As another non-limiting example, the lipid nanoparticle may be encapsulated into a polymer matrix which may be biodegradable.

In one embodiment, the antibody composition for controlled release and/or targeted delivery may also include at least one controlled release coating. Controlled release coatings include, but are not limited to, OPADRY®, polyvinylpyrrolidone/vinyl acetate copolymer, polyvinylpyrrolidone, hydroxypropyl methylcellulose, hydroxypropyl cellulose, hydroxyethyl cellulose, EUDRAGIT RL®, EUDRAGIT RS® and cellulose derivatives such as ethylcellulose aqueous dispersions (AQUACOAT® and SURELEASE®).

In one embodiment, the antibody composition controlled release and/or targeted delivery formulation may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In one embodiment, the antibody composition controlled release and/or targeted delivery formulation comprising at least one polynucleotide may comprise at least one PEG and/or PEG related polymer derivatives as described in U.S. Pat. No. 8,404,222, herein incorporated by reference in its entirety.

In another embodiment, the antibody composition controlled release delivery formulation comprising at least one polynucleotide may be the controlled release polymer system described in US20130130348, herein incorporated by reference in its entirety.

In one embodiment, the antibody compositions of the present invention may be encapsulated in a therapeutic nanoparticle. Therapeutic nanoparticles may be formulated by methods described herein and known in the art such as, but not limited to, International Pub Nos. WO2010005740, WO2010030763, WO2010005721, WO2010005723, WO2012054923, US Pub. Nos. US20110262491, US20100104645, US20100087337, US20100068285, US20110274759, US20100068286, US20120288541, US20130123351 and US20130230567 and U.S. Pat. Nos. 8,206,747, 8,293,276, 8,318,208 and 8,318,211; the contents of each of which are herein incorporated by reference in their entirety. In another embodiment, therapeutic polymer nanoparticles may be identified by the methods described in US Pub No. US20120140790, herein incorporated by reference in its entirety.

In one embodiment, the therapeutic nanoparticle compositions may be formulated for sustained release. As used herein, “sustained release” refers to a pharmaceutical composition or compound that conforms to a release rate over a specific period of time. The period of time may include, but is not limited to, hours, days, weeks, months and years. As a non-limiting example, the sustained release nanoparticle may comprise a polymer and a therapeutic agent such as, but not limited to, the polynucleotides of the present invention (see International Pub No. 2010075072 and US Pub No. US20100216804, US20110217377 and US20120201859, each of which is herein incorporated by reference in their entirety). In another non-limiting example, the sustained release formulation may comprise agents which permit persistent bioavailability such as, but not limited to, crystals, macromolecular gels and/or particulate suspensions (see US Patent Publication No US20130150295, the contents of which is herein incorporated by reference in its entirety).

In one embodiment, the therapeutic nanoparticles compositions may be formulated to be target specific. As a non-limiting example, the therapeutic nanoparticles may include a corticosteroid (see International Pub. No. WO2011084518; herein incorporated by reference in its entirety). In one embodiment, the therapeutic nanoparticles may be formulated to be cancer specific. As a non-limiting example, the therapeutic nanoparticles may be formulated in nanoparticles described in International Pub No. WO2008121949, WO2010005726, WO2010005725, WO2011084521 and US Pub No. US20100069426, US20120004293 and US20100104655, each of which is herein incorporated by reference in their entirety.

In one embodiment, the nanoparticles of the present invention may comprise a polymeric matrix. As a non-limiting example, the nanoparticle may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof.

In one embodiment, the therapeutic nanoparticle comprises a diblock copolymer. In one embodiment, the diblock copolymer may include PEG in combination with a polymer such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester) or combinations thereof. In another embodiment, the diblock copolymer may comprise the diblock copolymers described in European Patent Publication No. the contents of which are herein incorporated by reference in its entirety. In yet another embodiment, the diblock copolymer may be a high-X diblock copolymer such as those described in International Patent Publication No. WO2013120052, the contents of which are herein incorporated by reference in its entirety.

As a non-limiting example the therapeutic nanoparticle comprises a PLGA-PEG block copolymer (see US Pub. No. US20120004293 and U.S. Pat. No. 8,236,330, each of which is herein incorporated by reference in their entirety). In another non-limiting example, the therapeutic nanoparticle is a stealth nanoparticle comprising a diblock copolymer of PEG and PLA or PEG and PLGA (see U.S. Pat. No. 8,246,968 and International Publication No. WO2012166923, the contents of each of which are herein incorporated by reference in its entirety). In yet another non-limiting example, the therapeutic nanoparticle is a stealth nanoparticle or a target-specific stealth nanoparticle as described in US Patent Publication No. US20130172406, the contents of which are herein incorporated by reference in its entirety.

In one embodiment, the therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety).

In yet another non-limiting example, the lipid nanoparticle comprises the block copolymer PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253; each of which is herein incorporated by reference in its entirety). The antibody compositions of the present invention may be formulated in lipid nanoparticles comprising the PEG-PLGA-PEG block copolymer.

In one embodiment, the therapeutic nanoparticle may comprise a multiblock copolymer (See e.g., U.S. Pat. Nos. 8,263,665 and 8,287,910 and US Patent Pub. No. US20130195987; the contents of each of which are herein incorporated by reference in its entirety).

In one embodiment, the block copolymers described herein may be included in a polyion complex comprising a non-polymeric micelle and the block copolymer. (See e.g., U.S. Pub. No. 20120076836; herein incorporated by reference in its entirety).

In one embodiment, the therapeutic nanoparticle may comprise at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the therapeutic nanoparticles may comprise at least one poly(vinyl ester) polymer. The poly(vinyl ester) polymer may be a copolymer such as a random copolymer. As a non-limiting example, the random copolymer may have a structure such as those described in International Application No. WO2013032829 or US Patent Publication No US20130121954, the contents of which are herein incorporated by reference in its entirety. In one aspect, the poly(vinyl ester) polymers may be conjugated to the polynucleotides described herein. In another aspect, the poly(vinyl ester) polymer which may be used in the present invention may be those described in, herein incorporated by reference in its entirety.

In one embodiment, the therapeutic nanoparticle may comprise at least one diblock copolymer. The diblock copolymer may be, but it not limited to, a poly(lactic) acid-poly(ethylene)glycol copolymer (see e.g., International Patent Publication No. WO2013044219; herein incorporated by reference in its entirety). As a non-limiting example, the therapeutic nanoparticle may be used to treat cancer (see International publication No. WO2013044219; herein incorporated by reference in its entirety).

In one embodiment, the therapeutic nanoparticles may comprise at least one cationic polymer described herein and/or known in the art.

In one embodiment, the therapeutic nanoparticles may comprise at least one amine-containing polymer such as, but not limited to polylysine, polyethylene imine, poly(amidoamine) dendrimers, poly(beta-amino esters) (See e.g., U.S. Pat. No. 8,287,849; herein incorporated by reference in its entirety) and combinations thereof.

In another embodiment, the nanoparticles described herein may comprise an amine cationic lipid such as those described in International Patent Application No. WO2013059496, the contents of which are herein incorporated by reference in its entirety. In one aspect the cationic lipids may have a amino-amine or an amino-amide moiety.

In one embodiment, the therapeutic nanoparticles may comprise at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

In another embodiment, the therapeutic nanoparticle may include a conjugation of at least one targeting ligand. The targeting ligand may be any ligand known in the art such as, but not limited to, a monoclonal antibody. (Kirpotin et al, Cancer Res. 2006 66:6732-6740; herein incorporated by reference in its entirety).

In one embodiment, the therapeutic nanoparticle may be formulated in an aqueous solution which may be used to target cancer (see International Pub No. WO2011084513 and US Pub No. US20110294717, each of which is herein incorporated by reference in their entirety).

In one embodiment, the therapeutic nanoparticle comprising at least one antibody composition may be formulated using the methods described by Podobinski et al in U.S. Pat. No. 8,404,799, the contents of which are herein incorporated by reference in its entirety.

In one embodiment, the antibody compositions may be encapsulated in, linked to and/or associated with synthetic nanocarriers. Synthetic nanocarriers include, but are not limited to, those described in International Pub. Nos. WO2010005740, WO2010030763, WO201213501, WO2012149252, WO2012149255, WO2012149259, WO2012149265, WO2012149268, WO2012149282, WO2012149301, WO2012149393, WO2012149405, WO2012149411, WO2012149454 and WO2013019669, and US Pub. Nos. US20110262491, US20100104645, US20100087337 and US20120244222, each of which is herein incorporated by reference in their entirety. The synthetic nanocarriers may be formulated using methods known in the art and/or described herein. As a non-limiting example, the synthetic nanocarriers may be formulated by the methods described in International Pub Nos. WO2010005740, WO2010030763 and WO201213501 and US Pub. Nos. US20110262491, US20100104645, US20100087337 and US2012024422, each of which is herein incorporated by reference in their entirety. In another embodiment, the synthetic nanocarrier formulations may be lyophilized by methods described in International Pub. No. WO2011072218 and U.S. Pat. No. 8,211,473; the content of each of which is herein incorporated by reference in their entirety. In yet another embodiment, formulations of the present invention, including, but not limited to, synthetic nanocarriers, may be lyophilized or reconstituted by the methods described in US Patent Publication No. US20130230568, the contents of which are herein incorporated by reference in its entirety.

In one embodiment, the synthetic nanocarriers may contain reactive groups to release the polynucleotides described herein (see International Pub. No. WO20120952552 and US Pub No. US20120171229, each of which is herein incorporated by reference in their entirety).

In one embodiment, the synthetic nanocarriers may contain an immunostimulatory agent to enhance the immune response from delivery of the synthetic nanocarrier. As a non-limiting example, the synthetic nanocarrier may comprise a Th1 immunostimulatory agent which may enhance a Th1-based response of the immune system (see International Pub No. WO2010123569 and US Pub. No. US20110223201, each of which is herein incorporated by reference in its entirety).

In one embodiment, the synthetic nanocarriers may be formulated for targeted release. In one embodiment, the synthetic nanocarrier is formulated to release the polynucleotides at a specified pH and/or after a desired time interval. As a non-limiting example, the synthetic nanoparticle may be formulated to release the antibody compositions after 24 hours and/or at a pH of 4.5 (see International Pub. Nos. WO2010138193 and WO2010138194 and US Pub Nos. US20110020388 and US20110027217, each of which is herein incorporated by reference in their entireties).

In one embodiment, the synthetic nanocarriers may be formulated for controlled and/or sustained release of the polynucleotides described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In one embodiment, the antibody compositions may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In one embodiment, the synthetic nanocarrier may be formulated for use as a vaccine. In one embodiment, the synthetic nanocarrier may encapsulate at least one polynucleotide which encodes at least one antibody.

In one embodiment, the synthetic nanocarrier may encapsulate at least one polynucleotide which encodes a peptide, fragment or region from a virus. As a non-limiting example, the synthetic nanocarrier may include, but is not limited to, the nanocarriers described in International Pub No. WO2012024621, WO201202629, WO2012024632 and US Pub No. US20120064110, US20120058153 and US20120058154, each of which is herein incorporated by reference in their entirety.

In one embodiment, the synthetic nanocarrier may be coupled to a polynucleotide which may be able to trigger a humoral and/or cytotoxic T lymphocyte (CTL) response (See e.g., International Publication No. WO2013019669, herein incorporated by reference in its entirety).

In one embodiment, the antibody composition may be encapsulated in, linked to and/or associated with zwitterionic lipids. Non-limiting examples of zwitterionic lipids and methods of using zwitterionic lipids are described in US Patent Publication No. US20130216607, the contents of which are herein incorporated by reference in its entirety. In one aspect, the zwitterionic lipids may be used in the liposomes and lipid nanoparticles described herein.

In one embodiment, the antibody compositions may be formulated in colloid nanocarriers as described in US Patent Publication No. US20130197100, the contents of which are herein incorporated by reference in its entirety.

In one embodiment, the nanoparticle may be optimized for oral administration. The nanoparticle may comprise at least one cationic biopolymer such as, but not limited to, chitosan or a derivative thereof. As a non-limiting example, the nanoparticle may be formulated by the methods described in U.S. Pub. No. 20120282343; herein incorporated by reference in its entirety.

In some embodiments, LNPs comprise the lipid KL52 (an amino-lipid disclosed in U.S. Application Publication No. 2012/0295832 expressly incorporated herein by reference in its entirety). Activity and/or safety (as measured by examining one or more of ALT/AST, white blood cell count and cytokine induction) of LNP administration may be improved by incorporation of such lipids. LNPs comprising KL52 may be administered intravenously and/or in one or more doses. In some embodiments, administration of LNPs comprising KL52 results in equal or improved mRNA and/or protein expression as compared to LNPs comprising MC3.

In some embodiments, antibody compositions may be delivered using smaller LNPs. Such particles may comprise a diameter from below 0.1 um up to 100 nm such as, but not limited to, less than 0.1 um, less than 1.0 um, less than 5 um, less than 10 um, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, less than 975 um,

In another embodiment, antibody compositions may be delivered using smaller LNPs which may comprise a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm.

In some embodiments, such LNPs are synthesized using methods comprising microfluidic mixers. Exemplary microfluidic mixers may include, but are not limited to a slit interdigitial micromixer including, but not limited to those manufactured by Microinnova (Allerheiligen bei Wildon, Austria) and/or a staggered herringbone micromixer (SHM) (Zhigaltsev, I. V. et al., Bottom-up design and synthesis of limit size lipid nanoparticle systems with aqueous and triglyceride cores using millisecond microfluidic mixing have been published (Langmuir. 2012. 28:3633-40; Belliveau, N. M. et al., Microfluidic synthesis of highly potent limit-size lipid nanoparticles for in vivo delivery of siRNA. Molecular Therapy-Nucleic Acids. 2012. 1:e37; Chen, D. et al., Rapid discovery of potent siRNA-containing lipid nanoparticles enabled by controlled microfluidic formulation. J Am Chem Soc. 2012. 134(16):6948-51; each of which is herein incorporated by reference in its entirety). In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Application Publication Nos. 2004/0262223 and 2012/0276209, each of which is expressly incorporated herein by reference in their entirety.

In one embodiment, the antibody compositions of the present invention may be formulated in lipid nanoparticles created using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany).

In one embodiment, the antibody compositions of the present invention may be formulated in lipid nanoparticles created using microfluidic technology (see Whitesides, George M. The Origins and the Future of Microfluidics. Nature, 2006 442: 368-373; and Abraham et al. Chaotic Mixer for Microchannels. Science, 2002 295: 647-651; each of which is herein incorporated by reference in its entirety). As a non-limiting example, controlled microfluidic formulation includes a passive method for mixing streams of steady pressure-driven flows in micro channels at a low Reynolds number (See e.g., Abraham et al. Chaotic Mixer for Microchannels. Science, 2002 295: 647-651; which is herein incorporated by reference in its entirety).

In one embodiment, the antibody compositions of the present invention may be formulated in lipid nanoparticles created using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In one embodiment, the antibody compositions of the invention may be formulated for delivery using the drug encapsulating microspheres described in International Patent Publication No. WO2013063468 or U.S. Pat. No. 8,440,614, each of which is herein incorporated by reference in its entirety. The microspheres may comprise a compound of the formula (I), (II), (III), (IV), (V) or (VI) as described in International Patent Application No. WO2013063468, the contents of which are herein incorporated by reference in its entirety. In another aspect, the amino acid, peptide, polypeptide, lipids (APPL) are useful in delivering the antibody compositions of the invention to cells (see International Patent Publication No. WO2013063468, herein incorporated by reference in its entirety).

In one embodiment, the antibody compositions of the invention may be formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the lipid nanoparticles may have a diameter from about 10 to 500 nm.

In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In one aspect, the lipid nanoparticle may be a limit size lipid nanoparticle described in International Patent Publication No. WO2013059922, the contents of which are herein incorporated by reference in its entirety. The limit size lipid nanoparticle may comprise a lipid bilayer surrounding an aqueous core or a hydrophobic core; where the lipid bilayer may comprise a phospholipid such as, but not limited to, diacylphosphatidylcholine, a diacylphosphatidylethanolamine, a ceramide, a sphingomyelin, a dihydrosphingomyelin, a cephalin, a cerebroside, a C8-C20 fatty acid diacylphophatidylcholine, and 1-palmitoyl-2-oleoyl phosphatidylcholine (POPC). In another aspect the limit size lipid nanoparticle may comprise a polyethylene glycol-lipid such as, but not limited to, DLPE-PEG, DMPE-PEG, DPPC-PEG and DSPE-PEG.

In one embodiment, the antibody compositions may be delivered, localized and/or concentrated in a specific location using the delivery methods described in International Patent Publication No. WO2013063530, the contents of which are herein incorporated by reference in its entirety. As a non-limiting example, a subject may be administered an empty polymeric particle prior to, simultaneously with or after delivering the antibody compositions to the subject. The empty polymeric particle undergoes a change in volume once in contact with the subject and becomes lodged, embedded, immobilized or entrapped at a specific location in the subject.

In one embodiment, the antibody compositions may be formulated in an active substance release system (See e.g., US Patent Publication No. US20130102545, herein incorporated by reference in its entirety). The active substance release system may comprise 1) at least one nanoparticle bonded to an oligonucleotide inhibitor strand which is hybridized with a catalytically active nucleic acid and 2) a compound bonded to at least one substrate molecule bonded to a therapeutically active substance (e.g., polynucleotides described herein), where the therapeutically active substance is released by the cleavage of the substrate molecule by the catalytically active nucleic acid.

In one embodiment, the antibody compositions may be formulated in a nanoparticle comprising an inner core comprising a non-cellular material and an outer surface comprising a cellular membrane. The cellular membrane may be derived from a cell or a membrane derived from a virus. As a non-limiting example, the nanoparticle may be made by the methods described in International Patent Publication No. WO2013052167, herein incorporated by reference in its entirety. As another non-limiting example, the nanoparticle described in International Patent Publication No. WO2013052167, the contents of which are herein incorporated by reference in its entirety, may be used to deliver the antibody compositions described herein.

In one embodiment, the antibody compositions may be formulated in porous nanoparticle-supported lipid bilayers (protocells). Protocells are described in International Patent Publication No. WO2013056132, the contents of which are herein incorporated by reference in its entirety.

In one embodiment, the antibody compositions described herein may be formulated in polymeric nanoparticles as described in or made by the methods described in U.S. Pat. Nos. 8,420,123 and 8,518,963 and European Patent No. EP2073848B1, the contents of each of which are herein incorporated by reference in their entirety. As a non-limiting example, the polymeric nanoparticle may have a high glass transition temperature such as the nanoparticles described in or nanoparticles made by the methods described in U.S. Pat. No. 8,518,963, the contents of which are herein incorporated by reference in its entirety. As another non-limiting example, the polymer nanoparticle for oral, parenteral and topical formulations may be made by the methods described in European Patent No. EP2073848B1, the contents of which are herein incorporated by reference in its entirety.

In another embodiment, the antibody compositions described herein may be formulated in nanoparticles used in imaging. The nanoparticles may be liposome nanoparticles such as those described in US Patent Publication No US20130129636, herein incorporated by reference in its entirety. As a non-limiting example, the liposome may comprise gadolinium(III)2-{4,7-bis-carboxymethyl-10-[(N,N-distearylamidomethyl-N-amido-methyl]-1,4,7,10-tetra-azacyclododec-1-yl}-acetic acid and a neutral, fully saturated phospholipid component (see e.g., US Patent Publication No US20130129636, the contents of which is herein incorporated by reference in its entirety).

In one embodiment, the nanoparticles which may be used in the present invention are formed by the methods described in U.S. Patent Application No. US20130130348, the contents of which are herein incorporated by reference in its entirety.

The nanoparticles of the present invention may further include nutrients such as, but not limited to, those which deficiencies can lead to health hazards from anemia to neural tube defects (see e.g, the nanoparticles described in International Patent Publication No WO2013072929, the contents of which is herein incorporated by reference in its entirety). As a non-limiting example, the nutrient may be iron in the form of ferrous, ferric salts or elemental iron, iodine, folic acid, vitamins or micronutrients.

In one embodiment, the antibody compositions of the present invention may be formulated in a swellable nanoparticle. The swellable nanoparticle may be, but is not limited to, those described in U.S. Pat. No. 8,440,231, the contents of which is herein incorporated by reference in its entirety. As a non-limiting embodiment, the swellable nanoparticle may be used for delivery of the antibody compositions of the present invention to the pulmonary system (see e.g., U.S. Pat. No. 8,440,231, the contents of which is herein incorporated by reference in its entirety).

The antibody compositions of the present invention may be formulated in polyanhydride nanoparticles such as, but not limited to, those described in U.S. Pat. No. 8,449,916, the contents of which is herein incorporated by reference in its entirety.

The nanoparticles and microparticles of the present invention may be geometrically engineered to modulate macrophage and/or the immune response. In one aspect, the geometrically engineered particles may have varied shapes, sizes and/or surface charges in order to incorporated the polynucleotides of the present invention for targeted delivery such as, but not limited to, pulmonary delivery (see e.g., International Publication No WO2013082111, the contents of which is herein incorporated by reference in its entirety). Other physical features the geometrically engineering particles may have include, but are not limited to, fenestrations, angled arms, asymmetry and surface roughness, charge which can alter the interactions with cells and tissues. As a non-limiting example, nanoparticles of the present invention may be made by the methods described in International Publication No WO2013082111, the contents of which are herein incorporated by reference in its entirety.

In one embodiment, the nanoparticles of the present invention may be water soluble nanoparticles such as, but not limited to, those described in International Publication No. WO2013090601, the contents of which is herein incorporated by reference in its entirety. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In one embodiment the nanoparticles of the present invention may be developed by the methods described in US Patent Publication No. US20130172406, the contents of which are herein incorporated by reference in its entirety.

In one embodiment, the nanoparticles of the present invention are stealth nanoparticles or target-specific stealth nanoparticles such as, but not limited to, those described in US Patent Publication No. US20130172406; the contents of which is herein incorporated by reference in its entirety. The nanoparticles of the present invention may be made by the methods described in US Patent Publication No. US20130172406, the contents of which are herein incorporated by reference in its entirety.

In another embodiment, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In one embodiment, the nanoparticle may be a nanoparticle-nucleic acid hybrid structure having a high density nucleic acid layer. As a non-limiting example, the nanoparticle-nucleic acid hybrid structure may made by the methods described in US Patent Publication No. US20130171646, the contents of which are herein incorporated by reference in its entirety. The nanoparticle may comprise a nucleic acid such as, but not limited to, polynucleotides described herein and/or known in the art.

At least one of the nanoparticles of the present invention may be embedded in in the core a nanostructure or coated with a low density porous 3-D structure or coating which is capable of carrying or associating with at least one payload within or on the surface of the nanostructure. Non-limiting examples of the nanostructures comprising at least one nanoparticle are described in International Patent Publication No. WO2013123523, the contents of which are herein incorporated by reference in its entirety.

### Polymers, Biodegradable Nanoparticles, and Core-Shell Nanoparticles

The antibody compositions of the invention can be formulated using natural and/or synthetic polymers. Non-limiting examples of polymers which may be used for delivery include, but are not limited to, DYNAMIC POLYCONJUGATE® (Arrowhead Research Corp., Pasadena, Calif.) formulations from MIRUS® Bio (Madison, Wis.) and Roche Madison (Madison, Wis.), PHASERX™ polymer formulations such as, without limitation, SMARTT POLYMER TECHNOLOGY™ (PHASERX®, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers. RONDEL™ (RNAi/Oligonucleotide Nanoparticle Delivery) polymers (Arrowhead Research Corporation, Pasadena, Calif.) and pH responsive co-block polymers such as, but not limited to, PHASERX® (Seattle, Wash.).

A non-limiting example of chitosan formulation includes a core of positively charged chitosan and an outer portion of negatively charged substrate (U.S. Pub. No. 20120258176; herein incorporated by reference in its entirety). Chitosan includes, but is not limited to N-trimethyl chitosan, mono-N-carboxymethyl chitosan (MCC), N-palmitoyl chitosan (NPCS), EDTA-chitosan, low molecular weight chitosan, chitosan derivatives, or combinations thereof.

In one embodiment, the polymers used in the present invention have undergone processing to reduce and/or inhibit the attachment of unwanted substances such as, but not limited to, bacteria, to the surface of the polymer. The polymer may be processed by methods known and/or described in the art and/or described in International Pub. No. WO2012150467, herein incorporated by reference in its entirety.

A non-limiting example of PLGA formulations include, but are not limited to, PLGA injectable depots (e.g., ELIGARD® which is formed by dissolving PLGA in 66% N-methyl-2-pyrrolidone (NMP) and the remainder being aqueous solvent and leuprolide. Once injected, the PLGA and leuprolide peptide precipitates into the subcutaneous space).

Many of these polymer approaches have demonstrated efficacy in delivering oligonucleotides in vivo into the cell cytoplasm (reviewed in deFougerolles Hum Gene Ther. 2008 19:125-132; herein incorporated by reference in its entirety). Two polymer approaches that have yielded robust in vivo delivery of nucleic acids, in this case with small interfering RNA (siRNA), are dynamic polyconjugates and cyclodextrin-based nanoparticles (see e.g., US Patent Publication No. US20130156721, herein incorporated by reference in its entirety). The first of these delivery approaches uses dynamic polyconjugates and has been shown in vivo in mice to effectively deliver siRNA and silence endogenous target mRNA in hepatocytes (Rozema et al., Proc Natl Acad Sci USA. 2007 104:12982-12887; herein incorporated by reference in its entirety). This particular approach is a multicomponent polymer system whose key features include a membrane-active polymer to which nucleic acid, in this case siRNA, is covalently coupled via a disulfide bond and where both PEG (for charge masking) and N-acetylgalactosamine (for hepatocyte targeting) groups are linked via pH-sensitive bonds (Rozema et al., Proc Natl Acad Sci USA. 2007 104:12982-12887; herein incorporated by reference in its entirety). On binding to the hepatocyte and entry into the endosome, the polymer complex disassembles in the low-pH environment, with the polymer exposing its positive charge, leading to endosomal escape and cytoplasmic release of the siRNA from the polymer. Through replacement of the N-acetylgalactosamine group with a mannose group, it was shown one could alter targeting from asialoglycoprotein receptor-expressing hepatocytes to sinusoidal endothelium and Kupffer cells. Another polymer approach involves using transferrin-targeted cyclodextrin-containing polycation nanoparticles. These nanoparticles have demonstrated targeted silencing of the EWS-FLI1 gene product in transferrin receptor-expressing Ewing's sarcoma tumor cells (Hu-Lieskovan et al., Cancer Res. 2005 65: 8984-8982; herein incorporated by reference in its entirety) and siRNA formulated in these nanoparticles was well tolerated in non-human primates (Heidel et al., Proc Natl Acad Sci USA 2007 104:5715-21; herein incorporated by reference in its entirety). Both of these delivery strategies incorporate rational approaches using both targeted delivery and endosomal escape mechanisms.

The polymer formulation can permit the sustained or delayed release of polynucleotides (e.g., following intramuscular or subcutaneous injection). The altered release profile for the polynucleotide can result in, for example, translation of an encoded protein over an extended period of time. The polymer formulation may also be used to increase the stability of the polynucleotide. Biodegradable polymers have been previously used to protect nucleic acids other than polynucleotide from degradation and been shown to result in sustained release of payloads in vivo (Rozema et al., Proc Natl Acad Sci USA. 2007 104:12982-12887; Sullivan et al., Expert Opin Drug Deliv. 2010 7:1433-1446; Convertine et al., Biomacromolecules. 2010 Oct. 1; Chu et al., Acc Chem Res. 2012 Jan. 13; Manganiello et al., Biomaterials. 2012 33:2301-2309; Benoit et al., Biomacromolecules. 2011 12:2708-2714; Singha et al., Nucleic Acid Ther. 2011 2:133-147; deFougerolles Hum Gene Ther. 2008 19:125-132; Schaffert and Wagner, Gene Ther. 2008 16:1131-1138; Chaturvedi et al., Expert Opin Drug Deliv. 2011 8:1455-1468; Davis, Mol Pharm. 2009 6:659-668; Davis, Nature 2010 464:1067-1070; each of which is herein incorporated by reference in its entirety).

In one embodiment, the antibody compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.), TISSELL® (Baxter International, Inc Deerfield, Ill.), PEG-based sealants, and COSEAL® (Baxter International, Inc Deerfield, Ill.).

As a non-limiting example, antibody compositions may be formulated in PLGA microspheres by preparing the PLGA microspheres with tunable release rates (e.g., days and weeks) and encapsulating the modified mRNA in the PLGA microspheres while maintaining the integrity of the modified mRNA during the encapsulation process. EVAc are non-biodegradable, biocompatible polymers which are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. PEG-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic ineraction to provide a stabilizing effect.

Polymer formulations can also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070; each of which is herein incorporated by reference in its entirety).

The antibody compositions of the invention may be formulated with or in a polymeric compound. The polymer may include at least one polymer such as, but not limited to, polyethenes, polyethylene glycol (PEG), poly(l-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

As a non-limiting example, the antibody compositions of the invention may be formulated with the polymeric compound of PEG grafted with PLL as described in U.S. Pat. No. 6,177,274; herein incorporated by reference in its entirety. The formulation may be used for transfecting cells in vitro or for in vivo delivery of polynucleotide. In another example, the polynucleotide may be suspended in a solution or medium with a cationic polymer, in a dry pharmaceutical composition or in a solution that is capable of being dried as described in U.S. Pub. Nos. 20090042829 and 20090042825; each of which are herein incorporated by reference in their entireties.

As another non-limiting example the antibody compositions of the invention may be formulated with a PLGA-PEG block copolymer (see US Pub. No. US20120004293 and U.S. Pat. No. 8,236,330, herein incorporated by reference in their entireties) or PLGA-PEG-PLGA block copolymers (See U.S. Pat. No. 6,004,573, herein incorporated by reference in its entirety). As a non-limiting example, the antibody compositions of the invention may be formulated with a diblock copolymer of PEG and PLA or PEG and PLGA (see U.S. Pat. No. 8,246,968, herein incorporated by reference in its entirety).

A polyamine derivative may be used to deliver nucleic acids or to treat and/or prevent a disease or to be included in an implantable or injectable device (U.S. Pub. No. 20100260817 (now U.S. Pat. No. 8,460,696) the contents of each of which is herein incorporated by reference in its entirety). As a non-limiting example, a pharmaceutical composition may include the antibody composition and the polyamine derivative described in U.S. Pub. No. 20100260817 (now U.S. Pat. No. 8,460,696; the contents of which are incorporated herein by reference in its entirety. As a non-limiting example the antibody compositions of the present invention may be delivered using a polyaminde polymer such as, but not limited to, a polymer comprising a 1,3-dipolar addition polymer prepared by combining a carbohydrate diazide monomer with a dilkyne unite comprising oligoamines (U.S. Pat. No. 8,236,280; herein incorporated by reference in its entirety).

The antibody compositions of the invention may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

In one embodiment, the antibody compositions of the present invention may be formulated with at least one polymer and/or derivatives thereof described in International Publication Nos. WO2011115862, WO2012082574 and WO2012068187 and U.S. Pub. No. 20120283427, each of which are herein incorporated by reference in their entireties.

In another embodiment, the antibody compositions of the present invention may be formulated with a polymer of formula Z as described in WO2011115862, herein incorporated by reference in its entirety. In yet another embodiment, the antibody compositions may be formulated with a polymer of formula Z, Z′ or Z″ as described in International Pub. Nos. WO2012082574 or WO2012068187 and U.S. Pub. No. 2012028342, each of which are herein incorporated by reference in their entireties. The polymers formulated with the modified RNA of the present invention may be synthesized by the methods described in International Pub. Nos. WO2012082574 or WO2012068187, each of which are herein incorporated by reference in their entireties.

The antibody compositions of the invention may be formulated with at least one acrylic polymer. Acrylic polymers include but are not limited to, acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof.

Formulations of antibody compositions of the invention may include at least one amine-containing polymer such as, but not limited to polylysine, polyethylene imine, poly(amidoamine) dendrimers, poly(amine-co-esters) or combinations thereof. As a non-limiting example, the poly(amine-co-esters) may be the polymers described in and/or made by the methods described in International Publication No WO2013082529, the contents of which are herein incorporated by reference in its entirety.

For example, the antibody compositions of the invention may be formulated in a pharmaceutical compound including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer may be made by methods known in the art and/or described in U.S. Pat. No. 6,696,038, U.S. App. Nos. 20030073619 and 20040142474 each of which is herein incorporated by reference in their entireties. The poly(alkylene imine) may be made using methods known in the art and/or as described in U.S. Pub. No. 20100004315, herein incorporated by reference in its entirety. The biodegradable polymer, biodegradable block copolymer, the biodegradable random copolymer, biodegradable polyester block copolymer, biodegradable polyester polymer, or biodegradable polyester random copolymer may be made using methods known in the art and/or as described in U.S. Pat. Nos. 6,517,869 and 6,267,987, the contents of which are each incorporated herein by reference in their entirety. The linear biodegradable copolymer may be made using methods known in the art and/or as described in U.S. Pat. No. 6,652,886. The PAGA polymer may be made using methods known in the art and/or as described in U.S. Pat. No. 6,217,912 herein incorporated by reference in its entirety. The PAGA polymer may be copolymerized to form a copolymer or block copolymer with polymers such as but not limited to, poly-L-lysine, polyargine, polyornithine, histones, avidin, protamines, polylactides and poly(lactide-co-glycolides). The biodegradable cross-linked cationic multi-block copolymers may be made my methods known in the art and/or as described in U.S. Pat. Nos. 8,057,821, 8,444,992 or U.S. Pub. No. 2012009145 each of which are herein incorporated by reference in their entireties. For example, the multi-block copolymers may be synthesized using linear polyethyleneimine (LPEI) blocks which have distinct patterns as compared to branched polyethyleneimines. Further, the composition or pharmaceutical composition may be made by the methods known in the art, described herein, or as described in U.S. Pub. No. 20100004315 or U.S. Pat. Nos. 6,267,987 and 6,217,912 each of which are herein incorporated by reference in their entireties.

The antibody compositions of the invention may be formulated with at least one degradable polyester which may contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters may include a PEG conjugation to form a PEGylated polymer.

The antibody compositions of the invention may be formulated with at least one crosslinkable polyester. Crosslinkable polyesters include those known in the art and described in US Pub. No. 20120269761, the contents of which are herein incorporated by reference in its entirety.

The antibody compositions of the invention may be formulated in or with at least one cyclodextrin polymer. Cyclodextrin polymers and methods of making cyclodextrin polymers include those known in the art and described in US Pub. No. 20130184453, the contents of which are herein incorporated by reference in its entirety.

In one embodiment, the antibody compositions of the invention may be formulated in or with at least one crosslinked cation-binding polymers. Crosslinked cation-binding polymers and methods of making crosslinked cation-binding polymers include those known in the art and described in International Patent Publication No. WO2013106072, WO2013106073 and WO2013106086, the contents of each of which are herein incorporated by reference in its entirety.

In one embodiment, the antibody compositions of the invention may be formulated in or with at least one branched polymer. Branched polymers and methods of making branched polymers include those known in the art and described in International Patent Publication No. WO2013113071, the contents of each of which are herein incorporated by reference in its entirety.

In one embodiment, the antibody compositions of the invention may be formulated in or with at least PEGylated albumin polymer. PEGylated albumin polymer and methods of making PEGylated albumin polymer include those known in the art and described in US Patent Publication No. US20130231287, the contents of each of which are herein incorporated by reference in its entirety.

In one embodiment, the polymers described herein may be conjugated to a lipid-terminating PEG. As a non-limiting example, PLGA may be conjugated to a lipid-terminating PEG forming PLGA-DSPE-PEG. As another non-limiting example, PEG conjugates for use with the present invention are described in International Publication No. WO2008103276, herein incorporated by reference in its entirety. The polymers may be conjugated using a ligand conjugate such as, but not limited to, the conjugates described in U.S. Pat. No. 8,273,363, herein incorporated by reference in its entirety.

In one embodiment, the antibody compositions disclosed herein may be mixed with the PEGs or the sodium phosphate/sodium carbonate solution prior to administration.

In another embodiment, polynucleotides encoding a protein of interest may be mixed with the PEGs and also mixed with the sodium phosphate/sodium carbonate solution.

In yet another embodiment, polynucleotides encoding a protein of interest may be mixed with the PEGs and polynucleotides encoding a second protein of interest may be mixed with the sodium phosphate/sodium carbonate solution.

In one embodiment, the antibody compositions described herein may be conjugated with another compound. Non-limiting examples of conjugates are described in U.S. Pat. Nos. 7,964,578 and 7,833,992, each of which are herein incorporated by reference in their entireties. In another embodiment, modified RNA of the present invention may be conjugated with conjugates of formula 1-122 as described in U.S. Pat. Nos. 7,964,578 and 7,833,992, each of which are herein incorporated by reference in their entireties. The antibody compositions described herein may be conjugated with a metal such as, but not limited to, gold. (See e.g., Giljohann et al. Journ. Amer. Chem. Soc. 2009 131(6): 2072-2073; herein incorporated by reference in its entirety). In another embodiment, the antibody compositions described herein may be conjugated and/or encapsulated in gold-nanoparticles. (International Pub. No. WO201216269 and U.S. Pub. No. 20120302940 and US20130177523; the contents of each of which is herein incorporated by reference in its entirety).

As described in U.S. Pub. No. 20100004313, herein incorporated by reference in its entirety, a gene delivery composition may include a nucleotide sequence and a poloxamer. For example, the antibody compositions of the present invention may be used in a gene delivery composition with the poloxamer described in U.S. Pub. No. 20100004313.

In one embodiment, the polymer formulation of the present invention may be stabilized by contacting the polymer formulation, which may include a cationic carrier, with a cationic lipopolymer which may be covalently linked to cholesterol and polyethylene glycol groups. The polymer formulation may be contacted with a cationic lipopolymer using the methods described in U.S. Pub. No. 20090042829 herein incorporated by reference in its entirety. The cationic carrier may include, but is not limited to, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N—(N′,N′-Dimethylaminoethane)-carbamoyl]Cholesterol Hydrochloride (DC-Cholesterol HCl) diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride DODAC) and combinations thereof. As a non-limiting example, the antibody compositions may be formulated with a cationic lipopolymer such as those described in U.S. Patent Application No. 20130065942, herein incorporated by reference in its entirety.

The antibody compositions of the invention may be formulated in a polyplex of one or more polymers (See e.g., U.S. Pat. No. 8,501,478, U.S. Pub. No. 20120237565 and 20120270927 and 20130149783 and International Patent Pub. No. WO2013090861; the contents of each of which are herein incorporated by reference in its entirety). As a non-limiting example, the polyplex may be formed using the noval alpha-aminoamidine polymers described in International Publication No. WO2013090861, the contents of which are herein incorporated by reference in its entirety. As another non-limiting example, the polyplex may be formed using the click polymers described in U.S. Pat. No. 8,501,478, the contents of which is herein incorporated by reference in its entirety.

In one embodiment, the polyplex comprises two or more cationic polymers. The catioinic polymer may comprise a poly(ethylene imine) (PEI) such as linear PEI. In another embodiment, the polyplex comprises p(TETA/CBA) its PEGylated analog p(TETA/CBA)-g-PEG2k and mixtures thereof (see e.g., US Patent Publication No. US20130149783, the contents of which are herein incorporated by reference in its entirety.

The antibody compositions of the invention can also be formulated as a nanoparticle using a combination of polymers, lipids, and/or other biodegradable agents, such as, but not limited to, calcium phosphate. Components may be combined in a core-shell, hybrid, and/or layer-by-layer architecture, to allow for fine-tuning of the nanoparticle so to delivery of the antibody composition, may be enhanced (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87; herein incorporated by reference in its entirety). As a non-limiting example, the nanoparticle may comprise a plurality of polymers such as, but not limited to hydrophilic-hydrophobic polymers (e.g., PEG-PLGA), hydrophobic polymers (e.g., PEG) and/or hydrophilic polymers (International Pub. No. WO20120225129; the contents of which is herein incorporated by reference in its entirety).

As another non-limiting example the nanoparticle comprising hydrophilic polymers for the antibody compositions may be those described in or made by the methods described in International Patent Publication No. WO2013119936, the contents of which are herein incorporated by reference in its entirety.

In one embodiment, the biodegradable polymers which may be used in the present invention are poly(ether-anhydride) block copolymers. As a non-limiting example, the biodegradable polymers used herein may be a block copolymer as described in International Patent Publication No WO2006063249, herein incorporated by reference in its entirety, or made by the methods described in International Patent Publication No WO2006063249, herein incorporated by reference in its entirety.

In another embodiment, the biodegradable polymers which may be used in the present invention are alkyl and cycloalkyl terminated biodegradable lipids. As a non-limiting example, the alkyl and cycloalkyl terminated biodegradable lipids may be those described in International Publication No. WO2013086322 and/or made by the methods described in International Publication No. WO2013086322; the contents of which are herein incorporated by reference in its entirety.

In yet another embodiment, the biodegradable polymers which may be used in the present invention are cationic lipids having one or more biodegradable group located in a lipid moiety. As a non-limiting example, the biodegradable lipids may be those described in US Patent Publication No. US20130195920, the contents of which are herein incorporated by reference in its entirety.

Biodegradable calcium phosphate nanoparticles in combination with lipids and/or polymers have been shown to deliver polynucleotides in vivo. In one embodiment, a lipid coated calcium phosphate nanoparticle, which may also contain a targeting ligand such as anisamide, may be used to deliver the antibody compositions of the present invention. For example, to effectively deliver siRNA in a mouse metastatic lung model a lipid coated calcium phosphate nanoparticle was used (Li et al., J Contr Rel. 2010 142: 416-421; Li et al., J Contr Rel. 2012 158:108-114; Yang et al., Mol Ther. 2012 20:609-615; herein incorporated by reference in its entirety). This delivery system combines both a targeted nanoparticle and a component to enhance the endosomal escape, calcium phosphate, in order to improve delivery of the siRNA.

In one embodiment, calcium phosphate with a PEG-polyanion block copolymer may be used to delivery antibody compositions (Kazikawa et al., J Contr Rel. 2004 97:345-356; Kazikawa et al., J Contr Rel. 2006 111:368-370; the contents of each of which are herein incorporated by reference in its entirety).

In one embodiment, a PEG-charge-conversional polymer (Pitella et al., Biomaterials. 2011 32:3106-3114; the contents of which are herein incorporated by reference in its entirety) may be used to form a nanoparticle to deliver the antibody compositions of the present invention. The PEG-charge-conversional polymer may improve upon the PEG-polyanion block copolymers by being cleaved into a polycation at acidic pH, thus enhancing endosomal escape.

In one embodiment, a polymer used in the present invention may be a pentablock polymer such as, but not limited to, the pentablock polymers described in International Patent Publication No. WO2013055331, herein incorporated by reference in its entirety. As a non-limiting example, the pentablock polymer comprises PGA-PCL-PEG-PCL-PGA, wherein PEG is polyethylene glycol, PCL is poly(E-caprolactone), PGA is poly(glycolic acid), and PLA is poly(lactic acid). As another non-limiting example, the pentablock polymer comprises PEG-PCL-PLA-PCL-PEG, wherein PEG is polyethylene glycol, PCL is poly(E-caprolactone), PGA is poly(glycolic acid), and PLA is poly(lactic acid).

In one embodiment, a polymer which may be used in the present invention comprises at least one diepoxide and at least one aminoglycoside (See e.g., International Patent Publication No. WO2013055971, the contents of which are herein incorporated by reference in its entirety). The diepoxide may be selected from, but is not limited to, 1,4 butanediol diglycidyl ether (1,4 B), 1,4-cyclohexanedimethanol diglycidyl ether (1,4 C), 4-vinylcyclohexene diepoxide (4VCD), ethyleneglycol diglycidyl ether (EDGE), glycerol diglycidyl ether (GDE), neopentylglycol diglycidyl ether (NPDGE), poly(ethyleneglycol) diglycidyl ether (PEGDE), poly(propyleneglycol) diglycidyl ether (PPGDE) and resorcinol diglycidyl ether (RDE). The aminoglycoside may be selected from, but is not limited to, streptomycin, neomycin, framycetin, paromomycin, ribostamycin, kanamycin, amikacin, arbekacin, bekanamycin, dibekacin, tobramycin, spectinomycin, hygromycin, gentamicin, netilmicin, sisomicin, isepamicin, verdamicin, astromicin, and apramycin. As a non-limiting example, the polymers may be made by the methods described in International Patent Publication No. WO2013055971, the contents of which are herein incorporated by reference in its entirety. As another non-limiting example, compositions comprising any of the polymers comprising at least one least one diepoxide and at least one aminoglycoside may be made by the methods described in International Patent Publication No. WO2013055971, the contents of which are herein incorporated by reference in its entirety.

In one embodiment, a polymer which may be used in the present invention may be a cross-linked polymer. As a non-limiting example, the cross-linked polymers may be used to form a particle as described in U.S. Pat. No. 8,414,927, the contents of which are herein incorporated by reference in its entirety. As another non-limiting example, the cross-linked polymer may be obtained by the methods described in US Patent Publication No. US20130172600, the contents of which are herein incorporated by reference in its entirety.

In another embodiment, a polymer which may be used in the present invention may be a cross-linked polymer such as those described in U.S. Pat. No. 8,461,132, the contents of which are herein incorporated by reference in its entirety. As a non-limiting example, the cross-linked polymer may be used in a therapeutic composition for the treatment of a body tissue. The therapeutic composition may be administered to damaged tissue using various methods known in the art and/or described herein such as injection or catheterization.

In one embodiment, a polymer which may be used in the present invention may be a di-alphatic substituted pegylated lipid such as, but not limited to, those described in International Patent Publication No. WO2013049328, the contents of which are herein incorporated by reference in its entirety.

In one embodiment, a block copolymer is PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253; each of which is herein incorporated by reference in its entirety) may be used in the present invention. The present invention may be formulated with PEG-PLGA-PEG for administration such as, but not limited to, intramuscular and subcutaneous administration.

In another embodiment, the PEG-PLGA-PEG block copolymer is used in the present invention to develop a biodegradable sustained release system. In one aspect, the antibody compositions of the present invention are mixed with the block copolymer prior to administration. In another aspect, the antibody compositions of the present invention are co-administered with the block copolymer.

In one embodiment, the polymer used in the present invention may be a multi-functional polymer derivative such as, but not limited to, a multi-functional N-maleimidyl polymer derivatives as described in U.S. Pat. No. 8,454,946, the contents of which are herein incorporated by reference in its entirety.

The use of core-shell nanoparticles has additionally focused on a high-throughput approach to synthesize cationic cross-linked nanogel cores and various shells (Siegwart et al., Proc Natl Acad Sci USA. 2011 108:12996-13001; the contents of which are herein incorporated by reference in its entirety). The complexation, delivery, and internalization of the polymeric nanoparticles can be precisely controlled by altering the chemical composition in both the core and shell components of the nanoparticle. For example, the core-shell nanoparticles may efficiently deliver siRNA to mouse hepatocytes after they covalently attach cholesterol to the nanoparticle.

In one embodiment, a hollow lipid core comprising a middle PLGA layer and an outer neutral lipid layer containing PEG may be used to delivery of the antibody compositions of the present invention. As a non-limiting example, in mice bearing a luciferase-expressing tumor, it was determined that the lipid-polymer-lipid hybrid nanoparticle significantly suppressed luciferase expression, as compared to a conventional lipoplex (Shi et al, Angew Chem Int Ed. 2011 50:7027-7031; herein incorporated by reference in its entirety).

In one embodiment, the lipid nanoparticles may comprise a core of the antibody compositions disclosed herein and a polymer shell. The polymer shell may be any of the polymers described herein and are known in the art. In an additional embodiment, the polymer shell may be used to protect the polynucleotides in the core.

Core-shell nanoparticles for use with the antibody compositions of the present invention are described and may be formed by the methods described in U.S. Pat. No. 8,313,777 or International Patent Publication No. WO2013124867, the contents of each of which are herein incorporated by reference in their entirety.

In one embodiment, the polymer used with the formulations described herein may be a modified polymer (such as, but not limited to, a modified polyacetal) as described in International Publication No. WO2011120053, the contents of which are herein incorporated by reference in its entirety.

In one embodiment, the formulation may be a polymeric carrier cargo complex comprising a polymeric carrier and at least one nucleic acid molecule. Non-limiting examples of polymeric carrier cargo complexes are described in International Patent Publications Nos. WO2013113326, WO2013113501, WO2013113325, WO2013113502 and WO2013113736 and European Patent Publication No. EP2623121, the contents of each of which are herein incorporated by reference in their entireties. In one aspect the polymeric carrier cargo complexes may comprise a negatively charged nucleic acid molecule such as, but not limited to, those described in International Patent Publication Nos. WO2013113325 and WO2013113502, the contents of each of which are herein incorporated by reference in its entirety.

As a non-limiting example, the core-shell nanoparticle may be used to treat an eye disease or disorder (See e.g. US Publication No. 20120321719, the contents of which are herein incorporated by reference in its entirety).

In one embodiment, the polymer used with the formulations described herein may be a modified polymer (such as, but not limited to, a modified polyacetal) as described in International Publication No. WO2011120053, the contents of which are herein incorporated by reference in its entirety.

### Peptides and Proteins

The antibody compositions of the invention can be formulated with peptides and/or proteins in order to increase transfection of cells by the polynucleotide. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention includes a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton Fla., 2002); El-Andaloussi et al., Curr. Pharm. Des. 11(28):3597-611 (2003); and Deshayes et al., Cell. Mol. Life Sci. 62(16):1839-49 (2005), all of which are incorporated herein by reference in their entirety). The compositions can also be formulated to include a cell penetrating agent, e.g., liposomes, which enhance delivery of the compositions to the intracellular space. Antibody compositions of the invention may be complexed to peptides and/or proteins such as, but not limited to, peptides and/or proteins from Aileron Therapeutics (Cambridge, Mass.) and Permeon Biologics (Cambridge, Mass.) in order to enable intracellular delivery (Cronican et al., ACS Chem. Biol. 2010 5:747-752; McNaughton et al., Proc. Natl. Acad. Sci. USA 2009 106:6111-6116; Sawyer, Chem Biol Drug Des. 2009 73:3-6; Verdine and Hilinski, Methods Enzymol. 2012; 503:3-33; all of which are herein incorporated by reference in its entirety).

In one embodiment, the cell-penetrating polypeptide may comprise a first domain and a second domain. The first domain may comprise a supercharged polypeptide. The second domain may comprise a protein-binding partner. As used herein, “protein-binding partner” includes, but are not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where the polynucleotide may be introduced.

Formulations of the including peptides or proteins may be used to increase cell transfection by the antibody composition, alter the biodistribution of the polynucleotide (e.g., by targeting specific tissues or cell types), and/or increase the translation of encoded protein. (See e.g., International Pub. No. WO2012110636 and WO2013123298; the contents of which are herein incorporated by reference in its entirety).

In one embodiment, the cell penetrating peptide may be, but is not limited to, those described in US Patent Publication No US20130129726, US20130137644 and US20130164219, each of which is herein incorporated by reference in its entirety.

### Cells

The antibody compositions of the invention can be transfected ex vivo into cells, which are subsequently transplanted into a subject. As non-limiting examples, the pharmaceutical compositions may include red blood cells to deliver modified RNA to liver and myeloid cells, virosomes to deliver modified RNA in virus-like particles (VLPs), and electroporated cells such as, but not limited to, from MAXCYTE® (Gaithersburg, Md.) and from ERYTECH® (Lyon, France) to deliver modified RNA. Examples of use of red blood cells, viral particles and electroporated cells to deliver payloads other than polynucleotides have been documented (Godfrin et al., Expert Opin Biol Ther. 2012 12:127-133; Fang et al., Expert Opin Biol Ther. 2012 12:385-389; Hu et al., Proc Natl Acad Sci USA. 2011 108:10980-10985; Lund et al., Pharm Res. 2010 27:400-420; Huckriede et al., J Liposome Res. 2007; 17:39-47; Cusi, Hum Vaccin. 2006 2:1-7; de Jonge et al., Gene Ther. 2006 13:400-411; all of which are herein incorporated by reference in its entirety).

The antibody compositions may be delivered in synthetic VLPs synthesized by the methods described in International Pub No. WO2011085231 and WO2013116656 and US Pub No. 20110171248, the contents of each of which are herein incorporated by reference in their entireties.

Cell-based formulations of the antibody compositions of the invention may be used to ensure cell transfection (e.g., in the cellular carrier), alter the biodistribution of the polynucleotide (e.g., by targeting the cell carrier to specific tissues or cell types), and/or increase the translation of encoded protein.

Introduction into Cells

A variety of methods are known in the art and suitable for introduction of nucleic acid into a cell, including viral and non-viral mediated techniques and any of these may be used to introduce the antibody compositions of the present invention. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

The technique of sonoporation, or cellular sonication, is the use of sound (e.g., ultrasonic frequencies) for modifying the permeability of the cell plasma membrane. Sonoporation methods are known to those in the art and are used to deliver nucleic acids in vivo (Yoon and Park, Expert Opin Drug Deliv. 2010 7:321-330; Postema and Gilja, Curr Pharm Biotechnol. 2007 8:355-361; Newman and Bettinger, Gene Ther. 2007 14:465-475; all herein incorporated by reference in their entirety). Sonoporation methods are known in the art and are also taught for example as it relates to bacteria in US Patent Publication 20100196983 and as it relates to other cell types in, for example, US Patent Publication 20100009424, each of which are incorporated herein by reference in their entirety.

Electroporation techniques are also well known in the art and are used to deliver nucleic acids in vivo and clinically (Andre et al., Curr Gene Ther. 2010 10:267-280; Chiarella et al., Curr Gene Ther. 2010 10:281-286; Hojman, Curr Gene Ther. 2010 10:128-138; all herein incorporated by reference in their entirety). Electroporation devices are sold by many companies worldwide including, but not limited to BTX® Instruments (Holliston, Mass.) (e.g., the AgilePulse In Vivo System) and Inovio (Blue Bell, Pa.) (e.g., Inovio SP-5P intramuscular delivery device or the CELLECTRA® 3000 intradermal delivery device). In one embodiment, antibody compositions may be delivered by using electroporation.

### Micro-Organ

The antibody compositions may be contained in a micro-organ which can then express an encoded polypeptide of interest in a long-lasting therapeutic formulation. In one aspect, the micro-organ may comprise a vector comprising a nucleic acid sequence (e.g., a polynucleotides of the present invention) encoding a polypeptide of interest, operably linked to one or more regulatory sequences. As a non-limiting example, the long-lasting therapeutic micro-organ used with the present invention may be those described in U.S. Pat. No. 845948, the contents of which are herein incorporated by reference in its entirety. As another non-limiting example, the micro-organ may be used to maintain a desired level of a polypeptide of interest for a sustained period of time (e.g., maintaining physiological hemoglobin levels as described in U.S. Pat. No. 845948, the contents of which are herein incorporated by reference in its entirety).

The micro-organ may be able to produce the polypeptide of interest for at least a day, at least two days, at least three days, at least four days, at least five days, at least six days, a least 7 days, at least 8 days, at least 9 days, at least 10 days, at least 11 days, at least 12 days, at least 13 days, at least 14 days, at least 3 weeks, at least 1 month and/or at least 2 months, at least 3 months, at least 4 months, at least 5 months, at least 6 months or greater than 6 months.

In one embodiment, the micro-organ may have a diameter of at least 0.5 mm to at least 20 mm such as, but not limited to, at least 0.5 mm, at least 1 mm, at least 1.5 mm, at least 2 mm, at least 2.5 mm, at least 3 mm, at least 3.5 mm, at least 4 mm, at least 4.5 mm, at least 5 mm, at least 5.5 mm, at least 6 mm, at least 6.5 mm, at least 7 mm, at least 7.5 mm, at least 8 mm, at least 8.5 mm, at least 9 mm, at least 9.5 mm, at least 10 mm, at least 10.5 mm, at least 11 mm, at least 11.5 mm, at least 12 mm, at least 12.5 mm, at least 13 mm, at least 13.5 mm, at least 14 mm, at least 14.5 mm, at least 15 mm, at least 15.5. mm, at least 16 mm, at least 16.5 mm, at least 17 mm, at least 17.5 mm, at least 18 mm, at least 18.5 mm, at least 19 mm, at least 19.5 mm or at least 20 mm. In another embodiment, the micro-organ may have a diameter of 0.5-2.5 mm, 1-2.5 mm, 1.5-2.5 mm, 0.5-3 mm, 1-3 mm, 1.5-3 mm, 0.5-3.5 mm, 1-3.5 mm, 1.5-3.5 mm, 0.5-4 mm, 1-4 mm, 1.5-4 mm, 2-4 mm, 0.5-5 mm, 1-5 mm, 1.5-5 mm, 2-5 mm, 2.5-5 mm, 3-5 mm, 0.5-6 mm, 1-6 mm, 1.5-6 mm, 2-6 mm, 2.5-6 mm, 3-6 mm, 3.5-6 mm, 4-6 mm, 0.5-7 mm, 1-7 mm, 1.5-7 mm, 2-7 mm, 2.5-7 mm, 3-7 mm, 3.5-7 mm, 4-7 mm, 4.5-7 mm, 5-7 mm, 0.5-8 mm, 1-8 mm, 1.5-8 mm, 2-8 mm, 2.5-8 mm, 3-8 mm, 3.5-8 mm, 4-8 mm, 4.5-8 mm, 5-8 mm, 5.5-8 mm, 6-8 mm, 0.5-9 mm, 1-9 mm, 1.5-9 mm, 2-9 mm, 2.5-9 mm, 3-9 mm, 3.5-9 mm, 4-9 mm, 4.5-9 mm, 5-9 mm, 5.5-9 mm, 6-9 mm, 6.5-9 mm, 7-9 mm, 0.5-10 mm, 1-10 mm, 1.5-10 mm, 2-10 mm, 2.5-10 mm, 3-10 mm, 3.5-10 mm, 4-10 mm, 4.5-10 mm, 5-10 mm, 5.5-10 mm, 6-10 mm, 6.5-10 mm, 7-10 mm, 7.5-10 nm or 8-10 nm.

In one embodiment, the micro-organ may have a length of at least 2 mm to at least 150 mm such as, but not limited to, at least 2 mm, at least 3 mm, at least 4 mm, at least 5 mm, at least 6 mm, at least 7 mm, at least 8 mm, at least 9 mm, at least 10 mm, at least 15 mm, at least 20 mm, at least 25 mm, at least 30 mm, at least 35 mm, at least 40 mm, at least 45 mm, at least 50 mm, at least 55 mm, at least 60 mm, at least 65 mm, at least 70 mm, at least 75 mm, at least 80 mm, at least 85 mm, at least 90 mm, at least 95 mm, at least 100 mm, at least 105 mm, at least 110 mm, at least 115 mm, at least 120 mm, at least 125 mm, at least 130 mm, at least 135 mm, at least 140 mm, at least 145 mm or at least 150 mm. In another embodiment, the micro-organ may have a length of 5-100 mm, 10-100 mm, 15-100 mm, 20-100 mm, 25-10 mm, 30-100 mm, 35-100 mm, 40-100 mm, 45-100 mm, 50-100 mm, 55-100 mm, 60-100 mm, 65-100 mm, 70-100 mm, 75-100 mm, 80-100 mm, 85-100 mm, 90-100 mm, 5-90 mm, 10-90 mm, 15-90 mm, 20-90 mm, 25-10 mm, 30-90 mm, 35-90 mm, 40-90 mm, 45-90 mm, 50-90 mm, 55-90 mm, 60-90 mm, 65-90 mm, 70-90 mm, 75-90 mm, 80-90 mm, 5-80 mm, 10-80 mm, 15-80 mm, 20-80 mm, 25-10 mm, 30-80 mm, 35-80 mm, 40-80 mm, 45-80 mm, 50-80 mm, 55-80 mm, 60-80 mm, 65-80 mm, 70-80 mm, 5-70 mm, 10-70 mm, 15-70 mm, 20-70 mm, 25-10 mm, 30-70 mm, 35-70 mm, 40-70 mm, 45-70 mm, 50-70 mm, 55-70 mm, 60-70 mm, 5-60 mm, 10-60 mm, 15-60 mm, 20-60 mm, 25-10 mm, 30-60 mm, 35-60 mm, 40-60 mm, 45-60 mm, 50-60 mm, 5-50 mm, 10-50 mm, 15-50 mm, 20-50 mm, 25-10 mm, 30-50 mm, 35-50 mm, 40-50 mm, 5-40 mm, 10-40 mm, 15-40 mm, 20-40 mm, 25-10 mm, 30-40 mm, 5-30 mm, 10-30 mm, 15-30 mm, 20-30 mm, 5-20 mm, 10-20 mm or 5-10 mm.

### Hyaluronidase

The intramuscular or subcutaneous localized injection of antibody compositions of the invention can include hyaluronidase, which catalyzes the hydrolysis of hyaluronan. By catalyzing the hydrolysis of hyaluronan, a constituent of the interstitial barrier, hyaluronidase lowers the viscosity of hyaluronan, thereby increasing tissue permeability (Frost, Expert Opin. Drug Deliv. (2007) 4:427-440; herein incorporated by reference in its entirety). It is useful to speed their dispersion and systemic distribution of encoded proteins produced by transfected cells. Alternatively, the hyaluronidase can be used to increase the number of cells exposed to a polynucleotide of the invention administered intramuscularly or subcutaneously.

### Nanoparticle Mimics

The antibody compositions of the invention may be encapsulated within and/or absorbed to a nanoparticle mimic. A nanoparticle mimic can mimic the delivery function organisms or particles such as, but not limited to, pathogens, viruses, bacteria, fungus, parasites, prions and cells. As a non-limiting example the antibody compositions of the invention may be encapsulated in a non-viron particle which can mimic the delivery function of a virus (see International Pub. No. WO2012006376 and US Patent Publication No. US20130171241 and US20130195968, the contents of each of which are herein incorporated by reference in its entirety).

### Nanotubes

The antibody compositions of the invention can be attached or otherwise bound to at least one nanotube such as, but not limited to, rosette nanotubes, rosette nanotubes having twin bases with a linker, carbon nanotubes and/or single-walled carbon nanotubes, The antibody compositions may be bound to the nanotubes through forces such as, but not limited to, steric, ionic, covalent and/or other forces.

In one embodiment, the nanotube can release one or more antibody compositions into cells. The size and/or the surface structure of at least one nanotube may be altered so as to govern the interaction of the nanotubes within the body and/or to attach or bind to the antibody compositions disclosed herein. In one embodiment, the building block and/or the functional groups attached to the building block of the at least one nanotube may be altered to adjust the dimensions and/or properties of the nanotube. As a non-limiting example, the length of the nanotubes may be altered to hinder the nanotubes from passing through the holes in the walls of normal blood vessels but still small enough to pass through the larger holes in the blood vessels of tumor tissue.

In one embodiment, at least one nanotube may also be coated with delivery enhancing compounds including polymers, such as, but not limited to, polyethylene glycol. In another embodiment, at least one nanotube and/or the antibody compositions may be mixed with pharmaceutically acceptable excipients and/or delivery vehicles.

In one embodiment, the antibody compositions are attached and/or otherwise bound to at least one rosette nanotube. The rosette nanotubes may be formed by a process known in the art and/or by the process described in International Publication No. WO2012094304, herein incorporated by reference in its entirety. At least one antibody composition may be attached and/or otherwise bound to at least one rosette nanotube by a process as described in International Publication No. WO2012094304, herein incorporated by reference in its entirety, where rosette nanotubes or modules forming rosette nanotubes are mixed in aqueous media with at least one antibody composition under conditions which may cause at least one antibody compositions to attach or otherwise bind to the rosette nanotubes.

In one embodiment, the antibody compositions may be attached to and/or otherwise bound to at least one carbon nanotube. As a non-limiting example, the antibody compositions may be bound to a linking agent and the linked agent may be bound to the carbon nanotube (See e.g., U.S. Pat. No. 8,246,995; herein incorporated by reference in its entirety). The carbon nanotube may be a single-walled nanotube (See e.g., U.S. Pat. No. 8,246,995; herein incorporated by reference in its entirety).

### Conjugates

The antibody compositions of the invention include conjugates, such as a polynucleotide covalently linked to a carrier or targeting group, or including two encoding regions that together produce a fusion protein (e.g., bearing a targeting group and therapeutic protein or peptide).

The conjugates of the invention include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymers, or polyphosphazene. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Representative U.S. patents that teach the preparation of polynucleotide conjugates, particularly to RNA, include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; each of which is herein incorporated by reference in their entireties.

In one embodiment, the conjugate of the present invention may function as a carrier for the antibody compositions of the present invention. The conjugate may comprise a cationic polymer such as, but not limited to, polyamine, polylysine, polyalkylenimine, and polyethylenimine which may be grafted to with poly(ethylene glycol). As a non-limiting example, the conjugate may be similar to the polymeric conjugate and the method of synthesizing the polymeric conjugate described in U.S. Pat. No. 6,586,524 herein incorporated by reference in its entirety.

A non-limiting example of a method for conjugation to a substrate is described in US Patent Publication No. US20130211249, the contents of which are herein incorporated by reference in its entirety. The method may be used to make a conjugated polymeric particle comprising an antibody composition.

The conjugates can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Targeting groups may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The ligand can be, for example, a lipopolysaccharide, or an activator of p38 MAP kinase.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein.

As a non-limiting example, the targeting group may be a glutathione receptor (GR)-binding conjugate for targeted delivery across the blood-central nervous system barrier (See e.g., US Patent Publication No. US2013021661012, the contents of which are herein incorporated by reference in its entirety.

In one embodiment, the conjugate of the present invention may be a synergistic biomolecule-polymer conjugate. The synergistic biomolecule-polymer conjugate may be long-acting continuous-release system to provide a greater therapeutic efficacy. The synergistic biomolecule-polymer conjugate may be those described in US Patent Publication No. US20130195799, the contents of which are herein incorporated by reference in its entirety.

In another embodiment, the conjugate which may be used in the present invention may be an aptamer conjugate. Non-limiting examples of aptamer conjugates are described in International Patent Publication No. WO2012040524, the contents of which are herein incorporated by reference in its entirety. The aptamer conjugates may be used to provide targeted delivery of formulations comprising antibody compositions.

In one embodiment, the conjugate which may be used in the present invention may be an amine containing polymer conjugate. Non-limiting examples of amine containing polymer conjugate are described in U.S. Pat. No. 8,507,653, the contents of which are herein incorporated by reference in its entirety. The factor IX moiety polymer conjugate may be ucomprise releasable linkages to release the antibody compositions upon and/or after delivery to a subject.

In one embodiment, pharmaceutical compositions of the present invention may include chemical modifications such as, but not limited to, modifications similar to locked nucleic acids.

Representative U.S. Patents that teach the preparation of locked nucleic acid (LNA) such as those from Santaris, include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include polynucleotides with phosphorothioate backbones and oligonucleosides with other modified backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P(O)₂—O—CH₂—] of the above-referenced U.S. Pat. No. 5,489,677, and the amide backbones of the above-referenced U.S. Pat. No. 5,602,240. In some embodiments, the polynucletotides featured herein have morpholino backbone structures of the above-referenced U.S. Pat. No. 5,034,506.

Modifications at the 2′ position may also aid in delivery. Preferably, modifications at the 2′ position are not located in a polypeptide-coding sequence, i.e., not in a translatable region. Modifications at the 2′ position may be located in a 5′UTR, a 3′UTR and/or a tailing region. Modifications at the 2′ position can include one of the following at the 2′ position: H (i.e., 2′-deoxy); F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH2).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, the polynucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties, or a group for improving the pharmacodynamic properties, and other substituents having similar properties. In some embodiments, the modification includes a 2′-methoxyethoxy (2′-O-CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below. Other modifications include 2′-methoxy (2′-OCH₃), 2′-aminopropoxy (2′-OCH₂CH₂CH₂NH₂) and 2′-fluoro (2′-F). Similar modifications may also be made at other positions, particularly the 3′ position of the sugar on the 3′ terminal nucleotide or in 2′-5′ linked dsRNAs and the 5′ position of 5′ terminal nucleotide. Polynucleotides of the invention may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920; the contents of each of which is herein incorporated by reference in their entirety.

In one embodiment, the antibody compositions may be conjugated to an agent to enhance delivery. As a non-limiting example, the agent may be a monomer or polymer such as a targeting monomer or a polymer having targeting blocks as described in International Publication No. WO2011062965, herein incorporated by reference in its entirety. In another non-limiting example, the agent may be a transport agent covalently coupled to the polynucleotides of the present invention (See e.g., U.S. Pat. Nos. 6,835,393 and 7,374,778, each of which is herein incorporated by reference in its entirety). In yet another non-limiting example, the agent may be a membrane barrier transport enhancing agent such as those described in U.S. Pat. Nos. 7,737,108 and 8,003,129, each of which is herein incorporated by reference in its entirety.

In another embodiment, polynucleotides may be conjugated to SMARTT POLYMER TECHNOLOGY® (PHASERX®, Inc. Seattle, Wash.).

In another aspect, the conjugate may be a peptide that selectively directs the nanoparticle to neurons in a tissue or organism. As a non-limiting example, the peptide used may be, but is not limited to, the peptides described in US Patent Publication No US20130129627, herein incorporated by reference in its entirety.

In yet another aspect, the conjugate may be a peptide that can assist in crossing the blood-brain barrier.

### Self-Assembled Nanoparticles

### Nucleic Acid Self-Assembled Nanoparticles

Self-assembled nanoparticles have a well-defined size which may be precisely controlled as the nucleic acid strands may be easily reprogrammable. For example, the optimal particle size for a cancer-targeting nanodelivery carrier is 20-100 nm as a diameter greater than 20 nm avoids renal clearance and enhances delivery to certain tumors through enhanced permeability and retention effect. Using self-assembled nucleic acid nanoparticles a single uniform population in size and shape having a precisely controlled spatial orientation and density of cancer-targeting ligands for enhanced delivery. As a non-limiting example, oligonucleotide nanoparticles were prepared using programmable self-assembly of short DNA fragments and therapeutic siRNAs. These nanoparticles are molecularly identical with controllable particle size and target ligand location and density. The DNA fragments and siRNAs self-assembled into a one-step reaction to generate DNA/siRNA tetrahedral nanoparticles for targeted in vivo delivery. (Lee et al., Nature Nanotechnology 2012 7:389-393; herein incorporated by reference in its entirety).

In one embodiment, the antibody compositions disclosed herein may be formulated as self-assembled nanoparticles. As a non-limiting example, nucleic acids may be used to make nanoparticles which may be used in a delivery system for the antibody compositions of the present invention (See e.g., International Pub. No. WO2012125987; herein incorporated by reference in its entirety).

In one embodiment, the nucleic acid self-assembled nanoparticles may comprise a core of the antibody compositions disclosed herein and a polymer shell. The polymer shell may be any of the polymers described herein and are known in the art. In an additional embodiment, the polymer shell may be used to protect the antibody compositions in the core.

The metallic nanoparticle which may be used in the present invention may be a pH-sensitive nanoparticle such as, but not limited to, those described in US Patent Publication No US20130138032, herein incorporated by reference in its entirety.

In one aspect, the metallic and/or metal-allow nanoparticles may be made by the methods described in US Patent Publication No US20130133483, herein incorporated by reference in its entirety

### Polymer-Based Self-Assembled Nanoparticles

Polymers may be used to form sheets which self-assembled into nanoparticles. These nanoparticles may be used to deliver the antibody compositions of the present invention. In one embodiment, these self-assembled nanoparticles may be microsponges formed of long polymers of RNA hairpins which form into crystalline ‘pleated’ sheets before self-assembling into microsponges. These microsponges are densely-packed sponge like microparticles which may function as an efficient carrier and may be able to deliver cargo to a cell. The microsponges may be from 1 um to 300 nm in diameter. The microsponges may be complexed with other agents known in the art to form larger microsponges. As a non-limiting example, the microsponge may be complexed with an agent to form an outer layer to promote cellular uptake such as polycation polyethyleneime (PEI). This complex can form a 250-nm diameter particle that can remain stable at high temperatures (150° C.) (Grabow and Jaegar, Nature Materials 2012, 11:269-269; herein incorporated by reference in its entirety). Additionally these microsponges may be able to exhibit an extraordinary degree of protection from degradation by ribonucleases.

In another embodiment, the polymer-based self-assembled nanoparticles such as, but not limited to, microsponges, may be fully programmable nanoparticles. The geometry, size and stoichiometry of the nanoparticle may be precisely controlled to create the optimal nanoparticle for delivery of cargo such as, but not limited to, antibody compositions.

In yet another embodiment, the polymer based nanoparticle may comprise a non-nucleic acid polymer comprising a plurality of heterogenous monomers such as those described in International Publication No. WO2013009736, the contents of which are herein incorporated by reference in its entirety.

### Self-Assembled Macromolecules

The antibody compositions may be formulated in amphiphilic macromolecules (AMs) for delivery. AMs comprise biocompatible amphiphilic polymers which have an alkylated sugar backbone covalently linked to poly(ethylene glycol). In aqueous solution, the AMs self-assemble to form micelles. Non-limiting examples of methods of forming AMs and AMs are described in US Patent Publication No. US20130217753, the contents of which are herein incorporated by reference in its entirety.

### Inorganic Nanoparticles

The antibody compositions of the present invention may be formulated in inorganic nanoparticles (U.S. Pat. No. 8,257,745, herein incorporated by reference in its entirety). The inorganic nanoparticles may include, but are not limited to, clay substances that are water swellable. As a non-limiting example, the inorganic nanoparticle may include synthetic smectite clays which are made from simple silicates (See e.g., U.S. Pat. Nos. 5,585,108 and 8,257,745 each of which are herein incorporated by reference in their entirety).

In one embodiment, the inorganic nanoparticles may comprise a core of the antibody compositions disclosed herein and a polymer shell. The polymer shell may be any of the polymers described herein and are known in the art. In an additional embodiment, the polymer shell may be used to protect the antibody compositions in the core.

### Semi-Conductive and Metallic Nanoparticles

The antibody compositions of the present invention may be formulated in water-dispersible nanoparticle comprising a semiconductive or metallic material (U.S. Pub. No. 20120228565; herein incorporated by reference in its entirety) or formed in a magnetic nanoparticle (U.S. Pub. No. 20120265001 and 20120283503; each of which is herein incorporated by reference in its entirety). The water-dispersible nanoparticles may be hydrophobic nanoparticles or hydrophilic nanoparticles.

In one embodiment, the semi-conductive and/or metallic nanoparticles may comprise a core of the antibody compositions disclosed herein and a polymer shell. The polymer shell may be any of the polymers described herein and are known in the art. In an additional embodiment, the polymer shell may be used to protect the antibody compositions in the core.

### Surgical Sealants: Gels and Hydrogels

In one embodiment, the antibody compositions disclosed herein may be encapsulated into any hydrogel known in the art which may form a gel when injected into a subject. Hydrogels are a network of polymer chains that are hydrophilic, and are sometimes found as a colloidal gel in which water is the dispersion medium. Hydrogels are highly absorbent (they can contain over 99% water) natural or synthetic polymers. Hydrogels also possess a degree of flexibility very similar to natural tissue, due to their significant water content. The hydrogel described herein may used to encapsulate lipid nanoparticles which are biocompatible, biodegradable and/or porous. A hydrogel can be made in situ from solution injection or implanted.

As a non-limiting example, the hydrogel may be an aptamer-functionalized hydrogel. The aptamer-functionalized hydrogel may be programmed to release one or more polynucleotides using nucleic acid hybridization. (Battig et al., J. Am. Chem. Society. 2012 134:12410-12413; the contents of which is herein incorporated by reference in its entirety).

As another non-limiting example, the hydrogel may be a shaped as an inverted opal. The opal hydrogels exhibit higher swelling ratios and the swelling kinetics is an order of magnitude faster than conventional hydrogels as well. Methods of producing opal hydrogels and description of opal hydrogels are described in International Pub. No. WO2012148684, the contents of which is herein incorporated by reference in its entirety.

In yet another non-limiting example, the hydrogel may be an antibacterial hydrogel. The antibacterial hydrogel may comprise a pharmaceutical acceptable salt or organic material such as, but not limited to pharmaceutical grade and/or medical grade silver salt and aloe vera gel or extract. (International Pub. No. WO2012151438, the contents of which are herein incorporated by reference in its entirety).

In one embodiment, an antibody composition may be encapsulated in a lipid nanoparticle and then the lipid nanoparticle may be encapsulated into a hydrogel.

In one embodiment, the antibody compositions disclosed herein may be encapsulated into any gel known in the art. As a non-limiting example the gel may be a fluorouracil injectable gel or a fluorouracil injectable gel containing a chemical compound and/or drug known in the art. As another example, the antibody compositions may be encapsulated in a fluorouracil gel containing epinephrine (See e.g., Smith et al. Cancer Chemotherapty and Pharmacology, 1999 44(4):267-274; the contents of which are herein incorporated by reference in its entirety).

In one embodiment, the antibody compositions disclosed herein may be encapsulated into a fibrin gel, fibrin hydrogel or fibrin glue.

In another embodiment, the antibody compositions may be formulated in a lipid nanoparticle or a rapidly eliminated lipid nanoparticle prior to being encapsulated into a fibrin gel, fibrin hydrogel or a fibrin glue. In yet another embodiment, the antibody compositions may be formulated as a lipoplex prior to being encapsulated into a fibrin gel, hydrogel or a fibrin glue. Fibrin gels, hydrogels and glues comprise two components, a fibrinogen solution and a thrombin solution which is rich in calcium (See e.g., Spicer and Mikos, Journal of Controlled Release 2010. 148: 49-55; Kidd et al. Journal of Controlled Release 2012. 157:80-85; each of which is herein incorporated by reference in its entirety). The concentration of the components of the fibrin gel, hydrogel and/or glue can be altered to change the characteristics, the network mesh size, and/or the degradation characteristics of the gel, hydrogel and/or glue such as, but not limited to changing the release characteristics of the fibrin gel, hydrogel and/or glue. (See e.g., Spicer and Mikos, Journal of Controlled Release 2010. 148: 49-55; Kidd et al. Journal of Controlled Release 2012. 157:80-85; Catelas et al. Tissue Engineering 2008. 14:119-128; each of which is herein incorporated by reference in its entirety). This feature may be advantageous when used to deliver the modified mRNA disclosed herein. (See e.g., Kidd et al. Journal of Controlled Release 2012. 157:80-85; Catelas et al. Tissue Engineering 2008. 14:119-128; each of which is herein incorporated by reference in its entirety).

In one embodiment, the antibody compositions disclosed herein may be used with hydrogels such as, but not limited to, the hydrogels described in U.S. Patent Application No. 20130071450 or 20130211249, the contents of each of which is herein incorporated by reference in its entirety.

As a non-limiting example, the hydrogels which may be used in the present invention may be made by the methods described in International Patent Publication No. WO2013124620, the contents of which are herein incorporated by reference in its entirety.

In another embodiment, the antibody compositions disclosed herein may be formulated for transdermal delivery. The formulation may comprise at least one hydrogel described in U.S. Patent Application No. 20130071450, the contents of which are herein incorporated by reference in its entirety.

In one embodiment, the hydrogel which may be used in the present invention is described in U.S. Pat. No. 8,420,605, U.S. Pat. No. 8,415,325 and/or International Patent Publication No. WO2013091001 and WO2013124620, the contents of each of which are herein incorporated by reference in its entirety.

In one embodiment, the hydrogel which may be used in the present invention may be, but is not limited to, ATRIGEL® (QLT Inc. Vancouver, British Columbia), chitosan, aliginate, collagen or hyaluronic acid hydrogel.

In another embodiment, the hydrogel which may be used in the present invention is a crosslinked methacrylate. As a non-limiting example, the hydrogel of the present invention may be used in wound dressings.

The hydrogel which may be used in the present invention may also be complexed with agents and excipients described herein including, but not limited to PEI, PVA, poly-lysine, Poloxamer 124, Poloxamer 181, Poloxamer 182, Poloxamer 407, Poloxamer 237, Poloxamer 331 and Poloxamer 338. Complexing the hydrogel with agents and/or excipients may help improve mRNA stability and uptake in a cell, tissue and/or organism. As a non-limiting example, a hydrogel may be complexed with Poloxamer 188 to improve the stability and uptake of mRNA.

In one embodiment, the antibody compositions disclosed herein may be formulated in a surgical sealant. The surgical sealant may be, but is not limited to, fibrinogen polymer based sealants (Ethicon Inc. Cornelia, Ga.), TISSELL® (Baxter International, Inc Deerfield, Ill.) or PEG-based sealants such as, but not limited to, COSEAL® (Baxter International, Inc Deerfield, Ill.) and DURASEAL™ (trilysine amine/PEG-ester) (Covidien, Waltham, Mass.).

In one embodiment, antibody compositions may be formulated in COSEAL® or co-administered with or administered after a cell, tissue or organism is administered COSEAL®. COSEAL® comprises two synthetic polyethylene glycols (PEGs) (pentaerythritol PEG ester tetra-succinimidyl and pentaerythritol PEG ether tetra-thiol), a dilute hydrogen chloride solution, and a sodium phosphate/sodium carbonate solution. The PEGs are kept separate from the sodium phosphate/sodium carbonate solution in the dilute hydrogen chloride solution until administration. After administration a hydrogel is formed, which may adhere to tissue, and forms a stiff gel in seconds which is resorbed within 30 days.

In another embodiment, the antibody compositions disclosed herein may be formulated in a hydrogel comprising a macromolecular matrix. The macromolecular matrix may comprise a hyaluronic acid component which may be crosslinked to a collagent component. The hydrogel used in the present invention may be, but is not limited to, the hydrogels described in International Patent Publication No. WO2013106715, the contents of which are herein incorporated by reference in its entirety.

In yet another embodiment, the antibody compositions disclosed herein may be formulated in a chitosan glycerophosphate (CGP) hydrogel. The formulation may further comprise a chitosanase in an effect amount to dissolve the CGP hydrogel and release the antibody compositions associated with the CGP hydrogel. As a non-limiting example, the antibody compositions may be formulated in the controlled release delivery system comprising a CGP hydrogel described in US Patent Publication No. US20130189241, the contents of which are herein incorporated by reference in its entirety.

In one embodiment, the antibody compositions disclosed herein may be formulated in a hydrogel formulated for controlled release such as, but not limited to, the porous matrix composites and formulations described in US Patent Publication No. US20130196915, the contents of which are herein incorporated by reference in its entirety.

In another embodiment, the antibody compositions disclosed herein may be formulated in a hydrogel comprising heterobifunctional poly(alkylene oxides) which may have degradable linkages. Non-limiting examples of heterobifunctional poly(alkylene oxides) are described in U.S. Pat. No. 8,497,357, the contents of which are herein incorporated by reference in its entirety.

In yet another embodiment, the antibody compositions may be formulated in a hydrogel which may be used as an insulin delivery system. As a non-limiting example, the hydrogel may be a glucose binding amphiphilic peptide hydrogel as described in International Patent Publication No. WO2013123491, the contents of which are herein incorporated by reference in its entirety. As another non-limiting example, the hydrogel may be a microgel such as the glucose-responsive microgels described in International Patent Publication No. WO2013123492, the contents of which are herein incorporated by reference in its entirety.

In one embodiment, the antibody compositions may be formulated in a hydrogel system such as, but not limited to, a multi-compartment hydrogel. A non-limiting example of a multi-compartment hydrogel and methods of making the hydrogel is described in International Patent Publication No. WO2013124855, the contents of which are herein incorporated by reference in its entirety. The multi-compartment hydrogel may be used to repair or regenerate damaged tissue in a subject.

In another embodiment, the antibody compositions may be formulated in a cucurbituril-based hydrogel. A non-limiting example of a cucurbituril-based hydrogel is described in international Patent Publication No. WO2013124654, the contents of which are herein incorporated by reference in its entirety.

In one embodiment, the antibody compositions disclosed herein may be formulated in a PEG-based surgical sealant or hydrogel.

In one embodiment, the surgical sealant or hydrogel may include at least one, at least two, at least three, at least four, at least five, at least six or more than six PEG lipids. The PEG lipids may be selected from, but are not limited to, pentaerythritol PEG ester tetra-succinimidyl and pentaerythritol PEG ether tetra-thiol, PEG-c-DOMG, PEG-DMG (1,2-Dimyristoyl-sn-glycerol, methoxypolyethylene Glycol), PEG-DSG (1,2-Distearoyl-sn-glycerol, methoxypolyethylene Glycol), PEG-DPG (1,2-Dipalmitoyl-sn-glycerol, methoxypolyethylene glycol), PEG-DSA (PEG coupled to 1,2-distearyloxypropyl-3-amine), PEG-DMA (PEG coupled to 1,2-dimyristyloxypropyl-3-amine, PEG-c-DNA, PEG-c-DMA, PEG-S-DSG, PEG-c-DMA, PEG-DPG, PEG-DMG 2000 and those described herein and/or known in the art. The concentration and/or ratio of the PEG lipids in the surgical sealant or hydrogel may be varied in order to optimize the formulation for delivery and/or administration.

The amount of buffer and/or acid used in combination with the PEG lipids of the surgical sealant or hydrogel may also be varied. In one non-limiting example, the ratio of buffer and/or acid with PEG lipids is 1:1. As a non-limiting example, the amount of buffer and/or acid used with the PEG lipids may be increased to alter the ratio of buffer/acid to PEG in order to optimize the surgical sealant or hydrogel. As another non-limiting example, the amount of buffer and/or acid used with the PEG lipids may be decreased to alter the ratio of buffer/acid to PEG in order to optimize the surgical sealant or hydrogel.

The amount of antibody compositions loaded into the buffer, acid and/or PEG lipid may be varied. The amount of antibody compositions loaded into the buffer, acid and/or PEG lipid may be, but is not limited to, at least 1 uL, at least 2 uL, at least 5 uL, at least 10 uL, at least 15 uL, at least 20 uL, at least 25 uL, at least 30 uL, at least 35 uL, at least 40 uL, at least 45 ul, at least 50 uL, at least 55 uL, at least 60 uL, at least 65 uL, at least 70 uL, at least 75 uL, at least 80 uL, at least 85 uL, at least 90 uL, at least 100 uL, at least 125 uL, at least 150 uL, at least 200 uL, at least 250 uL, at least 300 uL, at least 350 uL, at least 400 uL, at least 450 uL, at least 500 uL or more than 500 uL.

In one embodiment, the antibody compositions of the present invention may be loaded in PEGs and also in the buffer or the acid. The amount of antibody compositions loaded in the PEG may be the same, greater or less than the amount loaded in the buffer or acid. In another embodiment, the antibody compositions may be formulated, by the methods described herein and/or known in the art, prior to loading in the PEGs, buffer or acid.

A non-limiting example of a PEG-based hydrogel which may be used in the present invention is described in U.S. Pat. No. 8,524,215, the contents of which is herein incorporated by reference in its entirety. The PEG-based hydrogel may be an absorbable hydrogel prepared from a multi-arm PEG-vinyl sulfone having about 3 to about 8 arms and a multi-arm-PEG-R-sulfhydryl having about 3 to about 8 arms (See e.g., U.S. Pat. No. 8,524,215). In one embodiment, the PEG-based hydrogel may be an absorbable hydrogel. While not wishing to be bound by theory, an absorbable PEG-based hydrogel may be beneficial to reduce the permanent chronic foreign body reaction since the absorbable hydrogel can be absorbed and passed by the body.

In one embodiment, the hydrogel may be a thermosensitive hydrogel. In one aspect the thermosensitive hydrogel may be, but is not limited to, a triblock polymer such as those described herein and known in the art. As a non-limiting example, the tri-block polymer may be PEG-PLGA-PEG (see e.g., the thermosensitive hydrogel (PEG-PLGA-PEG) was used as a TGF-beta1 gene delivery vehicle in Lee et al. Thermosensitive Hydrogel as a Tgf-β1 Gene Delivery Vehicle Enhances Diabetic Wound Healing. Pharmaceutical Research, 2003 20(12): 1995-2000; as a controlled gene delivery system in Li et al. Controlled Gene Delivery System Based on Thermosensitive Biodegradable Hydrogel. Pharmaceutical Research 2003 20(6):884-888; and Chang et al., Non-ionic amphiphilic biodegradable PEG-PLGA-PEG copolymer enhances gene delivery efficiency in rat skeletal muscle. J Controlled Release. 2007 118:245-253; each of which is herein incorporated by reference in its entirety). As a non-limiting example, the thermosensitive hydrogel may be used to make nanoparticles and liposomes by the methods described in International Publication No. WO2013123407, the contents of which are herein incorporated by reference in its entirety.

In another embodiment, the hydrogel may be a biodegradable copolymer hydrogel (see e.g., the biodegradable hydrogels described by Nguyen and Lee (Injectable Biodegradable Hydrogels. Macromolecular Bioscience. 2010 10:563-579), herein incorporated by reference in its entirety). These hydrogels may exhibit a sol-gel phase transition that respond to external stimuli such as, but not limited to, temperature changes, pH alternations or both. Non-limiting examples of biodegradable copolymer hydrogels include triblock copolymers PEG-PLLA-PEG, PEG-PLA-PEG (see e.g., Chang et al., Non-ionic amphiphilic biodegradable PEG-PLGA-PEG copolymer enhances gene delivery efficiency in rat skeletal muscle. J Controlled Release. 2007 118:245-253, herein incorporated by reference in its entirety), PLGA-PEG-PLGA, PEG-PCL-PEG, PCL-PEG-PCL, polyesters such as poly[(R)-3-hydroxybutyrate] (PHB), polyphosphazenes such as L-sioleucine ethyl ester (IleOEt), D,L-leucine ethyl ester (LeuOEt), L-valine ethyl ester (ValOEt), or di-, tri- and oligo-peptides, polypeptides and chitosan. Temperature and pH sensitive polymers which may be used to form the biodegradable copolymer hydrogels include, but are not limited to, sulfamethazine-, poly(β-amino ester)-, poly(amino urethane)-, and poly(amidoamine)-based polymers. Formulations of the biodegradable copolymer hydrogels and antibody compositions may be administered using site-specific control of release behavior.

In one embodiment, the hydrogel used in the present invention may be a PEG based hydrogel such as, but not limited to, those described in International Patent Publication No WO2013082590, herein incorporated by reference in its entirety. The PEG based hydrogel may have, but is not limited to, an overall polymer weight concentration of less than or equal to 50% at the time of curing. As a non-limiting example, the PEG based hydrogel may be made by the methods described in International Patent Publication No WO2013082590, the contents of which are herein incorporated by reference in its entirety.

In another embodiment, the antibody compositions may be formulated in a nanostructured gel composition. The nanostructured gel may be capable of controlled release of the encapsulated antibody compositions. Non-limiting examples of nanostructed gels or self-assembled gels are described in International Patent Publication No. WO2012040623, the contents of which are herein incorporated by reference in its entirety.

In one embodiment, the concentration of the antibody compositions of the present invention in the surgical sealants, gels and/or hydrogels may be selected to provide a dosage within the range to have the desired therapeutic effect.

In one embodiment, the concentration of the polynucleotides of the antibody composition of the present invention in the surgical sealants, gels and/or hydrogels may be at least 0.001 mg to at least 150 mg in at least 0.1 ml to at least 30 ml of the surgical sealant, gel or hydrogel. The concentration of the polynucleotides of the present invention may be at least 0.001 mg, at least 0.005 mg, at least 0.01 mg, at least 0.05 mg, at least 0.1 mg, at least 0.5 mg, at least 1 mg, at least 5 mg, at least 7 mg, at least 10 mg, at least 12, at least 15 mg, at least 17 mg, at least 20 mg, at least 22 mg, at least 25 mg, at least 27 mg, at least 30 mg, at least 32 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg or at least 150 mg in at least 0.1 ml, at least 0.2 ml, at least 0.3 ml, at least 0.4 ml, at least 0.5 ml, at least 0.6 ml, at least 0.7 ml, at least 0.8 ml, at least 0.9 ml, at least 1 ml, at least 2 ml, at least 3 ml, at least 4 ml, at least 5 ml, at least 6 ml, at least 7 ml, at least 8 ml, at least 9 ml, at least 10 ml, at least 11 ml, at least 12 ml, at least 13 ml, at least 14 ml, at least 15 ml, at least 16 ml, at least 17 ml, at least 18 ml, at least 19 ml, at least 20 ml, at least 21 ml, at least 22 ml, at least 23 ml, at least 24 ml, at least 25 ml, at least 26 ml, at least 27 ml, at least 28 ml, at least 29 ml or at least 30 ml of the surgical sealant, gel or hydrogel.

In another embodiment, concentration of the polynucleotides of the antibody composition of the present invention in the surgical sealants, gels and/or hydrogels may be at least 0.001 mg/ml at least 0.005 mg/ml, at least 0.01 mg/ml, at least 0.05 mg/ml, at least 0.1 mg/ml, at least 0.5 mg/ml, at least 1 mg/ml, at least 5 mg/ml, at least 7 mg/ml, at least 10 mg/ml, at least 12, at least 15 mg/ml, at least 17 mg/ml, at least 20 mg/ml, at least 22 mg/ml, at least 25 mg/ml, at least 27 mg/ml, at least 30 mg/ml, at least 32 mg/ml, at least 35 mg/ml, at least 40 mg/ml, at least 45 mg/ml or at least 50 mg/ml.

Technology allowing for large subcutaneous infusion volumes which are known in the art, such as, but not limited to, HYLENEX® (Halozyme Therapeutics, San Diego, Calif.) may also be used. The dispersion and/or adsorption of the modified mRNA described herein may be increased with the use of HYLENEX® as HYLENEX® temporarily breaks down hyaluronic acid causing a temporary degradation in the subcutaneous space (for about 24 hours) just beneath the outside surface of the skin opening microscopic channels and allowing fluid or drugs to be dispersed and absorbed in the body.

In one embodiment, the hydrogel is a PEG based hydrogel which may be used for a topical application (See e.g., US Patent Publication No. US20130149318, herein incorporated by reference in its entirety).

In another embodiment, the hydrogel is an absorbable hydrogel. The absorbably hydrogel may be a PEG-based hydrogel as described in and/or made by the methods described in International Publication No. WO2012018718, the contents of which are herein incorporated by reference in its entirety. The absorbable hydrogels may be used to form sustained release compositions for use with the present invention (see e.g., International Pub. No. WO2012018718, the contents of which are herein incorporated by reference in its entirety).

In one embodiment, the hydrogel may comprise a polymer described in International Publication No. WO2013091001, the contents of which are herein incorporated by reference in its entirety.

### Suspension Formulations

In some embodiments, suspension formulations are provided comprising antibody compositions, water immiscible oil depots, surfactants and/or co-surfactants and/or co-solvents. Combinations of oils and surfactants may enable suspension formulation with antibody compositions. Delivery of antibody compositions in a water immiscible depot may be used to improve bioavailability through sustained release of mRNA from the depot to the surrounding physiologic environment and prevent polynucleotides degradation by nucleases.

In some embodiments, suspension formulations of antibody composition may be prepared using combinations of polynucleotides, oil-based solutions and surfactants. Such formulations may be prepared as a two-part system comprising an aqueous phase comprising polynucleotides and an oil-based phase comprising oil and surfactants. Exemplary oils for suspension formulations may include, but are not limited to sesame oil and Miglyol (comprising esters of saturated coconut and palmkernel oil-derived caprylic and capric fatty acids and glycerin or propylene glycol), corn oil, soybean oil, peanut oil, beeswax and/or palm seed oil. Exemplary surfactants may include, but are not limited to Cremophor, polysorbate 20, polysorbate 80, polyethylene glycol, transcutol, Capmul®, labrasol, isopropyl myristate, and/or Span 80. In some embodiments, suspensions may comprise co-solvents including, but not limited to ethanol, glycerol and/or propylene glycol.

Suspensions may be formed by first preparing an antibody composition formulation comprising an aqueous solution of polynucleotide and an oil-based phase comprising one or more surfactants. Suspension formation occurs as a result of mixing the two phases (aqueous and oil-based). In some embodiments, such a suspension may be delivered to an aqueous phase to form an oil-in-water emulsion. In some embodiments, delivery of a suspension to an aqueous phase results in the formation of an oil-in-water emulsion in which the oil-based phase comprising polynucleotides forms droplets that may range in size from nanometer-sized droplets to micrometer-sized droplets.

In some embodiments, specific combinations of oils, surfactants, cosurfactants and/or co-solvents may be utilized to suspend antibody compositions in the oil phase and/or to form oil-in-water emulsions upon delivery into an aqueous environment.

In some embodiments, suspensions may provide modulation of the release of antibody compositions into the surrounding environment. In such embodiments, antibody release may be modulated by diffusion from a water immiscible depot followed by resolubilization into a surrounding environment (e.g. an aqueous environment).

In some embodiments, antibody compositions within a water immiscible depot (e.g. suspended within an oil phase) may result in altered polynucleotides stability (e.g. altered degradation by nucleases).

In some embodiments, antibody compositions may be formulated such that upon injection, an emulsion forms spontaneously (e.g. when delivered to an aqueous phase). Such particle formation may provide a high surface area to volume ratio for release of polynucleotides from an oil phase to an aqueous phase.

In one embodiment, the antibody compositions may be formulated in a nanoemulsion such as, but not limited to, the nanoemulsions described in U.S. Pat. No. 8,496,945, the contents of which are herein incorporated by reference in its entirety. The nanoemulsions may comprise nanoparticles described herein. As a non-limiting example, the nanoparticles may comprise a liquid hydrophobic core which may be surrounded or coated with a lipid or surfactant layer. The lipid or surfactant layer may comprise at least one membrane-integrating peptide and may also comprise a targeting ligand (see e.g., U.S. Pat. No. 8,496,945, the contents of which are herein incorporated by reference in its entirety).

### Cations and Anions

Formulations of antibody compositions disclosed herein may include cations or anions. In one embodiment, the formulations include metal cations such as, but not limited to, Zn2+, Ca2+, Cu2+, Mg+ and combinations thereof. As a non-limiting example, formulations may include polymers and an antibody composition complexed with a metal cation (See e.g., U.S. Pat. Nos. 6,265,389 and 6,555,525, each of which is herein incorporated by reference in its entirety).

In some embodiments, cationic nanoparticles comprising combinations of divalent and monovalent cations may be formulated with antibody compositions. Such nanoparticles may form spontaneously in solution over a give period (e.g. hours, days, etc). Such nanoparticles do not form in the presence of divalent cations alone or in the presence of monovalent cations alone. The delivery of antibody compositions in cationic nanoparticles or in one or more depot comprising cationic nanoparticles may improve antibody composition bioavailability by acting as a long-acting depot and/or reducing the rate of degradation by nucleases.

### Molded Nanoparticles and Microparticles

The antibody compositions disclosed herein may be formulated in nanoparticles and/or microparticles. These nanoparticles and/or microparticles may be molded into any size shape and chemistry. As an example, the nanoparticles and/or microparticles may be made using the PRINT® technology by LIQUIDA TECHNOLOGIES® (Morrisville, N.C.) (See e.g., International Pub. No. WO2007024323; the contents of which are herein incorporated by reference in its entirety).

In one embodiment, the molded nanoparticles may comprise a core of the antibody compositions disclosed herein and a polymer shell. The polymer shell may be any of the polymers described herein and are known in the art. In an additional embodiment, the polymer shell may be used to protect the antibody compositions in the core.

In one embodiment, the antibody compositions of the present invention may be formulated in microparticles. The microparticles may contain a core of the antibody compositions and a cortext of a biocompatible and/or biodegradable polymer. As a non-limiting example, the microparticles which may be used with the present invention may be those described in U.S. Pat. No. 8,460,709, U.S. Patent Publication No. US20130129830 and International Patent Publication No WO2013075068, each of which is herein incorporated by reference in its entirety. As another non-limiting example, the microparticles may be designed to extend the release of the antibody compositions of the present invention over a desired period of time (see e.g, extended release of a therapeutic protein in U.S. Patent Publication No. US20130129830, herein incorporated by reference in its entirety).

The microparticle for use with the present invention may have a diameter of at least 1 micron to at least 100 microns (e.g., at least 1 micron, at least 5 micron, at least 10 micron, at least 15 micron, at least 20 micron, at least 25 micron, at least 30 micron, at least 35 micron, at least 40 micron, at least 45 micron, at least 50 micron, at least 55 micron, at least 60 micron, at least 65 micron, at least 70 micron, at least 75 micron, at least 80 micron, at least 85 micron, at least 90 micron, at least 95 micron, at least 97 micron, at least 99 micron, and at least 100 micron).

### NanoJackets and NanoLiposomes

The p antibody compositions disclosed herein may be formulated in NanoJackets and NanoLiposomes by Keystone Nano (State College, Pa.). NanoJackets are made of compounds that are naturally found in the body including calcium, phosphate and may also include a small amount of silicates. Nanojackets may range in size from 5 to 50 nm and may be used to deliver hydrophilic and hydrophobic compounds such as, but not limited to, antibody compositions.

NanoLiposomes are made of lipids such as, but not limited to, lipids which naturally occur in the body. NanoLiposomes may range in size from 60-80 nm and may be used to deliver hydrophilic and hydrophobic compounds such as, but not limited to, antibody compositions. In one aspect, the antibody compositions disclosed herein are formulated in a NanoLiposome such as, but not limited to, Ceramide NanoLiposomes.

### Pseudovirions

In one embodiment, the antibody compositions disclosed herein may be formulated in Pseudovirions (e.g., pseudo-virions). As a non-limiting example, the pseudovirions may be those developed and/or are described by Aura Biosciences (Cambridge, Mass.). In one aspect, the pseudovirion may be developed to deliver drugs to keratinocytes and basal membranes (See e.g., US Patent Publication Nos. US20130012450, US20130012566, US21030012426 and US20120207840 and International Publication No. WO2013009717, each of which is herein incorporated by reference in its entirety).

In one embodiment, the pseudovirion used for delivering the antibody compositions of the present invention may be derived from viruses such as, but not limited to, herpes and papillomaviruses (See e.g., US Patent Publication Nos. US Patent Publication Nos. US20130012450, US20130012566, US21030012426 and US20120207840 and International Publication No. WO2013009717, each of which is herein incorporated by reference in its entirety; and Ma et al. HPV pseudovirions as DNA delivery vehicles. Ther Deliv. 2011: 2(4): 427-430; Kines et al. The initial steps leading to papillomavirus infection occur on the basement membrane prior to cell surface binding. PNAS 2009:106(48), 20458-20463; Roberts et al. Genital transmission of HPV in a mouse model is potentiated by nonoxynol-9 and inhibited by carrageenan. Nature Medicine. 2007:13(7) 857-861; Gordon et al., Targeting the Vaginal Mucosa with Human Papillomavirus Pseudovirion Vaccines delivering SIV DNA. J Immunol. 2012 188(2) 714-723; Cuburu et al., Intravaginal immunization with HPV vectors induces tissue-resident CD8+ T cell responses. The Journal of Clinical Investigation. 2012: 122(12) 4606-4620; Hung et al., Ovarian Cancer Gene Therapy Using HPV-16 Pseudovirion Carrying the HSV-tk Gene. PLoS ONE. 2012: 7(7) e40983; Johnson et al., Role of Heparan Sulfate in Attachment to and Infection of the Murine Female Genital Tract by Human Papillomavirus. J Virology. 2009: 83(5) 2067-2074; each of which is herein incorporated by reference in its entirety).

The pseudovirion may be a virus-like particle (VLP) prepared by the methods described in US Patent Publication No. US20120015899 and US20130177587 and International Patent Publication No. WO2010047839 WO2013116656, WO2013106525 and WO2013122262, the contents of each of which is herein incorporated by reference in its entirety. In one aspect, the VLP may be, but is not limited to, bacteriophages MS, Qβ, R17, fr, GA, Sp, MI, I, MXI, NL95, AP205, f2, PP7, and the plant viruses Turnip crinkle virus (TCV), Tomato bushy stunt virus (TBSV), Southern bean mosaic virus (SBMV) and members of the genus Bromovirus including Broad bean mottle virus, Brome mosaic virus, Cassia yellow blotch virus, Cowpea chlorotic mottle virus (CCMV), Melandrium yellow fleck virus, and Spring beauty latent virus. In another aspect, the VLP may be derived from the influenza virus as described in US Patent Publication No. US20130177587 or U.S. Pat. No. 8,506,967, the contents of each of which are herein incorporated by reference in its entirety. In yet another aspect, the VLP may comprise a B7-1 and/or B7-2 molecule anchored to a lipid membrane or the exterior of the particle such as described in International Patent Publication No. WO2013116656, the contents of which are herein incorporated by reference in its entirety. In one aspect, the VLP may be derived from norovirus, rotavirus recombinant VP6 protein or double layered VP2/VP6 such as the VLP described in International Patent Publication No. WO2012049366, the contents of which are herein incorporated by reference in its entirety.

The pseudovirion may be a human papilloma virus-like particle such as, but not limited to, those described in International Publication No. WO2010120266 and US Patent Publication No. US20120171290, each of which is herein incorporated by reference in its entirety and Ma et al. HPV pseudovirions as DNA delivery vehicles. Ther Deliv. 2011: 2(4): 427-430; Kines et al. The initial steps leading to papillomavirus infection occur on the basement membrane prior to cell surface binding. PNAS 2009:106(48), 20458-20463; Roberts et al. Genital transmission of HPV in a mouse model is potentiated by nonoxynol-9 and inhibited by carrageenan. Nature Medicine. 2007:13(7) 857-861; Gordon et al., Targeting the Vaginal Mucosa with Human Papillomavirus Pseudovirion Vaccines delivering SIV DNA. J Immunol. 2012 188(2) 714-723; Cuburu et al., Intravaginal immunization with HPV vectors induces tissue-resident CD8+ T cell responses. The Journal of Clinical Investigation. 2012: 122(12) 4606-4620; Hung et al., Ovarian Cancer Gene Therapy Using HPV-16 Pseudovirion Carrying the HSV-tk Gene. PLoS ONE. 2012: 7(7) e40983; Johnson et al., Role of Heparan Sulfate in Attachment to and Infection of the Murine Female Genital Tract by Human Papillomavirus. J Virology. 2009: 83(5) 2067-2074; each of which is herein incorporated by reference in its entirety.

In one aspect, the pseudovirions may be virion derived nanoparticles such as, but not limited to, those described in US Patent Publication No. US20130116408 and US20130115247, each of which is herein incorporated by reference in their entirety. As a non-limiting example, the virion derived nanoparticles may be used to deliver antibody compositions which may be used in the treatment for cancer and/or enhance the immune system's recognition of the tumor. As a non-limiting example, the virion-derived nanoparticle which may selectively deliver an agent to at least one tumor may be the papilloma-derived particles described in International Patent Publication No. WO2013119877, the contents of which are herein incorporated by reference in its entirety. The virion derived nanoparticles may be made by the methods described in US Patent Publication No. US20130116408 and US20130115247 or International Patent Publication No. WO2013119877, each of which is herein incorporated by reference in their entirety.

In one embodiment, the virus-like particle (VLP) may be a self-assembled particle. Non-limiting examples of self-assembled VLPs and methods of making the self-assembled VLPs are described in International Patent Publication No. WO2013122262, the contents of which are herein incorporated by reference in its entirety.

### Minicells

In one aspect, the antibody compositions may be formulated in bacterial minicells. As a non-limiting example, bacterial minicells may be those described in International Publication No. WO2013088250 or US Patent Publication No. US20130177499, the contents of each of which are herein incorporated by reference in its entirety. The bacterial minicells comprising therapeutic agents such as antibody compositions described herein may be used to deliver the therapeutic agents to brain tumors.

### Semi-Solid Compositions

In one embodiment, the antibody compositions may be formulated with a hydrophobic matrix to form a semi-solid composition. As a non-limiting example, the semi-solid composition or paste-like composition may be made by the methods described in International Patent Publication No WO201307604, herein incorporated by reference in its entirety. The semi-solid composition may be a sustained release formulation as described in International Patent Publication No WO201307604, herein incorporated by reference in its entirety.

In another embodiment, the semi-solid composition may further have a micro-porous membrane or a biodegradable polymer formed around the composition (see e.g., International Patent Publication No WO201307604, herein incorporated by reference in its entirety).

The semi-solid composition using the antibody compositions of the present invention may have the characteristics of the semi-solid mixture as described in International Patent Publication No WO201307604, herein incorporated by reference in its entirety (e.g., a modulus of elasticity of at least 10⁻⁴ N·mm⁻², and/or a viscosity of at least 100 mPa·s).

### Exosomes

In one embodiment, the antibody compositions may be formulated in exosomes. The exosomes may be loaded with at least one antibody composition and delivered to cells, tissues and/or organisms. As a non-limiting example, the antibody compositions may be loaded in the exosomes described in International Publication No. WO2013084000, herein incorporated by reference in its entirety.

### Silk-Based Delivery

In one embodiment, the antibody compositions may be formulated in a sustained release silk-based delivery system. The silk-based delivery system may be formed by contacting a silk fibroin solution with a therapeutic agent such as, but not limited to, the antibody compositions described herein and/or known in the art. As a non-limiting example, the sustained release silk-based delivery system which may be used in the present invention and methods of making such system are described in US Patent Publication No. US20130177611, the contents of which are herein incorporated by reference in its entirety.

### Microparticles

In one embodiment, formulations comprising antibody compositions may comprise microparticles. The microparticles may comprise a polymer described herein and/or known in the art such as, but not limited to, poly(α-hydroxy acid), a polyhydroxy butyric acid, a polycaprolactone, a polyorthoester and a polyanhydride. The microparticle may have adsorbent surfaces to adsorb biologically active molecules such as antibody compositions. As a non-limiting example microparticles for use with the present invention and methods of making microparticles are described in US Patent Publication No. US2013195923 and US20130195898 and U.S. Pat. Nos. 8,309,139 and 8,206,749, the contents of each of which are herein incorporated by reference in its entirety.

In another embodiment, the formulation may be a microemulsion comprising microparticles and antibody compositions. As a non-limiting example, microemulsions comprising microparticles are described in US Patent Publication No. US2013195923 and US20130195898 and U.S. Pat. Nos. 8,309,139 and 8,206,749, the contents of each of which are herein incorporated by reference in its entirety.

### Amino Acid Lipids

In one embodiment, the antibody compositions may be formulated in amino acid lipids. Amino acid lipids are lipophilic compounds comprising an amino acid residue and one or more lipophilic tails. Non-limiting examples of amino acid lipids and methods of making amino acid lipids are described in U.S. Pat. No. 8,501,824, the contents of which are herein incorporated by reference in its entirety.

In one embodiment, the amino acid lipids have a hydrophilic portion and a lipophilic portion. The hydrophilic portion may be an amino acid residue and a lipophilic portion may comprise at least one lipophilic tail.

In one embodiment, the amino acid lipid formulations may be used to deliver the antibody compositions to a subject.

In another embodiment, the amino acid lipid formulations may deliver an antibody composition in releasable form which comprises an amino acid lipid that binds and releases the antibody composition. As a non-limiting example, the release of the antibody compositions may be provided by an acid-labile linker such as, but not limited to, those described in U.S. Pat. Nos. 7,098,032, 6,897,196, 6,426,086, 7,138,382, 5,563,250, and 5,505,931, the contents of each of which are herein incorporated by reference in its entirety.

### Microvesicles

In one embodiment, antibody compositions may be formulated in microvesicles. Non-limiting examples of microvesicles include those described in US Patent Publication No. US20130209544, the contents of which are herein incorporated by reference in its entirety.

In one embodiment, the microvesicles is an ARRDC1-mediated microvesicles (ARMMs). Non-limiting examples of ARMMs and methods of making ARMMs are described in International Patent Publication No. WO2013119602, the contents of which are herein incorporated by reference in its entirety.

### Interpolyelectrolyte Complexes

In one embodiment, the antibody compositions may be formulated in an interpolyelectrolyte complex. Interpolyelectrolyte complexes are formed when charge-dynamic polymers are complexed with one or more anionic molecules. Non-limiting examples of charge-dynamic polymers and interpolyelectrolyte complexes and methods of making interpolyelectrolyte complexes are described in U.S. Pat. No. 8,524,368, the contents of which is herein incorporated by reference in its entirety.

### Crystalline Polymeric Systems

In one embodiment, the antibody compositions may be formulated in crystalline polymeric systems. Crystalline polymeric systems are polymers with crystalline moieties and/or terminal units comprising crystalline moieties. Non-limiting examples of polymers with crystalline moieties and/or terminal units comprising crystalline moieties termed “CYC polymers,” crystalline polymer systems and methods of making such polymers and systems are described in U.S. Pat. No. 8,524,259, the contents of which are herein incorporated by reference in its entirety.

### Excipients

Antibody pharmaceutical formulations may additionally comprise a pharmaceutically acceptable excipient, which, as used herein, includes, but are not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, antioxidants, osmolality adjusting agents, pH adjusting agents and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro (Lippincott, Williams & Wilkins, Baltimore, Md., 2006; incorporated herein by reference in its entirety). The use of a conventional excipient medium may be contemplated within the scope of the present disclosure, except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER® 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); amino acids (e.g., glycine); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate)(VEEGUM®, and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Oxidation is a potential degradation pathway for mRNA, especially for liquid mRNA formulations. In order to prevent oxidation, antioxidants can be added to the formulation. Exemplary antioxidants include, but are not limited to, alpha tocopherol, ascorbic acid, acorbyl palmitate, benzyl alcohol, butylated hydroxyanisole, EDTA, m-cresol, methionine, butylated hydroxytoluene, monothioglycerol, potassium metabisulfite, propionic acid, propyl gallate, sodium ascorbate, sodium bisulfite, sodium metabisulfite, thioglycerol and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

In some embodiments, the pH of antibody composition solutions are maintained between pH 5 and pH 8 to improve stability. Exemplary buffers to control pH may include, but are not limited to sodium phosphate, sodium citrate, sodium succinate, histidine (or histidine-HCl), sodium carbonate, and/or sodium malate. In another embodiment, the exemplary buffers listed above may be used with additional monovalent counterions (including, but not limited to potassium). Divalent cations may also be used as buffer counterions; however, these are not preferred due to complex formation and/or mRNA degradation.

Exemplary buffering agents may also include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

Exemplary additives include physiologically biocompatible buffers (e.g., trimethylamine hydrochloride), addition of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). In addition, antioxidants and suspending agents can be used.

### Cryoprotectants

In some embodiments, formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with antibody compositions in order to stabilize them during freezing. Frozen storage of mRNA between −20° C. and −80° C. may be advantageous for long term (e.g. 36 months) stability of polynucleotide. In some embodiments, cryoprotectants are included in formulations to stabilize polynucleotide through freeze/thaw cycles and under frozen storage conditions. Cryoprotectants of the present invention may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

### Bulking Agents

In some embodiments, formulations may comprise bulking agents. As used herein, ther term “bulking agent” refers to one or more agents included in formulations to impart a desired consistency to the formulation and/or stabilization of formulation components. In some embodiments, bulking agents are included in lyophilized formulations to yield a “pharmaceutically elegant” cake, stabilizing the lyophilized antibody compositions during long term (e.g. 36 month) storage. Bulking agents of the present invention may include, but are not limited to sucrose, trehalose, mannitol, glycine, lactose and/or raffinose. In some embodiments, combinations of cryoprotectants and bulking agents (for example, sucrose/glycine or trehalose/mannitol) may be included to both stabilize antibody compositions during freezing and provide a bulking agent for lyophilization.

Non-limiting examples of formulations and methods for formulating the antibody compositions of the present invention are also provided in International Publication No WO2013090648 filed Dec. 14, 2012, the contents of which are incorporated herein by reference in their entirety.

### Inactive Ingredients

In some embodiments, antibody compositions may comprise at least one excipient which is an inactive ingredient. As used herein, ther term “inactive ingredient” refers to one or more inactive agents included in formulations. In some embodiments, all, none or some of the inactive ingredients which may be used in the formulations of the present invention may be approved by the US Food and Drug Administration (FDA). A non-exhaustive list of inactive ingredients and the routes of administration the inactive ingredients may be formulated in are described in Table 9 of copending application U.S. 61/912,635 filed Dec. 6, 2013 (Attorney Docket Number M073.60), the contents of which are incorporated herein by reference.

### Delivery

The present disclosure encompasses the delivery of antibody compositions for any of therapeutic, pharmaceutical, diagnostic or imaging by any appropriate route taking into consideration likely advances in the sciences of drug delivery. Delivery may be naked or formulated.

### Naked Delivery

The antibody compositions of the present invention may be delivered to a cell naked. As used herein in, “naked” refers to delivering antibody compositions free from agents which promote transfection. For example, the antibody compositions delivered to the cell may contain no modifications. The naked antibody compositions may be delivered to the cell using routes of administration known in the art and described herein.

### Formulated Delivery

The antibody compositions of the present invention may be formulated, using the methods described herein. The formulations may contain polynucleotides which may be modified and/or unmodified. The formulations may further include, but are not limited to, cell penetration agents, a pharmaceutically acceptable carrier, a delivery agent, a bioerodible or biocompatible polymer, a solvent, and a sustained-release delivery depot. The formulated antibody compositions may be delivered to the cell using routes of administration known in the art and described herein.

The antibody compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like.

### Administration

The antibody compositions of the present invention may be administered by any route which results in a therapeutically effective outcome. These include, but are not limited to enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier. In one embodiment, a formulation for a route of administration may include at least one inactive ingredient.

Non-limiting examples of routes of administration and inactive ingredients which may be included in formulations for the specific route of administration is shown in Table 10 of copending application U.S. 61/912,635 filed Dec. 6, 2013 (Attorney Docket Number M073.60), the contents of which are incorporated herein by reference.

Non-limiting routes of administration for the antibody compositions of the present invention are described below.

### Parenteral and Injectable Administration

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

A pharmaceutical composition for parenteral administration may comprise at least one inactive ingredient. Any or none of the inactive ingredients used may have been approved by the US Food and Drug Administration (FDA). A non-exhaustive list of inactive ingredients for use in pharmaceutical compositions for parenteral administration includes hydrochloric acid, mannitol, nitrogen, sodium acetate, sodium chloride and sodium hydroxide.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. The sterile formulation may also comprise adjuvants such as local anesthetics, preservatives and buffering agents.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Injectable formulations may be for direct injection into a region of a tissue, organ and/or subject. As a non-limiting example, a tissue, organ and/or subject may be directly injected a formulation by intramyocardial injection into the ischemic region. (See e.g., Zangi et al. Nature Biotechnology 2013; the contents of which are herein incorporated by reference in its entirety).

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

Compositions for rectal or vaginal (e.g., transvaginal) administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

As a non-limiting example, the formulations for rectal and/or vaginal administration may be prepared by mixing the drug with a suitable non-irritating excipient that is solid at ordinary temperatures but liquid at the rectal temperature and will therefore melt in the rectum and/or vagina to release the drug. Such materials include cocoa butter and polyethylene glycols.

A pharmaceutical composition for rectal administration may comprise at least one inactive ingredient. Any or none of the inactive ingredients used may have been approved by the US Food and Drug Administration (FDA). A non-exhaustive list of inactive ingredients for use in pharmaceutical compositions for rectal administration includes alcohol, alcohol, dehydrated, aluminum subacetate, anhydrous citric acid, aniseed oil, ascorbic acid, ascorbyl palmitate, balsam peru, benzoic acid, benzyl alcohol, bismuth subgallate, butylated hydroxyanisole, butylated hydroxytoluene, butylparaben, caramel, carbomer 934, carbomer 934p, carboxypolymethylene, cerasynt-se, cetyl alcohol, cocoa butter, coconut oil, hydrogenated, coconut oil/palm kernel oil glycerides, hydrogenated, cola nitida seed extract, d&c yellow no. 10, dichlorodifluoromethane, dichlorotetrafluoroethane, dimethyldioctadecylammonium bentonite, edetate calcium disodium, edetate disodium, edetic acid, epilactose, ethylenediamine, fat, edible, fat, hard, fd&c blue no. 1, fd&c green no. 3, fd&c yellow no. 6, flavor FIG. 827118, flavor raspberry pfc-8407, fructose, galactose, glycerin, glyceryl palmitate, glyceryl stearate, glyceryl stearate/peg stearate, glyceryl stearate/peg-40 stearate, glycine, hydrocarbon, hydrochloric acid, hydrogenated palm oil, hypromelloses, lactose, lanolin, lecithin, light mineral oil, magnesium aluminum silicate, magnesium aluminum silicate hydrate, methylparaben, nitrogen, palm kernel oil, paraffin, petrolatum, white, polyethylene glycol 1000, polyethylene glycol 1540, polyethylene glycol 3350, polyethylene glycol 400, polyethylene glycol 4000, polyethylene glycol 6000, polyethylene glycol 8000, polysorbate 60, polysorbate 80, potassium acetate, potassium metabisulfite, propylene glycol, propylparaben, saccharin sodium, saccharin sodium anhydrous, silicon dioxide, colloidal, simethicone, sodium benzoate, sodium carbonate, sodium chloride, sodium citrate, sodium hydroxide, sodium metabisulfite, sorbitan monooleate, sorbitan sesquioleate, sorbitol, sorbitol solution, starch, steareth-10, steareth-40, sucrose, tagatose, d-, tartaric acid, dl-, trolamine, tromethamine, vegetable oil glyceride, hydrogenated, vegetable oil, hydrogenated, wax, emulsifying, white wax, xanthan gum and zinc oxide.

A pharmaceutical composition for vaginal administration may comprise at least one inactive ingredient. Any or none of the inactive ingredients used may have been approved by the US Food and Drug Administration (FDA). A non-exhaustive list of inactive ingredients for use in pharmaceutical compositions for vaginal administration includes adipic acid, alcohol, denatured, allantoin, anhydrous lactose, apricot kernel oil peg-6 esters, barium sulfate, beeswax, bentonite, benzoic acid, benzyl alcohol, butylated hydroxyanisole, butylated hydroxytoluene, calcium lactate, carbomer 934, carbomer 934p, cellulose, microcrystalline, ceteth-20, cetostearyl alcohol, cetyl alcohol, cetyl esters wax, cetyl palmitate, cholesterol, choleth, citric acid, citric acid monohydrate, coconut oil/palm kernel oil glycerides, hydrogenated, crospovidone, edetate disodium, ethylcelluloses, ethylene-vinyl acetate copolymer (28% vinyl acetate), ethylene-vinyl acetate copolymer (9% vinylacetate), fatty alcohols, fd&c yellow no. 5, gelatin, glutamic acid, dl-, glycerin, glyceryl isostearate, glyceryl monostearate, glyceryl stearate, guar gum, high density polyethylene, hydrogel polymer, hydrogenated palm oil, hypromellose 2208 (15000 mpa·s), hypromelloses, isopropyl myristate, lactic acid, lactic acid, dl-, lactose, lactose monohydrate, lactose, hydrous, lanolin, lanolin anhydrous, lecithin, lecithin, soybean, light mineral oil, magnesium aluminum silicate, magnesium aluminum silicate hydrate, magnesium stearate, methyl stearate, methylparaben, microcrystalline wax, mineral oil, nitric acid, octyldodecanol, peanut oil, peg 6-32 stearate/glycol stearate, peg-100 stearate, peg-120 glyceryl stearate, peg-2 stearate, peg-5 oleate, pegoxol 7 stearate, petrolatum, white, phenylmercuric acetate, phospholipon 90 g, phosphoric acid, piperazine hexahydrate, poly(dimethylsiloxane/methylvinylsiloxane/methylhydrogensiloxane) dimethylvinyl or dimethylhydroxy or trimethyl endblocked, polycarbophil, polyester, polyethylene glycol 1000, polyethylene glycol 3350, polyethylene glycol 400, polyethylene glycol 4000, polyethylene glycol 6000, polyethylene glycol 8000, polyglyceryl-3 oleate, polyglyceryl-4 oleate, polyoxyl palmitate, polysorbate 20, polysorbate 60, polysorbate 80, polyurethane, potassium alum, potassium hydroxide, povidone k29/32, povidones, promulgen d, propylene glycol, propylene glycol monopalmitostearate, propylparaben, quaternium-15 cis-form, silicon dioxide, silicon dioxide, colloidal, silicone, sodium bicarbonate, sodium citrate, sodium hydroxide, sodium lauryl sulfate, sodium metabisulfite, sodium phosphate, dibasic, anhydrous, sodium phosphate, monobasic, anhydrous, sorbic acid, sorbitan monostearate, sorbitol, sorbitol solution, spermaceti, stannous 2-ethylhexanoate, starch, starch 1500, pregelatinized, starch, corn, stearamidoethyl diethylamine, stearic acid, stearyl alcohol, tartaric acid, dl-, tert-butylhydroquinone, tetrapropyl orthosilicate, trolamine, urea, vegetable oil, hydrogenated, wecobee fs, white ceresin wax and white wax.

### Oral Administration

Liquid dosage forms for oral administration include, but are not limited to, pharmaceutically acceptable emulsions, microemulsions, solutions, suspensions, syrups, and/or elixirs. In addition to active ingredients, liquid dosage forms may comprise inert diluents and/or excipients commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

Syrups and elixirs can be formulated with sweetening agents, for example glycerol, propylene glycol, sorbitol, glucose or sucrose. Such formulations can also contain a demulcent, a preservative, flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

Suspensions for oral dosage may contain the active materials in a mixture with excipients suitable for the manufacture of aqueous suspensions. Such excipients may be suspending agents, as a non-limiting example the suspending agents may be sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate; or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions for oral dosage can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid

The oral dosage may also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents. The solid dosage forms may also dissolve once they come in contact with liquid such as, but not limited to, salvia and bile.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations.

Solid dosage forms may be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Dosage forms for oral delivery may also be chewable or may be suckable (e.g., lozenge form). The chewable dosages forms may be sustained release formulations such as, but not limited to, the sustained release compositions described in International Publication No WO2013082470 and US Publication No US20130142876, each of which is herein incorporated by reference in its entirety. The chewable dosage forms may comprise amphipathic lipids such as, but not limited to, those described in International Publication No WO2013082470 and US Publication No US20130142876, each of which is herein incorporated by reference in its entirety.

### Topical or Transdermal Administration

As described herein, compositions containing the antibody compositions of the invention may be formulated for administration topically and/or transdermally. The skin may be an ideal target site for delivery as it is readily accessible. Gene expression may be restricted not only to the skin, potentially avoiding nonspecific toxicity, but also to specific layers and cell types within the skin.

The site of cutaneous expression of the delivered compositions will depend on the route of nucleic acid delivery. Three routes are commonly considered to deliver antibody compositions to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). Antibody compositions can be delivered to the skin by several different approaches known in the art. Most topical delivery approaches have been shown to work for delivery of DNA, such as but not limited to, topical application of non-cationic liposome-DNA complex, cationic liposome-DNA complex, particle-mediated (gene gun), puncture-mediated gene transfections, and viral delivery approaches. After delivery of the nucleic acid, gene products have been detected in a number of different skin cell types, including, but not limited to, basal keratinocytes, sebaceous gland cells, dermal fibroblasts and dermal macrophages.

Ointments, creams and gels for topical administration, can, for example, can be formulated with an aqueous or oily base with the addition of suitable thickening and/or gelling agent and/or solvents. Non limiting examples of such bases can thus, for example, include water and/or an oil such as liquid paraffin or a vegetable oil such as arachis oil or castor oil, or a solvent such as polyethylene glycol. Various thickening agents and gelling agents can be used depending on the nature of the base. Non-limiting examples of such agents include soft paraffin, aluminum stearate, cetostearyl alcohol, polyethylene glycols, woolfat, beeswax, carboxypolymethylene and cellulose derivatives, and/or glyceryl monostearate and/or non-ionic emulsifying agents.

Lotions for topical administration may be formulated with an aqueous or oily base and will in general also contain one or more emulsifying agents, stabilizing agents, dispersing agents, suspending agents or thickening agents.

In one embodiment, the invention provides for a variety of dressings (e.g., wound dressings) or bandages (e.g., adhesive bandages) for conveniently and/or effectively carrying out methods of the present invention. Typically dressing or bandages may comprise sufficient amounts of pharmaceutical compositions and/or polynucleotides described herein to allow a user to perform multiple treatments of a subject(s).

In one embodiment, the invention provides for the antibody compositions to be delivered in more than one injection.

In one embodiment, before topical and/or transdermal administration at least one area of tissue, such as skin, may be subjected to a device and/or solution which may increase permeability. In one embodiment, the tissue may be subjected to an abrasion device to increase the permeability of the skin (see U.S. Patent Publication No. 20080275468, herein incorporated by reference in its entirety). In another embodiment, the tissue may be subjected to an ultrasound enhancement device. An ultrasound enhancement device may include, but is not limited to, the devices described in U.S. Publication No. 20040236268 and U.S. Pat. Nos. 6,491,657 and 6,234,990; each of which are herein incorporated by reference in their entireties. Methods of enhancing the permeability of tissue are described in U.S. Publication Nos. 20040171980 and 20040236268 and U.S. Pat. No. 6,190,315; each of which are herein incorporated by reference in their entireties.

In one embodiment, a device may be used to increase permeability of tissue before delivering formulations of modified mRNA described herein. The permeability of skin may be measured by methods known in the art and/or described in U.S. Pat. No. 6,190,315, herein incorporated by reference in its entirety. As a non-limiting example, a modified mRNA formulation may be delivered by the drug delivery methods described in U.S. Pat. No. 6,190,315, herein incorporated by reference in its entirety.

In another non-limiting example tissue may be treated with a eutectic mixture of local anesthetics (EMLA) cream before, during and/or after the tissue may be subjected to a device which may increase permeability. Katz et al. (Anesth Analg (2004); 98:371-76; herein incorporated by reference in its entirety) showed that using the EMLA cream in combination with a low energy, an onset of superficial cutaneous analgesia was seen as fast as 5 minutes after a pretreatment with a low energy ultrasound.

In one embodiment, enhancers may be applied to the tissue before, during, and/or after the tissue has been treated to increase permeability. Enhancers include, but are not limited to, transport enhancers, physical enhancers, and cavitation enhancers. Non-limiting examples of enhancers are described in U.S. Pat. No. 6,190,315, herein incorporated by reference in its entirety.

In one embodiment, a device may be used to increase permeability of tissue before delivering formulations of antibody compositions described herein, which may further contain a substance that invokes an immune response. In another non-limiting example, a formulation containing a substance to invoke an immune response may be delivered by the methods described in U.S. Publication Nos. 20040171980 and 20040236268; each of which are herein incorporated by reference in their entireties.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required.

Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 0.1% to about 10% (w/w) active ingredient, although the concentration of active ingredient may be as high as the solubility limit of the active ingredient in the solvent. Formulations for topical administration may further comprise one or more of the additional ingredients described herein.

A pharmaceutical composition for topical administration may comprise at least one inactive ingredient. Any or none of the inactive ingredients used may have been approved by the US Food and Drug Administration (FDA). A non-exhaustive list of inactive ingredients for use in pharmaceutical compositions for topical administration includes alpha-terpineol, alpha-tocopherol, alpha-tocopherol acetate, DL-, alpha-tocopherol, DL-, 1,2,6-hexanetriol, 1-O-tolylbiguanide, 2-ethyl-1,6-hexanediol, acetic acid, acetone, acetylated lanolin alcohols, acrylates copolymer, adhesive tape, alcohol, alcohol, dehydrated, alcohol, denatured, alcohol, diluted, alkyl ammonium sulfonic acid betaine, alkyl aryl sodium sulfonate, allantoin, almond oil, aluminum acetate, aluminum chlorhydroxy allantoinate, aluminum hydroxide, aluminum hydroxide-sucrose, hydrated, aluminum hydroxide gel, aluminum hydroxide gel F 500, aluminum hydroxide gel F 5000, aluminum monostearate, aluminum oxide, aluminum silicate, aluminum starch octenylsuccinate, aluminum stearate, aluminum sulfate anhydrous, amerchol c, amerchol-cab, aminomethylpropanol, ammonia solution, ammonia solution, strong, ammonium hydroxide, ammonium lauryl sulfate, ammonium nonoxynol-4 sulfate, ammonium salt of c-12-c-15 linear primary alcohol ethoxylate, ammonyx, amphoteric-2, amphoteric-9, anhydrous citric acid, anhydrous trisodium citrate, anoxid sbn, antifoam, apricot kernel oil peg-6 esters, aquaphor, arlacel, ascorbic acid, ascorbyl palmitate, beeswax, beeswax, synthetic, beheneth-10, bentonite, benzalkonium chloride, benzoic acid, benzyl alcohol, betadex, boric acid, butane, butyl alcohol, butyl ester of vinyl methyl ether/maleic anhydride copolymer (125000 mw), butyl stearate, butylated hydroxyanisole, butylated hydroxytoluene, butylene glycol, butylparaben, c20-40 pareth-24, calcium chloride, calcium hydroxide, canada balsam, caprylic/capric triglyceride, caprylic/capric/stearic triglyceride, captan, caramel, carbomer 1342, carbomer 1382, carbomer 934, carbomer 934p, carbomer 940, carbomer 941, carbomer 980, carbomer 981, carbomer homopolymer type b (allyl pentaerythritol crosslinked), carbomer homopolymer type c (allyl pentaerythritol crosslinked), carboxy vinyl copolymer, carboxymethylcellulose, carboxymethylcellulose sodium, carboxypolymethylene, carrageenan, carrageenan salt, castor oil, cedar leaf oil, cellulose, cerasynt-se, ceresin, ceteareth-12, ceteareth-15, ceteareth-30, cetearyl alcohol/ceteareth-20, cetearyl ethylhexanoate, ceteth-10, ceteth-2, ceteth-20, ceteth-23, cetostearyl alcohol, cetrimonium chloride, cetyl alcohol, cetyl esters wax, cetyl palmitate, chlorobutanol, chlorocresol, chloroxylenol, cholesterol, choleth-24, citric acid, citric acid monohydrate, cocamide ether sulfate, cocamine oxide, coco betaine, coco diethanolamide, coco monoethanolamide, cocoa butter, coco-glycerides, coconut oil, cocoyl caprylocaprate, collagen, coloring suspension, cream base, creatinine, crospovidone, cyclomethicone, cyclomethicone/dimethicone copolyol, d&c red no. 28, d&c red no. 33, d&c red no. 36, d&c red no. 39, d&c yellow no. 10, decyl methyl sulfoxide, dehydag wax sx, dehydroacetic acid, dehymulse, denatonium benzoate, dextrin, diazolidinyl urea, dichlorobenzyl alcohol, dichlorodifluoromethane, dichlorotetrafluoroethane, diethanolamine, diethyl sebacate, diethylene glycol monoethyl ether, dihydroxyaluminum aminoacetate, diisopropanolamine, diisopropyl adipate, diisopropyl dilinoleate, dimethicone 350, dimethicone copolyol, dimethicone medical fluid 360, dimethyl isosorbide, dimethyl sulfoxide, dinoseb ammonium salt, disodium cocoamphodiacetate, disodium laureth sulfosuccinate, disodium lauryl sulfosuccinate, dmdm hydantoin, docosanol, docusate sodium, edetate disodium, edetate sodium, edetic acid, entsufon, entsufon sodium, epitetracycline hydrochloride, essence bouquet 9200, ethyl acetate, ethylcelluloses, ethylene glycol, ethylenediamine, ethylenediamine dihydrochloride, ethylhexyl hydroxystearate, ethylparaben, fatty acid pentaerythriol ester, fatty acids, fatty alcohol citrate, fd&c blue no. 1, fd&c red no. 4, fd&c red no. 40, fd&c yellow no. 10 (delisted), fd&c yellow no. 5, fd&c yellow no. 6, ferric oxide, flavor rhodia pharmaceutical no. rf 451, formaldehyde, formaldehyde solution, fractionated coconut oil, fragrance 3949-5, fragrance 520a, fragrance 6.007, fragrance 91-122, fragrance 9128-y, fragrance 93498 g, fragrance balsam pine no. 5124, fragrance bouquet 10328, fragrance chemoderm 6401-b, fragrance chemoderm 6411, fragrance cream no. 73457, fragrance cs-28197, fragrance felton 066m, fragrance firmenich 47373, fragrance givaudan ess 9090/1c, fragrance h-6540, fragrance herbal 10396, fragrance nj-1085, fragrance p o fl-147, fragrance pa 52805, fragrance pera derm d, fragrance rbd-9819, fragrance shaw mudge u-7776, fragrance tf 044078, fragrance ungerer honeysuckle k 2771, fragrance ungerer n5195, gelatin, gluconolactone, glycerin, glyceryl citrate, glyceryl isostearate, glyceryl monostearate, glyceryl oleate, glyceryl oleate/propylene glycol, glyceryl palmitate, glyceryl ricinoleate, glyceryl stearate, glyceryl stearate-laureth-23, glyceryl stearate/peg-100 stearate, glyceryl stearate-stearamidoethyl diethylamine, glycol distearate, glycol stearate, guar gum, hair conditioner (18n195-1m), hexylene glycol, high density polyethylene, hyaluronate sodium, hydrocarbon gel, plasticized, hydrochloric acid, hydrochloric acid, diluted, hydrogen peroxide, hydrogenated castor oil, hydrogenated palm/palm kernel oil peg-6 esters, hydroxyethyl cellulose, hydroxymethyl cellulose, hydroxyoctacosanyl hydroxystearate, hydroxypropyl cellulose, hypromelloses, imidurea, irish moss extract, isobutane, isoceteth-20, isooctyl acrylate, isopropyl alcohol, isopropyl isostearate, isopropyl myristate, isopropyl myristate-myristyl alcohol, isopropyl palmitate, isopropyl stearate, isostearic acid, isostearyl alcohol, jelene, kaolin, kathon cg, kathon cg ii, lactate, lactic acid, lactic acid, dl-, laneth, lanolin, lanolin alcohol-mineral oil, lanolin alcohols, lanolin anhydrous, lanolin cholesterols, lanolin, ethoxylated, lanolin, hydrogenated, lauramine oxide, laurdimonium hydrolyzed animal collagen, laureth sulfate, laureth-2, laureth-23, laureth-4, lauric diethanolamide, lauric myristic diethanolamide, lauryl sulfate, lavandula angustifolia flowering top, lecithin, lecithin unbleached, lemon oil, light mineral oil, light mineral oil (85 ssu), limonene, (+/−)-, lipocol sc-15, magnesium aluminum silicate, magnesium aluminum silicate hydrate, magnesium nitrate, magnesium stearate, mannitol, maprofix, medical antiform a-f emulsion, menthol, methyl gluceth-10, methyl gluceth-20, methyl gluceth-20 sesquistearate, methyl glucose sesquistearate, methyl salicylate, methyl stearate, methylcelluloses, methylchloroisothiazolinone, methylisothiazolinone, methylparaben, microcrystalline wax, mineral oil, mono and diglyceride, monostearyl citrate, multisterol extract, myristyl alcohol, myristyl lactate, niacinamide, nitric acid, nitrogen, nonoxynol iodine, nonoxynol-15, nonoxynol-9, oatmeal, octadecene-1/maleic acid copolymer, octoxynol-1, octoxynol-9, octyldodecanol, oleic acid, oleth-10/oleth-5, oleth-2, oleth-20, oleyl alcohol, oleyl oleate, olive oil, palmitamine oxide, parabens, paraffin, paraffin, white soft, parfum creme 45/3, peanut oil, peanut oil, refined, pectin, peg 6-32 stearate/glycol stearate, peg-100 stearate, peg-12 glyceryl laurate, peg-120 glyceryl stearate, peg-120 methyl glucose dioleate, peg-15 cocamine, peg-150 distearate, peg-2 stearate, peg-22 methyl ether/dodecyl glycol copolymer, peg-25 propylene glycol stearate, peg-4 dilaurate, peg-4 laurate, peg-45/dodecyl glycol copolymer, peg-5 oleate, peg-50 stearate, peg-54 hydrogenated castor oil, peg-6 isostearate, peg-60 hydrogenated castor oil, peg-7 methyl ether, peg-75 lanolin, peg-8 laurate, peg-8 stearate, pegoxol 7 stearate, pentaerythritol cocoate, peppermint oil, perfume 25677, perfume bouquet, perfume e-1991, perfume gd 5604, perfume tana 90/42 scba, perfume w-1952-1, petrolatum, petrolatum, white, petroleum distillates, phenonip, phenoxyethanol, phenylmercuric acetate, phosphoric acid, pine needle oil (pinus sylvestris), plastibase-50w, polidronium chloride, poloxamer 124, poloxamer 181, poloxamer 182, poloxamer 188, poloxamer 237, poloxamer 407, polycarbophil, polyethylene glycol 1000, polyethylene glycol 1450, polyethylene glycol 1500, polyethylene glycol 1540, polyethylene glycol 200, polyethylene glycol 300, polyethylene glycol 300-1600, polyethylene glycol 3350, polyethylene glycol 400, polyethylene glycol 4000, polyethylene glycol 540, polyethylene glycol 600, polyethylene glycol 6000, polyethylene glycol 8000, polyethylene glycol 900, polyhydroxyethyl methacrylate, polyisobutylene, polyisobutylene (1100000 mw), polyoxyethylene-polyoxypropylene 1800, polyoxyethylene alcohols, polyoxyethylene fatty acid esters, polyoxyethylene propylene, polyoxyl 20 cetostearyl ether, polyoxyl 40 hydrogenated castor oil, polyoxyl 40 stearate, polyoxyl 400 stearate, polyoxyl 6 and polyoxyl 32 palmitostearate, polyoxyl distearate, polyoxyl glyceryl stearate, polyoxyl lanolin, polyoxyl stearate, polypropylene, polyquaternium-10, polysorbate 20, polysorbate 40, polysorbate 60, polysorbate 65, polysorbate 80, polyvinyl alcohol, potash, potassium citrate, potassium hydroxide, potassium soap, potassium sorbate, povidone acrylate copolymer, povidone hydrogel, povidone k90, povidone/eicosene copolymer, povidones, ppg-12/smdi copolymer, ppg-15 stearyl ether, ppg-20 methyl glucose ether distearate, ppg-26 oleate, product wat, promulgen d, promulgen g, propane, propellant a-46, propyl gallate, propylene carbonate, propylene glycol, propylene glycol diacetate, propylene glycol dicaprylate, propylene glycol monopalmitostearate, propylene glycol palmitostearate, propylene glycol ricinoleate, propylene glycol/diazolidinyl urea/methylparaben/propylparben, propylparaben, protein hydrolysate, quaternium-15, quaternium-15 cis-form, quaternium-52, saccharin, saccharin sodium, safflower oil, sd alcohol 3a, sd alcohol 40, sd alcohol 40-2, sd alcohol 40b, sepineo p 600, shea butter, silicon, silicon dioxide, silicone, silicone adhesive bio-psa q7-4201, silicone adhesive bio-psa q7-4301, silicone emulsion, simethicone, simethicone emulsion, sipon is 20np, sodium acetate, sodium acetate anhydrous, sodium alkyl sulfate, sodium benzoate, sodium bisulfite, sodium borate, sodium cetostearyl sulfate, sodium chloride, sodium citrate, sodium cocoyl sarcosinate, sodium dodecylbenzenesulfonate, sodium formaldehyde sulfoxylate, sodium hydroxide, sodium iodide, sodium lactate, sodium laureth-2 sulfate, sodium laureth-3 sulfate, sodium laureth-5 sulfate, sodium lauroyl sarcosinate, sodium lauryl sulfate, sodium lauryl sulfoacetate, sodium metabisulfite, sodium phosphate, sodium phosphate, dibasic, sodium phosphate, dibasic, anhydrous, sodium phosphate, dibasic, dihydrate, sodium phosphate, dibasic, heptahydrate, sodium phosphate, monobasic, sodium phosphate, monobasic, anhydrous, sodium phosphate, monobasic, dihydrate, sodium phosphate, monobasic, monohydrate, sodium polyacrylate (2500000 mw), sodium pyrrolidone carboxylate, sodium sulfite, sodium sulfosuccinated undecyclenic monoalkylolamide, sodium thiosulfate, sodium xylenesulfonate, somay 44, sorbic acid, sorbitan, sorbitan isostearate, sorbitan monolaurate, sorbitan monooleate, sorbitan monopalmitate, sorbitan monostearate, sorbitan sesquioleate, sorbitan tristearate, sorbitol, sorbitol solution, soybean flour, soybean oil, spearmint oil, spermaceti, squalane, starch, stearalkonium chloride, stearamidoethyl diethylamine, steareth-10, steareth-100, steareth-2, steareth-20, steareth-21, steareth-40, stearic acid, stearic diethanolamide, stearoxytrimethylsilane, steartrimonium hydrolyzed animal collagen, stearyl alcohol, styrene/isoprene/styrene block copolymer, sucrose, sucrose distearate, sucrose polyesters, sulfacetamide sodium, sulfuric acid, surfactol qs, talc, tall oil, tallow glycerides, tartaric acid, tenox, tenox-2, tert-butyl alcohol, tert-butyl hydroperoxide, thimerosal, titanium dioxide, tocopherol, tocophersolan, trichloromonofluoromethane, trideceth-10, triethanolamine lauryl sulfate, triglycerides, medium chain, trihydroxystearin, trilaneth-4 phosphate, trilaureth-4 phosphate, trisodium citrate dihydrate, trisodium hedta, triton x-200, trolamine, tromethamine, tyloxapol, undecylenic acid, vegetable oil, vegetable oil, hydrogenated, viscarin, vitamin E, wax, emulsifying, wecobee fs, white wax, xanthan gum and zinc acetate.

A pharmaceutical antibody composition for transdermal administration may comprise at least one inactive ingredient. Any or none of the inactive ingredients used may have been approved by the US Food and Drug Administration (FDA). A non-exhaustive list of inactive ingredients for use in pharmaceutical compositions for transdermal administration includes acrylates copolymer, acrylic acid-isooctyl acrylate copolymer, acrylic adhesive 788, adcote 72a103, aerotex resin 3730, alcohol, alcohol, dehydrated, aluminum polyester, bentonite, butylated hydroxytoluene, butylene glycol, butyric acid, caprylic/capric triglyceride, carbomer 1342, carbomer 940, carbomer 980, carrageenan, cetylpyridinium chloride, citric acid, crospovidone, daubert 1-5 pestr (matte) 164z, diethylene glycol monoethyl ether, diethylhexyl phthalate, dimethicone copolyol, dimethicone mdx4-4210, dimethicone medical fluid 360, dimethylaminoethyl methacrylate-butyl methacrylate-methyl methacrylate copolymer, dipropylene glycol, duro-tak 280-2516, duro-tak 387-2516, duro-tak 80-1196, duro-tak 87-2070, duro-tak 87-2194, duro-tak 87-2287, duro-tak 87-2296, duro-tak 87-2888, duro-tak 87-2979, edetate disodium, ethyl acetate, ethyl oleate, ethylcelluloses, ethylene vinyl acetate copolymer, ethylene-propylene copolymer, fatty acid esters, gelva 737, glycerin, glyceryl laurate, glyceryl oleate, heptane, high density polyethylene, hydrochloric acid, hydrogenated polybutene 635-690, hydroxyethyl cellulose, hydroxypropyl cellulose, isopropyl myristate, isopropyl palmitate, lactose, lanolin anhydrous, lauryl lactate, lecithin, levulinic acid, light mineral oil, medical adhesive modified s-15, methyl alcohol, methyl laurate, mineral oil, nitrogen, octisalate, octyldodecanol, oleic acid, oleyl alcohol, oleyl oleate, pentadecalactone, petrolatum, white, polacrilin, polyacrylic acid (250000 mw), polybutene (1400 mw), polyester, polyester polyamine copolymer, polyester rayon, polyethylene terephthalates, polyisobutylene, polyisobutylene (1100000 mw), polyisobutylene (35000 mw), polyisobutylene 178-236, polyisobutylene 241-294, polyisobutylene 35-39, polyisobutylene low molecular weight, polyisobutylene medium molecular weight, polyisobutylene/polybutene adhesive, polypropylene, polyvinyl acetate, polyvinyl alcohol, polyvinyl chloride, polyvinyl chloride-polyvinyl acetate copolymer, polyvinylpyridine, povidone k29/32, povidones, propylene glycol, propylene glycol monolaurate, ra-2397, ra-3011, silicon, silicon dioxide, colloidal, silicone, silicone adhesive 4102, silicone adhesive 4502, silicone adhesive bio-psa q7-4201, silicone adhesive bio-psa q7-4301, silicone/polyester film strip, sodium chloride, sodium citrate, sodium hydroxide, sorbitan monooleate, stearalkonium hectorite/propylene carbonate, titanium dioxide, triacetin, trolamine, tromethamine, union 76 amsco-res 6038 and viscose/cotton.

A pharmaceutical antibody composition for intradermal administration may comprise at least one inactive ingredient. Any or none of the inactive ingredients used may have been approved by the US Food and Drug Administration (FDA). A non-exhaustive list of inactive ingredients for use in pharmaceutical compositions for intradermal administration includes benzalkonium chloride, benzyl alcohol, carboxymethylcellulose sodium, creatinine, edetate disodium, glycerin, hydrochloric acid, metacresol, methylparaben, phenol, polysorbate 80, protamine sulfate, sodium acetate, sodium bisulfate, sodium chloride, sodium hydroxide, sodium phosphate, sodium phosphate, dibasic, sodium phosphate, dibasic, heptahydrate, sodium phosphate, monobasic, anhydrous and zinc chloride.

### Depot Administration

As described herein, in some embodiments, the composition is formulated in depots for extended release. Generally, a specific organ or tissue (a “target tissue”) is targeted for administration.

In some aspects of the invention, the antibody compositions are spatially retained within or proximal to a target tissue. Provided are method of providing a composition to a target tissue of a mammalian subject by contacting the target tissue (which contains one or more target cells) with the composition under conditions such that the composition, in particular the nucleic acid component(s) of the composition, is substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue. Advantageously, retention is determined by measuring the amount of the nucleic acid present in the composition that enters one or more target cells. For example, at least 1, 5, 10, 20, 30, 40, 50, 60, 70, 80, 85, 90, 95, 96, 97, 98, 99, 99.9, 99.99 or greater than 99.99% of the nucleic acids administered to the subject are present intracellularly at a period of time following administration. For example, intramuscular injection to a mammalian subject is performed using an aqueous composition containing a ribonucleic acid and a transfection reagent, and retention of the composition is determined by measuring the amount of the ribonucleic acid present in the muscle cells.

Aspects of the invention are directed to methods of providing a composition to a target tissue of a mammalian subject, by contacting the target tissue (containing one or more target cells) with the composition under conditions such that the composition is substantially retained in the target tissue. The composition contains an effective amount of polynucleotides such that the polypeptide of interest is produced in at least one target cell. The compositions generally contain a cell penetration agent, although “naked” antibody composition (such as nucleic acids without a cell penetration agent or other agent) is also contemplated, and a pharmaceutically acceptable carrier.

In some circumstances, the amount of a protein produced by cells in a tissue is desirably increased. Preferably, this increase in protein production is spatially restricted to cells within the target tissue. Thus, provided are methods of increasing production of a protein of interest in a tissue of a mammalian subject. A composition is provided that contains polynucleotides characterized in that a unit quantity of composition has been determined to produce the polypeptide of interest in a substantial percentage of cells contained within a predetermined volume of the target tissue.

In some embodiments, the antibody composition includes a plurality of different polynucleotides, where one or more than one of the polynucleotides encodes a polypeptide of interest. Optionally, the composition also contains a cell penetration agent to assist in the intracellular delivery of the composition. A determination is made of the dose of the composition required to produce the polypeptide of interest in a substantial percentage of cells contained within the predetermined volume of the target tissue (generally, without inducing significant production of the polypeptide of interest in tissue adjacent to the predetermined volume, or distally to the target tissue). Subsequent to this determination, the determined dose is introduced directly into the tissue of the mammalian subject.

In one embodiment, the invention provides for the antibody compositions to be delivered in more than one injection or by split dose injections.

In one embodiment, the invention may be retained near target tissue using a small disposable drug reservoir, patch pump or osmotic pump. Non-limiting examples of patch pumps include those manufactured and/or sold by BD® (Franklin Lakes, N.J.), Insulet Corporation (Bedford, Mass.), SteadyMed Therapeutics (San Francisco, Calif.), Medtronic (Minneapolis, Minn.) (e.g., MiniMed), UniLife (York, Pa.), Valeritas (Bridgewater, N.J.), and SpringLeaf Therapeutics (Boston, Mass.). A non-limiting example of an osmotic pump include those manufactured by DURECT® (Cupertino, Calif.) (e.g., DUROS® and ALZET®).

### Pulmonary Administration

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

As a non-limiting example, the antibody compositions described herein may be formulated for pulmonary delivery by the methods described in U.S. Pat. No. 8,257,685; herein incorporated by reference in its entirety.

Pharmaceutical antibody compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

The compositions and formulations provided herein which may be used for pulmonary delivery may further comprise one or more surfactants. Suitable surfactants or surfactant components for enhancing the uptake of the compositions of the invention include synthetic and natural as well as full and truncated forms of surfactant protein A, surfactant protein B, surfactant protein C, surfactant protein D and surfactant Protein E, di-saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine; phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; as well as natural and artificial lamellar bodies which are the natural carrier vehicles for the components of surfactant, omega-3 fatty acids, polyenic acid, polyenoic acid, lecithin, palmitinic acid, non-ionic block copolymers of ethylene or propylene oxides, polyoxypropylene, monomeric and polymeric, polyoxyethylene, monomeric and polymeric, poly(vinyl amine) with dextran and/or alkanoyl side chains, Brij 35, Triton X-100 and synthetic surfactants ALEC, Exosurf, Survan and Atovaquone, among others. These surfactants can be used either as single or part of a multiple component surfactant in a formulation, or as covalently bound additions to the 5′ and/or 3′ ends of the nucleic acid component of a pharmaceutical composition herein.

### Intranasal, Nasal and Buccal Administration

Formulations described herein as being useful for pulmonary delivery are useful for intranasal delivery of an antibody pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical antibody composition for inhalation (respiratory) administration may comprise at least one inactive ingredient. Any or none of the inactive ingredients used may have been approved by the US Food and Drug Administration (FDA). A non-exhaustive list of inactive ingredients for use in pharmaceutical compositions for inhalation (respiratory) administration includes acetone sodium bisulfite, acetylcysteine, alcohol, alcohol, dehydrated, ammonia, apaflurane, ascorbic acid, benzalkonium chloride, calcium carbonate, carbon dioxide, cetylpyridinium chloride, chlorobutanol, citric acid, d&c yellow no. 10, dichlorodifluoromethane, dichlorotetrafluoroethane, edetate disodium, edetate sodium, fd&c yellow no. 6, fluorochlorohydrocarbons, gelatin, glycerin, glycine, hydrochloric acid, hydrochloric acid, diluted, lactose, lactose monohydrate, lecithin, lecithin, hydrogenated soy, lecithin, soybean, lysine monohydrate, mannitol, menthol, methylparaben, nitric acid, nitrogen, norflurane, oleic acid, polyethylene glycol 1000, povidone k25, propylene glycol, propylparaben, saccharin, saccharin sodium, silicon dioxide, colloidal, sodium bisulfate, sodium bisulfite, sodium chloride, sodium citrate, sodium hydroxide, sodium lauryl sulfate, sodium metabisulfite, sodium sulfate anhydrous, sodium sulfite, sorbitan trioleate, sulfuric acid, thymol, titanium dioxide, trichloromonofluoromethane, tromethamine and zinc oxide.

A pharmaceutical antibody composition for nasal administration may comprise at least one inactive ingredient. Any or none of the inactive ingredients used may have been approved by the US Food and Drug Administration (FDA). A non-exhaustive list of inactive ingredients for use in pharmaceutical compositions for nasal administration includes acetic acid, alcohol, dehydrated, allyl .alpha.-ionone, anhydrous dextrose, anhydrous trisodium citrate, benzalkonium chloride, benzethonium chloride, benzyl alcohol, butylated hydroxyanisole, butylated hydroxytoluene, caffeine, carbon dioxide, carboxymethylcellulose sodium, cellulose, microcrystalline, chlorobutanol, citric acid, citric acid monohydrate, dextrose, dichlorodifluoromethane, dichlorotetrafluoroethane, edetate disodium, glycerin, glycerol ester of hydrogenated rosin, hydrochloric acid, hypromellose 2910 (15000 mpa·s), methylcelluloses, methylparaben, nitrogen, norflurane, oleic acid, petrolatum, white, phenylethyl alcohol, polyethylene glycol 3350, polyethylene glycol 400, polyoxyl 400 stearate, polysorbate 20, polysorbate 80, potassium phosphate, monobasic, potassium sorbate, propylene glycol, propylparaben, sodium acetate, sodium chloride, sodium citrate, sodium hydroxide, sodium phosphate, sodium phosphate, dibasic, sodium phosphate, dibasic, anhydrous, sodium phosphate, dibasic, dihydrate, sodium phosphate, dibasic, dodecahydrate, sodium phosphate, dibasic, heptahydrate, sodium phosphate, monobasic, anhydrous, sodium phosphate, monobasic, dihydrate, sorbitan trioleate, sorbitol, sorbitol solution, sucralose, sulfuric acid, trichloromonofluoromethane and trisodium citrate dihydrate.

### Ophthalmic and Auricular (Otic) Administration

A pharmaceutical antibody composition may be prepared, packaged, and/or sold in a formulation suitable for delivery to and/or around the eye and/or delivery to the ear (e.g., auricular (otic) administration). Non-limiting examples of route of administration for delivery to and/or around the eye include retrobulbar, conjuctival, intracorneal, intraocular, intravitreal, ophthlamic and subconjuctiva. Such formulations may, for example, be in the form of eye drops or ear drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this invention. A multilayer thin film device may be prepared to contain a pharmaceutical composition for delivery to the eye and/or surrounding tissue.

A pharmaceutical antibody composition for ophthalmic administration may comprise at least one inactive ingredient. Any or none of the inactive ingredients used may have been approved by the US Food and Drug Administration (FDA). A non-exhaustive list of inactive ingredients for use in pharmaceutical compositions for ophthalmic administration includes acetic acid, alcohol, alcohol, dehydrated, alginic acid, amerchol-cab, ammonium hydroxide, anhydrous trisodium citrate, antipyrine, benzalkonium chloride, benzethonium chloride, benzododecinium bromide, boric acid, caffeine, calcium chloride, carbomer 1342, carbomer 934p, carbomer 940, carbomer homopolymer type b (allyl pentaerythritol crosslinked), carboxymethylcellulose sodium, castor oil, cetyl alcohol, chlorobutanol, chlorobutanol, anhydrous, cholesterol, citric acid, citric acid monohydrate, creatinine, diethanolamine, diethylhexyl phthalate, divinylbenzene styrene copolymer, edetate disodium, edetate disodium anhydrous, edetate sodium, ethylene vinyl acetate copolymer, gellan gum (low acyl), glycerin, glyceryl stearate, high density polyethylene, hydrocarbon gel, plasticized, hydrochloric acid, hydrochloric acid, diluted, hydroxyethyl cellulose, hydroxypropyl methylcellulose 2906, hypromellose 2910 (15000 mpa·s), hypromelloses, jelene, lanolin, lanolin alcohols, lanolin anhydrous, lanolin nonionic derivatives, lauralkonium chloride, lauroyl sarcosine, light mineral oil, magnesium chloride, mannitol, methylcellulose (4000 mpa·s), methylcelluloses, methylparaben, mineral oil, nitric acid, nitrogen, nonoxynol-9, octoxynol-40, octylphenol polymethylene, petrolatum, petrolatum, white, phenylethyl alcohol, phenylmercuric acetate, phenylmercuric nitrate, phosphoric acid, polidronium chloride, poloxamer 188, poloxamer 407, polycarbophil, polyethylene glycol 300, polyethylene glycol 400, polyethylene glycol 8000, polyoxyethylene-polyoxypropylene 1800, polyoxyl 35 castor oil, polyoxyl 40 hydrogenated castor oil, polyoxyl 40 stearate, polypropylene glycol, polysorbate 20, polysorbate 60, polysorbate 80, polyvinyl alcohol, potassium acetate, potassium chloride, potassium phosphate, monobasic, potassium sorbate, povidone k29/32, povidone k30, povidone k90, povidones, propylene glycol, propylparaben, soda ash, sodium acetate, sodium bisulfate, sodium bisulfite, sodium borate, sodium borate decahydrate, sodium carbonate, sodium carbonate monohydrate, sodium chloride, sodium citrate, sodium hydroxide, sodium metabisulfite, sodium nitrate, sodium phosphate, sodium phosphate dihydrate, sodium phosphate, dibasic, sodium phosphate, dibasic, anhydrous, sodium phosphate, dibasic, dihydrate, sodium phosphate, dibasic, heptahydrate, sodium phosphate, monobasic, sodium phosphate, monobasic, anhydrous, sodium phosphate, monobasic, dihydrate, sodium phosphate, monobasic, monohydrate, sodium sulfate, sodium sulfate anhydrous, sodium sulfate decahydrate, sodium sulfite, sodium thiosulfate, sorbic acid, sorbitan monolaurate, sorbitol, sorbitol solution, stabilized oxychloro complex, sulfuric acid, thimerosal, titanium dioxide, tocophersolan, trisodium citrate dihydrate, triton 720, tromethamine, tyloxapol and zinc chloride.

A pharmaceutical antibody composition for retrobulbar administration may comprise at least one inactive ingredient. Any or none of the inactive ingredients used may have been approved by the US Food and Drug Administration (FDA). A non-exhaustive list of inactive ingredients for use in pharmaceutical compositions for retrobulbar administration includes hydrochloric acid and sodium hydroxide.

A pharmaceutical antibody composition for intraocular administration may comprise at least one inactive ingredient. Any or none of the inactive ingredients used may have been approved by the US Food and Drug Administration (FDA). A non-exhaustive list of inactive ingredients for use in pharmaceutical compositions for intraocular administration includes benzalkonium chloride, calcium chloride, citric acid monohydrate, hydrochloric acid, magnesium chloride, polyvinyl alcohol, potassium chloride, sodium acetate, sodium chloride, sodium citrate and sodium hydroxide.

A pharmaceutical antibody composition for intravitreal administration may comprise at least one inactive ingredient. Any or none of the inactive ingredients used may have been approved by the US Food and Drug Administration (FDA). A non-exhaustive list of inactive ingredients for use in pharmaceutical compositions for intravitreal administration includes calcium chloride, carboxymethylcellulose sodium, cellulose, microcrystalline, hyaluronate sodium, hydrochloric acid, magnesium chloride, magnesium stearate, polysorbate 80, polyvinyl alcohol, potassium chloride, sodium acetate, sodium bicarbonate, sodium carbonate, sodium chloride, sodium hydroxide, sodium phosphate dibasic heptahydrate, sodium phosphate monobasic monohydrate and trisodium citrate dehydrate.

A pharmaceutical antibody composition for subconjunctival administration may comprise at least one inactive ingredient. Any or none of the inactive ingredients used may have been approved by the US Food and Drug Administration (FDA). A non-exhaustive list of inactive ingredients for use in pharmaceutical compositions for subconjunctival administration includes benzyl alcohol, hydrochloric acid and sodium hydroxide.

A pharmaceutical antibody composition for auricular administration may comprise at least one inactive ingredient. Any or none of the inactive ingredients used may have been approved by the US Food and Drug Administration (FDA). A non-exhaustive list of inactive ingredients for use in pharmaceutical compositions for auricular administration includes acetic acid, aluminum acetate, aluminum sulfate anhydrous, benzalkonium chloride, benzethonium chloride, benzyl alcohol, boric acid, calcium carbonate, cetyl alcohol, chlorobutanol, chloroxylenol, citric acid, creatinine, cupric sulfate, cupric sulfate anhydrous, edetate disodium, edetic acid, glycerin, glyceryl stearate, hydrochloric acid, hydrocortisone, hydroxyethyl cellulose, isopropyl myristate, lactic acid, lecithin, hydrogenated, methylparaben, mineral oil, petrolatum, petrolatum, white, phenylethyl alcohol, polyoxyl 40 stearate, polyoxyl stearate, polysorbate 20, polysorbate 80, polyvinyl alcohol, potassium metabisulfite, potassium phosphate, monobasic, povidone k90f, povidones, propylene glycol, propylene glycol diacetate, propylparaben, sodium acetate, sodium bisulfite, sodium borate, sodium chloride, sodium citrate, sodium hydroxide, sodium phosphate, dibasic, anhydrous, sodium phosphate, dibasic, heptahydrate, sodium phosphate, monobasic, anhydrous, sodium sulfite, sulfuric acid and thimerosal.

### Payload Administration: Detectable Agents and Therapeutic Agents

The antibody compositions described herein can be used in a number of different scenarios in which delivery of a substance (the “payload”) to a biological target is desired, for example delivery of detectable substances for detection of the target, or delivery of a therapeutic agent. Detection methods can include, but are not limited to, both imaging in vitro and in vivo imaging methods, e.g., immunohistochemistry, bioluminescence imaging (BLI), Magnetic Resonance Imaging (MM), positron emission tomography (PET), electron microscopy, X-ray computed tomography, Raman imaging, optical coherence tomography, absorption imaging, thermal imaging, fluorescence reflectance imaging, fluorescence microscopy, fluorescence molecular tomographic imaging, nuclear magnetic resonance imaging, X-ray imaging, ultrasound imaging, photoacoustic imaging, lab assays, or in any situation where tagging/staining/imaging is required.

Antibody compositions described herein can be used in intracellular targeting of a payload, e.g., detectable or therapeutic agent, to specific organelle. Exemplary intracellular targets can include, but are not limited to, the nuclear localization for advanced mRNA processing, or a nuclear localization sequence (NLS) linked to the mRNA containing an inhibitor.

In addition, the antibody compositions described herein can be used to deliver therapeutic agents to cells or tissues, e.g., in living animals. For example, the antibody compositions described herein can be used to deliver highly polar chemotherapeutics agents to kill cancer cells. The antibody compositions attached to the therapeutic agent through a linker can facilitate member permeation allowing the therapeutic agent to travel into a cell to reach an intracellular target.

In some embodiments, the payload may be a therapeutic agent such as a cytotoxin, radioactive ion, chemotherapeutic, or other therapeutic agent. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020 incorporated herein in its entirety), rachelmycin (CC-1065, see U.S. Pat. Nos. 5,475,092, 5,585,499, and 5,846,545, all of which are incorporated herein by reference), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium. Other therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In some embodiments, the payload may be a detectable agent, such as various organic small molecules, inorganic compounds, nanoparticles, enzymes or enzyme substrates, fluorescent materials, luminescent materials (e.g., luminol), bioluminescent materials (e.g., luciferase, luciferin, and aequorin), chemiluminescent materials, radioactive materials (e.g., ¹⁸F, ⁶⁷Ga, ^{81m}Kr, ⁸²Rb, ¹¹¹In, ¹²³I, ¹³³Xe, ²⁰¹Tl, ¹²⁵I, ³⁵S, ¹⁴C, ³H, or ^{99m}Tc (e.g., as pertechnetate (technetate(VII), TcO₄⁻)), and contrast agents (e.g., gold (e.g., gold nanoparticles), gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons). Such optically-detectable labels include for example, without limitation, 4-acetamido-4′-isothiocyanatostilbene-2,2′disulfonic acid; acridine and derivatives (e.g., acridine and acridine isothiocyanate); 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives (e.g., coumarin, 7-amino-4-methylcoumarin (AMC, Coumarin 120), and 7-amino-4-trifluoromethylcoumarin (Coumarin 151)); cyanine dyes; cyanosine; 4′,6-diaminidino-2-phenylindole (DAPI); 5′ 5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]-naphthalene-1-sulfonyl chloride (DNS, dansylchloride); 4-dimethylaminophenylazophenyl-4′-isothiocyanate (DABITC); eosin and derivatives (e.g., eosin and eosin isothiocyanate); erythrosin and derivatives (e.g., erythrosin B and erythrosin isothiocyanate); ethidium; fluorescein and derivatives (e.g., 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, X-rhodamine-5-(and -6)-isothiocyanate (QFITC or XRITC), and fluorescamine); 2-[2-[3-[[1,3-dihydro-1,1-dimethyl-3-(3-sulfopropyl)-2H-benz[e]indol-2-ylidene]ethylidene]-2-[4-(ethoxycarbonyl)-1-piperazinyl]-1-cyclopenten-1-yl]ethenyl]-1,1-dimethyl-3-(3-sulforpropyl)-1H-benz[e]indolium hydroxide, inner salt, compound with n,n-diethylethanamine (1:1) (IR144); 5-chloro-2-[2-[3-[(5-chloro-3-ethyl-2(3H)-benzothiazol-ylidene)ethylidene]-2-(diphenylamino)-1-cyclopenten-1-yf]ethenyl]-3-ethyl benzothiazolium perchlorate (IR140); Malachite Green isothiocyanate; 4-methylumbelliferone orthocresolphthalein; nitrotyrosine; pararosaniline; Phenol Red; B-phycoerythrin; o-phthaldialdehyde; pyrene and derivatives (e.g., pyrene, pyrene butyrate, and succinimidyl 1-pyrene); butyrate quantum dots; Reactive Red 4 (CIBACRON™ Brilliant Red 3B-A); rhodamine and derivatives (e.g., 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′tetramethyl-6-carboxyrhodamine (TAMRA) tetramethyl rhodamine, and tetramethyl rhodamine isothiocyanate (TRITC)); riboflavin; rosolic acid; terbium chelate derivatives; Cyanine-3 (Cy3); Cyanine-5 (Cy5); cyanine-5.5 (Cy5.5), Cyanine-7 (Cy7); IRD 700; IRD 800; Alexa 647; La Jolta Blue; phthalo cyanine; and naphthalo cyanine.

In some embodiments, the detectable agent may be a non-detectable precursor that becomes detectable upon activation (e.g., fluorogenic tetrazine-fluorophore constructs (e.g., tetrazine-BODIPY FL, tetrazine-Oregon Green 488, or tetrazine-BODIPY TMR-X) or enzyme activatable fluorogenic agents (e.g., PROSENSE® (VisEn Medical))). In vitro assays in which the enzyme labeled compositions can be used include, but are not limited to, enzyme linked immunosorbent assays (ELISAs), immunoprecipitation assays, immunofluorescence, enzyme immunoassays (EIA), radioimmunoassays (RIA), and Western blot analysis.

### Combinations

The antibody polynucleotides or compositions may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

Such combinations may include any of the agents identified in copending International application number PCT/US2014/069155 (Attorney Docket Number M073), the contents of which are incorporated herein by reference in their entirety.

The combinations referred to above can conveniently be presented for use in the form of a pharmaceutical formulation and thus pharmaceutical compositions comprising a combination as defined above together with a pharmaceutically acceptable diluent or carrier represent a further aspect of the invention.

The individual compounds of such combinations can be administered either sequentially or simultaneously in separate or combined pharmaceutical formulations. In one embodiment, the individual compounds will be administered simultaneously in a combined pharmaceutical formulation.

It will further be appreciated that therapeutically, prophylactically, diagnostically, or imaging active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually. In one embodiment, the combinations, each or together may be administered according to the split dosing regimens described herein.

### Dosing

The present invention provides methods comprising administering antibody compositions and in accordance with the invention to a subject in need thereof. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

According to the present invention, antibody compositions may be administered in split-dose regimens. As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the antibody compositions of the present invention are administered to a subject in split doses. The antibody compositions may be formulated in buffer only or in a formulation described herein.

### Dosage Forms

Antibody pharmaceutical compositions described herein can be formulated into a dosage form described herein, such as a topical, intranasal, intratracheal, or injectable (e.g., intravenous, intraocular, intravitreal, intramuscular, intracardiac, intraperitoneal, subcutaneous).

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

### Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption then depends upon its rate of dissolution which, in turn, may depend upon crystal size and crystalline form. Alternatively, delayed absorption of a parenterally administered antibody composition may be accomplished by dissolving or suspending the antibody polynucleotide or composition in an oil vehicle. Injectable depot forms are made by forming microencapsule matrices of the composition in biodegradable polymers such as polylactide-polyglycolide. Depending upon the ratio of polynucleotide to polymer and the nature of the particular polymer employed, the rate of polynucleotides release can be controlled. Examples of other biodegradable polymers include, but are not limited to, poly(orthoesters) and poly(anhydrides). Depot injectable formulations may be prepared by entrapping the polynucleotides in liposomes or microemulsions which are compatible with body tissues.

### Pulmonary

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 μm to 500 μm. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21^{st} ed., Lippincott Williams & Wilkins, 2005 (the contents of which are incorporated herein by reference in their entirety).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Multi-Dose and Repeat-Dose Administration

In some embodiments, antibody compounds and/or compositions of the present invention may be administered in two or more doses (referred to herein as “multi-dose administration”). Such doses may comprise the same components or may comprise components not included in a previous dose. Such doses may comprise the same mass and/or volume of components or an altered mass and/or volume of components in comparison to a previous dose. In some embodiments, multi-dose administration may comprise repeat-dose administration. As used herein, the term “repeat-dose administration” refers to two or more doses administered consecutively or within a regimen of repeat doses comprising substantially the same components provided at substantially the same mass and/or volume. In some embodiments, subjects may display a repeat-dose response.

As used herein, the term “repeat-dose response” refers to a response in a subject to a repeat-dose that differs from that of another dose administered within a repeat-dose administration regimen. In some embodiments, such a response may be the expression of a protein in response to a repeat-dose comprising a polynucleotide. In such embodiments, protein expression may be elevated in comparison to another dose administered within a repeat-dose administration regimen or protein expression may be reduced in comparison to another dose administered within a repeat-dose administration regimen. Alteration of protein expression may be from about 1% to about 20%, from about 5% to about 50% from about 10% to about 60%, from about 25% to about 75%, from about 40% to about 100% and/or at least 100%. A reduction in expression of mRNA administered as part of a repeat-dose regimen, wherein the level of protein translated from the administered RNA is reduced by more than 40% in comparison to another dose within the repeat-dose regimen is referred to herein as “repeat-dose resistance.”

### Properties of the Pharmaceutical Compositions

The pharmaceutical compositions described herein can be characterized by one or more of the following properties:

### Bioavailability

The polynucleotides or compositions when formulated into a composition with a delivery agent as described herein, can exhibit an increase in bioavailability as compared to a composition lacking a delivery agent as described herein. As used herein, the term “bioavailability” refers to the systemic availability of a given amount of polynucleotides or compositions administered to a mammal. Bioavailability can be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, herein incorporated by reference in its entirety.

The Cₘₐₓ value is the maximum concentration of the compound achieved in the serum or plasma of a mammal following administration of the compound to the mammal. The Cₘₐₓ value of a particular compound can be measured using methods known to those of ordinary skill in the art. The phrases “increasing bioavailability” or “improving the pharmacokinetics,” as used herein mean that the systemic availability of a first polynucleotide or composition, measured as AUC, Cₘₐₓ, or Cₘᵢₙ in a mammal is greater, when co-administered with a delivery agent as described herein, than when such co-administration does not take place. In some embodiments, the bioavailability of the polynucleotide or composition can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

In some embodiments, liquid formulations of the polynucleotide or composition may have varying in vivo half-life, requiring modulation of doses to yield a therapeutic effect. To address this, in some embodiments of the present invention, the polynucleotide or composition formulations may be designed to improve bioavailability and/or therapeutic effect during repeat administrations. Such formulations may enable sustained release or degradation rates by nucleases.

In some embodiments, cationic nanoparticles comprising combinations of divalent and monovalent cations may be formulated with the polynucleotide or composition. Such nanoparticles may form spontaneously in solution over a given period (e.g. hours, days, etc). Such nanoparticles do not form in the presence of divalent cations alone or in the presence of monovalent cations alone. The delivery of the polynucleotide or composition in cationic nanoparticles or in one or more depot comprising cationic nanoparticles may improve antibody bioavailability by acting as a long-acting depot and/or reducing the rate of degradation by nucleases.

### Therapeutic Window

The polynucleotide or composition, when formulated into a composition with a delivery agent as described herein, can exhibit an increase in the therapeutic window of the administered polynucleotide or composition as compared to the therapeutic window of the administered polynucleotide or composition lacking a delivery agent as described herein. As used herein “therapeutic window” refers to the range of plasma concentrations, or the range of levels of therapeutically active substance at the site of action, with a high probability of eliciting a therapeutic effect. In some embodiments, the therapeutic window of the polynucleotide or composition when co-administered with a delivery agent as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

The polynucleotide or composition, when formulated into a composition with a delivery agent as described herein, can exhibit an improved volume of distribution (V_{dist}), e.g., reduced or targeted, relative to a composition lacking a delivery agent as described herein. The volume of distribution (Vdist) relates the amount of the drug in the body to the concentration of the drug in the blood or plasma. As used herein, the term “volume of distribution” refers to the fluid volume that would be required to contain the total amount of the drug in the body at the same concentration as in the blood or plasma: Vdist equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, Vdist can be used to determine a loading dose to achieve a steady state concentration. In some embodiments, the volume of distribution of the polynucleotide or composition when co-administered with a delivery agent as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Biological Effect

In one embodiment, the biological effect of the polynucleotide or composition delivered to the animals may be categorized by analyzing the protein expression in the animals. The protein expression may be determined from analyzing a biological sample collected from a mammal administered the polynucleotide or composition of the present invention.

### Detection of Polynucleotides by Mass Spectrometry

Mass spectrometry (MS) is an analytical technique that can provide structural and molecular mass/concentration information on molecules after their conversion to ions. The molecules are first ionized to acquire positive or negative charges and then they travel through the mass analyzer to arrive at different areas of the detector according to their mass/charge (m/z) ratio.

Mass spectrometry is performed using a mass spectrometer which includes an ion source for ionizing the fractionated sample and creating charged molecules for further analysis. For example ionization of the sample may be performed by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. The skilled artisan will understand that the choice of ionization method can be determined based on the analyte to be measured, type of sample, the type of detector, the choice of positive versus negative mode, etc.

After the sample has been ionized, the positively charged or negatively charged ions thereby created may be analyzed to determine a mass-to-charge ratio (i.e., m/z). Suitable analyzers for determining mass-to-charge ratios include quadropole analyzers, ion traps analyzers, and time-of-flight analyzers. The ions may be detected using several detection modes. For example, selected ions may be detected (i.e., using a selective ion monitoring mode (SIM)), or alternatively, ions may be detected using a scanning mode, e.g., multiple reaction monitoring (MRM) or selected reaction monitoring (SRM).

Liquid chromatography-multiple reaction monitoring (LC-MS/MRM) coupled with stable isotope labeled dilution of peptide standards has been shown to be an effective method for protein verification (e.g., Keshishian et al., Mol Cell Proteomics 2009 8: 2339-2349; Kuhn et al., Clin Chem 2009 55:1108-1117; Lopez et al., Clin Chem 2010 56:281-290; each of which are herein incorporated by reference in its entirety).

In one embodiment, a biological sample which may contain protein encoded by the polynucleotides of the present invention may be analyzed for protein using electrospray ionization. Electrospray ionization (ESI) mass spectrometry (ESIMS) uses electrical energy to aid in the transfer of ions from the solution to the gaseous phase before they are analyzed by mass spectrometry. Samples may be analyzed using methods known in the art (e.g., Ho et al., Clin Biochem Rev. 2003 24(1):3-12; herein incorporated by reference in its entirety).

In one embodiment, a biological sample which may contain protein encoded by the polynucleotides of the present invention may be analyzed for protein in a tandem ESIMS system (e.g., MS/MS). As non-limiting examples, the droplets may be analyzed using a product scan (or daughter scan) a precursor scan (parent scan) a neutral loss or a multiple reaction monitoring.

In one embodiment, a biological sample which may contain protein encoded by the polynucleotides of the present invention may be analyzed using matrix-assisted laser desorption/ionization (MALDI) mass spectrometry (MALDIMS). MALDI provides for the nondestructive vaporization and ionization of both large and small molecules, such as proteins.

In one embodiment, the analyte-matrix mixture may be formed using the dried-droplet method. A biologic sample is mixed with a matrix to create a saturated matrix solution where the matrix-to-sample ratio is approximately 5000:1. An aliquot (approximately 0.5-2.0 uL) of the saturated matrix solution is then allowed to dry to form the analyte-matrix mixture.

In one embodiment, the analyte-matrix mixture may be formed using the thin-layer method. A matrix homogeneous film is first formed and then the sample is then applied and may be absorbed by the matrix to form the analyte-matrix mixture.

In one embodiment, the analyte-matrix mixture may be formed using the thick-layer method. A matrix homogeneous film is formed with a nitro-cellulose matrix additive. Once the uniform nitro-cellulose matrix layer is obtained the sample is applied and absorbed into the matrix to form the analyte-matrix mixture.

In one embodiment, the analyte-matrix mixture may be formed using the sandwich method. A thin layer of matrix crystals is prepared as in the thin-layer method followed by the addition of droplets of aqueous trifluoroacetic acid, the sample and matrix. The sample is then absorbed into the matrix to form the analyte-matrix mixture.

### V. USES OF ANTIBODY COMPOSITIONS OF THE INVENTION THERAPEUTICS

### Production of Antibodies

In one embodiment of the invention, the polynucleotides of the antibody compositions, particularly the immunomodulatory agents or moieties, encode antibodies and/or fragments of such antibodies. These may be produced by any one of the methods described herein. The antibodies may be of any of the different subclasses or isotypes of immunoglobulin such as, but not limited to, IgA, IgE, IgD, IgG, or IgM, or any of the other subclasses. Exemplary antibody molecules and fragments that may be prepared according to the invention include, but are not limited to, immunoglobulin molecules, substantially intact immunoglobulin molecules and those portions of an immunoglobulin molecule that may contain the paratope. Such portion of antibodies that contain the paratope include, but are not limited to Fab, Fab′, F(ab′)₂, F(v) and those portions known in the art.

The polynucleotides of the invention may encode variant antibody polypeptides which may have a certain identity with a reference polypeptide sequence, or have a similar or dissimilar binding characteristic with the reference polypeptide sequence.

Antibodies used in the methods of the present invention may be antibodies comprising non-human antibody-derived variable region(s) sequences, derived from the immunized animals, and human antibody-derived constant region(s) sequences. In addition, they can also be humanized antibodies comprising complementary determining regions (CDRs) of non-human antibodies derived from the immunized animals and the framework regions (FRs) and constant regions derived from human antibodies. In another embodiment, the methods provided herein may be useful for enhancing antibody protein product yield in a cell culture process.

### Therapeutic Agents

The polynucleotides or compositions of the present invention can be used as therapeutic or prophylactic agents. They are provided for use in medicine. For example, an polynucleotide or composition described herein can be administered to a subject, wherein the polynucleotide is translated in vivo to produce a therapeutic or prophylactic polypeptide in the subject. Provided are compositions, methods, kits, and reagents for diagnosis, treatment or prevention of a disease or condition in humans and other mammals. The active therapeutic agents of the invention include polynucleotides or compositions, cells containing polynucleotides or compositions or polypeptides translated from the polynucleotides.

Provided herein are methods of inducing translation of a polypeptide (antibody, variant or fragment thereof) in a cell, tissue or organism using the polynucleotides described herein. Such translation can be in vivo, ex vivo, in culture, or in vitro. The cell, tissue or organism is contacted with an effective amount of a composition containing an antibody composition which contains a polynucletotide that has at least one a translatable region encoding the polypeptide of intereste (antibody).

An “effective amount” of the antibody composition is provided based, at least in part, on the target tissue, target cell type, means of administration, physical characteristics of the polynucleotide (e.g., size, and extent of modified nucleosides) and other components of the antibody, and other determinants.

Aspects of the invention are directed to methods of inducing in vivo translation of a polypeptide in a mammalian subject in need thereof. Therein, an effective amount of an antibody composition containing a polynucleotide that has at least one structural or chemical modification and a translatable region encoding the polypeptide (antibody) is administered to the subject using the delivery methods described herein. The polynucleotide is provided in an amount and under other conditions such that the polynucleotide is localized into a cell of the subject and the polypeptide is translated in the cell from the polynucleotide. The cell in which the polynucleotide is localized, or the tissue in which the cell is present, may be targeted with one or more than one rounds of antibody administration.

In certain embodiments, the administered antibody compositions comprising polynucleotides directs production of one or more polypeptides that provide a functional immune system-related activity which is substantially absent in the cell, tissue or organism in which the polypeptide is translated. For example, the missing functional activity may be enzymatic, structural, or gene regulatory in nature. In related embodiments, the administered polynucleotides direct production of one or more polypeptides that increases (e.g., synergistically) a functional activity related to the immune system which is present but substantially deficient in the cell in which the polypeptide is translated.

In other embodiments, the administered antibody compositions comprising polynucleotides directs production of one or more polypeptides that replace an immune related polypeptide (or multiple polypeptides) that is substantially absent in the cell in which the polypeptide is translated. Such absence may be due to genetic mutation of the encoding gene or regulatory pathway thereof. In some embodiments, the polypeptide increases the level of an endogenous protein in the cell to a desirable level; such an increase may induce or boost an immune response by bringing the level of the endogenous protein from a subnormal level to a normal level or from a normal level to a super-normal level.

Alternatively, the polypeptide functions to antagonize the activity of an endogenous protein present in, on the surface of, or secreted from the cell. Usually, the activity of the endogenous protein is deleterious to the subject or the subject's immune system; for example, due to mutation of the endogenous protein resulting in altered activity or localization.

Additionally, the polypeptide antagonizes, directly or indirectly, the activity of a biological moiety present in, on the surface of, or secreted from the cell. Examples of antagonized biological moieties include lipids (e.g., cholesterol), a lipoprotein (e.g., low density lipoprotein), a nucleic acid, a carbohydrate, a protein toxin such as shiga and tetanus toxins, or a small molecule toxin such as botulinum, cholera, and diphtheria toxins. Additionally, the antagonized biological molecule may be an endogenous protein that exhibits an undesirable activity, such as a cytotoxic or cytostatic activity.

The proteins described herein may be engineered for localization within the cell, potentially within a specific compartment such as the nucleus, or are engineered for secretion from the cell or translocation to the plasma membrane of the cell.

In some embodiments, polynucleotides of the invention and their encoded polypeptides in accordance with the present invention may be used for treatment of any of a variety of diseases, disorders, and/or conditions, including but not limited to one or more of the following: autoimmune disorders (e.g. diabetes, lupus, multiple sclerosis, psoriasis, rheumatoid arthritis); inflammatory disorders (e.g. arthritis, pelvic inflammatory disease); infectious diseases (e.g. viral infections (e.g., HIV, HCV, RSV), bacterial infections, fungal infections, sepsis); neurological disorders (e.g. Alzheimer's disease, Huntington's disease; autism; Duchenne muscular dystrophy); cardiovascular disorders (e.g. atherosclerosis, hypercholesterolemia, thrombosis, clotting disorders, angiogenic disorders such as macular degeneration); proliferative disorders (e.g. cancer, benign neoplasms); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); blood and lymphatic disorders (e.g. anemia, hemophilia); etc.

In another embodiment, the present invention provides a method for treating hematopoietic disorders, cardiovascular disease, oncology, diabetes, cystic fibrosis, neurological diseases, inborn errors of metabolism, skin and systemic disorders, and blindness. The identity of molecular targets to treat these specific diseases has been described (Templeton ed., Gene and Cell Therapy: Therapeutic Mechanisms and Strategies, 3^{rd} Edition, Bota Raton, Fla.: CRC Press; herein incorporated by reference in its entirety).

In certain embodiments, the administration may be local or systemic. In certain embodiments, the administration may be subcutaneous. In certain embodiments, the administration may be intravenous. In certain embodiments, the administration may be oral. In certain embodiments, the administration may be topical. In certain embodiments, the administration may be by inhalation. In certain embodiments, the administration may be rectal. In certain embodiments, the administration may be vaginal.

Other aspects of the present disclosure relate to transplantation of cells containing polynucleotides to a mammalian subject.

The subject to whom the therapeutic agent may be administered suffers from or may be at risk of developing a disease, disorder, or deleterious condition. Provided are methods of identifying, diagnosing, and classifying subjects on these bases, which may include clinical diagnosis, biomarker levels, genome-wide association studies (GWAS), and other methods known in the art.

### VI. KITS AND DEVICES

### Kits

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

In one aspect, the present invention provides kits comprising the antibody molecules (including any proteins or polynucleotides) of the invention. In one embodiment, the kit comprises one or more functional antibodies or function fragments thereof.

Said kits can be for protein production, comprising a first polynucleotides comprising a translatable region of an antibody. The kit may further comprise packaging and instructions and/or a delivery agent to form a formulation composition. The delivery agent may comprise a saline, a buffered solution, a lipidoid or any delivery agent disclosed herein.

In one embodiment, the buffer solution may include sodium chloride, calcium chloride, phosphate and/or EDTA. In another embodiment, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See e.g., U.S. Pub. No. 20120258046; herein incorporated by reference in its entirety). In a further embodiment, the buffer solutions may be precipitated or it may be lyophilized. The amount of each component may be varied to enable consistent, reproducible higher concentration saline or simple buffer formulations.

The components may also be varied in order to increase the stability of polynucleotides in the buffer solution over a period of time and/or under a variety of conditions. In one aspect, the present invention provides kits for protein production, comprising: a polynucleotide comprising a translatable region, provided in an amount effective to produce a desired amount of a protein encoded by the translatable region when introduced into a target cell; a second polynucleotide comprising an inhibitory nucleic acid, provided in an amount effective to substantially inhibit the innate immune response of the cell; and packaging and instructions.

In one aspect, the present invention provides kits for protein production, comprising a polynucleotide comprising a translatable region, wherein the polynucleotide exhibits reduced degradation by a cellular nuclease, and packaging and instructions.

In one aspect, the present invention provides kits for protein production, comprising a polynucleotide comprising a translatable region, wherein the polynucleotide exhibits reduced degradation by a cellular nuclease, and a mammalian cell suitable for translation of the translatable region of the first nucleic acid.

### Devices

The present invention provides for devices which may incorporate antibody compositions comprising polynucleotides that encode polypeptides of interest. These devices contain in a stable formulation the reagents to synthesize a polynucleotide in a formulation available to be immediately delivered to a subject in need thereof, such as a human patient.

Devices for administration may be employed to deliver the antibody compositions of the present invention according to single, multi- or split-dosing regimens taught herein. Such devices are taught in, for example, International Application PCT/US2013/30062 filed Mar. 9, 2013 (Attorney Docket Number M300), the contents of which are incorporated herein by reference in their entirety.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

According to the present invention, these multi-administration devices may be utilized to deliver the single, multi- or split doses contemplated herein. Such devices are taught for example in, International Application PCT/US2013/30062 filed Mar. 9, 2013 (Attorney Docket Number M300), the contents of which are incorporated herein by reference in their entirety.

In one embodiment, the antibody is administered subcutaneously or intramuscularly via at least 3 needles to three different, optionally adjacent, sites simultaneously, or within a 60 minutes period (e.g., administration to 4, 5, 6, 7, 8, 9, or 10 sites simultaneously or within a 60 minute period).

Methods and Devices Utilizing Catheters and/or Lumens

Methods and devices using catheters and lumens may be employed to administer the antibody compositions of the present invention on a single, multi- or split dosing schedule. Such methods and devices are described in International Application PCT/US2013/30062 filed Mar. 9, 2013 (Attorney Docket Number M300), the contents of which are incorporated herein by reference in their entirety.

### Methods and Devices Utilizing Electrical Current

Methods and devices utilizing electric current may be employed to deliver the antibody compositions of the present invention according to the single, multi- or split dosing regimens taught herein. Such methods and devices are described in International Application PCT/US2013/30062 filed Mar. 9, 2013 (Attorney Docket Number M300), the contents of which are incorporated herein by reference in their entirety.

### VII. DEFINITIONS

At various places in the present specification, substituents of compounds of the present disclosure are disclosed in groups or in ranges. It is specifically intended that the present disclosure include each and every individual subcombination of the members of such groups and ranges

About: As used herein, the term “about” means+/−10% of the recited value.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Adjuvant: As used herein, the term “adjuvant” means a substance that enhances a subject's immune response to an antigen.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antigen: As used herein, the term “antigen” refers to a substance or molecule that induces, elicits or triggers an immune response in a cell, tissue or organism. An antigen may originate either from the body, such as cancer antigen, or from the external environment, for instance, from infectious agents. Antigens may be, in whole or part, endogenous or exogenous peptides, proteins or polypeptides of interest or fragments thereof.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may effect the same outcome or a different outcome. The structure that produces the function may be the same or different. For example, bifunctional modified RNAs of the present invention may encode a cytotoxic peptide (a first function) while those nucleosides which comprise the encoding RNA are, in and of themselves, cytotoxic (second function). In this example, delivery of the bifunctional modified RNA to a cancer cell would produce not only a peptide or protein molecule which may ameliorate or treat the cancer but would also deliver a cytotoxic payload of nucleosides to the cell should degradation, instead of translation of the modified RNA, occur.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, a polynucleotide of the present invention may be considered biologically active if even a portion of the polynucleotides is biologically active or mimics an activity considered biologically relevant.

Chimera: As used herein, “chimera” is an entity having two or more incongruous or heterogeneous parts or regions.

Chimeric polynucleotide: As used herein, “chimeric polynucleotides” are those nucleic acid polymers having portions or regions which differ in size and/or chemical modification pattern, chemical modification position, chemical modification percent or chemical modification population and combinations of the foregoing.

Compound: As used herein, the term “compound,” is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Controlled Release: As used herein, the term “controlled release” refers to a pharmaceutical composition or compound release profile that conforms to a particular pattern of release to effect a therapeutic outcome.

Cyclic or Cyclized: As used herein, the term “cyclic” refers to the presence of a continuous loop. Cyclic molecules need not be circular, only joined to form an unbroken chain of subunits. Cyclic molecules such as the engineered RNA or mRNA of the present invention may be single units or multimers or comprise one or more components of a complex or higher order structure.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a polynucleotide to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Differentiated cell: As used herein, the term “differentiated cell” refers to any somatic cell that is not, in its native form, pluripotent. Differentiated cell also encompasses cells that are partially differentiated.

Differentiation: As used herein, the term “differentiation factor” refers to a developmental potential altering factor such as a protein, RNA or small molecule that can induce a cell to differentiate to a desired cell-type.

Differentiate: As used herein, “differentiate” refers to the process where an uncommitted or less committed cell acquires the features of a committed cell.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Dose splitting factor (DSF)—ratio of PUD of dose split treatment divided by PUD of total daily dose or single unit dose. The value is derived from comparison of dosing regimens groups.

Encapsulate: As used herein, the term “encapsulate” means to enclose, surround or encase.

Encoded protein cleavage signal: As used herein, “encoded protein cleavage signal” refers to the nucleotide sequence which encodes a protein cleavage signal.

Engineered: As used herein, embodiments of the invention are “engineered” when they are designed to have a feature or property, whether structural or chemical, that varies from a starting point, wild type or native molecule.

Effective Amount: As used herein, the term “effective amount” of an agentis that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least a polynucleotide encoding the polypeptide of interest and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

IVT Polynucleotide: As used herein, an “IVT polynucleotide” is a linear polynucleotide which may be made using only in vitro transcription (IVT) enzymatic synthesis methods.

Linker: As used herein, a “linker” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form polynucleotide multimers (e.g., through linkage of two or more chimeric polynucleotides molecules or IVT polynucleotides) or polynucleotides conjugates, or to provide a cleavage site to separate two or more polypeptides after translation, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof., Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the invention. Molecules may be modified in many ways including chemically, structurally, and functionally. In one embodiment, the polynucleotide molecules of the present invention are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Mucus: As used herein, “mucus” refers to the natural substance that is viscous and comprises mucin glycoproteins.

Naturally occurring: As used herein, “naturally occurring” means existing in nature without artificial aid.

Neutralizing antibody: As used herein, a “neutralizing antibody” refers to an antibody which binds to its antigen and defends a cell from an antigen or infectious agent by neutralizing or abolishing any biological activity it has.

Non-human vertebrate: As used herein, a “non human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Optionally substituted: Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

Part: As used herein, a “part” or “region” of a polynucleotide is defined as any portion of the polynucleotide which is less than the entire length of the polynucleotide.

Peptide: As used herein, “peptide” is less than or equal to 50 amino acids long, e.g., about 5, 10, 15, 20, 25, 30, 35, 40, 45, or 50 amino acids long.

Paratope: As used herein, a “paratope” refers to the antigen-binding site of an antibody.

Patient: As used herein, “patient” refers to a subject who may seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Polypeptide per unit drug (PUD): As used herein, a PUD or product per unit drug, is defined as a subdivided portion of total daily dose, usually 1 mg, pg, kg, etc., of a product (such as a polypeptide) as measured in body fluid or tissue, usually defined in concentration such as pmol/mL, mmol/mL, etc divided by the measure in the body fluid.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease. An “immune phrophylaxis” refers to a measure to produce active or passive immunity to prevent the spread of disease.

Protein cleavage site: As used herein, “protein cleavage site” refers to a site where controlled cleavage of the amino acid chain can be accomplished by chemical, enzymatic or photochemical means.

Protein cleavage signal: As used herein “protein cleavage signal” refers to at least one amino acid that flags or marks a polypeptide for cleavage.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Pseudouridine: As used herein, pseudouridine refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m¹ψ), 1-methyl-4-thio-pseudouridine (m¹s⁴ψ)4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine (acp³ ψ), and 2′-O-methyl-pseudouridine (ψm).

Purified: As used herein, “purify,” “purified,” “purification” means to make substantially pure or clear from unwanted components, material defilement, admixture or imperfection.

Repeated transfection or repeated dose: As used herein, the term “repeated transfection” or “dose” refers to transfection of the same cell culture with a polynucleotide a plurality of times. The cell culture can be transfected at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times at least 18 times, at least 19 times, at least 20 times, at least 25 times, at least 30 times, at least 35 times, at least 40 times, at least 45 times, at least 50 times or more.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transcription: As used herein, the term “transcription” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1

### Manufacture of Polynucleotides

According to the present invention, the manufacture of polynucleotides and or parts or regions thereof may be accomplished utilizing the methods taught in U.S. Ser. No. 61/800,049 filed Mar. 15, 2013 entitled “Manufacturing Methods for Production of RNA Transcripts” (Attorney Docket number M500), the contents of which is incorporated herein by reference in its entirety.

Purification methods may include those taught in U.S. Ser. No. 61/799,872 filed Mar. 15, 2013 entitled “Methods of removing DNA fragments in mRNA production” (Attorney Docket number M501); U.S. Ser. No. 61/794,842 filed Mar. 15, 2013, entitled “Ribonucleic acid purification” (Attorney Docket number M502), each of which is incorporated herein by reference in its entirety.

Detection and characterization methods of the polynucleotides may be performed as taught in U.S. Ser. No. 61/798,945 filed Mar. 15, 2013 entitled “Characterization of mRNA Molecules (Attorney Docket number M505), the contents of which are incorporated herein by reference in their entirety.

Characterization of the polynucleotides of the invention may be accomplished using a procedure selected from the group consisting of polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA transcript. Such methods are taught in, for example, U.S. Ser. No. 61/799,905 filed Mar. 15, 2013 entitled “Analysis of mRNA Heterogeneity and Stability” (Attorney Docket number M506) and U.S. Ser. No. 61/800,110 filed Mar. 15, 2013 entitled “Ion Exchange Purification of mRNA” (Attorney Docket number M507) the contents of each of which is incorporated herein by reference in its entirety.

### Example 2

### Synthesis of Polynucleotides

Enzymatic (IVT), solid-phase, liquid-phase, combined synthetic methods, small region synthesis, and ligation methods are taught in for example copending application U.S. 61/912,635 filed Dec. 6, 2013 (Attorney Docket Number M073.60), the contents of which are incorporated herein by reference in their entirety, and may be utilized to manufacture the polynucleotides of the present invention.

### Example 3

### Method of Screening for Protein Expression

A. Electrospray Ionization

A biological sample which may contain proteins encoded by a polynucleotide administered to the subject is prepared and analyzed according to the manufacturer protocol for electrospray ionization (ESI) using 1, 2, 3 or 4 mass analyzers. A biologic sample may also be analyzed using a tandem ESI mass spectrometry system.

Patterns of protein fragments, or whole proteins, are compared to known controls for a given protein and identity is determined by comparison.

B. Matrix-Assisted Laser Desorption/Ionization

A biological sample which may contain proteins encoded by one or more polynucleotides administered to the subject is prepared and analyzed according to the manufacturer protocol for matrix-assisted laser desorption/ionization (MALDI).

Patterns of protein fragments, or whole proteins, are compared to known controls for a given protein and identity is determined by comparison.

C. Liquid Chromatography-Mass Spectrometry-Mass Spectrometry

A biological sample, which may contain proteins encoded by one or more polynucleotides, may be treated with a trypsin enzyme to digest the proteins contained within. The resulting peptides are analyzed by liquid chromatography-mass spectrometry-mass spectrometry (LC/MS/MS). The peptides are fragmented in the mass spectrometer to yield diagnostic patterns that can be matched to protein sequence databases via computer algorithms. The digested sample may be diluted to achieve 1 ng or less starting material for a given protein. Biological samples containing a simple buffer background (e.g. water or volatile salts) are amenable to direct in-solution digest; more complex backgrounds (e.g. detergent, non-volatile salts, glycerol) require an additional clean-up step to facilitate the sample analysis.

Patterns of protein fragments, or whole proteins, are compared to known controls for a given protein and identity is determined by comparison.

### Example 4

### Method of Screening for Antibody Polypeptide Function

Cells, tissues or a subject is treated with the polynucleotide or composition of the invention at a dose equivalent to or less than the dose typically administered for the parent antibody. The cells, tissue or subject is then assayed for phenotypic outcomes associated with the parent antibody in similar manner as is known in the art for the parent antibody or those described herein.

Improvements in outcomes or side-effects are expected when the polynucleotide is administered in comparison to administration of the parent antibody

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

It is to be understood that the words which have been used are words of description rather than limitation, and that changes may be made within the purview of the appended claims without departing from the true scope and spirit of the invention in its broader aspects.

1. A composition comprising,
(a) a polynucleotide encoding at least one polypeptide, wherein the polypeptide is selected from the complete antibody, a variant, a fragment and combinations thereof and wherein said polynucleotide comprises at least one chemical modification and
(b) a pharmaceutically acceptable carrier or excipient.

2. The composition of claim 1, wherein the fragment comprises the (complementary determining regions) CDRs of the antibody.

3. The composition of claim 2, wherein the polynucleotide encoding the at least one polypeptide encodes a member selected from the group consisting of an intrabody, a bicistronic antibody, a pseudobicistronic antibody, a single domain antibody, a single chain variable fragment (scFv) antibody, and a bispecific antibody.

4. The composition of claim 1, wherein the polynucleotide further encodes at least one cleavage site or linker.

5. The composition of claim 4, wherein the linker is a GS linker.

6. The composition of claim 4, wherein the polynucleotide encodes at least one cleavage site and the at least one cleavage site is a proteolytic cleavage site.

7. The composition of claim 6, wherein the proteolytic cleavage site is an RKR furin cleavage site.

8. The composition of claim 1, wherein the chemical modification is 1-methylpseudouridine.

9. A method of producing an antibody in a cell, tissue or organism, comprising contacting said cell tissue or organism with a composition comprising,
(a) a polynucleotide encoding at least one polypeptide, wherein the polypeptide is selected from the complete antibody, a variant, a fragment and combinations thereof and wherein said polynucleotide comprises at least one chemical modification and
(b) a pharmaceutically acceptable carrier or excipient.

10. The method of claim 9, wherein the fragment comprises the (complementary determining regions) CDRs of the antibody.

11. The method of claim 10, wherein the polynucleotide encoding the at least one polypeptide encodes a member selected from the group consisting of an intrabody, a bicistronic antibody, a pseudobicistronic antibody, a single domain antibody, a single chain variable fragment (scFv) antibody, and a bispecific antibody.

12. The method of claim 9, wherein the polynucleotide further encodes at least one cleavage site or linker.

13. The method of claim 12, wherein the linker is a GS linker.

14. The method of claim 12, wherein the polynucleotide encodes at least one cleavage site and the at least one cleavage site is a proteolytic cleavage site.

15. The method of claim 14, wherein the proteolytic cleavage site is an RKR furin cleavage site.

16. The method of claim 9, wherein the chemical modification is 1-methylpseudouridine.


SEQUENCE LISTING (395 sequences)

SEQ ID NO: 1 - protein, 450 aa, Artificial Sequence
  Description of Artificial Sequence: Synthetic polypeptide
EVQLQQSGAELVKPGASVKLSCTASGFNIKDTYVHWVKQRPEQGLEWIGRIDPANGYTKYDPKFQGKATITADTSSNTAYLQLSSLTSEDTAVYYCVRPLYDYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Description of Artificial Sequence: Synthetic polypeptide
DILMTQSPSSMSVSLGDTVSITCHASQGISSNIGWLQQKPGKSFMGLIYYGTNLVDGVPSRFSGSGSGADYSLTISSLDSEDFADYYCVQYAQLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 224 aa, Artificial Sequence
  Description of Artificial Sequence: Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 4 - protein, 214 aa, Artificial Sequence
  Description of Artificial Sequence: Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 121 aa, Artificial Sequence [sequence omitted]

SEQ ID NO: 6 - protein, 107 aa, Artificial Sequence [sequence omitted]

SEQ ID NO: 7 - protein, 451 aa, Artificial Sequence
  Description of Artificial Sequence: Synthetic polypeptide
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 211 aa, Artificial Sequence
  Description of Artificial Sequence: Synthetic polypeptide
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 9 - protein, 219 aa, Artificial Sequence
  Description of Artificial Sequence: Synthetic polypeptide
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 10 - protein, 214 aa, Artificial Sequence
  Description of Artificial Sequence: Synthetic polypeptide
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 437 aa, Artificial Sequence
  Description of Artificial Sequence: Synthetic polypeptide
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence; identical to SEQ ID NO: 10
  Description of Artificial Sequence: Synthetic polypeptide

SEQ ID NO: 13 - protein, 220 aa, Artificial Sequence
  Description of Artificial Sequence: Synthetic polypeptide
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVE

SEQ ID NO: 14 - protein, 211 aa, Artificial Sequence; identical to SEQ ID NO: 8
  Description of Artificial Sequence: Synthetic polypeptide

SEQ ID NO: 15 - protein, 446 aa, Artificial Sequence
  Description of Artificial Sequence: Synthetic polypeptide
QLQQSGTVLARPGASVKMSCKASGYSFTRYWMHWIKQRPGQGLEWIGAIYPGNSDTSYNQKFEGKAKLTAVTSASTAYMELSSLTHEDSAVYYCSRDYGYYFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 210 aa, Artificial Sequence
  Description of Artificial Sequence: Synthetic polypeptide
QIVSTQSPAIMSASPGEKVTMTCSASSSRSYMQWYQQKPGTSPKRWIYDTSKLASGVPARFSGSGSGTSYSLTISSMEAEDAATYYCHQRSSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 17 - protein, 249 aa, Artificial Sequence
  Description of Artificial Sequence: Synthetic polypeptide
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSGGGGSGGGGSGGGGSAFSSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLG

SEQ ID NO: 18 - protein, 123 aa, Artificial Sequence
  Description of Artificial Sequence: Synthetic polypeptide
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSS

SEQ ID NO: 19 - protein, 109 aa, Artificial Sequence
  Description of Artificial Sequence: Synthetic polypeptide
SSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLG

SEQ ID NO: 20 - protein, 249 aa, Artificial Sequence
  Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGPRYYDILTGYRYNWFDPWGRGTLVTVSSGGGGSGGGGSGGGGSDIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIKR

SEQ ID NO: 21 - protein, 126 aa, Artificial Sequence
  Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGPRYYDILTGYRYNWFDPWGRGTLVTVSS

SEQ ID NO: 22 - protein, 251 aa, Artificial Sequence
  Description of Artificial Sequence: Synthetic polypeptide
QLQLQESGPGLVKPSETLSLTCTVSGGFISSRTSYWGWIRQPPGKGPEWIGNIYYTGKTYYSPSLKSRVTISVDTSKNQLSLKLNSVTAADTAVYYCARAGYDLLTGYPFYFDSWGKGTLVTVSSGGGGSGGGGSGGGGSALEIVLTQSPATLSLSPGERATLSCRASQSVSSYLAWYQQKPGQAPRLLIYDASNRATGIPARFSGSGSGTDFTLTISSLEPEDFAVYYCQQRSNWPFLTFGGGTKVEIKR

SEQ ID NO: 23 - protein, 109 aa, Artificial Sequence
  Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPFLTFGGGTKVEIKR

SEQ ID NO: 24 - protein, 453 aa, Artificial Sequence
  Description of Artificial Sequence: Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 214 aa, Artificial Sequence
  Description of Artificial Sequence: Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEEDSKDSTYSLSSTLTLSKADYEKHKVYACEVTHQGLSSPVTKSFNRGEC

SEQ ID NO: 26 - protein, 111 aa, Artificial Sequence
  Description of Artificial Sequence: Synthetic polypeptide
DIVLTQSPATLSLSPGERATLSCRASQSVDFDGDSYMNWYQQKPGQPPKVLIYAASNLESGIPARFSGSGSGTDFTLTISSLQPEDFATYYCQQSNEDPWTFGGGTKVEIK

SEQ ID NO: 27 - protein, 111 aa, Artificial Sequence
  Description of Artificial Sequence: Synthetic polypeptide
DIVLTQSPSSLSASVGDRVTITCRASQSVDFDGDSYMNWYQQKPGQPPKVLIYAASNLESGIPARFSGSGSGTDFTLTISSLQPEDFATYYCQQSNEDPWTFGGGTKVEIK

SEQ ID NO: 28 - protein, 111 aa, Artificial Sequence
  Description of Artificial Sequence: Synthetic polypeptide
DIVLTQSPDSLAVSLGERATINCKASQSVDFDGDSYMNWYQQKPGQPPKVLIYAASNLESGIPARFSGSGSGTDFTLTINSLEAEDAATYYCQQSNEDPWTFGGGTKVEIK

SEQ ID NO: 29 - protein, 117 aa, Artificial Sequence
  Description of Artificial Sequence: Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 30 - protein, 117 aa, Artificial Sequence
  Description of Artificial Sequence: Synthetic polypeptide
QIQLVESGGGLVKPGGSLRLSCAASGYTFTDYYITWVRQAPGQGLEWMGWIYPGSGNTKYNEKFQGRVTMTVDTSTSTAYMELSSLRSEDTAVYFCANYGNYWFAYWGQGTLVTVSS

SEQ ID NO: 31 - protein, 117 aa, Artificial Sequence
  Description of Artificial Sequence: Synthetic polypeptide
QIQLVQSGPEVKKPGASVKVSCKASGYTFTDYYITWVRQAPGQGLEWMGWIYPGSGNTKYNEKFQGRFVFSVDTSASTAYLQISSLKAEDTAVYFCANYGNYWFAYWGQGTLVTVSS

SEQ ID NO: 32 - protein, 330 aa, Artificial Sequence [sequence omitted]

SEQ ID NO: 33 - protein, 448 aa, Artificial Sequence
  Description of Artificial Sequence: Synthetic polypeptide
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 394 aa, Artificial Sequence [sequence omitted]

SEQ ID NO: 35 - protein, 118 aa, Artificial Sequence [sequence omitted]

SEQ ID NO: 36 - protein, 107 aa, Artificial Sequence [sequence omitted]

SEQ ID NO: 37 - protein, 452 aa, Artificial Sequence
  Description of Artificial Sequence: Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 213 aa, Artificial Sequence
  Description of Artificial Sequence: Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 39 - protein, 229 aa, Artificial Sequence
  Description of Artificial Sequence: Synthetic polypeptide
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 40 - protein, 214 aa, Artificial Sequence
  Description of Artificial Sequence: Synthetic polypeptide
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 116 aa, Artificial Sequence
  Description of Artificial Sequence: Synthetic polypeptide
QVQLQQSGAELAKPGASVKMSCKASGYTFTSYRMHWVKQRPGQGLEWIGYINPSTGYTEYNQKFKDKATLTADKSSSTAYMQLSSLTFEDSAVYYCARGGGVFDYWGQGTTLTVSS

SEQ ID NO: 42 - protein, 106 aa, Artificial Sequence
  Description of Artificial Sequence: Synthetic polypeptide
QIVLTQSPAIMSASPGEKVTITCSASSSISYMHWFQQKPGTSPKLWIYTTSNLASGVPARFSGSGSGTSYSLTISRMEAEDAATYYCHQRSTYPLTFGSGTKLELK

SEQ ID NO: 43 - protein, 5 aa, Artificial Sequence
  Description of Artificial Sequence: Synthetic peptide
SYRMH

SEQ ID NO: 44 - protein, 17 aa, Artificial Sequence
  Description of Artificial Sequence: Synthetic peptide
YINPSTGYTEYNQKFKD

SEQ ID NO: 45 - protein, 7 aa, Artificial Sequence
  Description of Artificial Sequence: Synthetic peptide
GGGVFDY

SEQ ID NO: 46 - protein, 10 aa, Artificial Sequence
  Description of Artificial Sequence: Synthetic peptide
SASSSISYMH

SEQ ID NO: 47 - protein, 7 aa, Artificial Sequence
  Description of Artificial Sequence: Synthetic peptide
TTSNLAS

SEQ ID NO: 48 - protein, 9 aa, Artificial Sequence
  Description of Artificial Sequence: Synthetic peptide
HQRSTYPLT

SEQ ID NO: 49 - protein, 467 aa, Artificial Sequence
  Description of Artificial Sequence: Synthetic polypeptide
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 50 - protein, 235 aa, Artificial Sequence
  Description of Artificial Sequence: Synthetic polypeptide
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 121 aa, Artificial Sequence
  Description of Artificial Sequence: Synthetic polypeptide
VQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 52 - protein, 108 aa, Artificial Sequence
  Description of Artificial Sequence: Synthetic polypeptide
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 53 - protein, 122 aa, Artificial Sequence
  Description of Artificial Sequence: Synthetic polypeptide
QVQLQQSGAELMKPGASVKMSCKATGYIFSNYWIQWIKQRPGHGLEWIGEILPGSGSTEYTENFKDKAAFTADTSSNTAYMQLSSLTSEDSAVYYCARYFFGSSPNWYFDVWGAGTTVTVSS

SEQ ID NO: 54 - protein, 107 aa, Artificial Sequence
  Description of Artificial Sequence: Synthetic polypeptide
DIQMTQSPASLSASVGETVTITCGASENIYGALNWYQRKQGKSPQLLIYGATNLADGMSSRFSGSGSGRQYYLKISSLHPDDVATYYCQNVLNTPLTFGAGTKLELK

SEQ ID NO: 55 - protein, 5 aa, Artificial Sequence
  Description of Artificial Sequence: Synthetic peptide
NYWIQ

SEQ ID NO: 56 - protein, 17 aa, Artificial Sequence
  Description of Artificial Sequence: Synthetic peptide
EILPGSGSTEYTENFKD

SEQ ID NO: 57 - protein, 13 aa, Artificial Sequence
  Description of Artificial Sequence: Synthetic peptide
YFFGSSPNWYFDV

SEQ ID NO: 58 - protein, 11 aa, Artificial Sequence
  Description of Artificial Sequence: Synthetic peptide
GASENIYGALN

SEQ ID NO: 59 - protein, 7 aa, Artificial Sequence
  Description of Artificial Sequence: Synthetic peptide
GATNLAD

SEQ ID NO: 60 - protein, 7 aa, Artificial Sequence
  Description of Artificial Sequence: Synthetic peptide
VLNTPLT

SEQ ID NO: 61 - protein, 448 aa, Artificial Sequence
  Description of Artificial Sequence: Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 214 aa, Artificial Sequence [sequence omitted]

SEQ ID NO: 63 - protein, 126 aa, Artificial Sequence
  Description of Artificial Sequence: Synthetic polypeptide
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 64 - protein, 108 aa, Artificial Sequence
  Description of Artificial Sequence: Synthetic polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 65 - protein, 10 aa, Artificial Sequence
  Description of Artificial Sequence: Synthetic peptide
GFIFSSYAMH

SEQ ID NO: 66 - protein, 17 aa, Artificial Sequence
  Description of Artificial Sequence: Synthetic peptide
FMSYDGSNKKYADSVKG

SEQ ID NO: 67 - protein, 17 aa, Artificial Sequence
  Description of Artificial Sequence: Synthetic peptide
DRGIAAGGNYYYYGMDV

SEQ ID NO: 68 - protein, 11 aa, Artificial Sequence
  Description of Artificial Sequence: Synthetic peptide
RASQSVYSYLA

SEQ ID NO: 69 - protein, 7 aa, Artificial Sequence
  Description of Artificial Sequence: Synthetic peptide
DASNRAT

SEQ ID NO: 70 - protein, 10 aa, Artificial Sequence
  Description of Artificial Sequence: Synthetic peptide
QQRSNWPPFT

SEQ ID NO: 71 - protein, 443 aa, Artificial Sequence
  Description of Artificial Sequence: Synthetic polypeptide
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSR

SEQ ID NO: 72 - protein, 209 aa, Artificial Sequence
  Description of Artificial Sequence: Synthetic polypeptide
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRADAAPTVFIFPPSDEQLKSGTASVVCLLNNFYPREAKVQWKVDNALQSGNSQESVTEQDSKDSTYSLSSTLTLSKADYEKHKVYACEVTHQGLSSPVTKSFN

SEQ ID NO: 73 - protein, 140 aa, Artificial Sequence
  Description of Artificial Sequence: Synthetic polypeptide
MGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 74 - protein, 128 aa, Artificial Sequence
  Description of Artificial Sequence: Synthetic polypeptide
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 75 - protein, 121 aa, Artificial Sequence [sequence omitted]

SEQ ID NO: 76 - protein, 125 aa, Artificial Sequence
  Description of Artificial Sequence: Synthetic polypeptide
QVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 77 - protein, 120 aa, Artificial Sequence
  Description of Artificial Sequence: Synthetic polypeptide
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSS

SEQ ID NO: 78 - protein, 107 aa, Artificial Sequence
  Description of Artificial Sequence: Synthetic polypeptide
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 79 - DNA, 355 bp, Artificial Sequence
  Description of Artificial Sequence: Synthetic polynucleotide
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcag

SEQ ID NO: 80 - protein, 118 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 81 - DNA, 325 bp, Artificial Sequence
  Description of Artificial Sequence: Synthetic polynucleotide
gaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaac

SEQ ID NO: 82 - protein, 108 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 83 - protein, 5 aa, Artificial Sequence
  Description of Artificial Sequence: Synthetic peptide
SYTMH

SEQ ID NO: 84 - protein, 17 aa, Artificial Sequence
  Description of Artificial Sequence: Synthetic peptide
FISYDGNNKYYADSVKG

SEQ ID NO: 85 - protein, 9 aa, Artificial Sequence
  Description of Artificial Sequence: Synthetic peptide
TGWLGPFDY

SEQ ID NO: 86 - protein, 12 aa, Artificial Sequence
  Description of Artificial Sequence: Synthetic peptide
RASQSVGSSYLA

SEQ ID NO: 87 - protein, 7 aa, Artificial Sequence
  Description of Artificial Sequence: Synthetic peptide
GAFSRAT

SEQ ID NO: 88 - protein, 9 aa, Artificial Sequence
  Description of Artificial Sequence: Synthetic peptide
QQYGSSPWT

SEQ ID NO: 89 - protein, 450 aa, Artificial Sequence
  Description of Artificial Sequence: Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 213 aa, Artificial Sequence
  Description of Artificial Sequence: Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 91 - protein, 140 aa, Artificial Sequence
  Description of Artificial Sequence: Synthetic polypeptide
MKCSWVMFFLMAVVTGVNSQVQLQQSGAELVKPGASVKLSCTASGFNIKDTYIHCVKQRPEQGLEWIGRIDPANGYTKYDPKFQGKATITADTSSNTAYLQLSSLTSEDTAVYFCAREGYYGNYGVYAMDYWGQGTSVTV

SEQ ID NO: 92 - protein, 126 aa, Artificial Sequence
  Description of Artificial Sequence: Synthetic polypeptide
MRPSIQFLGLLLFWLHGAQCDIQMTQSPSSLSASLGGKVTITCKTSQDINKYMAWYQHKPGKRPRLLIHYTSALQPGIPSRFSGSGSGRDYSFNISNLQPQDIATYYCLQYDNLWTFGGGTKLQIK

SEQ ID NO: 93 - protein, 119 aa, Artificial Sequence
  Description of Artificial Sequence: Synthetic polypeptide
QVQLVQSGAQVKKPGASVKVSCKASGYTFTSYAMHWVRQAPGQRLQWMGWINAGNGNTKYSQKFQGRVTITRDTSASTAYMELSSLRSEDTAVYYCARGGYYGSGSNYWGQGTLVTVSS

SEQ ID NO: 94 - protein, 106 aa, Artificial Sequence
  Description of Artificial Sequence: Synthetic polypeptide
DIQMTQSPSSLSASVGDRVTITCQASQDIIKYLNWYQQTPGKAPKLLIYEASNLQAGVPSRFSGSGSGTDYTFTISSLQPEDIATYCQQYQSLPYTFGQGTKLQIT

SEQ ID NO: 95 - protein, 123 aa, Artificial Sequence [sequence omitted]

SEQ ID NO: 96 - protein, 119 aa, Artificial Sequence
  Description of Artificial Sequence: Synthetic polypeptide
QVQLVQSGAEVKKPGASVKVSCKASGFNIKSTAMHWVRQAPGQGLEWMGWINAGNGNTKYSQKFQGRVTITADTSASTAYMELSSLRSEDTAVYYCARGGYYGSGSNYWGQGTLVTVSS

SEQ ID NO: 97 - protein, 119 aa, Artificial Sequence
  Description of Artificial Sequence: Synthetic polypeptide
QVQLVQSGAEVKKPGASVKVSCKASGFNIKSYAMHWVRQAPGQRLEWMGWINAGNGNTKYSQKFQGRVTITADTSASTAYMELSSLRSEDTAVYYCARGGYFGSGSNYWGQGTLVTVSS

SEQ ID NO: 98 - protein, 106 aa, Artificial Sequence
  Description of Artificial Sequence: Synthetic polypeptide
DIQMTQSPSSLSASVGDRVTITCKTSQDINKYMAWYQQTPGKAPRLLIHYTSALQPGIPSRFSGSGSGRDYTFTISSLQPQDIATYYCLQYDNLWTFGQGTKVEIK

SEQ ID NO: 99 - protein, 107 aa, Artificial Sequence
  Description of Artificial Sequence: Synthetic polypeptide
DIQMTQSPSSLSASVGDRVTITCQASQDIIKYLNWYQQTPGKAPRLLIYEASNLQAGIPSRFSGSGSGRDYTFTISSLQPQDIATYYCQQYQSLPYTFGQGTKLQIT

SEQ ID NO: 100 - protein, 141 aa, Artificial Sequence
  Description of Artificial Sequence: Synthetic polypeptide
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 101 - DNA, 423 bp, Artificial Sequence
  Description of Artificial Sequence: Synthetic polynucleotide
atggagttgggactgagctggattttccttttggctattttaaaaggtgtccagtgtgagtgcagctggtggagtctgggggaggcttggtacagcctggcaggtccctgagactctcctgtgcagcctctggattcacctttaatgattatgccatgcactgggtccggcaagctccagggaagggcctggagtgggtctcaactattagttggaatagtggttccataggctatgcggactctgtgaagggccgattcaccatctccagagacaacgccaagaagtccctgtatctgcaaatgaacagtctgagagctgaggacacggccttgtattactgtgcaaaagatatacagtacggcaactactactacggtatggacgtctggggccaagggaccacggtcaccgtctcctcag

SEQ ID NO: 102 - protein, 127 aa, Artificial Sequence
  Description of Artificial Sequence: Synthetic polypeptide
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 103 - DNA, 382 bp, Artificial Sequence
  Description of Artificial Sequence: Synthetic polynucleotide
atggaagccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgatcaccttcggccaagggacacgactggagattaaac

SEQ ID NO: 104 - protein, 6 aa, Artificial Sequence
  Description of Artificial Sequence: Synthetic peptide
NDYAMH

SEQ ID NO: 105 - protein, 17 aa, Artificial Sequence
  Description of Artificial Sequence: Synthetic peptide
TISWNSGSIGYADSVKG

SEQ ID NO: 106 - protein, 13 aa, Artificial Sequence
  Description of Artificial Sequence: Synthetic peptide
DIQYGNYYYGMDV

SEQ ID NO: 107 - protein, 11 aa, Artificial Sequence
  Description of Artificial Sequence: Synthetic peptide
RASQSVSSYLA

SEQ ID NO: 108 - protein, 7 aa, Artificial Sequence
  Description of Artificial Sequence: Synthetic peptide
DASNRAT

SEQ ID NO: 109 - protein, 9 aa, Artificial Sequence
  Description of Artificial Sequence: Synthetic peptide
QQRSNWPIT

SEQ ID NO: 110 - protein, 447 aa, Artificial Sequence
  Description of Artificial Sequence: Synthetic polypeptide
EVQLVESGGGLVQPGGSLRLSCAVSGYSITSGYSWNWIRQAPGKGLEWVASITYDGSTNYADSVKGRFTISRDDSKNTFYLQMNSLRAEDTAVYYCARGSHYFGHWHFAVWGQGTLVTVSSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 215 aa, Artificial Sequence
  Description of Artificial Sequence: Synthetic polypeptide
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 112 - protein, 95 aa, Artificial Sequence
  Description of Artificial Sequence: Synthetic polypeptide
VSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTSS

SEQ ID NO: 113 - protein, 105 aa, Artificial Sequence
  Description of Artificial Sequence: Synthetic polypeptide
TITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQ

SEQ ID NO: 114 - protein, 77 aa, Artificial Sequence
  Description of Artificial Sequence: Synthetic polypeptide
VVSGGSVSSGSYYWSWIRQPPGKGLEWIGYIYYSGSTNYNPSLKSRVTISVDTSKNQFSLKLSSVTAADTAVYYCAR

SEQ ID NO: 115 - protein, 76 aa, Artificial Sequence
  Description of Artificial Sequence: Synthetic polypeptide
TITCQASQDISNYLNWYQQKPGKAPKLLIYDASNLETGVPSRFSGSGSGTDFTFTISSLQPEDIATYYCQQYDNLP

SEQ ID NO: 116 - protein, 213 aa, Artificial Sequence
  Description of Artificial Sequence: Synthetic polypeptide
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 117 - protein, 218 aa, Artificial Sequence
  Description of Artificial Sequence: Synthetic polypeptide
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSGTAALGCLVKDYFPEPVTVSWNSGALTSGVHTFPAVLQSSGLYSLSSVVTVPSSSLGTQTYICNVNHKPSNTKVDKKVEPK

SEQ ID NO: 118 - protein, 218 aa, Artificial Sequence; identical to SEQ ID NO: 117
  Description of Artificial Sequence: Synthetic polypeptide

SEQ ID NO: 119 - protein, 213 aa, Artificial Sequence; identical to SEQ ID NO: 116
  Description of Artificial Sequence: Synthetic polypeptide

SEQ ID NO: 120 - protein, 451 aa, Artificial Sequence
  Description of Artificial Sequence: Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 213 aa, Artificial Sequence
  Description of Artificial Sequence: Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 139 aa, Artificial Sequence
  Description of Artificial Sequence: Synthetic polypeptide
MRVLILLWLFTAFPGILSDVQLQESGPVLVKPSQSLSLTCTVTGYSITSDHAWSWIRQFPGNKLEWMGYISYSGITTYNPSLKSRISITRDTRDTSNQFFLQLNSVTTGDTSTYYCARSLARTAMDYWGQGTTSVTVSS

SEQ ID NO: 123 - protein, 124 aa, Artificial Sequence
  Description of Artificial Sequence: Synthetic polypeptide
MVSSAQFLGLLLLCFQGTRCDIQMTQTTSSLSASLGDRVTISCRASQDISSYLNWYQQKPDGTIKLLIYYTSRLHSGVPSRFSGSGTDYSLTINNLEQEDIATYFCQQGNTLPYTFGGTKLEIN

SEQ ID NO: 124 - protein, 447 aa, Artificial Sequence
  Description of Artificial Sequence: Synthetic polypeptide
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 210 aa, Artificial Sequence
  Description of Artificial Sequence: Synthetic polypeptide
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 126 - protein, 451 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 214 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 214 aa, Artificial Sequence; identical to SEQ ID NO: 127
  Description of Artificial Sequence: Synthetic polypeptide

SEQ ID NO: 129 - protein, 220 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 130 - protein, 450 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDTPPPCPRCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 214 aa, Artificial Sequence; identical to SEQ ID NO: 127
  Description of Artificial Sequence: Synthetic polypeptide

SEQ ID NO: 132 - protein, 109 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 133 - protein, 12 aa, Artificial Sequence
  Description of Artificial Sequence: Synthetic peptide
RASQDVNTAVAW

SEQ ID NO: 134 - protein, 7 aa, Artificial Sequence
  Description of Artificial Sequence: Synthetic peptide
SASFLYS

SEQ ID NO: 135 - protein, 9 aa, Artificial Sequence
  Description of Artificial Sequence: Synthetic peptide
QQHYTTPPT

SEQ ID NO: 136 - protein, 119 aa, Artificial Sequence
  Description of Artificial Sequence: Synthetic polypeptide
VQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 137 - protein, 10 aa, Artificial Sequence
  Description of Artificial Sequence: Synthetic peptide
GRNIKDTYIH

SEQ ID NO: 138 - protein, 17 aa, Artificial Sequence
  Description of Artificial Sequence: Synthetic peptide
RIYPTNGYTRYADSVKG

SEQ ID NO: 139 - protein, 11 aa, Artificial Sequence
  Description of Artificial Sequence: Synthetic peptide
WGGDGFYAMDY

SEQ ID NO: 140 - protein, 210 aa, Artificial Sequence
  Description of Artificial Sequence: Synthetic polypeptide
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLEVKGFYPSDIAVEWESNGQPENNYKTTPPVLDSDGSFFLYSKRLTVDKSRWQQGNVFSCSVMHEALHNHYTQKSLSL

SEQ ID NO: 141 - protein, 211 aa, Artificial Sequence [sequence omitted]

SEQ ID NO: 142 - protein, 5 aa, Artificial Sequence
  Description of Artificial Sequence: Synthetic peptide
TYWLG

SEQ ID NO: 143 - protein, 16 aa, Artificial Sequence
  Description of Artificial Sequence: Synthetic peptide
IMSPVDSDIRYSPSFQ

SEQ ID NO: 144 - protein, 10 aa, Artificial Sequence
  Description of Artificial Sequence: Synthetic peptide
RRPGQGYFDF

SEQ ID NO: 145 - protein, 11 aa, Artificial Sequence
  Description of Artificial Sequence: Synthetic peptide
RASQGISSWLA

SEQ ID NO: 146 - protein, 7 aa, Artificial Sequence
  Description of Artificial Sequence: Synthetic peptide
AASSLQS

SEQ ID NO: 147 - protein, 9 aa, Artificial Sequence
  Description of Artificial Sequence: Synthetic peptide
QQYNIYPYT

SEQ ID NO: 148 - protein, 119 aa, Artificial Sequence
  Description of Artificial Sequence: Synthetic polypeptide
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 149 - protein, 108 aa
  Description of Artificial Sequence: Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

SEQ ID NO: 150 - protein, 449 aa, Artificial Sequence
  Description of Artificial Sequence: Synthetic polypeptide
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 214 aa, Artificial Sequence
  Description of Artificial Sequence: Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 448 aa, Artificial Sequence
  Description of Artificial Sequence: Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 214 aa, Artificial Sequence
  Description of Artificial Sequence: Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 470 aa, Artificial Sequence
  Description of Artificial Sequence: Synthetic polypeptide
MGWSCIILFLVATATGVHSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 238 aa, Artificial Sequence
  Description of Artificial Sequence: Synthetic polypeptide
MGWSCIILFLVATATGVHSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 445 aa, Artificial Sequence
  Description of Artificial Sequence: Synthetic polypeptide
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 217 aa, Artificial Sequence
  Description of Artificial Sequence: Synthetic polypeptide
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 158 - protein, 441 aa, Artificial Sequence
  Description of Artificial Sequence: Synthetic polypeptide
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 159 - protein, 215 aa, Artificial Sequence
  Description of Artificial Sequence: Synthetic polypeptide
ESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 160 - protein, 447 aa, Artificial Sequence
  Description of Artificial Sequence: Synthetic polypeptide
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 220 aa, Artificial Sequence
  Description of Artificial Sequence: Synthetic polypeptide
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 444 aa, Artificial Sequence
  Description of Artificial Sequence: Synthetic polypeptide
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 163 - protein, 214 aa, Artificial Sequence
  Description of Artificial Sequence: Synthetic polypeptide
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 449 aa, Artificial Sequence
  Description of Artificial Sequence: Synthetic polypeptide
EVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 165 - protein, 214 aa, Artificial Sequence
  Description of Artificial Sequence: Synthetic polypeptide
DIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 471 aa, Artificial Sequence
  Description of Artificial Sequence: Synthetic polypeptide
MEFGLSWVFLVALLRGVQCQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 236 aa, Artificial Sequence
  Description of Artificial Sequence: Synthetic polypeptide
MDMRVPAQLLGLLLLWFPGSRCDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 445 aa, Artificial Sequence
  Description of Artificial Sequence: Synthetic polypeptide
QVQLQESGPGLVKPSQTLSLTCSFSGFSLSTSGMGVGWIRQPSGKGLEWLAHIWWDGDESYNPSLKSRLTISKDTSKNQVSLKITSVTAADTAVYFCARNRYDPPWFVDWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 169 - protein, 214 aa, Artificial Sequence
  Description of Artificial Sequence: Synthetic polypeptide
DIQMTQSTSSLSASVGDRVTITCRASQDISNYLSWYQQKPGKAVKLLIYYTSKLHSGVPSRFSGSGSGTDYTLTISSLQQEDFATYFCLQGKMLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 119 aa, Artificial Sequence [sequence omitted]

SEQ ID NO: 171 - protein, 113 aa, Artificial Sequence [sequence omitted]

SEQ ID NO: 172 - protein, 449 aa, Artificial Sequence
  Description of Artificial Sequence: Synthetic polypeptide
QVTLRESGPALVKPTQTLTLTCTVSGFSLTSYSVHWVRQPPGKGLEWLGVSRIWASGGTDYNSALMLSISKDTSRNQVVLTMTNMDPVDTATYYCARDPPSSLLRLDYWGRGTLVTVSSASTKGPSVFPLAPSSKSTSGGTAALGCLVKDNSGALTYFPEPVTVSWSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQLPPSRDELPREPQVYTTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 449 aa, Artificial Sequence
  Description of Artificial Sequence: Synthetic polypeptide
QVTLRESGPALVKPTQTLTLTCTVSGFSLTSYSVHWVRQPPGKGLEWLGVIWASGGTDYNSALMSRLSISKDTSRNQVVLTMTNMDPVDTATYYCARDPPSSLLRLDYWGRGTPVTVSSASTKGPSVFPLAPSSKSTSGGTAALGCLVKDNSGALTYFPEPVTVSWSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPDSDGSENNYKTTPPVLFFLYSKLTVDKSRWQQGNVFSCSVMHEALHNHYTQKSLSLSPGK

SEQ ID NO: 174 - protein, 214 aa, Artificial Sequence
  Description of Artificial Sequence: Synthetic polypeptide
DIVMTQSPDSLAVSLGERATINCKSSQSLLNSGNQKNYLAWYQQKPGQPPKLLIYGASTRESGVPDRFSGSGSGTDFTLTISSLQAEDVAVYYCQNVHSFPFTFGGGTKLEIKRTVAAPSVFIFPPSDEQLKSGTASVVCLLNNFYPREAKVQWKVDNALQSGNSQESVTEQDSKDSTYSLSSTLTLSKEKHKVYACEVTHQSPVTKSFNRGEC

SEQ ID NO: 175 - protein, 5 aa, Artificial Sequence
  Description of Artificial Sequence: Synthetic peptide
SYSVH

SEQ ID NO: 176 - protein, 16 aa, Artificial Sequence
  Description of Artificial Sequence: Synthetic peptide
VIWASGGTDYNSALMS

SEQ ID NO: 177 - protein, 11 aa, Artificial Sequence
  Description of Artificial Sequence: Synthetic peptide
DPPSSLLRLDY

SEQ ID NO: 178 - protein, 17 aa, Artificial Sequence
  Description of Artificial Sequence: Synthetic peptide
KSSQSLLNSGNQKNYLA

SEQ ID NO: 179 - protein, 7 aa, Artificial Sequence
  Description of Artificial Sequence: Synthetic peptide
GASTRES

SEQ ID NO: 180 - protein, 9 aa, Artificial Sequence
  Description of Artificial Sequence: Synthetic peptide
QNVHSFPFT

SEQ ID NO: 181 - protein, 128 aa, Artificial Sequence
  Description of Artificial Sequence: Synthetic polypeptide
MSVPTQVLGLLLLWLTDARCDIQMTQSPSSLSASVGDRVTITCLASEGISSYLAWYQQKPGKAPKLLIYGANSLQTGVPSRFSGSGSATDYTLTISSLQPEDFATYYCQQSYKFPNTFGQGTKVEVKR

SEQ ID NO: 182 - protein, 135 aa, Artificial Sequence
  Description of Artificial Sequence: Synthetic polypeptide
MGWSCIILFLVATATGVHSEVQLVESGGGLVQPGGSLRLSCAVSGLSLTSNSVNWIRQAPGKGLEWVGLIWSNGDTDYNSAIKSRFTISRDTSKSTVYLQMNSLRAEDTAVYYCAREYYGYFDYWGQGTLVTVSS

SEQ ID NO: 183 - protein, 451 aa, Artificial Sequence
  Description of Artificial Sequence: Synthetic polypeptide
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 121 aa, Artificial Sequence [sequence omitted]

SEQ ID NO: 185 - protein, 214 aa, Artificial Sequence
  Description of Artificial Sequence: Synthetic polypeptide
DIQMTQSPSSLSASVGDRVTITCGTSEDIINYLNWYQQKPGKAPKLLIYHTSRLQSGVPSRFSGSGSGTDFTLTISSLQPEDFATYYCQQGYTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 186 - protein, 107 aa, Artificial Sequence [sequence omitted]

SEQ ID NO: 187 - protein, 449 aa, Artificial Sequence
  Description of Artificial Sequence: Synthetic polypeptide
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 213 aa, Artificial Sequence
  Description of Artificial Sequence: Synthetic polypeptide
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - protein, 106 aa, Artificial Sequence
  Description of Artificial Sequence: Synthetic polypeptide
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIK

SEQ ID NO: 190 - protein, 119 aa, Artificial Sequence
  Description of Artificial Sequence: Synthetic polypeptide
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 191 - protein, 10 aa, Artificial Sequence
  Description of Artificial Sequence: Synthetic peptide
SASISVSYMY

SEQ ID NO: 192 - protein, 7 aa, Artificial Sequence
  Description of Artificial Sequence: Synthetic peptide
DMSNLAS

SEQ ID NO: 193 - protein, 9 aa, Artificial Sequence
  Description of Artificial Sequence: Synthetic peptide
MQWSGYPYT

SEQ ID NO: 194 - protein, 10 aa, Artificial Sequence
  Description of Artificial Sequence: Synthetic peptide
GFTFSPFAMS

SEQ ID NO: 195 - protein, 17 aa, Artificial Sequence
  Description of Artificial Sequence: Synthetic peptide
KISPGGSWTYYSDTVTG

SEQ ID NO: 196 - protein, 10 aa, Artificial Sequence
  Description of Artificial Sequence: Synthetic peptide
QLWGYYALDI

SEQ ID NO: 197 - protein, 449 aa, Artificial Sequence
  Description of Artificial Sequence: Synthetic polypeptide
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 213 aa, Artificial Sequence
  Description of Artificial Sequence: Synthetic polypeptide
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 119 aa, Artificial Sequence [sequence omitted]

SEQ ID NO: 200 - protein, 106 aa, Artificial Sequence [sequence omitted]

SEQ ID NO: 201 - protein, 5 aa, Artificial Sequence
  Description of Artificial Sequence: Synthetic peptide
SFAMS

SEQ ID NO: 202 - protein, 17 aa, Artificial Sequence
  Description of Artificial Sequence: Synthetic peptide
EISSGGSYTYYPDTVTG

SEQ ID NO: 203 - protein, 10 aa, Artificial Sequence
  Description of Artificial Sequence: Synthetic peptide
GLWGYYALDY

SEQ ID NO: 204 - protein, 10 aa, Artificial Sequence
  Description of Artificial Sequence: Synthetic peptide
SASSSVSYMY

SEQ ID NO: 205 - protein, 7 aa, Artificial Sequence
  Description of Artificial Sequence: Synthetic peptide
DTSNLAS

SEQ ID NO: 206 - protein, 9 aa, Artificial Sequence
  Description of Artificial Sequence: Synthetic peptide
QQWSGYPYT

SEQ ID NO: 207 - protein, 446 aa, Artificial Sequence
  Description of Artificial Sequence: Synthetic polypeptide
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 214 aa, Artificial Sequence
  Description of Artificial Sequence: Synthetic polypeptide
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 116 aa, Artificial Sequence
  Description of Artificial Sequence: Synthetic polypeptide
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 210 - protein, 107 aa, Artificial Sequence
  Description of Artificial Sequence: Synthetic polypeptide
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 211 - protein, 5 aa, Artificial Sequence
  Description of Artificial Sequence: Synthetic peptide
AYSVN

SEQ ID NO: 212 - protein, 16 aa, Artificial Sequence
  Description of Artificial Sequence: Synthetic peptide
MIWGDGKIVYNSALKS

SEQ ID NO: 213 - protein, 10 aa, Artificial Sequence
  Description of Artificial Sequence: Synthetic peptide
DGYYPYAMDN

SEQ ID NO: 214 - protein, 15 aa, Artificial Sequence
  Description of Artificial Sequence: Synthetic peptide
RASKSVDSYGNSFMH

SEQ ID NO: 215 - protein, 7 aa, Artificial Sequence
  Description of Artificial Sequence: Synthetic peptide
LASNLES

SEQ ID NO: 216 - protein, 9 aa, Artificial Sequence
  Description of Artificial Sequence: Synthetic peptide
QQNNEDPRT

SEQ ID NO: 217 - protein, 117 aa, Artificial Sequence
  Description of Artificial Sequence: Synthetic polypeptide
VTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 218 - protein, 118 aa, Artificial Sequence [sequence omitted]

SEQ ID NO: 219 - protein, 112 aa, Artificial Sequence
  Description of Artificial Sequence: Synthetic polypeptide
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKR

SEQ ID NO: 220 - protein, 443 aa, Artificial Sequence
  Description of Artificial Sequence: Synthetic polypeptide
VTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 221 - protein, 444 aa, Artificial Sequence
  Description of Artificial Sequence: Synthetic polypeptide
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 222 - protein, 444 aa, Artificial Sequence
  Description of Artificial Sequence: Synthetic polypeptide
VTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 223 - protein, 445 aa, Artificial Sequence
  Description of Artificial Sequence: Synthetic polypeptide
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 224 - protein, 218 aa, Artificial Sequence
  Description of Artificial Sequence: Synthetic polypeptide
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 457 aa, Artificial Sequence
  Description of Artificial Sequence: Synthetic polypeptide
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 215 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 127 aa, Artificial Sequence [sequence omitted]

SEQ ID NO: 228 - protein, 109 aa, Artificial Sequence
  Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 229 - protein, 5 aa, Artificial Sequence
  Description of Artificial Sequence: Synthetic peptide
NYWMN

SEQ ID NO: 230 - protein, 16 aa, Artificial Sequence
  Description of Artificial Sequence: Synthetic peptide
AINQDGSEKYYVGSVK

SEQ ID NO: 231 - protein, 18 aa, Artificial Sequence
  Description of Artificial Sequence: Synthetic peptide
DYYDILTDYYIHYWYFDL

SEQ ID NO: 232 - protein, 12 aa, Artificial Sequence
  Description of Artificial Sequence: Synthetic peptide
RASQSVSSSYLA

SEQ ID NO: 233 - protein, 7 aa, Artificial Sequence
  Description of Artificial Sequence: Synthetic peptide
GASSRAT

SEQ ID NO: 234 - protein, 9 aa, Artificial Sequence
  Description of Artificial Sequence: Synthetic peptide
QQYGSSPCT

SEQ ID NO: 235 - protein, 445 aa, Artificial Sequence
  Description of Artificial Sequence: Synthetic polypeptide
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 236 - protein, 219 aa, Artificial Sequence
  Description of Artificial Sequence: Synthetic polypeptide
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 461 aa, Artificial Sequence
  Description of Artificial Sequence: Synthetic polypeptide
MEWTWRVLFLVAAATGAHSQVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 238 - protein, 234 aa, Artificial Sequence
  Description of Artificial Sequence: Synthetic polypeptide
MEAPAQLLFLLLLWLPDTTGEIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 5 aa, Artificial Sequence
  Description of Artificial Sequence: Synthetic peptide
RYGIS

SEQ ID NO: 240 - protein, 17 aa, Artificial Sequence
  Description of Artificial Sequence: Synthetic peptide
WISTYSGNTNYAQKLQG

SEQ ID NO: 241 - protein, 7 aa, Artificial Sequence
  Description of Artificial Sequence: Synthetic peptide
RQLYFDY

SEQ ID NO: 242 - protein, 11 aa, Artificial Sequence
  Description of Artificial Sequence: Synthetic peptide
RASQSVSSNLA

SEQ ID NO: 243 - protein, 7 aa, Artificial Sequence
  Description of Artificial Sequence: Synthetic peptide
DASTRAT

SEQ ID NO: 244 - protein, 9 aa, Artificial Sequence
  Description of Artificial Sequence: Synthetic peptide
QQYDNWPLT

SEQ ID NO: 245 - protein, 446 aa, Artificial Sequence
  Description of Artificial Sequence: Synthetic polypeptide
QVQLVQSGAEVKKPGASVKVSCKASGYIFITYWMTWVRQAPGQGLEWMGQIFPASGSADYNEKFEGRVTMTTDTSTSTAYMELRSLRSDDTAVYYCARGGGGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - protein, 214 aa, Artificial Sequence
  Description of Artificial Sequence: Synthetic polypeptide
DIQMTQSPSSLSASVGDRVTITCRTSENIYSYLAWYQQKPGKAPKLLIYNAKTLAEGVPSRFSGSGSGTDFTLTISSLQPEDFATYYCQHHYGIPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 11 aa, Artificial Sequence
  Description of Artificial Sequence: Synthetic peptide
RTSENIYSYLA

SEQ ID NO: 248 - protein, 7 aa, Artificial Sequence
  Description of Artificial Sequence: Synthetic peptide
NAKTLAE

SEQ ID NO: 249 - protein, 9 aa, Artificial Sequence
  Description of Artificial Sequence: Synthetic peptide
QHHYGIPFT

SEQ ID NO: 250 - protein, 10 aa, Artificial Sequence
  Description of Artificial Sequence: Synthetic peptide
GYIFITYWMT

SEQ ID NO: 251 - protein, 16 aa, Artificial Sequence
  Description of Artificial Sequence: Synthetic peptide
QIFPASGSADYNEMFE

SEQ ID NO: 252 - protein, 16 aa, Artificial Sequence
  Description of Artificial Sequence: Synthetic peptide
QIFPASGSADYNEKFE

SEQ ID NO: 253 - protein, 16 aa, Artificial Sequence
  Description of Artificial Sequence: Synthetic peptide
QIFPASGSADYAQKLQ

SEQ ID NO: 254 - protein, 7 aa, Artificial Sequence
  Description of Artificial Sequence: Synthetic peptide
GGGGFAY

SEQ ID NO: 255 - protein, 450 aa, Artificial Sequence
  Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGYYDILTGYYYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 256 - protein, 214 aa, Artificial Sequence
  Description of Artificial Sequence: Synthetic polypeptide
EIVLTQSPATLSLSPGERATLSCRASQSVSRYLAWYQQKPGQAPRLLIYDASNRATGIPARFSGSGSGTDSTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 257 - protein, 449 aa, Artificial Sequence
  Description of Artificial Sequence: Synthetic polypeptide
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 214 aa, Artificial Sequence
  Description of Artificial Sequence: Synthetic polypeptide
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - protein, 119 aa, Artificial Sequence
  Description of Artificial Sequence: Synthetic polypeptide
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSS

SEQ ID NO: 260 - protein, 107 aa, Artificial Sequence
  Description of Artificial Sequence: Synthetic polypeptide
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIK

SEQ ID NO: 261 - protein, 118 aa, Artificial Sequence
  Description of Artificial Sequence: Synthetic polypeptide
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVS

SEQ ID NO: 262 - protein, 109 aa, Artificial Sequence
  Description of Artificial Sequence: Synthetic polypeptide
DIQMTQSPSSLSASVGDRVTITCKASRDIRSYLTWYQQKPGKAPKTLIYYATSLADGVPSRFSGSGSGQDYSLTISSLESDDTATYYCLQHGESPFTFGSGTKLEIKRA

SEQ ID NO: 263 - protein, 107 aa, Artificial Sequence
  Description of Artificial Sequence: Synthetic polypeptide
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 264 - protein, 122 aa, Artificial Sequence [sequence omitted]

SEQ ID NO: 265 - protein, 213 aa, Artificial Sequence
  Description of Artificial Sequence: Synthetic polypeptide
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 452 aa, Artificial Sequence
  Description of Artificial Sequence: Synthetic polypeptide
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 219 aa, Artificial Sequence
  Description of Artificial Sequence: Synthetic polypeptide
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKVQIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 446 aa, Artificial Sequence
  Description of Artificial Sequence: Synthetic polypeptide
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 269 - protein, 456 aa, Artificial Sequence
  Description of Artificial Sequence: Synthetic polypeptide
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 270 - protein, 215 aa, Artificial Sequence
  Description of Artificial Sequence: Synthetic polypeptide
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 442 aa, Artificial Sequence
  Description of Artificial Sequence: Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 272 - protein, 219 aa, Artificial Sequence
  Description of Artificial Sequence: Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 696 aa, Artificial Sequence
  Description of Artificial Sequence: Synthetic polypeptide
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTEFAGAAAVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 274 - protein, 1684 aa, Artificial Sequence
  Description of Artificial Sequence: Synthetic polypeptide
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 275 - protein, 672 aa, Artificial Sequence
  Description of Artificial Sequence: Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 276 - protein, 233 aa, Artificial Sequence
  Description of Artificial Sequence: Synthetic polypeptide
SEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 277 - protein, 446 aa, Artificial Sequence
  Description of Artificial Sequence: Synthetic polypeptide
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 278 - protein, 214 aa, Artificial Sequence
  Description of Artificial Sequence: Synthetic polypeptide
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 116 aa, Artificial Sequence [sequence omitted]

SEQ ID NO: 280 - protein, 107 aa, Artificial Sequence [sequence omitted]

SEQ ID NO: 281 - protein, 449 aa, Artificial Sequence
  Description of Artificial Sequence: Synthetic polypeptide
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 217 aa, Artificial Sequence
  Description of Artificial Sequence: Synthetic polypeptide
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 119 aa, Artificial Sequence
  Description of Artificial Sequence: Synthetic polypeptide
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 284 - protein, 110 aa, Artificial Sequence
  Description of Artificial Sequence: Synthetic polypeptide
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 285 - protein, 449 aa, Artificial Sequence
  Description of Artificial Sequence: Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 286 - protein, 219 aa, Artificial Sequence
  Description of Artificial Sequence: Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 287 - protein, 119 aa, Artificial Sequence
  Description of Artificial Sequence: Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 288 - protein, 112 aa, Artificial Sequence
  Description of Artificial Sequence: Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 289 - protein, 449 aa, Artificial Sequence
  Description of Artificial Sequence: Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 290 - protein, 214 aa, Artificial Sequence
  Description of Artificial Sequence: Synthetic polypeptide
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 291 - protein, 119 aa, Artificial Sequence
  Description of Artificial Sequence: Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 292 - protein, 107 aa, Artificial Sequence
  Description of Artificial Sequence: Synthetic polypeptide
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 293 - protein, 5 aa, Artificial Sequence
  Description of Artificial Sequence: Synthetic peptide
RYWMS

SEQ ID NO: 294 - protein, 17 aa, Artificial Sequence
  Description of Artificial Sequence: Synthetic peptide
EINPDSSTINYAPSLKD

SEQ ID NO: 295 - protein, 10 aa, Artificial Sequence
  Description of Artificial Sequence: Synthetic peptide
PDGNYWYFDV

SEQ ID NO: 296 - protein, 11 aa, Artificial Sequence
  Description of Artificial Sequence: Synthetic peptide
KASQDVGIAVA

SEQ ID NO: 297 - protein, 7 aa, Artificial Sequence
  Description of Artificial Sequence: Synthetic peptide
WASTRHT

SEQ ID NO: 298 - protein, 9 aa, Artificial Sequence
  Description of Artificial Sequence: Synthetic peptide
QQYSSYPYT

SEQ ID NO: 299 - protein, 467 aa, Artificial Sequence
  Description of Artificial Sequence: Synthetic polypeptide
MDFGFSLVFLALILKGVQCEVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 300 - protein, 121 aa, Artificial Sequence
  Description of Artificial Sequence: Synthetic polypeptide
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 301 - protein, 5 aa, Artificial Sequence
  Description of Artificial Sequence: Synthetic peptide
NYWIH

SEQ ID NO: 302 - protein, 17 aa, Artificial Sequence
  Description of Artificial Sequence: Synthetic peptide
GINPGNNYATYRRKFQG

SEQ ID NO: 303 - protein, 12 aa, Artificial Sequence
  Description of Artificial Sequence: Synthetic peptide
EGYGNYGAWFAY

SEQ ID NO: 304 - protein, 239 aa, Artificial Sequence
  Description of Artificial Sequence: Synthetic polypeptide
MKLPVRLLVLLLFWIPASRGDVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 305 - protein, 113 aa, Artificial Sequence
  Description of Artificial Sequence: Synthetic polypeptide
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 306 - protein, 16 aa, Artificial Sequence
  Description of Artificial Sequence: Synthetic peptide
RSSQSLANSYGNTFLS

SEQ ID NO: 307 - protein, 7 aa, Artificial Sequence
  Description of Artificial Sequence: Synthetic peptide
GISNRFS

SEQ ID NO: 308 - protein, 9 aa, Artificial Sequence
  Description of Artificial Sequence: Synthetic peptide
LQGTHQPYT

SEQ ID NO: 309 - protein, 123 aa, Artificial Sequence
  Description of Artificial Sequence: Synthetic polypeptide
EVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGTHWGVLFAYWGQGTLVTVSA

SEQ ID NO: 310 - protein, 476 aa, Artificial Sequence
  Description of Artificial Sequence: Synthetic polypeptide
MEVQLVESGGGLVKPGGSLKLSCAASGFAFSIYDMSWVRQTPEKCLEWVAYISSGGGTTYYPDTVKGRFTISRDNAKNTLYLQMSSLKSEDTAMYYCARHSGYGTHWGVLFAYWGQGTLVTVSAKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 311 - protein, 8 aa, Artificial Sequence
  Description of Artificial Sequence: Synthetic peptide
GFAFSIYD

SEQ ID NO: 312 - protein, 8 aa, Artificial Sequence
  Description of Artificial Sequence: Synthetic peptide
ISSGGGTT

SEQ ID NO: 313 - protein, 16 aa, Artificial Sequence
  Description of Artificial Sequence: Synthetic peptide
ARHSGYGTHWGVLFAY

SEQ ID NO: 314 - protein, 107 aa, Artificial Sequence
  Description of Artificial Sequence: Synthetic polypeptide
DIQMTQTTSSLSASLGDRVTISCRASQDIHGYLNWYQQKPDGTVKLLIYYTSILHSGVPSRFSGSGSGTDYSLTISNLEQEDFATYFCQQGNTLPWTFGGGTKLEIK

SEQ ID NO: 315 - protein, 107 aa, Artificial Sequence
  Description of Artificial Sequence: Synthetic polypeptide
DIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIK

SEQ ID NO: 316 - protein, 108 aa, Artificial Sequence
  Description of Artificial Sequence: Synthetic polypeptide
MDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGCGTKLEIK

SEQ ID NO: 317 - protein, 6 aa, Artificial Sequence
  Description of Artificial Sequence: Synthetic peptide
QDIHGY

SEQ ID NO: 318 - protein, 9 aa, Artificial Sequence
  Description of Artificial Sequence: Synthetic peptide
QQGNTLPWT

SEQ ID NO: 319 - protein, 121 aa, Artificial Sequence [sequence omitted]

SEQ ID NO: 320 - protein, 451 aa, Artificial Sequence
  Description of Artificial Sequence: Synthetic polypeptide
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 321 - protein, 7 aa, Artificial Sequence
  Description of Artificial Sequence: Synthetic peptide
SGDYYWS

SEQ ID NO: 322 - protein, 16 aa, Artificial Sequence
  Description of Artificial Sequence: Synthetic peptide
YIYYSGSTDYNPSLKS

SEQ ID NO: 323 - protein, 11 aa, Artificial Sequence
  Description of Artificial Sequence: Synthetic peptide
VSIFGVGTFDY

SEQ ID NO: 324 - protein, 214 aa, Artificial Sequence
  Description of Artificial Sequence: Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 325 - protein, 107 aa, Artificial Sequence [sequence omitted]

SEQ ID NO: 326 - protein, 11 aa, Artificial Sequence
  Description of Artificial Sequence: Synthetic peptide
RASQSVSSYLA

SEQ ID NO: 327 - protein, 7 aa, Artificial Sequence
  Description of Artificial Sequence: Synthetic peptide
DASNRAT

SEQ ID NO: 328 - protein, 9 aa, Artificial Sequence
  Description of Artificial Sequence: Synthetic peptide
HQYGSTPLT

SEQ ID NO: 329 - protein, 446 aa, Artificial Sequence
  Description of Artificial Sequence: Synthetic polypeptide
QVQLQESGPGLVKPSETLSLTCTVSGGSISIYYWSWIRQPPGKGLEWIGYVYYSGSTNYNPSLKSRVTISVDTSKNQFSLKLNSVTAADTAVYYCARGGYDFWSGYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 330 - protein, 5 aa, Artificial Sequence
  Description of Artificial Sequence: Synthetic peptide
IYYWS

SEQ ID NO: 331 - protein, 16 aa, Artificial Sequence
  Description of Artificial Sequence: Synthetic peptide
YVYYSGSTNYNPSLKS

SEQ ID NO: 332 - protein, 12 aa, Artificial Sequence
  Description of Artificial Sequence: Synthetic peptide
GGYDFWSGYFDY

SEQ ID NO: 333 - protein, 215 aa, Artificial Sequence
  Description of Artificial Sequence: Synthetic polypeptide
EIVMTQSPATLSVSPGERATLSCRASQSVDSNLAWYRQKPGQAPRLLIYGASTRATGIPARFSGSGSGTEFTLTISSLQSEDFAVYYCQQYINWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 11 aa, Artificial Sequence
  Description of Artificial Sequence: Synthetic peptide
RASQSVDSNLA

SEQ ID NO: 335 - protein, 7 aa, Artificial Sequence
  Description of Artificial Sequence: Synthetic peptide
GASTRAT

SEQ ID NO: 336 - protein, 10 aa, Artificial Sequence
  Description of Artificial Sequence: Synthetic peptide
QQYINWPPIT

SEQ ID NO: 337 - protein, 119 aa, Artificial Sequence [sequence omitted]

SEQ ID NO: 338 - protein, 449 aa, Artificial Sequence
  Description of Artificial Sequence: Synthetic polypeptide
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 339 - protein, 10 aa, Artificial Sequence
  Description of Artificial Sequence: Synthetic peptide
GYTFTSYWLH

SEQ ID NO: 340 - protein, 18 aa, Artificial Sequence
  Description of Artificial Sequence: Synthetic peptide
GMIDPSNSDTRFNPNFKD

SEQ ID NO: 341 - protein, 12 aa, Artificial Sequence
  Description of Artificial Sequence: Synthetic peptide
ATYRSYVTPLDY

SEQ ID NO: 342 - protein, 114 aa, Artificial Sequence
  Description of Artificial Sequence: Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 343 - protein, 220 aa, Artificial Sequence
  Description of Artificial Sequence: Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 344 - protein, 17 aa, Artificial Sequence
  Description of Artificial Sequence: Synthetic peptide
KSSQSLLYTSSQKNYLA

SEQ ID NO: 345 - protein, 7 aa, Artificial Sequence
  Description of Artificial Sequence: Synthetic peptide
WASTRES

SEQ ID NO: 346 - protein, 9 aa, Artificial Sequence
  Description of Artificial Sequence: Synthetic peptide
QQYYAYPWT

SEQ ID NO: 347 - protein, 447 aa, Artificial Sequence
  Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 348 - protein, 218 aa, Artificial Sequence
  Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 5 aa, Artificial Sequence
  Description of Artificial Sequence: Synthetic peptide
NYYMY

SEQ ID NO: 350 - protein, 17 aa, Artificial Sequence
  Description of Artificial Sequence: Synthetic peptide
GINPSNGGTNFNEKFKN

SEQ ID NO: 351 - protein, 11 aa, Artificial Sequence
  Description of Artificial Sequence: Synthetic peptide
RDYRFDMGFDY

SEQ ID NO: 352 - protein, 15 aa, Artificial Sequence
  Description of Artificial Sequence: Synthetic peptide
RASKGVSTSGYSYLH

SEQ ID NO: 353 - protein, 7 aa, Artificial Sequence
  Description of Artificial Sequence: Synthetic peptide
LASYLES

SEQ ID NO: 354 - protein, 9 aa, Artificial Sequence
  Description of Artificial Sequence: Synthetic peptide
QHSRDLPLT

SEQ ID NO: 355 - protein, 113 aa, Artificial Sequence
  Description of Artificial Sequence: Synthetic polypeptide
EVQLVQSGAEVEKPGASVKISCKASGSSFTGYNMNWVRQNIGKSLEWIGAIDPYYGGTSYNQKFKGRATLTVDKSTSTAYMHLKSLRSEDTAVYYCVSGMEYWGQGTSVTVSS

SEQ ID NO: 356 - protein, 113 aa, Artificial Sequence
  Description of Artificial Sequence: Synthetic polypeptide
DVVMTQTPLSLPVTPGEPASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 357 - protein, 214 aa, Artificial Sequence
  Description of Artificial Sequence: Synthetic polypeptide
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 358 - protein, 445 aa, Artificial Sequence
  Description of Artificial Sequence: Synthetic polypeptide
QVQLQQSGAELVKPGASVKLSCKASGYTFTSYDINWVRQRPEQGLEWIGWIFPGDGSTKYNEKFKGKATLTTDKSSSTAYMQLSRLTSEDSAVYFCAREDYYDNSYYFDYWGQGTTLTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 359 - protein, 451 aa, Artificial Sequence
  Description of Artificial Sequence: Synthetic polypeptide
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 360 - protein, 124 aa, Artificial Sequence
  Description of Artificial Sequence: Synthetic polypeptide
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 361 - protein, 8 aa, Artificial Sequence
  Description of Artificial Sequence: Synthetic peptide
GFTFRDYA

SEQ ID NO: 362 - protein, 8 aa, Artificial Sequence
  Description of Artificial Sequence: Synthetic peptide
ISGSGGNT

SEQ ID NO: 363 - protein, 18 aa, Artificial Sequence
  Description of Artificial Sequence: Synthetic peptide
AKDRLSITIRPRYYGLDV

SEQ ID NO: 364 - protein, 219 aa, Artificial Sequence
  Description of Artificial Sequence: Synthetic polypeptide
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 365 - protein, 112 aa, Artificial Sequence [sequence omitted]

SEQ ID NO: 366 - protein, 11 aa, Artificial Sequence
  Description of Artificial Sequence: Synthetic peptide
QSLLYSIGYNY

SEQ ID NO: 367 - protein, 9 aa, Artificial Sequence
  Description of Artificial Sequence: Synthetic peptide
MQALQTPYT

SEQ ID NO: 368 - protein, 215 aa, Artificial Sequence
  Description of Artificial Sequence: Synthetic polypeptide
DIQLTQSPSSLSASVGDRVTMTCSASSSVSSSYLYWYQQKPGKAPKLWIYSTSNLASGVPARFSGSGSGTDFTLTISSLQPEDSASYFCHQWNRYPYTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 369 - protein, 449 aa, Artificial Sequence
  Description of Artificial Sequence: Synthetic polypeptide
QVQLQQSGAEVKKPGASVKVSCEASGYTFPSYVLHWVKQAPGQGLEWIGYINPYNDGTQYNEKFKGKATLTRDTSINTAYMELSRLRSDDTAVYYCARGFGGSYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 12 aa, Artificial Sequence
  Description of Artificial Sequence: Synthetic peptide
SASSSVSSSYLY

SEQ ID NO: 371 - protein, 7 aa, Artificial Sequence
  Description of Artificial Sequence: Synthetic peptide
STSNLAS

SEQ ID NO: 372 - protein, 9 aa, Artificial Sequence
  Description of Artificial Sequence: Synthetic peptide
HQWNRYPYT

SEQ ID NO: 373 - protein, 5 aa, Artificial Sequence
  Description of Artificial Sequence: Synthetic peptide
SYVLH

SEQ ID NO: 374 - protein, 17 aa, Artificial Sequence
  Description of Artificial Sequence: Synthetic peptide
YINPYNDGTQYNEKFKG

SEQ ID NO: 375 - protein, 10 aa, Artificial Sequence
  Description of Artificial Sequence: Synthetic peptide
GFGGSYGFAY

SEQ ID NO: 376 - DNA, 60 bp, Artificial Sequence
  Description of Artificial Sequence: Synthetic oligonucleotide
gaagctgctgcaagagaagctgcagctagggaggctgcagctagggaggctgctgcaaga

SEQ ID NO: 377 - DNA, 18 bp, Artificial Sequence
  Description of Artificial Sequence: Synthetic oligonucleotide
ggtagcggcagcggtagc

SEQ ID NO: 378 - DNA, 30 bp, Artificial Sequence
  Description of Artificial Sequence: Synthetic oligonucleotide
ggtgaaaatttgtattttcaatctggtggt

SEQ ID NO: 379 - DNA, 24 bp, Artificial Sequence
  Description of Artificial Sequence: Synthetic oligonucleotide
tccgcttgttactgtgagctttcc

SEQ ID NO: 380 - DNA, 45 bp, Artificial Sequence
  Description of Artificial Sequence: Synthetic oligonucleotide
ggtggaggaggttctggaggcggtggaagtggtggcggaggtagc

SEQ ID NO: 381 - DNA, 12 bp, Artificial Sequence
  Description of Artificial Sequence: Synthetic oligonucleotide
ggtggttctggt

SEQ ID NO: 382 - DNA, 24 bp, Artificial Sequence
  Description of Artificial Sequence: Synthetic oligonucleotide
ggtggttctggtggtggttctggt

SEQ ID NO: 383 - DNA, 36 bp, Artificial Sequence
  Description of Artificial Sequence: Synthetic oligonucleotide
ggtggttctggtggtggttctggtggtggttctggt

SEQ ID NO: 384 - DNA, 108 bp, Artificial Sequence
  Description of Artificial Sequence: Synthetic polynucleotide
ggtggttctgccggtggctccggttctggctccagcggtggcagctctggtgcgtccggcacgggtactgcgggtggcactggcagcggttccggtactggctctggc

SEQ ID NO: 385 - DNA, 108 bp, Artificial Sequence
  Description of Artificial Sequence: Synthetic polynucleotide
ggtggttctggcggcggttctgaaggtggcggctccgaaggcggcggcagcgagggcggtggtagcgaaggtggtggctccgagggtggcggttccggcggcggtagc

SEQ ID NO: 386 - protein, 22 aa, Artificial Sequence
  Description of Artificial Sequence: Synthetic peptide
GSGATNFSLLKQAGDVEENPGP

SEQ ID NO: 387 - DNA, 66 bp, Artificial Sequence
  Description of Artificial Sequence: Synthetic oligonucleotide
ggaagcggagctactaacttcagcctgctgaagcaggctggagacgtggaggagaaccctggacct

SEQ ID NO: 388 - protein, 5 aa, Artificial Sequence
  Description of Artificial Sequence: Synthetic peptide
EAAAR

SEQ ID NO: 389 - protein, 6 aa, Artificial Sequence
  Description of Artificial Sequence: Synthetic peptide
GSGSGS

SEQ ID NO: 390 - protein, 4 aa, Artificial Sequence
  Description of Artificial Sequence: Synthetic peptide
GGSG

SEQ ID NO: 391 - protein, 8 aa, Artificial Sequence
  Description of Artificial Sequence: Synthetic peptide
GGSGGGSG

SEQ ID NO: 392 - protein, 12 aa, Artificial Sequence
  Description of Artificial Sequence: Synthetic peptide
GGSGGGSGGGSG

SEQ ID NO: 393 - DNA, 250 bp, Artificial Sequence
  Description of Artificial Sequence: Synthetic polynucleotide
  misc_feature (1)..(250): This sequence may be 80-250 nucleotides in length
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 394 - DNA, 30 bp, Artificial Sequence
  Description of Artificial Sequence: Synthetic oligonucleotide
  See specification as filed for detailed description of substitutions and preferred embodiments
aaaaaaaaaaaaaaaaaaaaaaaaaaaaaa

SEQ ID NO: 395 - DNA, 120 bp, Artificial Sequence
  Description of Artificial Sequence: Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa